(19) United States
(12) Patent Application Publication
(10) Pub. No.: US 2023/0382889 A1
(43) Pub. Date: Nov. 30, 2023

(54) COMPOUNDS FOR INHIBITING KIF18A

(71) Applicant: Volastra Therapeutics, Inc., New York, NY (US)

(72) Inventor: Derek A. COGAN, New York, NY (US)

(73) Assignee: Volastra Therapeutics, Inc., New York, NY (US)

(21) Appl. No.: 18/140,533

(22) Filed: Apr. 27, 2023

Related U.S. Application Data
(60) Provisional application No. 63/456,342, filed on Mar. 31, 2023.
(60) Provisional application No. 63/389,237, filed on Jul. 14, 2022.
(60) Provisional application No. 63/336,183, filed on Apr. 28, 2022.

(51) Int. Cl. C07D 401/14; C07D 405/14; C07D 417/14; C07D 491/08; C07D 491/107; C07D 401/10; C07D 413/14; A61P 35/00

(52) U.S. Cl. CPC C07D 401/14; C07D 405/14; C07D 417/14; C07D 491/08; C07D 491/107; C07D 401/10; C07D 413/14; A61P 35/00

(57) ABSTRACT

The present disclosure relates generally to inhibitors of KIF18A, compositions thereof, and methods of using said compounds and compositions thereof. More specifically, the present disclosure relates to inhibitors of KIF18A and methods of their use for treating disease mediated by KIF18A, such as cancer.

### CROSS-REFERENCE TO RELATED APPLICATIONS

This application claims priority to and benefit of U.S. Provisional Patent Application No. 63/336,183, filed Apr. 28, 2022; U.S. Provisional Patent Application No. 63/389,237, filed Jul. 14, 2022; and U.S. Provisional Patent Application No. 63/456,342, filed Mar. 31, 2023, the disclosures of each of which are hereby incorporated herein by reference in their entirety.

### FIELD

The present disclosure relates generally to inhibitors of KIF18A, compositions thereof, and methods of using said compounds and compositions thereof. More specifically, the present disclosure relates to inhibitors of KIF18A and methods of their use for treating disease mediated by KIF18A, such as cancer.

### BACKGROUND

KIF18A is a kinesin involved in assisting the kinetochore-microtubule (kt-MT) attachment and chromosomal alignment during cell mitosis. Its cargo domain binds directly to protein phosphatase 1 (PP1) and carries it to the plus end of MT where PP1 dephosphorylates Hec1, a kinetochore complex component, further enhancing kt-MT attachment throughout metaphase and anaphase. Its MT-binding motor domain has ATPase activity that powers the KIF18A translocation along MT lattice, enhanced by its C-terminal MT-binding site, and caps and depolymerizes growing microtubule at the plus end, thus dampening MT dynamics. This modulation of MT dynamics by KIF18A often occurs at the following (or trailing) sister chromatid, thereby providing a counterbalancing tension to the leading sister chromatid movement catalyzed by another kinesin Kif2C/MCAK. Loss of KIF18A function causes defective kt-MT attachments and loss of tension within the spindle in cells of high chromosome instability (CIN), leading to hyper stable, longer and multipolar spindles, mitotic arrest, centrosome fragmentation and spindle assembly checkpoint activation or cell death. KIF18A is identified from DEPMAP RNAi data re-analysis as one of the top candidates essential for CIN-high cells. Reported synthetic lethality screens also singled out KIF18A as a potential anticancer target whose knockdown preferentially renders CIN-high (but not CIN-low), aneuploid and whole-genome doubled cells vulnerable to death. Cellular toxicity assay in isogenic cell lines confirmed the enhanced sensitivity of CIN-high cells to KIF18A inhibitors. Ongoing in vivo mouse models using KIF18A inhibitor or knockdown demonstrated effect of inhibited tumor growth. Thus, there is a need for new compounds for use in treating diseases mediated by KIF18A.

### BRIEF SUMMARY

The present disclosure provides compounds of Formula (I) and Formula (II), Formula (III), compositions thereof, and methods of using said compounds and compositions thereof for the treatment of diseases or conditions associated with KIF18a. In one aspect, provided are compounds of Formula (I):

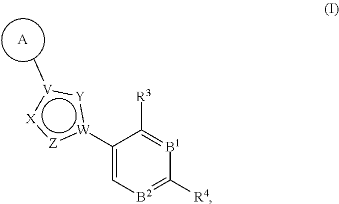

or a pharmaceutically acceptable salt thereof, wherein: X and Z are independently O, N, or CH; Y is NH, N, or CH; V and W are independently N or C; wherein at least one of X and Z is N or Y is NH; Ring A is

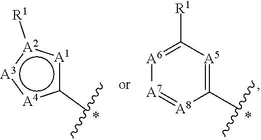

wherein one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl; A² is N or C; A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O; wherein “*” indicates the point of attachment to V; B¹ and B² are each independently N, CH or CR^{B}, wherein R^{B} is halogen; R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20}, wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl; R^{a1}-R^{a10} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or R^{a14} and R^{a15} are taken together with the nitrogen to which they are attached to form a 3- to 10-membered heterocycloalkyl optionally substituted with one or more halo; each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or R¹ and the R² of A⁵ are taken together with the carbon atoms to which they are attached to form a C₃-C₆ cycloalkyl or a 3- to 10-membered heterocycloalkyl; R³ is piperidinyl, pyrrolidinyl, or azepanyl, wherein the piperidinyl, the pyrrolidinyl, or the azepanyl, is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or azepanyl, or wherein the piperidinyl, pyrrolidinyl, or azepanyl are optionally substituted with a C₁₋₂ alkylene to form a bridged piperidinyl, pyrrolidinyl, or azepanyl ring system, wherein the piperidinyl, the pyrrolidinyl, the azepanyl, or the spirocyclic, fused, or bridged bicyclic ring system formed by the C₃₋₁₀ cycloalkyl, 3- to 10-membered heterocycloalkyl, or C₁₋₂ alkylene with piperidinyl, pyrrolidinyl, or azepanyl is optionally substituted with one or more substituents independently selected from the group consisting of C₁-C₃ alkyl, C₁-C₃ haloalkyl, and halo; R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH. In some embodiments, when X is N, Y is N, and Z is O, then R⁴ is not H.

In another aspect, provided is a compound of Formula (II).

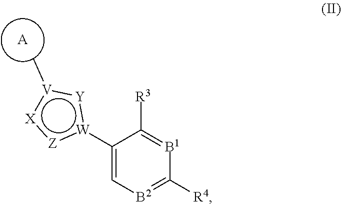

or a pharmaceutically acceptable salt thereof, wherein: X and Z are independently O, N, or CH; Y is NH or CH; V and W are independently N or C; wherein at least one of X and Z is N or Y is NH; Ring A is

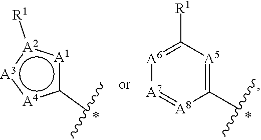

wherein one or two of A¹, A³, and A⁴ are independently N, O, or S, and the remaining one or two of A¹, A³, and A⁴ are independently CH or CR²; A² is N or C; A⁵-A⁸ are independently CH, CR² or N, wherein at least two of A⁵, A⁶, A⁷, and A⁸ are CH or CR², and the remaining one or two of A⁵, A⁶, A⁷, and A⁸, if present, are N; wherein “*” indicates the point of attachment to V; B¹ and B² are each independently N or CH, R¹ is C₁₋₆ alkyl, C₃₋₆ cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20}, wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R¹ is optionally substituted with one or more halogens; R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or R^{a14} and R^{a15} are taken together with the nitrogen to which they are attached to form a 3- to 10-membered heterocycloalkyl optionally substituted with one or more halo; each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein R^{b1} and R^{b2} are independently optionally substituted with C₁-C₃ alkyl or R^{b1} and R^{b2} are taken together with the nitrogen to which they are attached to form a 3-to 6-membered ring; or R¹ and the R² of A⁵ are taken together with the carbon atoms to which they are attached to form a C₃-C₆ cycloalkyl or a 3- to 6-membered heterocycloalkyl; R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or the azepanyl, and wherein the piperidinyl, the pyrrolidinyl, the azepanyl or the spirocyclic or fused bicyclic ring system formed by the C₃₋₁₀ cycloalkyl or 3- to 10-membered heterocycloalkyl with piperidinyl, pyrrolidinyl, or azepanyl is optionally substituted with one or more substituents independently selected from the group consisting of C₁-C₃ alkyl and C₁-C₃ haloalkyl; R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

In yet another aspect, provided herein is a compound of Formula (III)

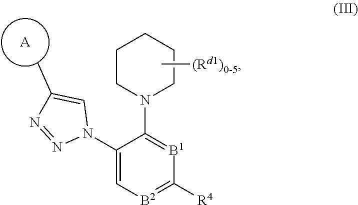

or a pharmaceutically acceptable salt thereof, wherein: Ring A is

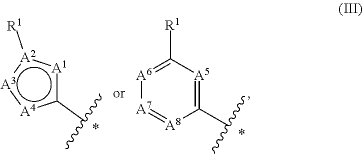

wherein one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl; A² is N or C; A⁵-A⁸ are independently CH, CR², N, or NR², wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O; wherein “*” indicates the point of attachment to V; B¹ and B² are each independently N, CH or CR⁸, wherein R^{B} is halogen; R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or (CR^{a17}R^{a18})₀₋₁C(O)NR^{a19}R^{a20}, wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl; R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or R^{a14} and R^{a15} are taken together with the nitrogen to which they are attached to form a 3- to 10-membered heterocycloalkyl optionally substituted with one or more halo; each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or R¹ and the R² of A⁵ are taken together with the carbon atoms to which they are attached to form a C₃-C₆ cycloalkyl or a 3- to 10-membered heterocycloalkyl; wherein each R^{d1} is independently selected from the group consisting of C₁-C₃ alkyl, C₁-C₃ haloalkyl, and halo; or wherein two R^{d1} are taken together to form a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl; or wherein two R^{d1} are taken together to form a C₁₋₂ alkylene, wherein the C₁₋₂ alkylene forms a bridged piperidinyl ring system, wherein the spirocyclic, fused, or bridged bicyclic ring system formed by the C₃₋₁₀ cycloalkyl, 3- to 10-membered heterocycloalkyl, or C₁₋₂ alkylene with the piperidinyl is optionally substituted with one or more substituents independently selected from the group consisting of C₁-C₃ alkyl, C₁-C₃ haloalkyl, and halo; R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH.

In another aspect, provided is pharmaceutical composition comprising a compound of Formula (I), a compound of Formula (II), a compound of Formula (III), or a compound of Table 1, or a pharmaceutically acceptable salt thereof, and a pharmaceutically acceptable carrier or excipient.

In another aspect, provided herein is a method of inhibiting KIF18A comprising contacting a cell with an effective amount of a compound or a pharmaceutical composition as described herein.

In another aspect, provided herein are methods of treating or preventing a disease or condition in an individual, comprising administering to the subject a therapeutically effective amount of a compound or a pharmaceutical composition as described herein. In some embodiments, the disease or condition is mediated by KIF18A. In some embodiments, the disease or condition is cancer. In some embodiments, the disease or condition is a cellular proliferation disorder.

### DESCRIPTION OF THE FIGURES

The drawings illustrate certain features and advantages of this disclosure. These embodiments are not intended to limit the scope of the appended claims in any manner.

FIG. 1 depicts a plot of tumor volume over time in an in vivo xenograft ovarian cancer cell line model (OVCAR3) in Balb/c mice treated with Compound 47 (at doses of 3, 10 and 30 mg/kg QD PO) as compared to vehicle control.

FIG. 2 depicts a plot of tumor volume over time in an in vivo xenograft non-small cell lung carcinoma cell line model (HCC15) in Balb/c mice treated with Compound 47 (at doses of 3, 10 and 30 mg/kg QD PO) as compared to vehicle control.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

As used in the present specification, the following words and phrases are generally intended to have the meanings as set forth below, except to the extent that the context in which they are used indicates otherwise.

Throughout this application, unless the context indicates otherwise, references to a compound of Formula (I) includes all subgroups of Formula (I) defined herein, including all substructures, subgenera, preferences, embodiments, examples and particular compounds defined and/or described herein. In some embodiments, references to a compound of Formula (I) and subgroups thereof include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a compound of Formula (I) and subgroups thereof, include polymorphs, solvates, co-crystals, isomers, tautomers and/or oxides thereof. In some embodiments, references to a compound of Formula (I) and subgroups thereof include polymorphs, solvates, and/or co-crystals thereof. In some embodiments, references to a compound of Formula (I) and subgroups thereof, include isomers, tautomers and/or oxides thereof. In some embodiments, references to a compound of Formula (I) and subgroups thereof include solvates thereof.

“Alkyl” encompasses straight and branched carbon chains having the indicated number of carbon atoms, for example, from 1 to 20 carbon atoms, or 1 to 8 carbon atoms, or 1 to 6 carbon atoms, or 1 to 3 carbon atoms. For example, C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “propyl” includes n-propyl and isopropyl; and “butyl” includes n-butyl, sec-butyl, isobutyl and t-butyl. Examples of alkyl groups include, but are not limited to, methyl, ethyl, propyl, isopropyl, n-butyl, sec-butyl, tert-butyl, pentyl, 2-pentyl, 3-pentyl, isopentyl, neopentyl, hexyl, 2-hexyl, 3-hexyl, and 3-methylpentyl.

When a range of values is given (e.g., C₁₋₆ alkyl), each value within the range as well as all intervening ranges are included. For example, “C₁₋₆ alkyl” includes C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

“Cycloalkyl” indicates a non-aromatic, fully saturated carbocyclic ring having the indicated number of carbon atoms, for example, 3 to 10, or 3 to 8, or 3 to 6 ring carbon atoms. Cycloalkyl groups may be monocyclic or polycyclic (e.g., bicyclic, tricyclic). Examples of cycloalkyl groups include cyclopropyl, cyclobutyl, cyclopentyl, and cyclohexyl, as well as polycyclic spiro, fused, bridged and caged ring groups (e.g., norbornane, bicyclo[2.2.2]octane). In addition, one ring of a polycyclic cycloalkyl group may be aromatic, provided the polycyclic cycloalkyl group is bound to the parent structure via a non-aromatic carbon. For example, a 1,2,3,4-tetrahydronaphthalen-1-yl group (wherein the moiety is bound to the parent structure via a non-aromatic carbon atom) is a cycloalkyl group, while 1,2,3,4-tetrahydronaphthalen-5-yl (wherein the moiety is bound to the parent structure via an aromatic carbon atom) is not considered a cycloalkyl group. Examples of polycyclic cycloalkyl groups consisting of a cycloalkyl group fused to an aromatic ring are described below.

“Cycloalkenyl” indicates a non-aromatic carbocyclic ring, containing the indicated number of carbon atoms (e.g., 3 to 10, or 3 to 8, or 3 to 6 ring carbon atoms) and at least one carbon-carbon double bond. Cycloalkenyl groups may be monocyclic or polycyclic (e.g., bicyclic, tricyclic). Examples of cycloalkenyl groups include cyclopropenyl, cyclobutenyl, cyclopentenyl, cyclopentadienyl, and cyclohexenyl, as well as bridged and caged ring groups (e.g., bicyclo[2.2.2]octene). In addition, one ring of a polycyclic cycloalkenyl group may be aromatic, provided the polycyclic alkenyl group is bound to the parent structure via a non-aromatic carbon atom. For example, inden-1-yl (wherein the moiety is bound to the parent structure via a non-aromatic carbon atom) is considered a cycloalkenyl group, while inden-4-yl (wherein the moiety is bound to the parent structure via an aromatic carbon atom) is not considered a cycloalkenyl group. Examples of polycyclic cycloalkenyl groups consisting of a cycloalkenyl group fused to an aromatic ring are described below.

“Aryl” indicates an aromatic carbocyclic ring having the indicated number of carbon atoms, for example, 6 to 12 or 6 to 10 carbon atoms. Aryl groups may be monocyclic or polycyclic (e.g., bicyclic, tricyclic). In some instances, both rings of a polycyclic aryl group are aromatic (e.g., naphthyl). In other instances, polycyclic aryl groups may include a non-aromatic ring fused to an aromatic ring, provided the polycyclic aryl group is bound to the parent structure via an atom in the aromatic ring. Thus, a 1,2,3,4-tetrahydronaphthalen-5-yl group (wherein the moiety is bound to the parent structure via an aromatic carbon atom) is considered an aryl group, while 1,2,3,4-tetrahydronaphthalen-1-yl (wherein the moiety is bound to the parent structure via a non-aromatic carbon atom) is not considered an aryl group. Similarly, a 1,2,3,4-tetrahydroquinolin-8-yl group (wherein the moiety is bound to the parent structure via an aromatic carbon atom) is considered an aryl group, while 1,2,3,4-tetrahydroquinolin-1-yl group (wherein the moiety is bound to the parent structure via a non-aromatic nitrogen atom) is not considered an aryl group. However, the term “aryl” does not encompass or overlap with “heteroaryl,” as defined herein, regardless of the point of attachment (e.g., both quinolin-5-yl and quinolin-2-yl are heteroaryl groups). In some instances, aryl is phenyl or naphthyl. In certain instances, aryl is phenyl. Additional examples of aryl groups comprising an aromatic carbon ring fused to a non-aromatic ring are described below.

“Heteroaryl” indicates an aromatic ring containing the indicated number of atoms (e.g., 5 to 12, or 5 to 10 membered heteroaryl) made up of one or more heteroatoms (e.g., 1, 2, 3 or 4 heteroatoms) selected from N, O and S and with the remaining ring atoms being carbon. Heteroaryl groups do not contain adjacent S and O atoms. In some embodiments, the total number of S and O atoms in the heteroaryl group is not more than 2. In some embodiments, the total number of S and O atoms in the heteroaryl group is not more than 1. Unless otherwise indicated, heteroaryl groups may be bound to the parent structure by a carbon or nitrogen atom, as valency permits. For example, “pyridyl” includes 2-pyridyl, 3-pyridyl and 4-pyridyl groups, and “pyrrolyl” includes 1-pyrrolyl, 2-pyrrolyl and 3-pyrrolyl groups.

In some instances, a heteroaryl group is monocyclic. Examples include pyrrole, pyrazole, imidazole, triazole (e.g., 1,2,3-triazole, 1,2,4-triazole, 1,2,4-triazole), tetrazole, furan, isoxazole, oxazole, oxadiazole (e.g., 1,2,3-oxadiazole, 1,2,4-oxadiazole, 1,3,4-oxadiazole), thiophene, isothiazole, thiazole, thiadiazole (e.g., 1,2,3-thiadiazole, 1,2,4-thiadiazole, 1,3,4-thiadiazole), pyridine, pyridazine, pyrimidine, pyrazine, triazine (e.g., 1,2,4-triazine, 1,3,5-triazine) and tetrazine.

In some instances, both rings of a polycyclic heteroaryl group are aromatic. Examples include indole, isoindole, indazole, benzoimidazole, benzotriazole, benzofuran, benzoxazole, benzoisoxazole, benzoxadiazole, benzothiophene, benzothiazole, benzoisothiazole, benzothiadiazole, 1H-pyrrolo[2,3-b]pyridine, 1H-pyrazolo[3,4-b]pyridine, 3H-imidazo[4,5-b]pyridine, 3H-[1,2,3]triazolo[4,5-b]pyridine, 1H-pyrrolo[3,2-b]pyridine, 1H-pyrazolo[4,3-b]pyridine, 1H-imidazo[4,5-b]pyridine, 1H-[1,2,3]triazolo[4,5-b]pyri dine, 1H-pyrrolo[2,3-c]pyridine, 1H-pyrazolo[3,4-c]pyridine, 3H-imidazo[4,5-c]pyridine, 3H-[1,2,3]triazolo[4,5-c]pyridine, 1H-pyrrolo[3,2-c]pyridine, 1H-pyrazolo[4,3-c]pyridine, 1H-imidazo[4,5-c]pyridine, 1H-[1,2,3]triazolo[4,5-c]pyridine, furo[2,3-b]pyridine, oxazolo[5,4-b]pyridine, isoxazolo[5,4-b]pyridine, [1,2,3]oxadiazolo[5,4-b]pyridine, furo[3,2-b]pyridine, oxazolo[4,5-b]pyridine, isoxazolo[4,5-b]pyridine, [1,2,3]oxadiazolo[4,5-b]pyridine, furo[2,3-c]pyridine, oxazolo[5,4-c]pyridine, isoxazolo[5,4-c]pyridine, [1,2,3]oxadiazolo[5,4-c]pyridine, furo[3,2-c]pyridine, oxazolo[4,5-c]pyridine, isoxazolo[4,5-c]pyri dine, [1,2,3]oxadiazolo[4,5-c]pyridine, thieno[2,3-b]pyridine, thiazolo[5,4-b]pyridine, isothiazolo[5,4-b]pyridine, [1,2,3]thiadiazolo[5,4-b]pyridine, thieno[3,2-b]pyridine, thiazolo[4,5-b]pyridine, isothiazolo[4,5-b]pyridine, [1,2,3]thiadiazolo[4,5-b]pyridine, thieno[2,3-c]pyridine, thiazolo[5,4-c]pyridine, isothiazolo[5,4-c]pyridine, [1,2,3]thiadiazolo[5,4-c]pyridine, thieno[3,2-c]pyridine, thiazolo[4,5-c]pyridine, isothiazolo[4,5-c]pyridine, [1,2,3]thiadiazolo[4,5-c]pyridine, quinoline, isoquinoline, cinnoline, quinazoline, quinoxaline, phthalazine, naphthyridine (e.g., 1,8-naphthyridine, 1,7-naphthyridine, 1,6-naphthyridine, 1,5-naphthyridine, 2,7-naphthyridine, 2,6-naphthyridine), imidazo[1,2-a]pyridine, 1H-pyrazolo[3,4-d]thiazole, 1H-pyrazolo[4,3-d]thiazole and imidazo[2,1-b]thiazole.

In other instances, polycyclic heteroaryl groups may include a non-aromatic ring (e.g., cycloalkyl, cycloalkenyl, heterocycloalkyl, heterocycloalkenyl) fused to a heteroaryl ring, provided the polycyclic heteroaryl group is bound to the parent structure via an atom in the aromatic ring. For example, a 4,5,6,7-tetrahydrobenzo[d]thiazol-2-yl group (wherein the moiety is bound to the parent structure via an aromatic carbon atom) is considered a heteroaryl group, while 4,5,6,7-tetrahydrobenzo[d]thiazol-5-yl (wherein the moiety is bound to the parent structure via a non-aromatic carbon atom) is not considered a heteroaryl group. Examples of polycyclic heteroaryl groups consisting of a heteroaryl ring fused to a non-aromatic ring are described below.

“Heterocycloalkyl” indicates a non-aromatic, fully saturated ring having the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon. Heterocycloalkyl groups may be monocyclic or polycyclic (e.g., bicyclic, tricyclic). Examples of heterocycloalkyl groups include oxiranyl, aziridinyl, azetidinyl, pyrrolidinyl, imidazolidinyl, pyrazolidinyl, piperidinyl, piperazinyl, morpholinyl and thiomorpholinyl, as well as polycyclic spiro, fused, bridged and caged ring groups. Examples include thiomorpholine S-oxide and thiomorpholine S,S-dioxide. In addition, one ring of a polycyclic heterocycloalkyl group may be aromatic (e.g., aryl or heteroaryl), provided the polycyclic heterocycloalkyl group is bound to the parent structure via a non-aromatic carbon or nitrogen atom. For example, a 1,2,3,4-tetrahydroquinolin-1-yl group (wherein the moiety is bound to the parent structure via a non-aromatic nitrogen atom) is considered a heterocycloalkyl group, while 1,2,3,4-tetrahydroquinolin-8-yl group (wherein the moiety is bound to the parent structure via an aromatic carbon atom) is not considered a heterocycloalkyl group. Examples of polycyclic heterocycloalkyl groups consisting of a heterocycloalkyl group fused to an aromatic ring are described below.

“Heterocycloalkenyl” indicates a non-aromatic ring having the indicated number of atoms (e.g., 3 to 10, or 3 to 7, membered heterocycloalkyl) made up of one or more heteroatoms (e.g., 1, 2, 3 or 4 heteroatoms) selected from N, O and S and with the remaining ring atoms being carbon, and at least one double bond derived by the removal of one molecule of hydrogen from adjacent carbon atoms, adjacent nitrogen atoms, or adjacent carbon and nitrogen atoms of the corresponding heterocycloalkyl. Heterocycloalkenyl groups may be monocyclic or polycyclic (e.g., bicyclic, tricyclic). Examples of heterocycloalkenyl groups include dihydrofuranyl (e.g., 2,3-dihydrofuranyl, 2,5-dihydrofuranyl), dihydrothiophenyl (e.g., 2,3-dihydrothiophenyl, 2,5-dihydrothiophenyl), dihydropyrrolyl (e.g., 2,3-dihydro-1H-pyrrolyl, 2,5-dihydro-1H-pyrrolyl), dihydroimidazolyl (e.g., 2,3-dihydro-1H-imidazolyl, 4,5-dihydro-1H-imidazolyl), pyranyl, dihydropyranyl (e.g., 3,4-dihydro-2H-pyranyl, 3,6-dihydro-2H-pyranyl), tetrahydropyridinyl (e.g., 1,2,3,4-tetrahydropyridinyl, 1,2,3,6-tetrahydropyridinyl) and dihydropyridine (e.g., 1,2-dihydropyridine, 1,4-dihydropyridine). In addition, one ring of a polycyclic heterocycloalkenyl group may be aromatic (e.g., aryl or heteroaryl), provided the polycyclic heterocycloalkenyl group is bound to the parent structure via a non-aromatic carbon or nitrogen atom. For example, a 1,2-dihydroquinolin-1-yl group (wherein the moiety is bound to the parent structure via a non-aromatic nitrogen atom) is considered a heterocycloalkenyl group, while 1,2-dihydroquinolin-8-yl group (wherein the moiety is bound to the parent structure via an aromatic carbon atom) is not considered a heterocycloalkenyl group. Examples of polycyclic heterocycloalkenyl groups consisting of a heterocycloalkenyl group fused to an aromatic ring are described below.

Examples of polycyclic rings consisting of an aromatic ring (e.g., aryl or heteroaryl) fused to a non-aromatic ring (e.g., cycloalkyl, cycloalkenyl, heterocycloalkyl, heterocycloalkenyl) include indenyl, 2,3-dihydro-1H-indenyl, 1,2,3,4-tetrahydronaphthalenyl, benzo[1,3]dioxolyl, tetrahydroquinolinyl, 2,3-dihydrobenzo[1,4]dioxinyl, indolinyl, isoindolinyl, 2,3-dihydro-1H-indazolyl, 2,3-dihydro-1H-benzo[d]imidazolyl, 2,3-dihydrobenzofuranyl, 1,3-dihydroisobenzofuranyl, 1,3-dihydrobenzo[c]isoxazolyl, 2,3-dihydrobenzo[d]isoxazolyl, 2,3-dihydrobenzo[d]oxazolyl, 2,3-dihydrobenzo[b]thiophenyl, 1,3-dihydrobenzo[c]thiophenyl, 1,3-dihydrobenzo[c]isothiazolyl, 2,3-dihydrobenzo[d]isothiazolyl, 2,3-dihydrobenzo[d]thiazolyl, 5,6-dihydro-4H-cyclopenta[d]thiazolyl, 4,5,6,7-tetrahydrobenzo[d]thiazolyl, 5,6-dihydro-4H-pyrrolo[3,4-d]thiazolyl, 4,5,6,7-tetrahydrothiazolo[5,4-c]pyridinyl, indolin-2-one, indolin-3-one, isoindolin-1-one, 1,2-dihydroindazol-3-one, 1H-benzo[d]imidazol-2(3H)-one, benzofuran-2(3H)-one, benzofuran-3(2H)-one, isobenzofuran-1(3H)-one, benzo[c]isoxazol-3(1H)-one, benzo[d]isoxazol-3(2H)-one, benzo[d]oxazol-2(3H)-one, benzo[b]thiophen-2(3H)-one, benzo[b]thiophen-3(2H)-one, benzo[c]thiophen-1(3H)-one, benzo[c]isothiazol-3(1H)-one, benzo[d]isothiazol-3(2H)-one, benzo[d]thiazol-2(3H)-one, 4,5-dihydropyrrolo[3,4-d]thiazol-6-one, 1,2-dihydropyrazolo[3,4-d]thiazol-3-one, quinolin-4(3H)-one, quinazolin-4(3H)-one, quinazoline-2,4(1H,3H)-dione, quinoxalin-2(1H)-one, quinoxaline-2,3(1H,4H)-dione, cinnolin-4(3H)-one, pyridin-2(1H)-one, pyrimidin-2(1H)-one, pyrimidin-4(3H)-one, pyridazin-3(2H)-one, 1H-pyrrolo[3,2-b]pyridin-2(3H)-one, 1H-pyrrolo[3,2-c]pyridin-2(3H)-one, 1H-pyrrolo[2,3-c]pyridin-2(3H)-one, 1H-pyrrolo[2,3-b]pyridin-2(3H)-one, 1,2-dihydropyrazolo[3,4-d]thiazol-3-one and 4,5-dihydropyrrolo[3,4-d]thiazol-6-one. As discussed herein, whether each ring is considered an aryl, heteroaryl, cycloalkyl, cycloalkenyl, heterocycloalkyl or heterocycloalkenyl group is determined by the atom through which the moiety is bound to the parent structure.

“Halogen” or “halo” refers to fluoro, chloro, bromo or iodo.

“Haloalkyl” refers to alkyl substituted with one or more halogen. A haloalkyl group may have a halogen substituent at any valence-permitted location on the alkyl and may have any number of halogen substituents ranging from one to the maximum valence-permitted number. Particular haloalkyl groups have 1, 2, or 3 halogen substituents. Examples of haloalkyl groups include, but are not limited to, —CH₂F, —CHF₂, —CF₃, —CH₂CH₂F, —CH₂CHF₂, —CH₂CF₃, —CH₂Cl, —CHCl₂, —CCl₃, —CH₂CH₂Cl, —CH₂CHCl₂, —CH₂CCl₃.

Unless otherwise indicated, compounds disclosed and/or described herein include all possible enantiomers, diastereomers, meso isomers and other stereoisomeric forms, including racemic mixtures, optically pure forms and intermediate mixtures thereof. Enantiomers, diastereomers, meso isomers and other stereoisomeric forms can be prepared using chiral synthons or chiral reagents, or resolved using conventional techniques. Unless specified otherwise, when the compounds disclosed and/or described herein contain olefinic double bonds or other centers of geometric asymmetry, it is intended that the compounds include both E and Z isomers. When the compounds described herein contain moieties capable of tautomerization, and unless specified otherwise, it is intended that the compounds include all possible tautomers.

“Protecting group” has the meaning conventionally associated with it in organic synthesis, i.e., a group that selectively blocks one or more reactive sites in a multifunctional compound such that a chemical reaction can be carried out selectively on another unprotected reactive site, and such that the group can readily be removed after the selective reaction is complete. A variety of protecting groups are disclosed, for example, in T. H. Greene and P. G. M. Wuts, Protective Groups in Organic Synthesis, Third Edition, John Wiley & Sons, New York (1999). For example, a “hydroxy protected form” contains at least one hydroxy group protected with a hydroxy protecting group. Likewise, amines and other reactive groups may similarly be protected.

The term “pharmaceutically acceptable salt” refers to a salt of any of the compounds herein which are known to be non-toxic and are commonly used in the pharmaceutical literature. In some embodiments, the pharmaceutically acceptable salt of a compound retains the biological effectiveness of the compounds described herein and are not biologically or otherwise undesirable. Examples of pharmaceutically acceptable salts can be found in Berge et al., Pharmaceutical Salts, J. Pharmaceutical Sciences, January 1977, 66(1), 1-19. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, lactic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, 2-hydroxyethylsulfonic acid, p-toluenesulfonic acid, stearic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines; substituted amines including naturally occurring substituted amines; cyclic amines; and basic ion exchange resins. Examples of organic bases include isopropylamine, trimethylamine, diethylamine, triethylamine, tripropylamine, and ethanolamine. In some embodiments, the pharmaceutically acceptable base addition salt is selected from ammonium, potassium, sodium, calcium, and magnesium salts.

If the compound described herein is obtained as an acid addition salt, the free base can be obtained by basifying a solution of the acid salt. Conversely, if the compound is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds (see, e.g., Berge et al., Pharmaceutical Salts, J. Pharmaceutical Sciences, January 1977, 66(1), 1-19). Those skilled in the art will recognize various synthetic methodologies that may be used to prepare pharmaceutically acceptable addition salts.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

The term “substituted” means that the specified group or moiety bears one or more substituents including, but not limited to, substituents such as alkoxy, acyl, acyloxy, alkoxycarbonyl, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, alkyl, alkenyl, alkynyl, heterocycloalkyl, heterocycloalkenyl, aralkyl, aminosulfonyl, sulfonylamino, sulfonyl, oxo and the like. The term “unsubstituted” means that the specified group bears no substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The compounds disclosed and/or described herein can be enriched isotopic forms, e.g., enriched in the content of ²H, ³H, ¹¹C, ¹³C and/or ¹⁴C. In one embodiment, the compound contains at least one deuterium atom. Such deuterated forms can be made, for example, by the procedure described in U.S. Pat. Nos. 5,846,514 and 6,334,997. Such deuterated compounds may improve the efficacy and increase the duration of action of compounds disclosed and/or described herein. Deuterium substituted compounds can be synthesized using various methods, such as those described in: Dean, D., Recent Advances in the Synthesis and Applications of Radiolabeled Compounds for Drug Discovery and Development, Curr. Pharm. Des., 2000; 6(10); Kabalka, G. et al., The Synthesis of Radiolabeled Compounds via Organometallic Intermediates, Tetrahedron, 1989, 45(21), 6601-21; and Evans, E., Synthesis of radiolabeled compounds, J. Radioanal. Chem., 1981, 64(1-2), 9-32.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in pharmaceutical compositions is contemplated. Supplementary active ingredients can also be incorporated into the pharmaceutical compositions.

The terms “patient,” “individual,” and “subject” refer to an animal, such as a mammal, bird, or fish. In some embodiments, the patient or subject is a mammal. Mammals include, for example, mice, rats, dogs, cats, pigs, sheep, horses, cows and humans. In some embodiments, the patient, individual, or subject is a human, for example a human that has been or will be the object of treatment, observation or experiment. The compounds, compositions and methods described herein can be useful in both human therapy and veterinary applications.

The term “therapeutically effective amount” or “effective amount” refers to that amount of a compound disclosed and/or described herein that is sufficient to affect treatment, as defined herein, when administered to a patient in need of such treatment. A therapeutically effective amount of a compound may be an amount sufficient to treat a disease responsive to modulation (e.g., inhibition) of KIF18a. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

“Treatment” (and related terms, such as “treat,” “treated,” “treating”) includes one or more of: inhibiting a disease or disorder; slowing or arresting the development of clinical symptoms of a disease or disorder; and/or relieving a disease or disorder (i.e., causing relief from or regression of clinical symptoms). The term covers both complete and partial reduction of the condition or disorder, and complete or partial reduction of clinical symptoms of a disease or disorder. Thus, compounds described and/or disclosed herein may prevent an existing disease or disorder from worsening, assist in the management of the disease or disorder, or reduce or eliminate the disease or disorder.

It is understood that embodiments described herein as “comprising” include “consisting of” and “consisting essentially of” embodiments.

### Compounds

Compounds and salts thereof (such as pharmaceutically acceptable salts) are detailed herein, including in the Brief Summary and in the appended claims. Also provided are the use of all of the compounds described herein, including any and all stereoisomers, including geometric isomers (cis/trans), F/Z isomers, enantiomers, diastereomers, and mixtures thereof in any ratio including racemic mixtures, salts and solvates of the compounds described herein, as well as methods of making such compounds. Any compound described herein may also be referred to as a drug.

In one aspect, provided are compounds of Formula (I):

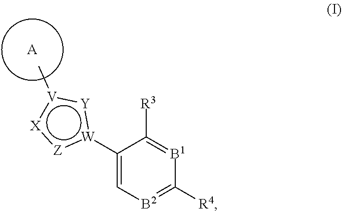

or a pharmaceutically acceptable salt thereof, wherein:
X and Z are independently O, N, or CH;
Y is NH, N, or CH;
V and W are independently N or C;
   wherein at least one of X and Z is N or Y is NH;
Ring A is

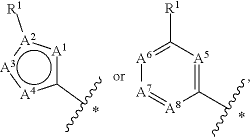

wherein
one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O;
wherein “*” indicates the point of attachment to V;
B¹ and B² are each independently N, CH or CR^{B}, wherein R^{B} is halogen;
R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 10-membered heterocycloalkyl;
R³ is piperidinyl, pyrrolidinyl, or azepanyl, wherein the piperidinyl, the pyrrolidinyl, or the azepanyl, is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or azepanyl, or
   wherein the piperidinyl, pyrrolidinyl, or azepanyl are optionally substituted with a C₁₋₂ alkylene to form a bridged piperidinyl, pyrrolidinyl, or azepanyl ring system,
   wherein the piperidinyl, the pyrrolidinyl, the azepanyl, or the spirocyclic, fused, or bridged bicyclic ring system formed by the C₃₋₁₀ cycloalkyl, 3- to 10-membered heterocycloalkyl, or C₁₋₂ alkylene with piperidinyl, pyrrolidinyl, or azepanyl is optionally substituted with one or more substituents independently selected from the group consisting of C₁-C₃ alkyl, C₁-C₃ haloalkyl, and halo;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH.

In some embodiments, when Y is N, then R⁴ is not H. In some embodiments, when X is N, Y is N, and Z is O, then R⁴ is not H. In some embodiments, when Y is N, then R³ is

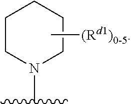

In some embodiments, when X is N, Y is N, and Z is O, then R³ is

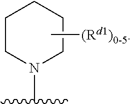

In some embodiments, each R^{d1} is independently selected from the group consisting of C₁-C₃ alkyl, C₁-C₃ haloalkyl, and halo. In some embodiments, two R¹ are taken together to form a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl. In some embodiments, two R^{d1} are taken together to form a C₁₋₂ alkylene, wherein the C₁₋₂ alkylene forms a bridged piperidinyl ring system. In some embodiments, the spirocyclic, fused, or bridged bicyclic ring system formed by the C₃₋₁₀ cycloalkyl, 3- to 10-membered heterocycloalkyl, or C₁₋₂ alkylene with the piperidinyl is optionally substituted with one or more substituents independently selected from the group consisting of C₁-C₃ alkyl, C₁-C₃ haloalkyl, and halo. In some embodiments, when Y is N, then R³ is

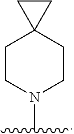 [SMILES: *N1CCC2(CC1)CC2]

In some embodiments, when X is N, Y is N, and Z is O, then R³ is

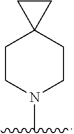 [SMILES: *N1CCC2(CC1)CC2]

In some embodiments, the compound is not 3-(5-methylfuran-2-yl)-5-(2-(piperidin-4-yl)pyridin-3-yl)-1,2,4-oxadiazole. In some embodiments, the compound is not 3-(3-methyl-5,6,7,8-tetrahydro-2,7-naphthyridin-4-yl)-5-(2-(pyrrolidin-1-yl)phenyl)-1,2,4-oxadiazole. In some embodiments, the compound is not 3-(5-methylfuran-2-yl)-5-(2-(piperidin-4-yl)pyridin-3-yl)-1,2,4-oxadiazole or 3-(3-methyl-5,6,7,8-tetrahydro-2,7-naphthyridin-4-yl)-5-(2-(pyrrolidin-1-yl)phenyl)-1,2,4-oxadiazole.

In some embodiments, the compound is not a salt of 3-(5-methylfuran-2-yl)-5-(2-(piperidin-4-yl)pyridin-3-yl)-1,2,4-oxadiazole. In some embodiments, the compound is not a salt of 3-(3-methyl-5,6,7,8-tetrahydro-2,7-naphthyridin-4-yl)-5-(2-(pyrrolidin-1-yl)phenyl)-1,2,4-oxadiazole. In some embodiments, the compound is not a salt of 3-(5-methylfuran-2-yl)-5-(2-(piperidin-4-yl)pyridin-3-yl)-1,2,4-oxadiazole or 3-(3-methyl-5,6,7,8-tetrahydro-2,7-naphthyridin-4-yl)-5-(2-(pyrrolidin-1-yl)phenyl)-1,2,4-oxadiazole.

In some embodiments, the compound of Formula (I) is a compound of Formula (II):

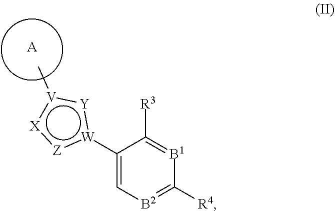

or a pharmaceutically acceptable salt thereof.

In one aspect, provided are compounds of Formula (II):

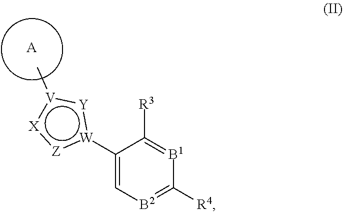

or a pharmaceutically acceptable salt thereof, wherein:
X and Z are independently O, N, or CH;
Y is NH or CH;
V and W are independently N or C;
   wherein at least one of X and Z is N or Y is NH;
Ring A is

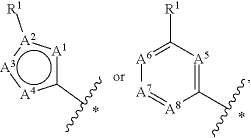

wherein
one or two of A¹, A³, and A⁴ are independently N, O, or S, and the remaining one or two of A¹, A³, and A⁴ are independently CH or CR²;
A² is N or C;
A⁵-A⁸ are independently CH, CR² or N, wherein at least two of A⁵, A⁶, A⁷, and A⁸ are CH or CR², and the remaining one or two of A⁵, A⁶, A⁷, and A⁸, if present, are N;
wherein “*” indicates the point of attachment to V;
B¹ and B² are each independently N or CH;
R¹ is C₁₋₆ alkyl, C₃₋₆ cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R¹ is optionally substituted with one or more halogens;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein R^{b1} and R^{b2} are independently optionally substituted with C₁—C; alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 6-membered heterocycloalkyl;
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or the azepanyl, and
   wherein the piperidinyl, the pyrrolidinyl, the azepanyl or the spirocyclic or fused bicyclic ring system formed by the C₃₋₁₀ cycloalkyl or 3- to 10-membered heterocycloalkyl with piperidinyl, pyrrolidinyl, or azepanyl is optionally substituted with one or more substituents independently selected from the group consisting of C₁-C₃ alkyl and C₁-C₃ haloalkyl;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

In some embodiments, X and Z are independently O, N, or CH, and Y is NH, N, or CH, wherein at least one of X and Z is N or Y is NH.

In some embodiments, X is O, N, or CH. In some embodiments, X is O or N. In other embodiments X is O or CH. In still other embodiments, X is N or CH. In some embodiments X is N. In other embodiments, X is O. In still other embodiments, X is CH.

In some embodiments, Z is O, N, or CH. In some embodiments, Z is O or N. In other embodiments Z is O or CH. In still other embodiments, Z is N or CH. In some embodiments Z is N. In other embodiments, Z is O. In still other embodiments, Z is CH.

In some embodiments, Y is NH or CH. In some embodiments, Y is NH. In other embodiments, Y is CH.

In some embodiments, V is N or C. In some embodiments, V is N. In other embodiments, V is C.

In some embodiments, W is N or C. In some embodiments, W is N. In other embodiments, W is C.

In some embodiments, the ring

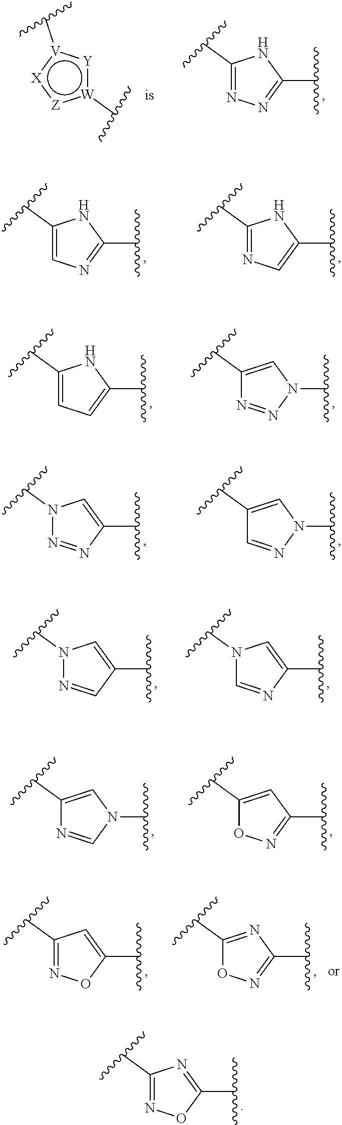

In some embodiments, the ring

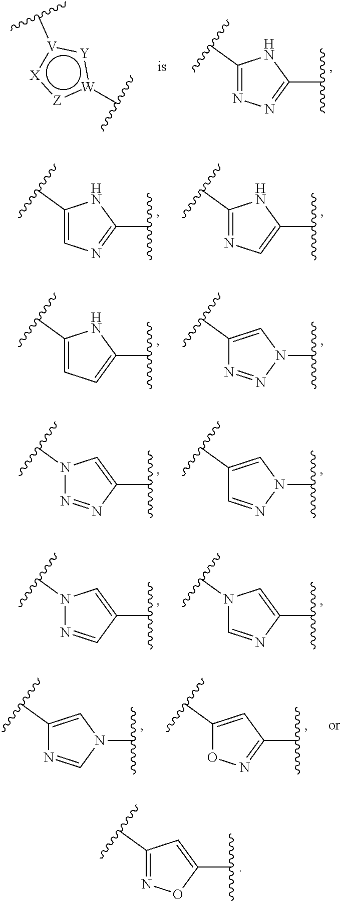

In certain embodiments, the ring

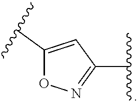 [SMILES: *c1cc(*)on1]

In some embodiments, the ring

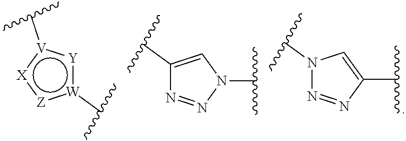

In certain embodiments, the ring

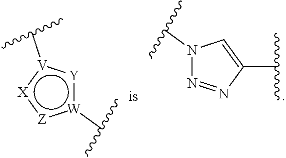

In certain embodiments, the ring

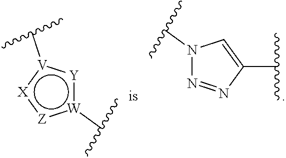

It should be recognized that for any embodiments wherein Y is NH, the ring

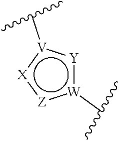

can encompass any valid tautomers thereof, including those, for example wherein Y is N, and X or Z is NH. For example, in some embodiments, the ring

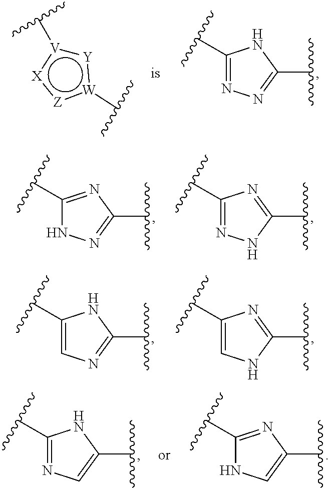

In some embodiments of any of the foregoing, the compound of Formula (I) is a compound of Formula (I-a):

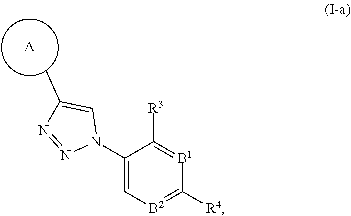

or a pharmaceutically acceptable salt thereof, wherein:
Ring A is

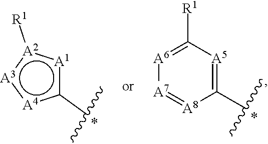

wherein
one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O;
wherein “*” indicates the point of attachment to V;
B¹ and B² are each independently N, CH or CR^{B}, wherein R^{B} is halogen;
R¹ is C₁₋₆, alkyl, C₃₋₄ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 10-membered heterocycloalkyl;
R³ is piperidinyl, pyrrolidinyl, or azepanyl, wherein the piperidinyl, the pyrrolidinyl, or the azepanyl, is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or azepanyl, or
   wherein the piperidinyl, pyrrolidinyl, or azepanyl are optionally substituted with a C₁₋₂ alkylene to form a bridged piperidinyl, pyrrolidinyl, or azepanyl ring system,
   wherein the piperidinyl, the pyrrolidinyl, the azepanyl, or the spirocyclic, fused, or bridged bicyclic ring system formed by the C₃₋₁₀ cycloalkyl, 3- to 10-membered heterocycloalkyl, or C₁₋₂ alkylene with piperidinyl, pyrrolidinyl, or azepanyl is optionally substituted with one or more substituents independently selected from the group consisting of C₁-C₃ alkyl, C₁-C₃ haloalkyl, and halo;
R⁴ is H, halo, cyano, —OH, —NO₂, —C(O)NR^{c1}R^{c2}, —N^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH.

In some embodiments of any of the foregoing, the compound of Formula (I) is a compound of Formula (I-b):

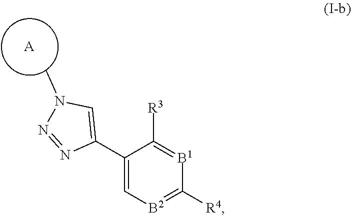

or a pharmaceutically acceptable salt thereof, wherein:
Ring A is

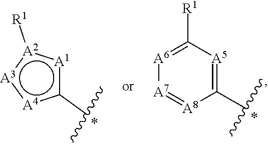

wherein
one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O;
wherein “*” indicates the point of attachment to V;
B¹ and B² are each independently N, CH or CR^{B}, wherein R^{B} is halogen;
R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 10-membered heterocycloalkyl;
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or azepanyl, or
   wherein the piperidinyl, pyrrolidinyl, or azepanyl are optionally substituted with a C₁₋₂ alkylene to form a bridged piperidinyl, pyrrolidinyl, or azepanyl ring system,
   wherein the piperidinyl, the pyrrolidinyl, the azepanyl, or the spirocyclic, fused, or bridged bicyclic ring system formed by the C₃₋₁₀ cycloalkyl, 3- to 10-membered heterocycloalkyl, or C₁₋₂ alkylene with piperidinyl, pyrrolidinyl, or azepanyl, is optionally substituted with one or more substituents independently selected from the group consisting of C₁-C₃ alkyl, C₁-C₃ haloalkyl, and halo;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH.

In some embodiments of any of the foregoing, the compound of Formula (I) is a compound of Formula (I-c):

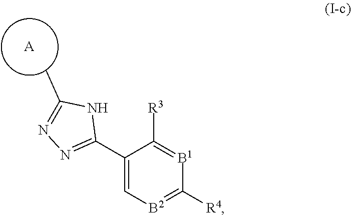

or a pharmaceutically acceptable salt thereof, wherein:
Ring A is

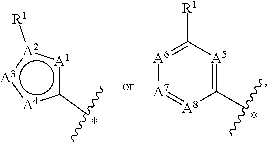

wherein
one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O;
wherein “*” indicates the point of attachment to V;
B¹ and B² are each independently N, CH or CR^{B}, wherein R^{B} is halogen;
R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 10-membered heterocycloalkyl;
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or azepanyl, or
   wherein the piperidinyl, pyrrolidinyl, or azepanyl are optionally substituted with a C₁₋₂ alkylene to form a bridged piperidinyl, pyrrolidinyl, or azepanyl ring system,
   wherein the piperidinyl, the pyrrolidinyl, the azepanyl, or the spirocyclic, fused, or bridged bicyclic ring system formed by the C₃₋₁₀ cycloalkyl, 3- to 10-membered heterocycloalkyl, or C₁₋₂ alkylene with piperidinyl, pyrrolidinyl, or azepanyl is optionally substituted with one or more substituents independently selected from the group consisting of C₁-C₃ alkyl, C₁-C₃ haloalkyl, and halo;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH.

In some embodiments, the compound of Formula (II) is a compound of Formula (II-a):

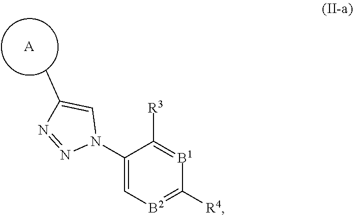

or a pharmaceutically acceptable salt thereof, wherein:
Ring A is

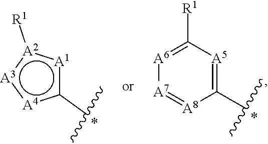

wherein
one or two of A¹, A³, and A⁴ are independently N, O, or S, and the remaining one or two of A¹, A³, and A⁴ are independently CH or CR²;
A² is N or C;
A⁵-A⁸ are independently CH, CR² or N, wherein at least two of A⁵, A⁶, A⁷, and A⁸ are CH or CR², and the remaining one or two of A⁵, A⁶, A⁷, and A⁸, if present, are N;
wherein “*” indicates the point of attachment to V;
B¹ and B² are each independently N or CH;
R¹ is C₁₋₆ alkyl, C₃₋₆ cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R¹ is optionally substituted with one or more halogens;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 6-membered heterocycloalkyl:
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrolidinyl, or the azepanyl, and wherein the piperidinyl, the pyrrolidinyl, the azepanyl or the spirocyclic or fused bicyclic ring system formed by the C₃₋₁₀ cycloalkyl or 3- to 10-membered heterocycloalkyl with piperidinyl, pyrrolidinyl, or azepanyl is optionally substituted with one or more substituents independently selected from the group consisting of C₁-C₃ alkyl and C₁-C₃ haloalkyl; R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

In some embodiments, the compound of Formula (II) is a compound of Formula (II-b):

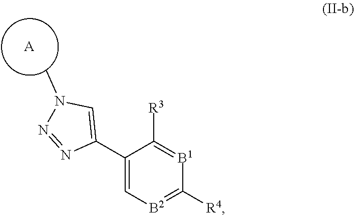

or a pharmaceutically acceptable salt thereof, wherein:
Ring A is

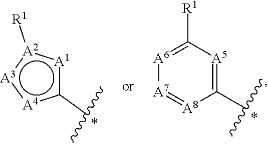

wherein
one or two of A¹, A³, and A⁴ are independently N, O, or S, and the remaining one or two of A¹, A³, and A⁴ are independently CH or CR²;
A² is N or C;
A⁵-A⁸ are independently CH, CR² or N, wherein at least two of A⁵, A⁶, A⁷, and A⁸ are CH or CR², and the remaining one or two of A¹, A⁶, A⁷, and A⁸, if present, are N;
wherein “*” indicates the point of attachment to V;
B¹ and B² are each independently N or CH;
R¹ is C₁₋₆ alkyl, C₃₋₆ cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R¹ is optionally substituted with one or more halogens;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 6-membered heterocycloalkyl;
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or the azepanyl, and
   wherein the piperidinyl, the pyrrolidinyl, the azepanyl or the spirocyclic or fused bicyclic ring system formed by the C₃₋₁₀ cycloalkyl or 3- to 10-membered heterocycloalkyl with piperidinyl, pyrrolidinyl, or azepanyl is optionally substituted with one or more substituents independently selected from the group consisting of C₁-C₃ alkyl and C₁-C₃ haloalkyl;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

In some embodiments, the compound of Formula (II) is a compound of Formula (I-c):

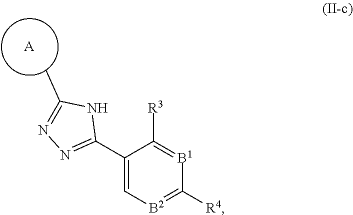

or a pharmaceutically acceptable salt thereof, wherein:
Ring A is

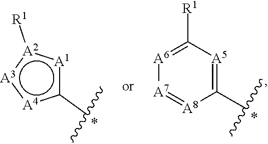

wherein
one or two of A¹, A³, and A⁴ are independently N, O, or S, and the remaining one or two of A¹, A³, and A⁴ are independently CH or CR²;
A² is N or C;
A⁵-A⁸ are independently CH, CR² or N, wherein at least two of A¹, A⁶, A⁷, and A⁸ are CH or CR², and the remaining one or two of A⁵, A⁶, A⁷, and A⁸, if present, are N,
wherein “*” indicates the point of attachment to V:
B¹ and B² are each independently N or CH;
R¹ is C₁₋₆ alkyl, C₃₋₆ cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R¹ is optionally substituted with one or more halogens;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R², wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 6-membered heterocycloalkyl:
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or the azepanyl, and
   wherein the piperidinyl, the pyrrolidinyl, the azepanyl or the spirocyclic or fused bicyclic ring system formed by the C₃₋₁₀ cycloalkyl or 3- to 10-membered heterocycloalkyl with piperidinyl, pyrrolidinyl, or azepanyl is optionally substituted with one or more substituents independently selected from the group consisting of C₁-C₃ alkyl and C₁-C₃ haloalkyl;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

In some embodiments, Ring A is

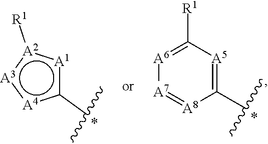

wherein one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl; A² is N or C; A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O; wherein “*” indicates the point of attachment to V.

In some embodiments, Ring A is

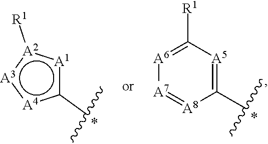

wherein one or two of A¹, A³, and A⁴ are independently N, O, or S, and the remaining one or two of A¹, A³, and A⁴ are independently CH or CR²; A² is N or C; A⁵-A⁸ are independently CH, CR² or N, wherein at least two of A⁵, A⁶, A⁷, and A⁸ are CH or CR², and the remaining one or two of A⁵, A⁶, A⁷, and A⁸, if present, are N; wherein “*” indicates the point of attachment to V.

In some embodiments, Ring A is

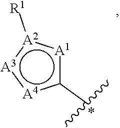

wherein one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl; A² is N or C. In certain embodiments, Ring A is

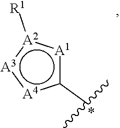

wherein one or two of A¹, A³, and A⁴ are independently N, O, or S, and the remaining one or two of A¹, A³, and A⁴ are independently CH or CR²; A² is N or C. In some embodiments, one of A¹, A³, and A⁴ are independently N, NR^{A1}, O, or S, and the remaining two of A¹, A³, and A⁴ are independently CH or CR². In certain embodiments, one of A¹, A³, and A⁴ are independently N, O, or S, and the remaining two of A¹, A³, and A⁴ are independently CH or CR². In other embodiments, two of A¹, A³, and A⁴ are independently N, NR^{A1}, O, or S, and the remaining one of A¹, A³, and A⁴ is independently CH or CR². In certain embodiments, two of A¹, A³, and A⁴ are independently N, O, or S, and the remaining one of A¹, A³, and A⁴ is independently CH or CR². In yet other embodiments, three of A¹, A³, and A⁴ are independently N, NR^{A1}, O, or S. In some embodiments, R^{A1}, if present, is H or C₁₋₃ alkyl. In certain embodiments, R^{A} is H or —CH₃.

In some embodiments, Ring A is

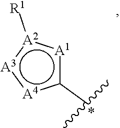

In some embodiments, Ring A is

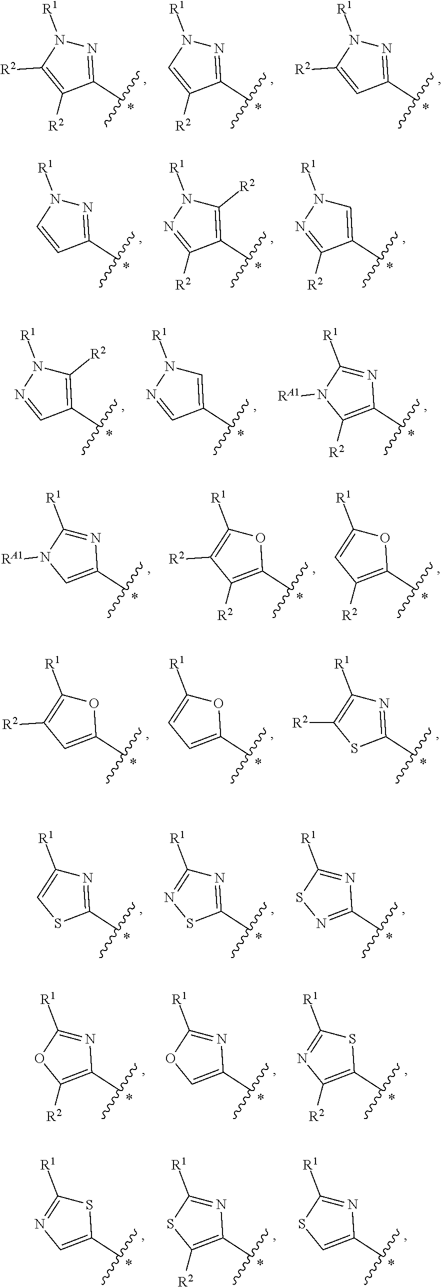

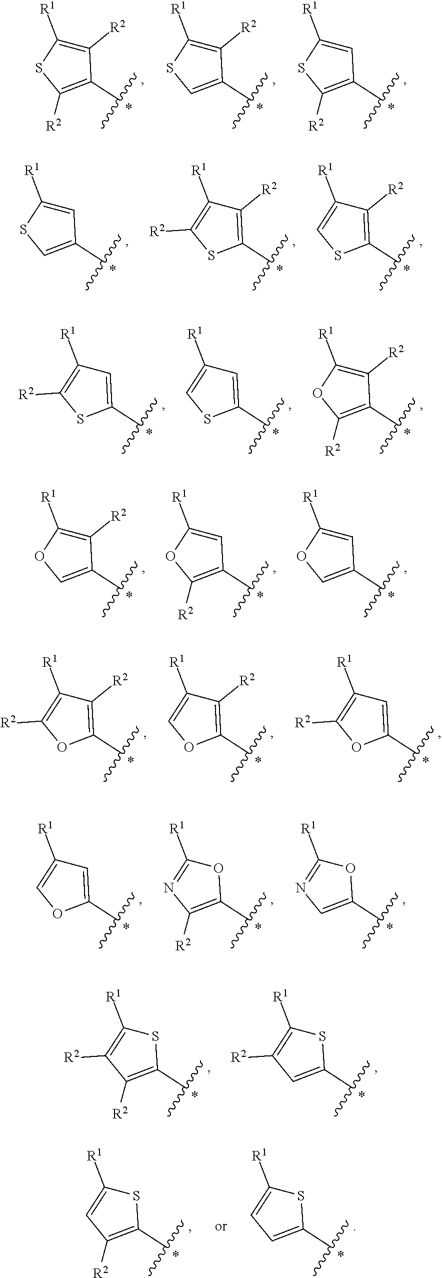

In some embodiments, Ring A is

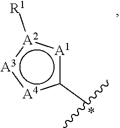

In some embodiments, Ring A is

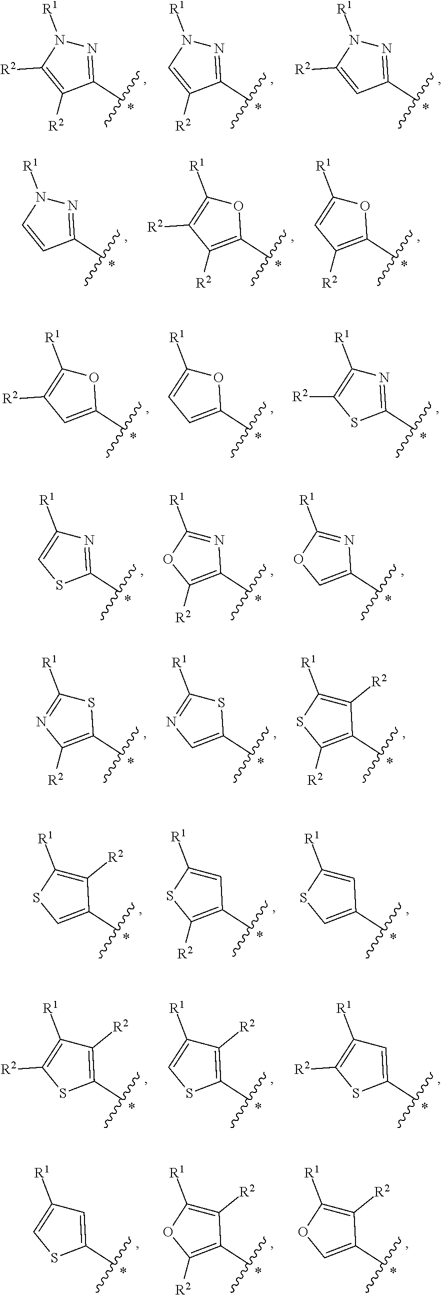

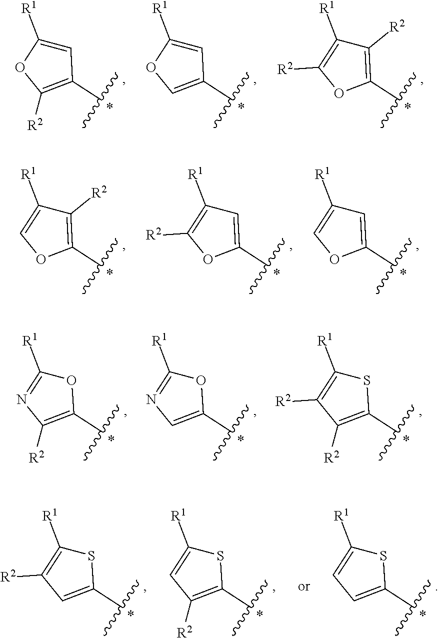

In some embodiments, Ring A is

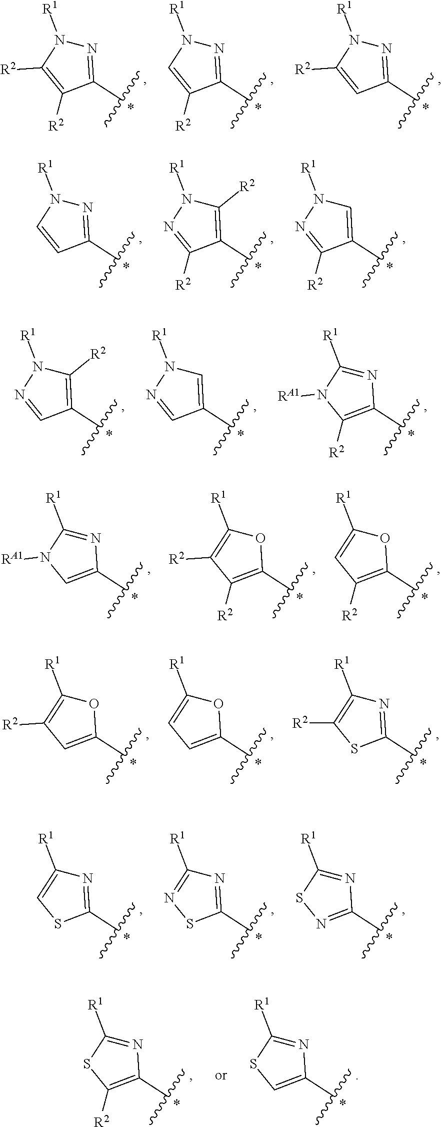

In some embodiments, Ring A is

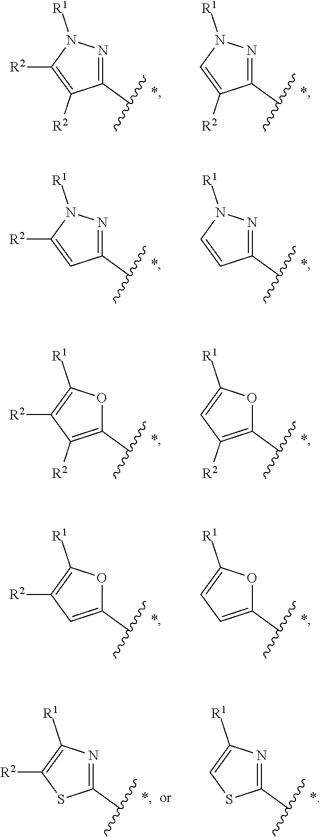

In certain embodiments, Ring A is

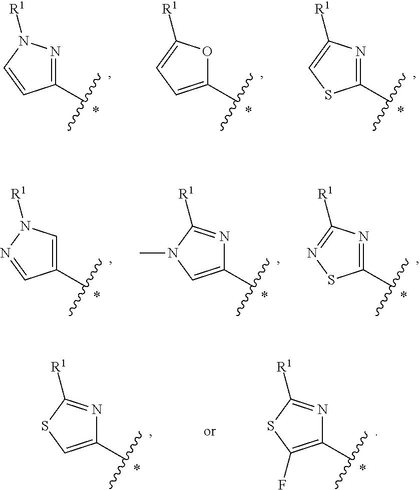

In certain embodiments, Ring A is

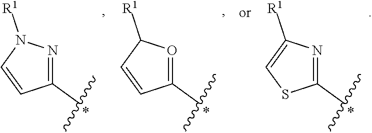

In some embodiments, Ring A is

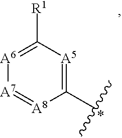

wherein A⁵-A⁸ are independently CH, CR², N, or NR^{A2}, wherein at least two of A⁵, A⁶, A⁷, and A⁸ are CH or CR², and the remaining one or two of A⁶, A⁶, A⁷, and A, if present are N or NR^{A2}, wherein R^{A2} is ═O. In some embodiments, Ring A is

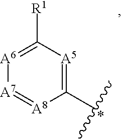

wherein A⁵-A⁸ are independently CH, CR² or N, wherein at least two of A⁵, A⁶, A⁷, and A⁸ are CH or CR², and the remaining one or two of A⁵, A⁶, A⁷, and A⁸, if present, are N. In some embodiments, two of A⁵, A⁶, A⁷, and A⁸ are CH or CR², and the remaining two of A⁵, A⁶, A⁷, and A⁸ are N or NR^{A2}, wherein R^{A2} is ═O. In certain embodiments, two of A⁵, A⁶, A⁷, and A⁸ are CH or CR², and the remaining two of A⁵, A⁶, A⁷, and A⁸ are N. In other embodiments, three of A⁵, A⁶, A⁷, and A⁸ are CH or CR², and the remaining one of A⁵, A⁶, A⁷, and A⁸ is N or NR^{A2}, wherein R^{A2} is ═O. In certain embodiments, three of A⁵, A⁶, A⁷, and A⁸ are CH or CR², and the remaining one of A⁵, A⁶, A⁷, and A⁸ is N. In yet other embodiments, A⁵, A⁶, A⁷, and A⁸ are CH or CR².

In other embodiments, Ring A is

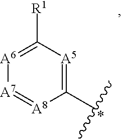

In some embodiments, Ring A is

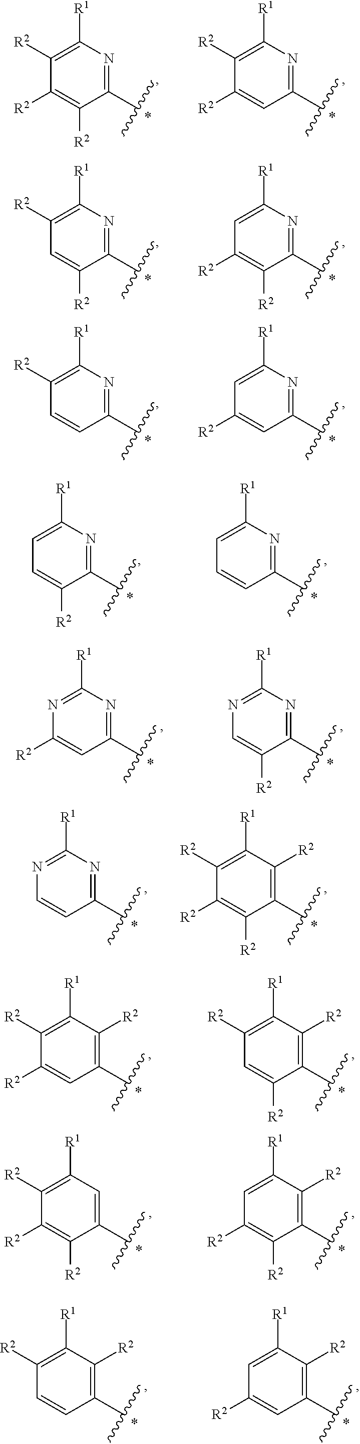

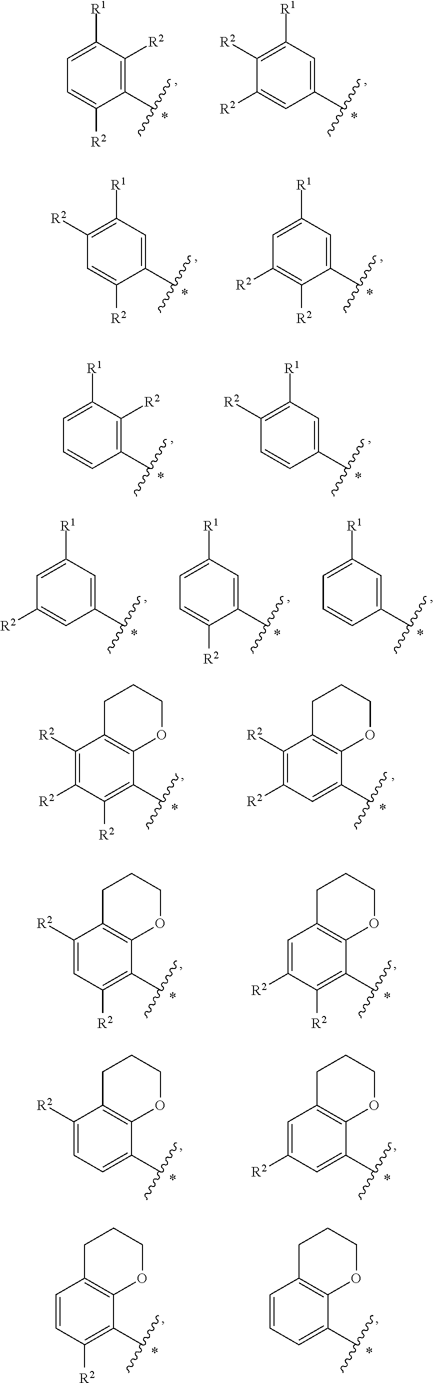

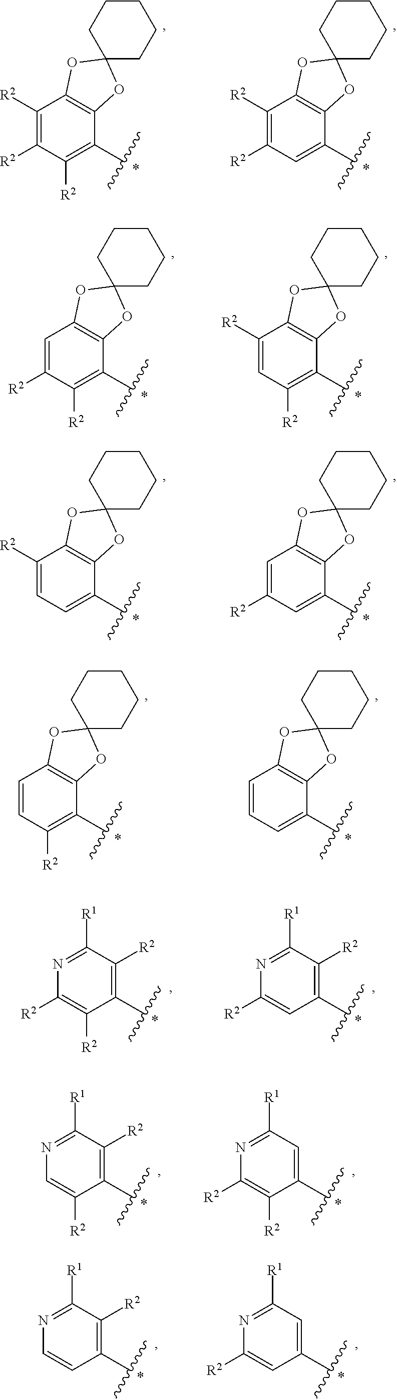

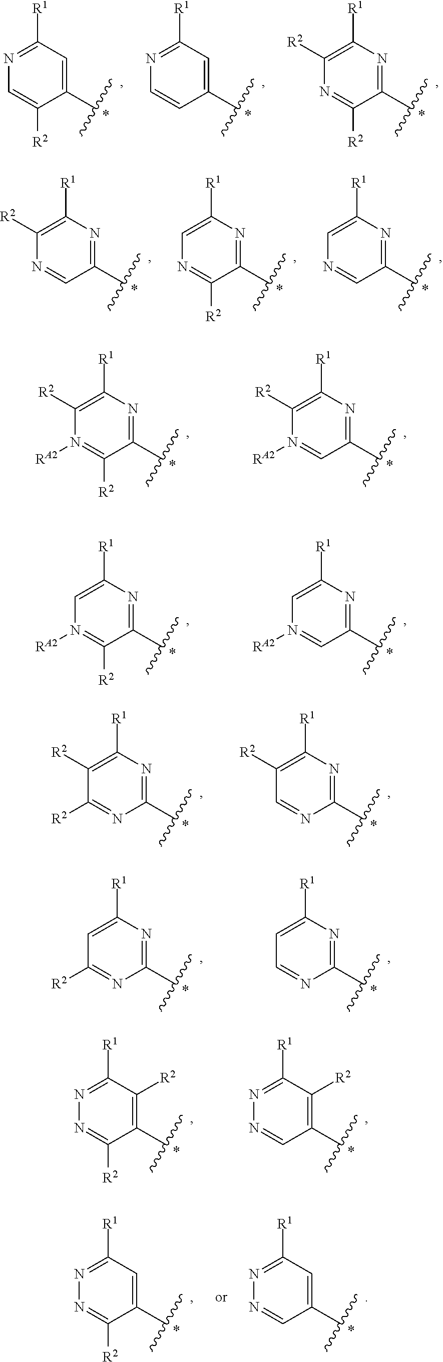

In other embodiments, Ring A is

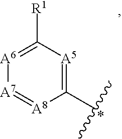

In some embodiments, Ring A is

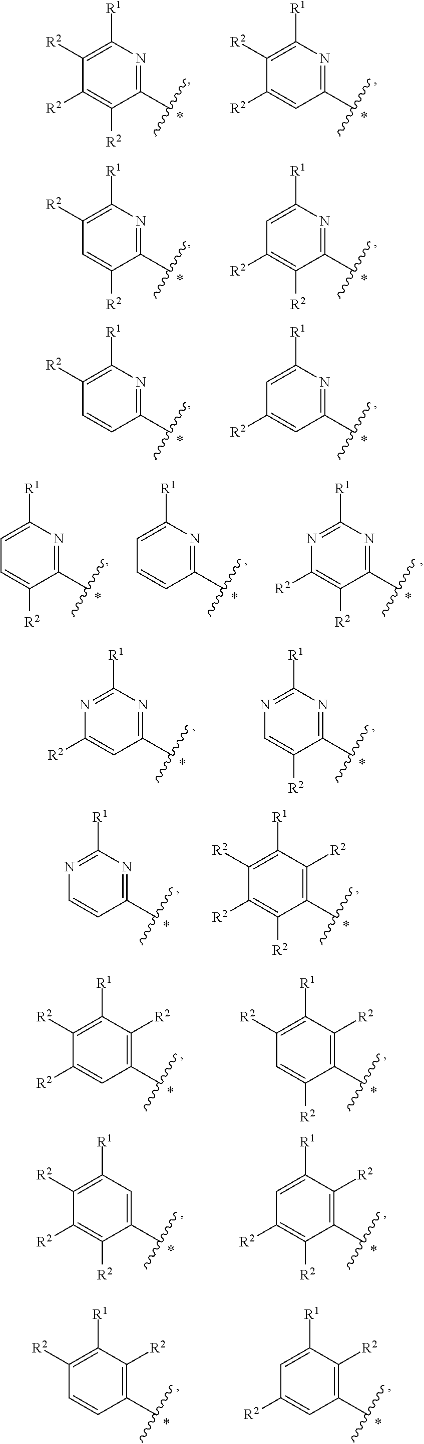

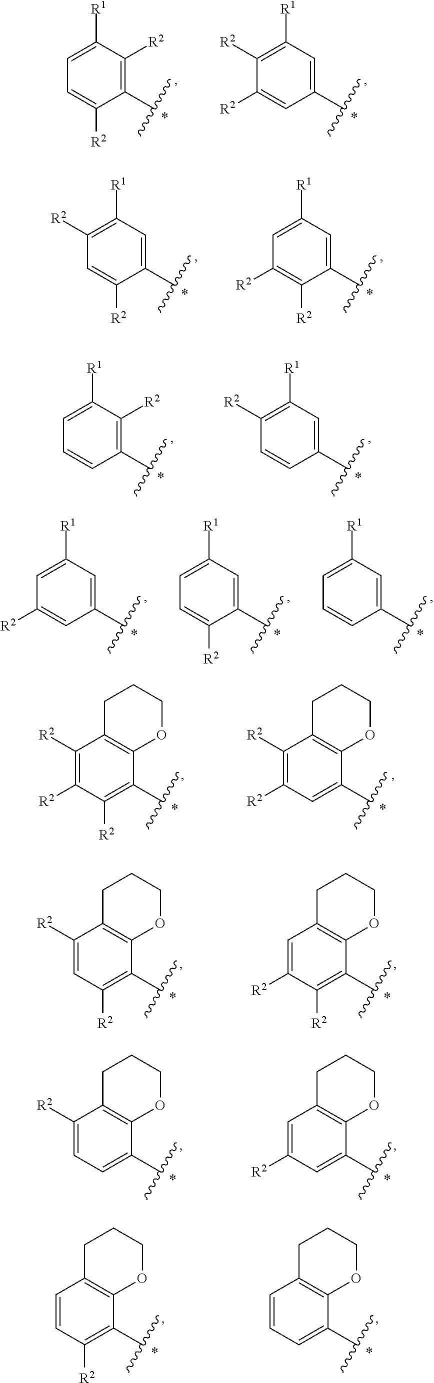

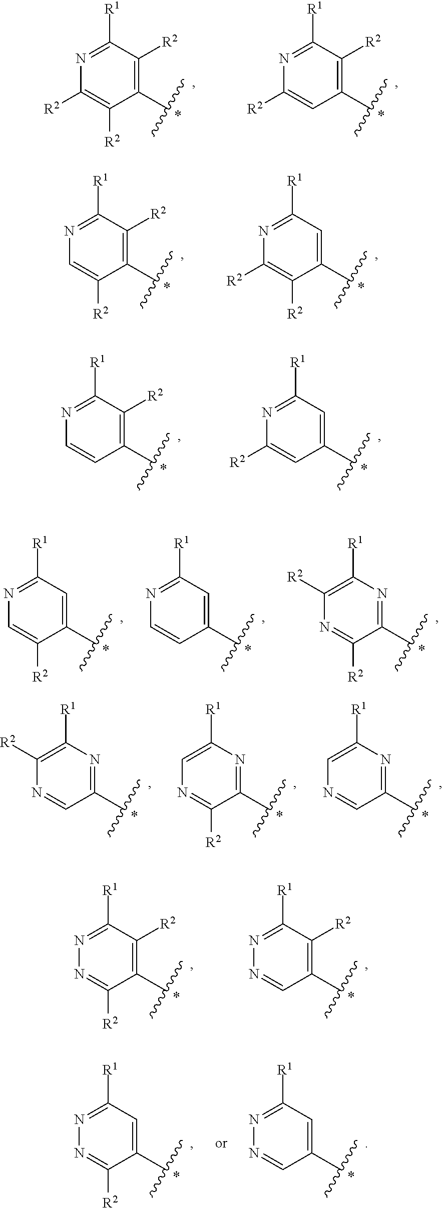

In some embodiments, Ring A is

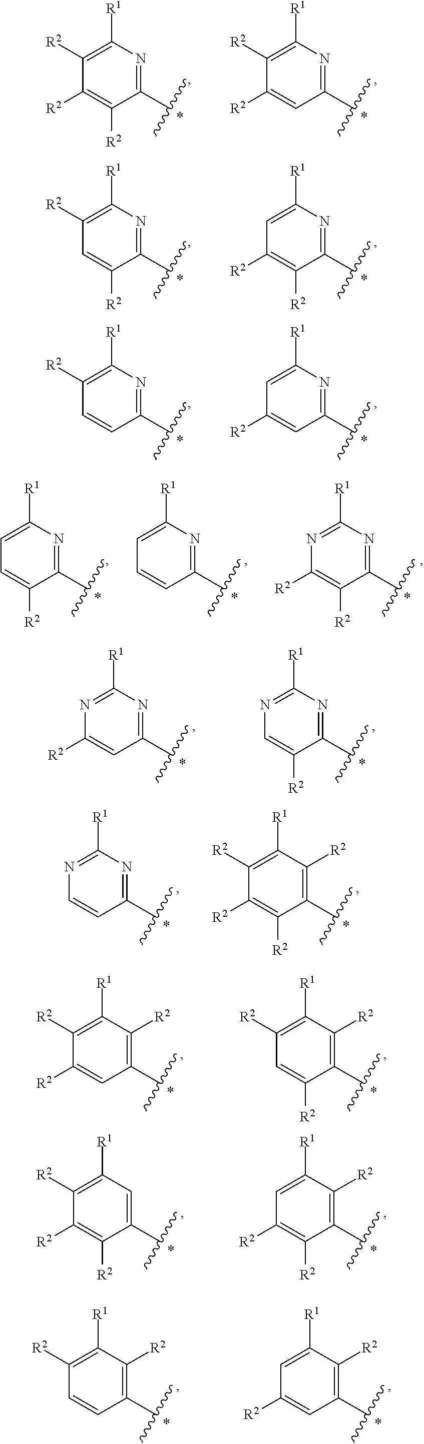

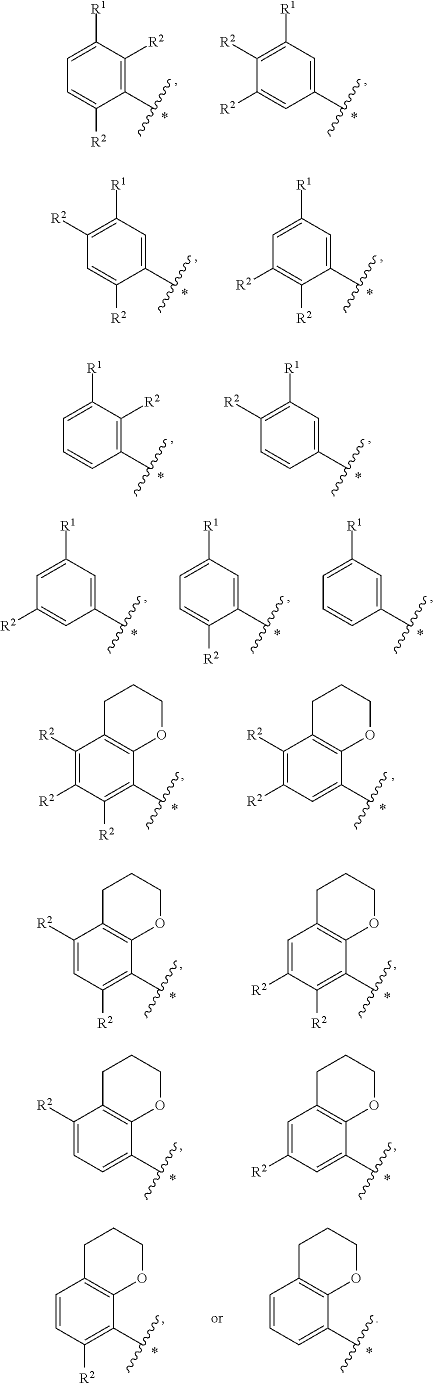

In certain embodiments, Ring A is

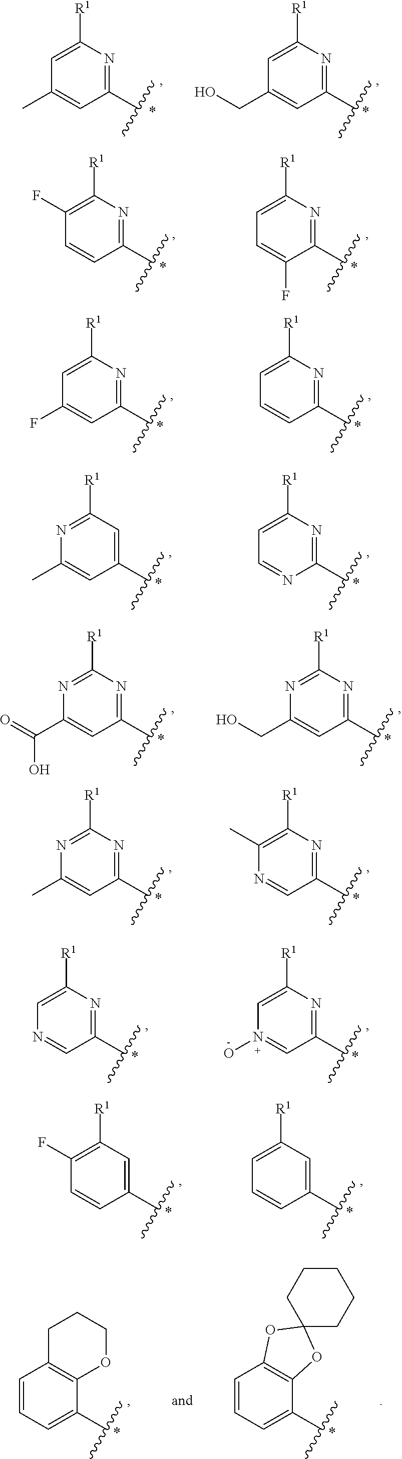

In certain embodiments, Ring A is

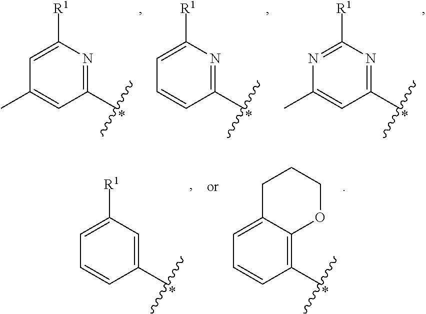

In some embodiments, R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20}, wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl. In some embodiments, cycloalkyl, cycloalkenyl, or heterocycloalkyl groups include spiro groups. In some embodiments, cycloalkyl, cycloalkenyl, or heterocycloalkyl groups include fused bicyclic groups. In some embodiments, cycloalkyl, cycloalkenyl, or heterocycloalkyl groups include bridged groups.

In some embodiments, R¹ is ₁₋₆ alkyl, C₃₋₆ cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂N^{a14}R^{a15}, —S(O)₂R^{a16}, or —(CR^{a17}R^{a18})₀₋₁C(O)NR^{a19}R^{a20}, wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R¹ is optionally substituted with one or more halogens. In some embodiments, cycloalkyl or heterocycloalkyl groups include spiro groups. In some embodiments, cycloalkyl or heterocycloalkyl groups include fused bicyclic groups.

In some embodiments, R¹ is C₁₋₆ alkyl, C₃₋₆ cycloalkyl, C₃₋₁₀ cycloalkenyl, 3- to 10-membered heterocycloalkyl, —NR^{a6}R^{a7}, —OR^{a10}, —S(O)₂NR^{a14}R^{a15}, or —S(O)₂R^{a16}, wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl. In some embodiments, R¹ is C₁₋₆ alkyl, C₃₋₆cycloalkyl, 3- to 10-membered heterocycloalkyl, —NR^{a6}R^{a7}, —S(O)₂NR^{a14}R^{a15}, or —S(O)₂R^{a16}, wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R¹ is optionally substituted with one or more halogens.

In some embodiments, R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl.

In other embodiments, R^{a14} and R^{a15} are taken together with the nitrogen to which they are attached to form a 3- to 10-membered heterocycloalkyl optionally substituted with one or more halo.

In some embodiments, R^{a6} and R^{a7} are each independently hydrogen, C₁₋₆ alkyl, C₃₋₆ cycloalkyl optionally substituted with one or more substituents independently selected from the group consisting of halo, or 5- to 12-membered heteroaryl optionally substituted with C₁₋₆ alkyl. In some embodiments, R^{a6} and R^{a7} are each independently hydrogen, C₁₋₆ alkyl, or 5- to 12-membered heteroaryl optionally substituted with C₁₋₆ alkyl. In some embodiments, R^{a6} and R^{a7} are each independently hydrogen, methyl, cyclobutyl optionally substituted with one or more fluoro, imidazolyl, methylimidazolyl, or pyrimidinyl. In some embodiments, R^{a6} and R^{a7} are each independently hydrogen, imidazolyl, methylimidazolyl, or pyrimidinyl. In some embodiments, —NR^{a6}R^{a7} is

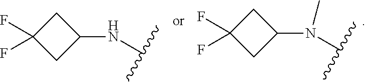

In some embodiments, R^{a10} is C₃₋₁₀ cycloalkyl. In some embodiments, —OR^{a10} is

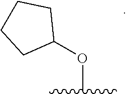 [SMILES: *OC1CCCC1]

In some embodiments, —S(O)₂NR^{a14}R^{a15} is

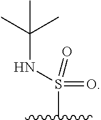 [SMILES: *S(=O)(=O)NC(C)(C)C]

In some embodiments, R^{a14} and R^{a15} are each independently hydrogen; C₁₋₆ alkyl optionally substituted with one, two, three, four, five, or more substituents independently selected from the group consisting of C₁₋₆ alkyl, C₂₋₆ alkenyl, C₃₋₁₀ cycloalkyl, —OH, —O(C₁₋₆ alkyl), —S(C₁₋₆ alkyl), and halo; C₂₋₆ alkenyl; C₃₋₁₀ cycloalkyl optionally substituted with one, two, three, four, five, or more substituents independently selected from the group consisting of C₂₋₆ alkenyl, C₃₋₁₀ cycloalkyl, halo, cyano, —OH, —O(C₁₋₆ alkyl), ═CR^{1a1}R^{1a2}, and C₁₋₆ alkyl optionally substituted with one, two, three, four, five, or more substituents independently selected from the group consisting of —OH, —O(C₁₋₆ alkyl), and halo, wherein R^{1a1} and R^{1a2} are each independently hydrogen or C₁₋₆ alkyl; C₃₋₁₀ cycloalkenyl; or 3- to 12-membered heterocycloalkyl optionally substituted with one, two, three, four, five, or more C₁₋₆ alkyl. In some embodiments, R^{a14} and R^{a15} are each independently hydrogen or C₁₋₆ alkyl. In some embodiments, R^{a14} is hydrogen and R^{a15} is butyl. In some embodiments, R^{a15} is tert-butyl. In some embodiments, —S(O)₂R^{a16} is

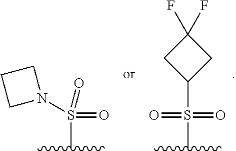 [SMILES: *S(=O)(=O)N1CCC1 ; *S(=O)(=O)C1CC(F)(F)C1]

In some embodiments, R^{a16} is C₃₋₁₀ cycloalkyl; or 3- to 12-membered heterocycloalkyl optionally substituted with one, two, three, four, five, or more substituents independently selected from the group consisting of C₁₋₆ alkyl or halo.

In some embodiments, R¹ is

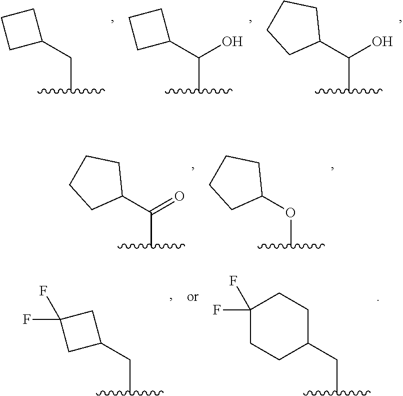

In some embodiments, R¹ is

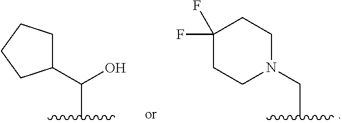 [SMILES: *C(O)C1CCCC1 ; *CN1CCC(F)(F)CC1]

In some embodiments, R¹ is C₁-C₆ alkyl optionally substituted with one or more substituents independently selected from the group consisting of halogen, —OH, oxo, cyano, C₃₋₁₀ cycloalkyl, and 3- to 10-membered heterocycloalkyl optionally substituted with one or more halo. In some embodiments, R¹ is C₁₋₆ alkyl optionally substituted with one, two, three, four, five, or more substituents independently selected from the group consisting of —OH, cyano, C₃₋₁₀ cycloalkyl, and 3- to 10-membered heterocycloalkyl optionally substituted with one, two, three, four, five, or more halo. In some embodiments, the 3- to 10-membered heterocycloalkyl is piperidinyl optionally substituted with one, two, three, four, five, or more halo. In other embodiments, the 3- to 10-membered heterocycloalkyl is pyrrolidinyl optionally substituted with one, two, three, four, five, or more halo. In other embodiments, the 3- to 10-membered heterocycloalkyl is azetidinyl optionally substituted with one, two, three, four, five, or more halo. In some embodiments, the 3- to 10-membered heterocycloalkyl is optionally substituted with one, two, three, four, five, or more fluoro. In some embodiments, the 3- to 10-membered heterocycloalkyl is piperidinyl optionally substituted with one, two, three, four, five, or more fluoro. In some embodiments, the 3- to 10-membered heterocycloalkyl is pyrrolidinyl optionally substituted with one, two, three, four, five, or more fluoro. In some embodiments, the 3- to 10-membered heterocycloalkyl is azetidinyl optionally substituted with one, two, three, four, five, or more fluoro.

In some embodiments, R¹ is

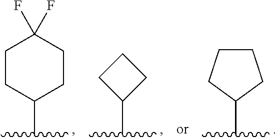

In some embodiments, R¹ is C₃₋₆ cycloalkyl optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl.

In some embodiments, R¹ is

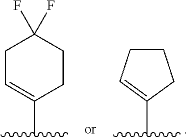 [SMILES: *C1=CCC(F)(F)CC1 ; *C1=CCCC1]

In some embodiments, R¹ is C₃₋₁₀ cycloalkenyl optionally substituted with one or more substituents independently selected from the group consisting of halogen.

In some embodiments, R¹ is

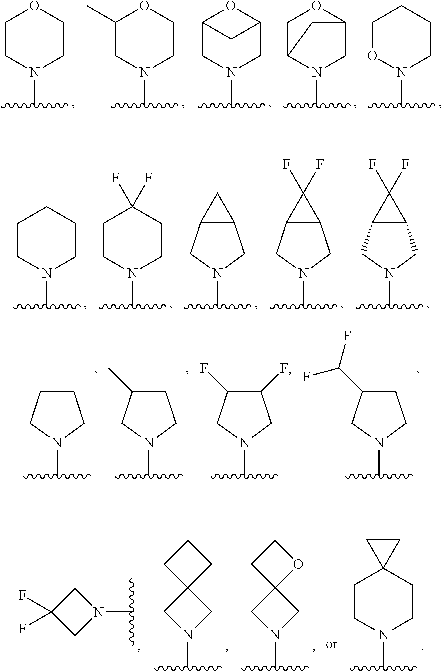

In some embodiments, R¹ is 3- to 10-membered heterocycloalkyl optionally substituted with one or more substituents independently selected from the group consisting of halogen, C₁₋₆ alkyl, and C₁₋₆ haloalkyl.

In some embodiments, R¹ is

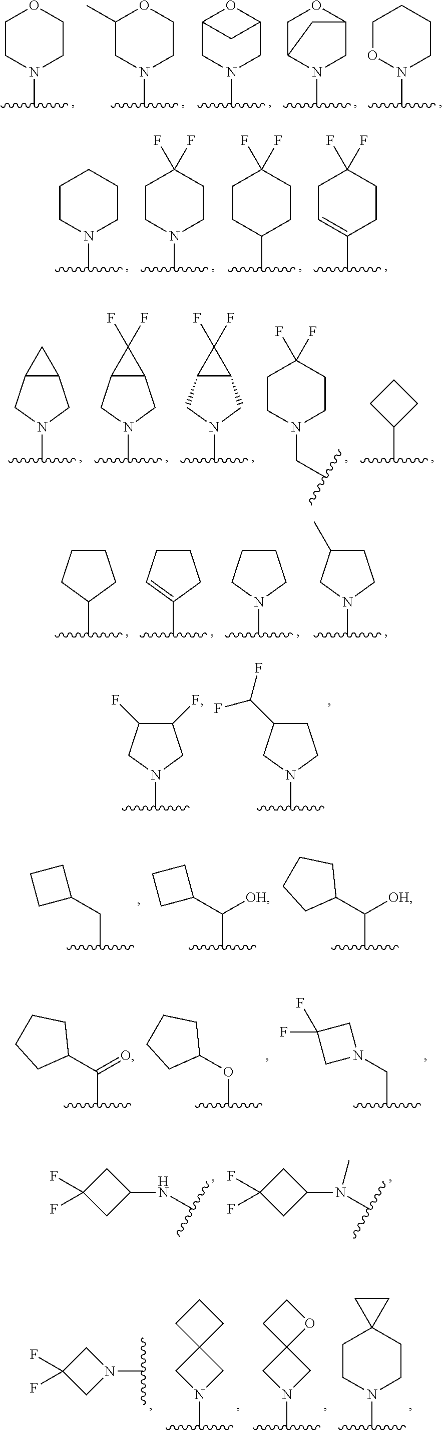

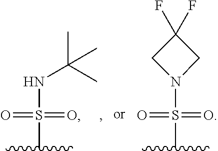

In some embodiments, R¹ is

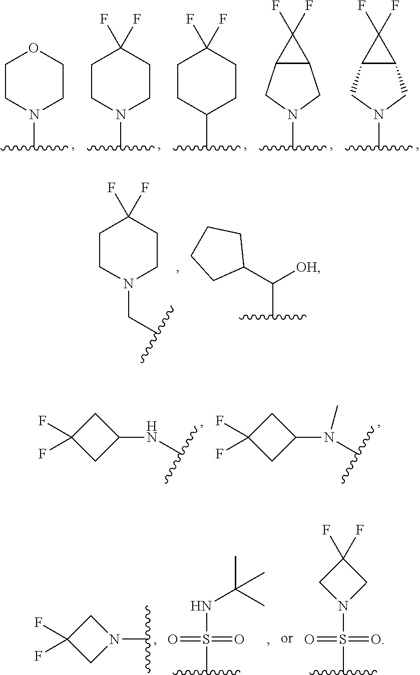

In some embodiments, each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring. In some embodiments, each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein R^{b1} and R^{b2} are independently optionally substituted with C₁-C₃ alkyl or R^{b1} and R^{b2} are taken together with the nitrogen to which they are attached to form a 3- to 6-membered ring. In some embodiments, each R² is independently halogen, C₁₋₃ alkyl, cyano, hydroxy, or NR^{b1}R^{b2}, wherein R^{b1} and R^{b2} are independently optionally substituted with C₁-C₃ alkyl. In some embodiments, each R² is independently C₁₋₃ alkyl, C₃₋₅ cycloalkyl, C₁₋₃ alkyloxy, C₃₋₅ cycloalkyloxy, or NR^{b1}R^{b2}, wherein R^{b1} and R^{b2} are taken together with the nitrogen to which they are attached to form a 3- to 6-membered ring. In some embodiments, each R² is halogen. In some embodiments, each R² is fluoro. In some embodiments, each R² is independently C₁₋₃ alkyl optionally substituted by one or more substituents selected from the group consisting of —OH and oxo. In other embodiments, each R² is independently C₁₋₃ alkyl. In certain embodiments, each R² is independently —CH₃. In some embodiments, R² is —CH₂OH. In other embodiments, each R² is —C(O)OH.

In still other embodiments, R¹ and the R² of A⁵ are taken together with the carbon atoms to which they are attached to form a C₃-C₆ cycloalkyl or a 3- to 6-membered heterocycloalkyl. In certain embodiments, Ring A is

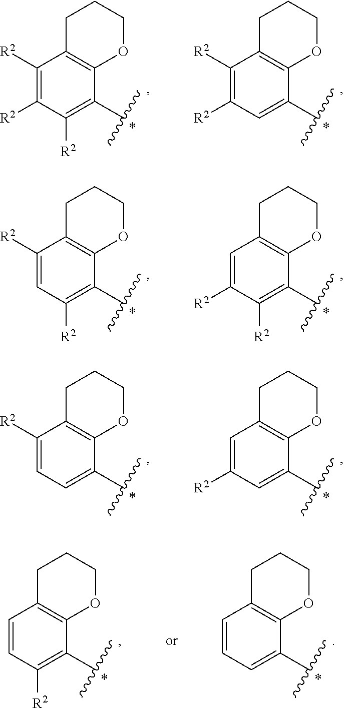

In certain other embodiments, Ring A is

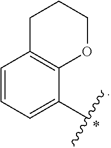 [SMILES: *c1cccc2c1OCCC2]

In still other embodiments, Ring A is

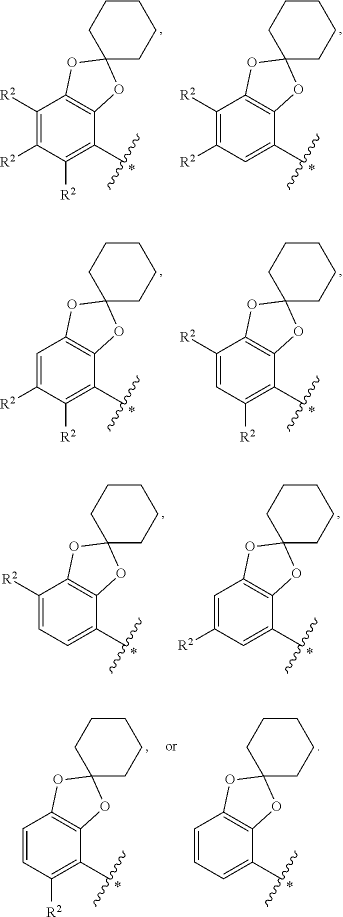

In certain other embodiments, Ring A is

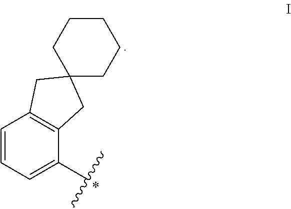 [SMILES: *c1cccc2c1CC1(CCCCC1)C2]

In some embodiments, B¹ and B² are each independently N, CH or CR^{B}, wherein R^{B} is halogen. In some embodiments, B¹ and B² are each independently N or CH.

In some embodiments, B¹ is N or CH. In some embodiments, B¹ is N. In other embodiments, B¹ is CH. In some embodiments, B¹ is CR^{B}, wherein R^{B} is halogen. In certain embodiments, B¹ is CR^{B}, wherein R^{B} is fluoro.

In some embodiments, B² is N or CH. In some embodiments, B² is N. In other embodiments, B² is CH. In some embodiments, B² is CR^{B}. wherein R^{B} is halogen. In certain embodiments, B² is CR^{B}, wherein R^{B} is fluoro.

In some embodiments, the ring

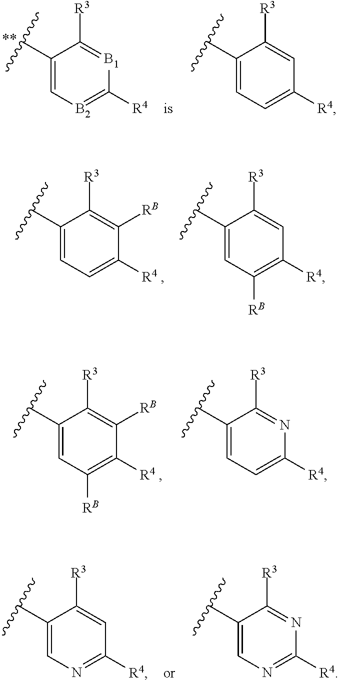

In some embodiments, the ring

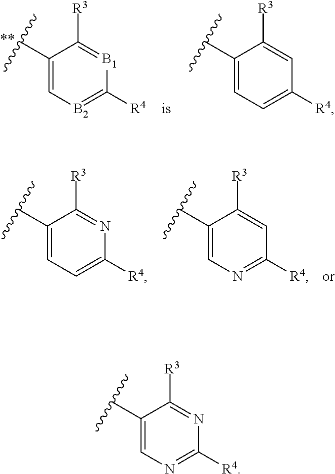

In certain embodiments, the ring

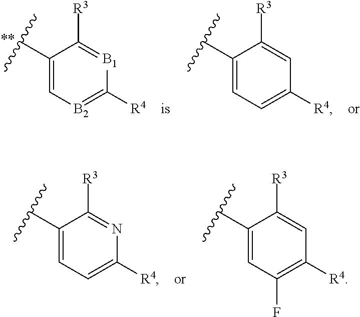

In certain embodiments, the ring

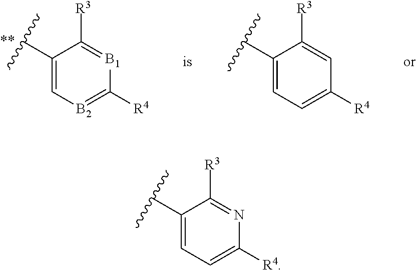

In some embodiments, the ring

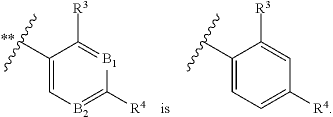

In some embodiments, the ring

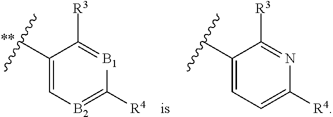

In some embodiments, the ring

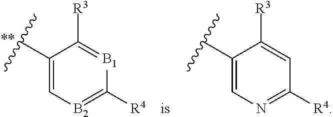

In some embodiments, the ring

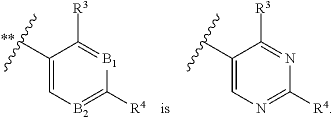

In some embodiments, the ring

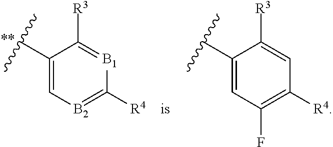

In some embodiments, R³ is piperidinyl, pyrrolidinyl, or azepanyl, wherein the piperidinyl, the pyrrolidinyl, or the azepanyl is optionally substituted with a C₃₋₁₀ cycloalkyl or 3-to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or azepanyl, or wherein the piperidinyl, pyrrolidinyl, or azepanyl are optionally substituted with a C₁₋₂ alkylene to form a bridged piperidinyl, pyrrolidinyl, or azepanyl ring system, wherein the piperidinyl, the pyrrolidinyl, the azepanyl, or the spirocyclic, fused, or bridged bicyclic ring system formed by the C₃₋₁₀ cycloalkyl, 3- to 10-membered heterocycloalkyl, or C₁₋₂ alkylene with piperidinyl, pyrrolidinyl, or azepanyl is optionally substituted with one or more substituents independently selected from the group consisting of C₁-C₃ alkyl, C₁-C₃ haloalkyl, and halo. In other embodiments, R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or the azepanyl, and wherein the piperidinyl, the pyrrolidinyl, the azepanyl or the spirocyclic or fused bicyclic ring system formed by the C₃₋₁₀ cycloalkyl or 3- to 10-membered heterocycloalkyl with piperidinyl, pyrrolidinyl, or the azepanyl is optionally substituted with one or more substituents independently selected from the group consisting of C₁-C₃ alkyl, C₁-C₃ haloalkyl, and halo.

In some embodiments, R³ is piperidinyl, wherein the piperidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, or wherein the piperidinyl is optionally substituted with a C₁₋₂ alkylene to form a bridged piperidinyl ring system, wherein the piperidinyl, or the spirocyclic, fused, or bridged bicyclic ring system formed by the C₃₋₁₀ cycloalkyl, 3- to 10-membered heterocycloalkyl, or C₁₋₂ alkylene with piperidinyl is optionally substituted with one or more substituents independently selected from the group consisting of C₁-C₃ alkyl, C₁-C₃ haloalkyl, and halo. In some embodiments, R³ is piperidinyl, wherein the piperidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, and wherein the piperidinyl or the spirocyclic or fused bicyclic ring system formed by the C₃₋₁₀ cycloalkyl or 3- to 10-membered heterocycloalkyl with piperidinyl is optionally substituted with one or more substituents independently selected from the group consisting of C₁-C₃ alkyl, C₁-C₃ haloalkyl, and halo. In some embodiments, R³ is piperidinyl, wherein the piperidinyl is optionally substituted with one or more substituents independently selected from the group consisting of C₁-C₃ alkyl, C₁-C₃ haloalkyl, and halo. In other embodiments, R³ is piperidinyl substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl and the spirocyclic or fused bicyclic ring system formed by the C₃₋₁₀ cycloalkyl or 3- to 10-membered heterocycloalkyl with piperidinyl is optionally substituted with one or more substituents independently selected from the group consisting of C₁-C₃ alkyl, C₁-C₃ haloalkyl, and halo. In some embodiments, R³ is

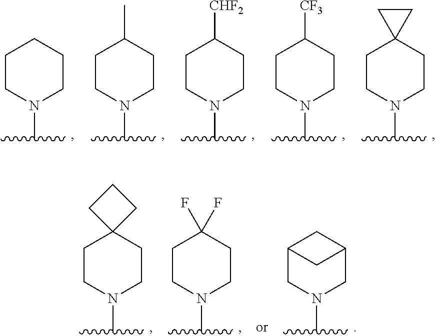

In some embodiments, R³ is

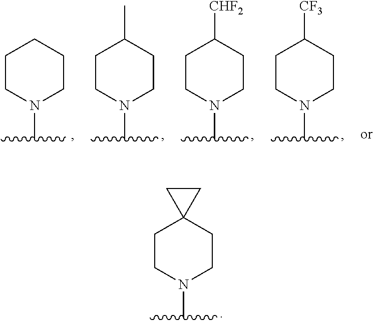

In some embodiments, R³ is

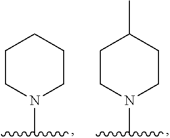

In certain embodiments, R³ is

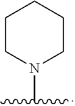 [SMILES: *N1CCCCC1]

In certain other embodiments, R³ is

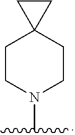 [SMILES: *N1CCC2(CC1)CC2]

In some embodiments, R³ is pyrrolidinyl, wherein the pyrrolidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the pyrrolidinyl, or wherein the pyrrolidinyl is optionally substituted with a C₁₋₂ alkylene to form a bridged pyrrolidinyl ring system, wherein the pyrrolidinyl, or the spirocyclic, fused, or bridged bicyclic ring system formed by the C₃₋₁₀ cycloalkyl, 3- to 10-membered heterocycloalkyl, or C₁₋₂ alkylene with the pyrrolidinyl is optionally substituted with one or more substituents independently selected from the group consisting of C₁-C₃ alkyl, C₁-C₃ haloalkyl, and halo. In other embodiments, R³ is pyrrolidinyl, wherein the pyrrolidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the pyrrolidinyl, and wherein the pyrrolidinyl or the spirocyclic or fused bicyclic ring system formed by the C₃₋₁₀ cycloalkyl or 3- to 10-membered heterocycloalkyl with pyrrolidinyl is optionally substituted with one or more substituents independently selected from the group consisting of C₁-C₃ alkyl, C₁-C₃ haloalkyl, and halo. In some embodiments, R³ is pyrrolidinyl, wherein the pyrrolidinyl is optionally substituted with one or more substituents independently selected from the group consisting of C₁-C₃ alkyl, C₁-C₃ haloalkyl, and halo. In some embodiments, R³ is

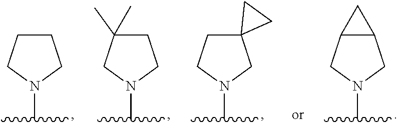

In some embodiments, R³ is

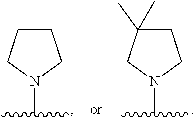

In certain embodiments, R³ is

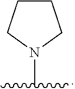 [SMILES: *N1CCCC1]

In certain other embodiments, R³ is

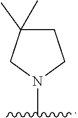 [SMILES: *N1CCC(C)(C)C1]

In other embodiments, R³ is pyrrolidinyl substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the pyrrolidinyl and the spirocyclic or fused bicyclic ring system formed by the C₃₋₁₀ cycloalkyl or 3- to 10-membered heterocycloalkyl with pyrrolidinyl is optionally substituted with one or more substituents independently selected from the group consisting of C₁-C₃ alkyl, C₁-C₃ haloalkyl, and halo.

In yet other embodiments, R³ is azepanyl, the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the azepanyl or wherein the azepanyl is optionally substituted with a C₁₋₂ alkylene to form a bridged azepanyl ring system, wherein the azepanyl or the spirocyclic, fused, or bridged bicyclic ring system formed by the C₃₋₁₀ cycloalkyl, 3- to 10-membered heterocycloalkyl, or C₁₋₂ alkylene with the azepanyl is optionally substituted with one or more substituents independently selected from the group consisting of C₁-C₃ alkyl, C₁-C₃ haloalkyl, and halo. In some embodiments, R³ is azepanyl, wherein the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the azepanyl, and wherein the azepanyl or the spirocyclic or fused bicyclic ring system formed by the C₃₋₁₀ cycloalkyl or 3- to 10-membered heterocycloalkyl with azepanyl is optionally substituted with one or more substituents independently selected from the group consisting of C₁-C₃ alkyl, C₁-C₃ haloalkyl, and halo. In some embodiments, R³ is azepanyl, wherein the azepanyl is optionally substituted with one or more substituents independently selected from the group consisting of C₁-C₃ alkyl, C₁-C₃ haloalkyl, and halo. In some embodiments, R³ is

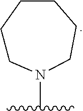 [SMILES: *N1CCCCCC1]

In other embodiments, R³ is azepanyl substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the azepanyl, and the spirocyclic or fused bicyclic ring system formed by the C₃₋₁₀ cycloalkyl or 3- to 10-membered heterocycloalkyl with azepanyl is optionally substituted with one or more substituents independently selected from the group consisting of C₁-C₃ alkyl, C₁-C₃ haloalkyl, and halo.

In some embodiments, R³ is

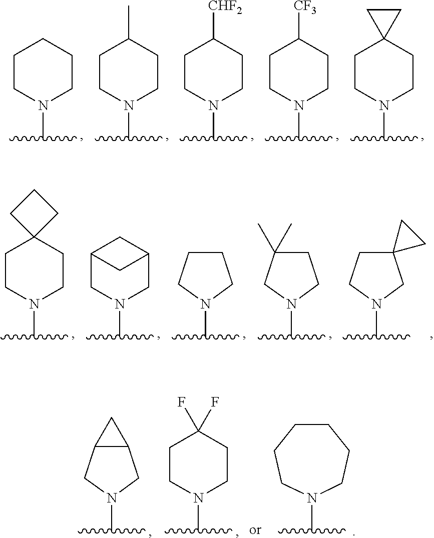

In some embodiments, R³ is

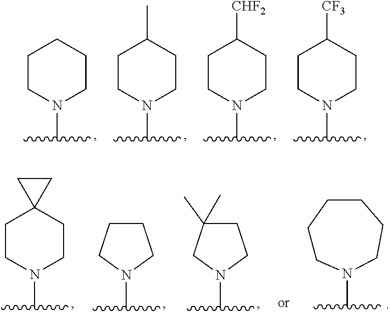

In one aspect, provided are compounds of Formula (III):

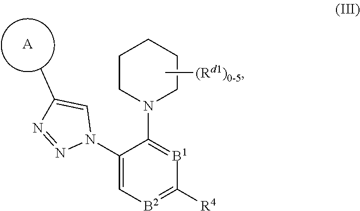

or a pharmaceutically acceptable salt thereof, wherein:
Ring A is

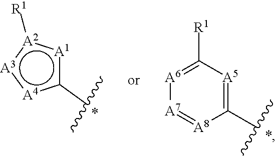

wherein
one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O;
wherein “*” indicates the point of attachment to V;
B¹ and B² are each independently N, CH or CR^{B}, wherein R^{B} is halogen;
R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 10-membered heterocycloalkyl;
wherein each R^{d1} is independently selected from the group consisting of C₁-C₃ alkyl, C₁-C₃ haloalkyl, and halo; or wherein two R^{d1} are taken together to form a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl; or wherein two R¹ are taken together to form a C₁₋₂ alkylene, wherein the C₁₋₂ alkylene forms a bridged piperidinyl ring system,
   wherein the spirocyclic, fused, or bridged bicyclic ring system formed by the C₃₋₁₀ cycloalkyl, 3- to 10-membered heterocycloalkyl, or C₁₋₂ alkylene with the piperidinyl is optionally substituted with one or more substituents independently selected from the group consisting of C₁-C₃ alkyl, C₁-C₃ haloalkyl, and halo;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH.

In some embodiments, R⁴ is hydrogen, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH.

In some embodiments, R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH. In some embodiments, R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

In some embodiments, R⁴ is hydrogen, halo, or —NR^{c5}S(O)₂R^{c6}. In some embodiments, R⁴ is hydrogen. In other embodiments, R⁴ is halo. In some embodiments, R⁴ is —NR^{c5}S(O)₂R^{c6}. In some embodiments, RCS is hydrogen or C₁₋₆ alkyl, wherein the C₁-C₆ alkyl is optionally substituted with one or more substituents independently selected from the group consisting of halo. In certain embodiments, R^{c5} is hydrogen. In some embodiments, R¹ is hydrogen. In some embodiments, R^{c6} is C₁₋₆ alkyl, wherein the C₁-C₆ alkyl is optionally substituted with one or more substituents independently selected from the group consisting of halo, —OH, and —C(O)—O—C₁-C₃ alkyl. In some embodiments, R⁶ is C₁₋₆ alkyl, wherein the C₁-C₆ alkyl is optionally substituted with one or more substituents independently selected from the group consisting of halo, —OH, and —C(O)—O—C₁-C₃ alkyl. In some embodiments, R⁶ is C₁₋₆ alkyl, wherein the C₁-C₆ alkyl is optionally substituted with one or more substituents independently selected from the group consisting of —OH and —C(O)—O—C₁-C₃ alkyl. In certain embodiments, R¹ is C₁₋₆ alkyl optionally substituted with one or more substituents independently selected from the group consisting of —OH and —C(O)—O—C₁-C₃ alkyl. In some embodiments, R⁶ is methyl or ethyl. In other embodiments, R⁶ is methyl substituted by —C(O)—O—C₁-C₃ alkyl. In some embodiments, R^{c6} is ethyl substituted by —OH or propyl substituted by —OH. In yet other embodiments, R¹ is ethyl substituted by —OH. In yet other embodiments, R^{c6} is C₃₋₁₀ cycloalkyl optionally substituted with one or more substituents independently selected from the group consisting of C₁-C₆ alkylene-OH. In some embodiments, R¹ is C₃₋₁₀ cycloalkyl. In certain embodiments, R^{c6} is cyclopropyl. In other embodiments, R^{c6} is C₃₋₁₀ cycloalkyl substituted with one or more substituents independently selected from the group consisting of C₁-C₆ alkylene-OH. In some embodiments, R^{c6} is cyclopropyl substituted with one or more substituents independently selected from the group consisting of —CH₂OH. In some embodiments, R⁴ is

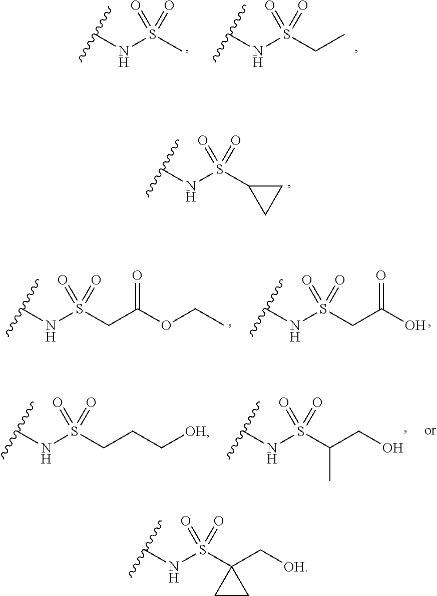

In some embodiments, NR^{c5}S(O)₂R^{c6} is

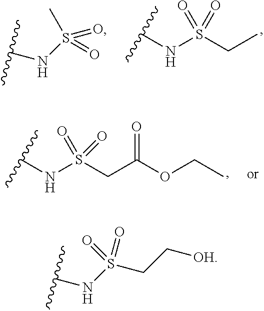

In some embodiments, R⁴ is H, Br,

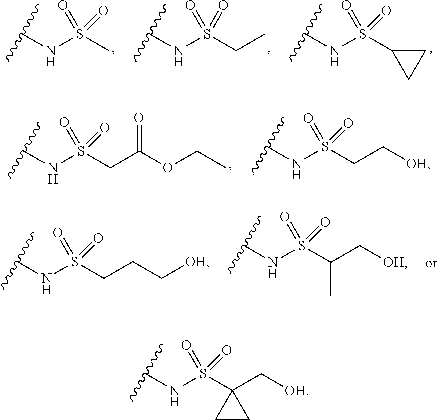

In some embodiments, R⁴ is H, Br,

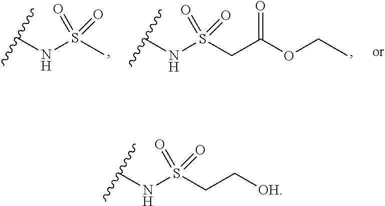

In certain embodiments, R⁴ is

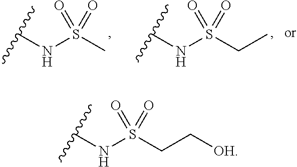

In certain embodiments, R⁴ is

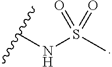 [SMILES: *NS(C)(=O)=O]

In some embodiments of the present aspect, the ring

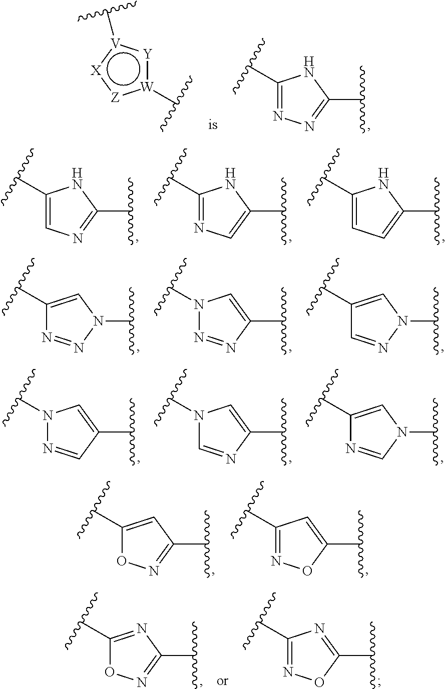

### Ring A is

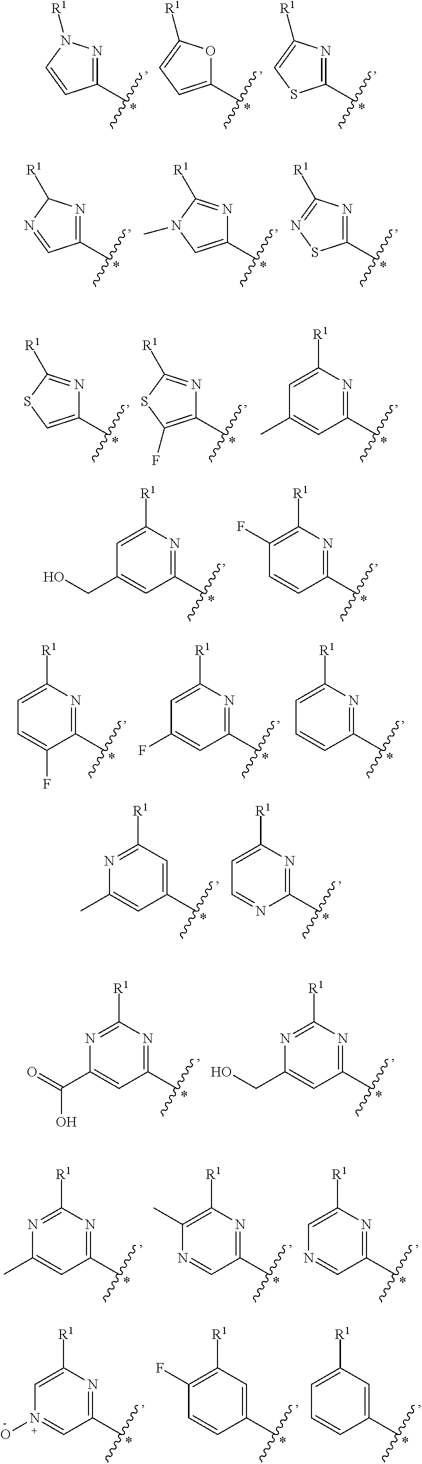

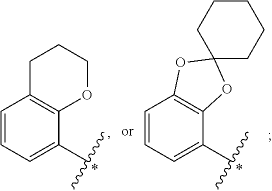 [SMILES: *c1cccc2c1OCCC2 ; "," ; "or" ; *c1cccc2c1OC1(CCCCC1)O2 ; ";"]

### R¹ is

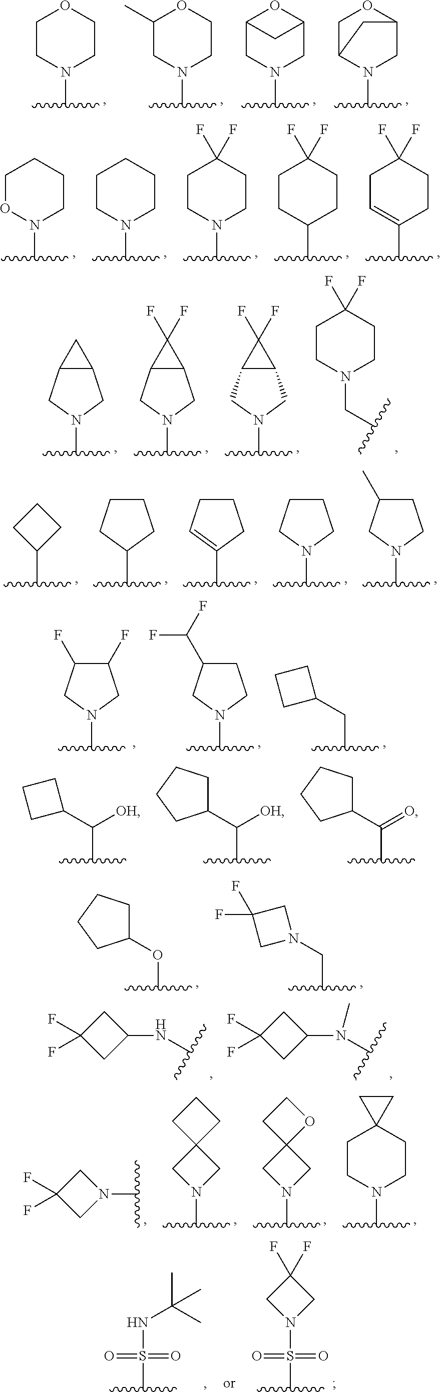

the ring

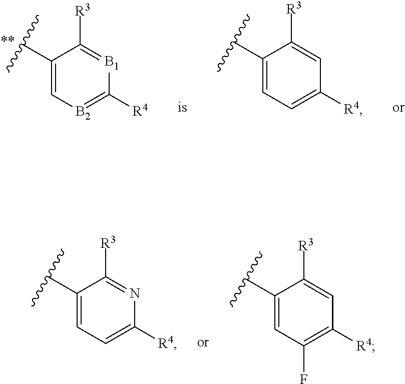

### R³ is

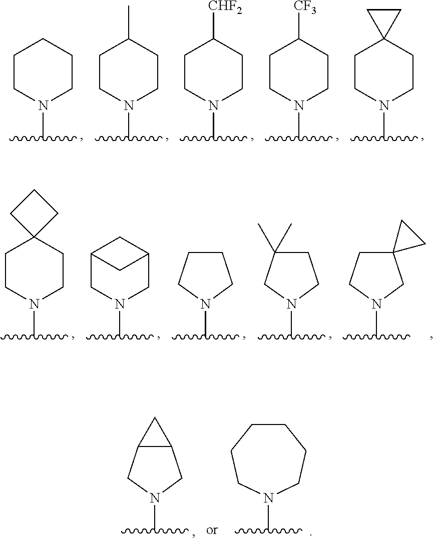

### and R⁴ is H, BR,

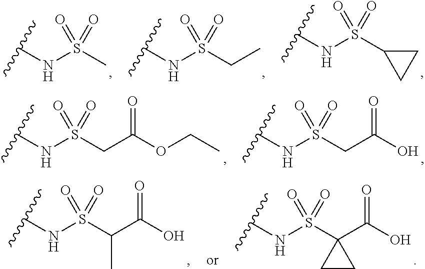

In some embodiments of the present aspect, the ring

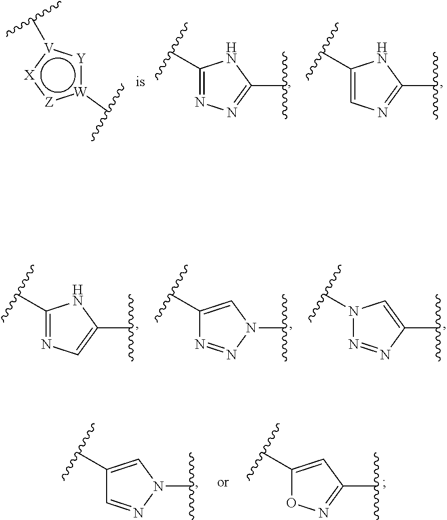

### Ring A is

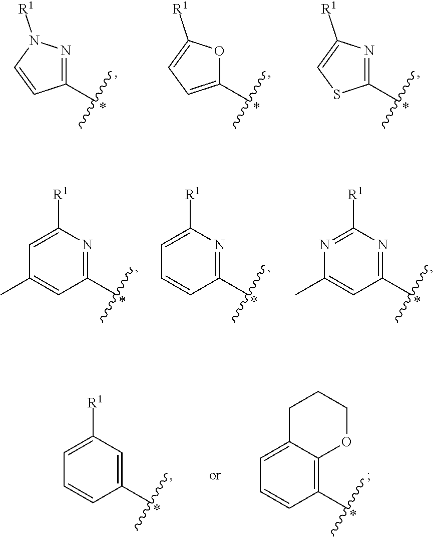

### R¹ is

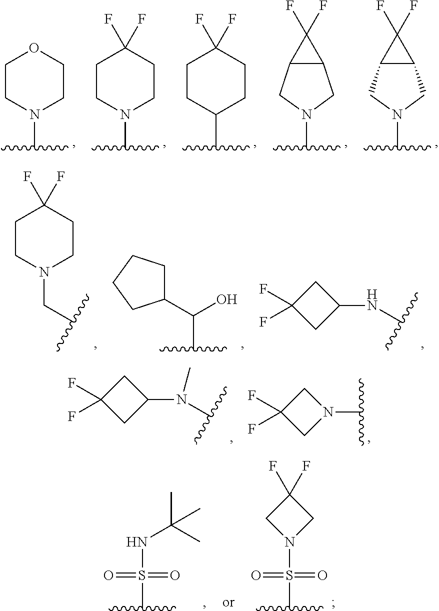

the ring

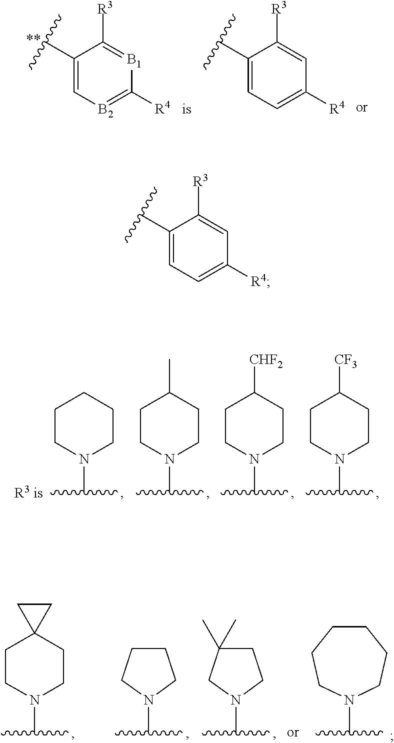

### and R⁴ is H, Br,

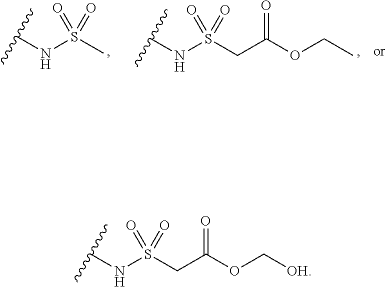

In some embodiments, provided herein are compounds and pharmaceutically acceptable salts thereof described in Table 1.

TABLE 1

| Compound | | |
|---|---|---|
| No. | Structure | Name |
| Compound 1 | 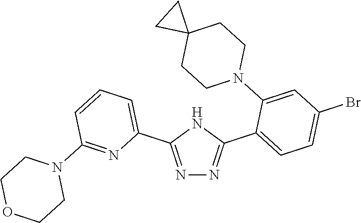 [SMILES: Brc1ccc(-c2nnc(-c3cccc(N4CCOCC4)n3)[nH]2)c(N2CCC3(CC2)CC3)c1] | 4-(6-(5-(4-bromo-2-(6-azaspiro[2.5]octan- 6-yl)phenyl)-4H-1,2,4-triazol-3- yl)pyridin-2-yl)morpholine |
| Compound 2 | 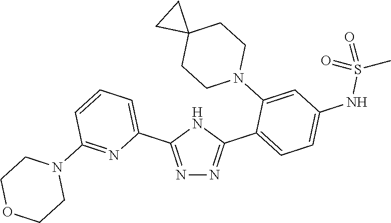 [SMILES: CS(=O)(=O)Nc1ccc(-c2nnc(-c3cccc(N4CCOCC4)n3)[nH]2)c(N2CCC3(CC2)CC3)c1] | N-(4-(5-(6-morpholinopyridin-2-yl)-4H- 1,2,4-triazol-3-yl)-3-(6-azaspiro[2.5]octan- 6-yl)phenyl)methanesulfonamide |
| Compound 3 | 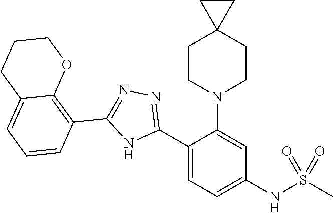 [SMILES: CS(=O)(=O)Nc1ccc(-c2nnc(-c3cccc4c3OCCC4)[nH]2)c(N2CCC3(CC2)CC3)c1] | N-(4-(5-(chroman-8-yl)-4H-1,2,4-triazol- 3-yl)-3-(6-azaspiro[2.5]octan-6- yl)phenyl)methanesulfonamide |
| Compound 4 | 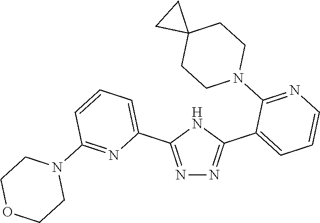 [SMILES: c1cc(-c2nnc(-c3cccnc3N3CCC4(CC3)CC4)[nH]2)nc(N2CCOCC2)c1] | 4-(6-(5-(2-(6-azaspiro[2.5]octan-6- yl)pyridin-3-yl)-4H-1,2,4-triazol-3- yl)pyridin-2-yl)morpholine |
| Compound 5 | 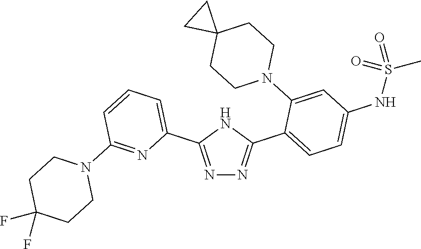 [SMILES: CS(=O)(=O)Nc1ccc(-c2nnc(-c3cccc(N4CCC(F)(F)CC4)n3)[nH]2)c(N2CCC3(CC2)CC3)c1] | N-(4-(5-(6-(4,4-difluoropiperidin-1- yl)pyridin-2-yl)-4H-1,2,4-triazol-3-yl)-3- (6-azaspiro[2.5]octan-6- yl)phenyl)methanesulfonamide |
| Compound 6 | 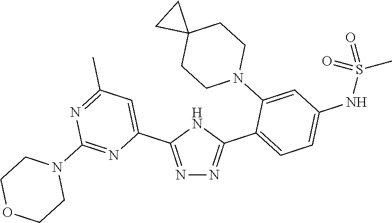 [SMILES: Cc1cc(-c2nnc(-c3ccc(NS(C)(=O)=O)cc3N3CCC4(CC3)CC4)[nH]2)nc(N2CCOCC2)n1] | N-(4-(5-(6-methyl-2- morpholinopyrimidin-4-yl)-4H-1,2,4- triazol-3-yl)-3-(6-azaspiro[2.5]octan-6- yl)phenyl)methanesulfonamide |
| Compound 7 | 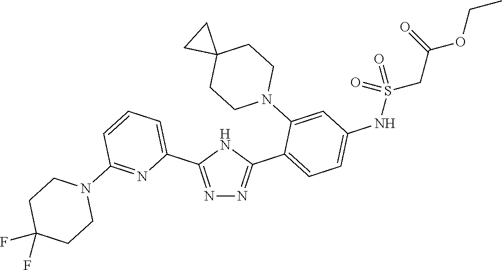 [SMILES: CCOC(=O)CS(=O)(=O)Nc1ccc(-c2nnc(-c3cccc(N4CCC(F)(F)CC4)n3)[nH]2)c(N2CCC3(CC2)CC3)c1] | ethyl 2-(N-(4-(5-(6-(4,4-difluoropiperidin- 1-yl)pyridin-2-yl)-4H-1,2,4-triazol-3-yl)- 3-(6-azaspiro[2.5]octan-6- yl)phenyl)sulfamoyl)acetate |
| Compound 8 | 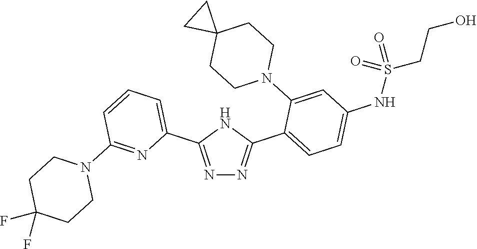 [SMILES: O=S(=O)(CCO)Nc1ccc(-c2nnc(-c3cccc(N4CCC(F)(F)CC4)n3)[nH]2)c(N2CCC3(CC2)CC3)c1] | N-(4-(5-(6-(4,4-difluoropiperidin-1- yl)pyridin-2-yl)-4H-1,2,4-triazol-3-yl)-3- (6-azaspiro[2.5]octan-6-yl)phenyl)-2- hydroxyethane-1-sulfonamide |
| Compound 9 | 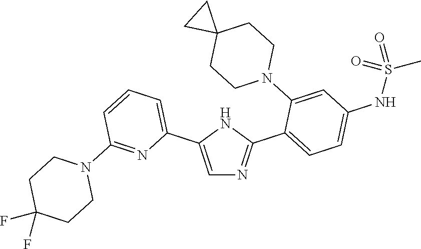 [SMILES: CS(=O)(=O)Nc1ccc(-c2ncc(-c3cccc(N4CCC(F)(F)CC4)n3)[nH]2)c(N2CCC3(CC2)CC3)c1] | N-(4-(5-(6-(4,4-difluoropiperidin-1- yl)pyridin-2-yl)-1H-imidazol-2-yl)-3-(6- azaspiro[2.5]octan-6- yl)phenyl)methanesulfonamide |
| Compound 10 | 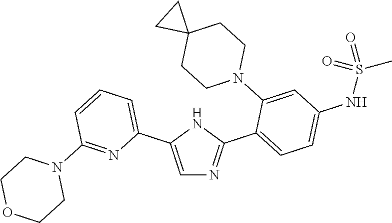 [SMILES: CS(=O)(=O)Nc1ccc(-c2ncc(-c3cccc(N4CCOCC4)n3)[nH]2)c(N2CCC3(CC2)CC3)c1] | N-(4-(5-(6-morpholinopyridin-2-yl)-1H- imidazol-2-yl)-3-(6-azaspiro[2.5]octan-6- yl)phenyl)methanesulfonamide |
| Compound 11 | 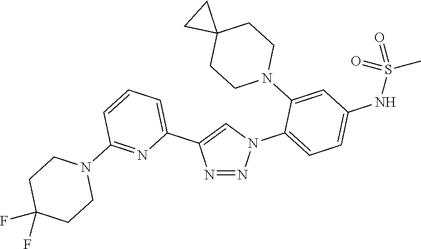 [SMILES: CS(=O)(=O)Nc1ccc(-n2cc(-c3cccc(N4CCC(F)(F)CC4)n3)nn2)c(N2CCC3(CC2)CC3)c1] | N-(4-(4-(6-(4,4-difluoropiperidin-1- yl)pyridin-2-yl)-1H-1,2,3-triazol-1-yl)-3- (6-azaspiro[2.5]octan-6- yl)phenyl)methanesulfonamide |
| Compound 12 | 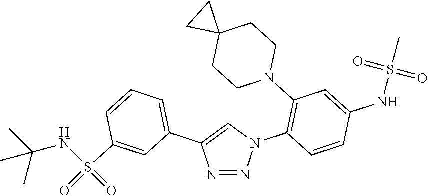 [SMILES: CC(C)(C)NS(=O)(=O)c1cccc(-c2cn(-c3ccc(NS(C)(=O)=O)cc3N3CCC4(CC3)CC4)nn2)c1] | N-(tert-butyl)-3-(1-(4- (methylsulfonamido)-2-(6- azaspiro[2.5]octan-6-yl)phenyl)-1H-1,2,3- triazol-4-yl)benzenesulfonamide |
| Compound 13 | 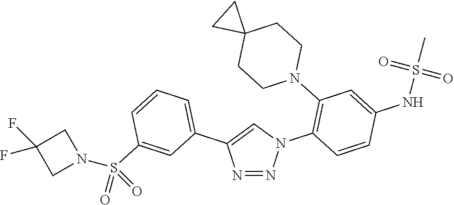 [SMILES: CS(=O)(=O)Nc1ccc(-n2cc(-c3cccc(S(=O)(=O)N4CC(F)(F)C4)c3)nn2)c(N2CCC3(CC2)CC3)c1] | N-(4-(4-(3-((3,3-difluoroazetidin-1- yl)sulfonyl)phenyl)-1H-1,2,3-triazol-1-yl)- 3-(6-azaspiro[2.5]octan-6- yl)phenyl)methanesulfonamide |
| Compound 14 | 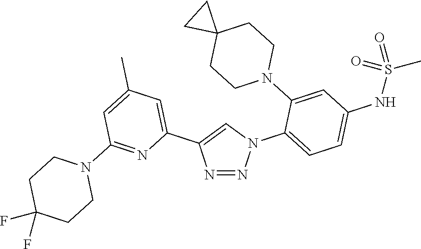 [SMILES: Cc1cc(-c2cn(-c3ccc(NS(C)(=O)=O)cc3N3CCC4(CC3)CC4)nn2)nc(N2CCC(F)(F)CC2)c1] | N-(4-(4-(6-(4,4-difluoropiperidin-1-yl)-4- methylpyridin-2-yl)-1H-1,2,3-triazol-1- yl)-3-(6-azaspiro[2.5]octan-6- yl)phenyl)methanesulfonamide |
| Compound 15 | 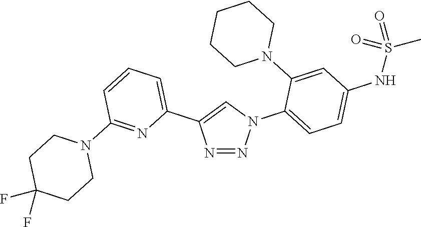 [SMILES: CS(=O)(=O)Nc1ccc(-n2cc(-c3cccc(N4CCC(F)(F)CC4)n3)nn2)c(N2CCCCC2)c1] | N-(4-(4-(6-(4,4-difluoropiperidin-1- yl)pyridin-2-yl)-1H-1,2,3-triazol-1-yl)-3- (piperidin-1- yl)phenyl)methanesulfonamide |
| Compound 16 | 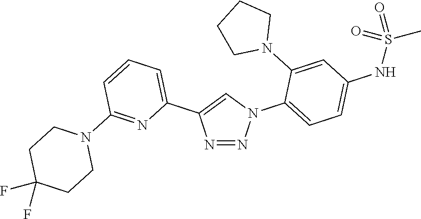 [SMILES: CS(=O)(=O)Nc1ccc(-n2cc(-c3cccc(N4CCC(F)(F)CC4)n3)nn2)c(N2CCCC2)c1] | N-(4-(4-(6-(4,4-difluoropiperidin-1- yl)pyridin-2-yl)-1H-1,2,3-triazol-1-yl)-3- (pyrrolidin-1- yl)phenyl)methanesulfonamide |
| Compound 17 | 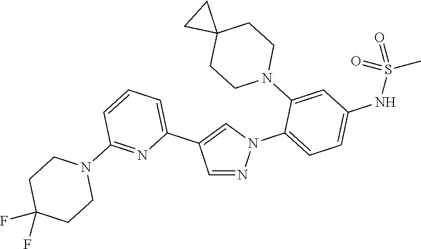 [SMILES: CS(=O)(=O)Nc1ccc(-n2cc(-c3cccc(N4CCC(F)(F)CC4)n3)cn2)c(N2CCC3(CC2)CC3)c1] | N-(4-(4-(6-(4,4-difluoropiperidin-1- yl)pyridin-2-yl)-1H-pyrazol-1-yl)-3-(6- azaspiro[2.5]octan-6- yl)phenyl)methanesulfonamide |
| Compound 18 | 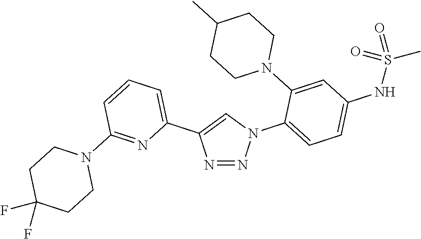 [SMILES: CC1CCN(c2cc(NS(C)(=O)=O)ccc2-n2cc(-c3cccc(N4CCC(F)(F)CC4)n3)nn2)CC1] | N-(4-(4-(6-(4,4-difluoropiperidin-1- yl)pyridin-2-yl)-1H-1,2,3-triazol-1-yl)-3- (4-methylpiperidin-1- yl)phenyl)methanesulfonamide |
| Compound 19 | 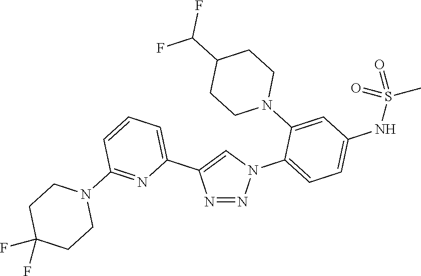 [SMILES: CS(=O)(=O)Nc1ccc(-n2cc(-c3cccc(N4CCC(F)(F)CC4)n3)nn2)c(N2CCC(C(F)F)CC2)c1] | N-(3-(4-(difluoromethyl)piperidin-1-yl)-4- (4-(6-(4,4-difluoropiperidin-1-yl)pyridin- 2-yl)-1H-1,2,3-triazol-1- yl)phenyl)methanesulfonamide |
| Compound 20 | 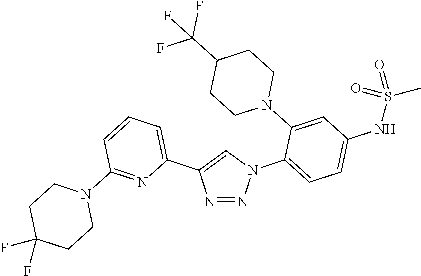 [SMILES: CS(=O)(=O)Nc1ccc(-n2cc(-c3cccc(N4CCC(F)(F)CC4)n3)nn2)c(N2CCC(C(F)(F)F)CC2)c1] | N-(4-(4-(6-(4,4-difluoropiperidin-1- yl)pyridin-2-yl)-1H-1,2,3-triazol-1-yl)-3- (4-(trifluoromethyl)piperidin-1- yl)phenyl)methanesulfonamide |
| Compound 21 | 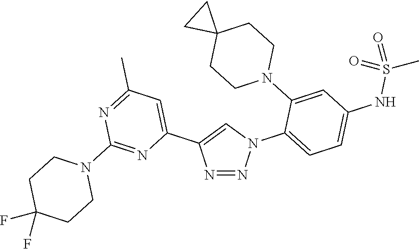 [SMILES: Cc1cc(-c2cn(-c3ccc(NS(C)(=O)=O)cc3N3CCC4(CC3)CC4)nn2)nc(N2CCC(F)(F)CC2)n1] | N-(4-(4-(2-(4,4-difluoropiperidin-1-yl)-6- methylpyrimidin-4-yl)-1H-1,2,3-triazol-1- yl)-3-(6-azaspiro[2.5]octan-6- yl)phenyl)methanesulfonamide |
| Compound 22 | 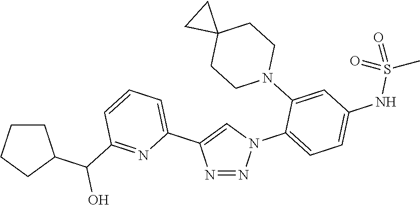 [SMILES: CS(=O)(=O)Nc1ccc(-n2cc(-c3cccc(C(O)C4CCCC4)n3)nn2)c(N2CCC3(CC2)CC3)c1] | N-(4-(4-(6- (cyclopentyl(hydroxy)methyl)pyridin-2- yl)-1H-1,2,3-triazol-1-yl)-3-(6- azaspiro[2.5]octan-6- yl)phenyl)methanesulfonamide |
| Compound 23 | 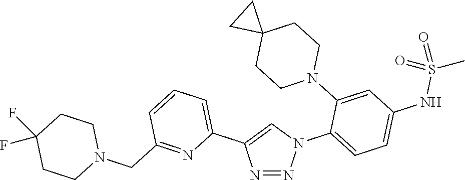 [SMILES: CS(=O)(=O)Nc1ccc(-n2cc(-c3cccc(CN4CCC(F)(F)CC4)n3)nn2)c(N2CCC3(CC2)CC3)c1] | N-(4-(4-(6-((4,4-difluoropiperidin-1- yl)methyl)pyridin-2-yl)-1H-1,2,3-triazol- 1-yl)-3-(6-azaspiro[2.5]octan-6- yl)phenyl)methanesulfonamide |
| Compound 25 | 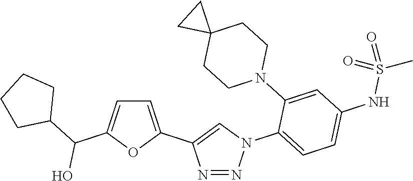 [SMILES: CS(=O)(=O)Nc1ccc(-n2cc(-c3ccc(C(O)C4CCCC4)o3)nn2)c(N2CCC3(CC2)CC3)c1] | N-(4-(4-(5- (cyclopentyl(hydroxy)methyl)furan-2-yl)- 1H-1,2,3-triazol-1-yl)-3-(6- azaspiro[2.5]octan-6- yl)phenyl)methanesulfonamide |
| Compound 26 | 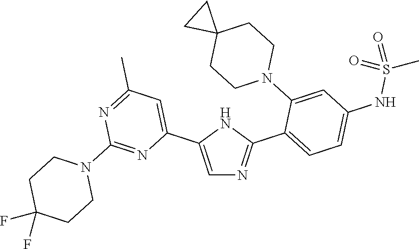 [SMILES: Cc1cc(-c2cnc(-c3ccc(NS(C)(=O)=O)cc3N3CCC4(CC3)CC4)[nH]2)nc(N2CCC(F)(F)CC2)n1] | N-(4-(5-(2-(4,4-difluoropiperidin-1-yl)-6- methylpyrimidin-4-yl)-1H-imidazol-2-yl)- 3-(6-azaspiro[2.5]octan-6- yl)phenyl)methanesulfonamide |
| Compound 27 | 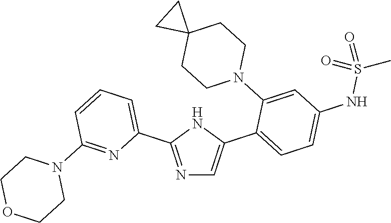 [SMILES: CS(=O)(=O)Nc1ccc(-c2cnc(-c3cccc(N4CCOCC4)n3)[nH]2)c(N2CCC3(CC2)CC3)c1] | N-(4-(2-(6-morpholinopyridin-2-yl)-1H- imidazol-5-yl)-3-(6-azaspiro[2.5]octan-6- yl)phenyl)methanesulfonamide |
| Compound 28 | 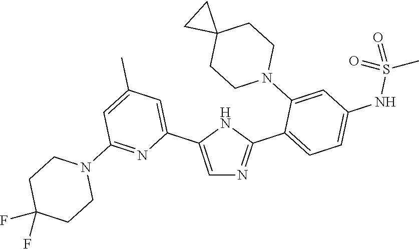 [SMILES: Cc1cc(-c2cnc(-c3ccc(NS(C)(=O)=O)cc3N3CCC4(CC3)CC4)[nH]2)nc(N2CCC(F)(F)CC2)c1] | N-(4-(5-(6-(4,4-difluoropiperidin-1-yl)-4- methylpyridin-2-yl)-1H-imidazol-2-yl)-3- (6-azaspiro[2.5]octan-6- yl)phenyl)methanesulfonamide |
| Compound 29 | 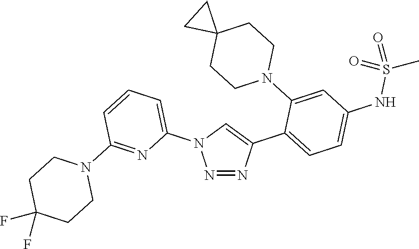 [SMILES: CS(=O)(=O)Nc1ccc(-c2cn(-c3cccc(N4CCC(F)(F)CC4)n3)nn2)c(N2CCC3(CC2)CC3)c1] | N-(4-(1-(6-(4,4-difluoropiperidin-1- yl)pyridin-2-yl)-1H-1,2,3-triazol-4-yl)-3- (6-azaspiro[2.5]octan-6- yl)phenyl)methanesulfonamide |
| Compound 30 | 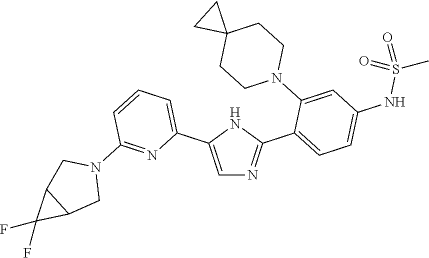 [SMILES: CS(=O)(=O)Nc1ccc(-c2ncc(-c3cccc(N4CC5C(C4)C5(F)F)n3)[nH]2)c(N2CCC3(CC2)CC3)c1] | N-(4-(5-(6-(6,6-difluoro-3- azabicyclo[3.1.0]hexan-3-yl)pyridin-2-yl)- 1H-imidazol-2-yl)-3-(6- azaspiro[2.5]octan-6- yl)phenyl)methanesulfonamide |
| Compound 30-1 | 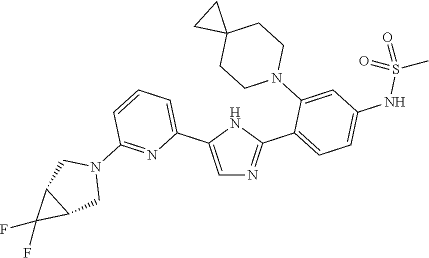 [SMILES: CS(=O)(=O)Nc1ccc(-c2ncc(-c3cccc(N4C[C@@H]5[C@H](C4)C5(F)F)n3)[nH]2)c(N2CCC3(CC2)CC3)c1] | N-(4-(5-(6-((1R,5S)-6,6-difluoro-3- azabicyclo[3.1.0]hexan-3-yl)pyridin-2-yl)- 1H-imidazol-2-yl)-3-(6- azaspiro[2.5]octan-6- yl)phenyl)methanesulfonamide |
| Compound 31 | 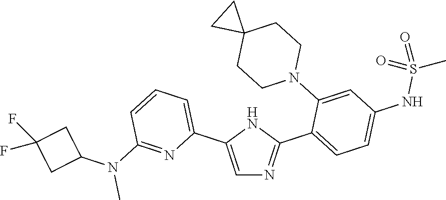 [SMILES: CN(c1cccc(-c2cnc(-c3ccc(NS(C)(=O)=O)cc3N3CCC4(CC3)CC4)[nH]2)n1)C1CC(F)(F)C1] | N-(4-(5-(6-((3,3- difluorocyclobutyl)(methyl)amino)pyridin- 2-yl)-1H-imidazol-2-yl)-3-(6- azaspiro[2.5]octan-6- yl)phenyl)methanesulfonamide |
| Compound 32 | 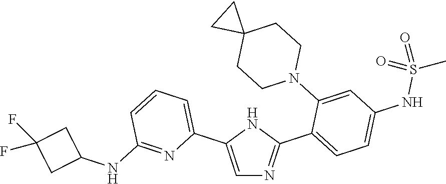 [SMILES: CS(=O)(=O)Nc1ccc(-c2ncc(-c3cccc(NC4CC(F)(F)C4)n3)[nH]2)c(N2CCC3(CC2)CC3)c1] | N-(4-(5-(6-((3,3- difluorocyclobutyl)amino)pyridin-2-yl)- 1H-imidazol-2-yl)-3-(6- azaspiro[2.5]octan-6- yl)phenyl)methanesulfonamide |
| Compound 33 | 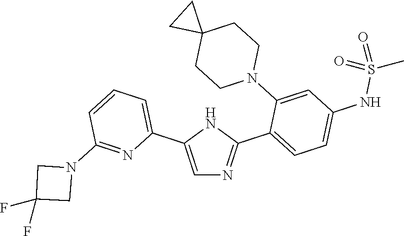 [SMILES: CS(=O)(=O)Nc1ccc(-c2ncc(-c3cccc(N4CC(F)(F)C4)n3)[nH]2)c(N2CCC3(CC2)CC3)c1] | N-(4-(5-(6-(3,3-difluoroazetidin-1- yl)pyridin-2-yl)-1H-imidazol-2-yl)-3-(6- azaspiro[2.5]octan-6- yl)phenyl)methanesulfonamide |
| Compound 34 | 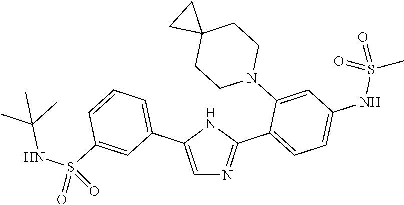 [SMILES: CC(C)(C)NS(=O)(=O)c1cccc(-c2cnc(-c3ccc(NS(C)(=O)=O)cc3N3CCC4(CC3)CC4)[nH]2)c1] | N-(tert-butyl)-3-(2-(4- (methylsulfonamido)-2-(6- azaspiro[2.5]octan-6-yl)phenyl)-1H- imidazol-5-yl)benzenesulfonamide |
| Compound 35 | 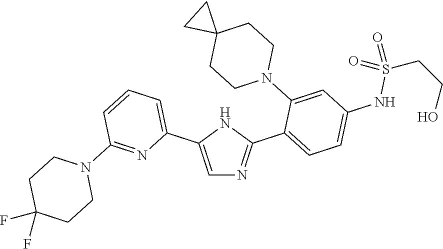 [SMILES: O=S(=O)(CCO)Nc1ccc(-c2ncc(-c3cccc(N4CCC(F)(F)CC4)n3)[nH]2)c(N2CCC3(CC2)CC3)c1] | N-(4-(5-(6-(4,4-difluoropiperidin-1- yl)pyridin-2-yl)-1H-imidazol-2-yl)-3-(6- azaspiro[2.5]octan-6-yl)phenyl)-2- hydroxyethane-1-sulfonamide |
| Compound 36 | 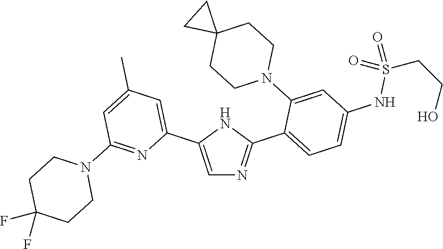 [SMILES: Cc1cc(-c2cnc(-c3ccc(NS(=O)(=O)CCO)cc3N3CCC4(CC3)CC4)[nH]2)nc(N2CCC(F)(F)CC2)c1] | N-(4-(5-(6-(4,4-difluoropiperidin-1-yl)-4- methylpyridin-2-yl)-1H-imidazol-2-yl)-3- (6-azaspiro[2.5]octan-6-yl)phenyl)-2- hydroxyethane-1-sulfonamide |
| Compound 37 | 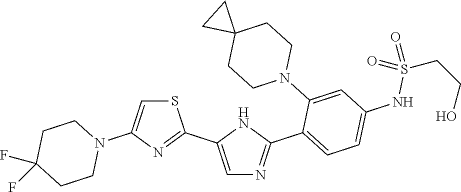 [SMILES: O=S(=O)(CCO)Nc1ccc(-c2ncc(-c3nc(N4CCC(F)(F)CC4)cs3)[nH]2)c(N2CCC3(CC2)CC3)c1] | N-(4-(5-(4-(4,4-difluoropiperidin-1- yl)thiazol-2-yl)-1H-imidazol-2-yl)-3-(6- azaspiro[2.5]octan-6-yl)phenyl)-2- hydroxyethane-1-sulfonamide |
| Compound 38 | 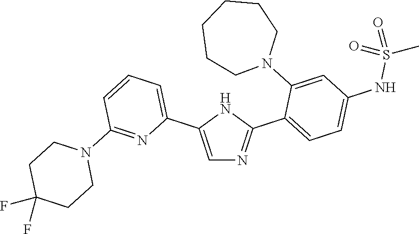 [SMILES: CS(=O)(=O)Nc1ccc(-c2ncc(-c3cccc(N4CCC(F)(F)CC4)n3)[nH]2)c(N2CCCCCC2)c1] | N-(3-(azepan-1-yl)-4-(5-(6-(4,4- difluoropiperidin-1-yl)pyridin-2-yl)-1H- imidazol-2-yl)phenyl)methanesulfonamide |
| Compound 39 | 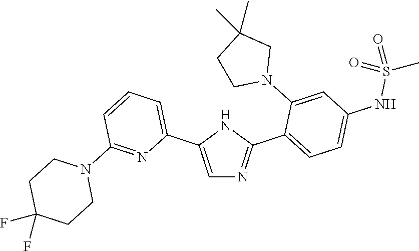 [SMILES: CC1(C)CCN(c2cc(NS(C)(=O)=O)ccc2-c2ncc(-c3cccc(N4CCC(F)(F)CC4)n3)[nH]2)C1] | N-(4-(5-(6-(4,4-difluoropiperidin-1- yl)pyridin-2-yl)-1H-imidazol-2-yl)-3-(3,3- dimethylpyrrolidin-1- yl)phenyl)methanesulfonamide |
| Compound 40 | 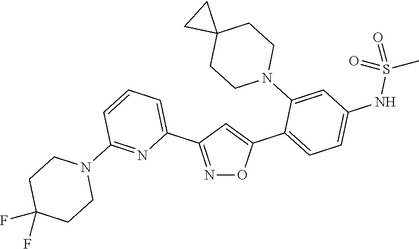 [SMILES: CS(=O)(=O)Nc1ccc(-c2cc(-c3cccc(N4CCC(F)(F)CC4)n3)no2)c(N2CCC3(CC2)CC3)c1] | N-(4-(5-(6-(4,4-difluoropiperidin-1- yl)pyridin-2-yl)isoxazol-3-yl)-3-(6- azaspiro[2.5]octan-6- yl)phenyl)methanesulfonamide |
| Compound 41 | 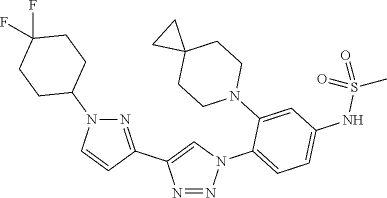 [SMILES: CS(=O)(=O)Nc1ccc(-n2cc(-c3ccn(C4CCC(F)(F)CC4)n3)nn2)c(N2CCC3(CC2)CC3)c1] | N-(4-(4-(1-(4,4-difluorocyclohexyl)-1H- pyrazol-3-yl)-1H-1,2,3-triazol-1-yl)-3-(6- azaspiro[2.5]octan-6- yl)phenyl)methanesulfonamide |
| Compound 42 | 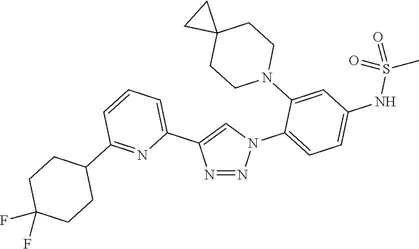 [SMILES: CS(=O)(=O)Nc1ccc(-n2cc(-c3cccc(C4CCC(F)(F)CC4)n3)nn2)c(N2CCC3(CC2)CC3)c1] | N-(4-(4-(6-(4,4- difluorocyclohexyl)pyridin-2-yl)-1H- 1,2,3-triazol-1-yl)-3-(6-azaspiro[2.5]octan- 6-yl)phenyl)methanesulfonamide |
| Compound 45 | 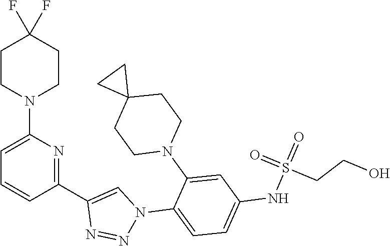 [SMILES: O=S(=O)(CCO)Nc1ccc(-n2cc(-c3cccc(N4CCC(F)(F)CC4)n3)nn2)c(N2CCC3(CC2)CC3)c1] | N-(4-(4-(6-(4,4-difluoropiperidin-1- yl)pyridin-2-yl)-1H-1,2,3-triazol-1-yl)-3- (6-azaspiro[2.5]octan-6-yl)phenyl)-2- hydroxyethane-1-sulfonamide |
| Compound 46 | 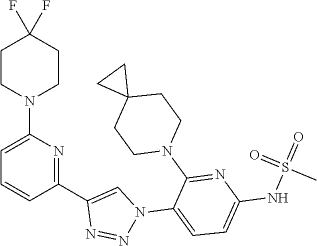 [SMILES: CS(=O)(=O)Nc1ccc(-n2cc(-c3cccc(N4CCC(F)(F)CC4)n3)nn2)c(N2CCC3(CC2)CC3)n1] | N-(5-(4-(6-(4,4-difluoropiperidin-1- yl)pyridin-2-yl)-1H-1,2,3-triazol-1-yl)-6- (6-azaspiro[2.5]octan-6-yl)pyridin-2- yl)methanesulfonamide |
| Compound 47 | 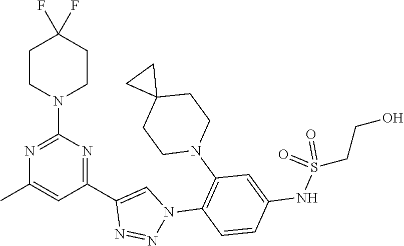 [SMILES: Cc1cc(-c2cn(-c3ccc(NS(=O)(=O)CCO)cc3N3CCC4(CC3)CC4)nn2)nc(N2CCC(F)(F)CC2)n1] | N-(4-(4-(2-(4,4-difluoropiperidin-1-yl)-6- methylpyrimidin-4-yl)-1H-1,2,3-triazol-1- yl)-3-(6-azaspiro[2.5]octan-6-yl)phenyl)- 2-hydroxyethane-1-sulfonamide |
| Compound 48 | 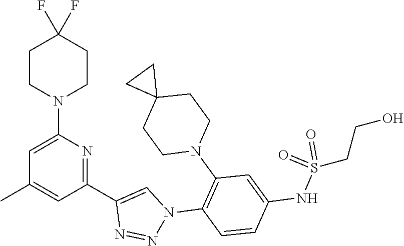 [SMILES: Cc1cc(-c2cn(-c3ccc(NS(=O)(=O)CCO)cc3N3CCC4(CC3)CC4)nn2)nc(N2CCC(F)(F)CC2)c1] | N-(4-(4-(6-(4,4-difluoropiperidin-1-yl)-4- methylpyridin-2-yl)-1H-1,2,3-triazol-1- yl)-3-(6-azaspiro[2.5]octan-6-yl)phenyl)- 2-hydroxyethane-1-sulfonamide |
| Compound 49 | 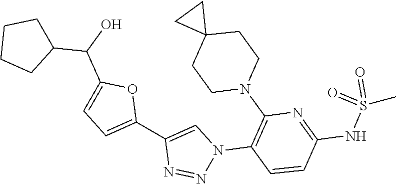 [SMILES: CS(=O)(=O)Nc1ccc(-n2cc(-c3ccc(C(O)C4CCCC4)o3)nn2)c(N2CCC3(CC2)CC3)n1] | N-(5-(4-(5- (cyclopentyl(hydroxy)methyl)furan-2-yl)- 1H-1,2,3-triazol-1-yl)-6-(6- azaspiro[2.5]octan-6-yl)pyridin-2- yl)methanesulfonamide |
| Compound 50 | 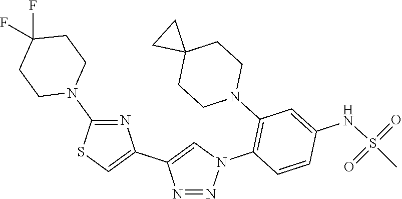 [SMILES: CS(=O)(=O)Nc1ccc(-n2cc(-c3csc(N4CCC(F)(F)CC4)n3)nn2)c(N2CCC3(CC2)CC3)c1] | N-(4-(4-(2-(4,4-difluoropiperidin-1- yl)thiazol-4-yl)-1H-1,2,3-triazol-1-yl)-3- (6-azaspiro[2.5]octan-6- yl)phenyl)methanesulfonamide |
| Compound 51 | 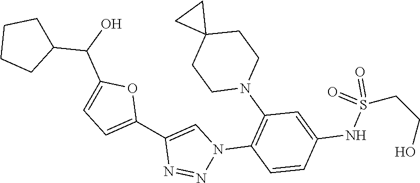 [SMILES: O=S(=O)(CCO)Nc1ccc(-n2cc(-c3ccc(C(O)C4CCCC4)o3)nn2)c(N2CCC3(CC2)CC3)c1] | N-(4-(4-(5- (cyclopentyl(hydroxy)methyl)furan-2-yl)- 1H-1,2,3-triazol-1-yl)-3-(6- azaspiro[2.5]octan-6-yl)phenyl)-2- hydroxyethane-1-sulfonamide |
| (R)- Compound 51 | 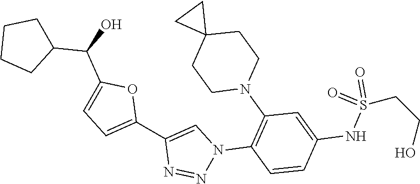 [SMILES: O=S(=O)(CCO)Nc1ccc(-n2cc(-c3ccc([C@H](O)C4CCCC4)o3)nn2)c(N2CCC3(CC2)CC3)c1] | (R)-N-(4-(4-(5- (cyclopentyl(hydroxy)methyl)furan-2-yl)- 1H-1,2,3-triazol-1-yl)-3-(6- azaspiro[2.5]octan-6-yl)phenyl)-2- hydroxyethane-1-sulfonamide |
| (S)- Compound 51 | 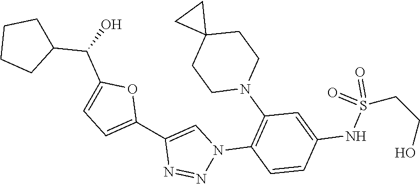 [SMILES: O=S(=O)(CCO)Nc1ccc(-n2cc(-c3ccc([C@@H](O)C4CCCC4)o3)nn2)c(N2CCC3(CC2)CC3)c1] | (S)-N-(4-(4-(5- (cyclopentyl(hydroxy)methyl)furan-2-yl)- 1H-1,2,3-triazol-1-yl)-3-(6- azaspiro[2.5]octan-6-yl)phenyl)-2- hydroxyethane-1-sulfonamide |
| Compound 52 | 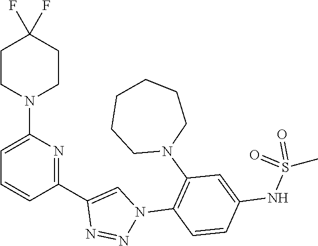 [SMILES: CS(=O)(=O)Nc1ccc(-n2cc(-c3cccc(N4CCC(F)(F)CC4)n3)nn2)c(N2CCCCCC2)c1] | N-(3-(azepan-1-yl)-4-(4-(6-(4,4- difluoropiperidin-1-yl)pyridin-2-yl)-1H- 1,2,3-triazol-1- yl)phenyl)methanesulfonamide |
| Compound 53 | 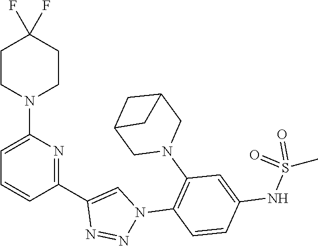 [SMILES: CS(=O)(=O)Nc1ccc(-n2cc(-c3cccc(N4CCC(F)(F)CC4)n3)nn2)c(N2CC3CC(C3)C2)c1] | N-(3-(3-azabicyclo[3.1.1]heptan-3-yl)-4- (4-(6-(4,4-difluoropiperidin-1-yl)pyridin- 2-yl)-1H-1,2,3-triazol-1- yl)phenyl)methanesulfonamide |
| Compound 54 | 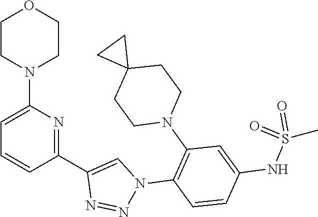 [SMILES: CS(=O)(=O)Nc1ccc(-n2cc(-c3cccc(N4CCOCC4)n3)nn2)c(N2CCC3(CC2)CC3)c1] | N-(4-(4-(6-morpholinopyridin-2-yl)-1H- 1,2,3-triazol-1-yl)-3-(6-azaspiro[2.5]octan- 6-yl)phenyl)methanesulfonamide |
| Compound 55 | 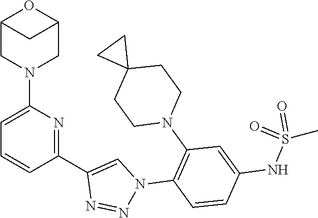 [SMILES: CS(=O)(=O)Nc1ccc(-n2cc(-c3cccc(N4CC5CC(C4)O5)n3)nn2)c(N2CCC3(CC2)CC3)c1] | N-(4-(4-(6-(6-oxa-3- azabicyclo[3.1.1]heptan-3-yl)pyridin-2- yl)-1H-1,2,3-triazol-1-yl)-3-(6- azaspiro[2.5]octan-6- yl)phenyl)methanesulfonamide |
| Compound 56 | 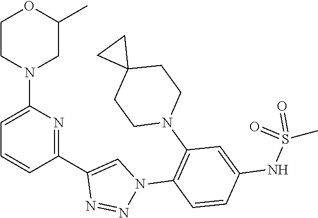 [SMILES: CC1CN(c2cccc(-c3cn(-c4ccc(NS(C)(=O)=O)cc4N4CCC5(CC4)CC5)nn3)n2)CCO1] | N-(4-(4-(6-(2-methylmorpholino)pyridin- 2-yl)-1H-1,2,3-triazol-1-yl)-3-(6- azaspiro[2.5]octan-6- yl)phenyl)methanesulfonamide |
| Compound 57 | 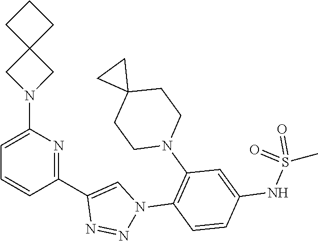 [SMILES: CS(=O)(=O)Nc1ccc(-n2cc(-c3cccc(N4CC5(CCC5)C4)n3)nn2)c(N2CCC3(CC2)CC3)c1] | N-(4-(4-(6-(2-azaspiro[3.3]heptan-2- yl)pyridin-2-yl)-1H-1,2,3-triazol-1-yl)-3- (6-azaspiro[2.5]octan-6- yl)phenyl)methanesulfonamide |
| Compound 58 | 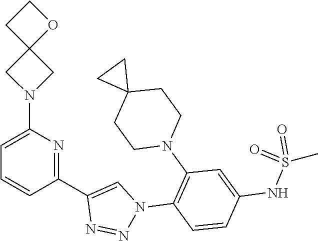 [SMILES: CS(=O)(=O)Nc1ccc(-n2cc(-c3cccc(N4CC5(CCO5)C4)n3)nn2)c(N2CCC3(CC2)CC3)c1] | N-(4-(4-(6-(1-oxa-6-azaspiro[3.3]heptan- 6-yl)pyridin-2-yl)-1H-1,2,3-triazol-1-yl)- 3-(6-azaspiro[2.5]octan-6- yl)phenyl)methanesulfonamide |
| Compound 59 | 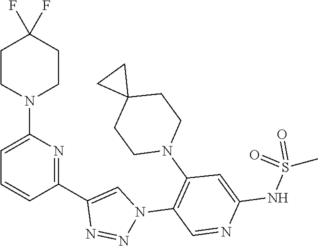 [SMILES: CS(=O)(=O)Nc1cc(N2CCC3(CC2)CC3)c(-n2cc(-c3cccc(N4CCC(F)(F)CC4)n3)nn2)cn1] | N-(5-(4-(6-(4,4-difluoropiperidin-1- yl)pyridin-2-yl)-1H-1,2,3-triazol-1-yl)-4- (6-azaspiro[2.5]octan-6-yl)pyridin-2- yl)methanesulfonamide |
| Compound 60 | 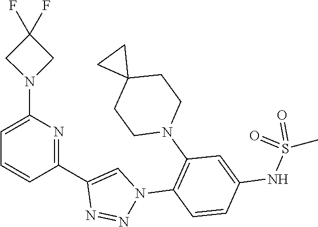 [SMILES: CS(=O)(=O)Nc1ccc(-n2cc(-c3cccc(N4CC(F)(F)C4)n3)nn2)c(N2CCC3(CC2)CC3)c1] | N-(4-(4-(6-(3,3-difluoroazetidin-1- yl)pyridin-2-yl)-1H-1,2,3-triazol-1-yl)-3- (6-azaspiro[2.5]octan-6- yl)phenyl)methanesulfonamide |
| Compound 61 | 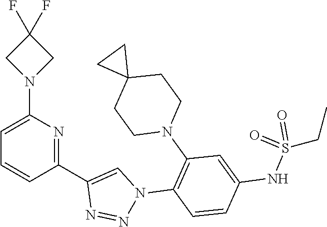 [SMILES: CCS(=O)(=O)Nc1ccc(-n2cc(-c3cccc(N4CC(F)(F)C4)n3)nn2)c(N2CCC3(CC2)CC3)c1] | N-(4-(4-(6-(3,3-difluoroazetidin-1- yl)pyridin-2-yl)-1H-1,2,3-triazol-1-yl)-3- (6-azaspiro[2.5]octan-6- yl)phenyl)ethanesulfonamide |
| Compound 62 | 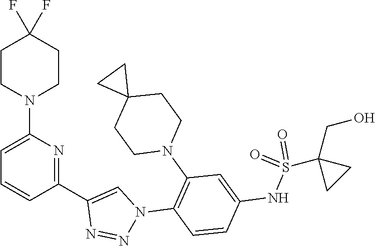 [SMILES: O=S(=O)(Nc1ccc(-n2cc(-c3cccc(N4CCC(F)(F)CC4)n3)nn2)c(N2CCC3(CC2)CC3)c1)C1(CO)CC1] | N-(4-(4-(6-(4,4-difluoropiperidin-1- yl)pyridin-2-yl)-1H-1,2,3-triazol-1-yl)-3- (6-azaspiro[2.5]octan-6-yl)phenyl)-1- (hydroxymethyl)cyclopropane-1- sulfonamide |
| Compound 63 | 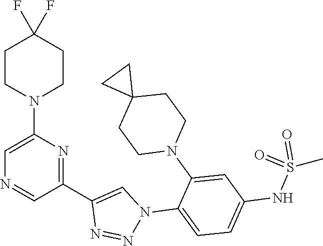 [SMILES: CS(=O)(=O)Nc1ccc(-n2cc(-c3cncc(N4CCC(F)(F)CC4)n3)nn2)c(N2CCC3(CC2)CC3)c1] | N-(4-(4-(6-(4,4-difluoropiperidin-1- yl)pyrazin-2-yl)-1H-1,2,3-triazol-1-yl)-3- (6-azaspiro[2.5]octan-6- yl)phenyl)methanesulfonamide |
| Compound 64 | 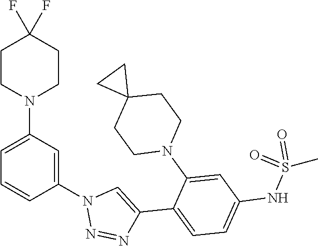 [SMILES: CS(=O)(=O)Nc1ccc(-c2cn(-c3cccc(N4CCC(F)(F)CC4)c3)nn2)c(N2CCC3(CC2)CC3)c1] | N-(4-(1-(3-(4,4-difluoropiperidin-1- yl)phenyl)-1H-1,2,3-triazol-4-yl)-3-(6- azaspiro[2.5]octan-6- yl)phenyl)methanesulfonamide |
| Compound 65 | 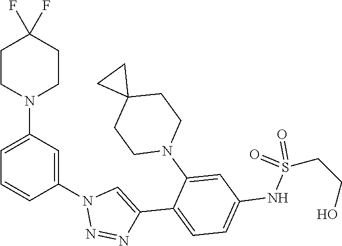 [SMILES: O=S(=O)(CCO)Nc1ccc(-c2cn(-c3cccc(N4CCC(F)(F)CC4)c3)nn2)c(N2CCC3(CC2)CC3)c1] | N-(4-(1-(3-(4,4-difluoropiperidin-1- yl)phenyl)-1H-1,2,3-triazol-4-yl)-3-(6- azaspiro[2.5]octan-6-yl)phenyl)-2- hydroxyethane-1-sulfonamide |
| Compound 66 | 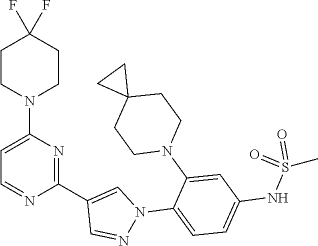 [SMILES: CS(=O)(=O)Nc1ccc(-n2cc(-c3nccc(N4CCC(F)(F)CC4)n3)cn2)c(N2CCC3(CC2)CC3)c1] | N-(4-(4-(4-(4,4-difluoropiperidin-1- yl)pyrimidin-2-yl)-1H-pyrazol-1-yl)-3-(6- azaspiro[2.5]octan-6- yl)phenyl)methanesulfonamide |
| Compound 67 | 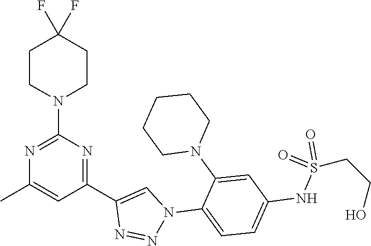 [SMILES: Cc1cc(-c2cn(-c3ccc(NS(=O)(=O)CCO)cc3N3CCCCC3)nn2)nc(N2CCC(F)(F)CC2)n1] | N-(4-(4-(2-(4,4-difluoropiperidin-1-yl)-6- methylpyrimidin-4-yl)-1H-1,2,3-triazol-1- yl)-3-(piperidin-1-yl)phenyl)-2- hydroxyethane-1-sulfonamide |
| Compound 68 | 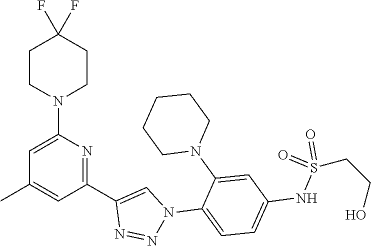 [SMILES: Cc1cc(-c2cn(-c3ccc(NS(=O)(=O)CCO)cc3N3CCCCC3)nn2)nc(N2CCC(F)(F)CC2)c1] | N-(4-(4-(6-(4,4-difluoropiperidin-1-yl)-4- methylpyridin-2-yl)-1H-1,2,3-triazol-1- yl)-3-(piperidin-1-yl)phenyl)-2- hydroxyethane-1-sulfonamide |
| Compound 69 | 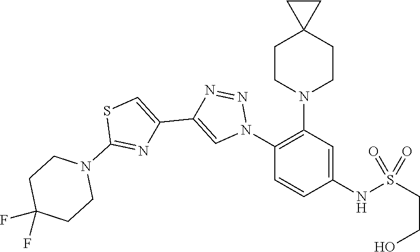 [SMILES: O=S(=O)(CCO)Nc1ccc(-n2cc(-c3csc(N4CCC(F)(F)CC4)n3)nn2)c(N2CCC3(CC2)CC3)c1] | N-(4-(4-(2-(4,4-difluoropiperidin-1- yl)thiazol-4-yl)-1H-1,2,3-triazol-1-yl)-3- (6-azaspiro[2.5]octan-6-yl)phenyl)-2- hydroxyethane-1-sulfonamide |
| Compound 70 | 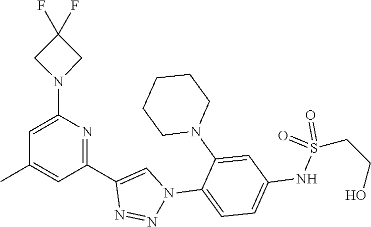 [SMILES: Cc1cc(-c2cn(-c3ccc(NS(=O)(=O)CCO)cc3N3CCCCC3)nn2)nc(N2CC(F)(F)C2)c1] | N-(4-(4-(6-(3,3-difluoroazetidin-1-yl)-4- methylpyridin-2-yl)-1H-1,2,3-triazol-1- yl)-3-(piperidin-1-yl)phenyl)-2- hydroxyethane-1-sulfonamide |
| Compound 71 | 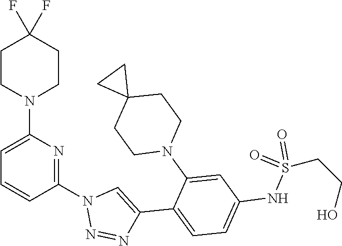 [SMILES: O=S(=O)(CCO)Nc1ccc(-c2cn(-c3cccc(N4CCC(F)(F)CC4)n3)nn2)c(N2CCC3(CC2)CC3)c1] | N-(4-(1-(6-(4,4-difluoropiperidin-1- yl)pyridin-2-yl)-1H-1,2,3-triazol-4-yl)-3- (6-azaspiro[2.5]octan-6-yl)phenyl)-2- hydroxyethane-1-sulfonamide |
| Compound 72 | 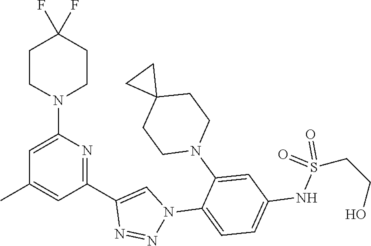 [SMILES: Cc1cc(-c2cn(-c3ccc(NS(=O)(=O)CCO)cc3N3CCC4(CC3)CC4)nn2)nc(N2CCC(F)(F)CC2)c1] | N-(4-(4-(6-(4,4-difluoropiperidin-1-yl)-4- methylpyridin-2-yl)-1H-1,2,3-triazol-1- yl)-3-(6-azaspiro[2.5]octan-6-yl)phenyl)- 2-hydroxyethane-1-sulfonamide |
| Compound 73 | 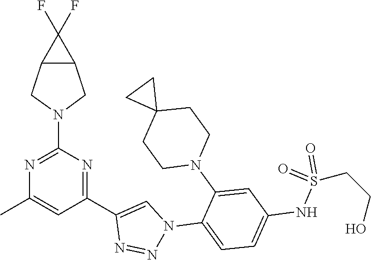 [SMILES: Cc1cc(-c2cn(-c3ccc(NS(=O)(=O)CCO)cc3N3CCC4(CC3)CC4)nn2)nc(N2CC3C(C2)C3(F)F)n1] | N-(4-(4-(2-(6,6-difluoro-3- azabicyclo[3.1.0]hexan-3-yl)-6- methylpyrimidin-4-yl)-1H-1,2,3-triazol-1- yl)-3-(6-azaspiro[2.5]octan-6-yl)phenyl)- 2-hydroxyethane-1-sulfonamide |
| Compound 74 | 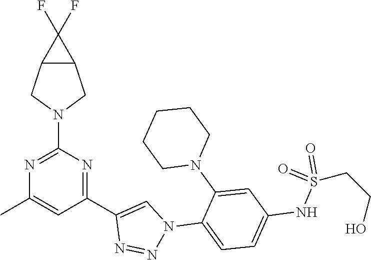 [SMILES: Cc1cc(-c2cn(-c3ccc(NS(=O)(=O)CCO)cc3N3CCCCC3)nn2)nc(N2CC3C(C2)C3(F)F)n1] | N-(4-(4-(2-(6,6-difluoro-3- azabicyclo[3.1.0]hexan-3-yl)-6- methylpyrimidin-4-yl)-1H-1,2,3-triazol-1- yl)-3-(piperidin-1-yl)phenyl)-2- hydroxyethane-1-sulfonamide |
| Compound 75 | 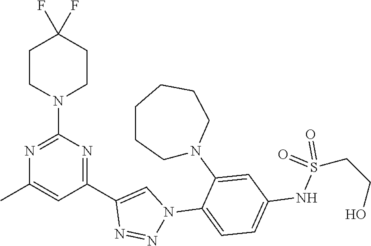 [SMILES: Cc1cc(-c2cn(-c3ccc(NS(=O)(=O)CCO)cc3N3CCCCCC3)nn2)nc(N2CCC(F)(F)CC2)n1] | N-(3-(azepan-1-yl)-4-(4-(2-(4,4- difluoropiperidin-1-yl)-6- methylpyrimidin-4-yl)-1H-1,2,3-triazol-1- yl)phenyl)-2-hydroxyethane-1- sulfonamide |
| Compound 76 | 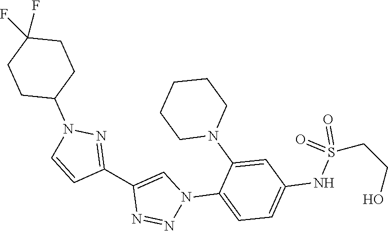 [SMILES: O=S(=O)(CCO)Nc1ccc(-n2cc(-c3ccn(C4CCC(F)(F)CC4)n3)nn2)c(N2CCCCC2)c1] | N-(4-(4-(1-(4,4-difluorocyclohexyl)-1H- pyrazol-3-yl)-1H-1,2,3-triazol-1-yl)-3- (piperidin-1-yl)phenyl)-2-hydroxyethane- 1-sulfonamide |
| Compound 77 | 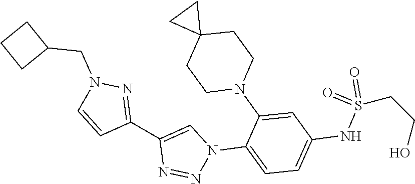 [SMILES: O=S(=O)(CCO)Nc1ccc(-n2cc(-c3ccn(CC4CCC4)n3)nn2)c(N2CCC3(CC2)CC3)c1] | N-(4-(4-(1-(cyclobutylmethyl)-1H- pyrazol-3-yl)-1H-1,2,3-triazol-1-yl)-3-(6- azaspiro[2.5]octan-6-yl)phenyl)-2- hydroxyethane-1-sulfonamide |
| Compound 78 | 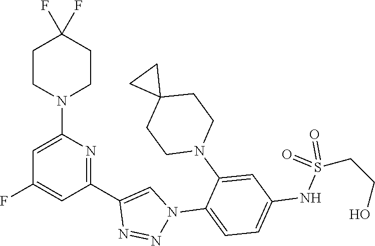 [SMILES: O=S(=O)(CCO)Nc1ccc(-n2cc(-c3cc(F)cc(N4CCC(F)(F)CC4)n3)nn2)c(N2CCC3(CC2)CC3)c1] | N-(4-(4-(6-(4,4-difluoropiperidin-1-yl)-4- fluoropyridin-2-yl)-1H-1,2,3-triazol-1-yl)- 3-(6-azaspiro[2.5]octan-6-yl)phenyl)-2- hydroxyethane-1-sulfonamide |
| Compound 79 | 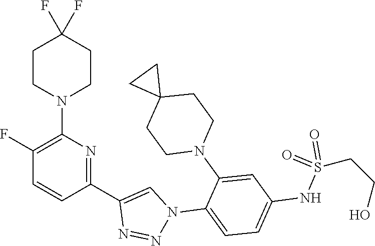 [SMILES: O=S(=O)(CCO)Nc1ccc(-n2cc(-c3ccc(F)c(N4CCC(F)(F)CC4)n3)nn2)c(N2CCC3(CC2)CC3)c1] | N-(4-(4-(6-(4,4-difluoropiperidin-1-yl)-5- fluoropyridin-2-yl)-1H-1,2,3-triazol-1-yl)- 3-(6-azaspiro[2.5]octan-6-yl)phenyl)-2- hydroxyethane-1-sulfonamide |
| Compound 80 | 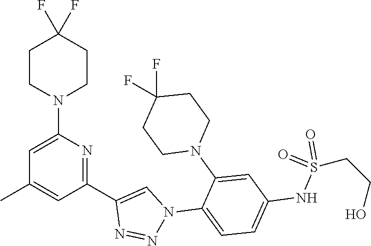 [SMILES: Cc1cc(-c2cn(-c3ccc(NS(=O)(=O)CCO)cc3N3CCC(F)(F)CC3)nn2)nc(N2CCC(F)(F)CC2)c1] | N-(3-(4,4-difluoropiperidin-1-yl)-4-(4-(6- (4,4-difluoropiperidin-1-yl)-4- methylpyridin-2-yl)-1H-1,2,3-triazol-1- yl)phenyl)-2-hydroxyethane-1- sulfonamide |
| Compound 81 | 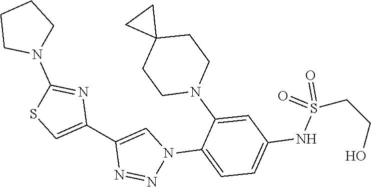 [SMILES: O=S(=O)(CCO)Nc1ccc(-n2cc(-c3csc(N4CCCC4)n3)nn2)c(N2CCC3(CC2)CC3)c1] | 2-hydroxy-N-(4-(4-(2-(pyrrolidin-1- yl)thiazol-4-yl)-1H-1,2,3-triazol-1-yl)-3- (6-azaspiro[2.5]octan-6-yl)phenyl)ethane- 1-sulfonamide |
| Compound 82 | 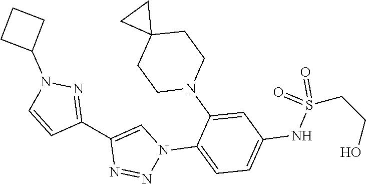 [SMILES: O=S(=O)(CCO)Nc1ccc(-n2cc(-c3ccn(C4CCC4)n3)nn2)c(N2CCC3(CC2)CC3)c1] | N-(4-(4-(1-cyclobutyl-1H-pyrazol-3-yl)- 1H-1,2,3-triazol-1-yl)-3-(6- azaspiro[2.5]octan-6-yl)phenyl)-2- hydroxyethane-1-sulfonamide |
| Compound 83 | 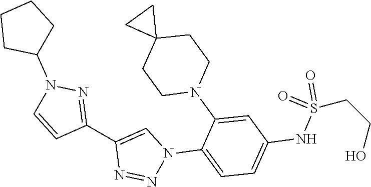 [SMILES: O=S(=O)(CCO)Nc1ccc(-n2cc(-c3ccn(C4CCCC4)n3)nn2)c(N2CCC3(CC2)CC3)c1] | N-(4-(4-(1-cyclopentyl-1H-pyrazol-3-yl)- 1H-1,2,3-triazol-1-yl)-3-(6- azaspiro[2.5]octan-6-yl)phenyl)-2- hydroxyethane-1-sulfonamide |
| Compound 84 | 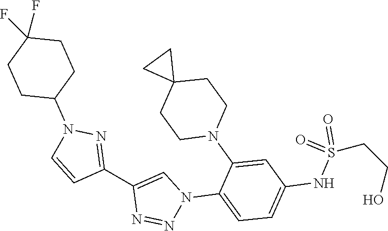 [SMILES: O=S(=O)(CCO)Nc1ccc(-n2cc(-c3ccn(C4CCC(F)(F)CC4)n3)nn2)c(N2CCC3(CC2)CC3)c1] | N-(4-(4-(1-(4,4-difluorocyclohexyl)-1H- pyrazol-3-yl)-1H-1,2,3-triazol-1-yl)-3-(6- azaspiro[2.5]octan-6-yl)phenyl)-2- hydroxyethane-1-sulfonamide |
| Compound 85 | 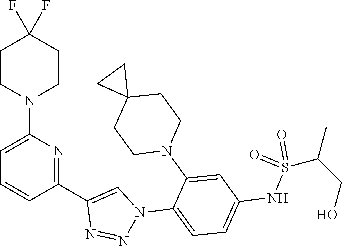 [SMILES: CC(CO)S(=O)(=O)Nc1ccc(-n2cc(-c3cccc(N4CCC(F)(F)CC4)n3)nn2)c(N2CCC3(CC2)CC3)c1] | N-(4-(4-(6-(4,4-difluoropiperidin-1- yl)pyridin-2-yl)-1H-1,2,3-triazol-1-yl)-3- (6-azaspiro[2.5]octan-6-yl)phenyl)-1- hydroxypropane-2-sulfonamide |
| Compound 86 | 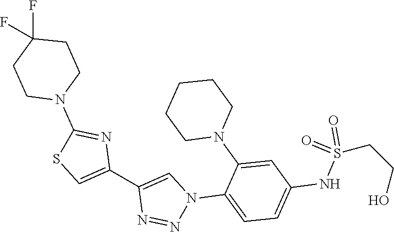 [SMILES: O=S(=O)(CCO)Nc1ccc(-n2cc(-c3csc(N4CCC(F)(F)CC4)n3)nn2)c(N2CCCCC2)c1] | N-(4-(4-(2-(4,4-difluoropiperidin-1- yl)thiazol-4-yl)-1H-1,2,3-triazol-1-yl)-3- (piperidin-1-yl)phenyl)-2-hydroxyethane- 1-sulfonamide |
| Compound 87 | 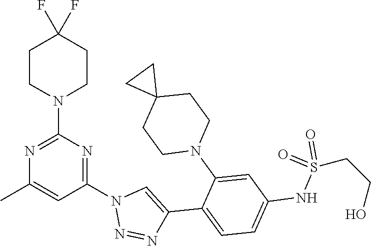 [SMILES: Cc1cc(-n2cc(-c3ccc(NS(=O)(=O)CCO)cc3N3CCC4(CC3)CC4)nn2)nc(N2CCC(F)(F)CC2)n1] | N-(4-(1-(2-(4,4-difluoropiperidin-1-yl)-6- methylpyrimidin-4-yl)-1H-1,2,3-triazol-4- yl)-3-(6-azaspiro[2.5]octan-6-yl)phenyl)- 2-hydroxyethane-1-sulfonamide |
| Compound 88 | 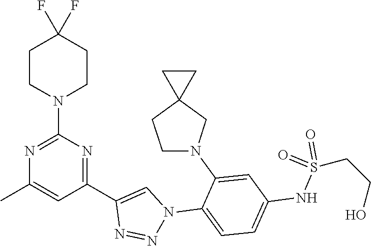 [SMILES: Cc1cc(-c2cn(-c3ccc(NS(=O)(=O)CCO)cc3N3CCC4(CC4)C3)nn2)nc(N2CCC(F)(F)CC2)n1] | N-(4-(4-(2-(4,4-difluoropiperidin-1-yl)-6- methylpyrimidin-4-yl)-1H-1,2,3-triazol-1- yl)-3-(5-azaspiro[2.4]heptan-5-yl)phenyl)- 2-hydroxyethane-1-sulfonamide |
| Compound 89 | 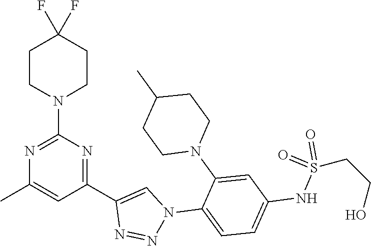 [SMILES: Cc1cc(-c2cn(-c3ccc(NS(=O)(=O)CCO)cc3N3CCC(C)CC3)nn2)nc(N2CCC(F)(F)CC2)n1] | N-(4-(4-(2-(4,4-difluoropiperidin-1-yl)-6- methylpyrimidin-4-yl)-1H-1,2,3-triazol-1- yl)-3-(4-methylpiperidin-1-yl)phenyl)-2- hydroxyethane-1-sulfonamide |
| Compound 90 | 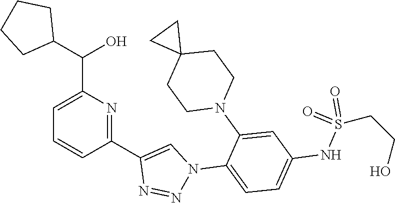 [SMILES: O=S(=O)(CCO)Nc1ccc(-n2cc(-c3cccc(C(O)C4CCCC4)n3)nn2)c(N2CCC3(CC2)CC3)c1] | N-(4-(4-(6- (cyclopentyl(hydroxy)methyl)pyridin-2- yl)-1H-1,2,3-triazol-1-yl)-3-(6- azaspiro[2.5]octan-6-yl)phenyl)-2- hydroxyethane-1-sulfonamide |
| (R)- Compound 90 | 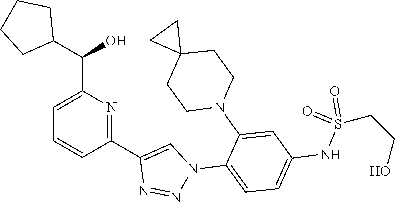 [SMILES: O=S(=O)(CCO)Nc1ccc(-n2cc(-c3cccc([C@H](O)C4CCCC4)n3)nn2)c(N2CCC3(CC2)CC3)c1] | (R)-N-(4-(4-(6- (cyclopentyl(hydroxy)methyl)pyridin-2- yl)-1H-1,2,3-triazol-1-yl)-3-(6- azaspiro[2.5]octan-6-yl)phenyl)-2- hydroxyethane-1-sulfonamide |
| (S)- Compound 90 | 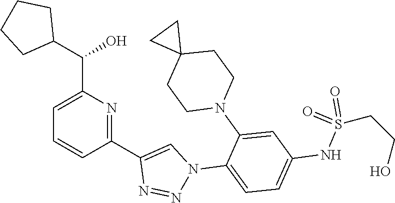 [SMILES: O=S(=O)(CCO)Nc1ccc(-n2cc(-c3cccc([C@@H](O)C4CCCC4)n3)nn2)c(N2CCC3(CC2)CC3)c1] | (S)-N-(4-(4-(6- (cyclopentyl(hydroxy)methyl)pyridin-2- yl)-1H-1,2,3-triazol-1-yl)-3-(6- azaspiro[2.5]octan-6-yl)phenyl)-2- hydroxyethane-1-sulfonamide |
| Compound 91 | 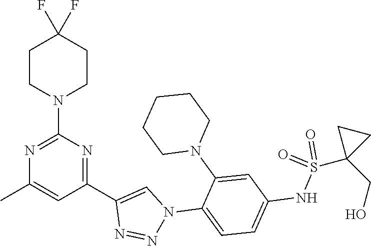 [SMILES: Cc1cc(-c2cn(-c3ccc(NS(=O)(=O)C4(CO)CC4)cc3N3CCCCC3)nn2)nc(N2CCC(F)(F)CC2)n1] | N-(4-(4-(2-(4,4-difluoropiperidin-1-yl)-6- methylpyrimidin-4-yl)-1H-1,2,3-triazol-1- yl)-3-(piperidin-1-yl)phenyl)-1- (hydroxymethyl)cyclopropane-1- sulfonamide |
| Compound 92 | 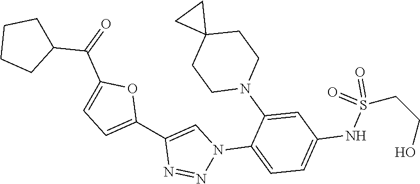 [SMILES: O=C(c1ccc(-c2cn(-c3ccc(NS(=O)(=O)CCO)cc3N3CCC4(CC3)CC4)nn2)o1)C1CCCC1] | N-(4-(4-(5-(cyclopentanecarbonyl)furan- 2-yl)-1H-1,2,3-triazol-1-yl)-3-(6- azaspiro[2.5]octan-6-yl)phenyl)-2- hydroxyethane-1-sulfonamide |
| Compound 93 | 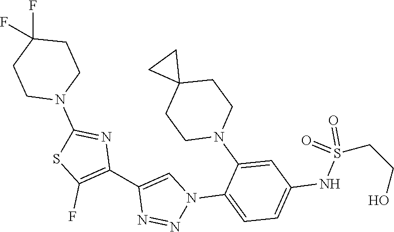 [SMILES: O=S(=O)(CCO)Nc1ccc(-n2cc(-c3nc(N4CCC(F)(F)CC4)sc3F)nn2)c(N2CCC3(CC2)CC3)c1] | N-(4-(4-(2-(4,4-difluoropiperidin-1-yl)-5- fluorothiazol-4-yl)-1H-1,2,3-triazol-1-yl)- 3-(6-azaspiro[2.5]octan-6-yl)phenyl)-2- hydroxyethane-1-sulfonamide |
| Compound 94 | 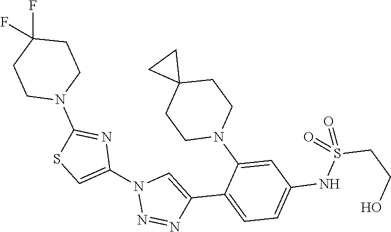 [SMILES: O=S(=O)(CCO)Nc1ccc(-c2cn(-c3csc(N4CCC(F)(F)CC4)n3)nn2)c(N2CCC3(CC2)CC3)c1] | N-(4-(4-(2-(4,4-difluoropiperidin-1- yl)thiazol-4-yl)-1H-1,2,3-triazol-1-yl)-3- (6-azaspiro[2.5]octan-6-yl)phenyl)-2- hydroxyethane-1-sulfonamide |
| Compound 95 | 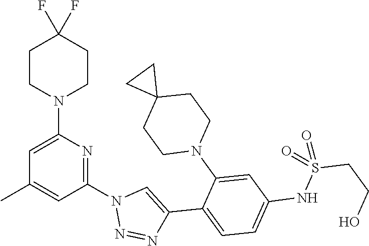 [SMILES: Cc1cc(N2CCC(F)(F)CC2)nc(-n2cc(-c3ccc(NS(=O)(=O)CCO)cc3N3CCC4(CC3)CC4)nn2)c1] | N-(4-(1-(6-(4,4-difluoropiperidin-1-yl)-4- methylpyridin-2-yl)-1H-1,2,3-triazol-4- yl)-3-(6-azaspiro[2.5]octan-6-yl)phenyl)- 2-hydroxyethane-1-sulfonamide |
| Compound 96 | 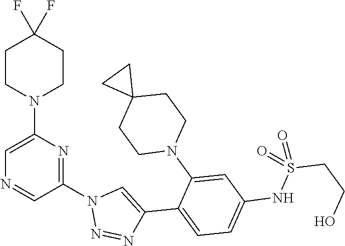 [SMILES: O=S(=O)(CCO)Nc1ccc(-c2cn(-c3cncc(N4CCC(F)(F)CC4)n3)nn2)c(N2CCC3(CC2)CC3)c1] | N-(4-(1-(6-(4,4-difluoropiperidin-1- yl)pyrazin-2-yl)-1H-1,2,3-triazol-4-yl)-3- (6-azaspiro[2.5]octan-6-yl)phenyl)-2- hydroxyethane-1-sulfonamide |
| Compound 97 | 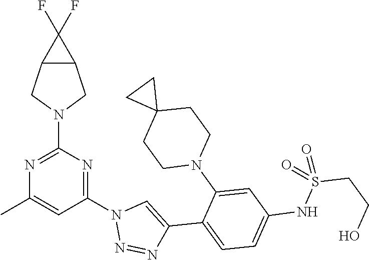 [SMILES: Cc1cc(-n2cc(-c3ccc(NS(=O)(=O)CCO)cc3N3CCC4(CC3)CC4)nn2)nc(N2CC3C(C2)C3(F)F)n1] | N-(4-(1-(2-(6,6-difluoro-3- azabicyclo[3.1.0]hexan-3-yl)-6- methylpyrimidin-4-yl)-1H-1,2,3-triazol-4- yl)-3-(6-azaspiro[2.5]octan-6-yl)phenyl)- 2-hydroxyethane-1-sulfonamide |
| Compound 98 | 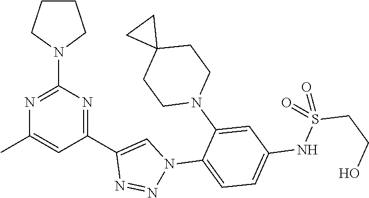 [SMILES: Cc1cc(-c2cn(-c3ccc(NS(=O)(=O)CCO)cc3N3CCC4(CC3)CC4)nn2)nc(N2CCCC2)n1] | 2-hydroxy-N-(4-(4-(6-methyl-2- (pyrrolidin-1-yl)pyrimidin-4-yl)-1H-1,2,3- triazol-1-yl)-3-(6-azaspiro[2.5]octan-6- yl)phenyl)ethane-1-sulfonamide |
| Compound 99 | 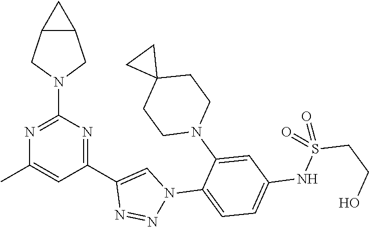 [SMILES: Cc1cc(-c2cn(-c3ccc(NS(=O)(=O)CCO)cc3N3CCC4(CC3)CC4)nn2)nc(N2CC3CC3C2)n1] | N-(4-(4-(2-(3-azabicyclo[3.1.0]hexan-3- yl)-6-methylpyrimidin-4-yl)-1H-1,2,3- triazol-1-yl)-3-(6-azaspiro[2.5]octan-6- yl)phenyl)-2-hydroxyethane-1- sulfonamide |
| Compound 100 | 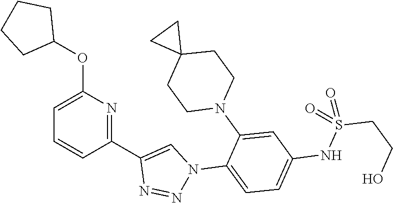 [SMILES: O=S(=O)(CCO)Nc1ccc(-n2cc(-c3cccc(OC4CCCC4)n3)nn2)c(N2CCC3(CC2)CC3)c1] | N-(4-(4-(6-(cyclopentyloxy)pyridin-2-yl)- 1H-1,2,3-triazol-1-yl)-3-(6- azaspiro[2.5]octan-6-yl)phenyl)-2- hydroxyethane-1-sulfonamide |
| Compound 101 | 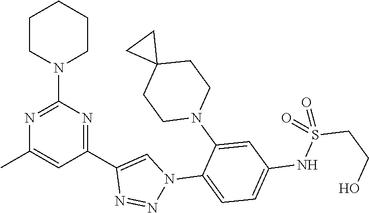 [SMILES: Cc1cc(-c2cn(-c3ccc(NS(=O)(=O)CCO)cc3N3CCC4(CC3)CC4)nn2)nc(N2CCCCC2)n1] | 2-hydroxy-N-(4-(4-(6-methyl-2- (piperidin-1-yl)pyrimidin-4-yl)-1H-1,2,3- triazol-1-yl)-3-(6-azaspiro[2.5]octan-6- yl)phenyl)ethane-1-sulfonamide |
| Compound 102 | 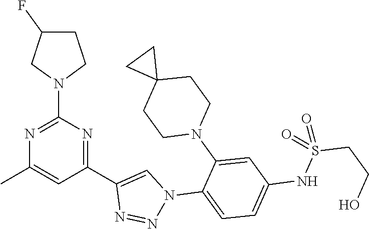 [SMILES: Cc1cc(-c2cn(-c3ccc(NS(=O)(=O)CCO)cc3N3CCC4(CC3)CC4)nn2)nc(N2CCC(F)C2)n1] | N-(4-(4-(2-(3-fluoropyrrolidin-1-yl)-6- methylpyrimidin-4-yl)-1H-1,2,3-triazol-1- yl)-3-(6-azaspiro[2.5]octan-6-yl)phenyl)- 2-hydroxyethane-1-sulfonamide |
| Compound 103 | 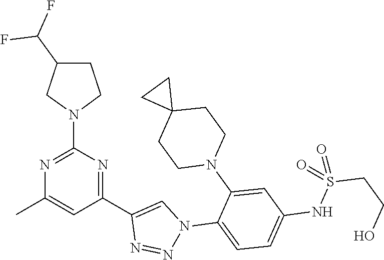 [SMILES: Cc1cc(-c2cn(-c3ccc(NS(=O)(=O)CCO)cc3N3CCC4(CC3)CC4)nn2)nc(N2CCC(C(F)F)C2)n1] | N-(4-(4-(2-(3-(difluoromethyl)pyrrolidin- 1-yl)-6-methylpyrimidin-4-yl)-1H-1,2,3- triazol-1-yl)-3-(6-azaspiro[2.5]octan-6- yl)phenyl)-2-hydroxyethane-1- sulfonamide |
| Compound 104 | 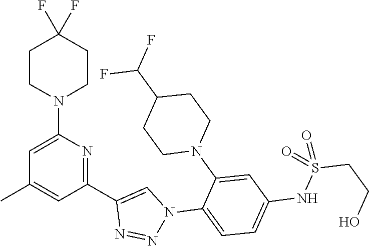 [SMILES: Cc1cc(-c2cn(-c3ccc(NS(=O)(=O)CCO)cc3N3CCC(C(F)F)CC3)nn2)nc(N2CCC(F)(F)CC2)c1] | N-(3-(4-(difluoromethyl)piperidin-1-yl)-4- (4-(6-(4,4-difluoropiperidin-1-yl)-4- methylpyridin-2-yl)-1H-1,2,3-triazol-1- yl)phenyl)-2-hydroxyethane-1- sulfonamide |
| Compound 105 | 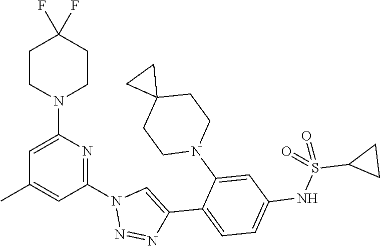 [SMILES: Cc1cc(N2CCC(F)(F)CC2)nc(-n2cc(-c3ccc(NS(=O)(=O)C4CC4)cc3N3CCC4(CC3)CC4)nn2)c1] | N-(4-(1-(6-(4,4-difluoropiperidin-1-yl)-4- methylpyridin-2-yl)-1H-1,2,3-triazol-4- yl)-3-(6-azaspiro[2.5]octan-6- yl)phenyl)cyclopropanesulfonamide |
| Compound 106 | 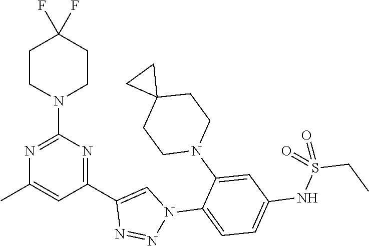 [SMILES: CCS(=O)(=O)Nc1ccc(-n2cc(-c3cc(C)nc(N4CCC(F)(F)CC4)n3)nn2)c(N2CCC3(CC2)CC3)c1] | N-(4-(4-(2-(4,4-difluoropiperidin-1-yl)-6- methylpyrimidin-4-yl)-1H-1,2,3-triazol-1- yl)-3-(6-azaspiro[2.5]octan-6- yl)phenyl)ethanesulfonamide |
| Compound 107 | 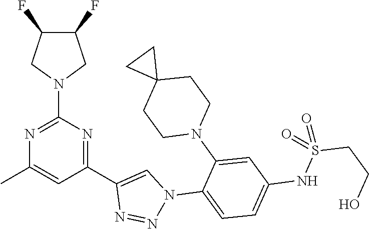 [SMILES: Cc1cc(-c2cn(-c3ccc(NS(=O)(=O)CCO)cc3N3CCC4(CC3)CC4)nn2)nc(N2C[C@@H](F)[C@@H](F)C2)n1] | N-(4-(4-(2-((3S,4R)-3,4- difluoropyrrolidin-1-yl)-6- methylpyrimidin-4-yl)-1H-1,2,3-triazol-1- yl)-3-(6-azaspiro[2.5]octan-6-yl)phenyl)- 2-hydroxyethane-1-sulfonamide |
| Compound 108 | 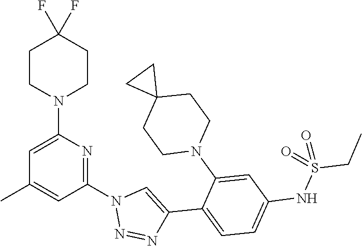 [SMILES: CCS(=O)(=O)Nc1ccc(-c2cn(-c3cc(C)cc(N4CCC(F)(F)CC4)n3)nn2)c(N2CCC3(CC2)CC3)c1] | N-(4-(1-(6-(4,4-difluoropiperidin-1-yl)-4- methylpyridin-2-yl)-1H-1,2,3-triazol-4- yl)-3-(6-azaspiro[2.5]octan-6- yl)phenyl)ethanesulfonamide |
| Compound 109 | 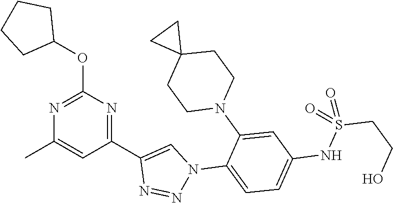 [SMILES: Cc1cc(-c2cn(-c3ccc(NS(=O)(=O)CCO)cc3N3CCC4(CC3)CC4)nn2)nc(OC2CCCC2)n1] | N-(4-(4-(2-(cyclopentyloxy)-6- methylpyrimidin-4-yl)-1H-1,2,3-triazol-1- yl)-3-(6-azaspiro[2.5]octan-6-yl)phenyl)- 2-hydroxyethane-1-sulfonamide |
| Compound 110 | 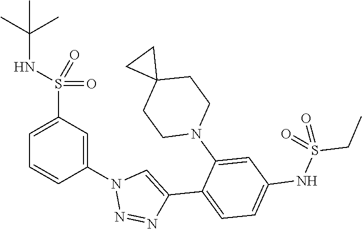 [SMILES: CCS(=O)(=O)Nc1ccc(-c2cn(-c3cccc(S(=O)(=O)NC(C)(C)C)c3)nn2)c(N2CCC3(CC2)CC3)c1] | N-(tert-butyl)-3-(4-(4-(ethylsulfonamido)- 2-(6-azaspiro[2.5]octan-6-yl)phenyl)-1H- 1,2,3-triazol-1-yl)benzenesulfonamide |
| Compound 111 | 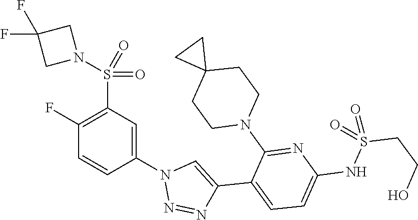 [SMILES: O=S(=O)(CCO)Nc1ccc(-c2cn(-c3ccc(F)c(S(=O)(=O)N4CC(F)(F)C4)c3)nn2)c(N2CCC3(CC2)CC3)n1] | N-(5-(1-(3-((3,3-difluoroazetidin-1- yl)sulfonyl)-4-fluorophenyl)-1H-1,2,3- triazol-4-yl)-6-(6-azaspiro[2.5]octan-6- yl)pyridin-2-yl)-2-hydroxyethane-1- sulfonamide |
| Compound 112 | 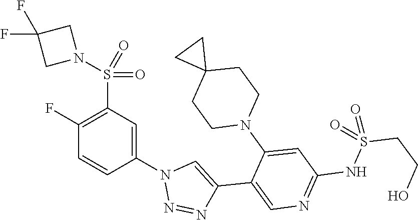 [SMILES: O=S(=O)(CCO)Nc1cc(N2CCC3(CC2)CC3)c(-c2cn(-c3ccc(F)c(S(=O)(=O)N4CC(F)(F)C4)c3)nn2)cn1] | N-(5-(1-(3-((3,3-difluoroazetidin-1- yl)sulfonyl)-4-fluorophenyl)-1H-1,2,3- triazol-4-yl)-(6-azaspiro[2.5]octan-6- yl)pyridin-2-yl)-2-hydroxyethane-1- sulfonamide |
| Compound 113 | 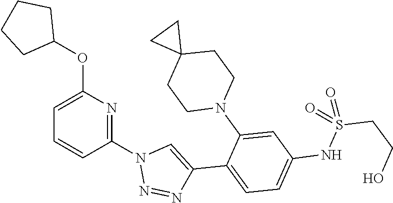 [SMILES: O=S(=O)(CCO)Nc1ccc(-c2cn(-c3cccc(OC4CCCC4)n3)nn2)c(N2CCC3(CC2)CC3)c1] | N-(4-(1-(6-(cyclopentyloxy)pyridin-2-yl)- 1H-1,2,3-triazol-4-yl)-3-(6- azaspiro[2.5]octan-6-yl)phenyl)-2- hydroxyethane-1-sulfonamide |
| Compound 114 | 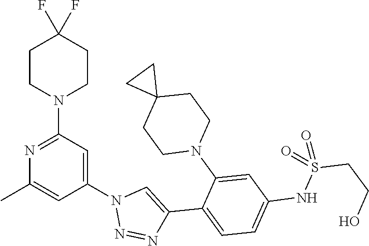 [SMILES: Cc1cc(-n2cc(-c3ccc(NS(=O)(=O)CCO)cc3N3CCC4(CC3)CC4)nn2)cc(N2CCC(F)(F)CC2)n1] | N-(4-(1-(2-(4,4-difluoropiperidin-1-yl)-6- methylpyridin-4-yl)-1H-1,2,3-triazol-4- yl)-3-(6-azaspiro[2.5]octan-6-yl)phenyl)- 2-hydroxyethane-1-sulfonamide |
| Compound 115 | 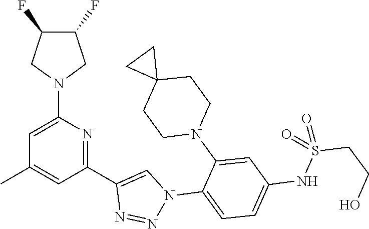 [SMILES: Cc1cc(-c2cn(-c3ccc(NS(=O)(=O)CCO)cc3N3CCC4(CC3)CC4)nn2)nc(N2C[C@@H](F)[C@H](F)C2)c1] | N-(4-(4-(6-((3R,4R)-3,4- difluoropyrrolidin-1-yl)-4-methylpyridin- 2-yl)-1H-1,2,3-triazol-1-yl)-3-(6- azaspiro[2.5]octan-6-yl)phenyl)-2- hydroxyethane-1-sulfonamide |
| Compound 116 | 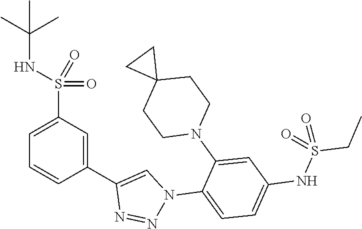 [SMILES: CCS(=O)(=O)Nc1ccc(-n2cc(-c3cccc(S(=O)(=O)NC(C)(C)C)c3)nn2)c(N2CCC3(CC2)CC3)c1] | N-(tert-butyl)-3-(1-(4-(ethylsulfonamido)- 2-(6-azaspiro[2.5]octan-6-yl)phenyl)-1H- 1,2,3-triazol-4-yl)benzenesulfonamide |
| Compound 117 | 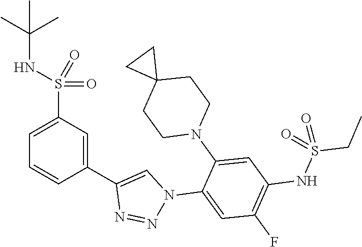 [SMILES: CCS(=O)(=O)Nc1cc(N2CCC3(CC2)CC3)c(-n2cc(-c3cccc(S(=O)(=O)NC(C)(C)C)c3)nn2)cc1F] | N-(tert-butyl)-3-(1-(4-(ethylsulfonamido)- 5-fluoro-2-(6-azaspiro[2.5]octan-6- yl)phenyl)-1H-1,2,3-triazol-4- yl)benzenesulfonamide |
| Compound 118 | 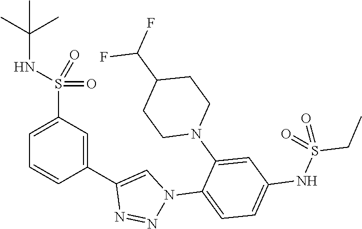 [SMILES: CCS(=O)(=O)Nc1ccc(-n2cc(-c3cccc(S(=O)(=O)NC(C)(C)C)c3)nn2)c(N2CCC(C(F)F)CC2)c1] | N-(tert-butyl)-3-(1-(2-(4- (difluoromethyl)piperidin-1-yl)-4- (ethylsulfonamido)phenyl)-1H-1,2,3- triazol-4-yl)benzenesulfonamide |
| Compound 119 | 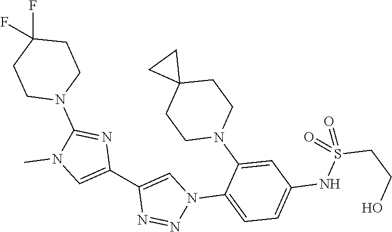 [SMILES: Cn1cc(-c2cn(-c3ccc(NS(=O)(=O)CCO)cc3N3CCC4(CC3)CC4)nn2)nc1N1CCC(F)(F)CC1] | N-(4-(4-(2-(4,4-difluoropiperidin-1-yl)-1- methyl-1H-imidazol-4-yl)-1H-1,2,3- triazol-1-yl)-3-(6-azaspiro[2.5]octan-6- yl)phenyl)-2-hydroxyethane-1- sulfonamide |
| Compound 120 | 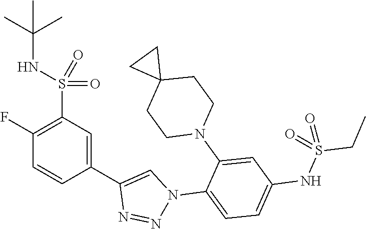 [SMILES: CCS(=O)(=O)Nc1ccc(-n2cc(-c3ccc(F)c(S(=O)(=O)NC(C)(C)C)c3)nn2)c(N2CCC3(CC2)CC3)c1] | N-(tert-butyl)-5-(1-(4-(ethylsulfonamido)- 2-(6-azaspiro[2.5]octan-6-yl)phenyl)-1H- 1,2,3-triazol-4-yl)-2- fluorobenzenesulfonamide |
| Compound 121 | 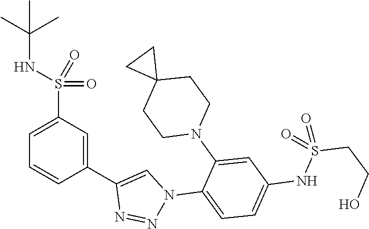 [SMILES: CC(C)(C)NS(=O)(=O)c1cccc(-c2cn(-c3ccc(NS(=O)(=O)CCO)cc3N3CCC4(CC3)CC4)nn2)c1] | N-(tert-butyl)-3-(1-(4-((2- hydroxyethyl)sulfonamido)-2-(6- azaspiro[2.5]octan-6-yl)phenyl)-1H-1,2,3- triazol-4-yl)benzenesulfonamide |
| Compound 122 | 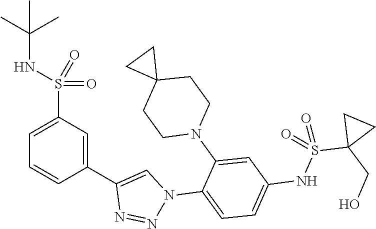 [SMILES: CC(C)(C)NS(=O)(=O)c1cccc(-c2cn(-c3ccc(NS(=O)(=O)C4(CO)CC4)cc3N3CCC4(CC3)CC4)nn2)c1] | N-(tert-butyl)-3-(1-(4-((1- (hydroxymethyl)cyclopropane)-1- sulfonamido)-2-(6-azaspiro[2.5]octan-6- yl)phenyl)-1H-1,2,3-triazol-4- yl)benzenesulfonamide |
| Compound 123 | 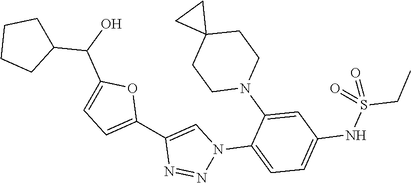 [SMILES: CCS(=O)(=O)Nc1ccc(-n2cc(-c3ccc(C(O)C4CCCC4)o3)nn2)c(N2CCC3(CC2)CC3)c1] | N-(4-(4-(5- (cyclopentyl(hydroxy)methyl)furan-2-yl)- 1H-1,2,3-triazol-1-yl)-3-(6- azaspiro[2.5]octan-6- yl)phenyl)ethanesulfonamide |
| Compound 124 | 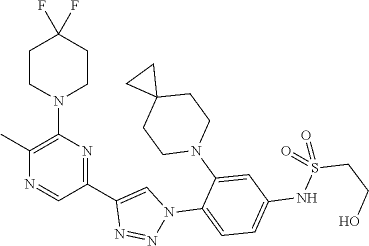 [SMILES: Cc1ncc(-c2cn(-c3ccc(NS(=O)(=O)CCO)cc3N3CCC4(CC3)CC4)nn2)nc1N1CCC(F)(F)CC1] | N-(4-(4-(6-(4,4-difluoropiperidin-1-yl)-5- methylpyrazin-2-yl)-1H-1,2,3-triazol-1- yl)-3-(6-azaspiro[2.5]octan-6-yl)phenyl)- 2-hydroxyethane-1-sulfonamide |
| Compound 125 | 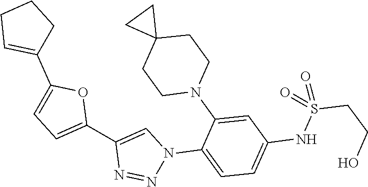 [SMILES: O=S(=O)(CCO)Nc1ccc(-n2cc(-c3ccc(C4=CCCC4)o3)nn2)c(N2CCC3(CC2)CC3)c1] | N-(4-(4-(5-(cyclopent-1-en-1-yl)furan-2- yl)-1H-1,2,3-triazol-1-yl)-3-(6- azaspiro[2.5]octan-6-yl)phenyl)-2- hydroxyethane-1-sulfonamide |
| Compound 126 | 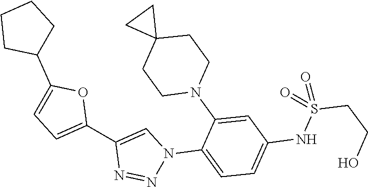 [SMILES: O=S(=O)(CCO)Nc1ccc(-n2cc(-c3ccc(C4CCCC4)o3)nn2)c(N2CCC3(CC2)CC3)c1] | N-(4-(4-(5-cyclopentylfuran-2-yl)-1H- 1,2,3-triazol-1-yl)-3-(6-azaspiro[2.5]octan- 6-yl)phenyl)-2-hydroxyethane-1- sulfonamide |
| Compound 127 | 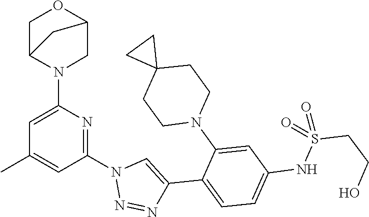 [SMILES: Cc1cc(N2CC3CC2CO3)nc(-n2cc(-c3ccc(NS(=O)(=O)CCO)cc3N3CCC4(CC3)CC4)nn2)c1] | N-(4-(1-(6-(2-oxa-5- azabicyclo[2.2.1]heptan-5-yl)-4- methylpyridin-2-yl)-1H-1,2,3-triazol-4- yl)-3-(6-azaspiro[2.5]octan-6-yl)phenyl)- 2-hydroxyethane-1-sulfonamide |
| (1R,4R)- Compound 127 | 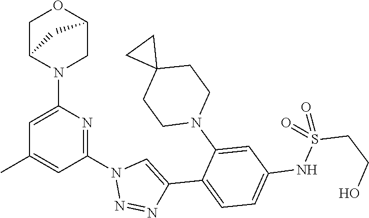 [SMILES: Cc1cc(N2C[C@H]3C[C@@H]2CO3)nc(-n2cc(-c3ccc(NS(=O)(=O)CCO)cc3N3CCC4(CC3)CC4)nn2)c1] | N-(4-(1-(6-((1R,4R)-2-oxa-5- azabicyclo[2.2.1]heptan-5-yl)-4- methylpyridin-2-yl)-1H-1,2,3-triazol-4- yl)-3-(6-azaspiro[2.5]octan-6-yl)phenyl)- 2-hydroxyethane-1-sulfonamide |
| (1S,4S)- Compound 127 | 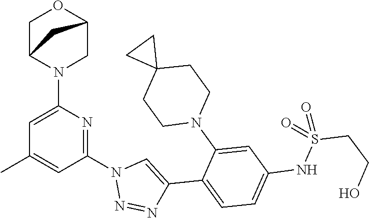 [SMILES: Cc1cc(N2C[C@@H]3C[C@H]2CO3)nc(-n2cc(-c3ccc(NS(=O)(=O)CCO)cc3N3CCC4(CC3)CC4)nn2)c1] | N-(4-(1-(6-((1S,4S)-2-oxa-5- azabicyclo[2.2.1]heptan-5-yl)-4- methylpyridin-2-yl)-1H-1,2,3-triazol-4- yl)-3-(6-azaspiro[2.5]octan-6-yl)phenyl)- 2-hydroxyethane-1-sulfonamide |
| Compound 128 | 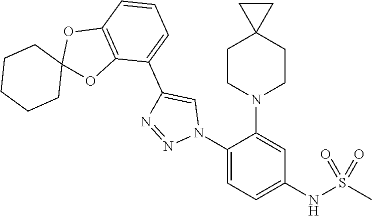 [SMILES: CS(=O)(=O)Nc1ccc(-n2cc(-c3cccc4c3OC3(CCCCC3)O4)nn2)c(N2CCC3(CC2)CC3)c1] | N-(3-(6-azaspiro[2.5]octan-6-yl)-4-(4- (spiro[benzo[d][1,3]dioxole-2,1′- cyclohexan]-4-yl)-1H-1,2,3-triazol-1- yl)phenyl)methanesulfonamide |
| Compound 129 | 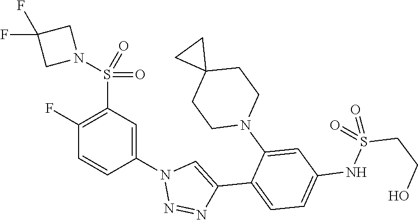 [SMILES: O=S(=O)(CCO)Nc1ccc(-c2cn(-c3ccc(F)c(S(=O)(=O)N4CC(F)(F)C4)c3)nn2)c(N2CCC3(CC2)CC3)c1] | N-(4-(1-(3-((3,3-difluoroazetidin-1- yl)sulfonyl)-4-fluorophenyl)-1H-1,2,3- triazol-4-yl)-3-(6-azaspiro[2.5]octan-6- yl)phenyl)-2-hydroxyethane-1- sulfonamide |
| Compound 130 | 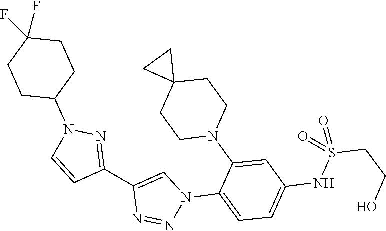 [SMILES: O=S(=O)(CCO)Nc1ccc(-n2cc(-c3ccn(C4CCC(F)(F)CC4)n3)nn2)c(N2CCC3(CC2)CC3)c1] | N-(4-(4-(1-(4,4-difluorocyclohexyl)-1H- pyrazol-3-yl)-1H-1,2,3-triazol-1-yl)-3-(7- azaspiro[3.5]nonan-7-yl)phenyl)-2- hydroxyethane-1-sulfonamide |
| Compound 131 | 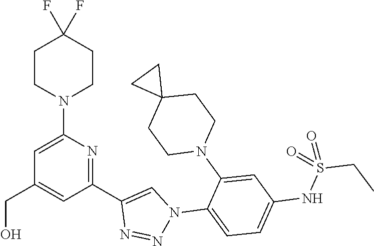 [SMILES: CCS(=O)(=O)Nc1ccc(-n2cc(-c3cc(CO)cc(N4CCC(F)(F)CC4)n3)nn2)c(N2CCC3(CC2)CC3)c1] | N-(4-(4-(6-(4,4-difluoropiperidin-1-yl)-4- (hydroxymethyl)pyridin-2-yl)-1H-1,2,3- triazol-1-yl)-3-(6-azaspiro[2.5]octan-6- yl)phenyl)ethanesulfonamide |
| Compound 132 | 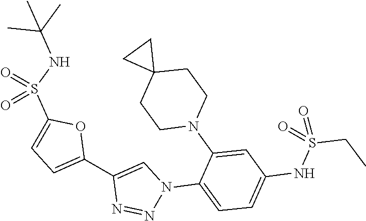 [SMILES: CCS(=O)(=O)Nc1ccc(-n2cc(-c3ccc(S(=O)(=O)NC(C)(C)C)o3)nn2)c(N2CCC3(CC2)CC3)c1] | N-(tert-butyl)-5-(1-(4-(ethylsulfonamido)- 2-(6-azaspiro[2.5]octan-6-yl)phenyl)-1H- 1,2,3-triazol-4-yl)furan-2-sulfonamide |
| Compound 133 | 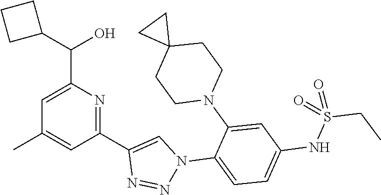 [SMILES: CCS(=O)(=O)Nc1ccc(-n2cc(-c3cc(C)cc(C(O)C4CCC4)n3)nn2)c(N2CCC3(CC2)CC3)c1] | N-(4-(4-(6-(cyclobutyl(hydroxy)methyl)- 4-methylpyridin-2-yl)-1H-1,2,3-triazol-1- yl)-3-(6-azaspiro[2.5]octan-6- yl)phenyl)ethanesulfonamide |
| Compound 134 | 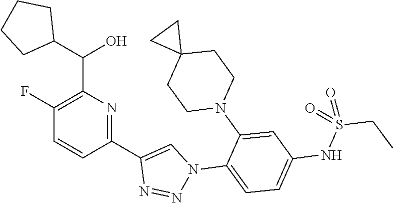 [SMILES: CCS(=O)(=O)Nc1ccc(-n2cc(-c3ccc(F)c(C(O)C4CCCC4)n3)nn2)c(N2CCC3(CC2)CC3)c1] | N-(4-(4-(6-(cyclopentyl(hydroxy)methyl)- 5-fluoropyridin-2-yl)-1H-1,2,3-triazol-1- yl)-3-(6-azaspiro[2.5]octan-6- yl)phenyl)ethanesulfonamide |
| Compound 135 | 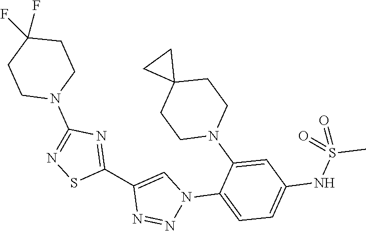 [SMILES: CS(=O)(=O)Nc1ccc(-n2cc(-c3nc(N4CCC(F)(F)CC4)ns3)nn2)c(N2CCC3(CC2)CC3)c1] | N-(4-(4-(3-(4,4-difluoropiperidin-1-yl)- 1,2,4-thiadiazol-5-yl)-1H-1,2,3-triazol-1- yl)-3-(6-azaspiro[2.5]octan-6- yl)phenyl)methanesulfonamide |
| Compound 136 | 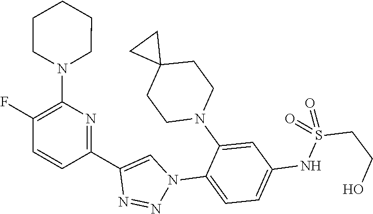 [SMILES: O=S(=O)(CCO)Nc1ccc(-n2cc(-c3ccc(F)c(N4CCCCC4)n3)nn2)c(N2CCC3(CC2)CC3)c1] | N-(4-(4-(5-fluoro-6-(piperidin-1- yl)pyridin-2-yl)-1H-1,2,3-triazol-1-yl)-3- (6-azaspiro[2.5]octan-6-yl)phenyl)-2- hydroxyethane-1-sulfonamide |
| Compound 137 | 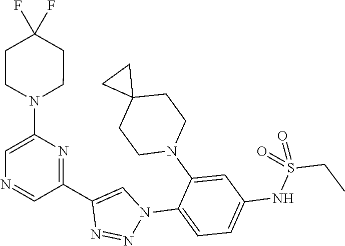 [SMILES: CCS(=O)(=O)Nc1ccc(-n2cc(-c3cncc(N4CCC(F)(F)CC4)n3)nn2)c(N2CCC3(CC2)CC3)c1] | N-(4-(4-(6-(4,4-difluoropiperidin-1- yl)pyrazin-2-yl)-1H-1,2,3-triazol-1-yl)-3- (6-azaspiro[2.5]octan-6- yl)phenyl)ethanesulfonamide |
| Compound 138 | 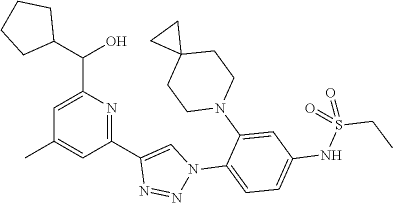 [SMILES: CCS(=O)(=O)Nc1ccc(-n2cc(-c3cc(C)cc(C(O)C4CCCC4)n3)nn2)c(N2CCC3(CC2)CC3)c1] | N-(4-(4-(6-(cyclopentyl(hydroxy)methyl)- 4-methylpyridin-2-yl)-1H-1,2,3-triazol-1- yl)-3-(6-azaspiro[2.5]octan-6- yl)phenyl)ethanesulfonamide |
| Compound 139 | 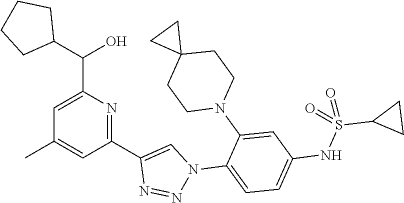 [SMILES: Cc1cc(-c2cn(-c3ccc(NS(=O)(=O)C4CC4)cc3N3CCC4(CC3)CC4)nn2)nc(C(O)C2CCCC2)c1] | N-(4-(4-(6-(cyclopentyl(hydroxy)methyl)- 4-methylpyridin-2-yl)-1H-1,2,3-triazol-1- yl)-3-(6-azaspiro[2.5]octan-6- yl)phenyl)cyclopropanesulfonamide |
| Compound 140 | 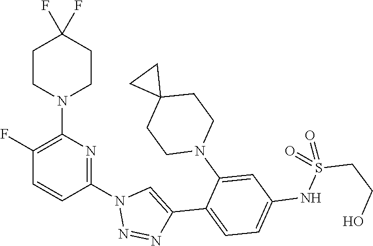 [SMILES: O=S(=O)(CCO)Nc1ccc(-c2cn(-c3ccc(F)c(N4CCC(F)(F)CC4)n3)nn2)c(N2CCC3(CC2)CC3)c1] | N-(4-(1-(6-(4,4-difluoropiperidin-1-yl)-5- fluoropyridin-2-yl)-1H-1,2,3-triazol-4-yl)- 3-(6-azaspiro[2.5]octan-6-yl)phenyl)-2- hydroxyethane-1-sulfonamide |
| Compound 141 | 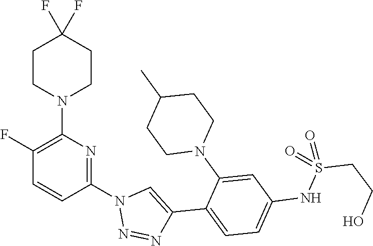 [SMILES: CC1CCN(c2cc(NS(=O)(=O)CCO)ccc2-c2cn(-c3ccc(F)c(N4CCC(F)(F)CC4)n3)nn2)CC1] | N-(4-(1-(6-(4,4-difluoropiperidin-1-yl)-5- fluoropyridin-2-yl)-1H-1,2,3-triazol-4-yl)- 3-(4-methylpiperidin-1-yl)phenyl)-2- hydroxyethane-1-sulfonamide |
| Compound 142 | 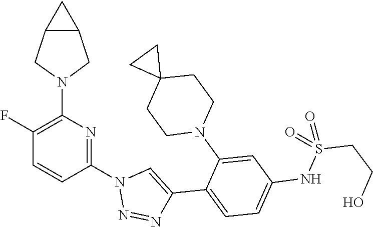 [SMILES: O=S(=O)(CCO)Nc1ccc(-c2cn(-c3ccc(F)c(N4CC5CC5C4)n3)nn2)c(N2CCC3(CC2)CC3)c1] | N-(4-(1-(6-(3-azabicyclo[3.1.0]hexan-3- yl)-5-fluoropyridin-2-yl)-1H-1,2,3-triazol- 4-yl)-3-(6-azaspiro[2.5]octan-6- yl)phenyl)-2-hydroxyethane-1- sulfonamide |
| Compound 143 | 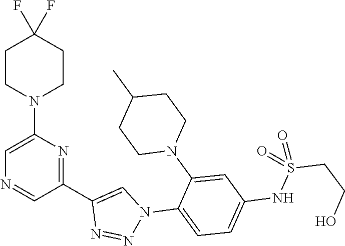 [SMILES: CC1CCN(c2cc(NS(=O)(=O)CCO)ccc2-n2cc(-c3cncc(N4CCC(F)(F)CC4)n3)nn2)CC1] | N-(4-(4-(6-(4,4-difluoropiperidin-1- yl)pyrazin-2-yl)-1H-1,2,3-triazol-1-yl)-3- (4-methylpiperidin-1-yl)phenyl)-2- hydroxyethane-1-sulfonamide |
| Compound 144 | 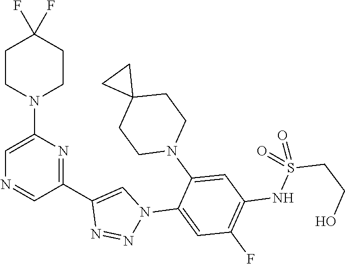 [SMILES: O=S(=O)(CCO)Nc1cc(N2CCC3(CC2)CC3)c(-n2cc(-c3cncc(N4CCC(F)(F)CC4)n3)nn2)cc1F] | N-(4-(4-(6-(4,4-difluoropiperidin-1- yl)pyrazin-2-yl)-1H-1,2,3-triazol-1-yl)-2- fluoro-5-(6-azaspiro[2.5]octan-6- yl)phenyl)-2-hydroxyethane-1- sulfonamide |
| Compound 145 | 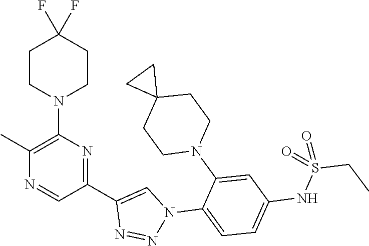 [SMILES: CCS(=O)(=O)Nc1ccc(-n2cc(-c3cnc(C)c(N4CCC(F)(F)CC4)n3)nn2)c(N2CCC3(CC2)CC3)c1] | N-(4-(4-(6-(4,4-difluoropiperidin-1-yl)-5- methylpyrazin-2-yl)-1H-1,2,3-triazol-1- yl)-3-(6-azaspiro[2.5]octan-6- yl)phenyl)ethanesulfonamide |
| Compound 146 | 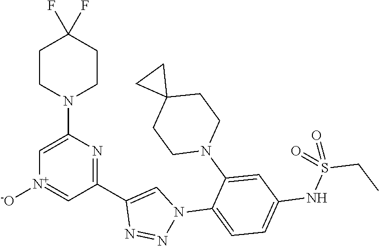 | 3-(4,4-difluoropiperidin-1-yl)-5-(1-(4- (ethylsulfonamido)-2-(6- azaspiro[2.5]octan-6-yl)phenyl)-1H-1,2,3- triazol-4-yl)pyrazine 1-oxide |
| Compound 147 | 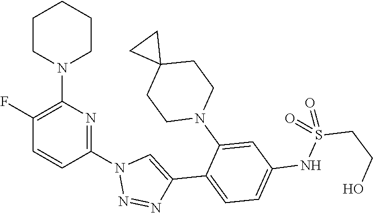 [SMILES: O=S(=O)(CCO)Nc1ccc(-c2cn(-c3ccc(F)c(N4CCCCC4)n3)nn2)c(N2CCC3(CC2)CC3)c1] | N-(4-(1-(5-fluoro-6-(piperidin-1- yl)pyridin-2-yl)-1H-1,2,3-triazol-4-yl)-3- (6-azaspiro[2.5]octan-6-yl)phenyl)-2- hydroxyethane-1-sulfonamide |
| Compound 148 | 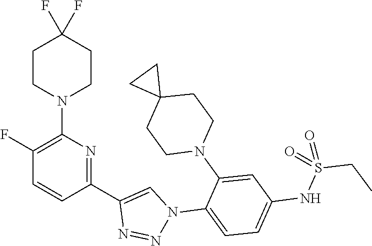 [SMILES: CCS(=O)(=O)Nc1ccc(-n2cc(-c3ccc(F)c(N4CCC(F)(F)CC4)n3)nn2)c(N2CCC3(CC2)CC3)c1] | N-(4-(4-(6-(4,4-difluoropiperidin-1-yl)-5- fluoropyridin-2-yl)-1H-1,2,3-triazol-1-yl)- 3-(6-azaspiro[2.5]octan-6- yl)phenyl)ethanesulfonamide |
| Compound 149 | 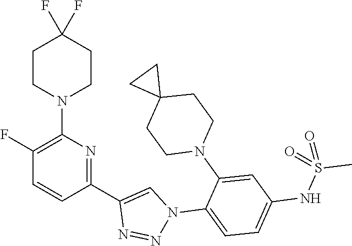 [SMILES: CS(=O)(=O)Nc1ccc(-n2cc(-c3ccc(F)c(N4CCC(F)(F)CC4)n3)nn2)c(N2CCC3(CC2)CC3)c1] | N-(4-(4-(6-(4,4-difluoropiperidin-1-yl)-5- fluoropyridin-2-yl)-1H-1,2,3-triazol-1-yl)- 3-(6-azaspiro[2.5]octan-6- yl)phenyl)methanesulfonamide |
| Compound 150 | 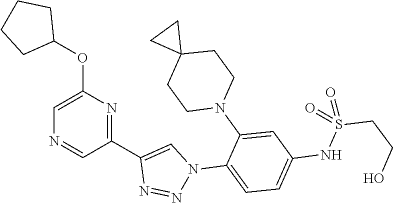 [SMILES: O=S(=O)(CCO)Nc1ccc(-n2cc(-c3cncc(OC4CCCC4)n3)nn2)c(N2CCC3(CC2)CC3)c1] | N-(4-(4-(6-(cyclpentyloxy)pyrazin-2-yl)- 1H-1,2,3-triazol-1-yl)-3-(6- azaspiro[2.5]octan-6-yl)phenyl)-2- hydroxyethane-1-sulfonamide |
| Compound 151 | 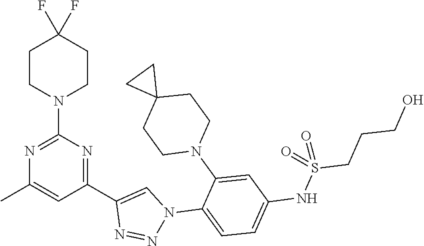 [SMILES: Cc1cc(-c2cn(-c3ccc(NS(=O)(=O)CCCO)cc3N3CCC4(CC3)CC4)nn2)nc(N2CCC(F)(F)CC2)n1] | N-(4-(4-(2-(4,4-difluoropiperidin-1-yl)-6- methylpyrimidin-4-yl)-1H-1,2,3-triazol-1- yl)-3-(6-azaspiro[2.5]octan-6-yl)phenyl)- 3-hydroxypropane-1-sulfonamide |
| Compound 152 | 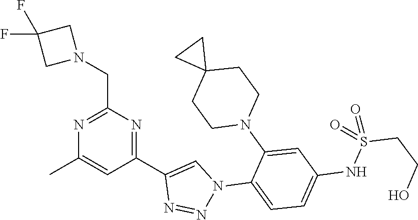 [SMILES: Cc1cc(-c2cn(-c3ccc(NS(=O)(=O)CCO)cc3N3CCC4(CC3)CC4)nn2)nc(CN2CC(F)(F)C2)n1] | N-(4-(4-(2-((3,3-difluoroazetidin-1- yl)methyl)-6-methylpyrimidin-4-yl)-1H- 1,2,3-triazol-1-yl)-3-(6-azaspiro[2.5]octan- 6-yl)phenyl)-2-hydroxyethane-1- sulfonamide |
| Compound 153 | 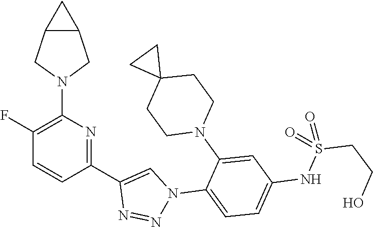 [SMILES: O=S(=O)(CCO)Nc1ccc(-n2cc(-c3ccc(F)c(N4CC5CC5C4)n3)nn2)c(N2CCC3(CC2)CC3)c1] | N-(4-(4-(6-(3-azabicyclo[3.1.0]hexan-3- yl)-5-fluoropyridin-2-yl)-1H-1,2,3-triazol- 1-yl)-3-(6-azaspiro[2.5]octan-6- yl)phenyl)-2-hydroxyethane-1- sulfonamide |
| Compound 154 | 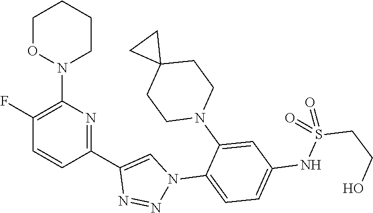 [SMILES: O=S(=O)(CCO)Nc1ccc(-n2cc(-c3ccc(F)c(N4CCCCO4)n3)nn2)c(N2CCC3(CC2)CC3)c1] | N-(4-(4-(5-fluoro-6-(1,2-oxazinan-2- yl)pyridin-2-yl)-1H-1,2,3-triazol-1-yl)-3- (6-azaspiro[2.5]octan-6-yl)phenyl)-2- hydroxyethane-1-sulfonamide |
| Compound 155 | 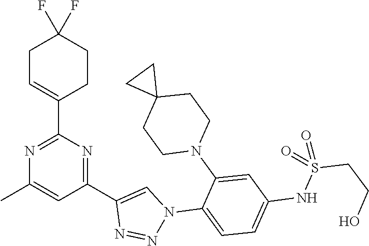 [SMILES: Cc1cc(-c2cn(-c3ccc(NS(=O)(=O)CCO)cc3N3CCC4(CC3)CC4)nn2)nc(C2=CCC(F)(F)CC2)n1] | N-(4-(4-(2-(4,4-difluorocyclohex-1-en-1- yl)-6-methylpyrimidin-4-yl)-1H-1,2,3- triazol-1-yl)-3-(6-azaspiro[2.5]octan-6- yl)phenyl)-2-hydroxyethane-1- sulfonamide |
| Compound 156 | 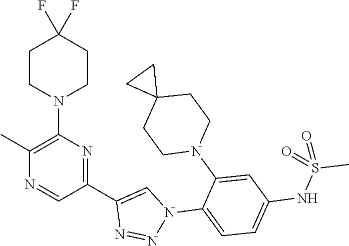 | N-(4-(4-(6-(4,4-difluoropiperidin-1-yl)-5- methylpyrazin-2-yl)-1H-1,2,3-triazol-1- yl)-3-(6-azaspiro[2.5]octan-6- yl)phenyl)methanesulfonamide |
| Compound 157 | 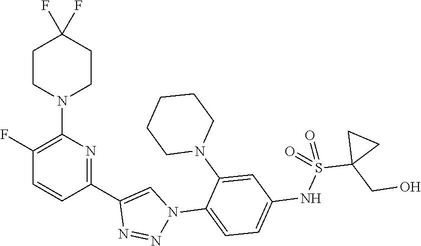 [SMILES: O=S(=O)(Nc1ccc(-n2cc(-c3ccc(F)c(N4CCC(F)(F)CC4)n3)nn2)c(N2CCCCC2)c1)C1(CO)CC1] | N-(4-(4-(6-(4,4-difluoropiperidin-1-yl)-5- fluoropyridin-2-yl)-1H-1,2,3-triazol-1-yl)- 3-(piperidin-1-yl)phenyl)-1- (hydroxymethyl)cyclopropane-1- sulfonamide |
| Compound 158 | 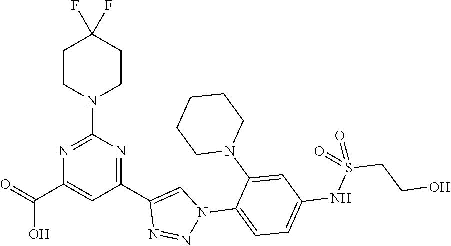 [SMILES: O=C(O)c1cc(-c2cn(-c3ccc(NS(=O)(=O)CCO)cc3N3CCCCC3)nn2)nc(N2CCC(F)(F)CC2)n1] | 2-(4,4-difluoropiperidin-1-yl)-6-(1-(4-((2- hydroxyethyl)sulfonamido)-2-(6- azaspiro[2.5]octan-6-yl)phenyl)-1H-1,2,3- triazol-4-yl)pyrimidine-4-carboxylic acid |
| Compound 159 | 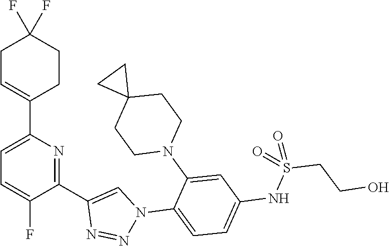 [SMILES: O=S(=O)(CCO)Nc1ccc(-n2cc(-c3nc(C4=CCC(F)(F)CC4)ccc3F)nn2)c(N2CCC3(CC2)CC3)c1] | N-(4-(4-(6-(4,4-difluorocyclohex-1-en-1- yl)-3-fluoropyridin-2-yl)-1H-1,2,3-triazol- 1-yl)-3-(6-azaspiro[2.5]octan-6- yl)phenyl)-2-hydroxyethane-1- sulfonamide |
| Compound 160 | 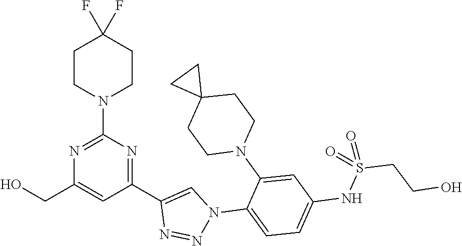 [SMILES: O=S(=O)(CCO)Nc1ccc(-n2cc(-c3cc(CO)nc(N4CCC(F)(F)CC4)n3)nn2)c(N2CCC3(CC2)CC3)c1] | N-(4-(4-(2-(4,4-difluoropiperidin-1-yl)-6- (hydroxymethyl)pyrimidin-4-yl)-1H-1,2,3- triazol-1-yl)-3-(6-azaspiro[2.5]octan-6- yl)phenyl)-2-hydroxyethane-1- sulfonamide |
| Compound 161 | 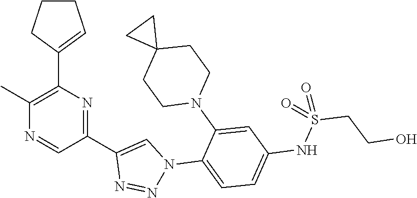 [SMILES: Cc1ncc(-c2cn(-c3ccc(NS(=O)(=O)CCO)cc3N3CCC4(CC3)CC4)nn2)nc1C1=CCCC1] | N-(4-(4-(6-(cyclopent-1-en-1-yl)-5- methylpyrazin-2-yl)-1H-1,2,3-triazol-1- yl)-3-(6-azaspiro[2.5]octan-6-yl)phenyl)- 2-hydroxyethane-1-sulfonamide |
| Compound 162 | 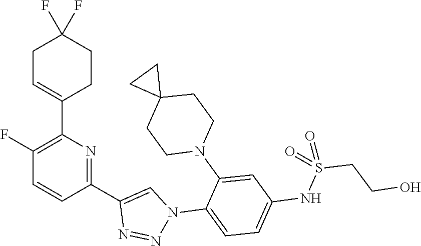 [SMILES: O=S(=O)(CCO)Nc1ccc(-n2cc(-c3ccc(F)c(C4=CCC(F)(F)CC4)n3)nn2)c(N2CCC3(CC2)CC3)c1] | N-(4-(4-(6-(4,4-difluorocyclohex-1-en-1- yl)-5-fluoropyridin-2-yl)-1H-1,2,3-triazol- 1-yl)-3-(6-azaspiro[2.5]octan-6- yl)phenyl)-2-hydroxyethane-1- sulfonamide |
| Compound 163 | 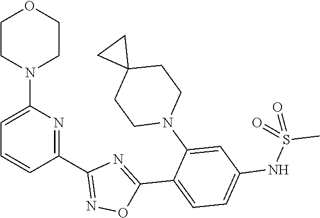 [SMILES: CS(=O)(=O)Nc1ccc(-c2nc(-c3cccc(N4CCOCC4)n3)no2)c(N2CCC3(CC2)CC3)c1] | N-(4-(3-(6-morpholinopyridin-2-yl)-1,2,4- oxadiazol-5-yl)-3-(6-azaspiro[2.5]octan-6- yl)phenyl)methanesulfonamide |
| Compound 164 | 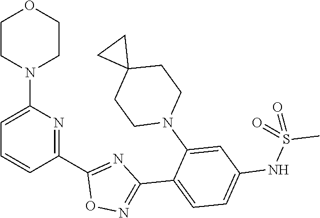 [SMILES: CS(=O)(=O)Nc1ccc(-c2noc(-c3cccc(N4CCOCC4)n3)n2)c(N2CCC3(CC2)CC3)c1] | N-(4-(5-(6-morpholinopyridin-2-yl)-1,2,4- oxadiazol-3-yl)-3-(6-azaspiro[2.5]octan-6- yl)phenyl)methanesulfonamide |
| Compound 165 | 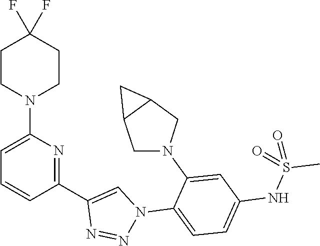 [SMILES: CS(=O)(=O)Nc1ccc(-n2cc(-c3cccc(N4CCC(F)(F)CC4)n3)nn2)c(N2CC3CC3C2)c1] | N-(3-(3-azabicyclo[3.1.0]hexan-3-yl)-4- (4-(6-(4,4-difluoropiperidin-1-yl)pyridin- 2-yl)-1H-1,2,3-triazol-1- yl)phenyl)methanesulfonamide |
| Compound 166 | 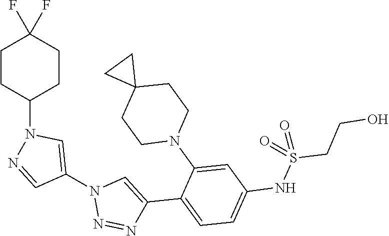 [SMILES: O=S(=O)(CCO)Nc1ccc(-c2cn(-c3cnn(C4CCC(F)(F)CC4)c3)nn2)c(N2CCC3(CC2)CC3)c1] | N-(4-(1-(1-(4,4-difluorocyclohexyl)-1H- pyrazol-4-yl)-1H-1,2,3-triazol-4-yl)-3-(6- azaspiro[2.5]octan-6-yl)phenyl)-2- hydroxyethane-1-sulfonamide |

In some variations, any of the compounds described herein, such as a compound of Formula (I), Formula (II), Formula (III), or any variation thereof, or a compound of Table 1 may be deuterated (e.g., a hydrogen atom is replaced by a deuterium atom). In some of these variations, the compound is deuterated at a single site. In other variations, the compound is deuterated at multiple sites. Deuterated compounds can be prepared from deuterated starting materials in a manner similar to the preparation of the corresponding non-deuterated compounds. Hydrogen atoms may also be replaced with deuterium atoms using other method known in the art.

Any formula given herein, such as Formula (I), Formula (II), or Formula (III), is intended to represent compounds having structures depicted by the structural formula as well as certain variations or forms. In particular, compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio. Furthermore, certain structures may exist as geometric isomers (i.e., cis and trans isomers), as tautomers, or as atropisomers. Additionally, any formula given herein is intended to refer also to any one of hydrates, solvates, and amorphous and polymorphic forms of such compounds, and mixtures thereof, even if such forms are not listed explicitly. In some embodiments, the solvent is water and the solvates are hydrates.

Representative examples of compounds detailed herein, including intermediates and final compounds, are depicted in the tables and elsewhere herein. It is understood that in one aspect, any of the compounds may be used in the methods detailed herein, including, where applicable, intermediate compounds that may be isolated and administered to an individual.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

In one variation, the compounds herein are synthetic compounds prepared for administration to an individual. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of V, W, X, Y, Z, Ring A, A¹, A², A³, A⁴, A⁵, A⁶, A⁷, A⁸, R^{A1}, R^{A2}, B¹, B², R, R¹, R², R³, R⁴, R^{a1}, R^{a2}, R^{a3}, R^{a4}, R^{a5}, R^{a6}, R^{a7}, R^{a8}, R^{a9}, R^{a10}, R^{a11}, R^{a12}, R^{a13}, R^{a14}, R^{a15}, R^{a16}, R^{a17}, R^{a18}, R^{a19}, R^{a20}, R^{1a1}, R^{1a2}, R^{b1}, R^{b2}, R^{c1}, R^{c2}, R^{c3}, R^{c4}, R^{c5}, R^{c6}, R^{c7}, R^{c8}, R^{c9}, R^{c10}, R^{c11}, R^{c12}, R^{c13}, or R^{d1}, provided herein can be combined with every other variation or embodiment of V, W, X, Y, Z, Ring A, A¹, A², A³, A⁴, A⁵, A⁶, A⁷, A⁸, R^{A1}, R^{A2}, B¹, B², R^{B}, R¹, R², R³, R⁴, R^{a1}, R^{a2}, R^{a3}, R^{a4}, R^{a5}, R^{a6}, R^{a7}, R^{a8}, R^{a9}, R^{a10}, R^{a11}, R^{a2}, R^{a3}, R^{a14}, R^{a15}, R^{a16}, R^{a17}, R^{a18}, R^{a19}, R^{a20}, R^{1a1}, R^{1a2}, R^{b1}, R^{b2}, R^{c1}, R^{c2}, R^{c3}, R^{c4}, R^{c5}, R^{c6}, R^{c7}, R^{c8}, R^{c9}, R^{c10}, R^{c11}, R^{c12}, R^{c13}, or R^{d1}, as if each combination had been individually and specifically described.

As used herein, when any variable occurs more than one time in a chemical formula, its definition on each occurrence is independent of its definition at every other occurrence.

Compound names provided herein, including in Table 1 are provided by Chemaxon Marvin Structure to Name 20 or ChemDraw Professional 21. One of skilled in the art would understand that the compounds may be named or identified using various commonly recognized nomenclature systems and symbols. By way of example, the compounds may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

### Compositions

Also provided are compositions, such as pharmaceutical compositions, that include a compound disclosed and/or described herein and one or more additional medicinal agents, pharmaceutical agents, adjuvants, carriers, excipients, and the like. Suitable medicinal and pharmaceutical agents include those described herein. In some embodiments, the pharmaceutical composition includes a pharmaceutically acceptable excipient or adjuvant and at least one chemical entity as described herein. Examples of pharmaceutically acceptable excipients include, but are not limited to, mannitol, lactose, starch, magnesium stearate, sodium saccharine, talcum, cellulose, sodium crosscarmellose, glucose, gelatin, sucrose, and magnesium carbonate. In some embodiments, provided are compositions, such as pharmaceutical compositions that contain one or more compounds described herein, or a pharmaceutically acceptable salt thereof.

In some embodiments, provided is a pharmaceutically acceptable composition comprising a compound of Formula (I), Formula (II), Formula (III), or a compound of Table 1, or a pharmaceutically acceptable salt thereof. In some aspects, a composition may contain a synthetic intermediate that may be used in the preparation of a compound described herein. The compositions described herein may contain any other suitable active or inactive agents.

Any of the compositions described herein may be sterile or contain components that are sterile. Sterilization can be achieved by methods known in the art. Any of the compositions described herein may contain one or more compounds that are substantially pure.

Also provided are packaged pharmaceutical compositions, comprising a pharmaceutical composition as described herein and instructions for using the composition to treat a patient suffering from a disease or condition described herein.

### Methods of Use

As described herein, the compounds of the present disclosure are inhibitors of KIF18A. In one aspect, the compounds and pharmaceutical compositions herein may be used to inhibit KIF18A. In another aspect, the compounds and pharmaceutical compositions herein may be used to treat or prevent a disease or condition in an individual.

The inhibitory activity of the compounds described herein against KIF18A may be determined and measured by methods known in the art including, but not limited to, inhibition of ATP hydrolysis in the presence of microtubules (Hackney D. D., Jiang W. (2001) Assays for Kinesin Microtubule-Stimulated ATPase Activity. In: Vernos I. (eds) Kinesin Protocols. Methods in Molecular Biology™, vol 164. Humana Press. https://doi.org/10.1385/1-59259-069-1:65).

In one aspect, provided herein is a method of inhibiting KIF18A comprising contacting a cell with an effective amount of a compound or a pharmaceutical composition as described herein. In some embodiments, provided herein are methods of inhibiting KIF18A comprising contacting a cell with an effective amount of a compound of Formula (I), Formula (II), Formula (III), or a compound of Table 1, or a pharmaceutically acceptable salt thereof. In some embodiments, provided herein are methods of inhibiting KIF18A comprising contacting a cell with an effective amount of a pharmaceutical composition comprising a compound of Formula (I), Formula (II), Formula (III), or a compound of Table 1, or a pharmaceutically acceptable salt thereof. In one variations of the aforementioned embodiments, the cell is contacted in vitro. In other variations of the aforementioned embodiments, the cell is contacted in vivo.

In another aspect, the compounds and pharmaceutical compositions herein may be used to treat or prevent a disease or condition in an individual, comprising administering an effective amount of a compound or a pharmaceutical composition as described herein. When used in a prophylactic manner, the compounds disclosed and/or described herein may prevent a disease or disorder from developing in an individual at risk of developing the disease or disorder, or lessen the extent of a disease or disorder that may develop.

In some embodiments, provided herein are methods of treating or preventing a disease or condition in an individual, comprising administering to the subject a therapeutically effective amount of a compound or a pharmaceutical composition as described herein. In some embodiments, provided herein are methods of treating or preventing a disease or condition in an individual, comprising administering to the subject a therapeutically effective amount of a compound of Formula (I), Formula (II), Formula (III), or a compound of Table 1, or a pharmaceutically acceptable salt thereof. In some embodiments, provided herein are methods of treating or preventing a disease or condition in an individual, comprising administering to the subject a therapeutically effective amount of a pharmaceutical composition comprising a compound a compound of Formula (I), Formula (II), Formula (III), or a compound of Table 1, or a pharmaceutically acceptable salt thereof.

In some embodiments, the disease or condition is mediated by KIF18A. In some embodiments, the disease or condition is cancer. In some embodiments, the disease or condition is a cellular proliferation disorder, including uncontrolled cell growth, aberrant cell cycle regulation, centrosome abnormalities (structural and or numeric, fragmentation), a solid tumor, hematopoietic cancer and hyperproliferative disorder, such as thyroid hyperplasia (especially Grave's disease), and cyst (such as hypervascularity of ovarian stroma, characteristic of polycystic ovarian syndrome (Stein-Leventhal syndrome). Solid and hematologically derived tumors, such as carcinomas, may include but are not limited to cancer of the anus, bladder, breast, colon, small intestine, appendix, kidney, renal pelvis, ureter, urothelium, liver, lung (including squamous cell and small cell lung cancer), pleura, esophagus, head and neck, nasopharynx, oropharynx, hypopharynx, oral cavity, larynx, biliary tract, gall-bladder, ovary, testicle, germ cell, uterus, pancreas, stomach, cervix, thyroid, prostate, salivary gland, and skin (including squamous cell carcinoma), hematopoietic tumors of lymphoid lineage (including leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma), hematopoietic tumors of myeloid lineage (including acute and chronic myelogenous leukemias, myelodysplastic syndrome and promyelocytic leukemia), hematopoietic tumors of any lineage, myeloma, tumors of mesenchymal origin (including fibrosarcoma and rhabdomyosarcoma, and other sarcomas, e.g., soft tissue and bone), tumors of the central and peripheral nervous system (including astrocytoma, neuroblastoma, glioma and schwannomas), tumor of neuroendocrine origin, tumor of endocrine origin, small cell tumors, tumors of unknown primary, other tumors (including retinoblastoma, melanoma, seminoma, teratocarcinoma, osteosarcoma, xeroderma pigmentosum, keratoacanthoma, thyroid follicular cancer, Ewing's sarcoma, Kaposi's sarcoma), and other cancer-related disorders that are a consequence of cancer presence or progression such as tumor-induced pleural or pericardial effusions, and malignant ascites.

In some embodiments, provided are methods of treating or preventing cancer in an individual, comprising administering to the individual in need thereof a compound of Formula (I), Formula (II), Formula (III), or a compound of Table 1, or a pharmaceutically acceptable salt thereof. In some embodiments, provided are methods of treating or preventing cancer in a subject in need thereof comprising administering to the subject a therapeutically effective amount of at least one chemical entity as described herein. Also provided herein is the use of a compound of Formula (I), Formula (TI), Formula (III), or a compound of Table 1, or a pharmaceutically acceptable salt thereof in the manufacture of a medicament for treatment of a disease in a subject.

In some embodiments, provided herein are methods of treating cancer, comprising administering to an individual in need thereof a compound of Formula (I), Formula (II), Formula (III), or a compound of Table 1, or a pharmaceutically acceptable salt thereof. Also provided herein is the use of a compound of Formula (I), Formula (II), Formula (III), or a compound of Table 1, or a pharmaceutically acceptable salt thereof in the manufacture of a medicament for treatment of a cancer.

In some embodiments, provided herein are methods of treating a disease or condition mediated by KIF18A in a subject in need thereof, comprising administering to the subject a therapeutically effective amount of a compound or a pharmaceutical composition as described herein.

In some embodiments, provided herein are methods of treating cancer in a subject in need thereof, comprising administering to the subject a therapeutically effective amount of a compound or a pharmaceutical composition as described herein. In some embodiments, the cancer is selected from the group consisting of carcinomas, cancer of the anus, bladder, breast, colon, small intestine, appendix, kidney, renal pelvis, ureter, urothelium, liver, lung, pleura, esophagus, head and neck, nasopharynx, oropharynx, hypopharynx, oral cavity, larynx, biliary tract, gall-bladder, ovary, testicle, germ cell, uterus, pancreas, stomach, cervix, thyroid, prostate, salivary gland, or skin, hematopoietic tumors of lymphoid lineage, hematopoietic tumors of myeloid lineage, hematopoietic tumors of any lineage, myeloma, tumors of mesenchymal origin including sarcomas, tumors of the central and peripheral nervous system, tumor of neuroendocrine origin, tumor of endocrine origin, small cell tumors, tumors of unknown primary, other tumors comprising retinoblastoma, melanoma, seminoma, teratocarcinoma, osteosarcoma, and other cancer-related disorders that are a consequence of cancer presence or progression.

### Dosages

The compounds and compositions disclosed and/or described herein are administered at a therapeutically effective dosage, e.g., a dosage sufficient to provide treatment for the disease state. While human dosage levels have yet to be optimized for the chemical entities described herein, generally, a daily dose ranges from about 0.01 to 100 mg/kg of body weight; in some embodiments, from about 0.05 to 10.0 mg/kg of body weight, and in some embodiments, from about 0.10 to 1.4 mg/kg of body weight. Thus, for administration to a 70 kg person, in some embodiments, the dosage range would be about from 0.7 to 7000 mg per day; in some embodiments, about from 3.5 to 700.0 mg per day, and in some embodiments, about from 7 to 100.0 mg per day. The amount of the chemical entity administered will be dependent, for example, on the subject and disease state being treated, the severity of the affliction, the manner and schedule of administration and the judgment of the prescribing physician. For example, an exemplary dosage range for oral administration is from about 5 mg to about 500 mg per day, and an exemplary intravenous administration dosage is from about 5 mg to about 500 mg per day, each depending upon the compound pharmacokinetics.

Administration of the compounds and compositions disclosed and/or described herein can be via any accepted mode of administration for therapeutic agents including, but not limited to, oral, sublingual, subcutaneous, parenteral, intravenous, intranasal, topical, transdermal, intraperitoneal, intramuscular, intrapulmonary, vaginal, rectal, or intraocular administration. In some embodiments, the compound or composition is administered orally or intravenously. In some embodiments, the compound or composition disclosed and/or described herein is administered orally.

Pharmaceutically acceptable compositions include solid, semi-solid, liquid and aerosol dosage forms, such as tablet, capsule, powder, liquid, suspension, suppository, and aerosol forms. The compounds disclosed and/or described herein can also be administered in sustained or controlled release dosage forms (e.g., controlled/sustained release pill, depot injection, osmotic pump, or transdermal (including electrotransport) patch forms) for prolonged timed, and/or pulsed administration at a predetermined rate. In some embodiments, the compositions are provided in unit dosage forms suitable for single administration of a precise dose.

The compounds disclosed and/or described herein can be administered either alone or in combination with one or more conventional pharmaceutical carriers or excipients (e.g., mannitol, lactose, starch, magnesium stearate, sodium saccharine, talcum, cellulose, sodium crosscarmellose, glucose, gelatin, sucrose, magnesium carbonate). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate). Generally, depending on the intended mode of administration, the pharmaceutical composition will contain about 0.005% to 95%, or about 0.5% to 50%, by weight of a compound disclosed and/or described herein. Actual methods of preparing such dosage forms are known, or will be apparent, to those skilled in this art; for example, see Remington's Pharmaceutical Sciences, Mack Publishing Company, Easton, Pennsylvania.

In some embodiments, the compositions will take the form of a pill or tablet and thus the composition may contain, along with a compounds disclosed and/or described herein, one or more of a diluent (e.g., lactose, sucrose, dicalcium phosphate), a lubricant (e.g., magnesium stearate), and/or a binder (e.g., starch, gum acacia, polyvinylpyrrolidine, gelatin, cellulose, cellulose derivatives). Other solid dosage forms include a powder, marume, solution or suspension (e.g., in propylene carbonate, vegetable oils or triglycerides) encapsulated in a gelatin capsule.

Liquid pharmaceutically administrable compositions can, for example, be prepared by dissolving, dispersing or suspending etc. a compound disclosed and/or described herein and optional pharmaceutical additives in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of the compound contained in such parenteral compositions depends, for example, on the physical nature of the compound, the activity of the compound and the needs of the subject. However, percentages of active ingredient of 0.01% to 10% in solution are employable, and may be higher if the composition is a solid which will be subsequently diluted to another concentration. In some embodiments, the composition will comprise from about 0.2 to 2% of a compound disclosed and/or described herein in solution.

Pharmaceutical compositions of the compounds disclosed and/or described herein may also be administered to the respiratory tract as an aerosol or solution for a nebulizer, or as a microfine powder for insufflation, alone or in combination with an inert carrier such as lactose. In such a case, the particles of the pharmaceutical composition may have diameters of less than 50 microns, or in some embodiments, less than 10 microns.

In addition, pharmaceutical compositions can include a compound disclosed and/or described herein and one or more additional medicinal agents, pharmaceutical agents, adjuvants, and the like. Suitable medicinal and pharmaceutical agents include those described herein.

### Kits

Also provided are articles of manufacture and kits containing any of the compounds or pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing a compound or composition described herein and instructions for use. The kits may contain instructions for use in the treatment of any disease or condition described herein in an individual in need thereof. A kit may additionally contain any materials or equipment that may be used in the administration of the compound or composition, such as vials, syringes, or IV bags. A kit may also contain sterile packaging.

### Combinations

The compounds and compositions described and/or disclosed herein may be administered alone or in combination with other therapies and/or therapeutic agents useful in the treatment of the aforementioned disorders.

The compounds and compositions described and/or disclosed herein may be combined with one or more other therapies to treat the diseases or conditions described herein. In some embodiments, the disease or condition is cancer. In some embodiments, the disease or condition is a cellular proliferation disorder, including uncontrolled cell growth, aberrant cell cycle regulation, centrosome abnormalities (structural and or numeric, fragmentation), a solid tumor, hematopoietic cancer and hyperproliferative disorder, such as thyroid hyperplasia (especially Grave's disease), and cyst (such as hypervascularity of ovarian stroma, characteristic of polycystic ovarian syndrome (Stein-Leventhal syndrome). Solid and hematologically derived tumors, such as carcinomas, may include but are not limited to cancer of the anus, bladder, breast, colon, small intestine, appendix, kidney, renal pelvis, ureter, urothelium, liver, lung (including squamous cell and small cell lung cancer), pleura, esophagus, head and neck, nasopharynx, oropharynx, hypopharynx, oral cavity, larynx, biliary tract, gall-bladder, ovary, testicle, germ cell, uterus, pancreas, stomach, cervix, thyroid, prostate, salivary gland, and skin (including squamous cell carcinoma), hematopoietic tumors of lymphoid lineage (including leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma), hematopoietic tumors of myeloid lineage (including acute and chronic myelogenous leukemias, myelodysplastic syndrome and promyelocytic leukemia), hematopoietic tumors of any lineage, myeloma, tumors of mesenchymal origin (including fibrosarcoma and rhabdomyosarcoma, and other sarcomas, e.g., soft tissue and bone), tumors of the central and peripheral nervous system (including astrocytoma, neuroblastoma, glioma and schwannomas), tumor of neuroendocrine origin, tumor of endocrine origin, small cell tumors, tumors of unknown primary, other tumors (including retinoblastoma, melanoma, seminoma, teratocarcinoma, osteosarcoma, xeroderma pigmentosum, keratoacanthoma, thyroid follicular cancer, Ewing's sarcoma, Kaposi's sarcoma), and other cancer-related disorders that are a consequence of cancer presence or progression such as tumor-induced pleural or pericardial effusions, and malignant ascites.

### General Synthetic Methods

Compounds of Formula (I) will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate. Unless otherwise specified, the variables are as defined above in reference to Formula (I).

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography (HPLC). Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

General methods of preparing compounds described herein are depicted in exemplified methods below. Variable groups in the schemes provided herein are defined as for Formula (I), or any variation thereof. Other compounds described herein may be prepared by similar methods.

In some embodiments, compounds provided herein may be synthesized according to Scheme 1, Scheme 2, Scheme 3, Scheme 4, Scheme 5, Scheme 6, Scheme 7, Scheme 8, and/or Scheme 9. Ring A, A¹, A², A³, A⁴, A⁵, A⁶, A⁷, V, W, X, Y, Z, R¹, R², R³, R¹, B¹, B², R^{a1}-R^{a20}, and R^{c1}-R^{c13}, as shown in Schemes 1-9 below, are as defined for the compounds of Formula (I).

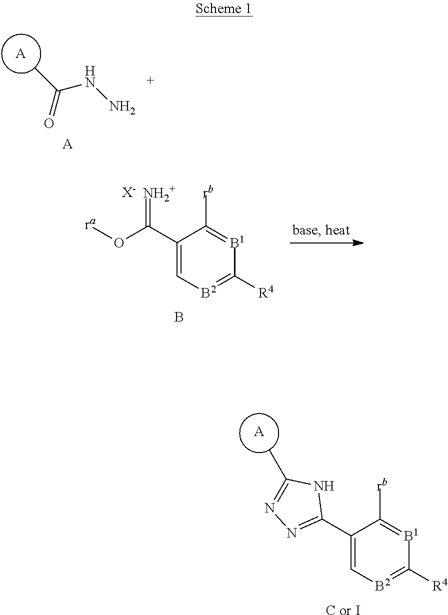

Scheme 1 outlines an exemplary route for the synthesis of compounds of Formula I when Y is “NH” and X and Z are both “N.” Acylhydrazines A may be heated with imidate esters B with an appropriate base such as iPr₂NEt to effect a condensation to provide a 1,2,4-triazole product. Radical r^{b} may be one of the groups defined for R³, and compounds of Formula I are generated directly. Alternatively, r^{b} may be a halogen, in which case, intermediates C are obtained.

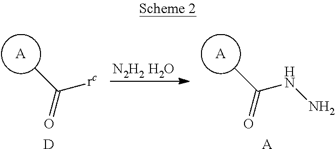

Acyl hydrazines A may be prepared from precursor D, which may be an ester (r^{c}=O-alkyl), activated chloride (r^{c}=Cl), or carboxylic acid (r^{c}=OH) as outlined in Scheme 2. When D is an ester, A may be prepared by heating D with excess hydrazine hydrate in an alcoholic solvent. When D is an acid chloride, A may be prepared by reacting with an excess of hydrazine hydrate and a base like iPr₂NEt, or A may be prepared by reacting by reacting with a protected hydrazine, such as Boc-hydrazine, followed by deprotection, such as by treatment with acid. When D is an carboxylic acid, D may be activated, for example with a carbodiimide such as 1-ethyl-3-(3-dimethylaminopropyl)carbodiimide or diisopropylcarbodiimide, and an activating group such as 4-dimethylaminopyridine, hydroxybenzotriazole, or pentafluorophenol. Treatment of the in-situ generated acyl-transfer reagent with hydrazine hydrate then provides A.

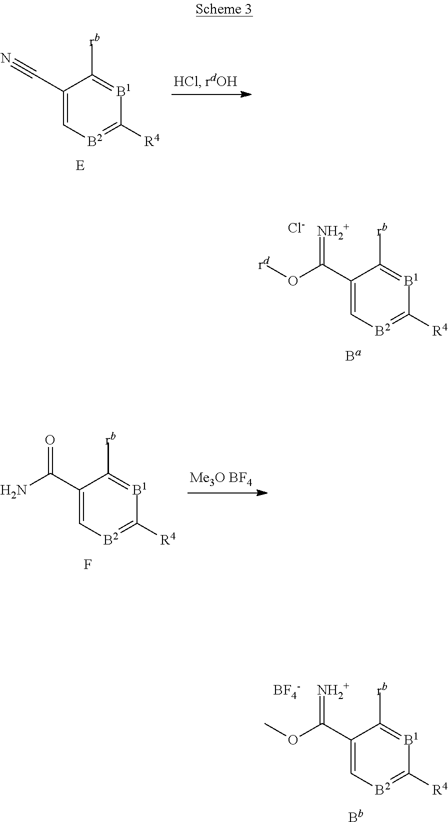

Imidate esters B may be prepared by the routes outlined in Scheme 3. Nitrile compounds E may be reacted with HCl and near stoichiometric amounts of alkyl alcohols r^{d}OH, wherein r^{d} is alkyl, to provide imidate esters Ba as hydrochloride salts. Amides F may be reacted with Meerwein's salt (Me₃O BF₄) to provide methyl imidates B^{b} as tetrafluoroborate salts.

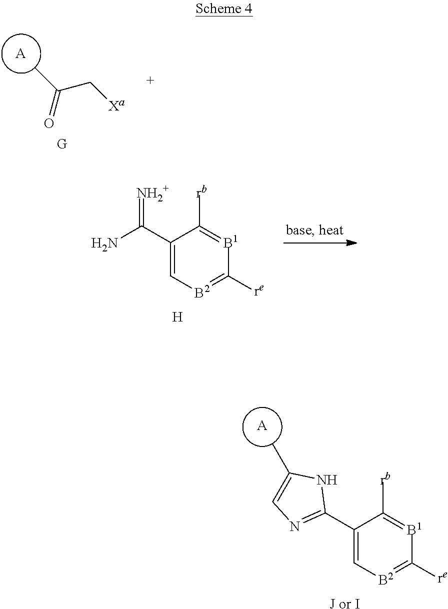

Scheme 4 outlines an exemplary route for the synthesis of compounds of Formula I when Y is “NH” and Z is “N.” 2-Haloketones G, wherein X^{a} is a halogen, may be heated with amidines B with an acid scavenger, such as iPr₂NEt, to provide an imidazole product. When r^{b} is one of the groups defined for R³ and r^{c} is one of the groups defined for R⁴, compounds of Formula I are generated directly. Alternatively, r^{b} may be a halogen and/or r^{e} may be a nitro group, in which case, intermediates J are obtained.

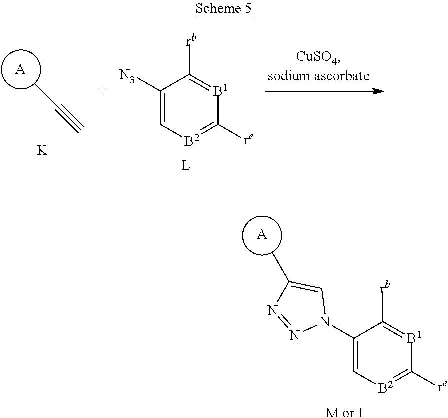

Scheme 5 outlines the synthesis of compounds of Formula I when Y is “CH” and X, Z, and W are “N.” Alkynes K and azides L may be reacted with a copper reagent, such as CuSO₄ and sodium ascorbate, to provide 1,2,3-triazoles M. When r^{b} is one of the groups defined for R³ and r^{c} is one of the groups defined for R⁴, compounds of Formula I are generated directly. Alternatively, r^{b} may be a halogen and/or r^{e} may be a nitro group, in which case, intermediates M are obtained.

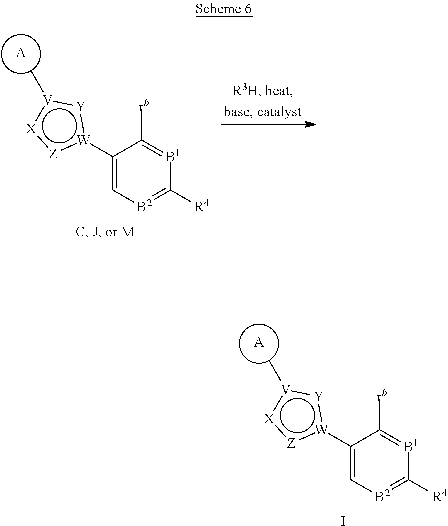

Schemes 6 and 7 describes derivatization of intermediates C, J, and M to provide compounds of Formula I. Scheme 6 illustrates derivatization of intermediates C, J, and M, when r^{b} is halogen; Scheme 7 illustrates derivatization of intermediates C, J, and M, when r^{e} is nitro. In Scheme 6, when r^{b} of intermediates C, J, or M is an F or C₁, the reaction of the intermediates with an amine (R³H) or amine hydrochloride (R³H HCl), in the presence of an appropriate base, such as iPr₂NEt, Et₃N, or K₂CO₃ provides compounds of Formula I via an S_{N}Ar reaction. When R^{b} is a bromine or iodine, compounds of Formula I may be prepared by cross-coupling by reacting C, J, or M and R³H in the presence of a base and suitable catalyst, typically derived from a palladium salt such as Pd(OAc)₂ or Pd(dba)₂ and a hindered phosphine ligand such at tri(tert-butyl)phosphine or 2,2′-bis-(diphenylphosphino)-1,1′-binaphthyl.

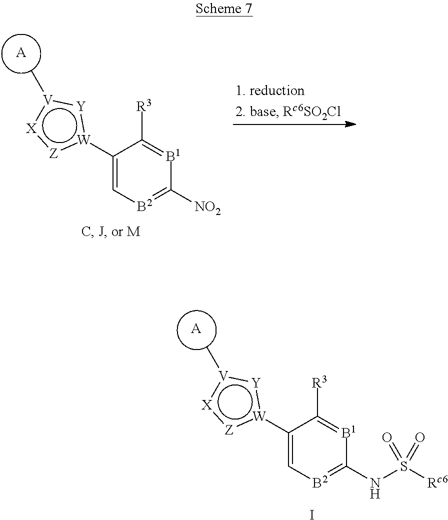

In Scheme 7, when r^{e} of intermediates C, J, or M is a nitro, the nitro group may be reduced to an aniline, for example with H₂ gas and catalyst like Pd/C, or with zinc and acetic acid. The aniline may be reacted with a sulfonyl chloride (R⁶SO₂Cl) and an acid scavenger such as iPr₂NEt to provide compounds of Formula I where R⁴ is —NHS(O)₂R^{c6}.

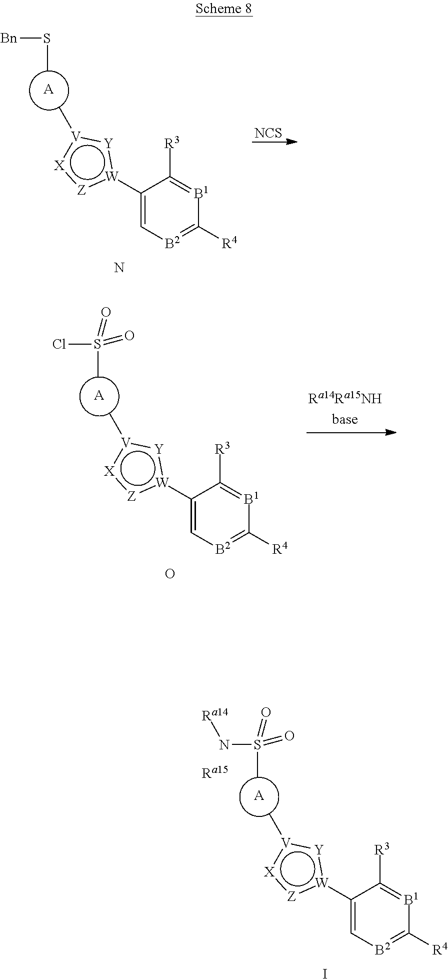

As shown in Scheme 8, the substituents on ring A may also carry precursors to substituents R¹. In which case, compounds of Formula I may be prepared by transformation of those precursors. For example, a thioether N may be converted to a sulfonyl chloride O by reaction with N-chlorosuccinimide (NCS), and O may be converted to a compound of Formula I having a sulfonamide R¹, by reaction with an amine and suitable base, such as iPr₂NEt.

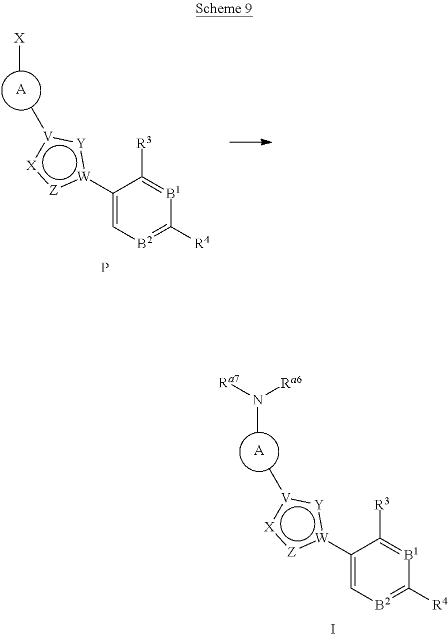

Another example of modification of substituents on ring A is described in Scheme 9. For compounds of structure P where X is an F or Cl, the reaction of the intermediates with an amine (R^{a7}R^{a6}NH), in the presence of an appropriate base, such as iPr₂NEt, Et₃N, or K₂CO₃ provides compounds of Formula I via an S_{N}Ar reaction. When X is a bromine or iodine, compounds of Formula I may be prepared by cross-coupling by reacting P and R^{a7}R^{a6}NH in the presence of a base and suitable catalyst, typically derived from a palladium salt such as Pd(OAc)₂ or Pd(dba)₂ and a hindered phosphine ligand such at tri(tert-butyl)phosphine or 2,2′-bis-(diphenylphosphino)-1,1′-binaphthyl.

### Enumerated Embodiments

The following enumerated embodiments are representative of some aspects of the invention.

A1. A compound of formula (I):

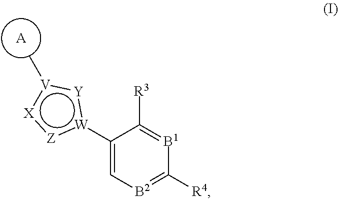

or a pharmaceutically acceptable salt thereof, wherein:
X and Z are independently O, N, or CH;
Y is NH or CH;
V and W are independently N or C;
wherein at least one of X and Z is N or Y is NH;
Ring A is

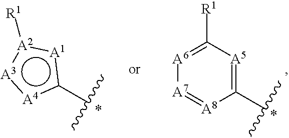

wherein
one or two of A¹, A³, and A⁴ are independently N, O, or S, and the remaining one or two of A¹, A³, and A⁴ are independently CH or CR²;
A² is N or C;
A⁵-A⁸ are independently CH, CR² or N, wherein at least two of A⁵, A⁶, A⁷, and A⁸ are CH or CR², and the remaining one or two of A⁵, A⁶, A⁷, and A⁸, if present, are N;
wherein “*” indicates the point of attachment to V;
B¹ and B² are each independently N or CH;
R¹ is C₁₋₆ alkyl, C₃₋₆cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆cycloalkyl of R¹ is optionally substituted with one or more substituents independently selected from the group consisting of halogen; and wherein the 3- to 10-membered heterocycloalkyl of R¹ is optionally substituted with one or more halogens;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl;
or R^{a14} and R^{a15} are taken together with the nitrogen to which they are attached to form a 3-to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein R^{b1} and R^{b2} are independently optionally substituted with C₁-C₃ alkyl or R^{1a1} and R^{1a2} are taken together with the nitrogen to which they are attached to form a 3-6 membered ring; or
R¹ and the R² of A⁴ are taken together with the carbon atoms to which they are attached to form a C₃-C₆ cycloalkyl or a 3- to 6-membered heterocycloalkyl;
R³ is piperidinyl, pyrrolidinyl, or azepinyl, wherein the piperidinyl, the pyrrolidinyl, or the azepinyl is optionally substituted with a C₃₋₁₀ cycloalkyl or 3- to 10-membered heterocycloalkyl,
wherein the C₃₋₁₀ cycloalkyl or 3- to 10-membered heterocycloalkyl forms a spirocyclic or fused bicyclic ring system with the piperidinyl, pyrrolidinyl, or the azepinyl, and wherein the piperidinyl, the pyrrolidinyl, the azepinyl or the spirocyclic or fused bicyclic ring system formed by the C₃₋₁₀ cycloalkyl or 3- to 10-membered heterocycloalkyl with piperidinyl, pyrrolidinyl, or the azepinyl is optionally substituted with one or more substituents independently selected from the group consisting of C₁-C₃ alkyl and C₁-C₃ haloalkyl;
R⁴ is hydrogen, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

A2. The compound of embodiment A1, or a pharmaceutically acceptable salt thereof, wherein X is N.

A3. The compound of embodiment A1 or A2, or a pharmaceutically acceptable salt thereof, wherein Z is N.

A4. The compound of any one of embodiments A1-A3, or a pharmaceutically acceptable salt thereof, wherein Y is NH.

A5. The compound of embodiment A1, or a pharmaceutically acceptable salt thereof, wherein the ring

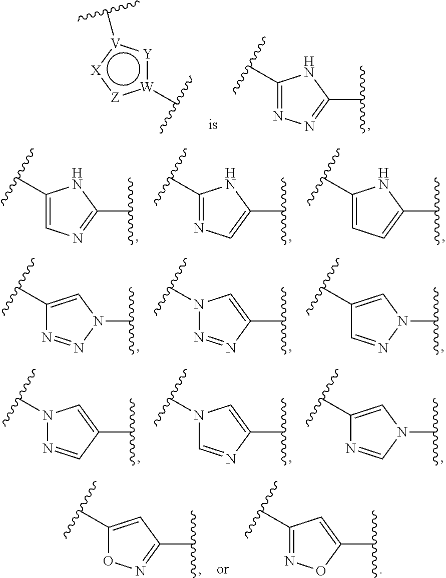

A6. The compound of embodiment A1, or a pharmaceutically acceptable salt thereof, wherein the ring

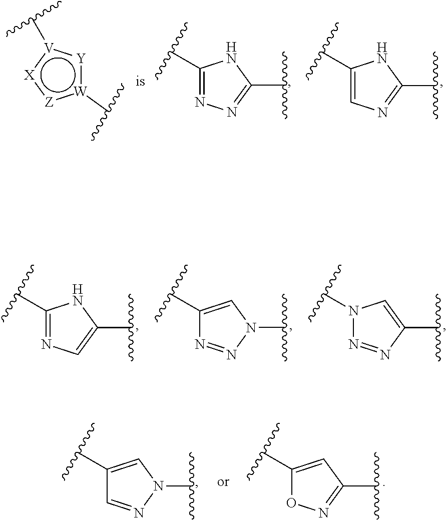

A7. The compound ofany one of embodiments A1-A6, or a pharmaceutically acceptable salt thereof, wherein Ring A is

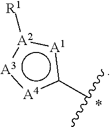

A8. The compound of any one of embodiments A1-A7, or a pharmaceutically acceptable salt thereof, wherein Ring A is

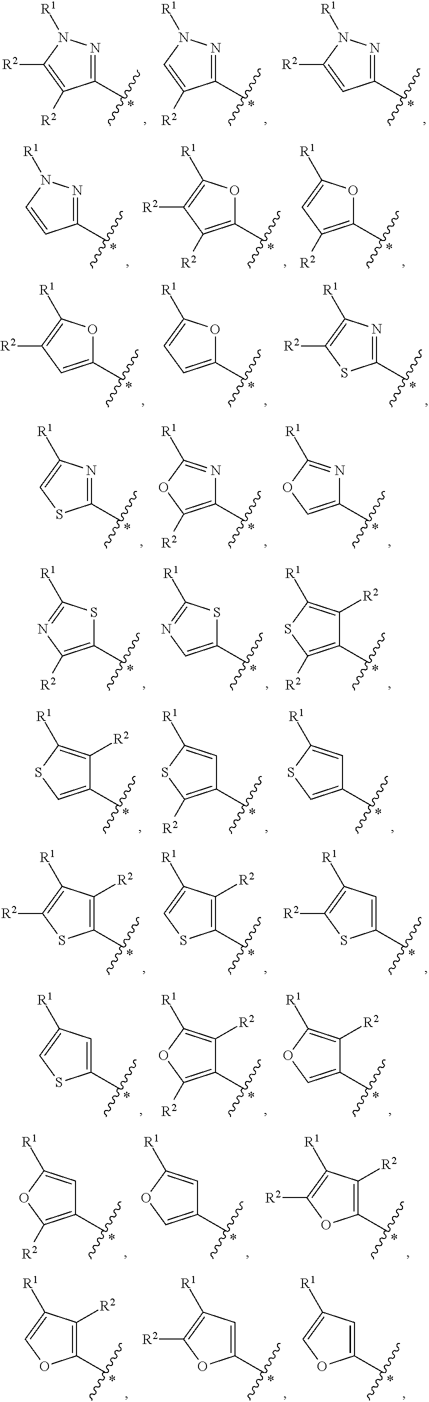

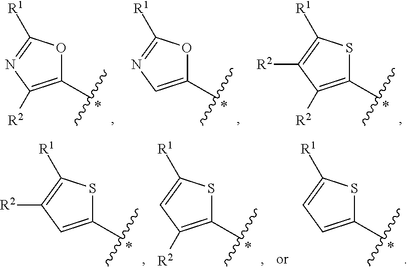

A9. The compound of an one of embodiments A1-A8, or a pharmaceutically acceptable salt thereof, wherein Ring A is

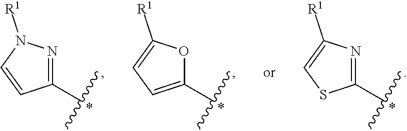

A10. The compound of any one of embodiments A1-A6, or a pharmaceutically acceptable salt thereof, wherein Ring A is

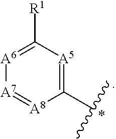

A11. The compound of any one of embodiments A1-A6 and A10, or a pharmaceutically acceptable salt thereof, wherein Ring A is

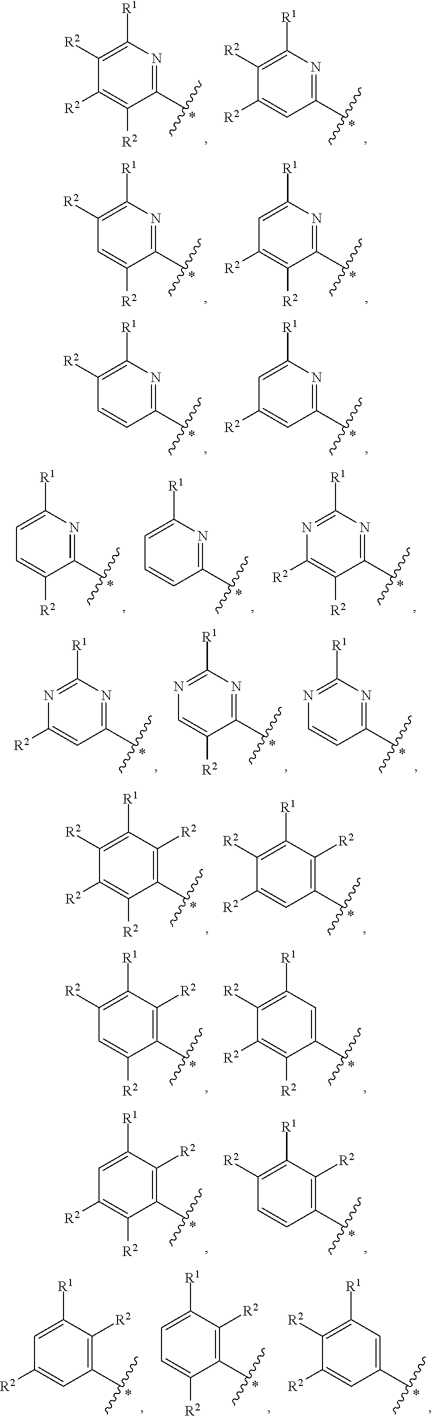

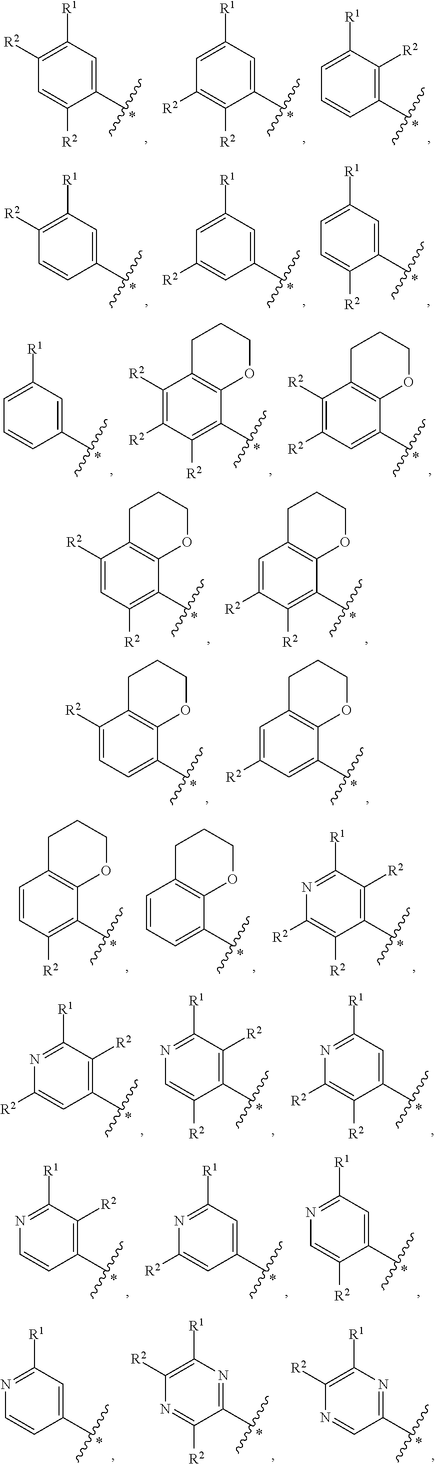

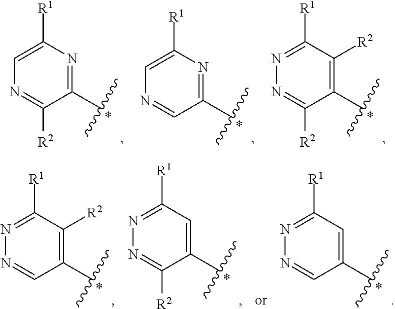

A12. The compound of any one of embodiments A1-A6 and A10-A11, or a pharmaceutically acceptable salt thereof, wherein Ring A is

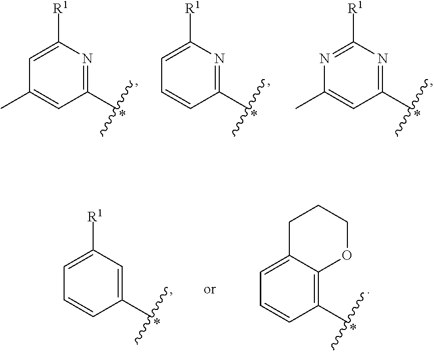

A13. The compound of any one of embodiments A1-A12, or a pharmaceutically acceptable salt thereof, wherein R¹ is C₁₋₆ alkyl, C₃₋₆cycloalkyl, 3- to 10-membered heterocycloalkyl, —NR^{a6}R^{a7}, —S(O)₂NR^{a14}R^{a15}, or —S(O)₂R^{a16}, wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of Rr is optionally substituted with one or more substituents independently selected from the group consisting of halogen; and wherein the 3- to 10-membered heterocycloalkyl of R is optionally substituted with one or more halogens.

A14. The compound of any one of embodiments A1-A13, or a pharmaceutically acceptable salt thereof, wherein R¹ is

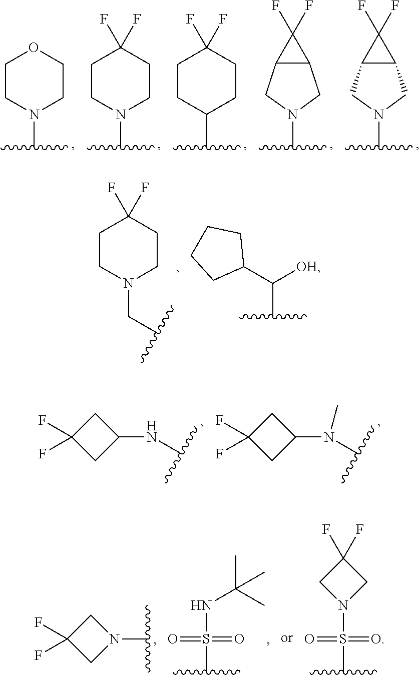

A15. The compound of any one of embodiments A1-A14 or a pharmaceutically acceptable salt thereof, wherein the ring is

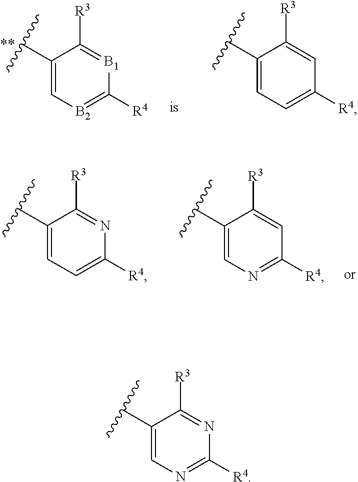

A16. The compound of any one of embodiments A1-A15, or a pharmaceutically acceptable salt thereof, wherein the ring

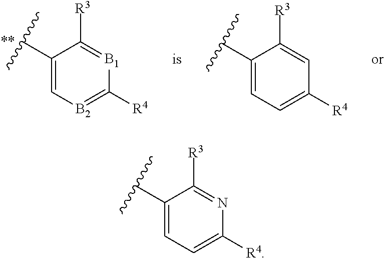

A17. The compound of any one of embodiments A1-A15, or a pharmaceutically acceptable salt thereof, wherein R³ is piperidinyl, wherein the piperidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, and wherein the piperidinyl or the spirocyclic or fused bicyclic ring system formed by the C₃₋₁₀ cycloalkyl or 3- to 10-membered heterocycloalkyl with piperidinyl is optionally substituted with one or more substituents independently selected from the group consisting of C₁-C₃ alkyl and C₁-C₃ haloalkyl.

A18. The compound of any one of embodiments A1-A16, or a pharmaceutically acceptable salt thereof, wherein R³ is

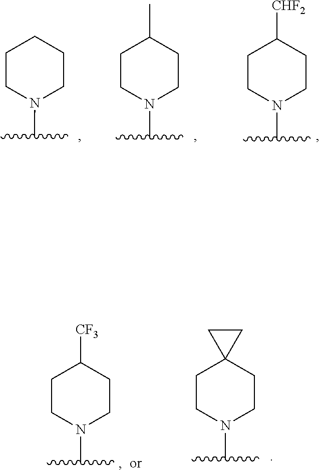

A19. The compound of any one of embodiments A1-A15, or a pharmaceutically acceptable salt thereof, wherein R³ is pyrrolidinyl, wherein the pyrrolidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the pyrrolidinyl, and
wherein the pyrrolidinyl or the spirocyclic or fused bicyclic ring system formed by the C₃₋₁₀ cycloalkyl or 3- to 10-membered heterocycloalkyl with pyrrolidinyl is optionally substituted with one or more substituents independently selected from the group consisting of C₁-C₃ alkyl and C₁-C₃ haloalkyl.

A20. The compound of any one of embodiments A1-A15 and A19, or a pharmaceutically acceptable salt thereof, wherein R³ is

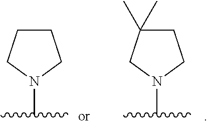

A21. The compound of any one of embodiments A1-A15, or a pharmaceutically acceptable salt thereof, wherein R³ is azepinyl, wherein the azepinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the azepinyl, and
wherein the azepinyl or the spirocyclic or fused bicyclic ring system formed by the C₃₋₁₀ cycloalkyl or 3- to 10-membered heterocycloalkyl with azepinyl is optionally substituted with one or more substituents independently selected from the group consisting of C₁-C₃ alkyl and C₁-C₃ haloalkyl.

A22. The compound of any one of embodiments A1-A15 and A21, or a pharmaceutically acceptable salt thereof, wherein R³ is

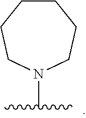 [SMILES: *N1CCCCCC1]

A23. The compound of any one of embodiments A1-A22, or a pharmaceutically acceptable salt thereof, wherein R⁴ is hydrogen, halo, or —NR^{c5}S(O)₂R^{c6}.

A24. The compound of any one of embodiments A1-A23 or a pharmaceutical acceptable salt thereof, wherein R⁴ is

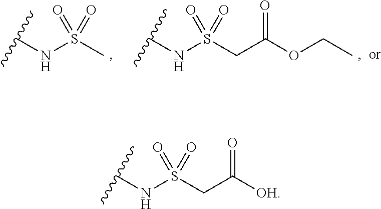

A25. The compound of any one of embodiments A1-A24, or a pharmaceutically acceptable salt thereof, wherein R⁴ is

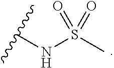 [SMILES: *NS(C)(=O)=O]

A26. The compound of embodiment A1, or a pharmaceutically acceptable salt thereof, wherein the compound is selected from the group consisting of compounds 1 to 48 of Table 1.

A27. A pharmaceutical composition comprising a compound of any one of embodiments A1-A26, or a pharmaceutically acceptable salt thereof, and a pharmaceutically acceptable carrier or excipient.

A28. A method of inhibiting KIF18A comprising contacting a cell with an effective amount of a compound of any one of embodiments A1-A26, or a pharmaceutically acceptable salt thereof, or a pharmaceutical composition of embodiment A27.

A29. A method of treating a disease or condition mediated by KIF18A in a subject in need thereof, comprising administering to the subject a therapeutically effective amount of a compound of any one of embodiments A1-A26, or a pharmaceutically acceptable salt thereof, or a pharmaceutical composition of embodiment A27.

A30. A method of treating cancer in a subject in need thereof, comprising administering to the subject a therapeutically effective amount of a compound of any one of embodiments A1-A26, or a pharmaceutically acceptable salt thereof, or a pharmaceutical composition of embodiment A27.

A31. The method of embodiment A30, wherein the cancer is selected from the group consisting of carcinomas, cancer of the anus, bladder, breast, colon, small intestine, appendix, kidney, renal pelvis, ureter, urothelium, liver, lung, pleura, esophagus, head and neck, nasopharynx, oropharynx, hypopharynx, oral cavity, larynx, biliary tract, gall-bladder, ovary, testicle, germ cell, uterus, pancreas, stomach, cervix, thyroid, prostate, salivary gland, or skin, hematopoietic tumors of lymphoid lineage, hematopoietic tumors of myeloid lineage, hematopoietic tumors of any lineage, myeloma, tumors of mesenchymal origin including sarcomas, tumors of the central and peripheral nervous system, tumor of neuroendocrine origin, tumor of endocrine origin, small cell tumors, tumors of unknown primary, other tumors comprising retinoblastoma, melanoma, seminoma, teratocarcinoma, osteosarcoma, and other cancer-related disorders that are a consequence of cancer presence or progression.

B1. A compound of formula (I):

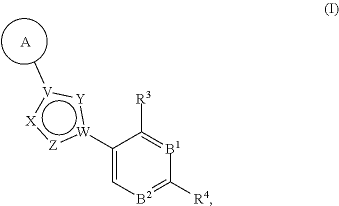

or a pharmaceutically acceptable salt thereof, wherein:
X and Z are independently O, N, or CH;
Y is NH or CH;
V and W are independently N or C;
   wherein at least one of X and Z is N or Y is NH;
Ring A is

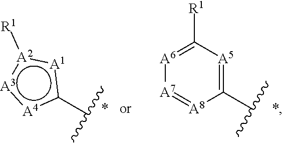

wherein
one, two, or three of A¹, A³, and A⁴ are independently N, NR^{a}, O, or S, and the remaining one or two of A¹, A³, and A⁴, if present, are independently CH or CR², wherein RAI is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A1} is =0;
wherein “*” indicates the point of attachment to V;
B¹ and B² are each independently N, CH or CR^{B}, wherein R^{B} is halogen;
R¹ is C₁₋₆ alkyl, C₃₋₆cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3-6 membered ring; or
R¹ and the R² of A⁵ are taken together with the carbon atoms to which they are attached to form a C₃-C₆ cycloalkyl or a 3- to 10-membered heterocycloalkyl;
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or azepanyl, or
   wherein the piperidinyl, pyrrolidinyl, or azepanyl are optionally substituted with a C₁₋₂ alkylene to form a bridged piperidinyl, pyrrolidinyl, or azepanyl ring system,
   wherein the piperidinyl, the pyrrolidinyl, the azepanyl, or the spirocyclic, fused, or bridged bicyclic ring system formed by the C₃₋₁₀ cycloalkyl or 3- to 10-membered heterocycloalkyl with piperidinyl, pyrrolidinyl, or azepanyl is optionally substituted with one or more substituents independently selected from the group consisting of C₁-C₃ alkyl and C₁-C₃ haloalkyl;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH.

B2. The compound of embodiment B1, or a pharmaceutically acceptable salt thereof, wherein X is N.

B3. The compound of embodiment B1 or B2, or a pharmaceutically acceptable salt thereof, wherein Z is N.

B4. The compound of any one of embodiments B1-B3, or a pharmaceutically acceptable salt thereof, wherein Y is NH.

B5. The compound of embodiment B1, or a pharmaceutically acceptable salt thereof, wherein the ring

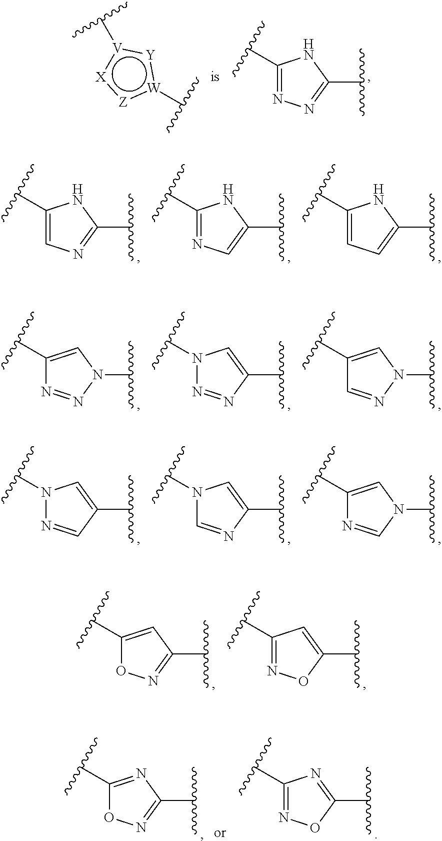

B6. The compound of embodiment B1, or a pharmaceutically acceptable salt thereof, wherein the ring

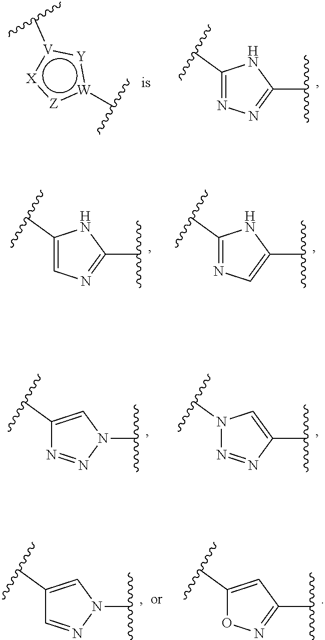

B7. The compound of embodiment B1, or a pharmaceutically acceptable salt thereof, wherein the compound of Formula (I) is a compound of Formula (I-a):

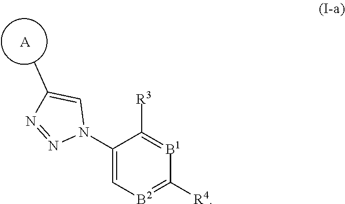

B8. The compound of embodiment B1, or a pharmaceutically acceptable salt thereof, wherein the compound of Formula (I) is a compound of Formula (I-b):

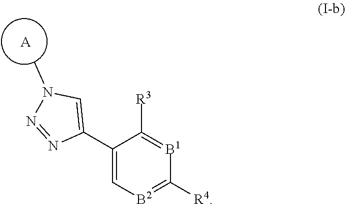

B9. The compound of embodiment B1, or a pharmaceutically acceptable salt thereof, wherein the compound of Formula (I) is a compound of Formula (I-c):

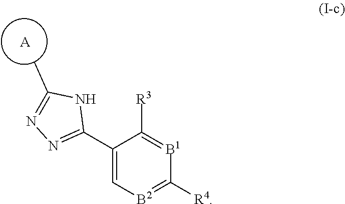

B10. The compound of any one of embodiments B1-B9, or a pharmaceutically acceptable salt thereof, wherein Ring A is

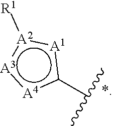

B11. The compound of any one of embodiments B1-B10, or a pharmaceutically acceptable salt thereof, wherein Ring A is

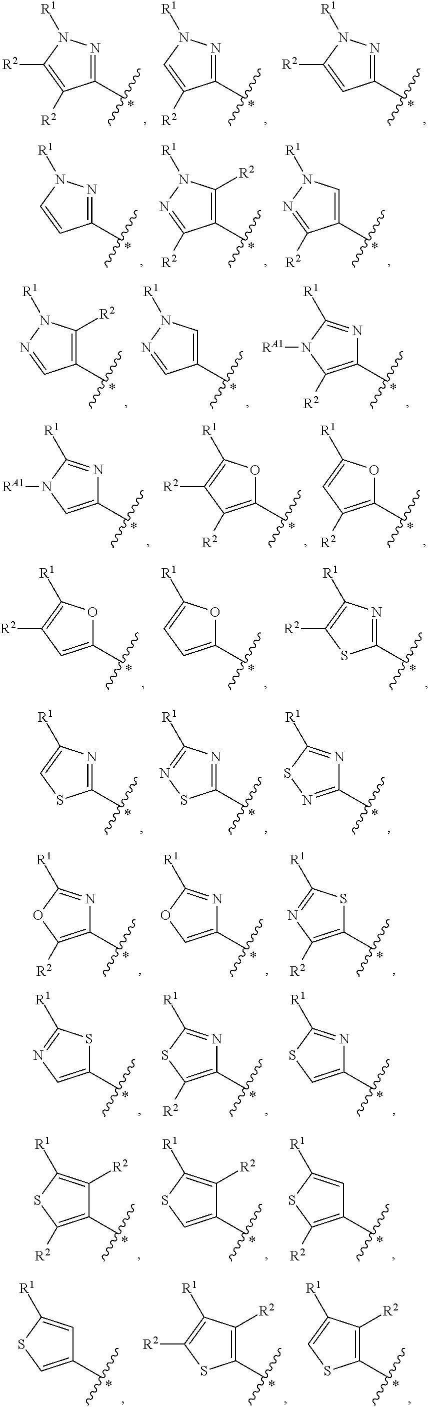

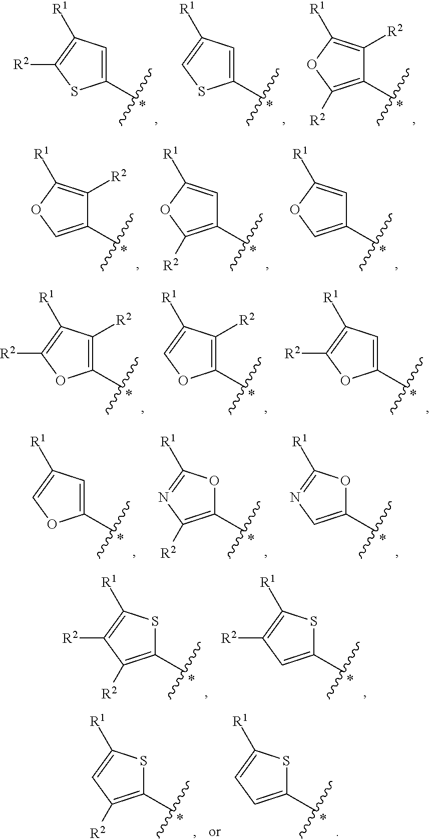

B12. The compound of any one of embodiments B1-B11, or a pharmaceutically acceptable salt thereof, wherein Ring A is

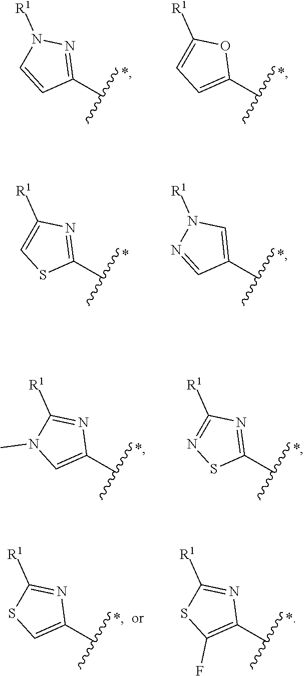

B13. The compound of any one of embodiments B1-B9, or a pharmaceutically acceptable salt thereof, wherein Ring A is

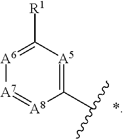

B14. The compound of any one of embodiments B1-B9 and B13, or a pharmaceutically acceptable salt thereof, wherein Ring A is

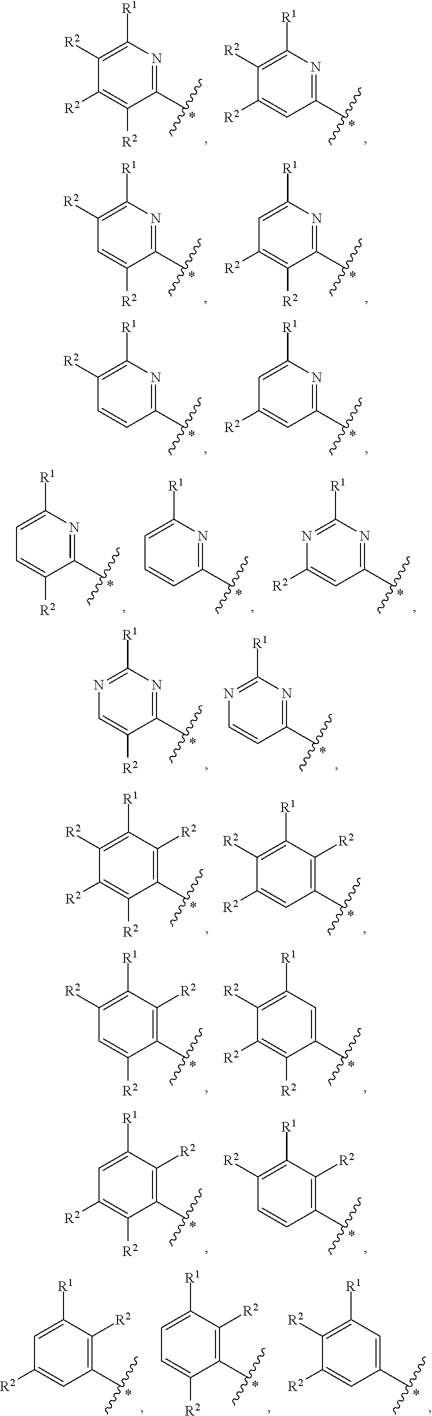

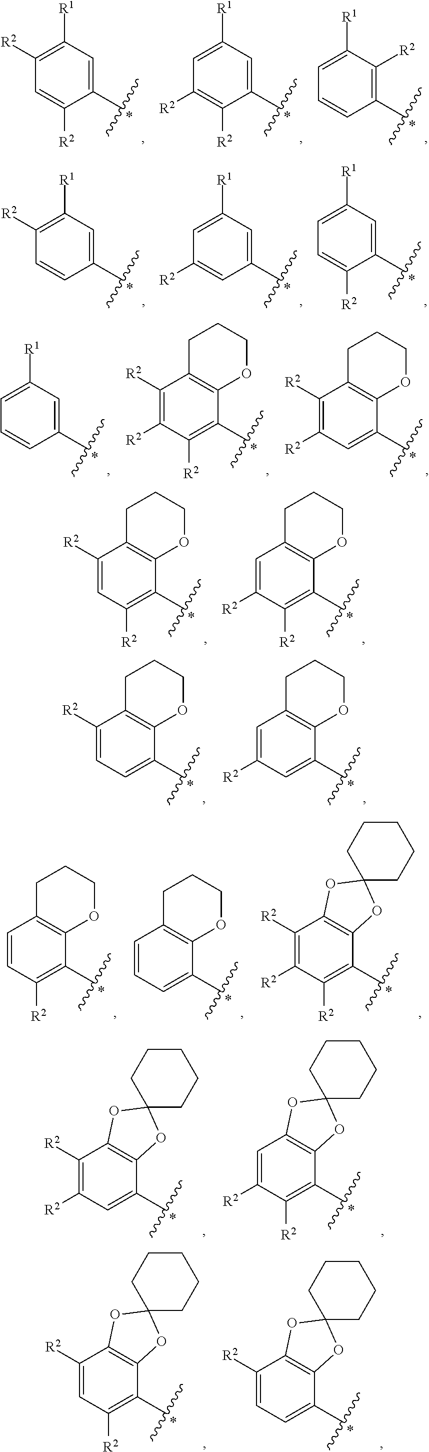

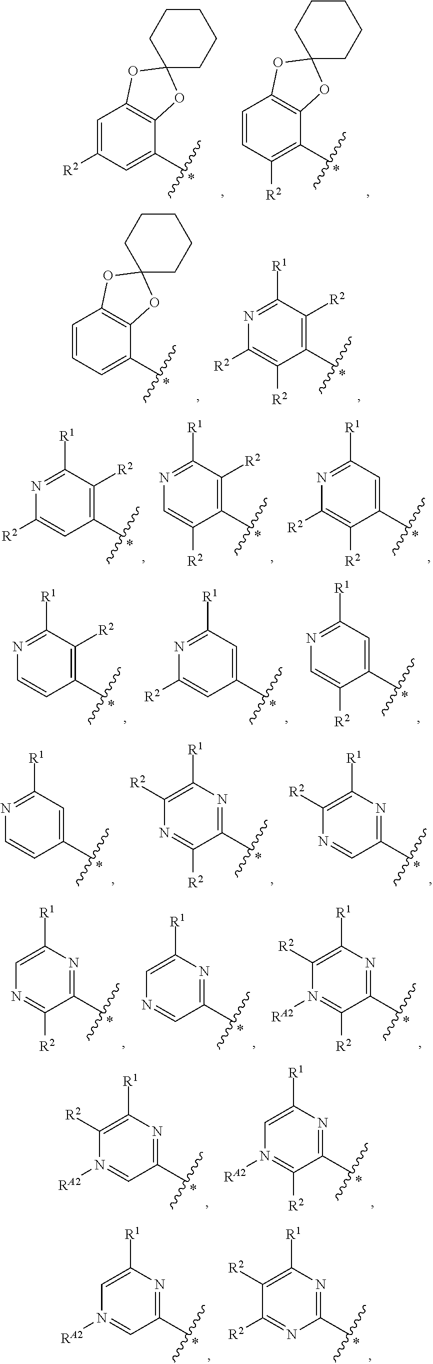

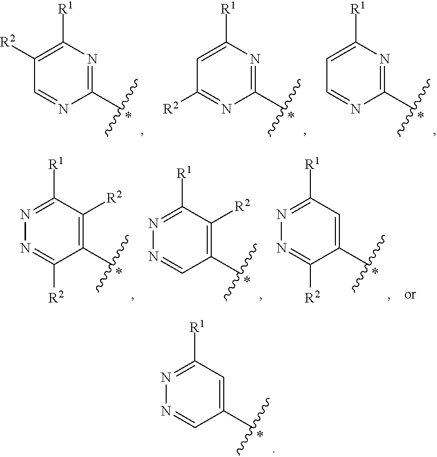

B15. The compound of any one of embodiments B1-9 and B13-B14, or a pharmaceutically acceptable salt thereof, wherein Ring A is

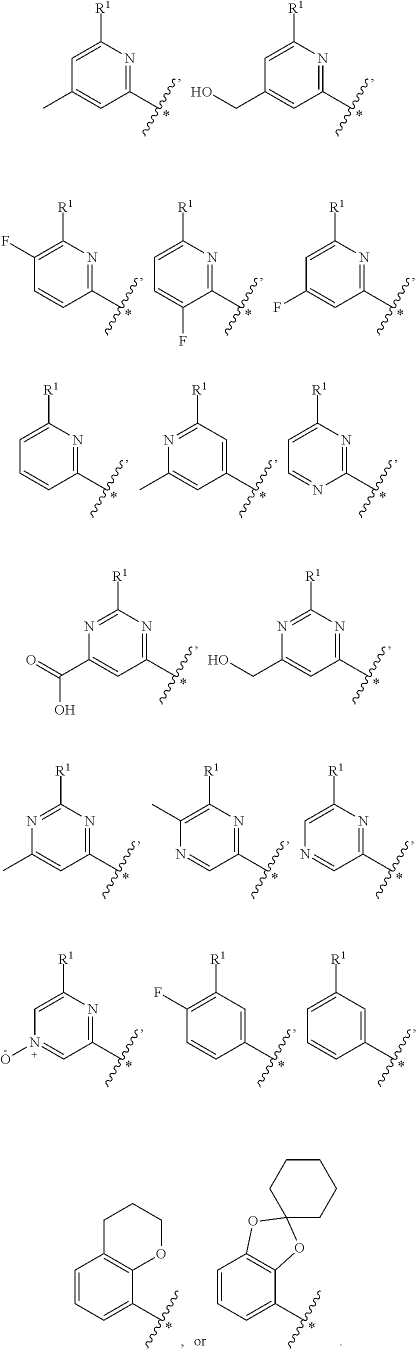

B16. The compound of any one of embodiments B1-B15, or a pharmaceutically acceptable salt thereof, wherein R¹ is C₁₋₆ alkyl, C₃₋₆cycloalkyl, C₃₋₁₀ cycloalkenyl, 3- to 10-membered heterocycloalkyl, —NR^{a6}R^{a7}, —OR^{a10}, —S(O)₂NR^{a14}R^{a15}, or —S(O)₂R^{a16}, wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl.

B17. The compound of any one of embodiments B1-B16, or a pharmaceutically acceptable salt thereof, wherein R¹ is

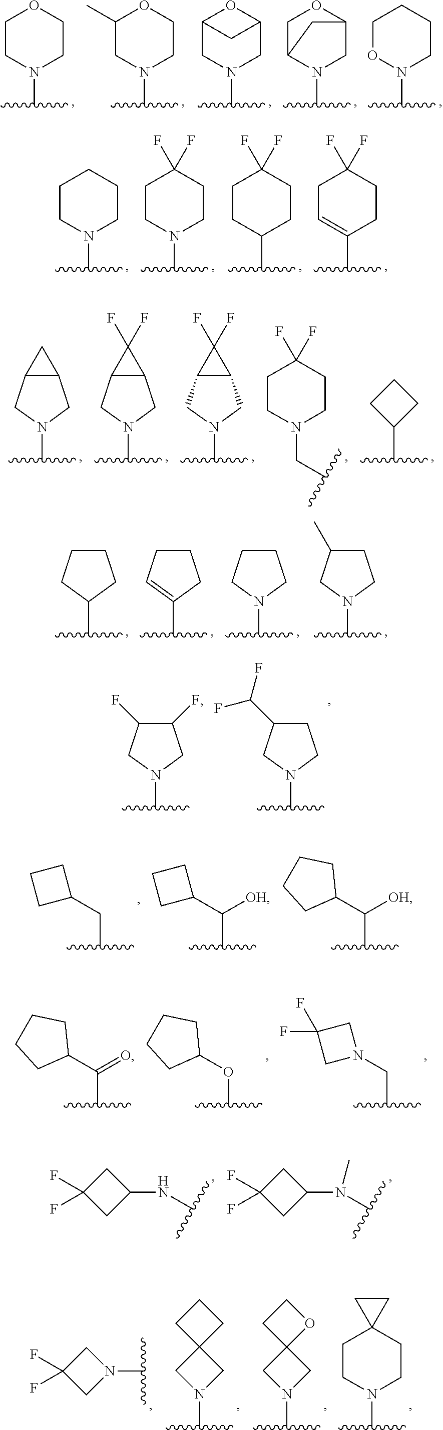

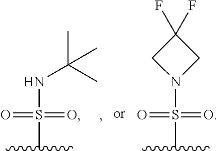

B18. The compound of any one of embodiments B1-B17 or a pharmaceutically acceptable salt thereof, wherein the ring

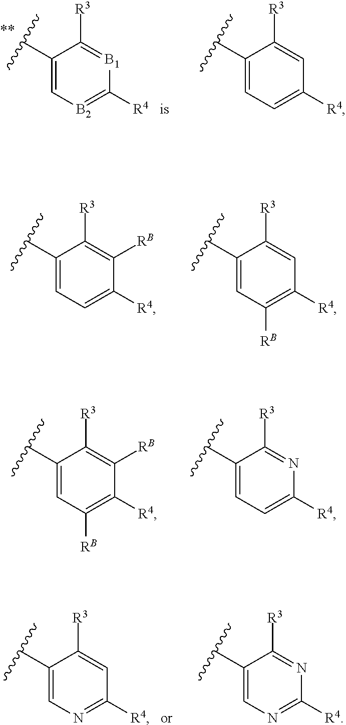

B19. The compound of any one of embodiments B1-B18, or a pharmaceutically acceptable salt thereof, wherein the ring

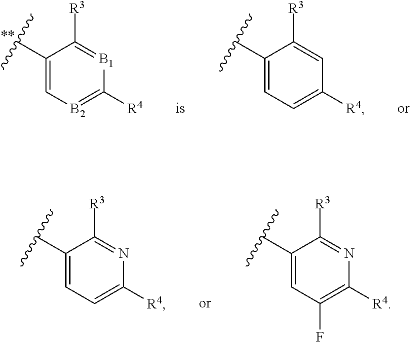

B20. The compound of any one of embodiments B1-B19, or a pharmaceutically acceptable salt thereof, wherein R3 is piperidinyl, wherein the piperidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, or wherein the piperidinyl is optionally substituted with a C₁₋₂ alkylene to form a bridged piperidinyl ring system, wherein the piperidinyl, or the spirocyclic, fused, or bridged bicyclic ring system formed by the C₃₋₁₀ cycloalkyl, 3- to 10-membered heterocycloalkyl, or C₁₋₂ alkylene with piperidinyl is optionally substituted with one or more substituents independently selected from the group consisting of C₁-C₃ alkyl and C₁-C₃ haloalkyl.

B21. The compound of any one of embodiments B1-B20, or a pharmaceutically acceptable salt thereof, wherein R³ is

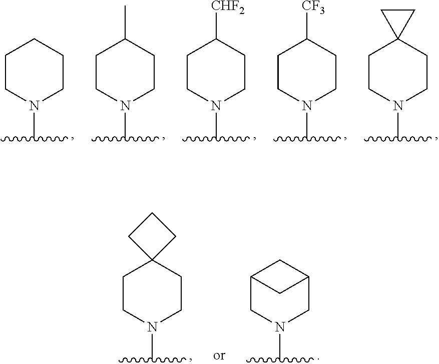

B22. The compound of any one of embodiments B1-B19, or a pharmaceutically acceptable salt thereof, wherein R³ is pyrrolidinyl, wherein the pyrrolidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the pyrrolidinyl, or wherein the pyrrolidinyl is optionally substituted with a C₁₋₂ alkylene to form a bridged pyrrolidinyl ring system, wherein the pyrrolidinyl, or the spirocyclic, fused, or bridged bicyclic ring system formed by the C₃₋₁₀ cycloalkyl, 3- to 10-membered heterocycloalkyl, or C₁₋₂ alkylene with the pyrrolidinyl is optionally substituted with one or more substituents independently selected from the group consisting of C₁-C₃ alkyl and C₁-C₃ haloalkyl.

B23. The compound of any one of embodiments B1-B19 and B22, or a pharmaceutically acceptable salt thereof, wherein R³ is

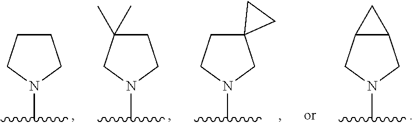

B24. The compound of any one of embodiments B1-B19, or a pharmaceutically acceptable salt thereof, wherein R3 is azepanyl, the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the azepanyl or wherein the azepanyl is optionally substituted with a C₁₋₂ alkylene to form a bridged azepanyl ring system, wherein the azepanyl or the spirocyclic, fused, or bridged bicyclic ring system formed by the C₃₋₁₀ cycloalkyl, 3- to 10-membered heterocycloalkyl, or C₁₋₂ alkylene with the azepanyl is optionally substituted with one or more substituents independently selected from the group consisting of C₁-C₃ alkyl and C₁-C₃ haloalkyl.

B25. The compound of any one of embodiments B1-B19 and B24, or a pharmaceutically acceptable salt thereof, wherein R³ is

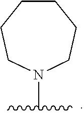 [SMILES: *N1CCCCCC1]

B26. The compound of any one of embodiments B1-B25 or a pharmaceutically acceptable salt thereof, wherein R⁴ is hydrogen, halo, or —NR^{c5}S(O)₂R^{c6}.

B27. The compound of any one of embodiments B1-B26, or a pharmaceutically acceptable salt thereof, wherein H, Br,

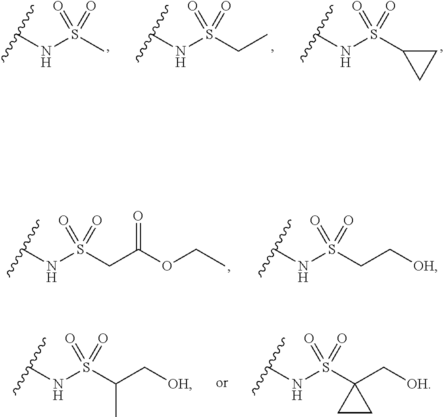

B28. The compound of any one of embodiments B1-B27, or a pharmaceutically acceptable salt thereof, wherein R⁴ is

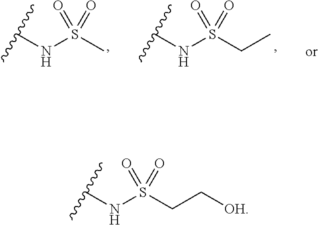

B29. A compound of formula (III)

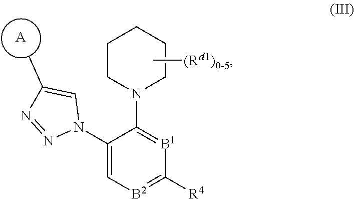

or a pharmaceutically acceptable salt thereof, wherein:
Ring A is

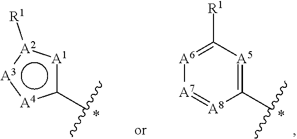

wherein
one, two, or three of A¹, A³, and A⁴ are independently N, NR^{a}, O, or S, and the remaining one or two of A¹, A³, and A⁴, if present, are independently CH or CR², wherein RAI is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2} wherein R^{A2} is ═O;
wherein “*” indicates the point of attachment to V;
B¹ and B² are each independently N, CH or CR^{B}, wherein Ra is halogen;
R¹ is C₁₋₆ alkyl, C₃₋₆cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
   wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3-6 membered ring; or
R¹ and the R² of A⁵ are taken together with the carbon atoms to which they are attached to form a C₃-C₆ cycloalkyl or a 3- to 10-membered heterocycloalkyl;
wherein each R^{d1} is independently selected from the group consisting of C₁-C₃ alkyl and C₁-C₃ haloalkyl; or wherein two R^{d1} are taken together to form a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl; or wherein two R^{d1} are taken together to form a C₁₋₂ alkylene, wherein the C₁₋₂ alkylene forms a bridged piperidinyl ring system,
   wherein the spirocyclic, fused, or bridged bicyclic ring system formed by the C₃₋₁₀ cycloalkyl, 3- to 10-membered heterocycloalkyl, or C₁₋₂ alkylene with the piperidinyl is optionally substituted with one or more substituents independently selected from the group consisting of C₁-C₃ alkyl and C₁-C₃ haloalkyl;
R⁴ is H, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, an d-C(O)—O—C₁-C₃ alkyl, and wherein each C₃₋₁₀ cycloalkyl is optionally substituted with one or more substituents independently selected from the group consisting of C₁-C₆ alkylene-OH.

B30. A compound of formula (II):

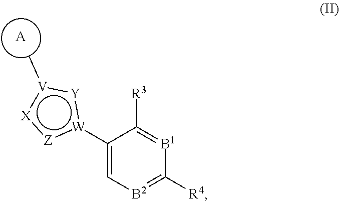

or a pharmaceutically acceptable salt thereof, wherein:
X and Z are independently O, N, or CH;
Y is NH or CH;
V and W are independently N or C;
wherein at least one of X and Z is N or Y is NH;
Ring A is

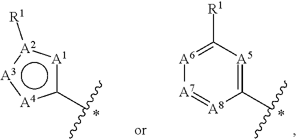

wherein
one or two of A¹, A³, and A⁴ are independently N, O, or S, and the remaining one or two of A¹, A³, and A⁴ are independently CH or CR²;
A² is N or C;
A⁵-A⁸ are independently CH, CR² or N, wherein at least two of A⁵, A⁶, A⁷, and A⁸ are CH or CR², and the remaining one or two of A⁵, A⁶, A⁷, and A⁸, if present, are N;
wherein “*” indicates the point of attachment to V;
B¹ and B² are each independently N or CH;
R¹ is C₁₋₆ alkyl, C₃₋₆cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆cycloalkyl of R¹ is optionally substituted with one or more substituents independently selected from the group consisting of halogen; and wherein the 3- to 10-membered heterocycloalkyl of R¹ is optionally substituted with one or more halogens;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl;
or R^{a14} and R^{a15} are taken together with the nitrogen to which they are attached to form a 3-to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein R^{b1} and R^{b2} are independently optionally substituted with C₁-C₃ alkyl or R^{b1} and R^{b2} are taken together with the nitrogen to which they are attached to form a 3-6 membered ring; or
R¹ and the R² of A⁵ are taken together with the carbon atoms to which they are attached to form a C₃-C₆ cycloalkyl or a 3- to 6-membered heterocycloalkyl;
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or the azepanyl, and
wherein the piperidinyl, the pyrrolidinyl, the azepanyl or the spirocyclic or fused bicyclic ring system formed by the C₃₋₁₀ cycloalkyl or 3- to 10-membered heterocycloalkyl with piperidinyl, pyrrolidinyl, or the azepanyl is optionally substituted with one or more substituents independently selected from the group consisting of C₁-C₃ alkyl and C₁-C₃ haloalkyl;
R⁴ is hydrogen, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

B31. The compound of embodiment B30, or a pharmaceutically acceptable salt thereof, wherein X is N.

B32. The compound of embodiment B30 or B31, or a pharmaceutically acceptable salt thereof, wherein Z is N.

B33. The compound of any one of embodiments B30-B32, or a pharmaceutically acceptable salt thereof, wherein Y is NH.

B34. The compound of embodiment B30, or a pharmaceutically acceptable salt thereof, wherein the ring

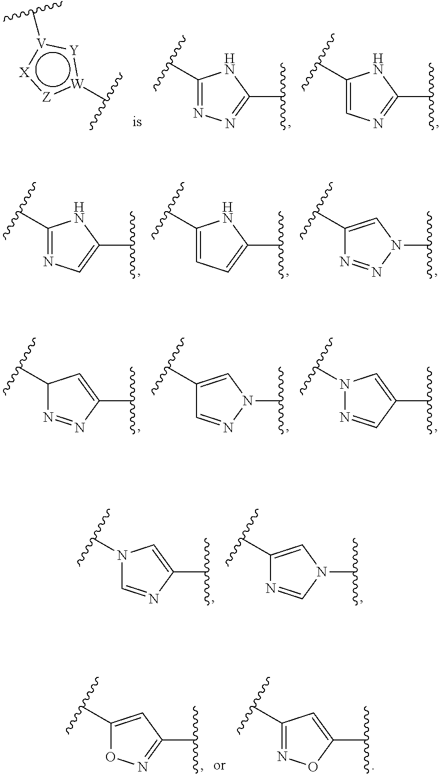

B35. The compound of embodiment B30, or a pharmaceutically acceptable salt thereof, wherein the ring

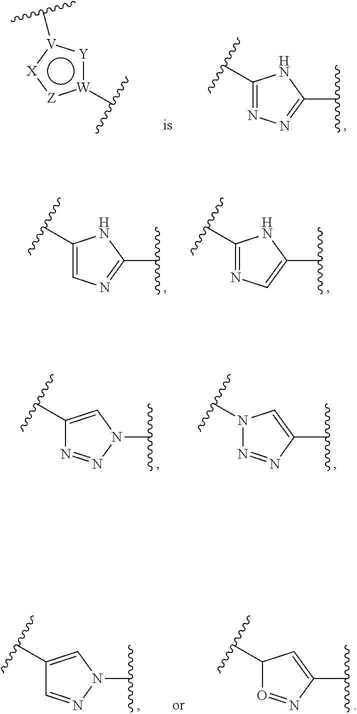

B36. The compound of embodiment B30, or a pharmaceutically acceptable salt thereof, wherein the compound of Formula (II) is a compound of Formula (II-a):

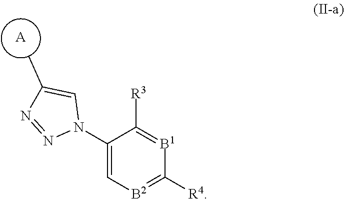

B37. The compound of embodiment B30, or a pharmaceutically acceptable salt thereof, wherein the compound of Formula (II) is a compound of Formula (II-b):

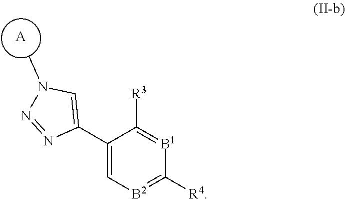

B38. The compound of embodiment B30, or a pharmaceutically acceptable salt thereof, wherein the compound of Formula (II) is a compound of Formula (II-c):

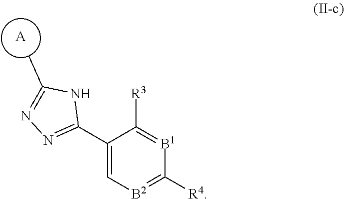

B39. The compound of any one of embodiments B30-B38, or a pharmaceutically acceptable salt thereof, wherein Ring A is

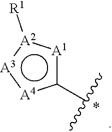

B40. The compound of any one of embodiments B30-B39, or a pharmaceutically acceptable salt thereof, wherein Ring A is

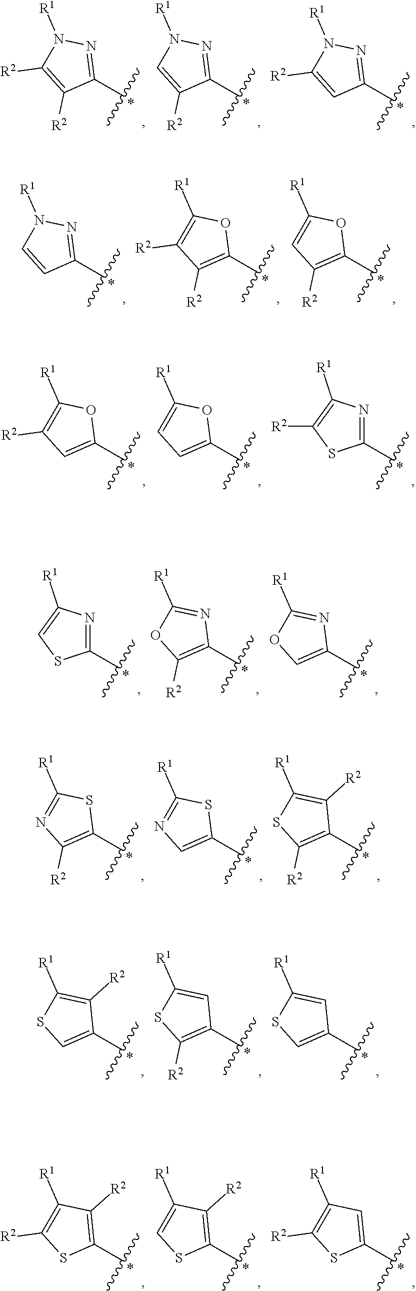

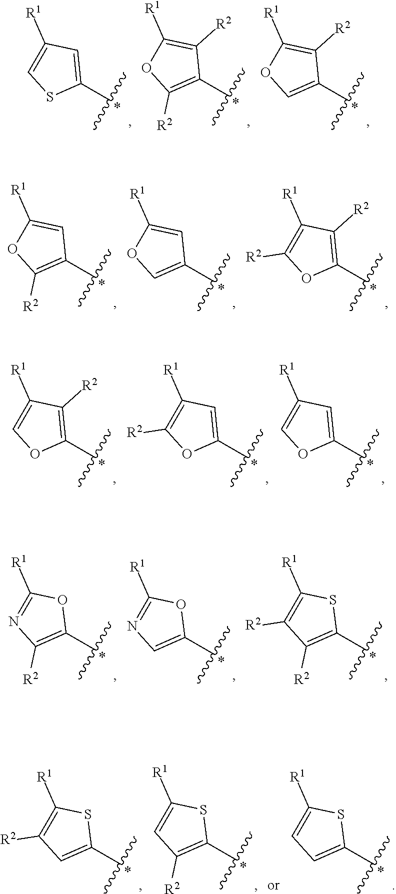

B41. The compound of any one of embodiments B30-B40, or a pharmaceutically acceptable salt thereof, wherein Ring A is

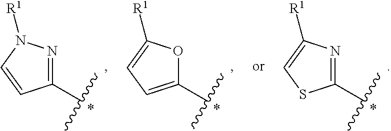

B42. The compound of any one of embodiments B30-B38, or a pharmaceutically acceptable salt thereof, wherein Ring A is

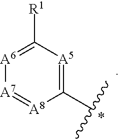

B43. The compound of any one of embodiments B30-B38 and B42, or a pharmaceutically acceptable salt thereof, wherein Ring A is

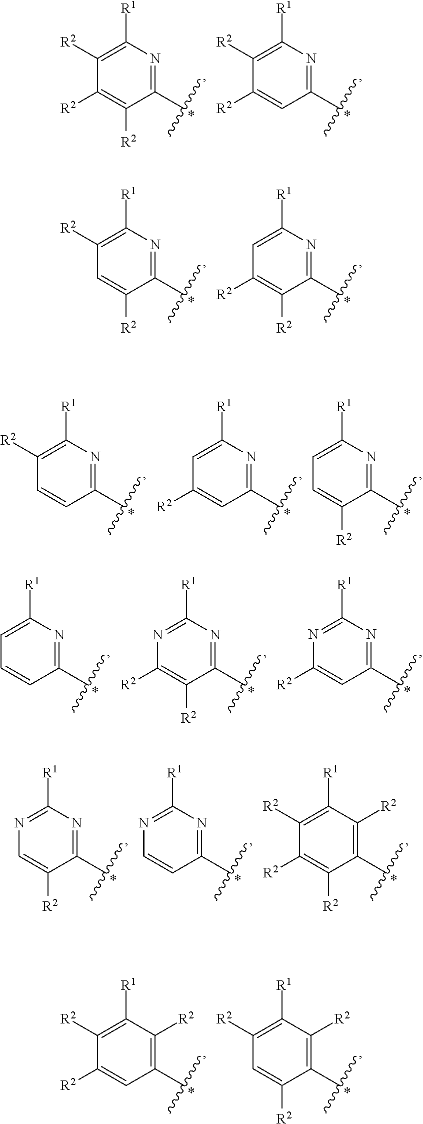

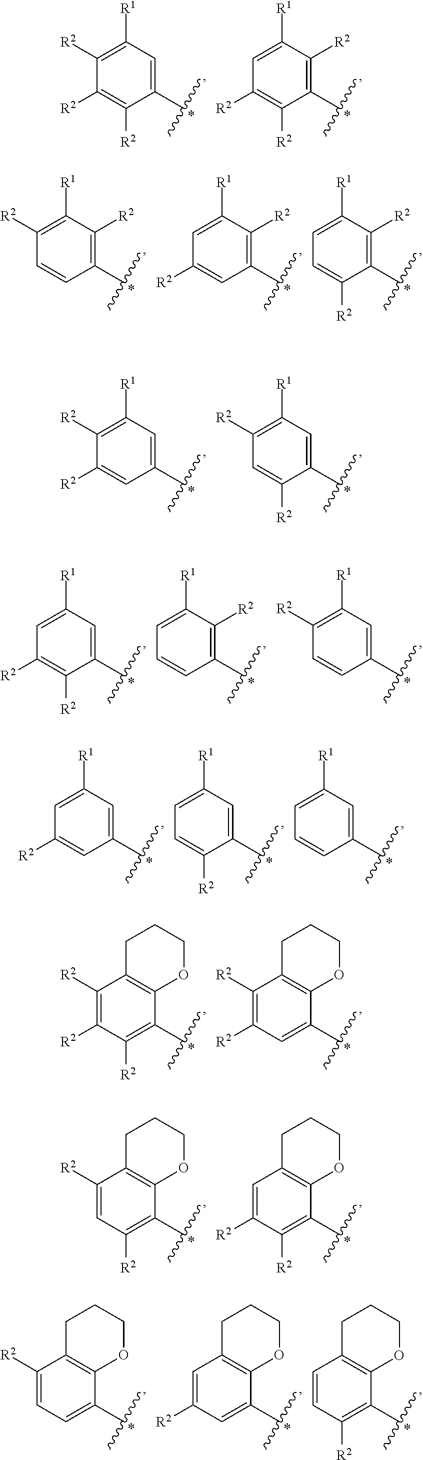

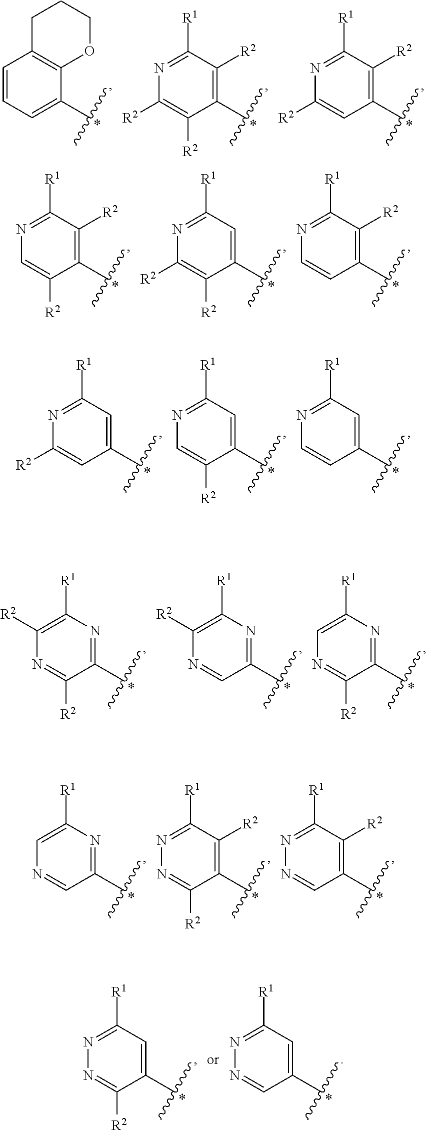

B44. The compound of any one of embodiments B30-B38 and B42-B43, or a pharmaceutically acceptable salt thereof, wherein Ring A is

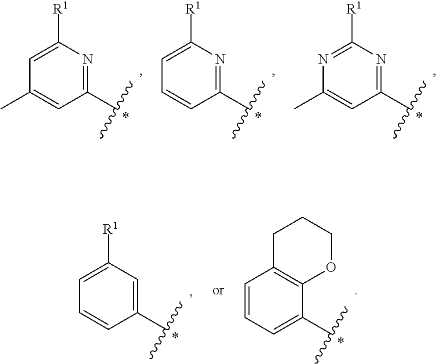

B45. The compound of any one of embodiments B30-B44, or a pharmaceutically acceptable salt thereof, wherein R¹ is C₁₋₆ alkyl, C₃₋₆ cycloalkyl, 3- to 10-membered heterocycloalkyl, —NR^{a6}R^{a7}, —S(O)₂NR^{a14}R^{a15}, or —S(O)₂R^{a16}, wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R is optionally substituted with one or more halogens.

B46. The compound of any one of embodiments B30-B45, or a pharmaceutically acceptable salt thereof, wherein R¹ is

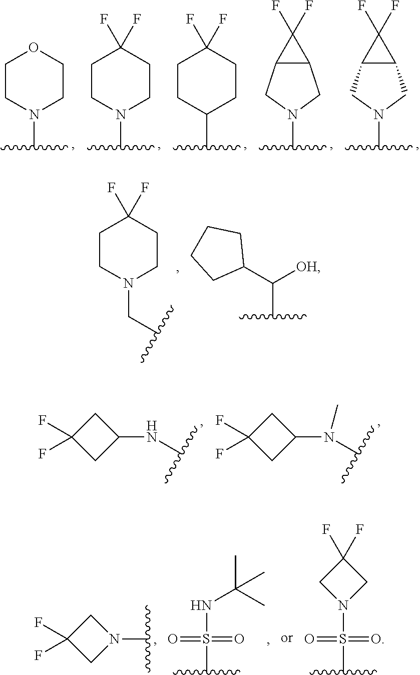

B47. The compound of any one of embodiments B30-B46 or a pharmaceutically acceptable salt thereof, wherein the ring

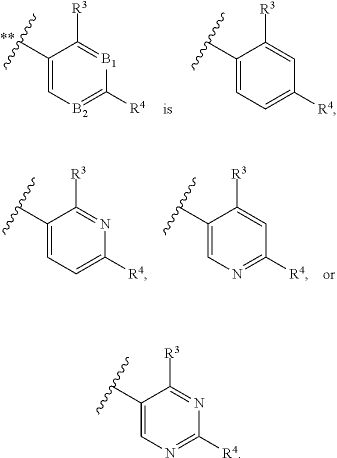

B48. The compound of any one of embodiments B30-B47, or a pharmaceutically acceptable salt thereof, wherein the ring

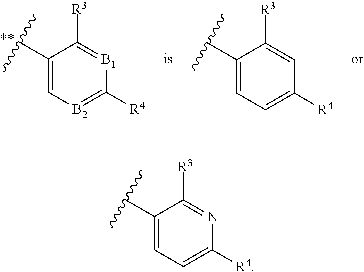

B49. The compound of any one of embodiments B30-B48, or a pharmaceutically acceptable salt thereof, wherein R³ is piperidinyl, wherein the piperidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, and
wherein the piperidinyl or the spirocyclic or fused bicyclic ring system formed by the C₃₋₁₀ cycloalkyl or 3- to 10-membered heterocycloalkyl with piperidinyl is optionally substituted with one or more substituents independently selected from the group consisting of C₁-C₃ alkyl and C₁-C₃ haloalkyl.

B50. The compound of any one of embodiments B30-B49, or a pharmaceutically acceptable salt thereof, wherein R³ is

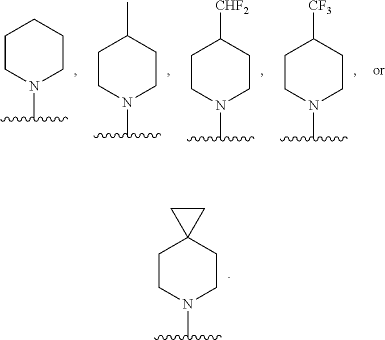

B51. The compound of any one of embodiments B30-B48, or a pharmaceutically acceptable salt thereof, wherein R³ is pyrrolidinyl, wherein the pyrrolidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the pyrrolidinyl, and
wherein the pyrrolidinyl or the spirocyclic or fused bicyclic ring system formed by the C₃₋₁₀ cycloalkyl or 3- to 10-membered heterocycloalkyl with pyrrolidinyl is optionally substituted with one or more substituents independently selected from the group consisting of C₁-C₃ alkyl and C₁-C₃ haloalkyl.

B52. The compound of any one of embodiments B30-B48 and B51, or a pharmaceutically acceptable salt thereof, wherein R³ is

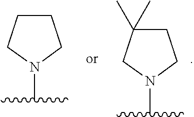

B53. The compound of any one of embodiments B30-B48, or a pharmaceutically acceptable salt thereof, wherein R³ is azepanyl, wherein the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the azepanyl, and
wherein the azepanyl or the spirocyclic or fused bicyclic ring system formed by the C₃₋₁₀ cycloalkyl or 3- to 10-membered heterocycloalkyl with azepanyl is optionally substituted with one or more substituents independently selected from the group consisting of C₁-C₃ alkyl and C₁-C₃ haloalkyl.

B54. The compound of any one of embodiments B30-B48 and B53, or a pharmaceutically acceptable salt thereof, wherein R³ is

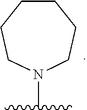 [SMILES: *N1CCCCCC1]

B55. The compound of any one of embodiments B30-B54, or a pharmaceutically acceptable salt thereof, wherein R⁴ is hydrogen, halo, or —NR^{c5}S(O)₂R^{c6}.

B56. The compound of any one of embodiments B30-B55, or a pharmaceutically acceptable salt thereof, wherein R⁴ is H, Br,

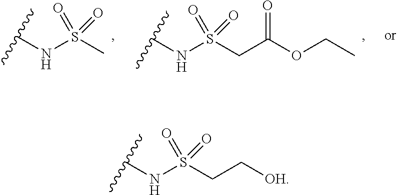

B57. The compound of any one of embodiments B30-B56, or a pharmaceutically acceptable salt thereof, wherein R⁴ is

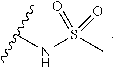 [SMILES: *NS(C)(=O)=O]

B58. The compound of embodiment B1, or a pharmaceutically acceptable salt thereof, wherein the compound is selected from the group consisting of compounds of Table 1. B59. A pharmaceutical composition comprising a compound of any one of embodiments B1-B58, or a pharmaceutically acceptable salt thereof, and a pharmaceutically acceptable carrier or excipient.

B60. A method of inhibiting KIF18A comprising contacting a cell with an effective amount of a compound of any one of embodiments B1-B58, or a pharmaceutically acceptable salt thereof, or a pharmaceutical composition of embodiment B59.

B61. A method of treating a disease or condition mediated by KIF18A in a subject in need thereof, comprising administering to the subject a therapeutically effective amount of a compound of any one of embodiments B1-B58, or a pharmaceutically acceptable salt thereof, or a pharmaceutical composition of embodiment B59.

B62. A method of treating cancer in a subject in need thereof, comprising administering to the subject a therapeutically effective amount of a compound of any one of embodiments B1-B58, or a pharmaceutically acceptable salt thereof, or a pharmaceutical composition of embodiment B59.

B63. The method of embodiment B62, wherein the cancer is selected from the group consisting of carcinomas, cancer of the anus, bladder, breast, colon, small intestine, appendix, kidney, renal pelvis, ureter, urothelium, liver, lung, pleura, esophagus, head and neck, nasopharynx, oropharynx, hypopharynx, oral cavity, larynx, biliary tract, gall-bladder, ovary, testicle, germ cell, uterus, pancreas, stomach, cervix, thyroid, prostate, salivary gland, or skin, hematopoietic tumors of lymphoid lineage, hematopoietic tumors of myeloid lineage, hematopoietic tumors of any lineage, myeloma, tumors of mesenchymal origin including sarcomas, tumors of the central and peripheral nervous system, tumor of neuroendocrine origin, tumor of endocrine origin, small cell tumors, tumors of unknown primary, other tumors comprising retinoblastoma, melanoma, seminoma, teratocarcinoma, osteosarcoma, and other cancer-related disorders that are a consequence of cancer presence or progression.

### EXAMPLES

The following examples are offered to illustrate but not to limit the compositions, uses, and methods provided herein. The compounds are prepared using the general methods described above.

### Abbreviations

BSA: bovine serum albumin
DMF: dimethyl formamide
ESI MS: electrospray mass spectrometry
HPLC: high-performance liquid chromatography
IC₅₀: 50% inhibitory concentration
Ms: methanesulfonyl
MsCl: methanesulfonyl chloride
NBS: N-bromosuccinimide
NCS: N-chlorosuccinimide
NMP: N-methylpyrrolidinone
NMR: nuclear magnetic resonance
PE: petroleum ether
TBAF: tetra-n-butylammonium fluoride
THF: tetrahydrofuran
TFA: trifluoroacetic acid

### Synthesis of Intermediates

### Synthesis of ethyl 4-bromo-2-fluorobenzimidate hydrochloride (I01.01)

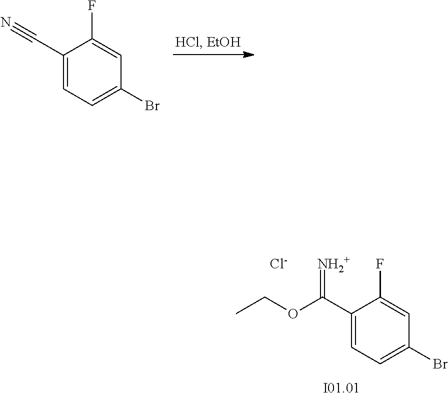

HCl gas was bubbled at 15 psi for 2 h through a 0° C. mixture of 4-bromo-4-fluoro-benzonitrile (1.0 g, 5.0 mmol), CH₂Cl₂ (20 mL), and EtOH (10 mL). The mixture was stirred for 2 h at 0° C., then 12 h at 20° C. and was concentrated to provide 1.2 g of 4-bromo-2-fluorobenzimidate hydrochloride (I01.01).

Imidate esters in Table 2 were prepared from the corresponding nitrile in the same manner as I01.01.

TABLE 2

| Name | Structure | Nitrile |
|---|---|---|
| I01.02 | 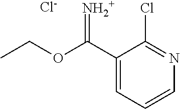 | 2-chloro-3-cyanopyridine |
| I01.03 | 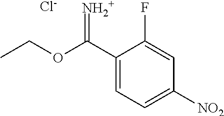 | 2-fluoro-4-nitro- benzonitrile |

### Synthesis of 6-morpholinopicolinohydrazide (I02.01)

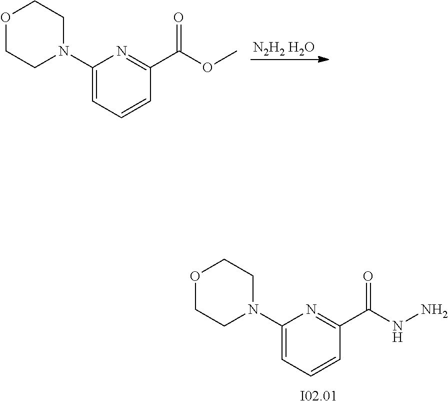

A mixture of methyl 6-morpholinopyridine-2-carboxylate (4.0 g, 18 mmol), EtOH (20 mL), and hydrazine hydrate (3.1 mL, 63 mmol) was stirred at 80° C. for 6 h. The mixture was cooled to 0° C. and allowed to stand for 1 h. The resulting precipitate was filtered, washed with EtOH (3×2 mL) and dried under vacuum to provide 3.0 g of 6-morpholinopicolinohydrazide (I02.01). ¹H NMR (DMSO-d⁶, 400 MHz) δ 9.65 (s, 1H), 7.75-7.60 (m, 1H), 7.28 (d, J=7.2 Hz, 1H), 6.97 (d, J=8.4 Hz, 1H), 4.49 (br s, 2H), 3.80-3.63 (m, 4H), 3.60-3.49 (m, 4H).

Compounds in Table 3 were prepared from the corresponding methyl ester in the same manner as I02.01.

TABLE 3

| Name | Structure |
|---|---|
| I02.02 | 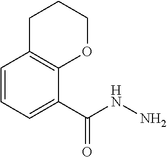 [SMILES: NNC(=O)c1cccc2c1OCCC2] |
| I02.03 | 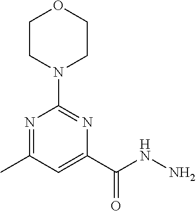 [SMILES: Cc1cc(C(=O)NN)nc(N2CCOCC2)n1] |
| I02.04 | 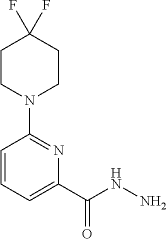 [SMILES: NNC(=O)c1cccc(N2CCC(F)(F)CC2)n1] |

### Synthesis of 2-bromo-1-(6-(4,4-difluoropiperidin-1-yl)pyridin-2-yl)ethan-1-one (I03.02)

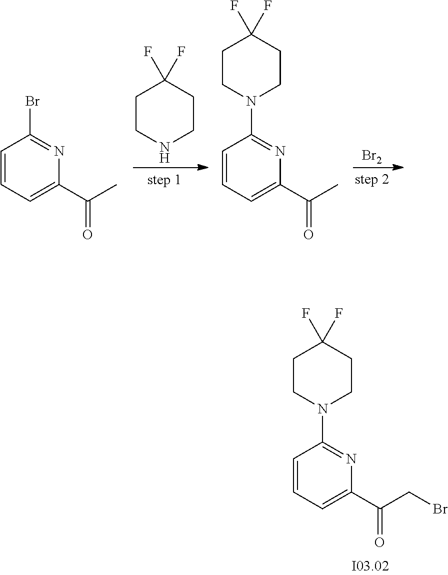

Step 1. A mixture of 1-(6-bromo-2-pyridyl)ethanone (2.0 g, 10 mmol), DMF (20 mL), K₂CO₃ (2.7 g, 20 mmol), and 4,4-difluoropiperidine hydrochloride (1.6 g, 10 mmol) was stirred at 130° C. for 12 h. The mixture was poured into 30 mL of H₂O and extracted with EtOAc (2×20 mL). The extracts were combined, washed with brine (10 mL), dried over Na₂SO₄, filtered, concentrated, and purified by silica chromatography (0-100% EtOAc in PE) to provide 1.6 g of 1-(6-(4,4-difluoropiperidin-1-yl)pyridin-2-yl)ethan-1-one (I03.01).

Step 2. Br₂ (0.31 mL, 6.0 mmol) was added to 103.01 (1.6 g, 6.7 mmol), HBr (12 mL), and dioxane (6 mL). The mixture was stirred at 60° C. for 12 h. Aqueous NaHSO₃ (10 mL) was added and Na₂CO₃ added to bring the pH to 7 and the mixture combined with 30 mL of water and extracted with EtOAc (2×30 mL). The combined extracts were washed with brine, dried over Na₂SO₄, filtered, concentrated, and purified by silica chromatography (0-100% EtOAc in PE) followed by reverse-phase HPLC (C18, 30-70% MeCN in H₂O [0.1% formic acid]) to provide 0.54 g of 2-bromo-1-(6-(4,4-difluoropiperidin-1-yl)pyridin-2-yl)ethan-1-one (I03.02).

Compounds in Table 4 were prepared in the same manner as 103.02 from the corresponding ketone.

TABLE 4

| Name | Structure |
|---|---|
| I03.03 | 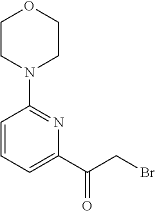 [SMILES: O=C(CBr)c1cccc(N2CCOCC2)n1] |
| I03.10 | 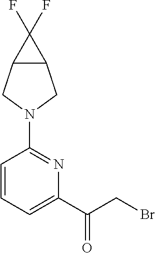 [SMILES: O=C(CBr)c1cccc(N2CC3C(C2)C3(F)F)n1] |

### Synthesis of 3-(2-bromoacetyl)-N-(tert-butyl)benzenesulfonamide (I03.06)

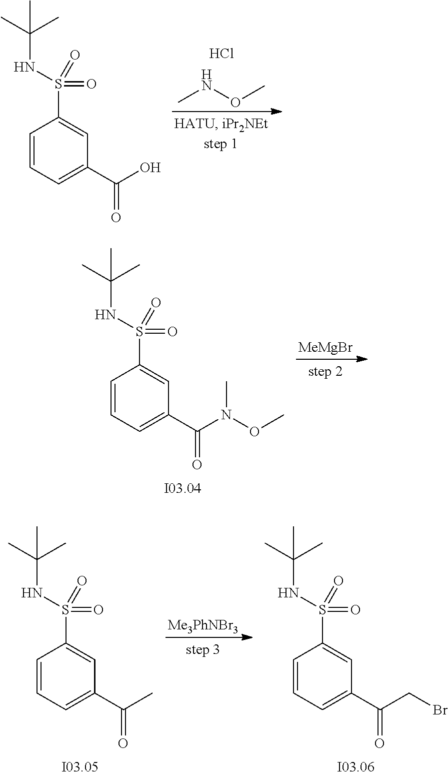

Step 1. A mixture of 3-(tert-butylsulfamoyl)benzoic acid (2.0 g, 7.8 mmol), DMF (20 mL), HATU (4.5 g, 12 mmol), and iPr₂NEt (5.4 mL, 31 mmol) was stirred at 20° C. for 0.5 h. N-methoxymethanamine hydrochloride (1.1 g, 11 mmol) was added and the mixture was stirred at 20° C. for 12 h and combined with EtOAc (30 mL) and washed with H₂O (15 mL×2) and brine (15 mL), dried over Na₂SO₄, filtered, concentrated and purified by silica chromatography (0-50% EtOAc/PE) to provide 3-(tert-butylsulfamoyl)-N-methoxy-N-methyl-benzamide (I03.04, 2.1 g). ¹H NMR: (CDCl₃, 400 MHz) δ ppm 8.24 (s, 1H), 7.99 (dd, J=7.6, 1.25 Hz, 1H), 7.88 (d, J=7.6 Hz, 1H), 7.55 (t, J=7.6 Hz, 1H), 4.65 (br s, 1H), 3.54 (s, 3H), 3.39 (s, 3H), 1.24 (s, 9H).

Step 2. To a 0° C. mixture of 103.04 (1.0 g, 3.3 mmol), THF (15 mL) was added MeMgBr (3 M, 3.3 mL). The mixture was stirred at 20° C. for 5 h, and saturated NH₄Cl (20 mL) was added at 0° C. EtOAc (40 mL) was added, and the organic phase separated, washed with brine (15 mL), dried over Na₂SO₄, filtered, concentrated, and purified by silica chromatography (0-30% EtOAc/PE) to provide 3-acetyl-N-tert-butyl-benzenesulfonamide (I03.05, 0.75 g). ¹H NMR: (CDCl₃, 400 MHz) δ ppm 8.51-8.41 (m, 1H), 8.18-8.04 (m, 2H), 7.67-7.56 (m, 1H), 4.72 (s, 1H) 2.66 (s, 3H) 1.26 (s, 9H).

Step 3. To a mixture of 103.05 (0.5 g, 2.0 mmol) and THF (15 mL) was added phenyltrimethylammonium perbromide (0.77 g, 2.1 mmol) and the mixture was stirred at 20° C. for 12 h. The mixture was filtered, and the filtrate was concentrated and purified by silica chromatography (0-40% EtOAc/PE) to provide 3-(2-bromoacetyl)-N-tert-butyl-benzenesulfonamide (I03.06, 0.55 g). ¹H NMR (CDCl₃, 400 MHz) δ ppm 8.49 (s, 1H) 8.16 (br t, J=9.19 Hz, 2H) 7.66 (t, J=7.82 Hz, 1H) 4.85 (br s, 1H) 4.47 (s, 2H) 1.26 (s, 9H).

### Synthesis of 2-bromo-1-[6-[(3,3-difluorocyclobutyl)amino]-2-pyridyl]ethanone (I03.09)

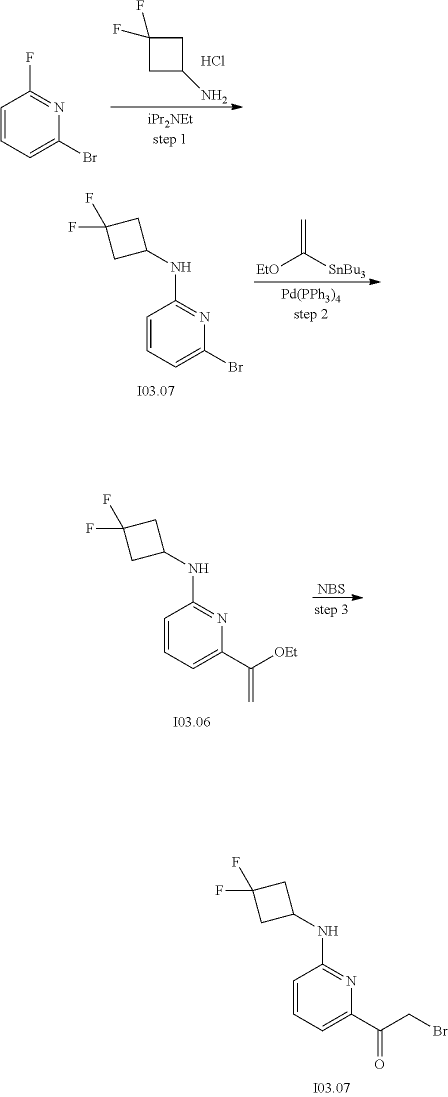

Step 1. A mixture of 3,3-difluorocyclobutanamine hydrochloride (4.9 g, 34 mmol), iPrOH (15 mL), was iPr₂NEt (5.9 mL, 34 mmol), and 2-bromo-6-fluoro-pyridine (2.0 g, 11 mmol) was stirred at 90° C. for 12 h, the was poured into water (10 mL) and extracted with EtOAc (2×10 mL). The organic phase was washed with brine (10 mL), dried over Na₂SO₄, concentrated, and purified by silica chromatography (10-50% EtOAc in PE) to provide 6-bromo-N-(3,3-difluorocyclobutyl)pyridin-2-amine (I03.07, 1.6 g).

Step 2. A mixture of 103.07 (0.60 g, 2.3 mmol) and tributyl(1-ethoxyvinyl)stannane (1.2 mL, 3.4 mmol) in dioxane (10 mL), CsF (0.69 mg, 4.6 mmol), Pd(PPh₃)₄ (0.13 g, 0.11 mmol) was stirred at 130° C. for 2 h. To the reaction mixture was added a solution of KF (0.4 g) in water (50 mL) and the mixture was stirred at 20° C. for 0.5 h. The mixture was diluted with EtOAc (10 mL), washed with brine (10 mL×2), dried over Na₂SO₄, filtered, concentrated, and purified by silica chromatography (10-50% EtOAc in PE) to provide N-(3,3-difluorocyclobutyl)-6-(1-ethoxyvinyl)pyridin-2-amine (I03.08, 0.55 g).

Step 3. To a mixture of 103.08 (0.55 g, 2.2 mmol), THF (5 mL), and H₂O (2 mL) was added NBS (0.31 g, 1.7 mmol). The mixture was stirred at 20° C. for 2 h, poured into water (10 mL), and extracted with EtOAc (2×10 mL). The organic phase was washed with brine (10 mL), dried over Na₂SO₄, concentrated, and purified by silica chromatography (10-50% EtOAc in PE) to provide 2-bromo-1-[6-[(3,3-difluorocyclobutyl)amino]-2-pyridyl]ethanone (I03.09, 0.25 g).

### Synthesis of 2-bromo-1-(6-(4,4-difluoropiperidin-1-yl)-4-methylpyridin-2-yl)ethan-1-one (I03.13)

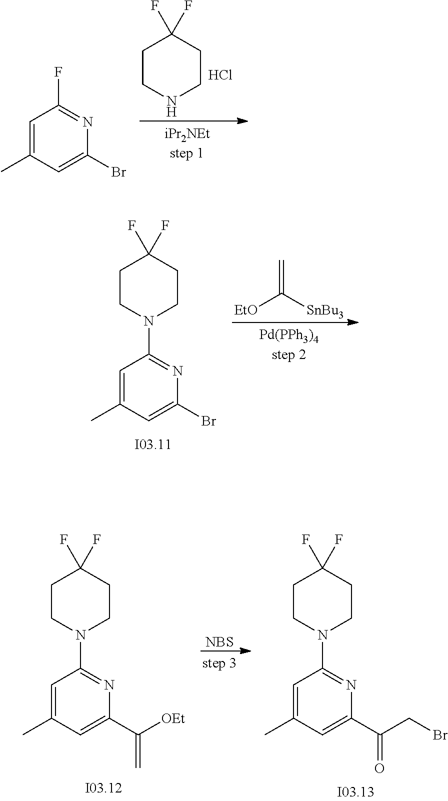

I03.13 was prepared in the same manner as I03.09 by replacing 3,3-difluorocyclobutanamine hydrochloride with 4,4-difluoropiperidine hydrochloride and 2-bromo-6-fluoro-pyridine with 2-bromo-4-methyl-6-fluoro-pyridine.

### Synthesis of 2-bromo-1-(2-(4,4-difluoropiperidin-1-yl)-6-methylpyrimidin-4-yl)ethan-1-one (I03.16)

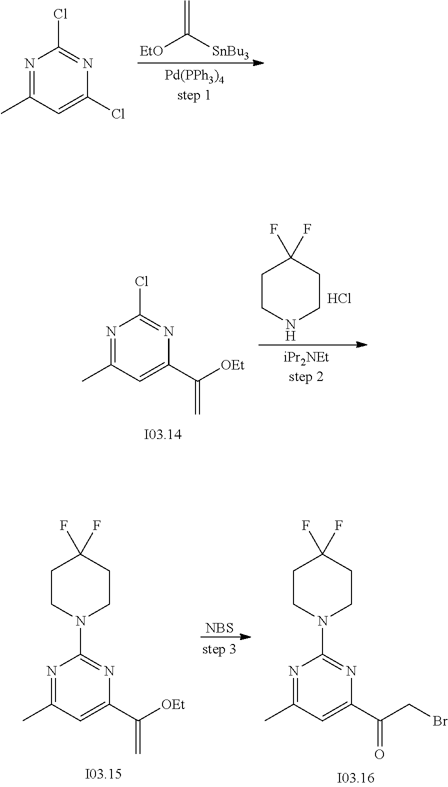

I03.16 was prepared in the same manner as I03.09 by replacing 2-bromo-6-fluoro-pyridine with 2,6-dichloro-4-methylpyrimidine, and by changing the order of reactions as indicated in the above scheme.

### Synthesis of 4-nitro-2-(6-azaspiro[2.5]octan-6-yl)benzimidamide (I04.02)

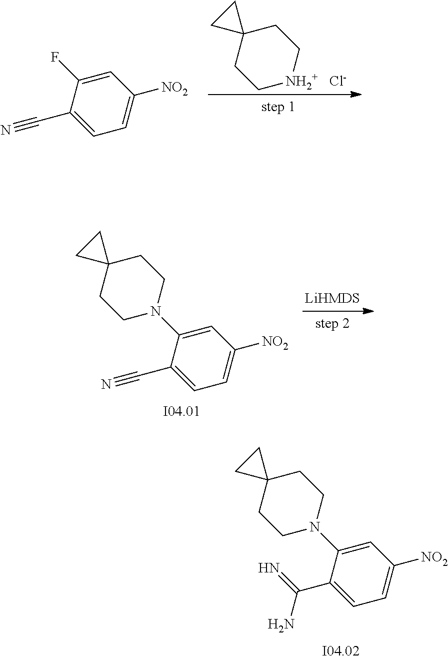 [SMILES: N#Cc1ccc([N+](=O)[O-])cc1F ; C1CC2(CC[NH2+]1)CC2 ; [Cl-] ; N#Cc1ccc([N+](=O)[O-])cc1N1CCC2(CC1)CC2 ; N=C(N)c1ccc([N+](=O)[O-])cc1N1CCC2(CC1)CC2]

Step 1. A mixture of 2-fluoro-4-nitro-benzonitrile (5.0 g, 30 mmol), DMF (30 mL), K₂CO₃ (8.3 g, 60 mmol), and 6-azaspiro[2.5]octane hydrochloride (4.4 g, 30 mmol) was stirred at 120° C. for 12 h. The mixture was poured into H₂O (80 mL), extracted with EtOAc (2×100 mL), and the combined extracts were washed with brine (50 mL), dried over Na₂SO₄, filtered, concentrated, and triturated with MeOH (25 mL) for 0.5 h to provide 6.8 g of 4-nitro-24(6-azaspiro[2.5]octan-6-yl)benzonitrile (I04.01). ¹H NMR (DMSO-d⁶, 400 MHz) ppm δ 8.04-7.92 (in, 1H), 7.86-7.73 (m, 2H), 3.36-3.28 (m, 4H), 1.58-1.47 (m, 4H), 0.37 (s, 4H).

Step 2. To a mixture of 104.01 (6.5 g, 25 mmol) and THF (60 mL) was added 1 M LiHMDS (130 mL, 130 mmol). The mixture was stirred at 20° C. for 12 h, and 2 M HCl (40 mL) was added at a rate to maintain the internal temperature below 30° C. The mixture was partially concentrated and then it was washed with EtOAc, and the pH adjusted to 8 by the slow addition of saturated NaHCO₃(30 mL). The resulting organic phase was collected and concentrated to provide 1.7 g of 4-nitro-2-(6-azaspiro[2.5]octan-6-yl)benzimidamide (I04.02).

### Synthesis of 4-bromo-2-(6-azaspiro[2.5]octan-6-yl)benzimidamide (I04.05)

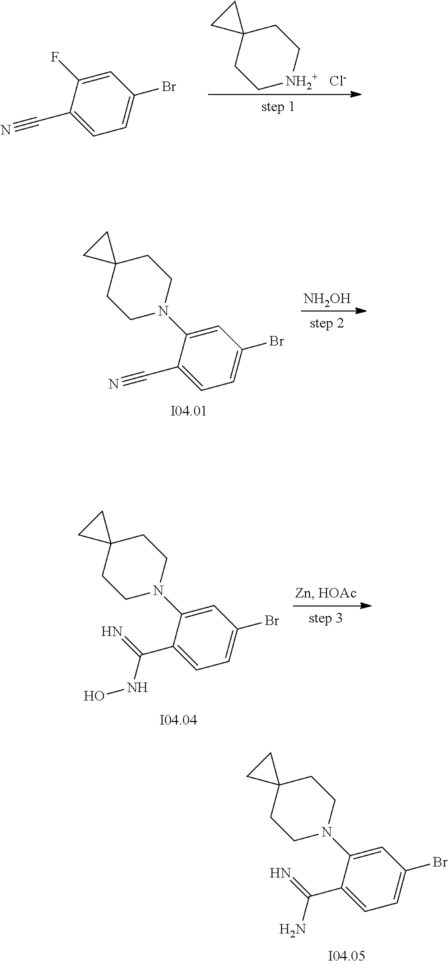

Step 1. A mixture of 6-azaspiro[2.5]octane hydrochloride (2.7 g, 18 mmol), iPr₂NEt (13 mL, 75 mmol), DMSO (30 mL), and 4-bromo-2-fluoro-benzonitrile (3.0 g, 15 mmol) was stirred at 140° C. for 12, poured into water (100 mL), and extracted with EtOAc (2×10 mL). The organic phase was washed with brine (10 mL), dried over Na₂SO₄, concentrated, and purified by flash silica chromatography (0-20% EtOAc in PE) to provide 2-(6-azaspiro[2.5]octan-6-yl)-4-bromo-benzonitrile (I04.03, 4.0 g).

Step 2. A mixture of 104.03 (4.0 g, 14 mmol), hydroxylamine (50% in H₂O, 1.8 g, 28 mmol), EtOH (40 mL) was stirred at 100° C. for 12 h, poured to water (150 mL), and extracted with EtOAc (2×150 mL). The organic phase was washed with brine (10 mL), dried over Na₂SO₄, concentrated, and triturated with CH₂Cl₂ (20 mL) at 20° C. for 0.5 h to provide 2-(6-azaspiro[2.5]octan-6-yl)-4-bromo-N-hydroxy-benzamidine (I04.04, 3.0 g).

Step 3. A degassed mixture of 104.04 (3.0 g, 9.3 mmol), Zn (6.1 g, 93 mmol), HOAc (30 mL) was degassed was stirred at 80° C. for 0.5 hour under an N₂ atmosphere. The mixture was cooled, filtered through a Celite pad, and was poured into water (50 mL), extracted with EtOAc (2×50 mL). The extracts were washed with brine (10 mL), dried over Na₂SO₄, concentrated, and triturated with 10:1 EtOAc/PE at 20° C. for 30 min to provide 2-(6-azaspiro[2.5]octan-6-yl)-4-bromo-benzamidine (I04.05, 2.1 g).

### Synthesis of 4-bromo-2-(6-azaspiro[2.5]octan-6-yl)benzaldehyde (I04.06)

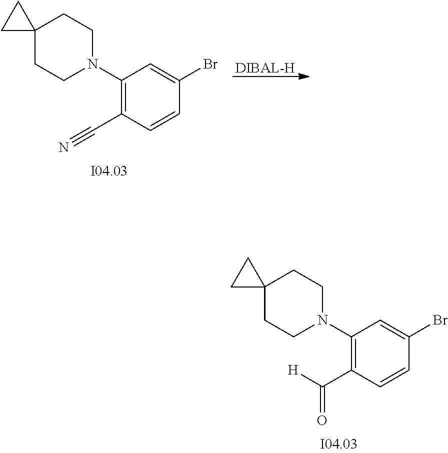 [SMILES: N#Cc1ccc(Br)cc1N1CCC2(CC1)CC2 ; "DIBAL-H" ; O=Cc1ccc(Br)cc1N1CCC2(CC1)CC2]

To a mixture of 104.03 (2.2 g, 7.6 mmol) and CH₂CL₂ (30 mL) was added DIBAL-H (1 M, 11 mL) at −5° C., then the mixture was stirred at −5° C. for 2 h under a N₂ atmosphere. Aqueous HCl (2 N, 10 mL) was added slowly into the mixture, and then the mixture poured into saturated Na₂CO₃ (50 mL). The resulting mixture was extracted with CH₂Cl₂ (2×50 mL), and the combined extracts were washed with brine (30 mL), dried over Na₂SO₄, concentrated, and purified by silica chromatography (0-50% EtOAc in PE) to provide 4-bromo-2-(6-azaspiro[2.5]octan-6-yl)benzaldehyde (I04.06, 1.5 g, 91% purity).

### Synthesis of 4-bromo-2-(4-methyl-1-piperidyl)benzaldehyde (I04.08)

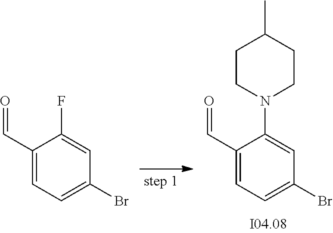 [SMILES: O=Cc1ccc(Br)cc1F ; CC1CCN(c2cc(Br)ccc2C=O)CC1]

A mixture of 4-bromo-2-fluoro-benzaldehyde (2.0 g, 10 mmol), 4-methylpiperidine (1.0 mL, 10 mmol), DMF (40 mL), K₂CO₃ (4 g, 30 mmol) was stirred at 100° C. for 12 h, cooled, combined with H₂O (100 mL), and extracted with EtOAc (2×50 mL). The extracts were combined, washed with brine (20 mL), dried over Na₂SO₄, filtered, concentrated, and purified by silica chromatography (5-50% EtOAc in PE) to provide 4-bromo-2-(4-methyl-1-piperidyl)benzaldehyde (I04.08, 2.3 g).

### Synthesis of 4-iodospiro[benzo[d][1,3]dioxole-2,1′-cyclohexane] (I04.07)

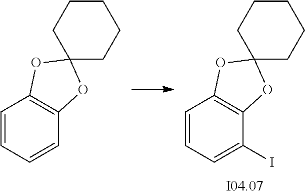 [SMILES: c1ccc2c(c1)OC1(CCCCC1)O2 ; Ic1cccc2c1OC1(CCCCC1)O2]

To a mixture of spiro[1,3-benzodioxole-2,1′-cyclohexane] (2.0 g, 11 mmol) and THF (20 mL) under Ar₂ at −75° C. was added sBuLi (1.3 M, 12 mL, 16 mmol). The mixture was stirred for 2 h under Ar₂, then I₂ (2.7 g, 11 mmol) was added at −75° C. The mixture was stirred for 2 h at −75° C., warmed, and poured into H₂O (30 mL) and the resulting mixture was extracted with EtOAc (2×30.0 mL). The extracts were combined, washed with brine (10 mL), dried over Na₂SO₄, filtered, concentrated, and purified by silica chromatography (0-100% EtOAc in PE) to provide 4-iodospiro[1,3-benzodioxole-2,1′-cyclohexane] (I04.07, 0.73 mg). ¹H NMR (400 MHz, DMSO-d⁶) δ ppm 7.03-7.13 (m, 1H) 6.76-6.89 (m, 1H) 6.50-6.64 (m, 1H) 1.80-1.97 (m, 4H) 1.56-1.72 (m, 4H) 1.39-1.50 (m, 2H)

Alkyne Synthesis Method I05A:

### Synthesis of 2-(4,4-difluoropiperidin-1-yl)-6-ethynylpyridine (I05.03)

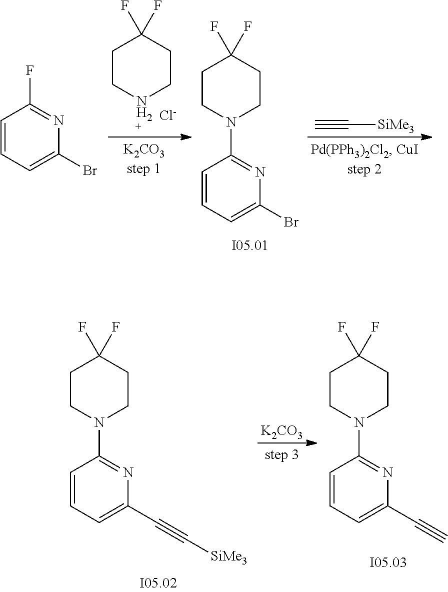

Step 1. A degassed mixture of 2-bromo-6-fluoropyridine (2.0 g, 11 mmol), 4,4-difluoropiperidine hydrochloride (2.7 g, 17 mmol), K₂CO₃ (4.7 g, 34 mmol), and DMF (20 mL) was stirred at 130° C. for 12 h under N₂. The mixture was cooled, concentrated, and purified by silica chromatography (0-30% EtOAc in PE) to provide 2.0 g of 2-bromo-6-(4,4-difluoropiperidin-1-yl)pyridine (I05.01).

Step 2. A degassed mixture of I05.01 (1.1 g, 4.0 mmol), ethynyltrimethylsilane (1.7 mL, 12 mmol), Pd(PPh₃)₂Cl₂ (0.28 g, 0.40 mmol), CuI (75 mg, 0.40 mmol), Et₃N (1.7 mL, 12 mmol), and THF (15 mL) was stirred at 55° C. for 12 h under N₂. The mixture was concentrated and purified by silica chromatography (0-30% EtOAc in PE) to provide 0.50 g of 2-(4,4-difluoropiperidin-1-yl)-6-((trimethylsilyl)ethynyl)pyridine (I05.02).

Step 3. A mixture of I05.02 (0.46 g, 1.6 mmol), K₂CO₃ (0.43 g, 3.1 mmol), and MeOH was stirred at 20° C. for 12 h. The mixture was concentrated and purified by silica chromatography (0-30% EtOAc in PE) to provide 0.25 g of 2-(4,4-difluoropiperidin-1-yl)-6-ethynylpyridine (I05.03).

### Synthesis of 2-(4,4-difluoropiperidin-1-yl)-4-ethynylthiazole (R-038)

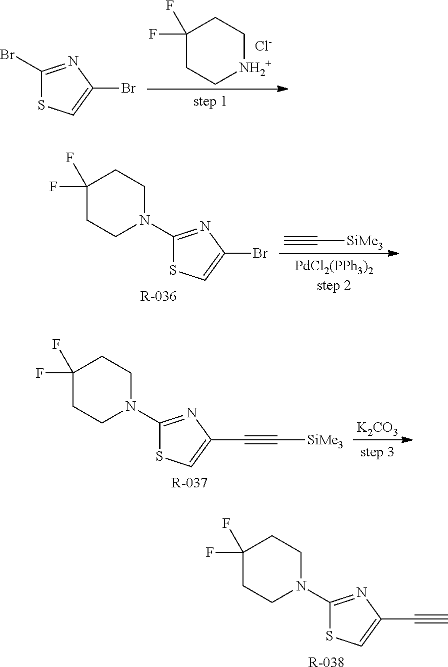

Step 1. A mixture of 2,4-dibromothiazole (1.0 g, 4.1 mmol), 4,4-difluoropiperidine hydrochloride (1.3 g, 8.2 mmol), DMF (10 mL), and Et₃N (2.3 g, 17 mmol) was stirred at 80° C. for 16 h. The mixture was combined with H₂O (50 mL) and extracted with EtOAc (30 mL×3). The combined extracts were washed with brine (30 mL×3), dried over Na₂SO₄, filtered, concentrated, and purified by silica chromatography (0-5% EtOH in PE) to provide 4-bromo-2-(4,4-difluoro-1-piperidyl)thiazole (R-036; 1.0 g).

Steps 2-3. 2-(4,4-difluoropiperidin-1-yl)-4-ethynylthiazole (R-038) was prepared in two steps as described for step 2 and step 3 of Alkyne Preparation Method I05A by substituting R-036 for I05.01.

### Synthesis of 2-(cyclopentyloxy)-6-ethynylpyrazine (I05.66)

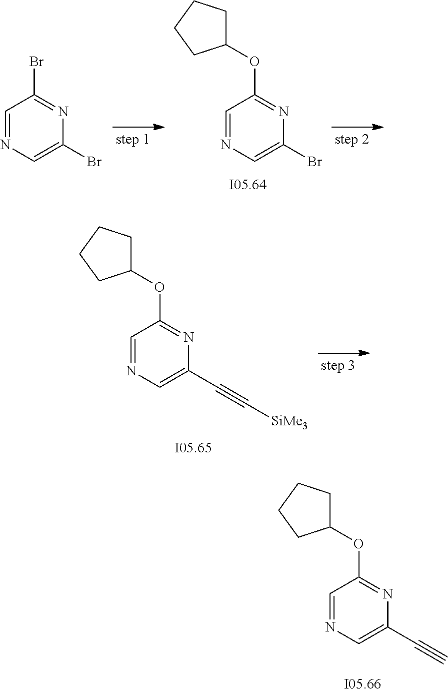 [SMILES: Brc1cncc(Br)n1 ; Brc1cncc(OC2CCCC2)n1 ; C[Si](C)(C)C#Cc1cncc(OC2CCCC2)n1 ; C#Cc1cncc(OC2CCCC2)n1]

Step 1. A mixture of 2,6-dibromopyrazine (1.5 g, 6.3 mmol), cyclopentanol (0.57 mL, 6.3 mmol), DMF (20 mL), Cs₂CO₃ (4.1 g, 13 mmol) was stirred at 100° C. for 6 h, then poured into H₂O (50 mL) and extracted with EtOAc (2×25 mL). The extracts were combined, washed with brine (10 mL), dried over Na₂SO₄, concentrated, and purified by silica chromatography (10-50% EtOAc in PE) to provide 2-bromo-6-(cyclopentoxy) pyrazine (I05.64, 0.59 g).

Steps 2-3. 2-(Cyclopentyloxy)-6-ethynylpyrazine (I05.66) was prepared in two steps as described for step 2 and step 3 of Alkyne Preparation Method I05A by substituting I05.64 for 105.01.

Alkyne Synthesis Method I05B:

### Synthesis of 1-((3-ethynylphenyl)sulfonyl)-3,3-difluoroazetidine (I05.05)

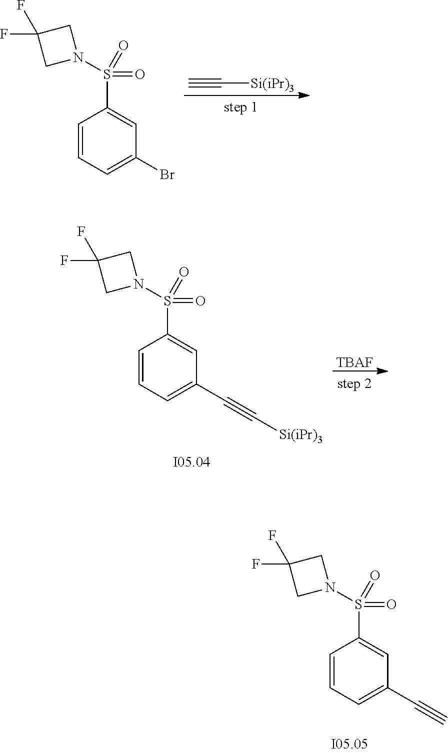

Step 1. A mixture of 1-(3-bromophenyl)sulfonyl-3,3-difluoro-azetidine (0.87 g, 2.8 mmol), MeCN (3 mL), Xantphos Pd G4 (0.27 g, 0.28 mmol), Cs₂CO₃ (2.7 g, 8.4 mmol), ethynyl(triisopropyl)silane (3.1 mL, 14 mmol), CuI (53 mg, 0.28 mmol) was stirred at 100° C. for 12 h. The mixture was poured into H₂O (30 mL) and extracted with EtOAc (2×30 mL). The extracts were washed with brine (10 mL), dried over Na₂SO₄, and concentrated to provide 2-[3-(3,3-difluoroazetidin-1-yl)sulfonylphenyl]ethynyl-triisopropyl-silane (I05.04, 3.2 g).

Step 2. A mixture of I05.04 (1.2 g, 2.2 mmol), THF (10 mL), and TBAF (1 M, 11 mL, 11 mmol) was stirred for 2 h at 25° C. The mixture was poured into H₂O (30 mL), extracted with EtOAc (30 mL×3), and the combined extracts were washed with brine (30 mL×2), dried over Na₂SO₄, filtered, and concentrated. A separate residue was prepared in the same manner from 0.2 g of I05.04. The combined residues were purified by silica chromatography (5-10% EtOAc in PE) to provide 1-(3-ethynylphenyl)sulfonyl-3,3-difluoro-azetidine (I05.05, 0.37 g).

Alkyne Synthesis Method I05C:

### Synthesis of 2-(4,4-difluoro-1-piperidyl)-6-ethynyl-4-methyl-pyridine (I05.08)

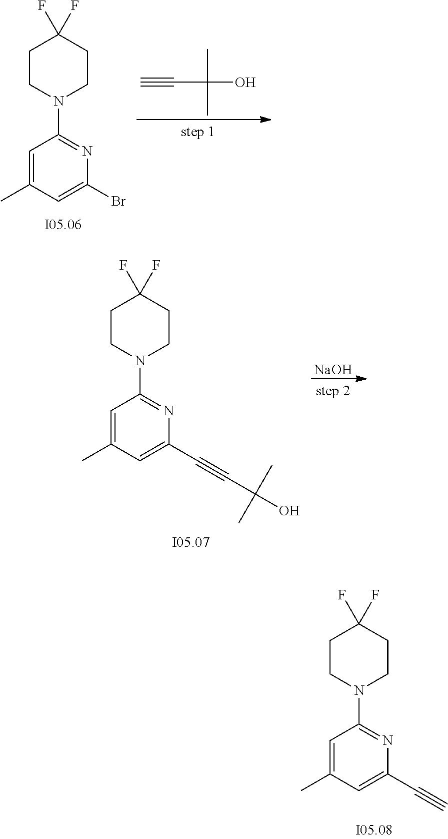

Step 1. 2-bromo-6-(4,4-difluoro-1-piperidyl)-4-methyl-pyridine (I05.06) was prepared from 2-bromo-6-fluoro-4-methylpyridine in the same manner as described I05.01. A degassed mixture of I05.06 (1.0 g, 3.4 mmol), 2-methylbut-3-yn-2-ol (2.0 mL, 21 mmol), CuI (65 mg, 0.34 μmol), Et₃N (1.4 mL, 10 mmol), Pd(PPh₃)₂Cl₂ (0.24 g, 0.34 mmol), and DMF (10 mL) was stirred at 140° C. for 1.2 h in a microwave reactor. The mixture was concentrated and purified by silica chromatography (0-30% EtOAc in PE) to provide 4-[6-(4,4-difluoro-1-piperidyl)-4-methyl-2-pyridyl]-2-methyl-but-3-yn-2-ol (I05.07, 0.50 g).

Step 2. To a mixture of I05.07 (0.50 g, 1.7 mmol) and toluene (1 mL) was added NaOH (0.10 g, 2.5 mmol). The mixture was stirred at 110° C. for 12 h, then was concentrated, diluted with water (50 mL), and extracted with EtOAc (50 mL×3). The combined organic layers were washed with brine (10 mL), dried over Na₂SO₄, filtered, and concentrated to provide 2-(4,4-difluoro-1-piperidyl)-6-ethynyl-4-methyl-pyridine (I05.08, 0.40 mg).

### Synthesis of 1-(4,4-difluorocyclohexyl)-3-ethynyl-1H-pyrazole (I05.24)

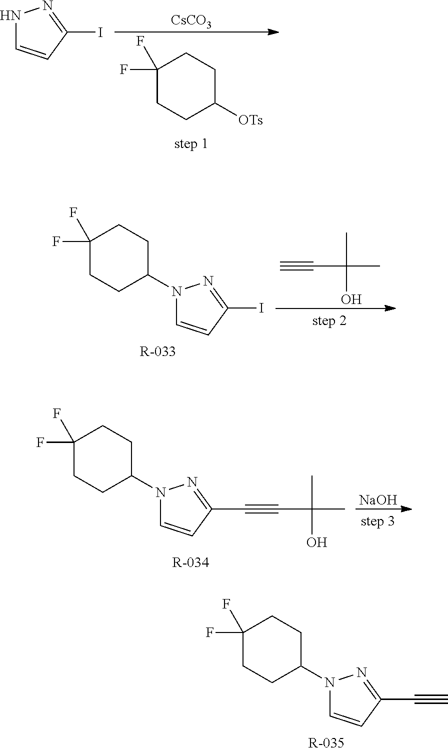

Step 1. A mixture of 3-iodo-1H-pyrazole (2.0 g, 10 mmol), DMF (20 mL), Cs₂CO₃ (10 g, 31 mmol), and (4,4-difluorocyclohexyl)-4-methylbenzenesulfonate (4.5 g, 16 mmol) was stirred at 90° C. for 12 h. The mixture was concentrated, combined with H₂O (50 mL) and extracted with EtOAc (50 mL×3). The combined extracts were washed with brine (30 mL), dried over Na₂SO₄, filtered, concentrated, and purified by silica chromatography (5-25% EtOAc in PE) to provide 1-(4,4-difluorocyclohexyl)-3-iodo-pyrazole (R-033, 2.0 g).

Steps 2-3 were run using the Alkyne Synthesis Method I05C with R-033 using in place of I05.06 to provide I05.24.

Alkyne Synthesis Method I05D:

### Synthesis of 2-(4,4-difluoropiperidin-1-yl)-4-ethynyl-6-methylpyrimidine (I05.11)

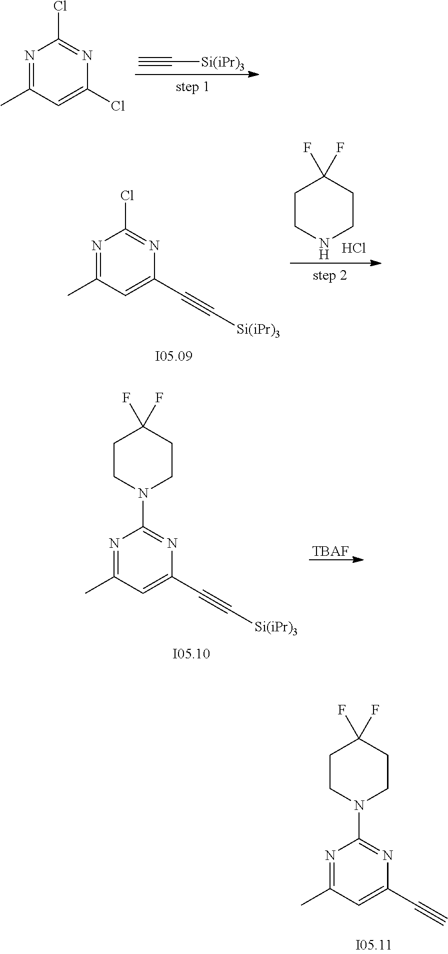

Step 1. A mixture of 2,4-dichloro-6-methyl-pyrimidine (2.0 g, 12 mmol) and ethynyl(triisopropyl)silane (8.3 mL, 37 mmol), THF (20 mL), added Pd(PPh₃)₂Cl₂ (0.43 g, 0.61 mmol), CuI (0.23 mg, 1.2 mmol), and Et₃N (5.1 mL, 37 mmol) was stirred at 50° C. for 12 h. The mixture was poured into H₂O (10 mL) and extracted with EtOAc (2×10 mL). The extracts were washed with brine (10 mL), dried over Na₂SO₄, concentrated, and purified by silica chromatography (0-10% EtOAc in PE) to provide 2-(2-chloro-6-methyl-pyrimidin-4-yl)ethynyl-triisopropyl-silane (I05.09, 1.2 g).

Step 2. A mixture of 4,4-difluoropiperidine (0.57 g, 4.7 mmol), DMF (5 mL), iPr₂NEt (2.0 mL, 12 mmol), and I05.09 (1.2 g, 3.9 mmol) was stirred at 120° C. for 1 h. The mixture was poured into water (10 mL) and extracted with EtOAc (2×10 mL). The combined extracts were washed with brine (10 mL), dried over Na₂SO₄, filtered, concentrated, and purified by silica chromatography (0-100% EtOAc in PE) to provide 2-[2-(4,4-difluoro-1-piperidyl)-6-methyl-pyrimidin-4-yl]ethynyl-triisopropyl-silane (I05.10, 1.5 g).

Step 3. A mixture of I05.10 (1.5 g, 3.8 mmol), THF (5 mL), and TBAF (1 M, 19 mL, 19 mmol) was stirred at 20° C. for 4 h. The mixture was poured into H₂O (10 mL) and extracted with EtOAc (2×10 mL). The combined extracts were washed with brine (10 mL), dried over Na₂SO₄, concentrated, and purified by silica chromatography (0-10% EtOAc in PE) to provide 2-(4,4-difluoro-1-piperidyl)-4-ethynyl-6-methyl-pyrimidine (I05.11, 0.60 g).

### Synthesis of 2-(cyclopentyloxy)-4-ethynyl-6-methylpyrimidine (I05.37)

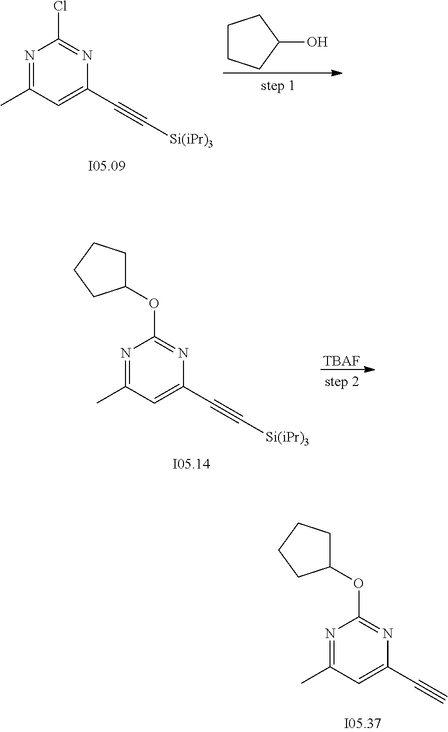

Step 1. A degassed mixture of I05.09 (1.0 g, 4.0 mmol), cyclopentanol (0.96 g, 11 mmol), dioxane (25 mL), and Cs₂CO₃ (3.0 g, 9.2 mmol) was stirred at 100° C. for 12 h under an N₂ atmosphere. The mixture was diluted with EtOAc (40 mL) and filtered. The filtrate was washed with H₂O (20 mL), brine (20 mL), dried over Na₂SO₄, filtered, concentrated, and purified by silica chromatography (0-10% EtOAc in PE) to provide 2-(cyclopentyloxy)-4-methyl-6-((triisopropylsilyl)ethynyl)pyrimidine (I05.14, 1.0 g).

Step 2. 2-(Cyclopentyloxy)-4-ethynyl-6-methylpyrimidine (I05.37) was prepared from I05.14 by treatment with TBAF in the manner described in step 3 of the synthesis for 105.11.

Alkyne Synthesis Method I05E:

### Synthesis of 3-(4,4-difluoropiperidin-1-yl)-5-ethynyl-2-methylpyrazine

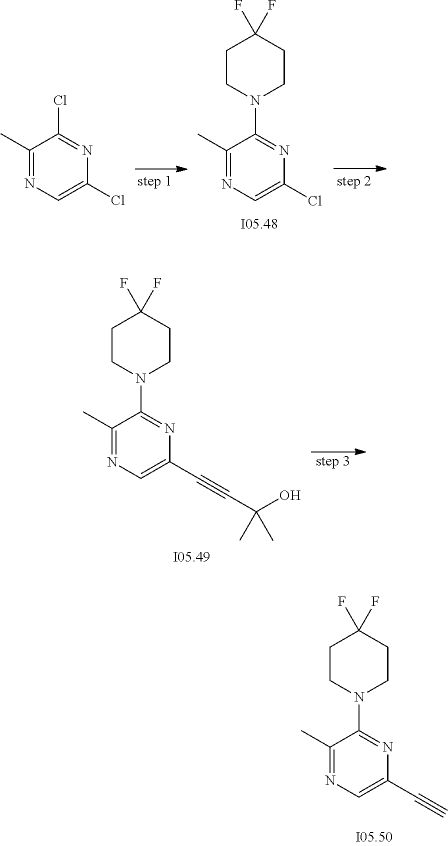

Step 1. A mixture of 3,5-dichloro-2-methyl-pyrazine (2.0 g, 12 mmol), 4,4-difluoropiperidine hydrochloride (1.9 g, 12 mmol), DMSO (40 mL), K₂CO₃ (5.1 g, 37 mmol) was stirred at 100° C. for 12 h, then was cooled and poured into H₂O (20 mL) and the resulting mixture was extracted with EtOAc (2×25 mL). The combined extracts were washed with brine (10 mL), dried over Na₂SO₄, filtered, concentrated, and purified by silica chromatography (10-50% EtOAc in PE) to provide 5-chloro-3-(4,4-difluoro-1-piperidyl)-2-methyl-pyrazine (I05.48, 1.0 g).

Step 2. A degassed mixture of I05.48 (0.81 g, 3.3 mmol), 2-methylbut-3-yn-2-ol (0.96 mL, 9.8 mmol), CuI (62 mg, 0.33 mmol), Pd(dppf)Cl₂ (0.24 g, 0.33 mmol), KF (0.38 g, 6.5 mmol), PPh₃ (86 mg, 0.33 mmol), iPr₂NEt (1.1 mL, 6.5 mmol), DMF (16 mL) was stirred at 120° C. for 2 h under an N₂ atmosphere. The reaction mixture was poured into water (20 mL), extracted with EtOAc (2×15 mL) and the extracts were combined, washed with brine (10 mL), dried over Na₂SO₄, concentrated, and purified by silica chromatography (10-100% EtOAc in PE) to provide 4-[6-(4,4-difluoro-1-piperidyl)-5-methyl-pyrazin-2-yl]-2-methyl-but-3-yn-2-ol (I05.49, 0.68 g).

Step 3. A mixture of I05.49 (0.68 g, 2.3 mmol), toluene (7 mL), and NaOH (0.18 g, 4.6 mmol) was stirred at 120° C. for 1 h. The mixture was poured into H₂O (10 mL), extracted with EtOAc (2×15 mL), and the extracts were combined, washed with brine (10 mL), dried over Na₂SO₄, filtered, and concentrated to provide 3-(4,4-difluoropiperidin-1-yl)-5-ethynyl-2-methylpyrazine (I05.50, 0.39 g).

Alkyne Synthesis Method I05F:

### Synthesis of 3-(6-ethynyl-3-fluoropyridin-2-yl)-3-azabicyclo[3.1.0]hexane (I05.74)

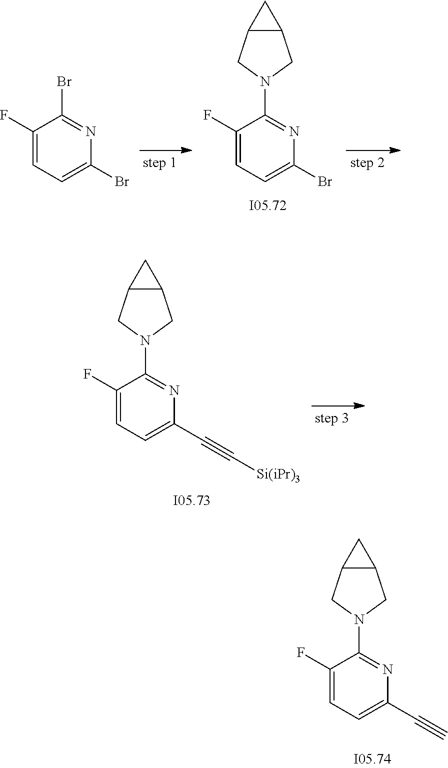

Step 1. A mixture of 2,6-dibromo-3-fluoro-pyridine (1.0 g, 4.0 mmol), 3-azabicyclo[3.1.0]hexane hydrochloride (0.48 g, 4.0 mmol), DMF (15 mL), and K₂CO₃ (1.6 g, 12 mmol) was stirred at 100° C. for 12 h, poured into H₂O (30 mL), and extracted with EtOAc (2×30 mL). The combined extracts were washed with brine (20 mL), dried over Na₂SO₄, filtered, concentrated, and purified by silica chromatography (0-100% EtOAc in PE) to provide 3-(6-bromo-3-fluoro-2-pyridyl)-3-azabicyclo[3.1.0]hexane (I05.72, 0.95 g).

Step 2. A mixture of I05.72 (0.50 g, 2.0 mmol), ethynyl(triisopropyl)silane (1.0 mL, 6.0 mmol), Pd(PPh₃)₂Cl₂ (0.14 g, 0.19 mmol), Et₃N (0.81 mL, 6.0 mmol), CuI (74 mg, 0.39 mmol), and THF (10 mL) was heated at 60° C. for 2 h in a microwave reactor. The mixture was poured into H₂O (100 mL) and extracted with EtOAc (2×100 mL). The combined extracts were washed with brine (20 mL), dried over Na₂SO₄, filtered, concentrated and purified by silica chromatography (0-100% EtOAc in PE) to provide 2-[6-(3-azabicyclo[3.1.0]hexan-3-yl)-5-fluoro-2-pyridyl]ethynyl-triisopropyl-silane (I05.73, 1.2 g).

Step 3. A mixture of I05.73 (1.1 g, 3.0 mmol), THF (12 mL), and TBAF (1 M, 9 mL, 9 mmol) was stirred at 20° C. for 2 h, then was poured into H₂O (30 mL) and extracted with EtOAc (2×30 mL). The combined extracts were washed with brine (10 mL), dried over Na₂SO₄, filtered, concentrated, and purified by silica chromatography (0-50% EtOAc in PE) to provide 3-(6-ethynyl-3-fluoropyridin-2-yl)-3-azabicyclo[3.1.0]hexane (I05.74, 0.33 g).

The alkyne in Table 4A were prepared by methods AI05-E as indicated.

TABLE 4A

| Name | Method | Structure | Protected Alkyne | Aryl Halide |
|---|---|---|---|---|
| I05.13 | I05A | 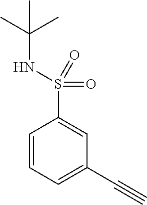 [SMILES: C#Cc1cccc(S(=O)(=O)NC(C)(C)C)c1] | 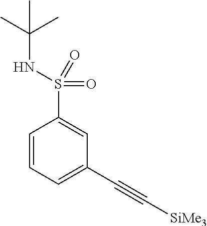 [SMILES: CC(C)(C)NS(=O)(=O)c1cccc(C#C[Si](C)(C)C)c1] | 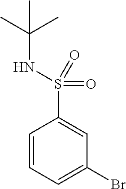 |
| I05.15 | I05A | 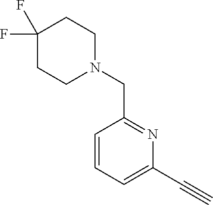 [SMILES: C#Cc1cccc(CN2CCC(F)(F)CC2)n1] | 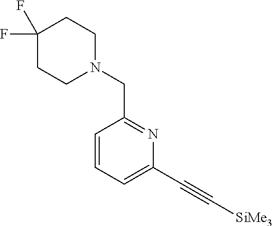 [SMILES: C[Si](C)(C)C#Cc1cccc(CN2CCC(F)(F)CC2)n1] | 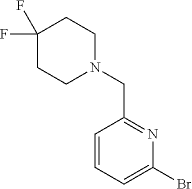 [SMILES: FC1(F)CCN(Cc2cccc(Br)n2)CC1] |
| I05.16 | I05A | 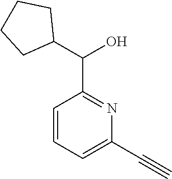 | 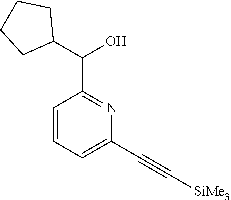 [SMILES: C[Si](C)(C)C#Cc1cccc(C(O)C2CCCC2)n1] | 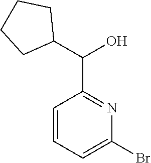 [SMILES: OC(c1cccc(Br)n1)C1CCCC1] |
| I05.21 | I05A | 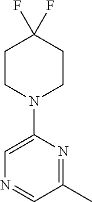 | 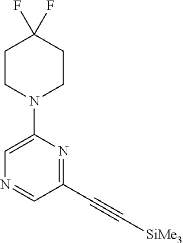 [SMILES: C[Si](C)(C)C#Cc1cncc(N2CCC(F)(F)CC2)n1] | 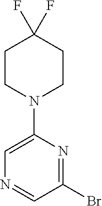 [SMILES: FC1(F)CCN(c2cncc(Br)n2)CC1] |
| I05.22 | I05C | 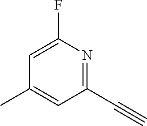 | 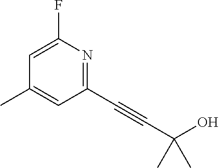 | 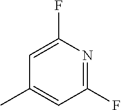 |
| I05.23 | I05D | 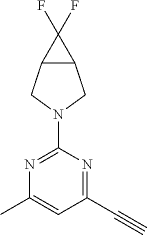 [SMILES: C#Cc1cc(C)nc(N2CC3C(C2)C3(F)F)n1] | 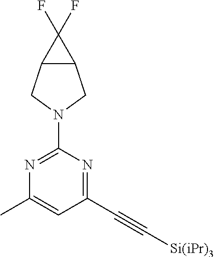 | 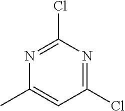 |
| I05.25 | I05C | 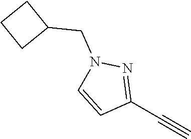 | 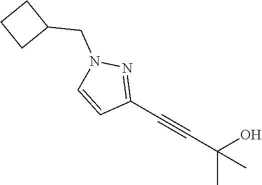 [SMILES: CC(C)(O)C#Cc1ccn(CC2CCC2)n1] | 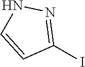 |
| I05.26 | I05D | 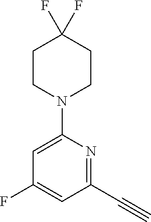 [SMILES: C#Cc1cc(F)cc(N2CCC(F)(F)CC2)n1] | 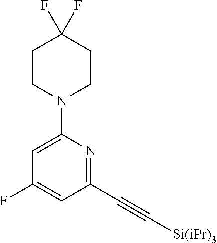 | 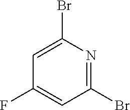 |
| I05.27 | I05C | 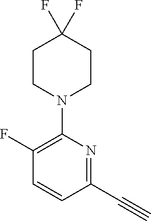 [SMILES: C#Cc1ccc(F)c(N2CCC(F)(F)CC2)n1] | 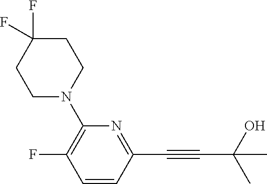 [SMILES: CC(C)(O)C#Cc1ccc(F)c(N2CCC(F)(F)CC2)n1] | 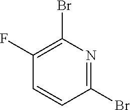 |
| I05.28 | I05A | 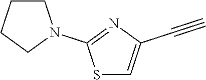 | 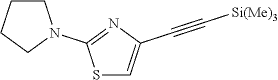 | 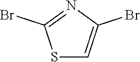 |
| I05.29 | I05C | 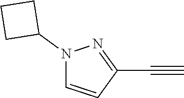 | 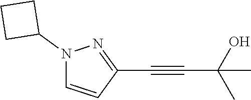 | 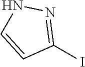 |
| I05.30 | I05C | 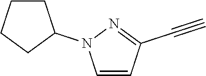 | 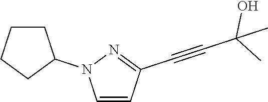 [SMILES: CC(C)(O)C#Cc1ccn(C2CCCC2)n1] | 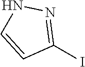 |
| I05.31 | I05D | 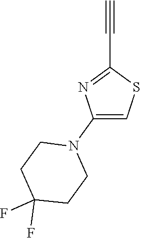 | 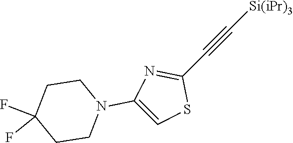 | 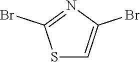 |
| I05.32 | I05D | 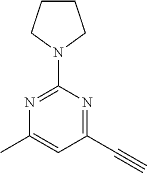 | 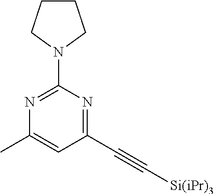 | 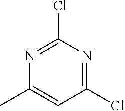 |
| I05.33 | I05D | 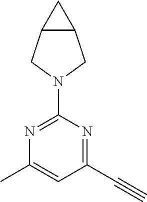 | 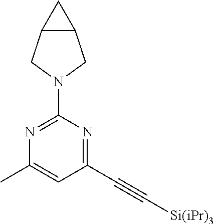 | 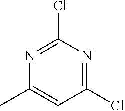 |
| I05.34 | I05D | 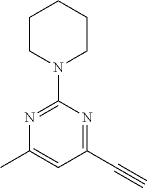 | 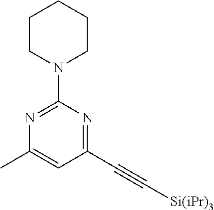 | 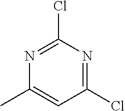 |
| I05.35 | I05D | 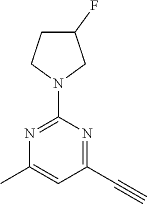 | 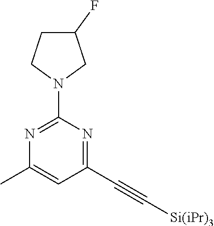 | 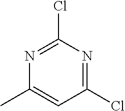 |
| I05.36 | I05D | 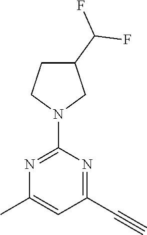 [SMILES: C#Cc1cc(C)nc(N2CCC(C(F)F)C2)n1] | 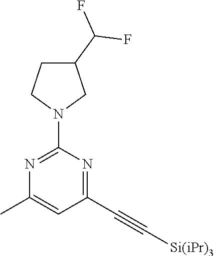 | 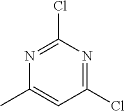 |
| I05.43 | I05B | 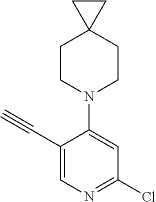 [SMILES: C#Cc1cnc(Cl)cc1N1CCC2(CC1)CC2] | 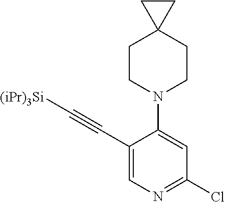 | 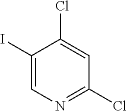 |
| I05.47 | I05B | 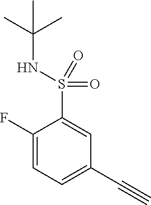 [SMILES: C#Cc1ccc(F)c(S(=O)(=O)NC(C)(C)C)c1] | 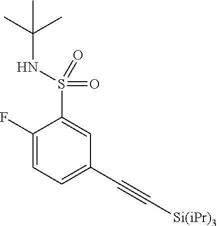 | 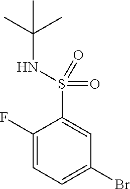 [SMILES: CC(C)(C)NS(=O)(=O)c1cc(Br)ccc1F] |
| I05.53 | I05B | 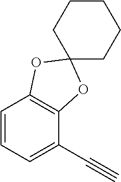 [SMILES: C#Cc1cccc2c1OC1(CCCCC1)O2] | 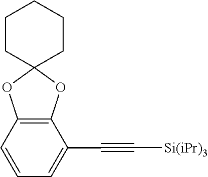 | 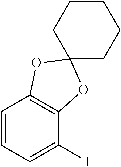 [SMILES: Ic1cccc2c1OC1(CCCCC1)O2] |
| I05.54 | I05D | 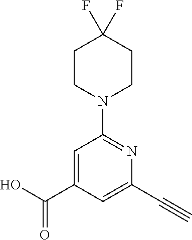 [SMILES: C#Cc1cc(C(=O)O)cc(N2CCC(F)(F)CC2)n1] | 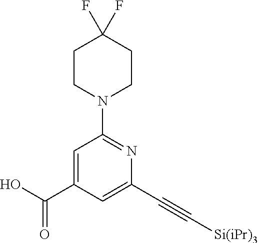 | 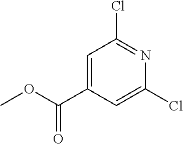 |
| I05.58 | I05B | 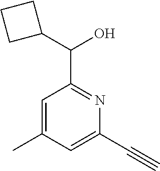 | 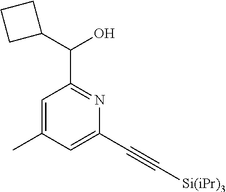 | 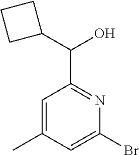 |
| I05.59 | I05B | 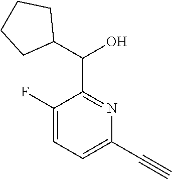 [SMILES: C#Cc1ccc(F)c(C(O)C2CCCC2)n1] | 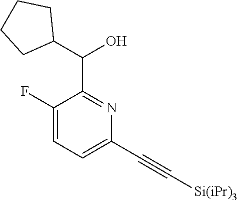 | 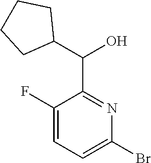 [SMILES: OC(c1nc(Br)ccc1F)C1CCCC1] |
| I05.60 | I05D | 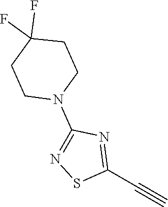 | 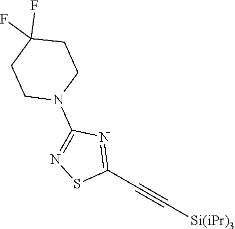 | 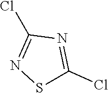 |
| I05.61 | I05C | 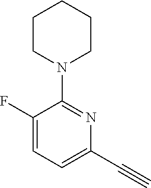 | 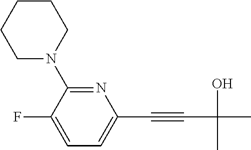 [SMILES: CC(C)(O)C#Cc1ccc(F)c(N2CCCCC2)n1] | 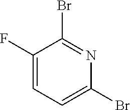 |
| I05.62 | I05B | 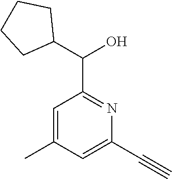 [SMILES: C#Cc1cc(C)cc(C(O)C2CCCC2)n1] | 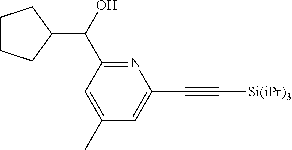 | 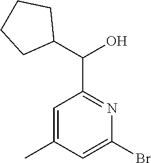 |
| I05.75 | I05F | 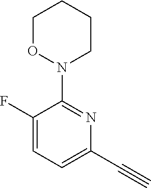 | 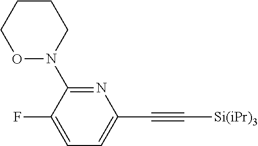 | 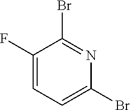 |
| I05.82 | I05D | 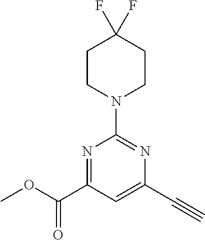 [SMILES: C#Cc1cc(C(=O)OC)nc(N2CCC(F)(F)CC2)n1] | 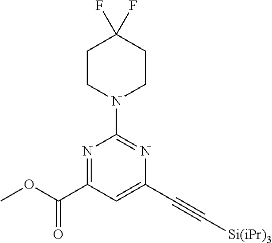 | 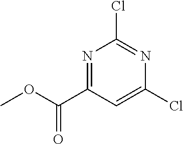 |

### Synthesis of 1-azido-4-bromo-2-fluorobenzene (I05.19)

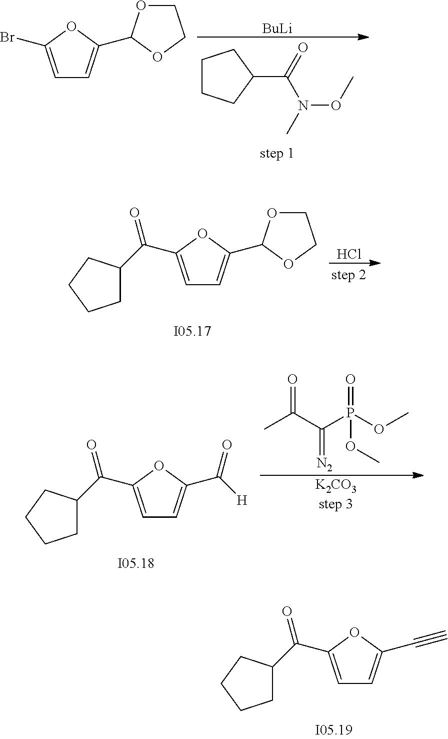

Step 1. To a mixture of 2-(5-bromo-2-furyl)-1,3-dioxolane (1.4 g, 6.4 mmol), N,N,N′,N′-tetramethylethane-1,2-diamine (0.97 mL, 6.4 mmol), and THF (20 mL) was added BuLi (1 M, 9.6 mL) at −70° C., and then N-methoxy-N-methyl-cyclopentanecarboxamide (1.5 g, 9.6 mmol) in THF (20 mL) was added dropwise at −70° C. The mixture was stirred at 20° C. for 1 h, poured into saturated NH₄Cl (10 mL), and extracted with EtOAc (2×10 mL). The combined extracts were washed with brine (10 mL), dried over Na₂SO₄, concentrated, and purified by silica chromatography (0-100% EtOAc in PE) to provide cyclopentyl-[5-(1,3-dioxolan-2-yl)-2-furyl]methanone (I05.17, 0.25 g).

Step 2. A mixture of I05.17 (0.25 g, 1.0 mmol), THF (0.8 mL), H₂O (0.5 mL), and 3M HCl (1.0 mL, 3.0 mmol) was stirred at 20° C. for 4 h. The mixture was poured into water (10 mL) and extracted with EtOAc (2×10 mL). The combined extracts were washed with brine (10 mL), dried over Na₂SO₄, concentrated, and purified by silica chromatography (0-100% EtOAc in PE) to provide 5-(cyclopentanecarbonyl)furan-2-carbaldehyde (I05.18, 0.19 g).

Step 3. A mixture of I05.18 (0.19 g, 0.99 mmol), MeOH (0.5 mL), K₂CO₃ (0.27 g, 2.0 mmol), and 1-diazo-1-dimethoxyphosphoryl-propan-2-one (0.23 g, 1.2 mmol) was stirred at 20° C. for 12 h. The mixture was poured into H₂O (10 mL) and extracted with EtOAc (2×10 mL). The combined extracts were washed with brine (10 mL), dried over Na₂SO₄, filtered, concentrated, and purified by preparative TLC (20% EtOAc in PE) to provide cyclopentyl-(5-ethynyl-2-furyl)methanone (I05.19, 0.18 g).

### Synthesis of 4-bromo-2-(6-azaspiro[2.5]octan-6-yl)benzaldehyde (I05.20)

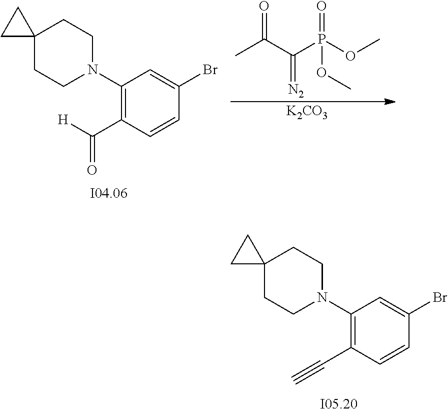

A mixture of 104.06 (0.50 g, 1.7 mmol), MeOH (10 mL), 1-diazo-1-dimethoxyphosphoryl-propan-2-one (0.39 g, 2.0 mmol), and K₂CO₃ (0.47 g, 3.4 mmol) was stirred at 20° C. for 12 h. The mixture was poured into H₂O r (30 mL) and extracted with EtOAc (2×30 mL). The combined extracts were washed with brine (10 mL), dried over Na₂SO₄, filtered, concentrated, and purified by silica chromatography (0-30% EtOAc in PE) to 4-bromo-2-(6-azaspiro[2.5]octan-6-yl)benzaldehyde (I05.20, 0.33 g).

### Synthesis of 1-(4-ethynyl-1-methyl-1H-imidazol-2-yl)-4,4-difluoropiperidine (I05.46)

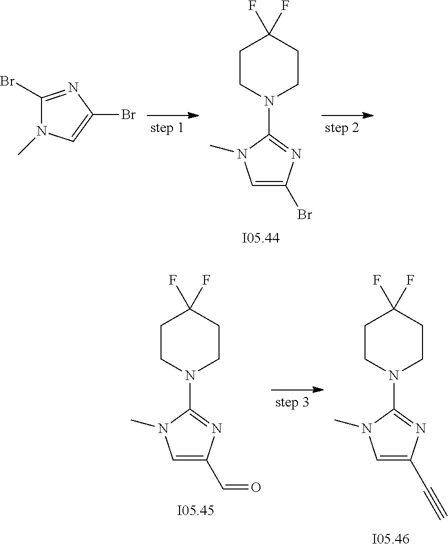

Step 1. A mixture of 2,4-dibromo-1-methyl-1H-imidazole (1.2 g, 5.0 mmol), NMP (1 mL), DBU (14 mL, 96 mmol), and 4,4-difluoropiperidine hydrochloride (4.8 g, 31 mmol) was stirred at 220° C. for 3 hours, cooled, and poured into H₂O (100 mL). The resulting mixture was extracted with EtOAc (2×100 mL), and extracts were combined, washed with brine (10 mL), dried over Na₂SO₄, filtered, concentrated, and purified by silica chromatography (0-25% EtOAc in PE) to provide 1-(4-bromo-1-methyl-1H-imidazol-2-yl)-4,4-difluoropiperidine (I05.44, 0.96 g).

Step 2. n-BuLi (2.5 M, 1.1 mL, 2.8 mmol) was added dropwise to a stirred mixture of 105.44 (0.70 g, 2.5 mmol) in THF (10 mL) under N₂ and at −78° C. After stirring at −78° C. for 20 min, DMF (0.60 mL, 7.5 mmol) was added dropwise, and the mixture was stirred at −78° C. for 15 min, warmed to 25° C., and stirred for 1 h. The reaction was quenched with H₂O and sat. NH₄Cl and extracted with EtOAc (2×50 mL). The extracts were washed with brine (20 mL), dried over Na₂SO₄, filtered, concentrated, and purified by silica chromatography (0-80% EtOAc in PE) to provide 2-(4,4-difluoropiperidin-1-yl)-1-methyl-1H-imidazole-4-carbaldehyde (I05.45, 0.18 g).

Step 3. To a mixture of I05.45 (0.17 g, 0.74 mmol) and MeOH (2 mL) was added 1-diazo-1-dimethoxyphosphoryl-propan-2-one (0.17 g, 0.89 mmol) and K₂CO₃ (0.2 g, 1.5 mmol). The mixture was stirred at 25° C. for 12 h, poured into H₂O (30 mL), extracted with EtOAc (2×30 mL), dried over Na₂SO₄, filtered, and concentrated to provide 1-(4-ethynyl-1-methyl-1H-imidazol-2-yl)-4,4-difluoropiperidine (I05.46, 0.15 g).

### Synthesis of 6-(6-bromo-3-ethynylpyridin-2-yl)-6-azaspiro[2.5]octane (I05.42)

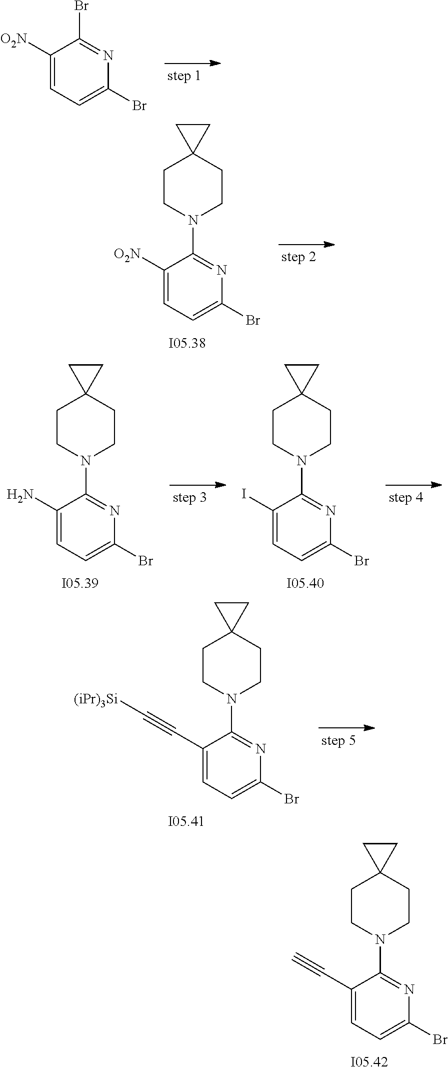

Step 1. A mixture of 2,6-dibromo-3-nitro-pyridine (1.0 g, 3.5 mmol), 6-azaspiro[2.5]octane hydrochloride (0.42 g, 3.5 mmol), EtOH (30 mL), Et₃N (1.5 mL, 11 mmol) was stirred at 25° C. for 12 h, and then was diluted with EtOAc (30 mL) and washed with H₂O (40 mL). The aqueous wash was extracted with EtOAc (20 mL). The combined extracts were washed with H₂O (20 mL) and brine (20 mL), dried over Na₂SO₄, filtered, concentrated, and purified by silica chromatography (0-8% EtOAc in PE) to provide 6-(6-bromo-3-nitropyridin-2-yl)-6-azaspiro[2.5]octane (I05.38, 0.90 g).

Step 2. To a mixture of I05.38 (0.80 g, 2.6 mmol), EtOH (24 mL), and H₂O (6 mL) was added Fe powder (1.5 g, 27 mmol) and NH₄Cl (1.4 g, 26 mmol). The mixture was stirred at 70° C. for 2 h, cooled, filtered through celite and washing the filter cake with MeOH (15 mL×3). The filtrate was concentrated and purified by silica chromatography (0-20% EtOAc in PE) to provide 6-bromo-2-(6-azaspiro[2.5]octan-6-yl)pyridin-3-amine (I05.39, 0.60 g).

Step 3. To a 0° C. mixture of I05.39 (0.430 g, 1.5 mmol) in 6 M HCl (2.6 mL, 16 mmol) was added a solution of NaNO₂ (0.14 g, 2.1 mmol) in H₂O (1 mL) over 15 min. After stirring at 0° C. for 15 min, KI (1.0 g, 6.2 mmol) in H₂O (4.5 mL) was added over 15 min. The mixture was allowed to warm to 25° C. and stirred for 1.3 h and extracted with EtOAc (25 mL×2). The combined extracts were washed with saturated NaHCO₃(15 mL×2), H₂O (15 mL), and brine (10 mL), and then was dried over Na₂SO₄, filtered, concentrated, and purified by silica chromatography (0-18% EtOAc in PE) to provide 6-(6-bromo-3-iodopyridin-2-yl)-6-azaspiro[2.5]octane (I05.40, 0.22 g).

Step 4 and Step 5 were performed as described in Alkyne Synthesis Method I05B to prepare 6-(6-bromo-3-ethynylpyridin-2-yl)-6-azaspiro[2.5]octane (I05.42) from I05.40.

### Synthesis of 2-(cyclopent-1-en-1-yl)-5-ethynylfuran (I05.52)

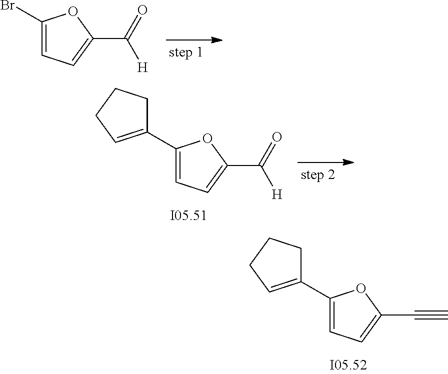

Step 1. A degassed mixture of 5-bromofuran-2-carbaldehyde (2.0 g, 11 mmol), cyclopenten-1-ylboronic acid (1.4 g, 13 mmol), K₂CO₃ (3.8 g, 27 mmol), RuPhos (0.53 g, 1.1 mmol), Pd(OAc)₂ (77 mg, 034 mmol), toluene (18 mL), H₂O (2 mL) was stirred at 120° C. for 12 h under an N₂ atmosphere. The reaction was poured into water (30 mL) and the resulting mixture was extracted with EtOAc (2×30.0 mL), and the extracts were washed with brine (10 mL), dried over Na₂SO₄, filtered, concentrated, and purified by silica chromatography (0-30% EtOAc in PE) to provide 5-(cyclopenten-1-yl)furan-2-carbaldehyde (I05.51, 1.4 g).

Step 2 was performed as described for the synthesis of I05.20 to prepare I05.52 from I05.51.

### Synthesis of N-(tert-butyl)-5-ethynylfuran-2-sulfonamide (I05.57)

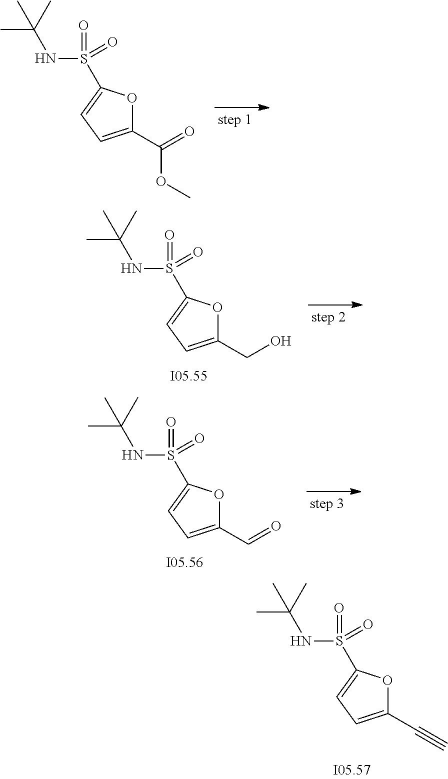

Step 1. To a 0° C. mixture of methyl 5-(tert-butylsulfamoyl)furan-2-carboxylate (0.56 g, 2.1 mmol) and THF (6 mL) was added in portions LiBH₄ (0.14 g, 6.4 mmol). The mixture was stirred at 40° C. for 1 h, cooled to 0° C., saturated NH₄Cl (8 mL) and H₂O (10 mL) were added, and the mixture was extracted with EtOAc (2×15 mL). The combined extracts were washed with brine (10 mL), dried over Na₂SO₄, and concentrated to provide N-tert-butyl-5-(hydroxymethyl)furan-2-sulfonamide (I05.55, 0.54 g).

Step 2. A mixture of I05.55 (0.30 g, 1.3 mmol), dioxane (3 mL), and MnO₂ (1.7 g, 19 mmol) was stirred at 100° C. for 1 h, cooled, filtered, combined with H₂O (15 mL), and extracted with EtOAc (15 mL×2). The combined extracts were washed with brine (10 mL), dried over Na₂SO₄, filtered and concentrated to provide N-tert-butyl-5-formyl-furan-2-sulfonamide (I05.56, 0.16 g).

Step 3 was performed as described for the synthesis of I05.20 to prepare I05.57 from 105.56.

Compounds in the following Table were prepared from the indicated aldehyde in the same manner as I05.20.

| Compound | Structure | Aldehyde |
|---|---|---|
| I05.63 | 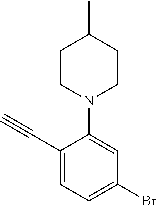 [SMILES: C#Cc1ccc(Br)cc1N1CCC(C)CC1] | 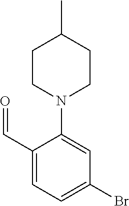 [SMILES: CC1CCN(c2cc(Br)ccc2C=O)CC1] |

### Synthesis of 2-((33-difluoroazetidin-1-yl)methyl)-4-ethynyl-6-methylpyrimidine (I05.71)

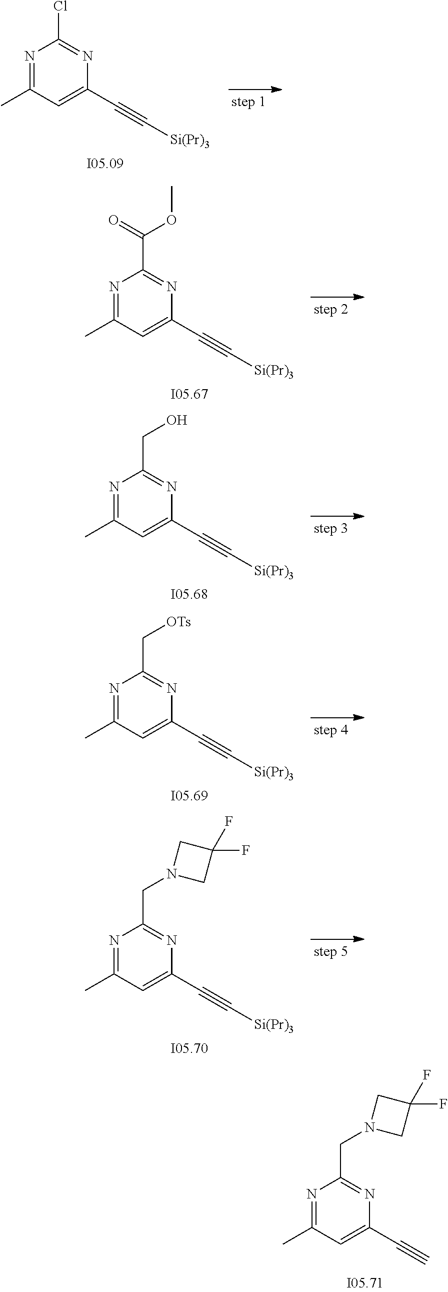

Step 1. To a mixture I05.09 (4.7 g, 15 mmol) and MeOH (10 mL) was added PdCl₂ (0.14 g, 0.76 mmol), [1-(2-diphenylphosphanyl-1-naphthyl)-2-naphthyl]-diphenyl-phosphane (947 mg, 1.5 mmol), and Et₃N (6.4 mL, 46 mmol), The mixture was stirred at 80° C. for 12 h under CO (50 psi), then the mixture was added to H₂O (10 mL) and extracted with EtOAc (2×10 mL). The extracts were combined, washed with brine (10 mL), dried over Na₂SO₄, filtered, concentrated, and purified by silica chromatography (0-30% EtOAc in PE) to methyl 4-methyl-6-((triisopropylsilyl)ethynyl)pyrimidine-2-carboxylate (I05.67, 1.9 g).

Step 2. A mixture of I05.67 (1.0 g, 3.0 mmol), EtOH (10 mL), and NaBH₄ (0.23 g, 6.0 mmol) was stirred at 0° C. for 0.5 h, then was stirred at 25° C. for 1.5 h. The mixture was poured into H₂O (30 mL) and extracted with EtOAc (2×30 mL). The extracts were combined, washed with brine (30 mL), dried over Na₂SO₄, concentrated, and purified by silica chromatography (0-30% EtOAc in PE) to provide (4-methyl-6-((triisopropylsilyl)ethynyl)pyrimidin-2-yl)methanol (I05.68, 0.90 g).

Step 3. A mixture of I05.68 (0.60 g, 2.0 mmol), CH₂Cl₂ (1.0 mL), Et₃N (0.55 mL, 4.0 mmol), 4-methylbenzenesulfonyl chloride (0.75 g, 4.0 mmol) was stirred at 0° C. for 0.5 h, then at 25° C. for 3.5 h. The mixture was combined with H₂O (10 mL) and extracted with EtOAc (10 mL×2). The extracts were combined, washed with brine (10 mL), dried over Na₂SO₄, filtered, and concentrated to provide (4-methyl-6-((triisopropylsilyl)ethynyl)pyrimidin-2-yl)methyl 4-methylbenzenesulfonate (I05.69, 1.3 g).

Step 4. A mixture of I05.69 (1.2 g, 3.0 mmol), 3,3-difluoroazetidine hydrochloride (0.36 g, 3.0 mmol), CH₃CN (10 mL), Cs₂CO₃ (1.7 g, 5.0 mmol) was stirred at 80° C. for 12 h, diluted with H₂O (30 mL), and extracted with EtOAc (30 mL×2). The extracts were washed with brine (30 mL), dried over Na₂SO₄, filtered, concentrated, and purified by silica chromatography (49-60% EtOAc in PE) to provide 2-((3,3-difluoroazetidin-1-yl)methyl)-4-methyl-6-((triisopropylsilyl)ethynyl)pyrimidine (I05.70, 0.35 g).

Step 5. A mixture of I05.70 (0.15 g, 0.40 mmol), TBAF (3.0 mL, 4.0 mmol) was stirred at 25° C. for 2 h, diluted with H₂O (10 mL), and extracted with EtOAc (10 mL×2). The combined extracts were washed with brine (10 mL), dried over Na₂SO₄, filtered, and concentrated to provide 2-((3,3-difluoroazetidin-1-yl)methyl)-4-ethynyl-6-methylpyrimidine (I05.71, 60 mg).

### Synthesis 2-(4,4-difluorocyclohex-1-en-1-yl)-4-ethynyl-6-methylpyrimidine (I05.77)

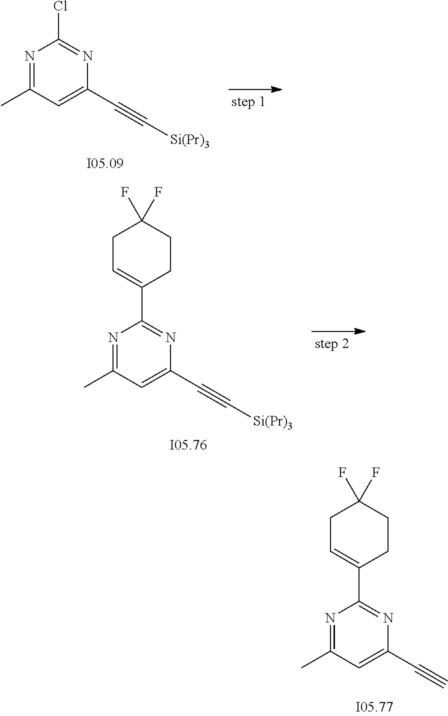

Step 1. A mixture of I05.09 (0.50 g, 2.1 mmol), dioxane (20 mL), H₂O (4.0 mL), Na₂CO₃ (0.65 g, 6.2 mmol), and Pd(dppf)Cl₂ (0.17 g, 0.21 mmol) was stirred at 100° C. for 2 h, then was poured into H₂O (30 mL), and extracted with EtOAc (2×25 mL). The extracts were combined, washed with brine (10 mL), dried over Na₂SO₄, filtered, concentrated, and purified by silica chromatography (5-50% EtOAc in PE) to provide 2-(4,4-difluorocyclohex-1-en-1-yl)-4-methyl-6-((triisopropylsilyl)ethynyl)pyrimidine (I05.76, 0.89 g).

Step 2. A mixture of I05.76 (0.20 g, 0.51 mmol), THF (2 mL), TBAF (1 M, 1.5 mL, 1.5 mmol) was stirred at 0° C. for 2 h, diluted with 30 mL of water, and extracted with EtOAc (2×30 mL). The extracts were combined, washed with brine (10 mL), dried over Na₂SO₄, filtered, and concentrated to provide 2-(4,4-difluorocyclohex-1-en-1-yl)-4-ethynyl-6-methylpyrimidine (I05.77, 0.20 g).

### Synthesis of a mixture of 2-(4,4-difluorocyclohex-1-en-1-yl)-6-ethynyl-3-fluoropyridine (I05.80) and 6-(4,4-difluorocyclohex-1-en-1-yl)-2-ethynyl-3-fluoropyridine (I05.81)

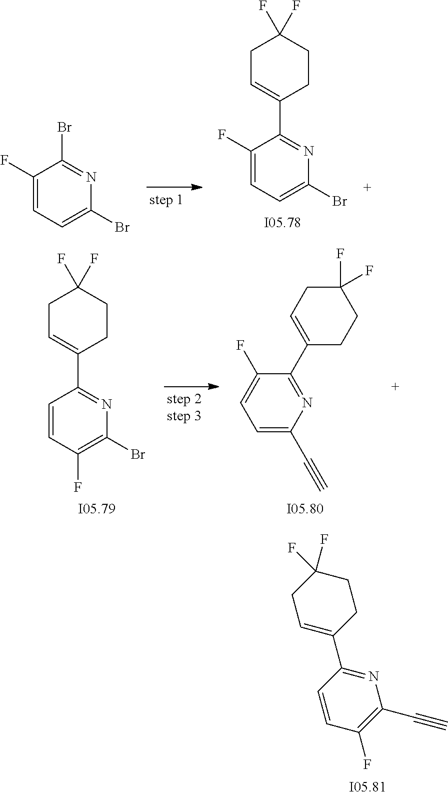

Step 1. A degassed mixture of 2,6-dibromo-3-fluoro-pyridine (1.0 g, 3.9 mmol), 2-(4,4-difluorocyclohexen-1-yl)-4,4,5,5-tetramethyl-1,3,2-dioxaborolane (0.96 g, 3.9 mmol), Na₂CO₃ (1.3 g, 12 mmol), H₂O (1 mL), dioxane (5 mL), Pd(dppf)Cl₂CH₂Cl₂ (0.32 g, 0.39 mmol) was stirred at 100° C. for 4 h, poured into H₂O (50 mL), and extracted with EtOAc (2×50 mL). The combined extracts were washed with brine (50 mL), dried over Na₂SO₄, concentrated, and purified by preparative HPLC (40-70% EtOAc in H₂O [NH₄HCO₃]) to provide 0.27 g of a mixture of 6-bromo-2-(4,4-difluorocyclohexen-1-yl)-3-fluoro-pyridine (I05.78) and 2-bromo-6-(4,4-difluorocyclohex-1-en-1-yl)-3-fluoropyridine (I05.79).

Step 2. A mixture of I05.78 & I05.79 (0.25 g, 0.86 mmol), ethynyl(triisopropyl)silane (0.58 mL, 2.6 mmol), CuI (33 mg, 0.17 mmol), Pd(PPh₃)₂Cl₂ (60 mg, 86 μmol), Et₃N (0.35 mL, 2.6 mmol), and DMF (3 mL) was heated at 60° C. for 2 h in a microwave reactor. The mixture was poured into H₂O (30 mL), extracted with EtOAc (2×30 mL), and the extracts were washed with brine (30 mL), dried over Na₂SO₄, concentrated, and purified by silica chromatography (0-30% EtOAc in PE) to provide 0.26 g of a mixture of 2-(4,4-difluorocyclohex-1-en-1-yl)-3-fluoro-6-((triisopropylsilyl)ethynyl)pyridine and 2-bromo-6-(4,4-difluorocyclohex-1-en-1-yl)-3-((triisopropylsilyl)ethynyl)pyridine.

Step 3. The product mixture from Step 2 was submitted to the conditions described in Step 2 for the synthesis of I05.77 to provide a mixture of I05.80 and 6-(4,4-difluorocyclohex-1-en-1-yl)-2-ethynyl-3-fluoropyridine (I05.81).

3-(Cyclopent-1-en-1-yl)-5-ethynyl-2-methylpyrazine (I05.83) was prepared from 3,5-dichloro-2-methylpyrazine and 2-(cyclopent-1-en-1-yl)-4,4,5,5-tetramethyl-1,3,2-dioxaborolane in the same three-step procedure described for the synthesis of I05.80 & I05.81.

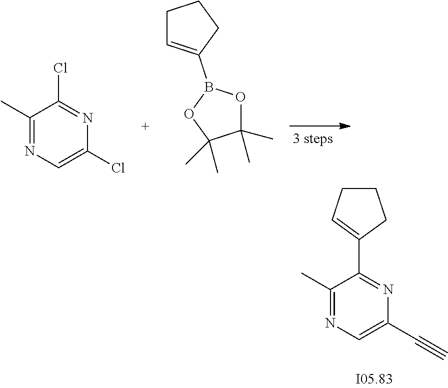

### Synthesis of 1-azido-4-bromo-2-fluorobenzene (I06.01)

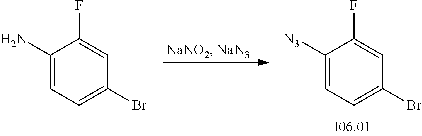 [SMILES: Nc1ccc(Br)cc1F ; [N-]=[N+]=Nc1ccc(Br)cc1F]

To a 0° C. mixture of 4-bromo-2-fluoroaniline (0.20 g, 1.1 mmol) and TFA (3 mL) was added slowly NaNO₂ (80 mg, 1.2 mmol) in portions. After stirring at 0° C. for 0.5 h, NaN₃ (80 mg, 1.2 mmol) in 1.5 mL of H₂O was added slowly. The mixture was stirred at 0° C. for 1 h, CH₂Cl₂ (15 mL) was added, and the pH was adjusted to 9 by the addition of saturated aqueous Na₂CO₃. The organic extract was washed with saturated aqueous Na₂CO₃ (2×5 mL), brine (2×5 mL), dried over Na₂SO₄, filtered, and concentrated to provide 0.22 mg of 1-azido-4-bromo-6-fluorobenzene (I06.01).

### Synthesis of 6-(3-azido-6-bromopyridin-2-yl)-6-azaspiro[2.5]octane (I06.04)

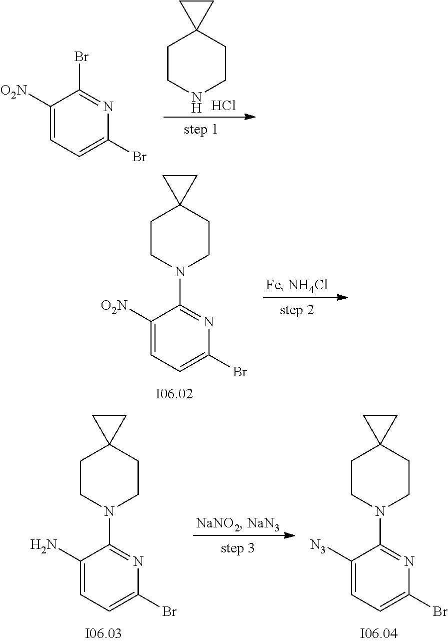

Step 1. A mixture of 2,6-dibromo-3-nitro-pyridine (2.0 g, 7.1 mmol), EtOH (20 mL), Et₃N (2.0 mL, 14 mmol), and 6-azaspiro[2.5]octane hydrochloride (1.1 g, 7.1 mmol) was stirred at 20° C. for 12 h. The mixture was poured into water (50 mL) and extracted with EtOAc (2×50 mL). The combined extracts were was washed with brine (20 mL), dried over Na₂SO₄, concentrated, and purified by silica chromatography (0-100% EtOAc in PE) to provide 6-(6-bromo-3-nitro-2-pyridyl)-6-azaspiro[2.5]octane (I06.02, 1.80 g).

Step 2. A mixture of I06.02 (0.40 g, 1.3 mmol), EtOH (3.2 mL), H₂O (0.8 mL), Fe (0.72 g, 13 mmol), and NH₄Cl (0.55 g, 10 mmol) was stirred at 80° C. for 4 h. THF (20 mL) was added, the mixture was filtered through Celite, and the filtrate was poured into water (30 mL) and extracted with EtOAc (2×30 mL). The combined extracts were washed with brine (10 mL), dried over Na₂SO₄, concentrated, purified by silica chromatography (0-100% EtOAc in Pe) to provide 2-(6-azaspiro[2.5]octan-6-yl)-6-bromo-pyridin-3-amine (I06.03, 0.29 g).

Step 3. To a 0° C. mixture of I06.03 (0.28 mg, 0.99 mmol), TFA (4.2 mL) was added NaNO₂ (75 mg, 1.1 mmol) in portions. The mixture was stirred at 0° C. for 0.75 h and a solution of NaN₃ (80 mg, 1.2 mmol) in cooled H₂O (1.4 mL) was added dropwise. The mixture was stirred at 0° C. for 2.3 hours, CH₂Cl₂ (20 mL), and the pH was adjusted to >9 by the addition of saturated Na₂CO₃ solution. The organic phase was separated, washed with saturated aqueous NaHCO₃ (20 mL×2) and brine (20 mL×2), and was dried over Na₂SO₄, filtered, concentrated, and purified by silica chromatography (10-100% EtOAc in PE) to provide 6-(3-azido-6-bromo-2-pyridyl)-6-azaspiro[2.5]octane (I06.04, 100 mg).

The aryl azides in Table 4B were prepared from the indicated 2-halo-nitroarene in the manner described for the synthesis of I06.04.

TABLE 4B

| Intermediate | Structure | amine | 2-halo-nitroarene |
|---|---|---|---|
| I06.05 | 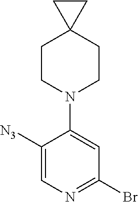 [SMILES: [N-]=[N+]=Nc1cnc(Br)cc1N1CCC2(CC1)CC2] | 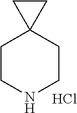 | 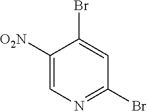 [SMILES: O=[N+]([O-])c1cnc(Br)cc1Br] |
| I06.06 | 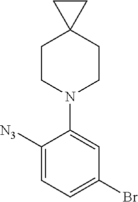 [SMILES: [N-]=[N+]=Nc1ccc(Br)cc1N1CCC2(CC1)CC2] | 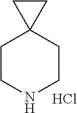 | 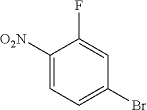 [SMILES: O=[N+]([O-])c1ccc(Br)cc1F] |
| I06.11 | 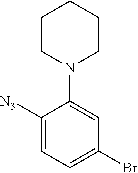 [SMILES: [N-]=[N+]=Nc1ccc(Br)cc1N1CCCCC1] | piperidine | 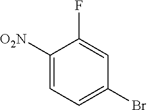 [SMILES: O=[N+]([O-])c1ccc(Br)cc1F] |
| I06.12 | 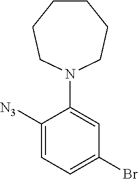 [SMILES: [N-]=[N+]=Nc1ccc(Br)cc1N1CCCCCC1] | homopiperidine | 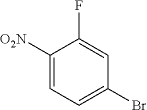 [SMILES: O=[N+]([O-])c1ccc(Br)cc1F] |
| I06.13 | 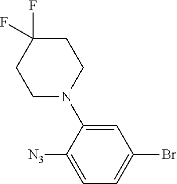 [SMILES: [N-]=[N+]=Nc1ccc(Br)cc1N1CCC(F)(F)CC1] | 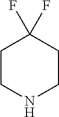 | 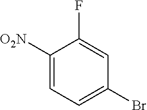 [SMILES: O=[N+]([O-])c1ccc(Br)cc1F] |
| I06.17 | 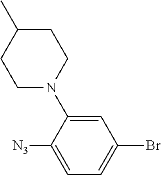 [SMILES: CC1CCN(c2cc(Br)ccc2N=[N+]=[N-])CC1] | 4-methyl piperidine | 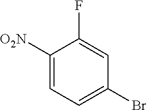 [SMILES: O=[N+]([O-])c1ccc(Br)cc1F] |
| I06.18 | 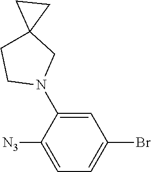 [SMILES: [N-]=[N+]=Nc1ccc(Br)cc1N1CCC2(CC2)C1] | 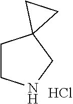 | 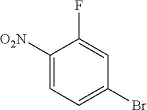 [SMILES: O=[N+]([O-])c1ccc(Br)cc1F] |
| I06.26 | 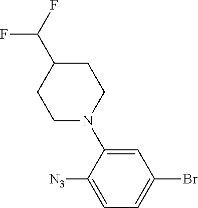 [SMILES: [N-]=[N+]=Nc1ccc(Br)cc1N1CCC(C(F)F)CC1] | 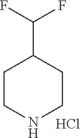 | 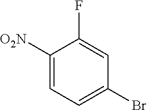 [SMILES: O=[N+]([O-])c1ccc(Br)cc1F] |
| I06.27 | 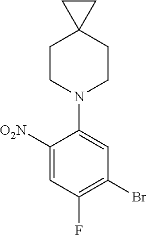 [SMILES: O=[N+]([O-])c1cc(F)c(Br)cc1N1CCC2(CC1)CC2] | 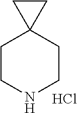 | 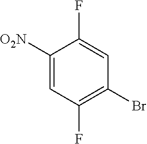 [SMILES: O=[N+]([O-])c1cc(F)c(Br)cc1F] |
| I06.30 | 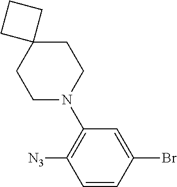 [SMILES: [N-]=[N+]=Nc1ccc(Br)cc1N1CCC2(CCC2)CC1] | 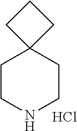 | 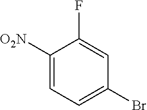 [SMILES: O=[N+]([O-])c1ccc(Br)cc1F] |

### Synthesis of 6-(5-bromo-2-nitrophenyl)-6-azaspiro[2.5]octane (I06.10)

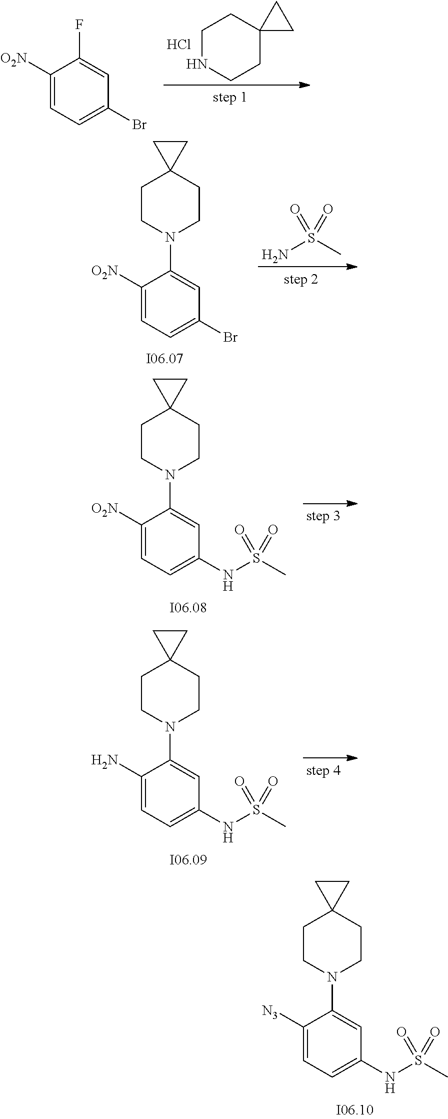

Step 1. A mixture of 6-azaspiro[2.5]octane hydrochloride (0.85 g, 5.8 mmol), 4-bromo-2-fluoro-1-nitro-benzene (1.0 g, 4.6 mmol), DMF (15 mL), and K₂CO₃ (1.9 g, 14 mmol) was stirred at 120° C. for 4 h. The mixture was combined with H₂O (40 mL) and extracted with EtOAc (20 mL×2). The combined extracts were washed with water (20 mL×3) and brine (20 mL), and dried over Na₂SO₄, filtered, concentrated, and purified by silica chromatography (0-15% EtOAc/PE) to provide the 6-(5-bromo-2-nitro-phenyl)-6-azaspiro[2.5]octane (R-029, 1.3 g, 75% purity).

Step 2. A mixture of R-029 (1.2 g, 4.0 mmol), methanesulfonamide (1.2 g, 13 mmol), CuI (0.84 g, 4.4 mmol), N¹,N²-dimethylcyclohexane-1,2-diamine (0.64 g, 4.5 mmol), K₃PO₄ (2.6 g, 12 mmol), and DMF (15 mL) was stirred under N₂ at 140° C. for 2.5 h. The mixture was combined with EtOAc (40 mL) and H₂O (30 mL) and filtered. The filtrate separated, and the organic phase was washed with H₂O (20 mL×2), brine (20 mL), dried over Na₂SO₄, was filtered, concentrated, and purified by silica chromatography (0-50% EtOAc/PE) to provide N-[3-(6-azaspiro[2.5]octan-6-yl)-4-nitro-phenyl]methanesulfonamide (R-030, 0.6 g).

Step 3. To a mixture of R-030 (0.55 g, 1.7 mmol), EtOH (25 mL), H₂O (5 mL), Fe (0.80 g, 14 mmol), and NH₄Cl (1.0 g, 19 mmol) was stirred at 90° C. for 3 h. The mixture was filtered through celite and the filtrate was concentrated, combined with EtOAc (30 mL), and washed with H₂O (15 mL×2) and brine (15 mL), and was dried over Na₂SO₄, filtered, and concentrated to provide N-[4-amino-3-(6-azaspiro[2.5]octan-6-yl)phenyl]methanesulfonamide (R-031, 0.51 g).

Step 4. To a mixture of R-031 (0.45 mg, 1.5 mmol) and MeCN (35 mL) was added a TMSN₃ (0.48 mL, 3.7 mmol) in MeCN (2.5 mL) at 0° C. After stirring at 0° C. for 0.5 h, t-butyl nitrite (0.44 mL, 3.7 mmol) in MeCN (2.5 mL) was added dropwise. The mixture was stirred at 20° C. for 9.5 h, diluted with H₂O (40 mL), extracted with CH₂Cl₂ (40 mL×2), and the combined extracts were washed with H₂O (20 mL), brine (20 mL), dried over Na₂SO₄, filtered and concentrated to provide N-[3-(6-azaspiro[2.5]octan-6-yl)-4-azido-phenyl]methanesulfonamide (I06.10, 0.48 g).

The following compounds were prepared from the indicated 2-halo-nitroarene in the same four-step procedure described for I06.10.

| Com- | | |
|---|---|---|
| pound | Structure | 2-halo-arene |
| I06.34 | 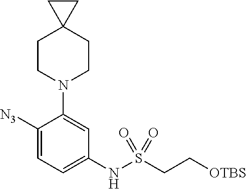 [SMILES: CC(C)(C)[Si](C)(C)OCCS(=O)(=O)Nc1ccc(N=[N+]=[N-])c(N2CCC3(CC2)CC3)c1] | 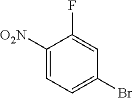 [SMILES: O=[N+]([O-])c1ccc(Br)cc1F] |

### Synthesis of 4-azido-2-(4,4-difluoropiperidin-1-yl)-6-methylpyrimidine (I06.16)

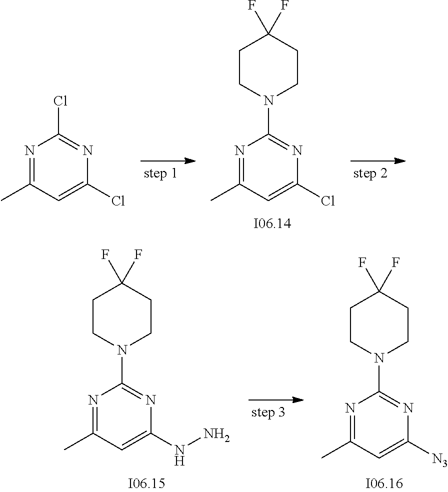

Step 1. A mixture of 2,4-dichloro-6-methyl-pyrimidine (3.0 g, 18 mmol), DMF (30 mL), Cs₂CO₃ (18 g, 55 mmol), and 4,4-difluoropiperidine (2.2 g, 18 mmol) was stirred at 100° C. for 12 h, then was poured into H₂O (50 mL) and extracted with EtOAc (2×50 mL). The combined extracts were washed with brine (50 mL), dried over Na₂SO₄, concentrated, and purified by silica chromatography (10:1-1:1 EtOAc/PE) to provide 4-chloro-2-(4,4-difluoro-1-piperidyl)-6-methyl-pyrimidine (I06.14, 0.64 g).

Step 2. A mixture of I06.14 (0.10 g, 0.40 mmol), dioxane (1.5 mL), and NH₂NH₂ monohydrate (40 μL, 0.81 mmol) was stirred at 110° C. for 4 h. The pH of was adjusted to 2 with 2M HCl and H₂O (10 mL) was added. The mixture was extracted with EtOAc (20 mL×2), and the combined extracts were washed with brine (20 mL), dried over Na₂SO₄, filtered, and concentrated to provide 2-(4,4-difluoropiperidin-1-yl)-4-hydrazineyl-6-methylpyrimidine (I06.15, 85 mg).

Step 3. To a mixture of I06.15 (85 mg, 0.35 mmol), HOAc (0.5 mL), and H₂O (0.5 mL) was added NaNO₂ (36 mg, 0.52 mmol). The mixture was stirred at 0° C. for 4 h, then was poured into ice water (20 mL) and extracted with CH₂Cl₂ (2×20 mL). The combined extracts were washed with brine (20 mL), dried over Na₂SO₄, filtered, and concentrated to provide 4-azido-2-(4,4-difluoropiperidin-1-yl)-6-methylpyrimidine (I06.16, 80 mg).

The following heterocyclic azides were prepared from the indicated heterocyclic halide and amine in the same manner as described for I06.16.

| Name | Structure | Heterocycle | Amine |
|---|---|---|---|
| I06.25 | 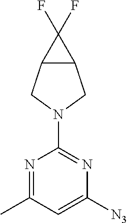 [SMILES: Cc1cc(N=[N+]=[N-])nc(N2CC3C(C2)C3(F)F)n1] | 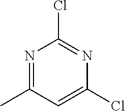 | 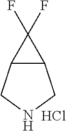 |

### Synthesis of 2-azido-6-(4,4-difluoropiperidin-1-yl)-4-methylpyridine (I06.22)

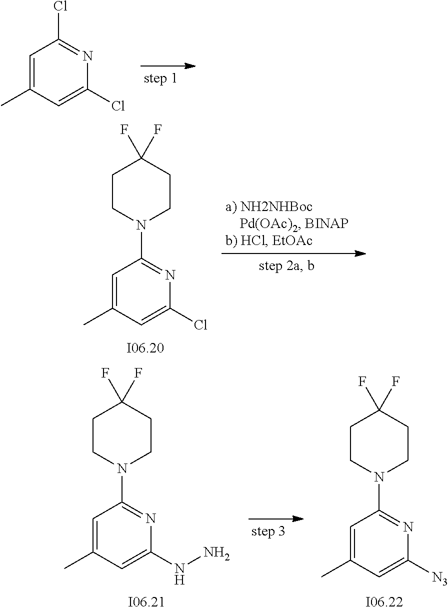

Step 1. A mixture of 2,6-dichloro-4-methyl-pyridine (1.0 g, 6.2 mmol) 4,4-difluoropiperidine hydrochloride (1.1 g, 6.8 mmol), NMP (20 mL), iPr₂NEt (4.3 mL, 25 mmol) was stirred at 140° C. for 12 h, poured into 25 mL of H₂O, and extracted with EtOAc (2×25 mL). The extracts were combined, washed with brine (10 mL), dried over Na₂SO₄, filtered, concentrated, and purified by silica chromatography (9-17% EtOAc in PE) to provide 2-chloro-6-(4,4-difluoro-1-piperidyl)-4-methyl-pyridine (I06.20, 0.95 g).

Step 2a. A degassed mixture of I06.20 (0.50 g, 2.0 mmol), BINAP (0.13 g, 0.20 mmol), Pd(OAc)₂ (45 mg, 0.20 mmol), Cs₂CO₃ (1.3 g, 4.1 mmol), BocNHNH₂ (0.40 g, 3.0 mmol) in dioxane (10 mL) was stirred at 100° C. for 12 h under an N₂ atmosphere. The mixture was poured into 15 mL of H₂O and extracted with EtOAc (2×15 mL). The extracts were combined, washed with brine (10 mL), dried over Na₂SO₄, filtered, concentrated, and purified by silica chromatography (9-17% EtOAC in PE) to provide tert-butyl N-[[6-(4,4-difluoro-1-piperidyl)-4-methyl-2-pyridyl]amino]carbamate (0.32 g).

Step 2b. To a mixture of tert-butyl N-[[6-(4,4-difluoro-1-piperidyl)-4-methyl-2-pyridyl]amino]carbamate (0.27 g, 0.79 mmol) and EtOAc (1 mL) was added HCl/EtOAc (4 M, 20 mL) and the mixture was stirred at 25° C. for 2 h. The reaction was poured into NaHCO₃(15 mL) and extracted with EtOAc (2×15 mL). The extracts were combined, washed with brine (10 mL), dried over Na₂SO₄, filtered, concentrated, and purified by silica chromatography (9-50% MeOH in CH₂Cl₂) to provide [6-(4,4-difluoro-1-piperidyl)-4-methyl-2-pyridyl]hydrazine (I06.21, 0.18 g).

Step 3. 2-azido-6-(4,4-difluoropiperidin-1-yl)-4-methylpyridine (I06.22) was prepared from I06.21 in the same manner as described for I06.16 (step 3).

The following heterocyclic azides were prepared from the indicated heterocyclic halide and amine in the same manner as described for I06.22.

| Name | Structure | Heterocycle | Amine |
|---|---|---|---|
| I06.28 | 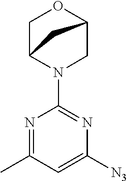 [SMILES: Cc1cc(N=[N+]=[N-])nc(N2C[C@@H]3C[C@H]2CO3)n1] | 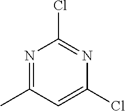 | 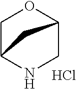 |
| I06.29 | 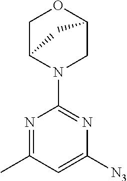 | 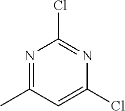 | 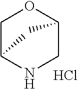 |
| I06.31 | 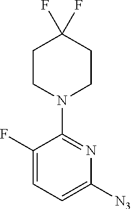 [SMILES: [N-]=[N+]=Nc1ccc(F)c(N2CCC(F)(F)CC2)n1] | 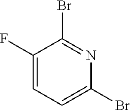 | 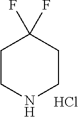 |
| I06.32 | 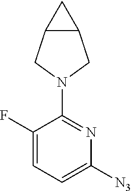 [SMILES: [N-]=[N+]=Nc1ccc(F)c(N2CC3CC3C2)n1] | 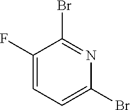 | 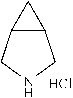 |
| I06.33 | 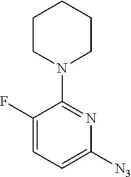 [SMILES: [N-]=[N+]=Nc1ccc(F)c(N2CCCCC2)n1] | 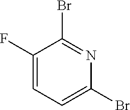 | piperidine |

### Synthesis of 2-azido-6-(4,4-difluoropiperidin-1-yl)pyrazine (I06.24)

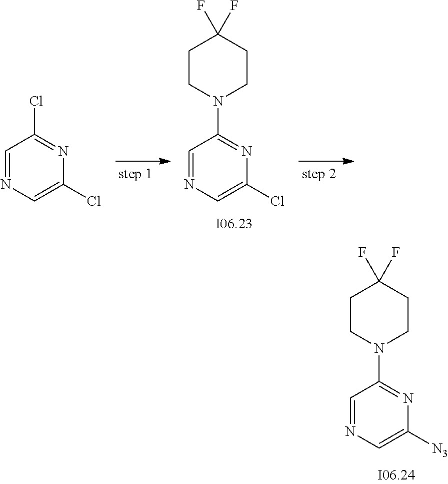 [SMILES: Clc1cncc(Cl)n1 ; FC1(F)CCN(c2cncc(Cl)n2)CC1 ; [N-]=[N+]=Nc1cncc(N2CCC(F)(F)CC2)n1]

Step 1. A mixture of 2,6-dichloropyrazine (1.0 g, 6.7 mmol), 4,4-difluoropiperidine hydrochloride (1.2 g, 7.4 mmol), K₂CO₃ (2.8 g, 20 mmol), and DMF (10 mL) was stirred at 25° C. for 2 h, then was combined with 50 mL of H₂O, and extracted with EtOAc (2×50 mL). The extracts were combined, washed with brine (20 mL), dried over Na₂SO₄, filtered, and purified by silica chromatography (0-100% EtOAc in PE) to provide 2-chloro-6-(4,4-difluoro-1-piperidyl)pyrazine (I06.23, 0.77 g).

Step 2. A degassed mixture of I06.23 (0.20 g, 0.86 mmol), DMF (5 mL), NaN₃ (0.17 g, 2.6 mmol) was stirred at 120° C. for 12 h under an N₂ atmosphere. The mixture was poured into H₂O (30 mL) and extracted with EtOAc (2×30 mL). The extracts were combined, washed with brine (10 mL), dried over Na₂SO₄, filtered, and concentrated to a volume of 2 mL. A cycle of adding 10 mL of CH₂Cl₂ and concentrating to 2 mL was repeated three times to provide a crude solution of ˜0.86 mmol of 2-azido-6-(4,4-difluoro-1-piperidyl)pyrazine (I06.24) in 2 mL of CH₂Cl₂.

### Synthesis of 1-(2-fluoro-4-nitro-phenyl)-4-(4,4,5,5-tetramethyl-1,3,2-dioxaborolan-2-yl)pyrazole (I07.02)

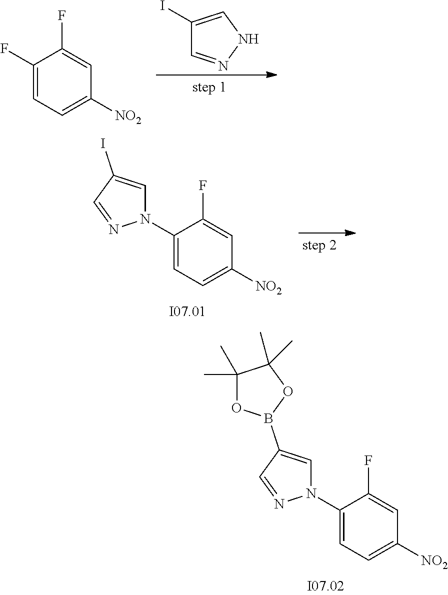

Step 1. To a mixture of 4-iodo-1H-pyrazole (2.4 g, 13 mmol) and DMSO (40 mL) was added K₂CO₃ (3.5 g, 25 mmol) and 1,2-difluoro-4-nitro-benzene (1.4 mL, 13 mmol) at 20° C. The mixture was stirred at 90° C. for 2.5 h, then was poured into H₂O (100 mL) and extracted with EtOAc (40 mL×3). The combined extracts were washed with brine (100 mL×3), dried over Na₂SO₄, filtered, and concentrated, and purified by silica chromatography (0-10% EtOH in PE) to provide 1-(2-fluoro-4-nitro-phenyl)-4-iodo-pyrazole (I07.01, 3.8 g).

Step 2. To a mixture of 107.01 (1.0 g, 3.0 mmol), bis(pinacolato)diboron (1.1 g, 4.5 mmol), and DMF (10 mL) was added KOAc (0.88 g, 9.0 mmol) and Pd(dppf)Cl₂ (0.22 g, 0.30 mmol). The mixture was stirred at 90° C. for 2 h under N₂ and was poured into H₂O (50 mL) and extracted with EtOAc mL (20 mL×3). The combined extracts were washed with brine (30 mL×3), dried over Na₂SO₄, filtered, concentrated, and purified by silica chromatography (0-15% EtOAc in PE) to provide 1-(2-fluoro-4-nitro-phenyl)-4-(4,4,5,5-tetramethyl-1,3,2-dioxaborolan-2-yl)pyrazole (I07.02, 0.64 g).

### Synthesis of 1-(3-azidophenyl)-4,4-difluoropiperidine (I09.03)

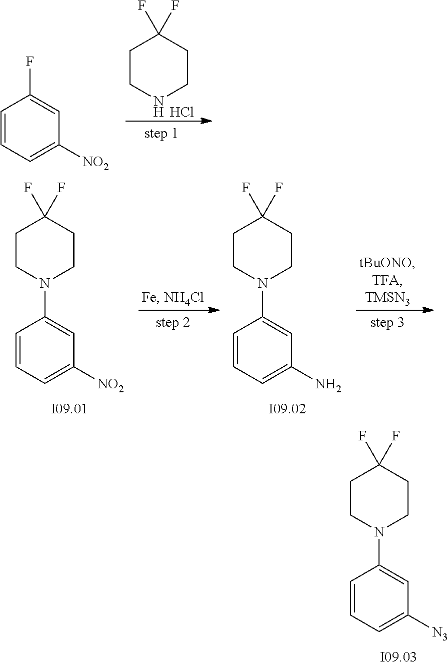

Step 1. Two mixtures of 1-fluoro-3-nitro-benzene (each 1.5 mL, 14 mmol &), DMSO (each 20 mL), K₂CO₃ (each 5.9 g, 43 mmol), and 4,4-difluoropiperidine hydrochloride (each 2.7 g, 17 mmol) were stirred at 90° C. for 12 h. The mixtures were cooled, combined, and poured into H₂O (300 mL), and extracted with EtOAc (2×300 mL). The combined extracts were washed with brine (100 mL), dried over Na₂SO₄, concentrated, and purified by silica chromatography (0-100% EtOAc in PE) to provide 4,4-difluoro-1-(3-nitrophenyl)piperidine (I09.01, 1.3 g).

Step 2. To a mixture of 109.01 (1.2 g, 5.0 mmol), EtOH (10 mL), and H₂O (2 mL) were added Fe (2.8 g, 50 mmol) and NH₄Cl (1.3 g, 25 mmol). The mixture was stirred at 80° C. for 2 h, THF (20 mL) was added, and the mixture was filtered, and the pad was washed with THF (50 mL×2). The combined filtrate was concentrated to provide 3-(4,4-difluoro-1-piperidyl)aniline (I09.02, 1.0 g).

Step 3. To a 0° C. mixture of 109.02 (1.0 g, 4.7 mmol), MeCN (10 mL) at 0° C. was added a solution of TMSN₃ (1.5 mL, 11 mmol) in MeCN (2 mL). After stirring at 0° C. for 0.5 h, a solution of tBuONO (1.3 mL, 11 mmol) in MeCN (2 mL) was added slowly. The mixture was stirred at 20° C. for 12 h, poured into ice water (300 mL), partially concentrated, and extracted with EtOAc (2×100 mL). The combined extracts were washed with brine (50 mL), dried over Na₂SO₄, and concentrated to provide 1-(3-azidophenyl)-4,4-difluoro-piperidine (I09.03, 1.0 g).

### Synthesis of 1-((5-azido-2-fluorophenyl)sulfonyl)-3,3-difluoroazetidine (109.07)

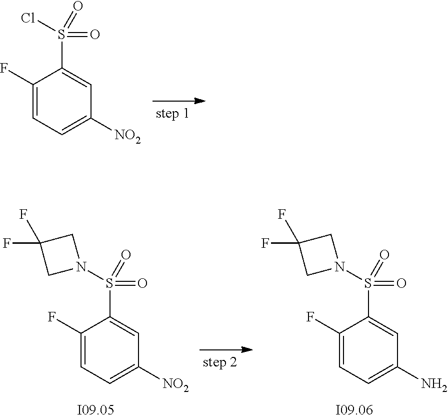 [SMILES: O=[N+]([O-])c1ccc(F)c(S(=O)(=O)Cl)c1 ; O=[N+]([O-])c1ccc(F)c(S(=O)(=O)N2CC(F)(F)C2)c1 ; Nc1ccc(F)c(S(=O)(=O)N2CC(F)(F)C2)c1]

Step 1. A 0° C. mixture of 2-fluoro-5-nitro-beznesulfonyl chloride (0.80 g, 3.3 mmol), Et₃N (0.47 mL, 3.3 mmol), and CH₂Cl₂ (20 mL) was slowly added to a stirring mixture of 3,3-difluoroazetidine hydrochloride (0.42 g, 3.2 mmol), Et₃N (1.4 mL, 10 mmol), and CH₂Cl₂ (10 mL). The new mixture was stirred for 1 h, diluted with CH₂Cl₂ (30 mL), washed with H₂O (20 mL) and brine (20 mL), dried over Na₂SO₄, filtered, concentrated, and purified by silica chromatography (0-30% EtOAc in PE) to provide 3,3-difluoro-1-((2-fluoro-5-nitrophenyl)sulfonyl)azetidine (109.05, 0.75 g).

Step 2. A mixture of 109.05 (0.69 g, 2.3 mmol), NH₄Cl (1.0 g, 19 mmol), Fe powder (1.3 g, 23 mmol), and EtOH (20 mL) was stirred at 70° C. for 10 h, cooled, and filtered through celite. The filter pad was washed with MeOH (3×10 mL) and the combined filtrate was concentrated and purified by silica chromatography (0-25% EtOAc in PE) to provide (109.06, 0.45 g).

The following compounds were prepared from the indicated anilines in the manner described for the preparation of 109.03.

| Com- | | |
|---|---|---|
| pound | Structure | Aniline |
| I09.04 | 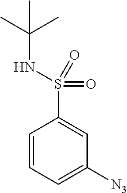 [SMILES: CC(C)(C)NS(=O)(=O)c1cccc(N=[N+]=[N-])c1] | 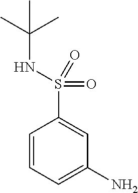 |
| I09.07 | 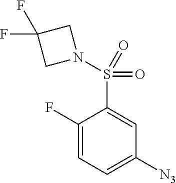 [SMILES: [N-]=[N+]=Nc1ccc(F)c(S(=O)(=O)N2CC(F)(F)C2)c1] | 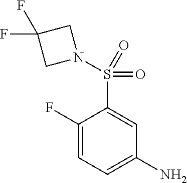 [SMILES: Nc1ccc(F)c(S(=O)(=O)N2CC(F)(F)C2)c1] |
| I09.08 | 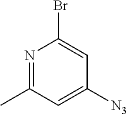 | 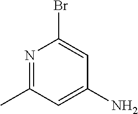 |

### Example 1: Synthesis of 4-(6-(5-(4-bromo-2-(6-azaspiro[2.5]octan-6-yl)phenyl)-4H-1,2,4-triazol-3-yl)pyridin-2-yl)morpholine (Compound 1) and N-(4-(5-(6-morpholinopyridin-2-yl)-4H-1,2,4-triazol-3-yl)-3-(6-azaspiro[2.5]octan-6-yl)phenyl)methanesulfonamide (Compound 2)

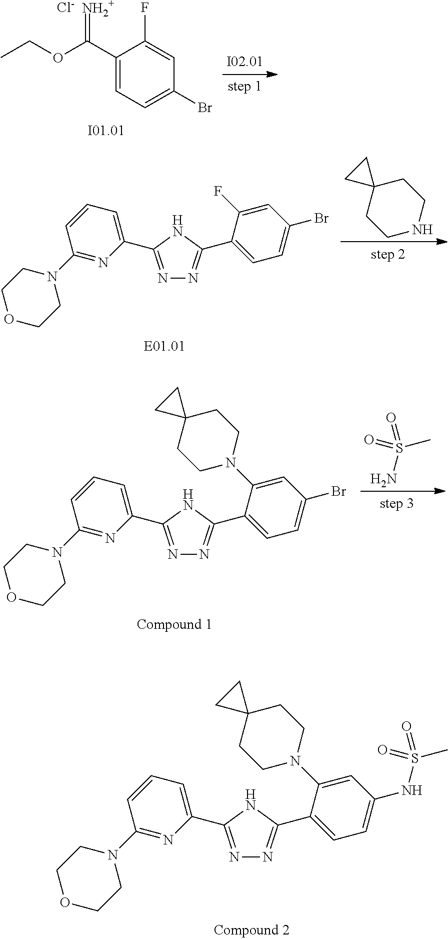

Step 1. A mixture of ethyl 4-bromo-2-fluoro-benzenecarboximidate hydrochloride (I01.01) (1.0 g, 4.0 mmol), 6-morpholinopicolinohydrazide (I02.01) (0.5 g, 2.3 mmol) and Et₃N (2.0 mL, 14 mmol) was stirred at 130° C. for 3 h, and then was concentrated and purified by silica gel chromatography (0-40% [10% MeOH in EtOAc]/PE) to provide 0.28 g of 4-[6-[5-(4-bromo-2-fluoro-phenyl)-4H-1,2,4-triazol-3-yl]-2-pyridyl]morpholine (E01.01).

Step 2. A mixture of E01.01 (0.24 g, 0.59 mmol), 6-azaspiro[2.5]octane hydrochloride (0.19 g, 1.3 mmol), K₂CO₃ (0.30 g, 2.2 mmol), and DMF (6 mL) was stirred at 140° C. in a microwave reactor for 3 h. Additional 6-azaspiro[2.5]octane hydrochloride (0.17 g, 1.2 mmol) was added and the mixture was heated at 140° C. in a microwave reactor for an additional 3 h. The mixture was combined with 30 mL of EtOAc and filtered. The filtrate was washed with H₂O (3×15 mL) and brine (15 mL), dried over Na₂SO₄, concentrated, and purified by silica chromatography (0-35% [50% THF in EtOAc]/PE) to provide 77 mg of 4-(6-(5-(4-bromo-2-(6-azaspiro[2.5]octan-6-yl)phenyl)-4H-1,2,4-triazol-3-yl)pyridin-2-yl)morpholine (Compound 1).

Step 3. A degassed mixture of Compound 1(57 mg, 0.12 mmol), methanesulfonamide (30 mg, 0.32 mmol), CuI (16 mg, 0.084 mmol), N¹,N²-dimethylcyclohexane-1,2-diamine (12 mg, 0.084 mmol), K₃PO₄ (74 mg, 0.35 mmol), and DMF (2.5 mL) was stirred at 150° C. in a microwave reactor for 2 h. The mixture was cooled, filtered, concentrated, and purified by reverse-phase HPLC (C18, 30-60%/MeCN in H₂O [1 mM NH₄CO₃]) to provide 2.5 mg of N-(4-(5-(6-morpholinopyridin-2-yl)-4H-1,2,4-triazol-3-yl)-3-(6-azaspiro[2.5]octan-6-yl)phenyl)methanesulfonamide (Compound 2).

The following compounds were prepared from the appropriate imidate ester and acyl hydrazine the manner described for the Compound 2 in Example 1.

TABLE 5

| Name | Structure | Imidate ester | Acyl hydrazine |
|---|---|---|---|
| Compound 3 | 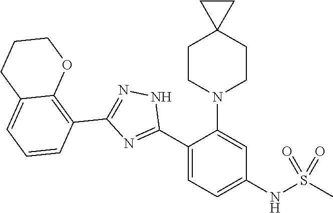 [SMILES: CS(=O)(=O)Nc1ccc(-c2nc(-c3cccc4c3OCCC4)n[nH]2)c(N2CCC3(CC2)CC3)c1] | I01.01 | I02.02 |

### Example 2: Synthesis of 4-(6-(5-(2-(6-azaspiro[2.5]octan-6-yl)pyridin-3-yl)-4H-1,2,4-triazol-3-yl)pyridin-2-yl)morpholine (Compound 4)

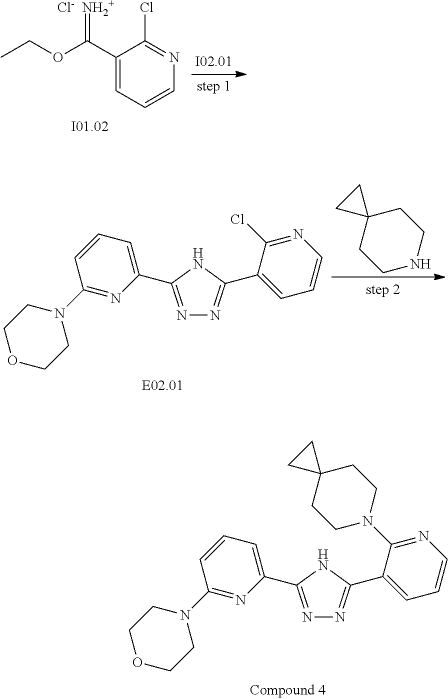

Step 1. E02.01 was prepared from I01.02 and I02.01 in the manner described for E01.01 in Example 1, step 1.

Step 2. Compound 4 was prepared from E02.01 and 6-azaspiro[2.5]octane hydrochloride in the manner described for Compound 1 in Example 1, step 2.

### Example 3. Synthesis of N-(4-(5-(6-(4,4-DIFLUOROPIPERIDIN-1-yl)pyridin-2-yl)-1H-1,2,4-TRIAZOL-3-yl)-3-(6-azaspiro[2.5]octan-6-yl)phenyl)methanesulfonamide (Compound 5)

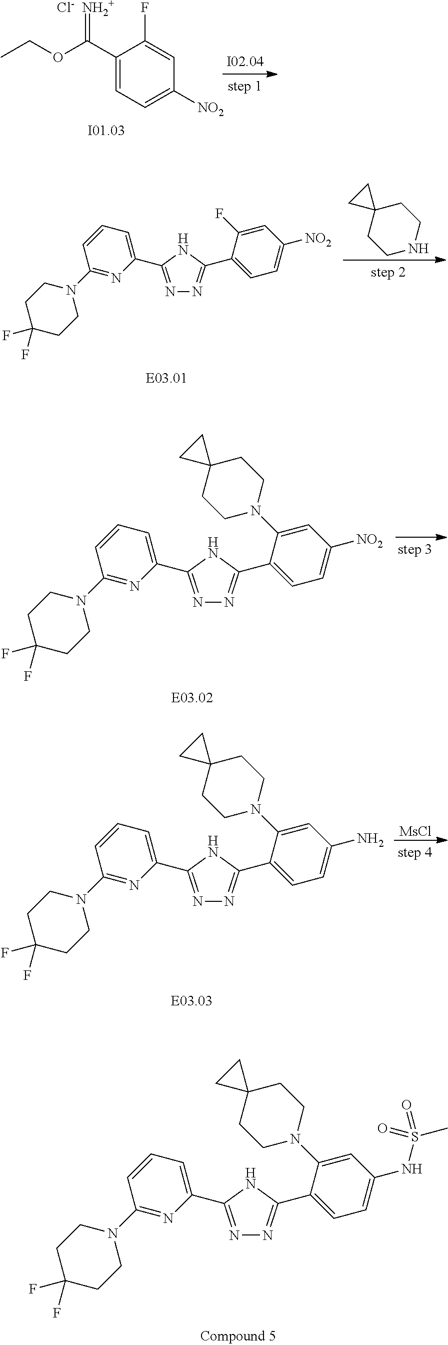

Step 1. I02.04 (0.36 g, 1.4 mmol), ethyl 2-fluoro-4-nitrobenzimidate hydrochloride (I01.03) (0.45 g, 2.1 mmol), and CH₂Cl₂ (1 mL) were combined and concentrated, and the resulting residue was combined with iPr₂NEt (1 mL) and heated to 150° C. for 1 h, then at 180° C. for 2 h, then was poured into 30 mL of H₂O and extracted with EtOAc (2×30 mL). The extracts were combined, washed with brine (10 mL), dried over Na₂SO₄, filtered, and concentrated. Purification by silica chromatography (0-100% EtOAc in PE) provided 0.86 g of 2-(4,4-difluoropiperidin-1-yl)-6-(5-(2-fluoro-4-nitrophenyl)-4H-1,2,4-triazol-3-yl)pyridine (E03.01).

Step 2. A mixture of E03.01 (0.20 g, 0.49 mmol), 6-azaspiro[2.5]octane hydrochloride (95 mg, 0.64 mmol), NMP (2 mL), and K₂CO₃ (0.21 g, 1.5 mmol) was stirred at 140° C. for 4 h and then poured into 40 mL of H₂O. The mixture was extracted with EtOAc (2×30 mL), and the combined extracts were washed with brine (10 mL), dried over Na₂SO₄, filtered, concentrated, and purified by silica chromatography (0-11% EtOAc in PE) to provide 245 mg of 6-(2-(5-(6-(4,4-difluoropiperidin-1-yl)pyridin-2-yl)-4H-1,2,4-triazol-3-yl)-5-nitrophenyl)-6-azaspiro[2.5]octane (E03.02).

Step 3. Iron powder (0.23 g, 4.0 mmol) and NH₄Cl (0.11 g, 2.0 mmol) were added to E03.02 (0.20 g, 0.40 mmol), EtOH (6 mL), and H₂O (1.2 mL) and the mixture stirred at 80° C. for 2 h. THF (30 mL) was added, and the mixture was filtered, concentrated, combined with H₂O (30 mL), and extracted with EtOAc (2×30 mL). The combined extracts were washed with brine (10 mL), dried over Na₂SO₄, filtered, concentrated, and purified by silica chromatography (0-100% EtOAc in PE) to provide 0.10 g of 4-(5-(6-(4,4-difluoropiperidin-1-yl)pyridin-2-yl)-4H-1,2,4-triazol-3-yl)-3-(6-azaspiro[2.5]octan-6-yl)aniline (E03.03).

Step 4. A mixture of E03.03 (90 mg, 0.19 mmol), CH₂Cl₂ (2 mL), MsCl (67 mg, 0.58 mmol), and pyridine (0.12 mg, 1.6 mmol) was stirred at 50° C. for 1 h, then poured into 10 mL of H₂O, and extracted with EtOAc (2×10 mL). The combined extracts were washed with brine, dried over Na₂SO₄, filtered, concentrated, and purified by reverse-phase HPLC (C18, 25-55% MeCN in H₂O [1 mM NH₄CO₃]) to provide 20 mg of N-(4-(5-(6-(4,4-difluoropiperidin-1-yl)pyridin-2-yl)-1H-1,2,4-triazol-3-yl)-3-(6-azaspiro[2.5]octan-6-yl)phenyl)methanesulfonamide (Compound 5).

### Example 4. Synthesis of N-(4-(5-(6-methyl-2-morpholinopyrimidin-4-yl)-4H-1,2,4-TRIAZOL-3-yl)-3-(6-azaspiro[2.5]octan-6-yl)phenyl)methanesulfonamide (Compound 6)

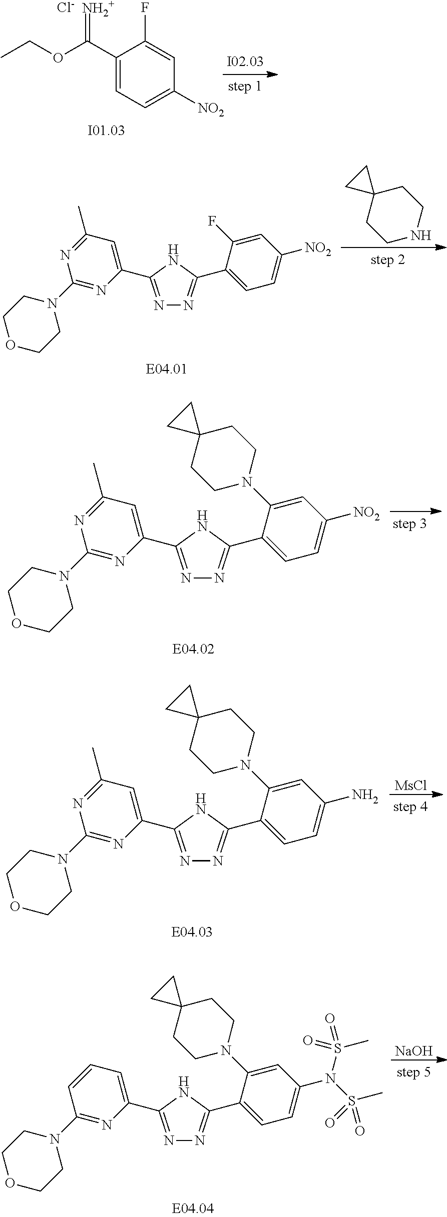

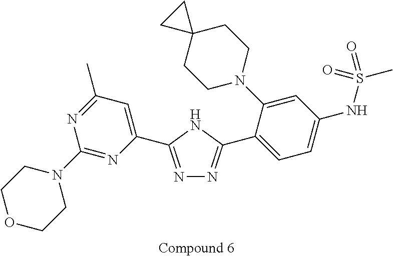 [SMILES: Cc1cc(-c2nnc(-c3ccc(NS(C)(=O)=O)cc3N3CCC4(CC3)CC4)[nH]2)nc(N2CCOCC2)n1]

Steps 1-3 were performed in the manner described for the synthesis of E03.03 (Example 3) to provide E04.03.

Step 4. A mixture of E04.03 (30 mg, 67 μmol), CH₂Cl₂ (0.2 mL), Et₃N (28 μL, 0.20 mmol), and MsCl (5 μL, 67 μmol) is stirred for 2 h, and 94 μL of additional MsCl (1.2 mmol) was added slowly at 0° C. The mixture was stirred at 20° C. for 2 h, poured in to 5 mL of saturated aqueous NaHCO₃, and extracted with 10:1 CH₂Cl₂/MeOH. (2×5 mL). The combined extract was washed with brine (5 mL), dried over Na₂SO₄, filtered, and concentrated to provide 40 mg of N-(4-(5-(6-methyl-2-morpholinopyrimidin-4-yl)-4H-1,2,4-triazol-3-yl)-3-(6-azaspiro[2.5]octan-6-yl)phenyl)-N-(methylsulfonyl)methanesulfonamide (E04.04).

Step 5. A mixture of E04.04 (27 mg, 44 μmol), THF (0.1 mL), and 2 M NaOH (44 μmol, 22 μL) was stirred for 1 h, poured into 5 mL of H₂O and the pH adjusted to 7 with 2 M HCL. The mixture was extracted with 10:1 CH₂Cl₂/MeOH (2×5 mL), and the combined extracts were washed with brine, dried over Na₂SO₄, filtered, concentrated, and purified by reverse-phase HPLC (C18, 1-40% MeCN in H₂O [0.1% formic acid]) to provide 3.7 mg of N-(4-(5-(6-methyl-2-morpholinopyrimidin-4-yl)-4H-1,2,4-triazol-3-yl)-3-(6-azaspiro[2.5]octan-6-yl)phenyl)methanesulfonamide (Compound 6).

### Example 5: Synthesis of ethyl 2-(N-(4-(5-(6-(4,4-DIFLUOROPIPERIDIN-1-yl)pyridin-2-yl)-4H-1,2,4-TRIAZOL-3-yl)-3-(6-azaspiro[2.5]octan-6-yl)phenyl)sulfamoyl)acetate (Compound 7) and N-(4-(5-(6-(4,4-DIFLUOROPIPERIDIN-1-yl)pyridin-2-yl)-4H-1,2,4-TRIAZOL-3-yl)-3-(6-azaspiro[2.5]octan-6-yl)phenyl)-2-hydroxyethane-1-sulfonamide (Compound 8)

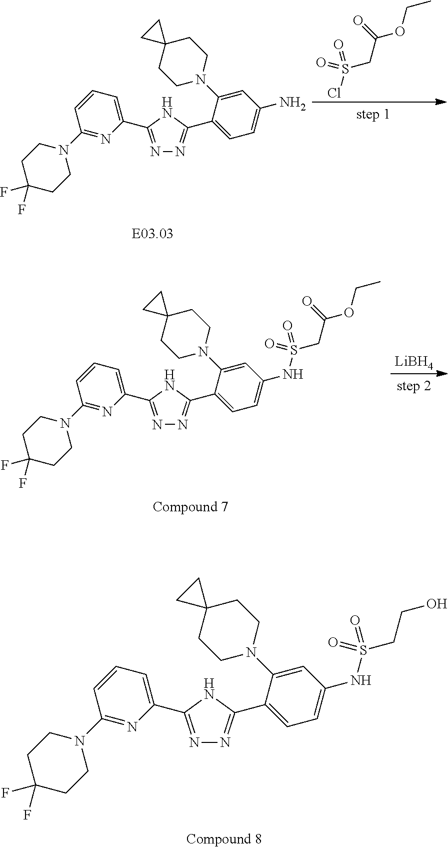

Step 1. A mixture of E03.03 (0.10 g, 0.22 mmol), ethyl 2-chlorosulfonylacetate (48 μg, 0.26 mmol), CH₂Cl₂ (2 mL), pyridine (68 mg, 0.86 mmol) was stirred for 2 h. The mixture was poured into H₂O (10 mL), extracted with EtOAc (2×10 mL). The combined extracts were washed with brine (10 mL), dried over Na₂SO₄, filtered, concentrated, and purified by silica chromatography (0-100% EtOAc in PE) to provide 80 mg of ethyl 2-(N-(4-(5-(6-(4,4-difluoropiperidin-1-yl)pyridin-2-yl)-4H-1,2,4-triazol-3-yl)-3-(6-azaspiro[2.5]octan-6-yl)phenyl)sulfamoyl)acetate (Compound 7).

Step 2. To a 0° C. mixture of Compound 7 (70 mg, 0.11 mmol) and THF (2 mL) was added LiBH₄ (7 mg, 0.34 mmol). The mixture was warmed to 20° C. and stirred for 2 h, poured into saturated aqueous NH₄Cl (20 mL), and extracted with EtOAc (2×20 mL). The combined extracts were washed with brine (10 mL), dried over Na₂SO₄, filtered, concentrated, and purified by reverse-phase HPLC (C18, 30-60% EtOAc in H₂O [0.1% formic acid]) to provide 14 mg of N-(4-(5-(6-(4,4-difluoropiperidin-1-yl)pyridin-2-yl)-4H-1,2,4-triazol-3-yl)-3-(6-azaspiro[2.5]octan-6-yl)phenyl)-2-hydroxyethane-1-sulfonamide (Compound 8).

### Example 6: Synthesis of N-(4-(5-(6-(4,4-DIFLUOROPIPERIDIN-1-yl)pyridin-2-yl)-1H-imidazol-2-yl)-3-(6-azaspiro[2.5]octan-6-yl)phenyl)methanesulfonamide (Compound 9)

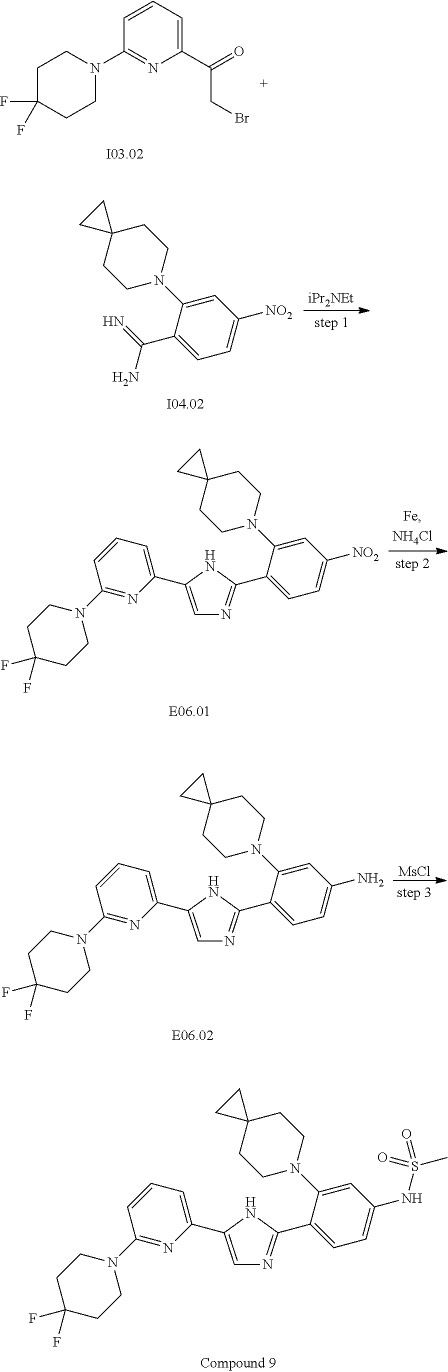

Step 1. Three separate mixtures, each of 104.02 (0.19 g, 0.69 mmol), DMF (8.5 mL), K₂CO₃ (0.22 g, 1.6 mmol), and 103.02 (0.17 g, 0.53 mmol) was stirred for 12 h. The mixtures were poured into water (20 mL), extracted with EtOAc (2×15 mL), washed with brine (20 mL), dried over Na₂SO₄, filtered, concentrated and purified by silica chromatography (0-50% EtOAc in PE) to provide 0.11 g of 6-(2-(5-(6-(4,4-difluoropiperidin-1-yl)pyridin-2-yl)-1H-imidazol-2-yl)-5-nitrophenyl)-6-azaspiro[2.5]octane (E06.01) combined.

Step 2. A mixture of E06.01 (0.11 mg, 0.22 mmol), EtOH (5 mL), H₂O (1 mL), Fe powder (0.12 g, 2.2 mmol), and NH₄Cl (59 mg, 1.1 mmol) was stirred at 80° C. for 2 h. The mixture was filtered, and the filtrate concentrated to provide 86 mg of 4-(5-(6-(4,4-difluoropiperidin-1-yl)pyridin-2-yl)-1H-imidazol-2-yl)-3-(6-azaspiro[2.5]octan-6-yl)aniline (E06.02).

Step 3. To a 0° C. mixture of E06.02 (0.10 g, 0.22 mmol) and CH₂Cl₂ (3 mL) was added MsCl (74 mg, 0.65 mmol), and Et₃N (0.13 mg, 1.3 mmol). The resulting mixture was stirred at 20° C. for 1 h, poured into H₂O (10 mL), and extracted with EtOAc (2×10 mL). The combined extracts were washed with brine (10 mL), dried over Na₂SO₄, filtered, and concentrated. The resulting residue was combined with MeOH (1.5 mL) and K₂CO₃ (0.12 g, 0.88 mmol) and stirred for 1 h, poured into H₂O (10 mL), and extracted with EtOAc (2×10 mL). The combined extracts were washed with brine (10 mL), dried over Na₂SO₄, filtered, concentrated, and purified by reverse-phase HPLC (C18, 30-53% MeCN in H₂O [0.1% formic acid]) to provide 8.5 mg of N-(4-(5-(6-(4,4-difluoropiperidin-1-yl)pyridin-2-yl)-1H-imidazol-2-yl)-3-(6-azaspiro[2.5]octan-6-yl)phenyl)methanesulfonamide (Compound 9).

Compounds in Table 6 were prepared from the appropriate amidine and α-bromoketone in the same manner as Compound 9.

TABLE 6

| Name | Structure | Amidine | α-bromoketone |
|---|---|---|---|
| Compound 10 | 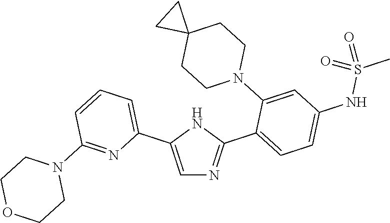 [SMILES: CS(=O)(=O)Nc1ccc(-c2ncc(-c3cccc(N4CCOCC4)n3)[nH]2)c(N2CCC3(CC2)CC3)c1] | I04.02 | I03.03 |

### Example 6A: Synthesis of N-(4-(4-(6-(cyclopentyl(hydroxy)methyl)pyridine-2-yl)-1H-1,2,3-triazol-1-yl)-3-(6-azaspiro[2.5]octan-6-yl)phenyl)methanesulfonamide (Compound 22)

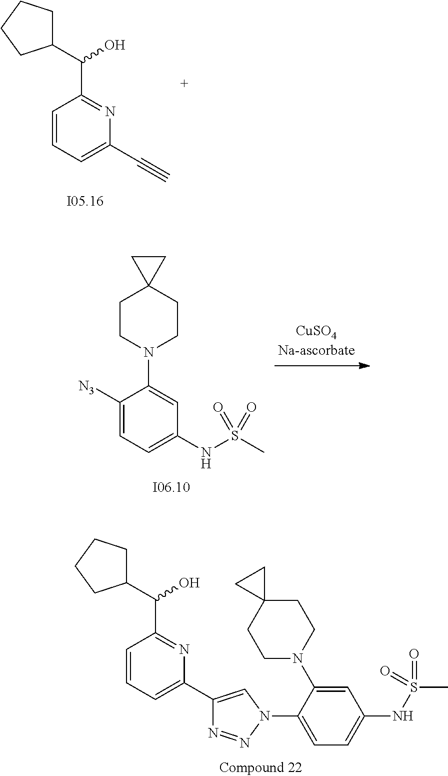 [SMILES: C#Cc1cccc(C(O)C2CCCC2)n1 ; CS(=O)(=O)Nc1ccc(N=[N+]=[N-])c(N2CCC3(CC2)CC3)c1 ; CS(=O)(=O)Nc1ccc(-n2cc(-c3cccc(C(O)C4CCCC4)n3)nn2)c(N2CCC3(CC2)CC3)c1]

A mixture of I06.10 (0.10 g, 0.31 mmol). I05.16 (70 mg, 0.35 mmol), CH₂Cl₂ (4 mL), and H₂O (4 mL), CuSO₄·5 H₂O (11 mg, 45 umol), and sodium ascorbate (80 mg, 0.40 mmol) was stirred at 25° C. for 12 h. The mixture combined with EtOAc (20 mL) and H₂O (10 mL), filtered, and the organic phase was separated and washed with H₂O (10 mL×3) and brine (10 mL), dried over Na₂SO₄, filtered, concentrated, and purified by preparative HPLC (C18; 40-75% MeCN in H₂O (NH₄HCO₃)) to provide N-(4-(4-(6-(cyclopentyl(hydroxy)methyl)pyridin-2-yl)-1H-1,2,3-triazol-1-yl)-3-(6-azaspiro[2.5]octan-6-yl)phenyl)methanesulfonamide (Compound 22, 53 mg).

Compounds in Table 6X were prepared from the appropriate alkyne and azide in the same manner as Compound 22.

TABLE 6X

| Name | Structure | Alkyne | Azide |
|---|---|---|---|
| Compound 41 | 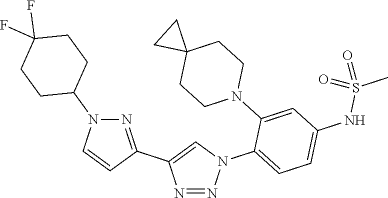 [SMILES: CS(=O)(=O)Nc1ccc(-n2cc(-c3ccn(C4CCC(F)(F)CC4)n3)nn2)c(N2CCC3(CC2)CC3)c1] | I05.24 | I06.10 |
| T6X.01 | 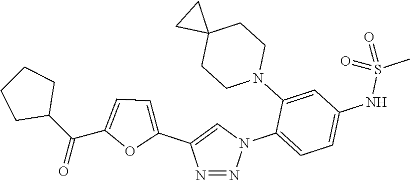 [SMILES: CS(=O)(=O)Nc1ccc(-n2cc(-c3ccc(C(=O)C4CCCC4)o3)nn2)c(N2CCC3(CC2)CC3)c1] | I05.19 | I06.10 |
| T6X.02 | 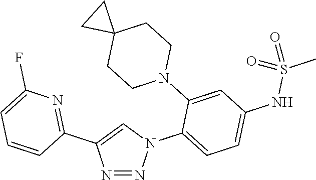 [SMILES: CS(=O)(=O)Nc1ccc(-n2cc(-c3cccc(F)n3)nn2)c(N2CCC3(CC2)CC3)c1] | 2-ethynyl-6- fluoropyridine | I06.10 |
| T6X.03 | 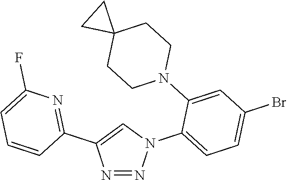 [SMILES: Fc1cccc(-c2cn(-c3ccc(Br)cc3N3CCC4(CC3)CC4)nn2)n1] | 2-ethynyl-6- fluoropyridine | I06.06 |
| T6X.04 | 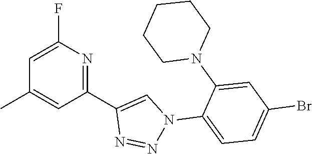 [SMILES: Cc1cc(F)nc(-c2cn(-c3ccc(Br)cc3N3CCCCC3)nn2)c1] | I05.22 | I06.11 |
| E14.01 | 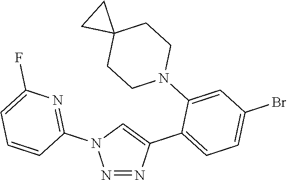 [SMILES: Fc1cccc(-n2cc(-c3ccc(Br)cc3N3CCC4(CC3)CC4)nn2)n1] | I05.20 | 2-azido-6- chloropyridine |
| T6X.06 | 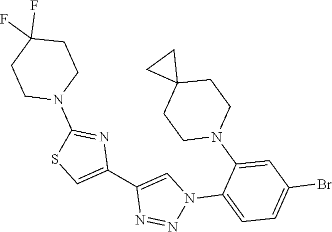 [SMILES: FC1(F)CCN(c2nc(-c3cn(-c4ccc(Br)cc4N4CCC5(CC4)CC5)nn3)cs2)CC1] | R-038 | I06.06 |
| T6X.07 | 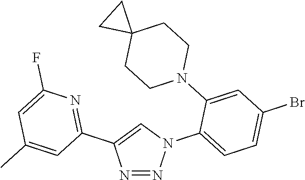 [SMILES: Cc1cc(F)nc(-c2cn(-c3ccc(Br)cc3N3CCC4(CC3)CC4)nn2)c1] | I05.22 | I06.06 |
| T6X.08 | 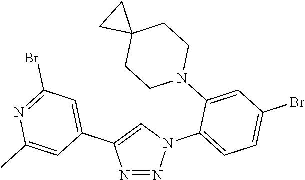 [SMILES: Cc1cc(-c2cn(-c3ccc(Br)cc3N3CCC4(CC3)CC4)nn2)cc(Br)n1] | I05.20 | I09.08 |
| Compound 63 | 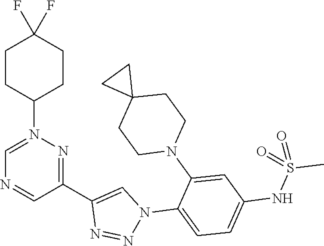 | I05.21 | I06.10 |
| T6X.09 | 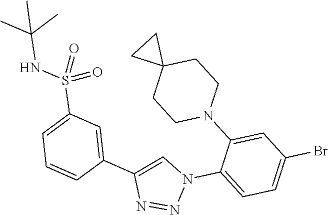 [SMILES: CC(C)(C)NS(=O)(=O)c1cccc(-c2cn(-c3ccc(Br)cc3N3CCC4(CC3)CC4)nn2)c1] | I05.13 | I06.06 |
| T6X.10 | 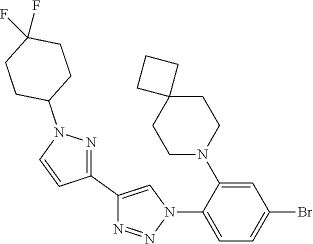 [SMILES: FC1(F)CCC(n2ccc(-c3cn(-c4ccc(Br)cc4N4CCC5(CCC5)CC4)nn3)n2)CC1] | I05.24 | I06.30 |
| T6X.11 | 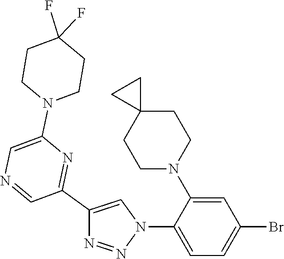 [SMILES: FC1(F)CCN(c2cncc(-c3cn(-c4ccc(Br)cc4N4CCC5(CC4)CC5)nn3)n2)CC1] | I05.21 | I06.06 |
| Compound 152 | 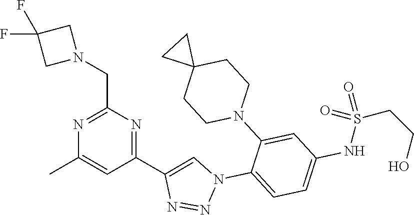 [SMILES: Cc1cc(-c2cn(-c3ccc(NS(=O)(=O)CCO)cc3N3CCC4(CC3)CC4)nn2)nc(CN2CC(F)(F)C2)n1] | I05.71 | I06.34 |
| T6X.12 | 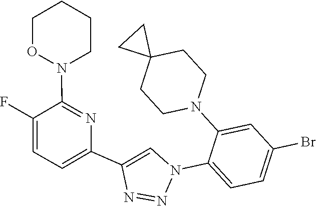 [SMILES: Fc1ccc(-c2cn(-c3ccc(Br)cc3N3CCC4(CC3)CC4)nn2)nc1N1CCCCO1] | I05.75 | I06.06 |
| T6X.13 | 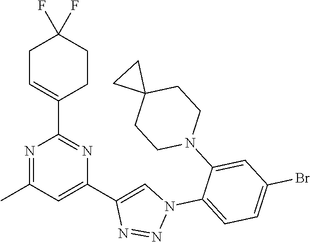 [SMILES: Cc1cc(-c2cn(-c3ccc(Br)cc3N3CCC4(CC3)CC4)nn2)nc(C2=CCC(F)(F)CC2)n1] | I05.77 | I06.06 |
| T6X.16 | 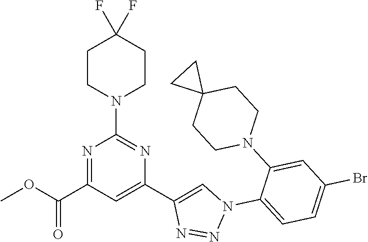 [SMILES: COC(=O)c1cc(-c2cn(-c3ccc(Br)cc3N3CCC4(CC3)CC4)nn2)nc(N2CCC(F)(F)CC2)n1] | I05.82 | I06.06 |

### Synthesis of a Mixture of 6-(5-bromo-2-(4-(6-(4,4-difluorocyclohex-1-en-1-yl)-5-fluoropyridin-2-yl)-1H-1,2,3-triazol-1-yl)phenyl)-6-azaspiro[2.5]octane (T6X.14) and 6-(5-bromo-2-(4-(6-(4,4-difluorocyclohex-1-en-1-yl)-3-fluoropyridin-2-yl)-1H-1,2,3-triazol-1-yl)phenyl)-6-azaspiro[2.5]octane (T6X.15)

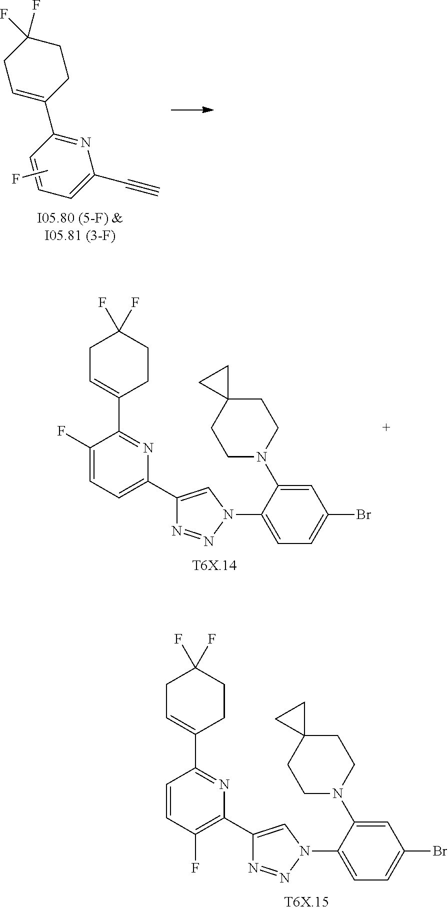

The reaction of alkynes I05.80 and I05.81 (70 mg, 0.30 mmol) and the azide I06.06 in the same manner as Compound 22 provided a mixture of 6-(5-bromo-2-(4-(6-(4,4-difluorocyclohex-1-en-1-yl)-5-fluoropyridin-2-yl)-11H-1,2,3-triazol-1-yl)phenyl)-6-azaspiro[2.5]octane (T6X.14) and 6-(5-bromo-2-(4-(6-(4,4-difluorocyclohex-1-en-1-yl)-3-fluoropyridin-2-yl)-1H-1,2,3-triazol-1-yl)phenyl)-6-azaspiro[2.5]octane (T6X.15) was. Silica chromatography (0-30% EtOAc in PE) separated the products to deliver 27 mg of T6X.14 and 70 mg of T6X.15.

### Example 7: Synthesis of N-(4-(4-(6-(4,4-DIFLUOROPIPERIDIN-1-yl)pyridin-2-yl)-1H-1,2,3-TRIAZOL-1-yl)-3-(6-azaspiro[2.5]octan-6-yl)phenyl)methanesulfonamide (Compound 11)

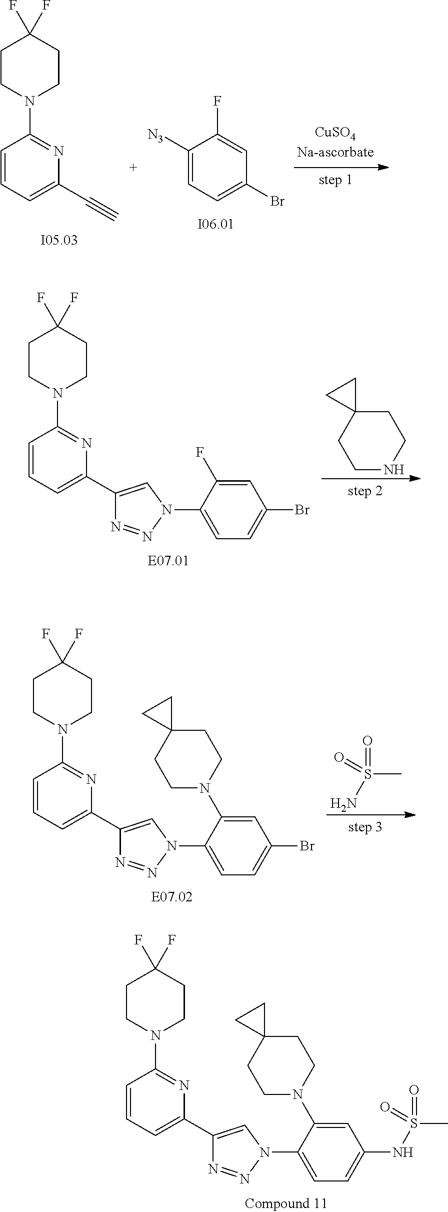

Step 1. A mixture of I06.01 (0.18 g, 0.83 mmol), I05.03 (0.37 g, 0.83 mmol), CH₂Cl₂ (2 mL), H₂O (2 mL), CuSO₄ pentahydrate (21 mg, 83 μmol), and sodium ascorbate (0.17 g, 0.83 mmol) was stirred for 12 h. The mixture was filtered, concentrated, and purified by silica chromatography (0-30% EtOAc in PE) to provide 0.10 g of 2-(1-(4-bromo-2-fluorophenyl)-1H-1,2,3-triazol-4-yl)-6-(4,4-difluoropiperidin-1-yl)pyridine (E07.01).

Step 2. A mixture of E07.01 (0.10 g, 0.23 mmol), 6-azaspiro[2.5]octane hydrochloride (67 mg, 0.46 mmol), DMF (3 mL), and K₂CO₃ (95 mg, 0.65 mmol) was stirred for 120° C. for 12 h. The mixture was filtered, concentrated, and purified by silica chromatography (0-30% EtOAc in PE) to provide 100 mg of 6-(5-bromo-2-(4-(6-(4,4-difluoropiperidin-1-yl)pyridin-2-yl)-1H-1,2,3-triazol-1-yl)phenyl)-6-azaspiro[2.5]octane (E07.02).

Step 3. A degassed mixture of E07.02 (90 mg, 0.17 mmol), methanesulfonamide (40 mg, 0.42 mmol), CuI (1.6 mg, 9 μmol), N′,N²-dimethylcyclohexane-1,2-diamine (2.4 mg, 17 μmol), K₃PO₄ (0.11 mg, 0.51 mmol), and DMF (2 mL) was stirred at 140° C. for 2 h under N₂. The mixture was combined with H₂O (10 mL) and extracted with EtOAc (3×10 mL). The extracts were combined, washed with brine (10 mL), dried over Na₂SO₄, filtered, concentrated, and purified by reverse-phase HPLC (C18, 55-80% MeCN in H₂O [HCl]) to provide N-(4-(4-(6-(4,4-difluoropiperidin-1-yl)pyridin-2-yl)-1H-1,2,3-triazol-1-yl)-3-(6-azaspiro[2.5]octan-6-yl)phenyl)methanesulfonamide (Compound 11).

Compounds in Table 6A were prepared in the same manner as Compound 11 from the appropriate alkyne and azide in step 1, amine in step 2, and sulfonamide in step 3.

TABLE 6A

| Name | Structure | Alkyne | Azide | Amine | Sulfonamide |
|---|---|---|---|---|---|
| Compound 12 | 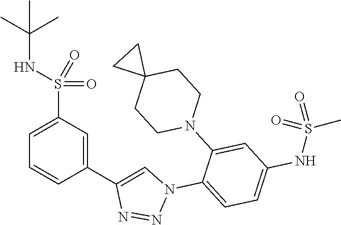 [SMILES: CC(C)(C)NS(=O)(=O)c1cccc(-c2cn(-c3ccc(NS(C)(=O)=O)cc3N3CCC4(CC3)CC4)nn2)c1] | I05.13 | I06.01 | 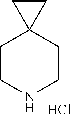 | 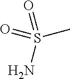 |
| Compound 13 | 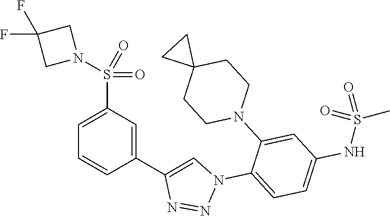 [SMILES: CS(=O)(=O)Nc1ccc(-n2cc(-c3cccc(S(=O)(=O)N4CC(F)(F)C4)c3)nn2)c(N2CCC3(CC2)CC3)c1] | I05.05 | I06.01 | 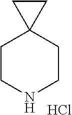 | 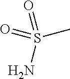 |
| Compound 14 | 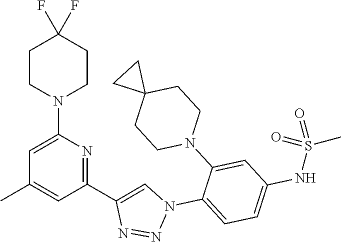 | I05.08 | I06.01 | 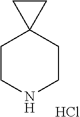 | 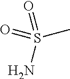 |
| Compound 15 | 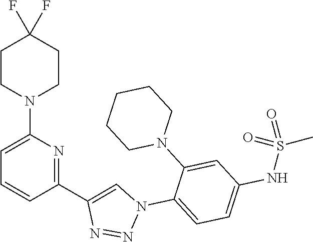 [SMILES: CS(=O)(=O)Nc1ccc(-n2cc(-c3cccc(N4CCC(F)(F)CC4)n3)nn2)c(N2CCCCC2)c1] | I05.03 | I06.01 | piperidine | 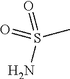 |
| Compound 18 | 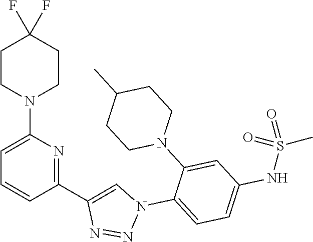 [SMILES: CC1CCN(c2cc(NS(C)(=O)=O)ccc2-n2cc(-c3cccc(N4CCC(F)(F)CC4)n3)nn2)CC1] | I05.03 | I06.01 | 4-methyl piperidine | 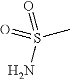 |
| Compound 19 | 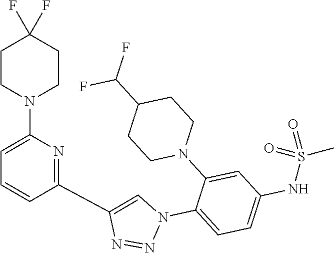 [SMILES: CS(=O)(=O)Nc1ccc(-n2cc(-c3cccc(N4CCC(F)(F)CC4)n3)nn2)c(N2CCC(C(F)F)CC2)c1] | I05.03 | I06.01 | 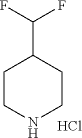 | 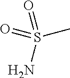 |
| Compound 20 | 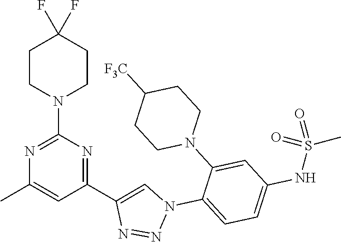 | I05.03 | I06.01 | 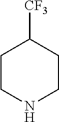 | 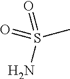 |
| Compound 21 | 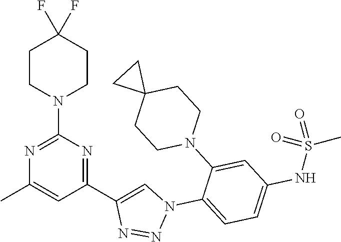 | I05.11 | I06.01 | 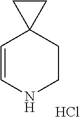 | 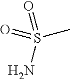 |
| Compound 23 | 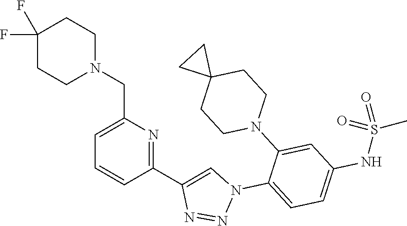 [SMILES: CS(=O)(=O)Nc1ccc(-n2cc(-c3cccc(CN4CCC(F)(F)CC4)n3)nn2)c(N2CCC3(CC2)CC3)c1] | I05.15 | I06.01 | 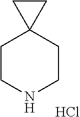 | 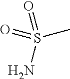 |
| Compound 47 | 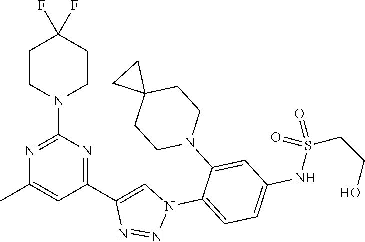 [SMILES: Cc1cc(-c2cn(-c3ccc(NS(=O)(=O)CCO)cc3N3CCC4(CC3)CC4)nn2)nc(N2CCC(F)(F)CC2)n1] | I05.11 | I06.01 | 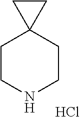 | 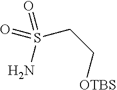 [SMILES: CC(C)(C)[Si](C)(C)OCCS(N)(=O)=O] |
| Compound 50 | 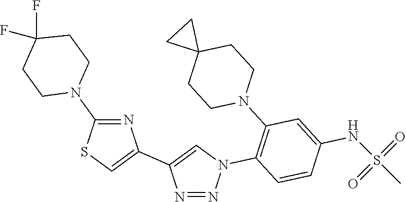 [SMILES: CS(=O)(=O)Nc1ccc(-n2cc(-c3csc(N4CCC(F)(F)CC4)n3)nn2)c(N2CCC3(CC2)CC3)c1] | R-038 | I06.01 | 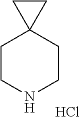 | 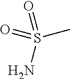 |
| Compound 92 | 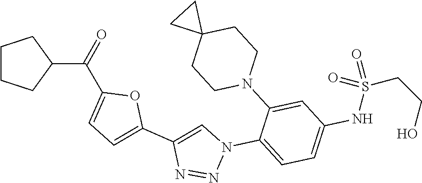 [SMILES: O=C(c1ccc(-c2cn(-c3ccc(NS(=O)(=O)CCO)cc3N3CCC4(CC3)CC4)nn2)o1)C1CCCC1] | I05.19 | I06.01 | 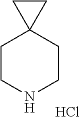 | 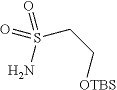 [SMILES: CC(C)(C)[Si](C)(C)OCCS(N)(=O)=O] |
| Compound 52 | 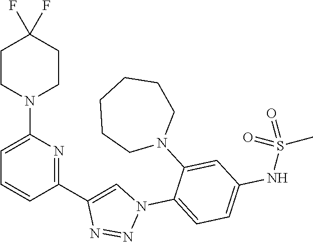 [SMILES: CS(=O)(=O)Nc1ccc(-n2cc(-c3cccc(N4CCC(F)(F)CC4)n3)nn2)c(N2CCCCCC2)c1] | I05.03 | I06.01 | 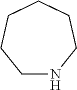 | 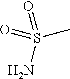 |
| Compound 53 | 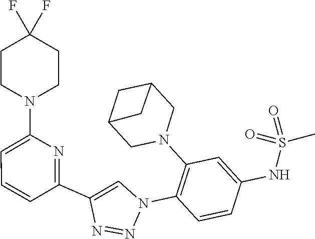 [SMILES: CS(=O)(=O)Nc1ccc(-n2cc(-c3cccc(N4CCC(F)(F)CC4)n3)nn2)c(N2CC3CC(C3)C2)c1] | I05.03 | I06.01 | 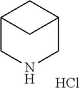 | 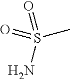 |
| T6A.03 | 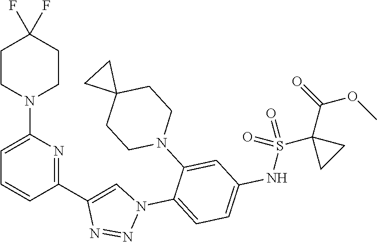 [SMILES: COC(=O)C1(S(=O)(=O)Nc2ccc(-n3cc(-c4cccc(N5CCC(F)(F)CC5)n4)nn3)c(N3CCC4(CC3)CC4)c2)CC1] | I05.03 | I06.01 | 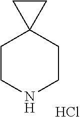 | 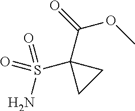 |
| T6A.04 | 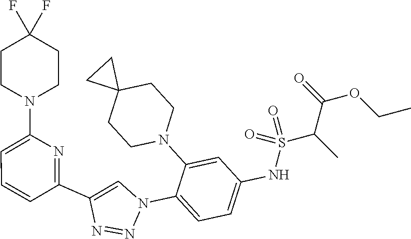 [SMILES: CCOC(=O)C(C)S(=O)(=O)Nc1ccc(-n2cc(-c3cccc(N4CCC(F)(F)CC4)n3)nn2)c(N2CCC3(CC2)CC3)c1] | I05.03 | I06.01 | 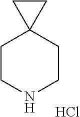 | 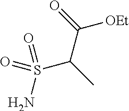 |
| Compound 69 | 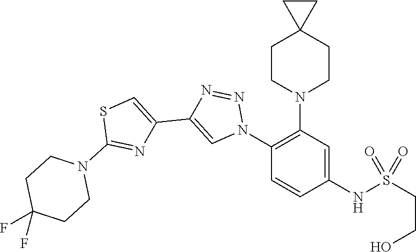 [SMILES: O=S(=O)(CCO)Nc1ccc(-n2cc(-c3csc(N4CCC(F)(F)CC4)n3)nn2)c(N2CCC3(CC2)CC3)c1] | R-038 | I06.01 | 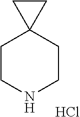 | 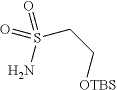 [SMILES: CC(C)(C)[Si](C)(C)OCCS(N)(=O)=O] |
| Compound 106 | 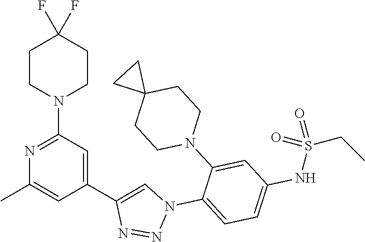 [SMILES: CCS(=O)(=O)Nc1ccc(-n2cc(-c3cc(C)nc(N4CCC(F)(F)CC4)c3)nn2)c(N2CCC3(CC2)CC3)c1] | I05.11 | I06.01 | 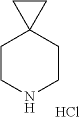 | 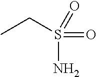 |
| Compound 116 | 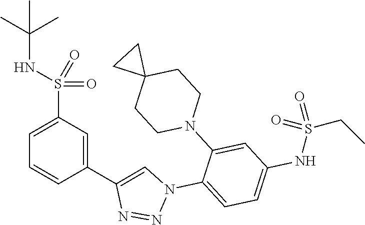 [SMILES: CCS(=O)(=O)Nc1ccc(-n2cc(-c3cccc(S(=O)(=O)NC(C)(C)C)c3)nn2)c(N2CCC3(CC2)CC3)c1] | I05.13 | I06.01 | 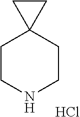 | 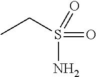 |
| Compound 151 | 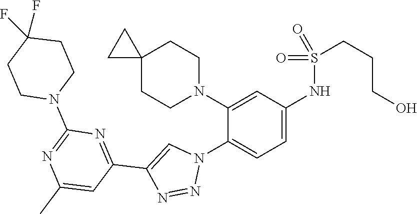 [SMILES: Cc1cc(-c2cn(-c3ccc(NS(=O)(=O)CCCO)cc3N3CCC4(CC3)CC4)nn2)nc(N2CCC(F)(F)CC2)n1] | I05.11 | I06.01 | 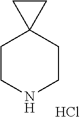 | 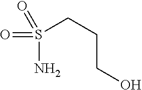 |

### Example 7A: Synthesis of N-(4-(4-(6-(4,4-difluoropiperidin-1-yl)pyridin-2-yl)-1H-1,2,3-triazol-1-yl)-3-(6-azaspiro[2.5]octan-6-yl)phenyl)-2-hydroxyethane-1-sulfonamide (Compound 45)

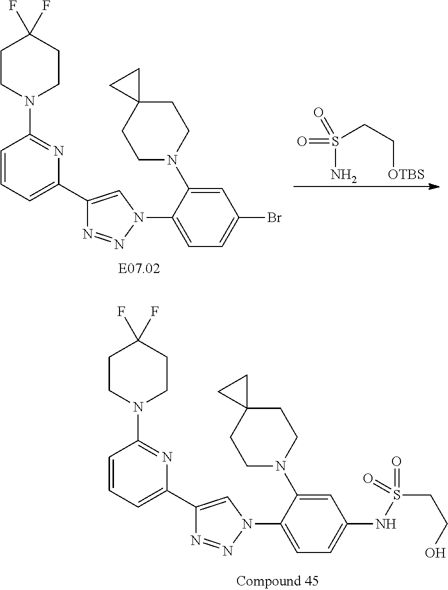 [SMILES: FC1(F)CCN(c2cccc(-c3cn(-c4ccc(Br)cc4N4CCC5(CC4)CC5)nn3)n2)CC1 ; CC(C)(C)[Si](C)(C)OCCS(N)(=O)=O ; O=S(=O)(CCO)Nc1ccc(-n2cc(-c3cccc(N4CCC(F)(F)CC4)n3)nn2)c(N2CCC3(CC2)CC3)c1]

A degassed mixture of E07.02 (0.20 g, 0.38 mmol), 2-[tert-butyl(dimethyl)silyl]oxyethane sulfonamide (0.27 g, 1.1 mmol), CuI (80 mg, 0.42 mmol), N¹,N²-dimethylcyclohexane-1,2-diamine (60 mg, 0.42 mmol), K₃PO₄ (0.24 g, 1.1 mmol), and DMF (4 mL) was stirred under N₂ at 140° C. for 4 h. A separate mixture was prepared in the same manner from 20 mg of E07.02. The two mixtures were combined, filtered, and the filtrate was diluted with EtOAc (40 mL). The mixture was washed with water (20 mL×3) and brine (20 mL), dried over Na₂SO₄, filtered, concentrated, and purified by preparative HPLC (C18: 40-70% MeCN in H₂O [10 mM NH₄HCO₃]) to provide N-(4-(4-(6-(4,4-difluoropiperidin-1-yl)pyridin-2-yl)-1H-1,2,3-triazol-1-yl)-3-(6-azaspiro[2.5]octan-6-yl)phenyl)-2-hydroxyethane-1-sulfonamide (Compound 45, 50 mg).

### Example 7B: Synthesis of N-(4-(4-(2-(4,4-difluoropiperidin-1-yl)-5-fluorothiazol-4-yl)-1H-1,2,3-triazol-1-yl)-3-(6-azaspiro[2.5]octan-6-yl)phenyl)-2-hydroxyethane-1-sulfonamide (Compound 93)

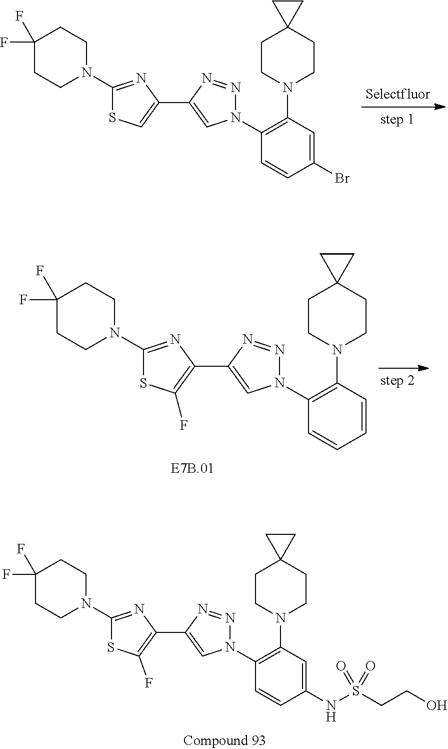 [SMILES: FC1(F)CCN(c2nc(-c3cn(-c4ccc(Br)cc4N4CCC5(CC4)CC5)nn3)cs2)CC1 ; Fc1sc(N2CCC(F)(F)CC2)nc1-c1cn(-c2ccccc2N2CCC3(CC2)CC3)nn1 ; O=S(=O)(CCO)Nc1ccc(-n2cc(-c3nc(N4CCC(F)(F)CC4)sc3F)nn2)c(N2CCC3(CC2)CC3)c1]

Step 1. To a mixture of T6X.06 (0.90 g, 2 mmol) and DMF (15 mL) was added 1-(chloromethyl)-4-fluoro-1,4-diazabicyclo[2.2.2]octane-1,4-diium ditetrafluoroborate (0.60 g, 2 mmol) and 2,6-dimethylpyridine (0.39 mL, 3 mmol) at 0° C. The mixture was stirred at 25° C. for 12 h and poured into H₂O (60 mL) and the extracted with EtOAc (2×35 mL). The combined extracts were washed with brine (10 mL), dried over Na₂SO₄, filtered, concentrated, and purified by silica chromatography (5-50% EtOAc in PE) to provide 4-[1-[2-(6-azaspiro[2.5]octan-6-yl)-4-bromo-phenyl]triazol-4-yl]-2-(4,4-difluoro-1-piperidyl)-5-fluoro-thiazole (E7B.01, 0.56 g).

Step 2. A mixture of E7B.01 (0.46 g, 0.83 mmol) and 2-hydroxyethanesulfonamide (0.21 g, 2 mmol), DMF (10 mL), CuI (0.11 g, 0.58 mmol), N¹,N₂-dimethylcyclohexane-1,2-diamine (83 mg, 0.58 mmol), and K₃PO₄ (0.53 g, 2 mmol) was stirred at 130° C. for 2 h, poured into 40 mL of H₂O, and extracted with EtOAc (2×15 mL). The combined extracts were washed with brine (20 mL), dried over Na₂SO₄, filtered, concentrated, and purified by preparative HPLC (C18; 45-65% MeCN in H₂O [NH₄HCO₃]) to provide N-(4-(4-(2-(4,4-difluoropiperidin-1-yl)-5-fluorothiazol-4-yl)-1H-1,2,3-triazol-1-yl)-3-(6-azaspiro[2.5]octan-6-yl)phenyl)-2-hydroxyethane-1-sulfonamide (Compound 93, 0.18 g).

### Example 8: Synthesis of N-(tert-butyl)-3-(2-(4-(methylsulfonamido)-2-(6-azaspiro[2.5]octan-6-yl)phenyl)-1H-imidazol-5-yl)benzenesulfonamide (Compound 34)

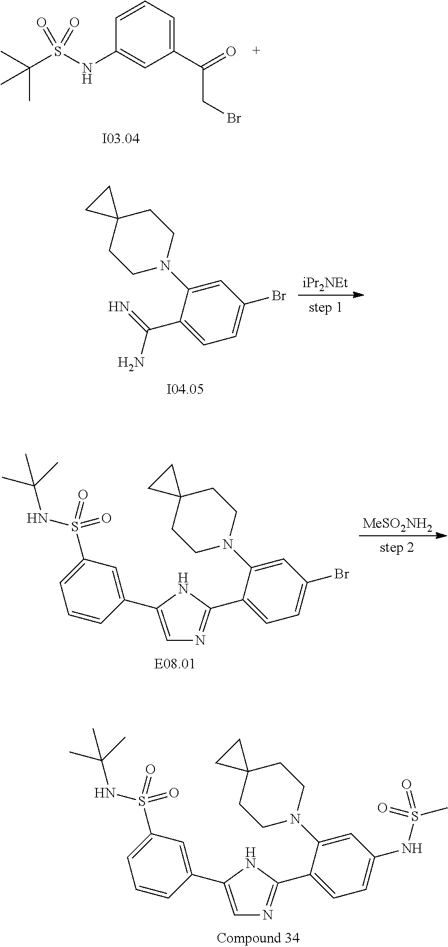

Step 1. A mixture of 103.04 (0.20 g, 0.60 mmol), THF (8 mL), and iPr₂NEt (0.45 mL, 2.6 mmol) was stirred at 80° C. for 15 min and 104.05 (0.19 g, 0.60 mmol) was added. The mixture was stirred at 80° C. for 12 h. A separate mixture was prepared from 19 mg of 103.04 and 19 mg of 104.05. The two reaction mixtures were combined and diluted with EtOAc (20 mL), washed with brine (10 mL×2), dried over Na₂SO₄, filtered, concentrated, and purified by prep-TLC (33% EtOAc in PE) to provide 3-[2-[2-(6-azaspiro[2.5]octan-6-yl)-4-bromo-phenyl]-1H-imidazol-5-yl]-N-tert-butyl-benzenesulfonamide (E07.01, 85 mg, 65% purity).

Step 2. A mixture of E07.01 (65 mg, 0.12 mmol), methanesulfonamide (58 mg, 0.61 mmol), CuI (26 mg, 0.14 mmol), N¹,N²-dimethylcyclohexane-1,2-diamine (20 mg, 0.14 mmol), K₃PO₄ (78 mg, 0.37 mmol), and DMF (2 mL) was stirred under N₂ at 120° C. for 3.5 h. A separate mixture was prepared in the same manner from 10 mg of E07.01. Both mixtures were combined and filtered. The filtrate was concentrated and purified by preparative HPLC (C18, 40-70% MeCN in H₂O [10 mM NH₄HCO₃]) to provide N-(tert-butyl)-3-(2-(4-(methylsulfonamido)-2-(6-azaspiro[2.5]octan-6-yl)phenyl)1H-imidazol-5-yl)benzenesulfonamide (Compound 34, 20 mg).

Compounds in Table 6B were prepared from the appropriate amidine, α-bromoketone, and sulfonamide in the same manner as Compound 34.

TABLE 6B

| | | | α-bromo- | |
|---|---|---|---|---|
| Name | Structure | Amidine | ketone | sulfonamide |
| Compound 26 | 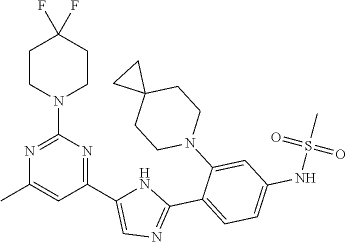 [SMILES: Cc1cc(-c2cnc(-c3ccc(NS(C)(=O)=O)cc3N3CCC4(CC3)CC4)[nH]2)nc(N2CCC(F)(F)CC2)n1] | I04.05 | I03.16 | 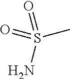 |
| Compound 28 | 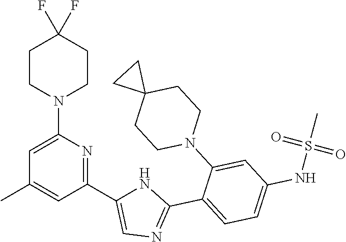 [SMILES: Cc1cc(-c2cnc(-c3ccc(NS(C)(=O)=O)cc3N3CCC4(CC3)CC4)[nH]2)nc(N2CCC(F)(F)CC2)c1] | I04.05 | I03.13 | 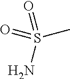 |
| Compound 30 | 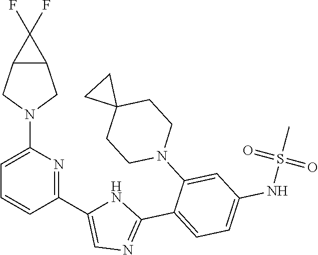 [SMILES: CS(=O)(=O)Nc1ccc(-c2ncc(-c3cccc(N4CC5C(C4)C5(F)F)n3)[nH]2)c(N2CCC3(CC2)CC3)c1] | I04.05 | I03.10 | 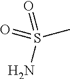 |
| Compound 32 | 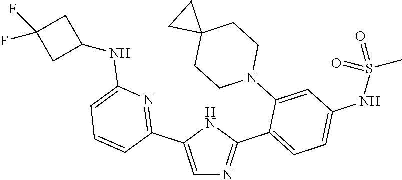 [SMILES: CS(=O)(=O)Nc1ccc(-c2ncc(-c3cccc(NC4CC(F)(F)C4)n3)[nH]2)c(N2CCC3(CC2)CC3)c1] | I04.05 | I03.09 | 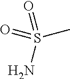 |
| E08.02 | 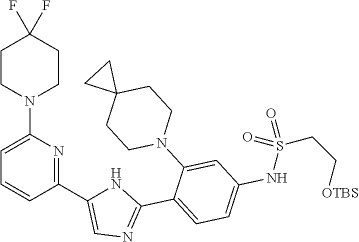 [SMILES: CC(C)(C)[Si](C)(C)OCCS(=O)(=O)Nc1ccc(-c2ncc(-c3cccc(N4CCC(F)(F)CC4)n3)[nH]2)c(N2CCC3(CC2)CC3)c1] | I04.05 | I03.02 | 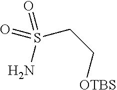 [SMILES: CC(C)(C)[Si](C)(C)OCCS(N)(=O)=O] |

### Example 9. Synthesis of N-(4-(5-(6-(4,4-difluoropiperidin-1-yl)pyridin-2-yl)-1H-imidazol-2-yl)-3-(6-azaspiro[2.5]octan-6-yl)phenyl)-2-hydroxyethane-1-sulfonamide (Compound 35)

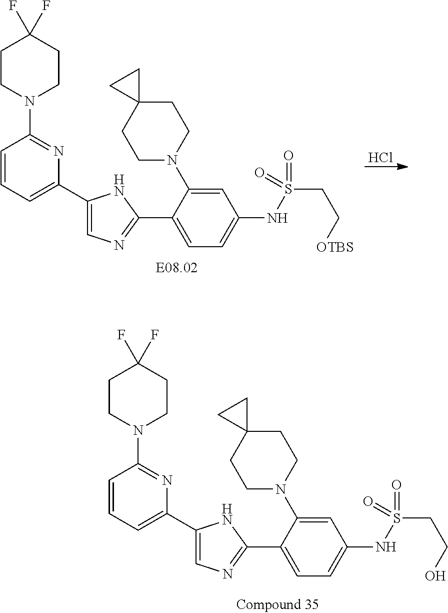 [SMILES: CC(C)(C)[Si](C)(C)OCCS(=O)(=O)Nc1ccc(-c2ncc(-c3cccc(N4CCC(F)(F)CC4)n3)[nH]2)c(N2CCC3(CC2)CC3)c1 ; Cl ; O=S(=O)(CCO)Nc1ccc(-c2ncc(-c3cccc(N4CCC(F)(F)CC4)n3)[nH]2)c(N2CCC3(CC2)CC3)c1]

A mixture of E08.02 (50 mg, 73 μmol), EtOAc (0.5 mL), 6M HCl in EtOAc (12 μL, 73 μmol) was stirred at 25° C. for 1 h. A separate mixture was prepared in the same manner from 10 mg of E08.02. The mixtures were combined, concentrated, combined water (30 mL) and extracted with EtOAc (2×30 mL). The organic phase was washed with brine (10 mL), dried over Na₂SO₄, concentrated, and purified by preparative HPLC (C18; 1-50% MeCN in H₂O [0.1% formic acid]) to provide N-(4-(5-(6-(4,4-difluoropiperidin-1-yl)pyridin-2-yl)-1H-imidazol-2-yl)-3-(6-azaspiro[2.5]octan-6-yl)phenyl)-2-hydroxyethane-1-sulfonamide (Compound 35, 3.8 mg).

### Example 10. Synthesis of N-(4-(4-(6-(4,4-difluoropiperidin-1-yl)pyridin-2-yl)-1H-pyrazol-1-yl)-3-(6-azaspiro[2.5]octan-6-yl)phenyl)methanesulfonamide (Compound 17)

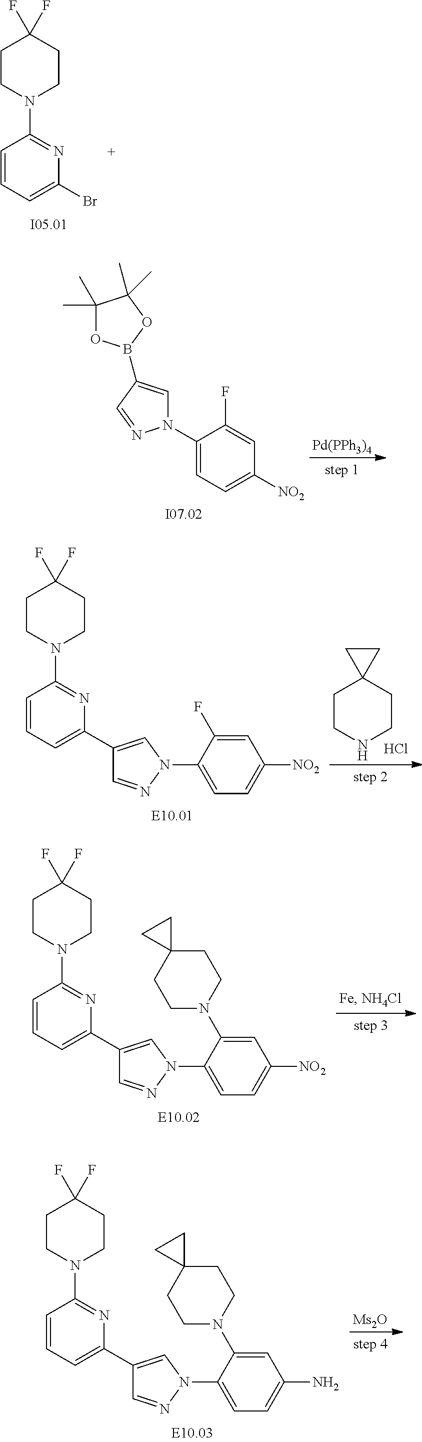

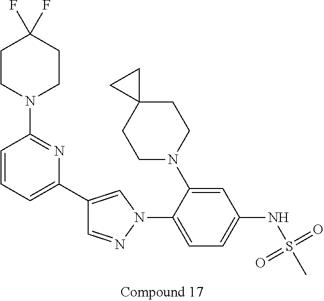 [SMILES: CS(=O)(=O)Nc1ccc(-n2cc(-c3cccc(N4CCC(F)(F)CC4)n3)cn2)c(N2CCC3(CC2)CC3)c1]

Step 1. A mixture of I07.02 (0.55 g, 1.7 mmol), I05.01 (0.55 g, 2.0 mmol), Na₂CO₃ (0.53 mg, 5.0 mmol), H₂O (5 mL), dioxane (15 mL), and Pd(PPh₃)₄(0.19 g, 0.17 mmol) was heated at 90° C. for 2 h under N₂. The mixture was poured into H₂O (20 mL), extracted with EtOAc (10 mL×3), and the combined extracts were washed with brine (20 mL×2), dried over Na₂SO₄, filtered, concentrated, and purified by silica chromatography (0-40%/EtOAc in PE) to provide 2-(4,4-difluoro-1-piperidyl)-6-[1-(2-fluoro-4-nitro-phenyl)pyrazol-4-yl]pyridine (E10.01, 0.47 g).

Step 2. To a mixture of 6-azaspiro[2.5]octane hydrochloride (0.59 g, 4.0 mmol) and DMF (7 mL) was added K₂CO₃ (0.74 g, 5.4 mmol), E10.01 (0.54 g, 1.3 mmol). The mixture was stirred at 120° C. for 16 h, poured into H₂O (10 mL) and EtOAc (10 mL). The resulting precipitate was filtered and dried to provide 6-[2-[4-[6-(4,4-difluoro-1-piperidyl)-2-pyridyl]pyrazol-1-yl]-5-nitro-phenyl]-6-azaspiro[2.5]octane (E10.02, 0.40 g).

Step 3. To a mixture of E10.02 (0.4 g, 0.81 mmol), NH₄C (0.22 g, 4.0 mmol), EtOH (15 mL) and H₂O (7.5 mL) was added Fe (0.45 g, 8.1 mmol). The mixture was heated to 70° C. for 1.5 h. The mixture was filtered through Celite, the filtrate was concentrated, dissolved in EtOAc (10 mL), washed with H₂O (10 mL×2), brine (10 mL), dried over Na₂SO₄, filtered, concentrated, and purified by silica chromatography (0-70% EtOAc in PE) to provide 3-(6-azaspiro[2.5]octan-6-yl)-4-[4-[6-(4,4-difluoro-1-piperidyl)-2-pyridyl]pyrazol-1-yl]aniline (E10.03, 0.36 g).

To a mixture of E10.03 (0.05 g, 0.11 mmol), Et₃N (45 μg, 0.32 mmol), CH₂Cl₂ (1 mL) was added methylsulfonyl methanesulfonate (23 mg, 0.13 mmol) in CH₂Cl₂ (1 mL) dropwise at 0° C. The mixture was stirred at 20° C. for 1.5 h. Additional methylsulfonyl methanesulfonate (23 mg, 0.13 mmol) and Et₃N (45 μg, 0.32 mmol) were added, the mixture was stirred for 4.5 hours at 20° C., then heated to 40° C. for 17 h. The mixture was poured into H₂O (10 mL) and extracted with EtOAc (10 mL×2). The combined extracts were washed with brine (10 mL×3), dried over Na₂SO₄, filtered, concentrated, and purified by preparative HPLC (C18; 40-70% MeCN in H₂O [HCl]) to provide N-(4-(4-(6-(4,4-difluoropiperidin-1-yl)pyridin-2-yl)-1H-pyrazol-1-yl)-3-(6-azaspiro[2.5]octan-6-yl)phenyl)methanesulfonamide (Compound 17, 5.8 mg).

N-(4-(4-(4-(4,4-difluoropiperidin-1-yl)pyrimidin-2-yl)-1H-pyrazol-1-yl)-3-(6-azaspiro[2.5]octan-6-yl)phenyl)methanesulfonamide (Compound 66) was prepared from 107.02 and 2-chloro-4-(4,4-difluoropiperidin-1-yl)pyrimidine in the same manner as Compound 17.

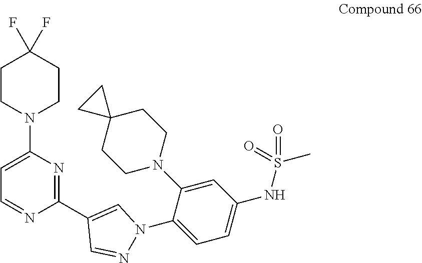 [SMILES: CS(=O)(=O)Nc1ccc(-n2cc(-c3nccc(N4CCC(F)(F)CC4)n3)cn2)c(N2CCC3(CC2)CC3)c1]

### Example 11. Synthesis of N-(5-(4-(5-(cyclopentyl(hydroxy)methyl)furan-2-yl)-1H-1,2,3-triazol-1-yl)-6-(6-azaspiro[2.5]octan-6-yl)pyridin-2-yl)methanesulfonamide (Compound 49)

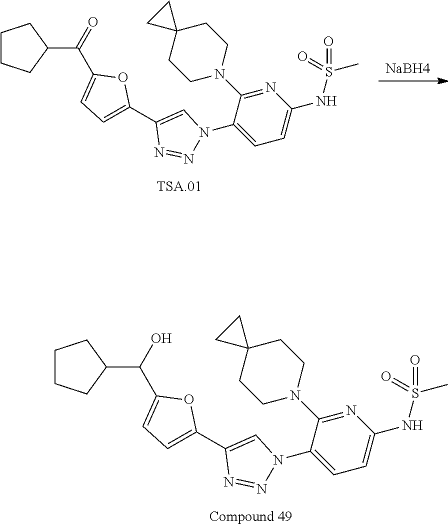 [SMILES: CS(=O)(=O)Nc1ccc(-n2cc(-c3ccc(C(=O)C4CCCC4)o3)nn2)c(N2CCC3(CC2)CC3)n1 ; "NaBH4" ; CS(=O)(=O)Nc1ccc(-n2cc(-c3ccc(C(O)C4CCCC4)o3)nn2)c(N2CCC3(CC2)CC3)n1]

A mixture of T6A.01 (60 mg, 0.12 mmol), MeOH (0.5 mL), and NaBH₄ (9 mg, 0.24 mmol) at 0° C. was stirred at 20° C. for 2 h. The mixture was poured into H₂O (10 mL) and extracted with EtOAc (2×10 mL). The combined extracts were washed with brine (10 mL), dried over Na₂SO₄, concentrated, and purified by preparative HPLC (C18; 40-70% MeCN in H₂O [NH₄HCO₃]) to provide N-(5-(4-(5-(cyclopentyl(hydroxy)methyl)furan-2-yl)-1H-1,2,3-triazol-1-yl)-6-(6-azaspiro[2.5]octan-6-yl)pyridin-2-yl)methanesulfonamide (Compound 49, 12 mg).

Compounds in Table 6C were prepared from the ketone in the same manner as Compound 49.

TABLE 6C

| Name | Structure | Ketone |
|---|---|---|
| Compound 25 | 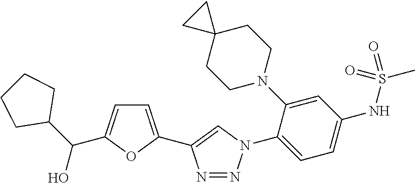 [SMILES: CS(=O)(=O)Nc1ccc(-n2cc(-c3ccc(C(O)C4CCCC4)o3)nn2)c(N2CCC3(CC2)CC3)c1] | T6X.01 |
| Compound 51 | 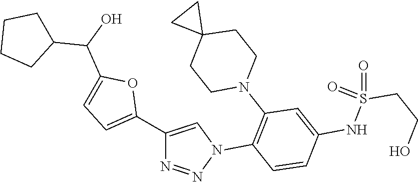 [SMILES: O=S(=O)(CCO)Nc1ccc(-n2cc(-c3ccc(C(O)C4CCCC4)o3)nn2)c(N2CCC3(CC2)CC3)c1] | Compound 92 |
| Compound 123 | 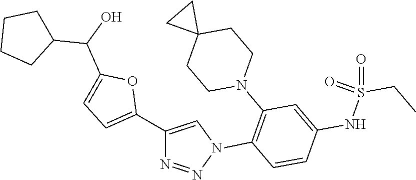 [SMILES: CCS(=O)(=O)Nc1ccc(-n2cc(-c3ccc(C(O)C4CCCC4)o3)nn2)c(N2CCC3(CC2)CC3)c1] | T6A.06 |

### Example 12. Synthesis of N-(4-(4-(6-morpholinopyridin-2-yl)-1H-1,2,3-triazol-1-yl)-3-(6-azaspiro[2.5]octan-6-yl)phenyl)methanesulfonamide (Compound 54)

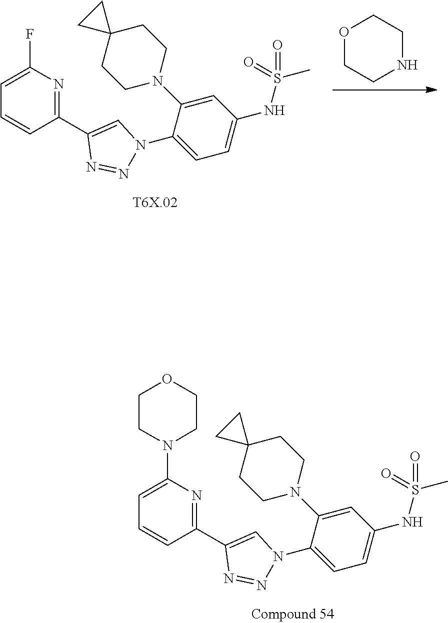

A mixture of T6X.02 (90 mg, 0.20 mmol), morpholine (0.14 mL, 1.6 mmol), DMF (5 mL), and K₂CO₃ (84 mg, 0.61 mmol) was stirred at 120° C. for 12 h. The mixture was poured into H₂O (30 mL) and extracted with EtOAc (2×30 mL). The combined extracts were washed with brine (10 mL), dried over Na₂SO₄, filtered, concentrated, and purified by preparative HPLC (C18; 45-60% MeCN in water [formic acid[) to provide N-(4-(4-(6-morpholinopyridin-2-yl)-1H-1,2,3-triazol-1-yl)-3-(6-azaspiro[2.5]octan-6-yl)phenyl)methanesulfonamide (Compound 54; 14 mg).

Compounds in Table 6D were prepared from the indicated aryl fluoride and the indicated nucleophile in the same manner as Compound 54.

TABLE 6D

| Name | Structure | Fluoride | Nucleophile |
|---|---|---|---|
| Compound 55 | 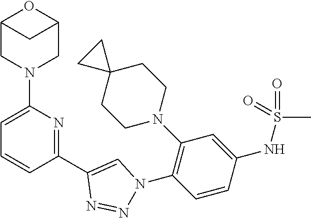 [SMILES: CS(=O)(=O)Nc1ccc(-n2cc(-c3cccc(N4CC5CC(C4)O5)n3)nn2)c(N2CCC3(CC2)CC3)c1] | T6X.02 | 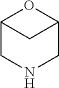 |
| Compound 56 | 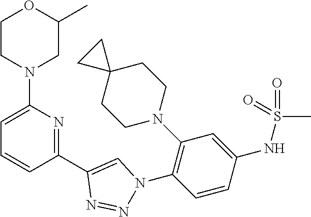 [SMILES: CC1CN(c2cccc(-c3cn(-c4ccc(NS(C)(=O)=O)cc4N4CCC5(CC4)CC5)nn3)n2)CCO1] | T6X.02 | 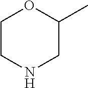 |
| Compound 57 | 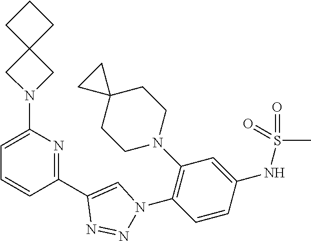 [SMILES: CS(=O)(=O)Nc1ccc(-n2cc(-c3cccc(N4CC5(CCC5)C4)n3)nn2)c(N2CCC3(CC2)CC3)c1] | T6X.02 | 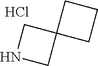 |
| Compound 58 | 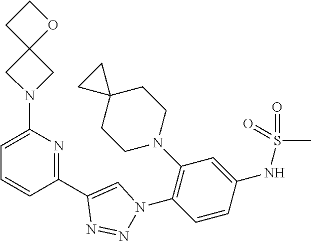 [SMILES: CS(=O)(=O)Nc1ccc(-n2cc(-c3cccc(N4CC5(CCO5)C4)n3)nn2)c(N2CCC3(CC2)CC3)c1] | T6X.02 | 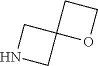 |
| Compound 60 | 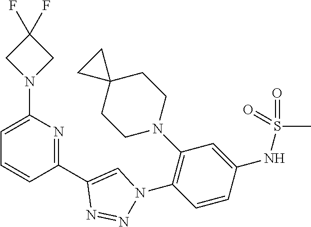 [SMILES: CS(=O)(=O)Nc1ccc(-n2cc(-c3cccc(N4CC(F)(F)C4)n3)nn2)c(N2CCC3(CC2)CC3)c1] | T6X.02 | 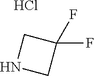 |

### Example 13 Synthesis of N-(5-(4-(6-(4,4-difluoropiperidin-1-yl)pyridin-2-yl)-1H-1,2,3-triazol-1-yl)-4-(6-azaspiro[2.5]octan-6-yl)pyridin-2-yl)methanesulfonamide (Compound 59)

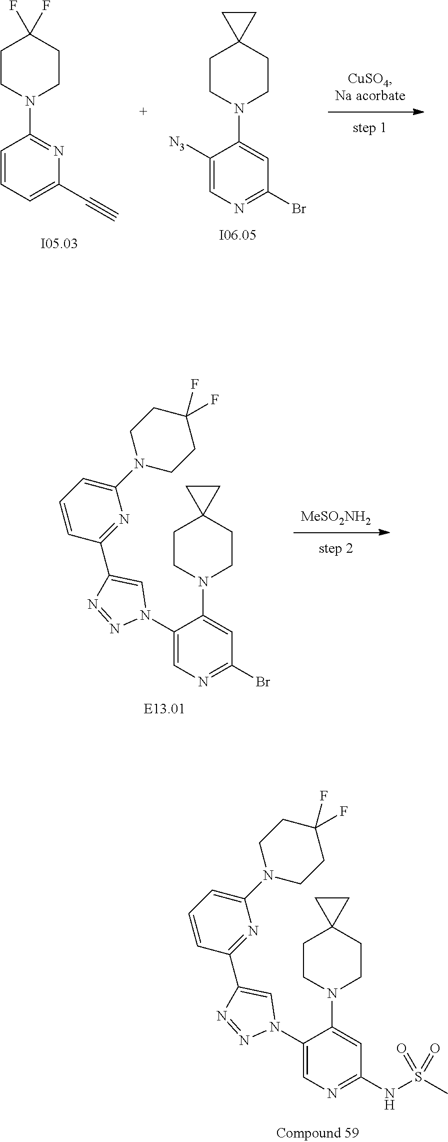

Step 1. Two mixtures of I05.03 (0.12 g, 0.54 mmol & 0.05 g, 0.23 mmol), I06.05 (0.20 g, 0.65 mmol & 0.08 g, 0.27 mmol), CH₂Cl₂ (2 mL & 0.85 mL), H₂O (2 mL & 0.85 mL), sodium ascorbate (0.11 g, 0.54 mmol & 0.045 g, 0.23 mmol), and CuSO₄·5H₂O (14 mg, 54 μmol & 5.8 mg, 23 μmol) were stirred at 20° C. for 2.5 h. The reaction mixtures were combined, poured into H₂O (10 mL), and extracted with EtOAc (2×10 mL). The combined extracts were washed with brine (10 mL), dried over Na₂SO₄, filtered, concentrated, and purified by silica chromatography (0-50% EtOAc in PE) to provide 6-[2-bromo-5-[4-[6-(4,4-difluoro-1-piperidyl)-2-pyridyl]triazol-1-yl]-4-pyridyl]-6-azaspiro[2.5]octane (E13.01, 0.20 g).

Step 2. Two mixtures of methanesulfonamide (38 mg, 0.40 mmol &), E13.01 (70 mg, 0.13 mmol & 20 mg, 0.04 mmol), (1R,2R)—N1,N2-dimethylcyclohexane1,2-diamine (11 mg, 79 μmol & 3.1 mg, 23 μmol), DMF (0.5 mL & 0.15 mL), CuI (15 mg, 79 μmol & 4.3 mg, 23 μmol), and K₃PO₄ (84 mg, 0.40 mmol & 24 mg, 0.11 mmol) were stirred at 140° C. for 2 h. The mixtures were combined, poured into H₂O (30 mL), and extracted with EtOAc (2×30 mL). The combined extracts were washed with brine (10 mL), dried over Na₂SO₄, concentrated, and purified by preparative HPLC (C18; 35-65% MeCN in H₂O [formic acid]) to provide N-(5-(4-(6-(4,4-difluoropiperidin-1-yl)pyridin-2-yl)-1H-1,2,3-triazol-1-yl)-4-(6-azaspiro[2.5]octan-6-yl)pyridin-2-yl)methanesulfonamide (Compound 59; 25 mg).

Compounds in Table 6E were prepared from the indicated azide, alkyne, sulfonamide in the same manner as Compound 59

TABLE 6E

| Name | Structure | Azide | Alkyne | Sulfonamide |
|---|---|---|---|---|
| Compound 46 | 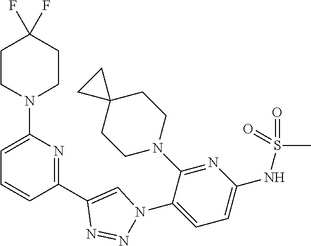 [SMILES: CS(=O)(=O)Nc1ccc(-n2cc(-c3cccc(N4CCC(F)(F)CC4)n3)nn2)c(N2CCC3(CC2)CC3)n1] | I06.04 | I05.03 | 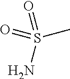 |
| T6A.01 | 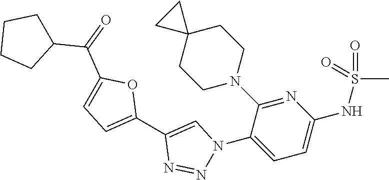 [SMILES: CS(=O)(=O)Nc1ccc(-n2cc(-c3ccc(C(=O)C4CCCC4)o3)nn2)c(N2CCC3(CC2)CC3)n1] | I06.04 | I05.19 | 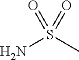 |
| Compound 64 | 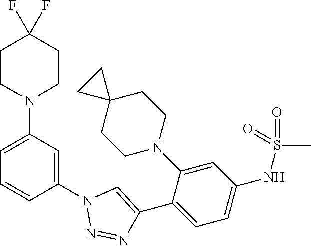 [SMILES: CS(=O)(=O)Nc1ccc(-c2cn(-c3cccc(N4CCC(F)(F)CC4)c3)nn2)c(N2CCC3(CC2)CC3)c1] | I09.03 | I05.20 | 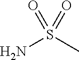 |
| Compound 65 | 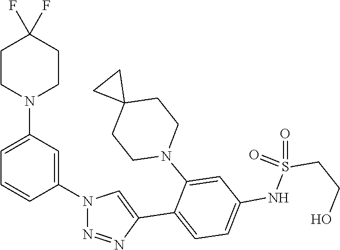 [SMILES: O=S(=O)(CCO)Nc1ccc(-c2cn(-c3cccc(N4CCC(F)(F)CC4)c3)nn2)c(N2CCC3(CC2)CC3)c1] | I09.03 | I05.20 | 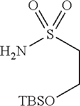 [SMILES: CC(C)(C)[Si](C)(C)OCCS(N)(=O)=O] |
| Compound 67 | 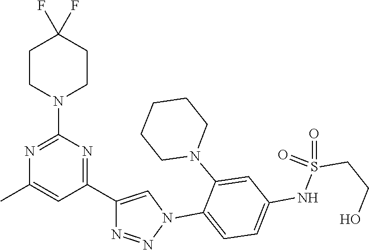 [SMILES: Cc1cc(-c2cn(-c3ccc(NS(=O)(=O)CCO)cc3N3CCCCC3)nn2)nc(N2CCC(F)(F)CC2)n1] | I06.11 | I05.11 | 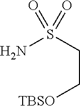 [SMILES: CC(C)(C)[Si](C)(C)OCCS(N)(=O)=O] |
| Compound 68 | 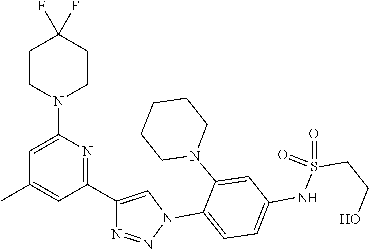 [SMILES: Cc1cc(-c2cn(-c3ccc(NS(=O)(=O)CCO)cc3N3CCCCC3)nn2)nc(N2CCC(F)(F)CC2)c1] | I06.11 | I05.08 | 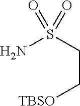 [SMILES: CC(C)(C)[Si](C)(C)OCCS(N)(=O)=O] |
| Compound 72 | 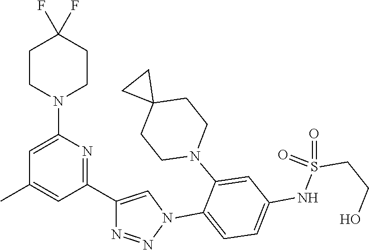 [SMILES: Cc1cc(-c2cn(-c3ccc(NS(=O)(=O)CCO)cc3N3CCC4(CC3)CC4)nn2)nc(N2CCC(F)(F)CC2)c1] | I06.06 | I05.08 | 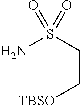 [SMILES: CC(C)(C)[Si](C)(C)OCCS(N)(=O)=O] |
| Compound 73 | 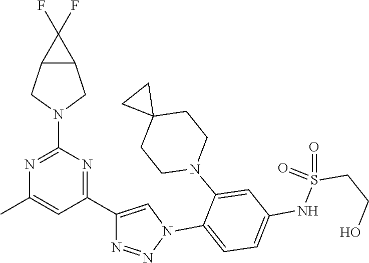 [SMILES: Cc1cc(-c2cn(-c3ccc(NS(=O)(=O)CCO)cc3N3CCC4(CC3)CC4)nn2)nc(N2CC3C(C2)C3(F)F)n1] | I06.06 | I05.23 | 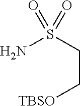 [SMILES: CC(C)(C)[Si](C)(C)OCCS(N)(=O)=O] |
| Compound 74 | 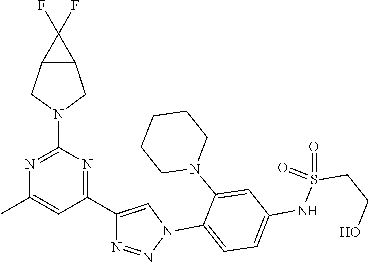 [SMILES: Cc1cc(-c2cn(-c3ccc(NS(=O)(=O)CCO)cc3N3CCCCC3)nn2)nc(N2CC3C(C2)C3(F)F)n1] | I06.11 | I05.23 | 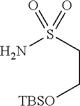 [SMILES: CC(C)(C)[Si](C)(C)OCCS(N)(=O)=O] |
| Compound 75 | 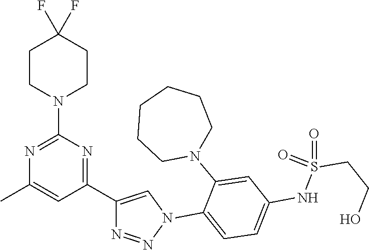 [SMILES: Cc1cc(-c2cn(-c3ccc(NS(=O)(=O)CCO)cc3N3CCCCCC3)nn2)nc(N2CCC(F)(F)CC2)n1] | I06.12 | I05.11 | 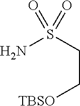 [SMILES: CC(C)(C)[Si](C)(C)OCCS(N)(=O)=O] |
| Compound 76 | 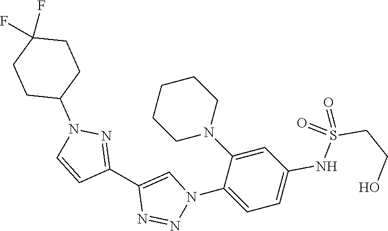 [SMILES: O=S(=O)(CCO)Nc1ccc(-n2cc(-c3ccn(C4CCC(F)(F)CC4)n3)nn2)c(N2CCCCC2)c1] | I06.11 | I05.24 | 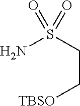 [SMILES: CC(C)(C)[Si](C)(C)OCCS(N)(=O)=O] |
| Compound 77 | 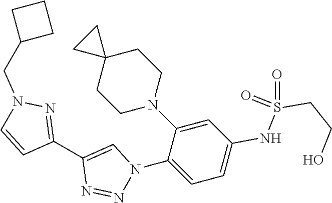 [SMILES: O=S(=O)(CCO)Nc1ccc(-n2cc(-c3ccn(CC4CCC4)n3)nn2)c(N2CCC3(CC2)CC3)c1] | I06.06 | I05.25 | 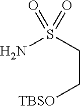 [SMILES: CC(C)(C)[Si](C)(C)OCCS(N)(=O)=O] |
| Compound 78 | 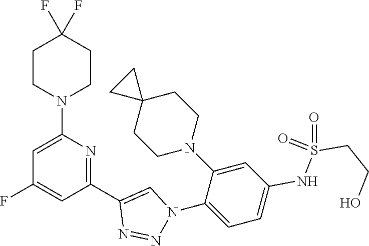 [SMILES: O=S(=O)(CCO)Nc1ccc(-n2cc(-c3cc(F)cc(N4CCC(F)(F)CC4)n3)nn2)c(N2CCC3(CC2)CC3)c1] | I06.06 | I05.26 | 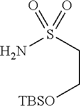 [SMILES: CC(C)(C)[Si](C)(C)OCCS(N)(=O)=O] |
| Compound 79 | 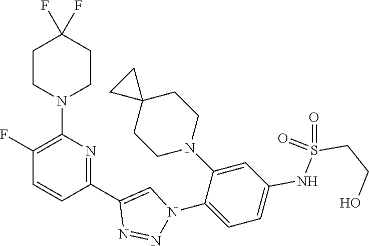 [SMILES: O=S(=O)(CCO)Nc1ccc(-n2cc(-c3ccc(F)c(N4CCC(F)(F)CC4)n3)nn2)c(N2CCC3(CC2)CC3)c1] | I06.06 | I05.27 | 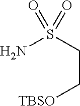 [SMILES: CC(C)(C)[Si](C)(C)OCCS(N)(=O)=O] |
| Compound 80 | 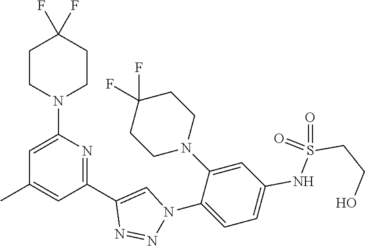 [SMILES: Cc1cc(-c2cn(-c3ccc(NS(=O)(=O)CCO)cc3N3CCC(F)(F)CC3)nn2)nc(N2CCC(F)(F)CC2)c1] | I06.13 | I05.08 | 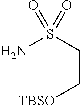 [SMILES: CC(C)(C)[Si](C)(C)OCCS(N)(=O)=O] |
| Compound 81 | 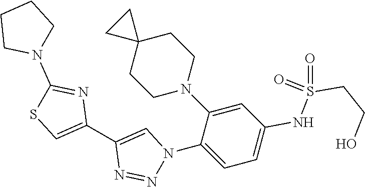 [SMILES: O=S(=O)(CCO)Nc1ccc(-n2cc(-c3csc(N4CCCC4)n3)nn2)c(N2CCC3(CC2)CC3)c1] | I06.06 | I05.28 | 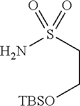 [SMILES: CC(C)(C)[Si](C)(C)OCCS(N)(=O)=O] |
| Compound 82 | 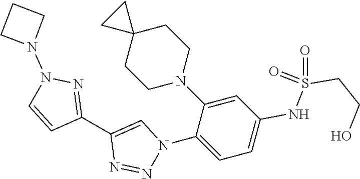 [SMILES: O=S(=O)(CCO)Nc1ccc(-n2cc(-c3ccn(N4CCC4)n3)nn2)c(N2CCC3(CC2)CC3)c1] | I06.06 | I05.29 | 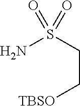 [SMILES: CC(C)(C)[Si](C)(C)OCCS(N)(=O)=O] |
| Compound 83 | 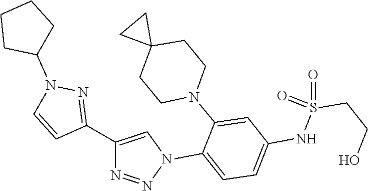 [SMILES: O=S(=O)(CCO)Nc1ccc(-n2cc(-c3ccn(C4CCCC4)n3)nn2)c(N2CCC3(CC2)CC3)c1] | I06.06 | I05.30 | 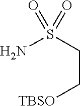 [SMILES: CC(C)(C)[Si](C)(C)OCCS(N)(=O)=O] |
| Compound 84 | 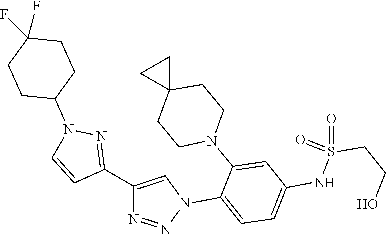 [SMILES: O=S(=O)(CCO)Nc1ccc(-n2cc(-c3ccn(C4CCC(F)(F)CC4)n3)nn2)c(N2CCC3(CC2)CC3)c1] | I06.06 | I05.24 | 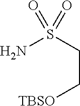 [SMILES: CC(C)(C)[Si](C)(C)OCCS(N)(=O)=O] |
| Compound 86 | 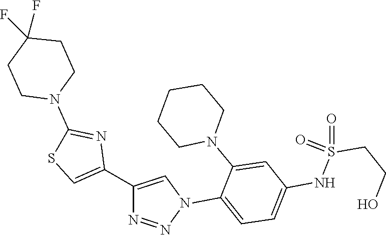 [SMILES: O=S(=O)(CCO)Nc1ccc(-n2cc(-c3csc(N4CCC(F)(F)CC4)n3)nn2)c(N2CCCCC2)c1] | I06.01 | R-038 | 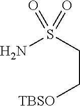 [SMILES: CC(C)(C)[Si](C)(C)OCCS(N)(=O)=O] |
| Compound 87 | 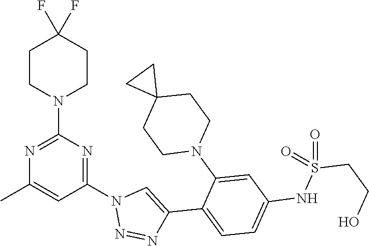 [SMILES: Cc1cc(-n2cc(-c3ccc(NS(=O)(=O)CCO)cc3N3CCC4(CC3)CC4)nn2)nc(N2CCC(F)(F)CC2)n1] | I06.16 | I05.20 | 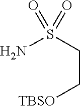 [SMILES: CC(C)(C)[Si](C)(C)OCCS(N)(=O)=O] |
| Compound 88 | 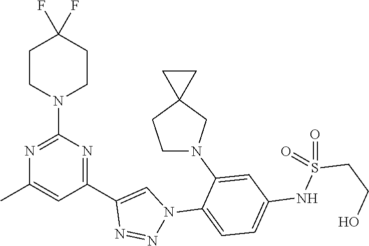 [SMILES: Cc1cc(-c2cn(-c3ccc(NS(=O)(=O)CCO)cc3N3CCC4(CC4)C3)nn2)nc(N2CCC(F)(F)CC2)n1] | I06.18 | I05.11 | 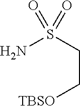 [SMILES: CC(C)(C)[Si](C)(C)OCCS(N)(=O)=O] |
| Compound 89 | 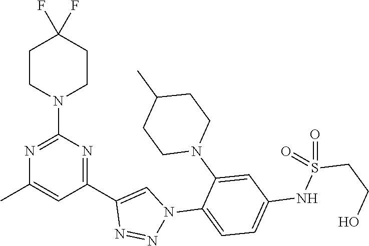 [SMILES: Cc1cc(-c2cn(-c3ccc(NS(=O)(=O)CCO)cc3N3CCC(C)CC3)nn2)nc(N2CCC(F)(F)CC2)n1] | I06.17 | I05.11 | 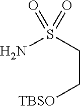 [SMILES: CC(C)(C)[Si](C)(C)OCCS(N)(=O)=O] |
| Compound 90 | 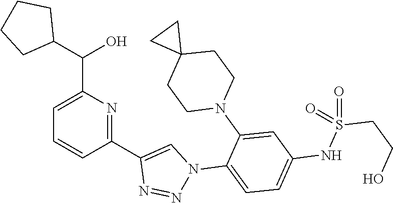 [SMILES: O=S(=O)(CCO)Nc1ccc(-n2cc(-c3cccc(C(O)C4CCCC4)n3)nn2)c(N2CCC3(CC2)CC3)c1] | I06.06 | I05.16 | 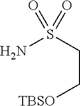 [SMILES: CC(C)(C)[Si](C)(C)OCCS(N)(=O)=O] |
| T6A.05 | 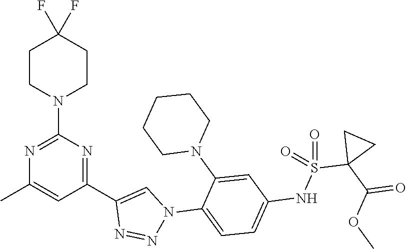 [SMILES: COC(=O)C1(S(=O)(=O)Nc2ccc(-n3cc(-c4cc(C)nc(N5CCC(F)(F)CC5)n4)nn3)c(N3CCCCC3)c2)CC1] | I06.11 | I05.11 | 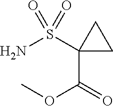 |
| Compound 94 | 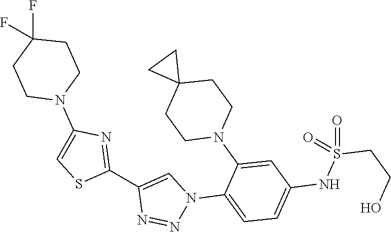 [SMILES: O=S(=O)(CCO)Nc1ccc(-n2cc(-c3nc(N4CCC(F)(F)CC4)cs3)nn2)c(N2CCC3(CC2)CC3)c1] | I06.06 | I05.31 | 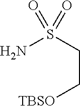 [SMILES: CC(C)(C)[Si](C)(C)OCCS(N)(=O)=O] |
| Compound 95 | 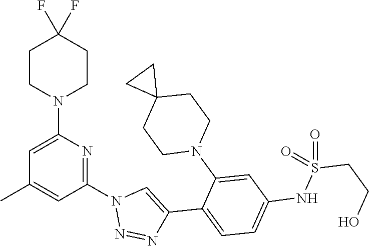 [SMILES: Cc1cc(N2CCC(F)(F)CC2)nc(-n2cc(-c3ccc(NS(=O)(=O)CCO)cc3N3CCC4(CC3)CC4)nn2)c1] | I06.22 | I05.20 | 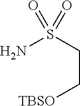 [SMILES: CC(C)(C)[Si](C)(C)OCCS(N)(=O)=O] |
| Compound 96 | 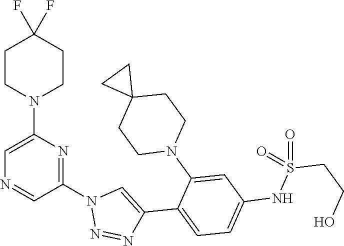 [SMILES: O=S(=O)(CCO)Nc1ccc(-c2cn(-c3cncc(N4CCC(F)(F)CC4)n3)nn2)c(N2CCC3(CC2)CC3)c1] | I06.24 | I05.20 | 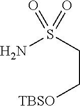 [SMILES: CC(C)(C)[Si](C)(C)OCCS(N)(=O)=O] |
| Compound 97 | 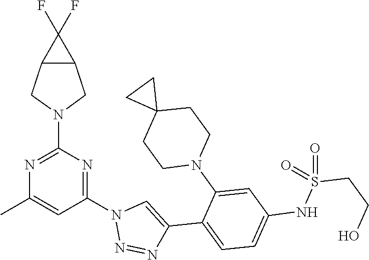 [SMILES: Cc1cc(-n2cc(-c3ccc(NS(=O)(=O)CCO)cc3N3CCC4(CC3)CC4)nn2)nc(N2CC3C(C2)C3(F)F)n1] | I06.25 | I05.20 | 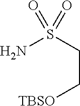 [SMILES: CC(C)(C)[Si](C)(C)OCCS(N)(=O)=O] |
| Compound 98 | 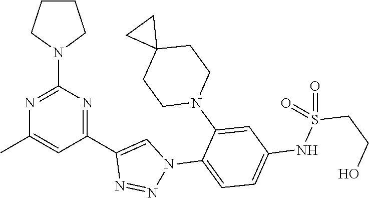 [SMILES: Cc1cc(-c2cn(-c3ccc(NS(=O)(=O)CCO)cc3N3CCC4(CC3)CC4)nn2)nc(N2CCCC2)n1] | I06.06 | I05.33 | 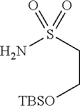 [SMILES: CC(C)(C)[Si](C)(C)OCCS(N)(=O)=O] |
| Compound 99 | 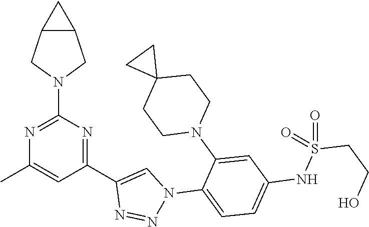 [SMILES: Cc1cc(-c2cn(-c3ccc(NS(=O)(=O)CCO)cc3N3CCC4(CC3)CC4)nn2)nc(N2CC3CC3C2)n1] | I06.06 | I05.33 | 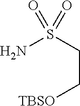 [SMILES: CC(C)(C)[Si](C)(C)OCCS(N)(=O)=O] |
| Compound 101 | 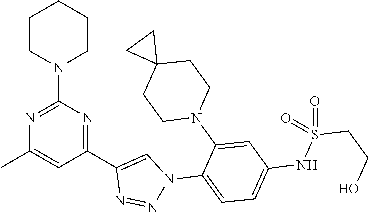 [SMILES: Cc1cc(-c2cn(-c3ccc(NS(=O)(=O)CCO)cc3N3CCC4(CC3)CC4)nn2)nc(N2CCCCC2)n1] | I06.06 | I05.34 | 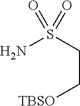 [SMILES: CC(C)(C)[Si](C)(C)OCCS(N)(=O)=O] |
| Compound 102 | 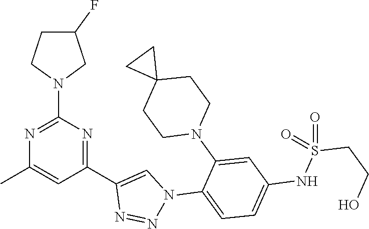 [SMILES: Cc1cc(-c2cn(-c3ccc(NS(=O)(=O)CCO)cc3N3CCC4(CC3)CC4)nn2)nc(N2CCC(F)C2)n1] | I06.06 | I05.35 | 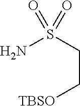 [SMILES: CC(C)(C)[Si](C)(C)OCCS(N)(=O)=O] |
| Compound 103 | 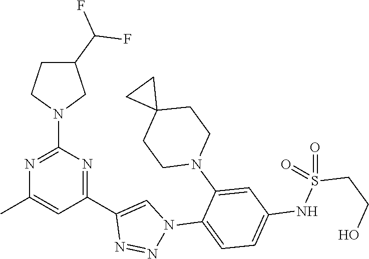 [SMILES: Cc1cc(-c2cn(-c3ccc(NS(=O)(=O)CCO)cc3N3CCC4(CC3)CC4)nn2)nc(N2CCC(C(F)F)C2)n1] | I06.06 | I05.36 | 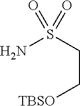 [SMILES: CC(C)(C)[Si](C)(C)OCCS(N)(=O)=O] |
| Compound 104 | 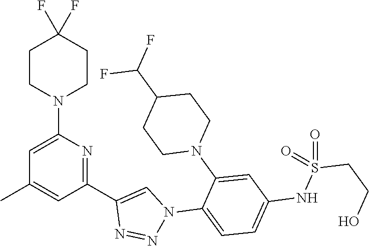 [SMILES: Cc1cc(-c2cn(-c3ccc(NS(=O)(=O)CCO)cc3N3CCC(C(F)F)CC3)nn2)nc(N2CCC(F)(F)CC2)c1] | I06.26 | I05.08 | 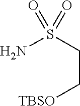 [SMILES: CC(C)(C)[Si](C)(C)OCCS(N)(=O)=O] |
| Compound 105 | 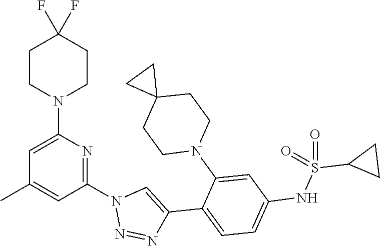 [SMILES: Cc1cc(N2CCC(F)(F)CC2)nc(-n2cc(-c3ccc(NS(=O)(=O)C4CC4)cc3N3CCC4(CC3)CC4)nn2)c1] | I06.22 | I05.20 | 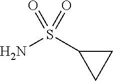 |
| Compound 108 | 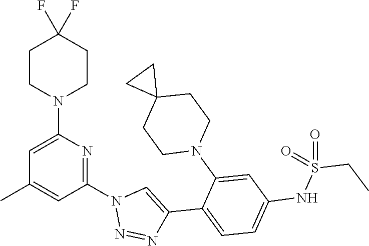 [SMILES: CCS(=O)(=O)Nc1ccc(-c2cn(-c3cc(C)cc(N4CCC(F)(F)CC4)n3)nn2)c(N2CCC3(CC2)CC3)c1] | I06.22 | I05.20 | 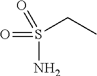 |
| Compound 109 | 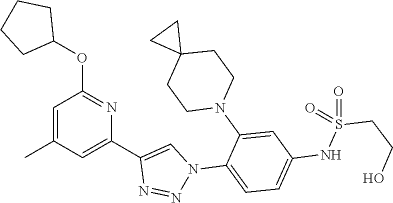 [SMILES: Cc1cc(OC2CCCC2)nc(-c2cn(-c3ccc(NS(=O)(=O)CCO)cc3N3CCC4(CC3)CC4)nn2)c1] | I06.06 | I05.37 | 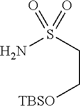 [SMILES: CC(C)(C)[Si](C)(C)OCCS(N)(=O)=O] |
| Compound 110 | 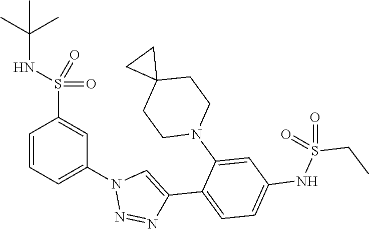 [SMILES: CCS(=O)(=O)Nc1ccc(-c2cn(-c3cccc(S(=O)(=O)NC(C)(C)C)c3)nn2)c(N2CCC3(CC2)CC3)c1] | I09.04 | I05.20 | 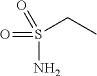 |
| Compound 111 | 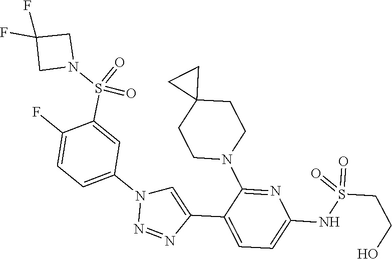 [SMILES: O=S(=O)(CCO)Nc1ccc(-c2cn(-c3ccc(F)c(S(=O)(=O)N4CC(F)(F)C4)c3)nn2)c(N2CCC3(CC2)CC3)n1] | I09.07 | I05.42 | 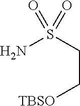 [SMILES: CC(C)(C)[Si](C)(C)OCCS(N)(=O)=O] |
| Compound 112 | 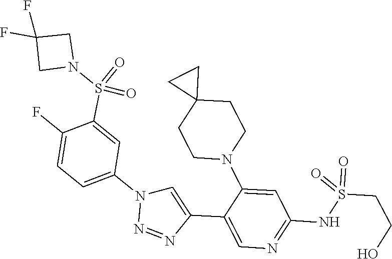 [SMILES: O=S(=O)(CCO)Nc1cc(N2CCC3(CC2)CC3)c(-c2cn(-c3ccc(F)c(S(=O)(=O)N4CC(F)(F)C4)c3)nn2)cn1] | I09.07 | I05.43 | 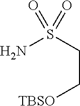 [SMILES: CC(C)(C)[Si](C)(C)OCCS(N)(=O)=O] |
| Compound 117 | 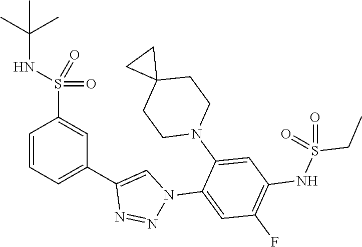 [SMILES: CCS(=O)(=O)Nc1cc(N2CCC3(CC2)CC3)c(-n2cc(-c3cccc(S(=O)(=O)NC(C)(C)C)c3)nn2)cc1F] | I06.27 | I05.13 | 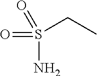 |
| Compound 118 | 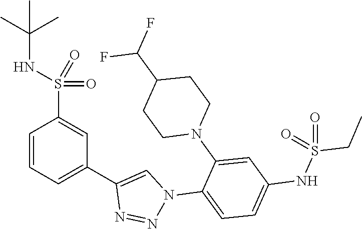 [SMILES: CCS(=O)(=O)Nc1ccc(-n2cc(-c3cccc(S(=O)(=O)NC(C)(C)C)c3)nn2)c(N2CCC(C(F)F)CC2)c1] | I06.26 | I05.13 | 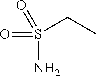 |
| Compound 119 | 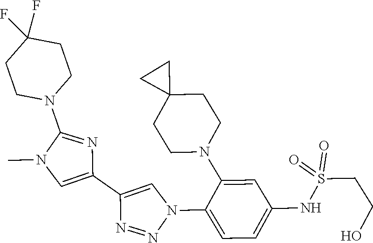 [SMILES: Cn1cc(-c2cn(-c3ccc(NS(=O)(=O)CCO)cc3N3CCC4(CC3)CC4)nn2)nc1N1CCC(F)(F)CC1] | I06.06 | I05.46 | 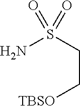 [SMILES: CC(C)(C)[Si](C)(C)OCCS(N)(=O)=O] |
| Compound 120 | 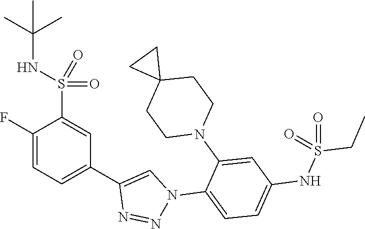 [SMILES: CCS(=O)(=O)Nc1ccc(-n2cc(-c3ccc(F)c(S(=O)(=O)NC(C)(C)C)c3)nn2)c(N2CCC3(CC2)CC3)c1] | I06.06 | I05.47 | 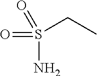 |
| Compound 121 | 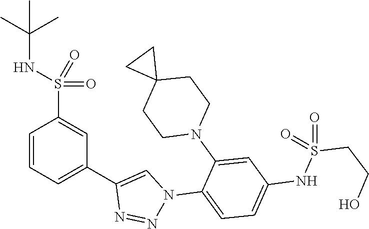 [SMILES: CC(C)(C)NS(=O)(=O)c1cccc(-c2cn(-c3ccc(NS(=O)(=O)CCO)cc3N3CCC4(CC3)CC4)nn2)c1] | I06.06 | I05.13 | 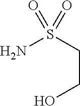 |
| T6A.06 | 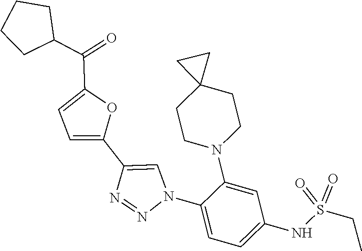 [SMILES: CCS(=O)(=O)Nc1ccc(-n2cc(-c3ccc(C(=O)C4CCCC4)o3)nn2)c(N2CCC3(CC2)CC3)c1] | I06.06 | I05.19 | 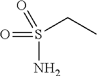 |
| Compound 124 | 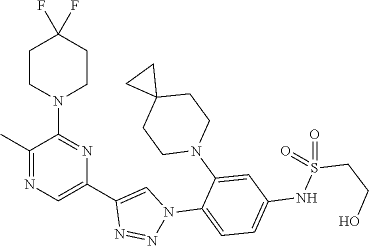 [SMILES: Cc1ncc(-c2cn(-c3ccc(NS(=O)(=O)CCO)cc3N3CCC4(CC3)CC4)nn2)nc1N1CCC(F)(F)CC1] | I06.06 | I05.50 | 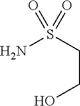 |
| Compound 125 | 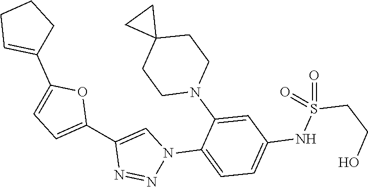 [SMILES: O=S(=O)(CCO)Nc1ccc(-n2cc(-c3ccc(C4=CCCC4)o3)nn2)c(N2CCC3(CC2)CC3)c1] | I06.06 | I05.52 | 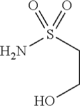 |
| Compound 127A | 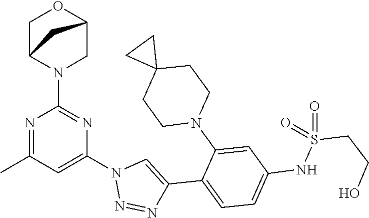 [SMILES: Cc1cc(-n2cc(-c3ccc(NS(=O)(=O)CCO)cc3N3CCC4(CC3)CC4)nn2)nc(N2C[C@@H]3C[C@H]2CO3)n1] | I06.28 | I05.20 | 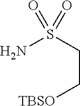 [SMILES: CC(C)(C)[Si](C)(C)OCCS(N)(=O)=O] |
| Compound 127B | 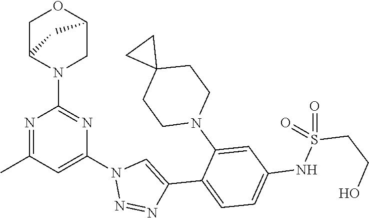 | I06.29 | I05.20 | 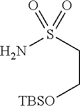 [SMILES: CC(C)(C)[Si](C)(C)OCCS(N)(=O)=O] |
| Compound 128 | 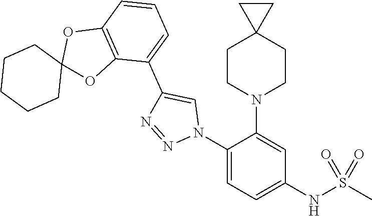 [SMILES: CS(=O)(=O)Nc1ccc(-n2cc(-c3cccc4c3OC3(CCCCC3)O4)nn2)c(N2CCC3(CC2)CC3)c1] | I06.06 | I05.53 | 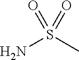 |
| Compound 129 | 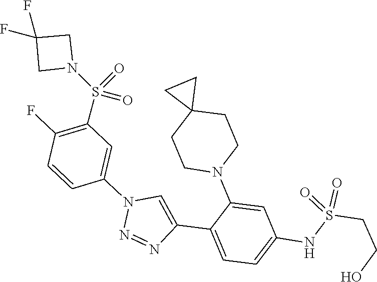 [SMILES: O=S(=O)(CCO)Nc1ccc(-c2cn(-c3ccc(F)c(S(=O)(=O)N4CC(F)(F)C4)c3)nn2)c(N2CCC3(CC2)CC3)c1] | I09.07 | I05.20 | 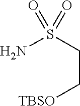 [SMILES: CC(C)(C)[Si](C)(C)OCCS(N)(=O)=O] |
| T6A.07 | 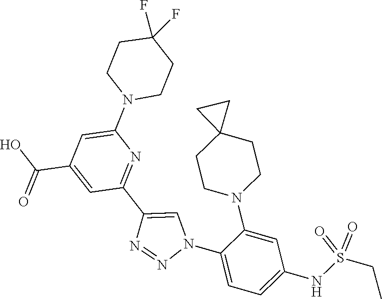 [SMILES: CCS(=O)(=O)Nc1ccc(-n2cc(-c3cc(C(=O)O)cc(N4CCC(F)(F)CC4)n3)nn2)c(N2CCC3(CC2)CC3)c1] | I06.06 | I05.54 | 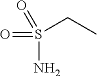 |
| Compound 132 | 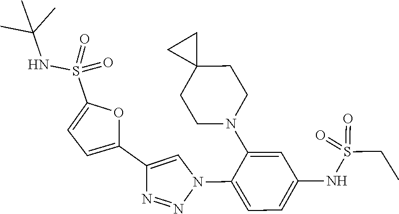 [SMILES: CCS(=O)(=O)Nc1ccc(-n2cc(-c3ccc(S(=O)(=O)NC(C)(C)C)o3)nn2)c(N2CCC3(CC2)CC3)c1] | I06.06 | I05.57 | 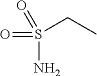 |
| Compound 133 | 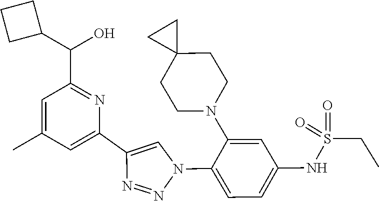 [SMILES: CCS(=O)(=O)Nc1ccc(-n2cc(-c3cc(C)cc(C(O)C4CCC4)n3)nn2)c(N2CCC3(CC2)CC3)c1] | I06.06 | I05.58 | 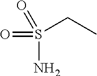 |
| Compound 134 | 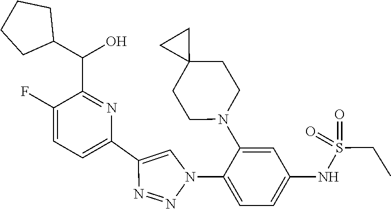 [SMILES: CCS(=O)(=O)Nc1ccc(-n2cc(-c3ccc(F)c(C(O)C4CCCC4)n3)nn2)c(N2CCC3(CC2)CC3)c1] | I06.06 | I05.59 | 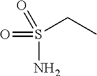 |
| Compound 135 | 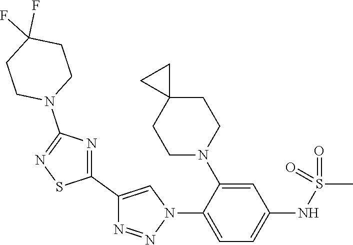 [SMILES: CS(=O)(=O)Nc1ccc(-n2cc(-c3nc(N4CCC(F)(F)CC4)ns3)nn2)c(N2CCC3(CC2)CC3)c1] | I06.06 | I05.60 | 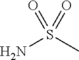 |
| Compound 136 | 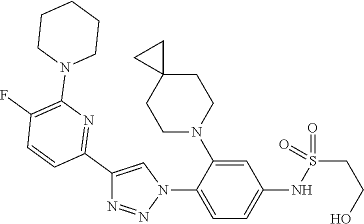 [SMILES: O=S(=O)(CCO)Nc1ccc(-n2cc(-c3ccc(F)c(N4CCCCC4)n3)nn2)c(N2CCC3(CC2)CC3)c1] | I06.06 | I05.61 | 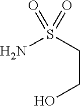 |
| Compound 137 | 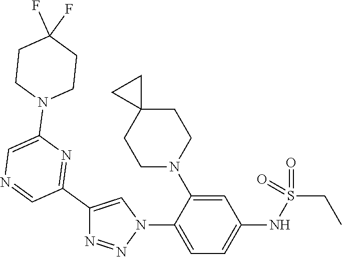 [SMILES: CCS(=O)(=O)Nc1ccc(-n2cc(-c3cncc(N4CCC(F)(F)CC4)n3)nn2)c(N2CCC3(CC2)CC3)c1] | I06.06 | I05.21 | 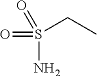 |
| Compound 138 | 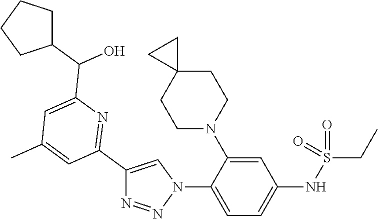 [SMILES: CCS(=O)(=O)Nc1ccc(-n2cc(-c3cc(C)cc(C(O)C4CCCC4)n3)nn2)c(N2CCC3(CC2)CC3)c1] | I06.06 | I05.62 | 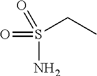 |
| Compound 139 | 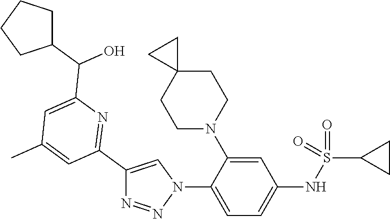 [SMILES: Cc1cc(-c2cn(-c3ccc(NS(=O)(=O)C4CC4)cc3N3CCC4(CC3)CC4)nn2)nc(C(O)C2CCCC2)c1] | I06.06 | I05.62 | 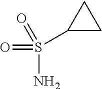 |
| Compound 140 | 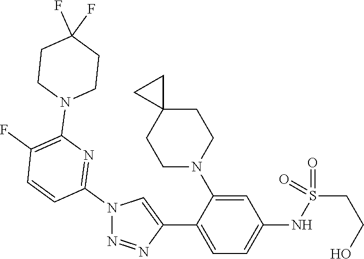 [SMILES: O=S(=O)(CCO)Nc1ccc(-c2cn(-c3ccc(F)c(N4CCC(F)(F)CC4)n3)nn2)c(N2CCC3(CC2)CC3)c1] | I06.31 | I05.20 | 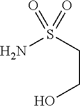 |
| Compound 141 | 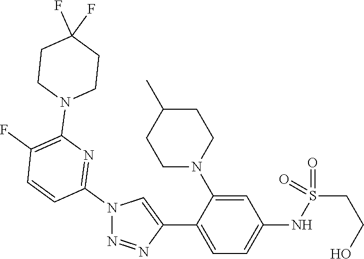 [SMILES: CC1CCN(c2cc(NS(=O)(=O)CCO)ccc2-c2cn(-c3ccc(F)c(N4CCC(F)(F)CC4)n3)nn2)CC1] | I06.31 | I05.63 | 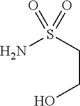 |
| Compound 142 | 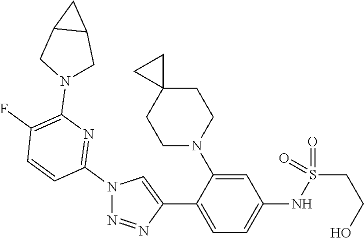 [SMILES: O=S(=O)(CCO)Nc1ccc(-c2cn(-c3ccc(F)c(N4CC5CC5C4)n3)nn2)c(N2CCC3(CC2)CC3)c1] | I06.32 | I05.20 | 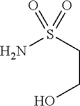 |
| Compound 143 | 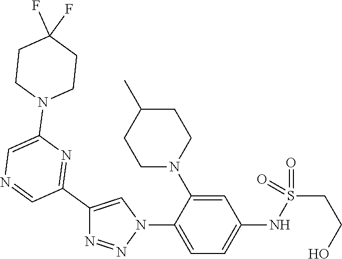 [SMILES: CC1CCN(c2cc(NS(=O)(=O)CCO)ccc2-n2cc(-c3cncc(N4CCC(F)(F)CC4)n3)nn2)CC1] | I06.17 | I05.21 | 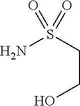 |
| Compound 144 | 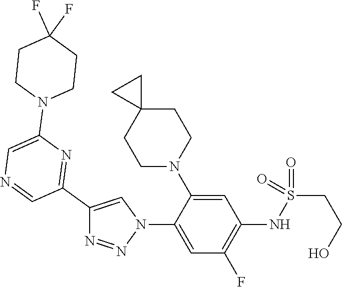 [SMILES: O=S(=O)(CCO)Nc1cc(N2CCC3(CC2)CC3)c(-n2cc(-c3cncc(N4CCC(F)(F)CC4)n3)nn2)cc1F] | I06.27 | I05.21 | 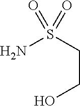 |
| Compound 145 | 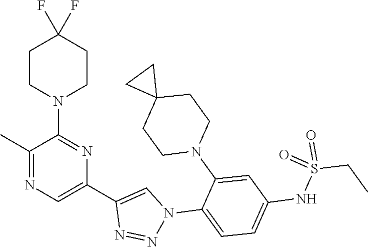 [SMILES: CCS(=O)(=O)Nc1ccc(-n2cc(-c3cnc(C)c(N4CCC(F)(F)CC4)n3)nn2)c(N2CCC3(CC2)CC3)c1] | I06.06 | I05.50 | 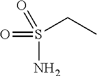 |
| Compound 147 | 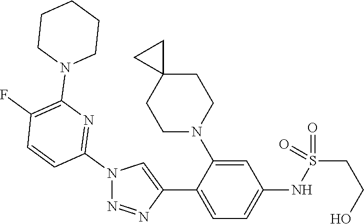 [SMILES: O=S(=O)(CCO)Nc1ccc(-c2cn(-c3ccc(F)c(N4CCCCC4)n3)nn2)c(N2CCC3(CC2)CC3)c1] | I06.32 | I05.20 | 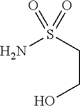 |
| Compound 148 | 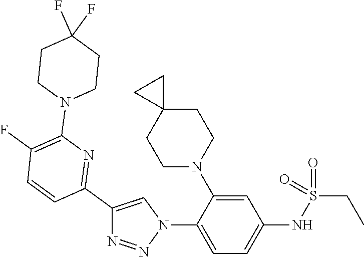 [SMILES: CCS(=O)(=O)Nc1ccc(-n2cc(-c3ccc(F)c(N4CCC(F)(F)CC4)n3)nn2)c(N2CCC3(CC2)CC3)c1] | I06.06 | I05.27 | 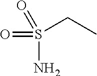 |
| Compound 149 | 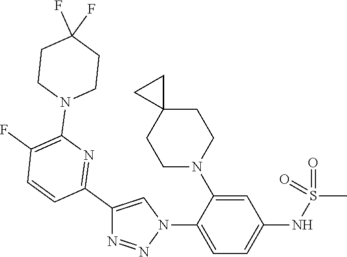 [SMILES: CS(=O)(=O)Nc1ccc(-n2cc(-c3ccc(F)c(N4CCC(F)(F)CC4)n3)nn2)c(N2CCC3(CC2)CC3)c1] | I06.06 | I05.27 | 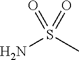 |
| Compound 150 | 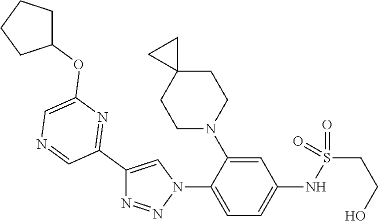 [SMILES: O=S(=O)(CCO)Nc1ccc(-n2cc(-c3cncc(OC4CCCC4)n3)nn2)c(N2CCC3(CC2)CC3)c1] | I06.06 | I05.66 | 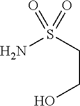 |
| Compound 153 | 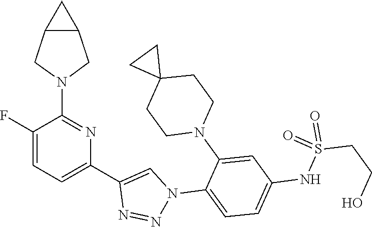 [SMILES: O=S(=O)(CCO)Nc1ccc(-n2cc(-c3ccc(F)c(N4CC5CC5C4)n3)nn2)c(N2CCC3(CC2)CC3)c1] | I06.06 | I05.74 | 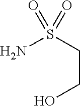 |
| Compound 156 | 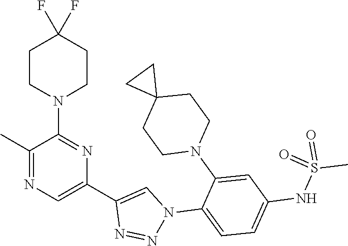 | I06.06 | I05.50 | 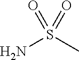 |
| T6A.08 | 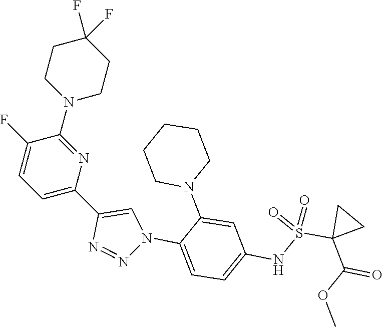 [SMILES: COC(=O)C1(S(=O)(=O)Nc2ccc(-n3cc(-c4ccc(F)c(N5CCC(F)(F)CC5)n4)nn3)c(N3CCCCC3)c2)CC1] | I06.11 | I05.27 | 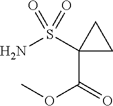 |
| Compound 161 | 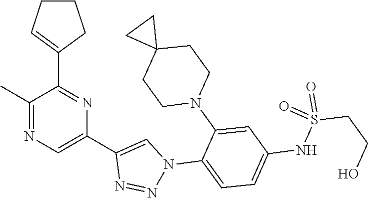 [SMILES: Cc1ncc(-c2cn(-c3ccc(NS(=O)(=O)CCO)cc3N3CCC4(CC3)CC4)nn2)nc1C1=CCCC1] | I06.06 | I05.83 | 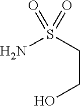 |

### Example 14 Synthesis of N-(4-(l-(6-(4,4-difluoropiperidin-1-yl)pyridin-2-yl)-1H-1,2,3-triazol-4-yl)-3-(6-azaspiro[2.5]octan-6-yl)phenyl)methanesulfonamide (Compound 29)

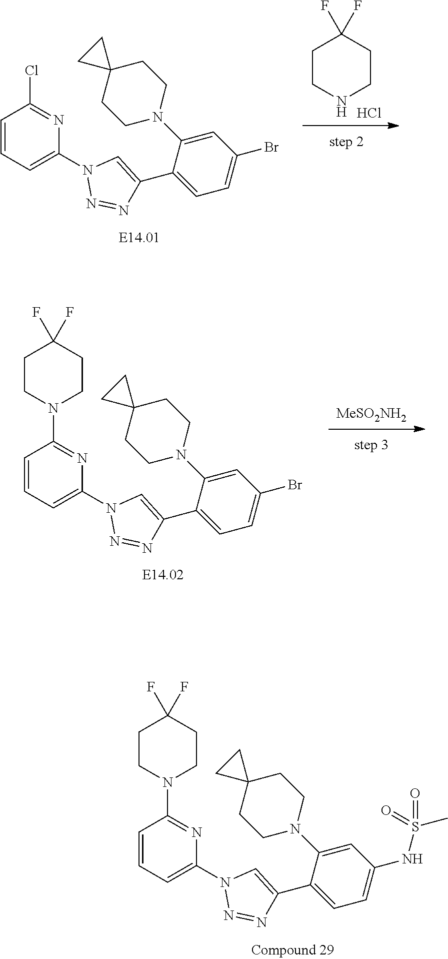 [SMILES: Clc1cccc(-n2cc(-c3ccc(Br)cc3N3CCC4(CC3)CC4)nn2)n1 ; "MeSO2NH2" ; FC1(F)CCNCC1 ; Cl ; FC1(F)CCN(c2cccc(-n3cc(-c4ccc(Br)cc4N4CCC5(CC4)CC5)nn3)n2)CC1 ; CS(=O)(=O)Nc1ccc(-c2cn(-c3cccc(N4CCC(F)(F)CC4)n3)nn2)c(N2CCC3(CC2)CC3)c1]

Step 2. A mixture of E14.01 (0.28 g, 0.63 mmol), 4,4-difluoropiperidine hydrochloride (0.20 g, 1.3 mmol), DMSO (5 mL), and CsF (0.29 mg, 1.9 mmol) was stirred at 120° C. for 12 h. The mixture was poured into H₂O (30 mL), extracted with EtOAc (2×30 mL), and the combined extracts were washed with brine (10 mL), dried over Na₂SO₄, concentrated, and purified by silica chromatography (0-40% EtOAc in PE) to provide the compound 6-(5-bromo-2-(1-(6-(4,4-difluoropiperidin-1-yl)pyridin-2-yl)-1H-1,2,3-triazol-4-yl)phenyl)-6-azaspiro[2.5]octane (E14.02, 0.17 g).

Step 3. N-(4-(1-(6-(4,4-difluoropiperidin-1-yl)pyridin-2-yl)-1H-1,2,3-triazol-4-yl)-3-(6-azaspiro[2.5]octan-6-yl)phenyl)methanesulfonamide (Compound 29) was prepared from E14.02 and methansulfonamide in the manner described in Example 13, step 2.

Compounds in Table 6F were prepared from the indicated intermediate, amine, and sulfonamide in the same manner as Compound 29

TABLE 6F

| Name | Structure | Intermediate | Amine | Sulfonamide |
|---|---|---|---|---|
| Compound 61 | 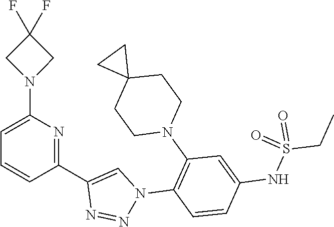 [SMILES: CCS(=O)(=O)Nc1ccc(-n2cc(-c3cccc(N4CC(F)(F)C4)n3)nn2)c(N2CCC3(CC2)CC3)c1] | T6X.03 | 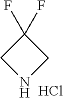 | 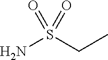 |
| Compound 70 | 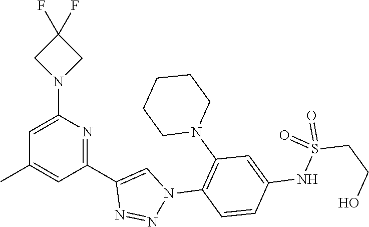 [SMILES: Cc1cc(-c2cn(-c3ccc(NS(=O)(=O)CCO)cc3N3CCCCC3)nn2)nc(N2CC(F)(F)C2)c1] | T6X.04 | 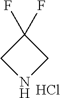 | 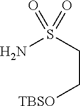 [SMILES: CC(C)(C)[Si](C)(C)OCCS(N)(=O)=O] |
| Compound 71 | 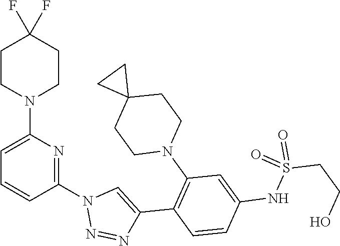 [SMILES: O=S(=O)(CCO)Nc1ccc(-c2cn(-c3cccc(N4CCC(F)(F)CC4)n3)nn2)c(N2CCC3(CC2)CC3)c1] | E14.01 | 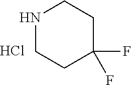 | 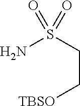 [SMILES: CC(C)(C)[Si](C)(C)OCCS(N)(=O)=O] |
| Compound 107 | 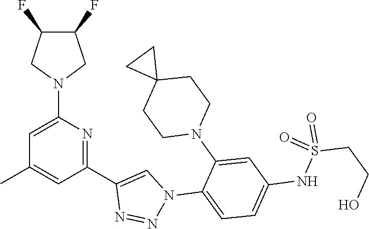 [SMILES: Cc1cc(-c2cn(-c3ccc(NS(=O)(=O)CCO)cc3N3CCC4(CC3)CC4)nn2)nc(N2C[C@@H](F)[C@@H](F)C2)c1] | T6X.07 | 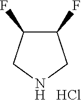 | 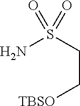 [SMILES: CC(C)(C)[Si](C)(C)OCCS(N)(=O)=O] |
| Compound 114 | 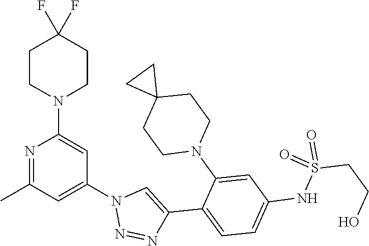 [SMILES: Cc1cc(-n2cc(-c3ccc(NS(=O)(=O)CCO)cc3N3CCC4(CC3)CC4)nn2)cc(N2CCC(F)(F)CC2)n1] | T6X.08 | 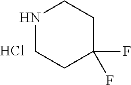 | 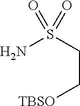 [SMILES: CC(C)(C)[Si](C)(C)OCCS(N)(=O)=O] |
| Compound 115 | 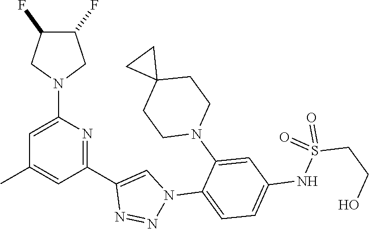 [SMILES: Cc1cc(-c2cn(-c3ccc(NS(=O)(=O)CCO)cc3N3CCC4(CC3)CC4)nn2)nc(N2C[C@@H](F)[C@H](F)C2)c1] | T6X.07 | 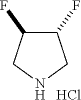 | 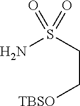 [SMILES: CC(C)(C)[Si](C)(C)OCCS(N)(=O)=O] |

### Example 1.5: Synthesis of N-(4-(4-(6-(4,4-difluoropiperidin-1-yl)pyridin-2-yl)-1H-1,2,3-triazol-1-yl)-3-(6-azaspiro[2.5]octan-6-yl)phenyl)-1-(hydroxymethyl)cyclopropane-1-sulfonamide (Compound 62)

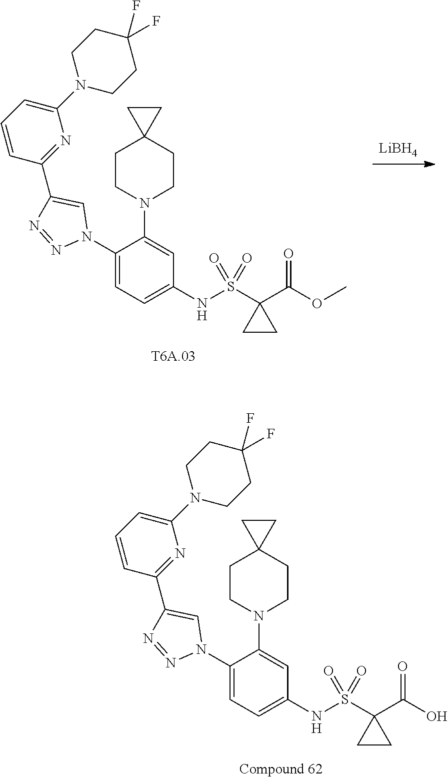 [SMILES: COC(=O)C1(S(=O)(=O)Nc2ccc(-n3cc(-c4cccc(N5CCC(F)(F)CC5)n4)nn3)c(N3CCC4(CC3)CC4)c2)CC1 ; "LiBH4" ; O=C(O)C1(S(=O)(=O)Nc2ccc(-n3cc(-c4cccc(N5CCC(F)(F)CC5)n4)nn3)c(N3CCC4(CC3)CC4)c2)CC1]

To a mixture of T6A.03 (50 mg, 80 μmol & 20 mg, 32 μmol) and THF (1 mL & 0.4 mL) was added LiBH₄ (10 mg, 0.48 mmol & 4 mg, 0.19 mmol) at −78° C. The mixtures were slowly warmed to 25° C. over 2 h. The mixtures were combined, poured into saturated aqueous NH₄Cl (20 mL), and extracted with EtOAc (2×10 mL). The combined extracts were washed with brine (10 mL), dried over Na₂SO₄, concentrated, and purified by preparative HPLC (C18; 45-75% MeCN in H₂O [formic acid]) to provide N-(4-(4-(6-(4,4-difluoropiperidin-1-yl)pyridin-2-yl)-1H-1,2,3-triazol-1-yl)-3-(6-azaspiro[2.5]octan-6-yl)phenyl)-1-(hydroxymethyl)cyclopropane-1-sulfonamide (Compound 62; 8 mg).

Compounds in Table 6H were prepared from the indicated esters in the same manner as Compound 62.

TABLE 6H

| Name | Structure | Ester |
|---|---|---|
| Compound 80 | 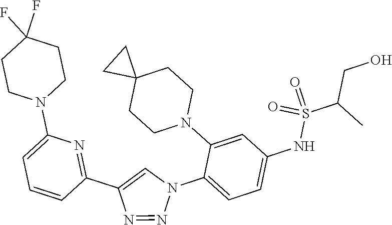 [SMILES: CC(CO)S(=O)(=O)Nc1ccc(-n2cc(-c3cccc(N4CCC(F)(F)CC4)n3)nn2)c(N2CCC3(CC2)CC3)c1] | T6A.04 |
| Compound 91 | 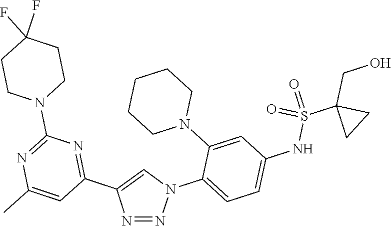 [SMILES: Cc1cc(-c2cn(-c3ccc(NS(=O)(=O)C4(CO)CC4)cc3N3CCCCC3)nn2)nc(N2CCC(F)(F)CC2)n1] | T6A.05 |
| Compound 122 | 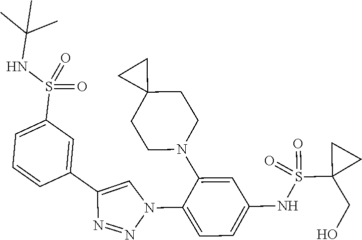 [SMILES: CC(C)(C)NS(=O)(=O)c1cccc(-c2cn(-c3ccc(NS(=O)(=O)C4(CO)CC4)cc3N3CCC4(CC3)CC4)nn2)c1] | E15.01 |
| Compound 157 | 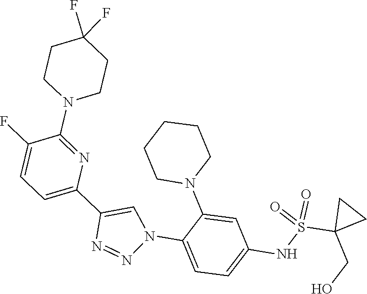 [SMILES: O=S(=O)(Nc1ccc(-n2cc(-c3ccc(F)c(N4CCC(F)(F)CC4)n3)nn2)c(N2CCCCC2)c1)C1(CO)CC1] | T6A.08 |
| Compound 160 | 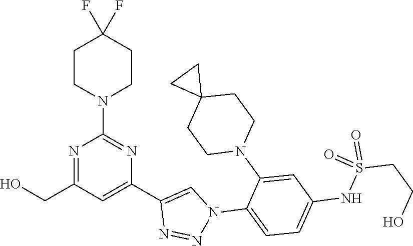 [SMILES: O=S(=O)(CCO)Nc1ccc(-n2cc(-c3cc(CO)nc(N4CCC(F)(F)CC4)n3)nn2)c(N2CCC3(CC2)CC3)c1] | E15.02 |

### Example 15A: Synthesis of N-(4-(4-(6-(cyclopentyloxy)pyridin-2-yl)-1H-1,2,3-triazol-1-yl)-3-(6-azaspiro[2.5]octan-6-yl)phenyl)-2-hydroxyethane-1-sulfonamide (Compound 100)

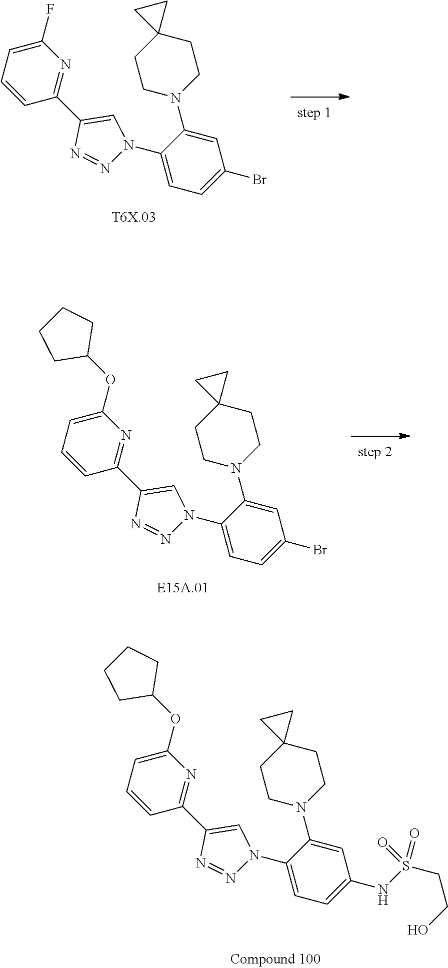 [SMILES: Fc1cccc(-c2cn(-c3ccc(Br)cc3N3CCC4(CC3)CC4)nn2)n1 ; Brc1ccc(-n2cc(-c3cccc(OC4CCCC4)n3)nn2)c(N2CCC3(CC2)CC3)c1 ; O=S(=O)(CCO)Nc1ccc(-n2cc(-c3cccc(OC4CCCC4)n3)nn2)c(N2CCC3(CC2)CC3)c1]

Step 1. To a mixture of T6X.03 (0.50 g, 1.2 mmol, 1.00 eq), cyclopentanol (0.15 g, 1.8 mmol), and THF (10 mL) was added KOtBu (0.46 g, 4.1 mmol). The mixture was stirred at 80° C. for 3 h, poured into H₂O (50 mL), and extracted with EtOAc (2×50 mL). The extracts were combined, washed with brine (30 mL), dried over Na₂SO₄, concentrated, and purified by silica chromatography (0-100% EtOAc in PE) to provide 6-[5-bromo-2-[4-[6-(cyclopentoxy)-2-pyridyl]triazol-1-yl]phenyl]-6-azaspiro[2.5]octane (E15A.01, 0.40 g).

Step 2. A degassed mixture of E15A.01 (0.20 g, 0.40 mmol), 2-[tert-butyl(dimethyl)silyl]oxyethanesulfonamide (0.12 g, 0.49 mmol), Pd(dba)₂ (5 mg, 8 μmol), t-Bu Xphos (7 mg, 16 μmol), K₂CO₃ (0.11 g, 0.81 mmol), and 2-MeTHF (5 mL) was stirred at 100° C. for 12 h under an N₂ atmosphere. The reaction was poured into H₂O (30 mL) and extracted with EtOAc (2×30 mL). The combined extracts were washed with brine (10 mL), dried over Na₂SO₄, filtered, and concentrated. The residue was stirred in 5 mL of EtOAc and 5 mL of 2 M HCl for 2 h. H₂O (30 mL) was added, and the mixture was extracted with EtOAc (2×30 mL). The combined extracts were washed with brine (10 mL), dried over Na₂SO₄, filtered, concentrated, and purified by preparative HPLC (C18; 50-85% MeCN in H₂O [0.1% formic acid]) to provide N-(4-(4-(6-(cyclopentyloxy)pyridin-2-yl)-1H-1,2,3-triazol-1-yl)-3-(6-azaspiro[2.5]octan-6-yl)phenyl)-2-hydroxyethane-1-sulfonamide (Compound 100. 36 mg).

### Synthesis of N-(4-(1-(6-(cyclopentyloxy)pyridin-2-yl)-1H-1,2,3-triazol-4-yl)-3-(6-azaspiro[2.5]octan-6-yl)phenyl)-2-hydroxyethane-1-sulfonamide (Compound 113)

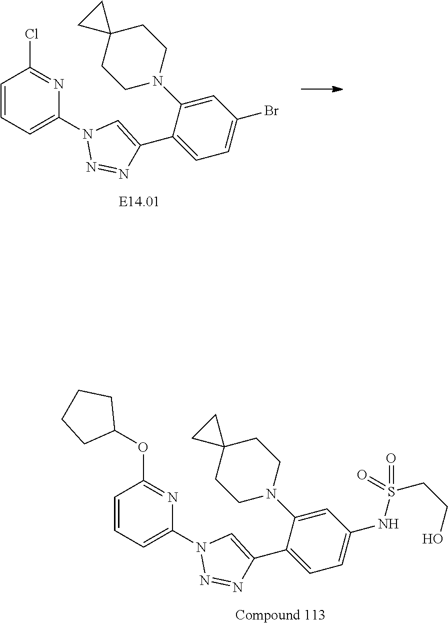 [SMILES: Clc1cccc(-n2cc(-c3ccc(Br)cc3N3CCC4(CC3)CC4)nn2)n1 ; O=S(=O)(CCO)Nc1ccc(-c2cn(-c3cccc(OC4CCCC4)n3)nn2)c(N2CCC3(CC2)CC3)c1]

Compound 113 was prepared in two steps from E14.01 in the manner described for Compound 100.

### Synthesis of methyl 1-(N-(4-(4-(3-(N-(tert-butyl)sulfamoyl)phenyl)-1H-1,2,3-triazol-1-yl)-3-(6-azaspiro[2.5]octan-6-yl)phenyl)sulfamoyl)cyclopropane-1-carboxylate (E15.01)

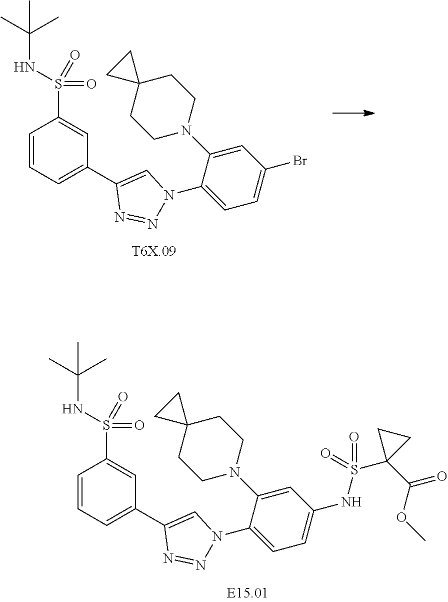 [SMILES: CC(C)(C)NS(=O)(=O)c1cccc(-c2cn(-c3ccc(Br)cc3N3CCC4(CC3)CC4)nn2)c1 ; COC(=O)C1(S(=O)(=O)Nc2ccc(-n3cc(-c4cccc(S(=O)(=O)NC(C)(C)C)c4)nn3)c(N3CCC4(CC3)CC4)c2)CC1]

A degassed mixture of T6X.09 (0.20 g, 0.37 mmol), methyl 1-sulfamoylcyclopropane-1-carboxylate (80 mg, 0.45 mmol), Pd₂(dba)₃ (17 mg, 19 μmol), t-Bu Xphos (13 mg, 30 μmol), and K₂CO₃ (0.10 g, 0.74 mmol), and 2-MeTHF (4 mL) was stirred at 100° C. for 3 h under an N₂ atmosphere. The mixture was combined with H₂O (30 mL), extracted with EtOAc (2×30 mL), and the combined extracts were washed with brine (10 mL), dried over Na₂SO₄, concentrated, and purified by silica chromatography (0-45% EtOAc in PE) to provide methyl 1-(N-(4-(4-(3-(N-(tert-butyl)sulfamoyl)phenyl)-1H-1,2,3-triazol-1-yl)-3-(6-azaspiro[2.5]octan-6-yl)phenyl)sulfamoyl)cyclopropane-1-carboxylate (E15.01, 0.25 g).

Compounds in the following table were prepared from the indicated aryl halide via that procedure described for the synthesis of E15.01.

| Compound | Structure | Halide | Sulfon-amide |
|---|---|---|---|
| Compound 130 | 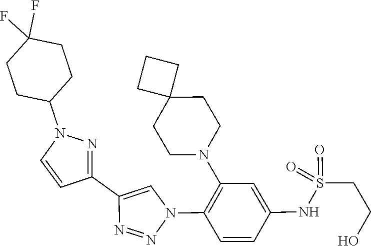 [SMILES: O=S(=O)(CCO)Nc1ccc(-n2cc(-c3ccn(C4CCC(F)(F)CC4)n3)nn2)c(N2CCC3(CCC3)CC2)c1] | T6X.10 | 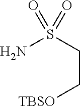 [SMILES: CC(C)(C)[Si](C)(C)OCCS(N)(=O)=O] |
| Compound 154 | 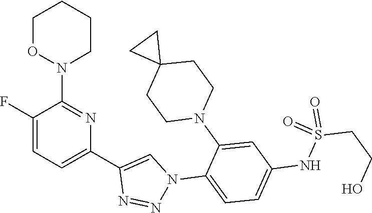 [SMILES: O=S(=O)(CCO)Nc1ccc(-n2cc(-c3ccc(F)c(N4CCCCO4)n3)nn2)c(N2CCC3(CC2)CC3)c1] | T6X.12 | 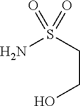 |
| Compound 155 | 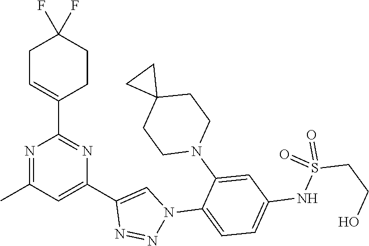 [SMILES: Cc1cc(-c2cn(-c3ccc(NS(=O)(=O)CCO)cc3N3CCC4(CC3)CC4)nn2)nc(C2=CCC(F)(F)CC2)n1] | T6X.13 | 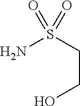 |
| E15.02 | 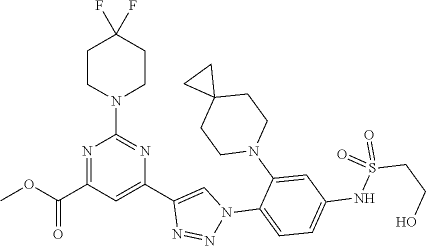 [SMILES: COC(=O)c1cc(-c2cn(-c3ccc(NS(=O)(=O)CCO)cc3N3CCC4(CC3)CC4)nn2)nc(N2CCC(F)(F)CC2)n1] | T6X.16 | 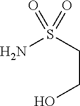 |
| Compound 159 | 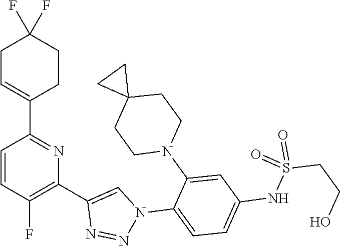 [SMILES: O=S(=O)(CCO)Nc1ccc(-n2cc(-c3nc(C4=CCC(F)(F)CC4)ccc3F)nn2)c(N2CCC3(CC2)CC3)c1] | T6X.15 | 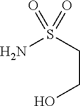 |
| Compound 162 | 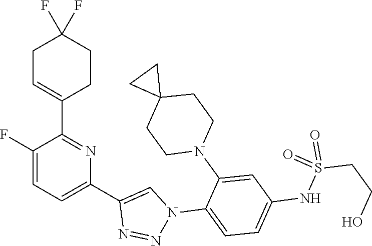 [SMILES: O=S(=O)(CCO)Nc1ccc(-n2cc(-c3ccc(F)c(C4=CCC(F)(F)CC4)n3)nn2)c(N2CCC3(CC2)CC3)c1] | T6X.14 | 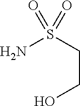 |

### Example 15B. Synthesis of N-(4-(4-(5-cyclopentylfuran-2-yl)-1H-1,2,3-triazol-1-yl)-3-(6-azaspiro[2.5]octan-6-yl)phenyl)-2-hydroxyethane-1-sulfonamide (Compound 126)

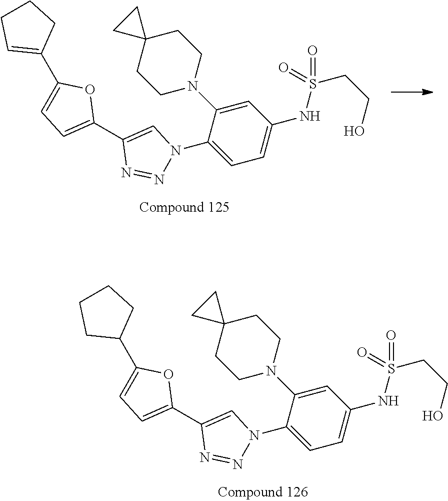 [SMILES: O=S(=O)(CCO)Nc1ccc(-n2cc(-c3ccc(C4=CCCC4)o3)nn2)c(N2CCC3(CC2)CC3)c1 ; O=S(=O)(CCO)Nc1ccc(-n2cc(-c3ccc(C4CCCC4)o3)nn2)c(N2CCC3(CC2)CC3)c1]

A N₂ purged mixture of Compound 125 (20 mg, 39 μmol), MeOH (2 mL), 10% Pd/C (3 mg) was stirred under H₂ (15 Psi) at 25° C. for 2 h. The mixture was filtered, concentrated, and purified by preparative HPLC (C18; 55-85% MeCN in H₂O [HCl modifier]) to provide N-(4-(4-(5-cyclopentylfuran-2-yl)-1H-1,2,3-triazol-1-yl)-3-(6-azaspiro[2.5]octan-6-yl)phenyl)-2-hydroxyethane-1-sulfonamide (Compound 126, 11 mg).

### Example 15C. Synthesis of N-(4-(4-(6-(4,4-difluoropiperidin-1-yl)-4-(hydroxymethyl)pyridin-2-yl)-1H-1,2,3-triazol-1-yl)-3-(6-azaspiro[2.5]octan-6-yl)phenyl)ethanesulfonamide (Compound 131)

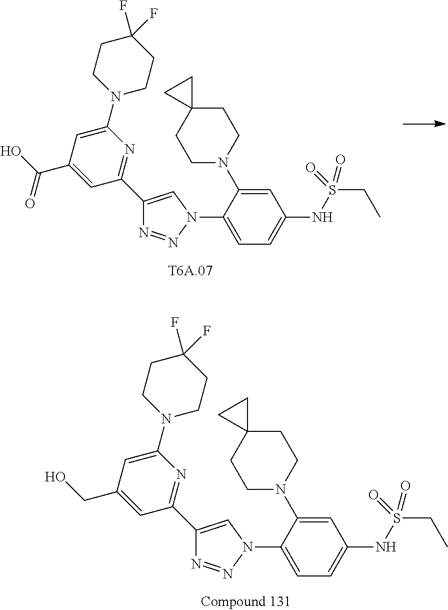 [SMILES: CCS(=O)(=O)Nc1ccc(-n2cc(-c3cc(C(=O)O)cc(N4CCC(F)(F)CC4)n3)nn2)c(N2CCC3(CC2)CC3)c1 ; CCS(=O)(=O)Nc1ccc(-n2cc(-c3cc(CO)cc(N4CCC(F)(F)CC4)n3)nn2)c(N2CCC3(CC2)CC3)c1]

To a mixture of T6A.07 (34 mg, 57 μmol) and THF (2 mL) was added BH₃-Me₂S (10 M, 0.28 mL, 35 mmol) at 0° C. The mixture was stirred at 80° C. for 2 h, and 2N HCl (1 mL) was added slowly at 5-10° C. Then H₂O (10 mL) was added and the mixture was extracted with EtOAc (20 mL×2). The combined extracts were washed with brine (20 mL), dried over Na₂SO₄, filtered, concentrated, and purified by preparative HPLC (40-70% MeCN in H₂O [HCl modifier]) to provide N-(4-(4-(6-(4,4-difluoropiperidin-1-yl)-4-(hydroxymethyl)pyridin-2-yl)-1H-1,2,3-triazol-1-yl)-3-(6-azaspiro[2.5]octan-6-yl)phenyl)ethanesulfonamide (Compound 131, 6 mg).

### Example 15D. Synthesis of 3-(4,4-difluoropiperidin-1-yl)-5-(1-(4-(ethylsulfonamido)-2-(6-azaspiro[2.5]octan-6-yl)phenyl)-1H-1,2,3-triazol-4-yl)pyrazine 1-oxide (Compound 146)

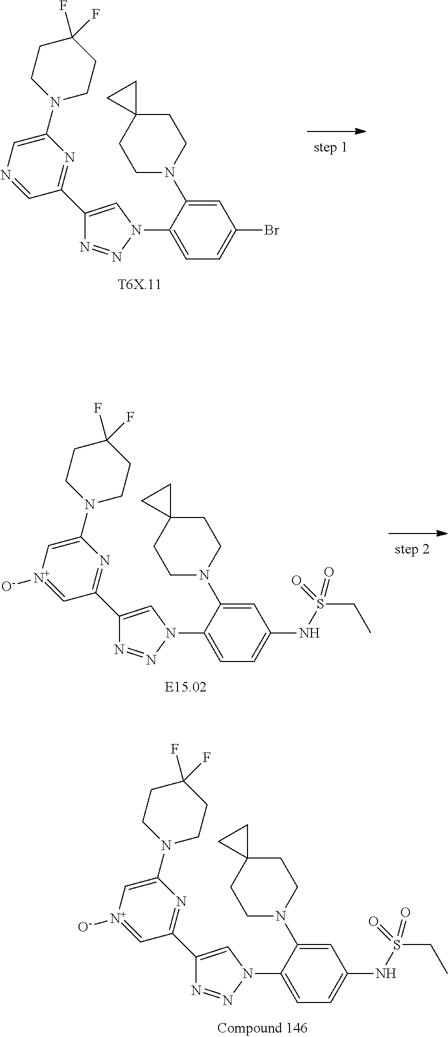 [SMILES: FC1(F)CCN(c2cncc(-c3cn(-c4ccc(Br)cc4N4CCC5(CC4)CC5)nn3)n2)CC1 ; CCS(=O)(=O)Nc1ccc(-n2cc(-c3c[n+]([O-])cc(N4CCC(F)(F)CC4)n3)nn2)c(N2CCC3(CC2)CC3)c1 ; CCS(=O)(=O)Nc1ccc(-n2cc(-c3c[n+]([O-])cc(N4CCC(F)(F)CC4)n3)nn2)c(N2CCC3(CC2)CC3)c1]

Step 1. To a 0° C. mixture of T6X.11 (0.20 g, 0.38 mmol) and CHCl₃ (30 mL) was added m-CPBA (0.11 g, 0.57 mmol, 85% purity). The mixture was stirred at 20° C. for 3 h, poured into 1M Na₂SO₃ (10. mL) and the resulting mixture was extracted with CH₂Cl₂ (2×10 mL). The extracts were combined, washed with brine (10 mL), dried over Na₂SO₄, filtered, concentrated, and purified by silica chromatography (0-50% EtOAc in PE) to provide 6-[5-bromo-2-[4-[6-(4,4-difluoro-1-piperidyl)-4-oxido-pyrazin-4-ium-2-yl]triazol-1-yl]phenyl]-6-azaspiro[2.5]octane (E15.02, 0.17 g)

Step 2. 3-(4,4-difluoropiperidin-1-yl)-5-(1-(4-(ethylsulfonamido)-2-(6-azaspiro[2.5]octan-6-yl)phenyl)-1H-1,2,3-triazol-4-yl)pyrazine 1-oxide (Compound 146) was prepared from E15.02 and ethanesulfonamide in the manner described in Example 13, step 2.

### Example 16: Synthesis of N-(4-(3-(6-morpholinopyridin-2-yl)-1,2,4-oxadiazol-5-yl)-3-(6-azaspiro[2.5]octan-6-yl)phenyl)methanesulfonamide (Compound 163)

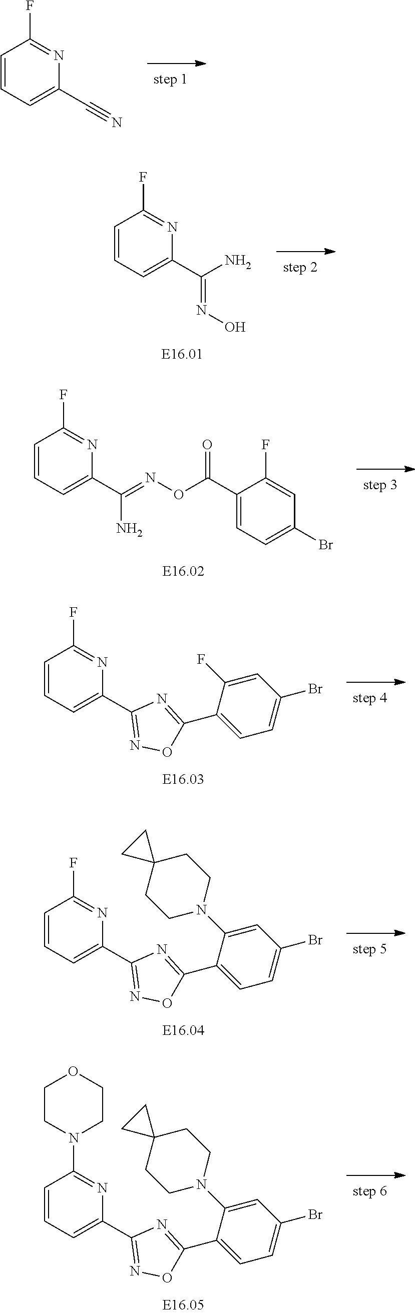 [SMILES: N#Cc1cccc(F)n1 ; N/C(=N\O)c1cccc(F)n1 ; N/C(=N\OC(=O)c1ccc(Br)cc1F)c1cccc(F)n1 ; Fc1cccc(-c2noc(-c3ccc(Br)cc3F)n2)n1 ; Fc1cccc(-c2noc(-c3ccc(Br)cc3N3CCC4(CC3)CC4)n2)n1 ; Brc1ccc(-c2nc(-c3cccc(N4CCOCC4)n3)no2)c(N2CCC3(CC2)CC3)c1]

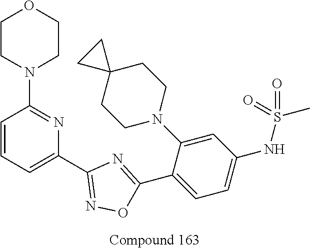 [SMILES: CS(=O)(=O)Nc1ccc(-c2nc(-c3cccc(N4CCOCC4)n3)no2)c(N2CCC3(CC2)CC3)c1]

Step 1. To a mixture of 6-fluoropyridine-2-carbonitrile (5.0 g, 41 mmol), and EtOH (4 mL) was added iPr₂NEt (21 mL, 123 mmol) and NH₂OH hydrochloride (5.7 g, 82 mmol). The mixture was stirred at 25° C. for 12 h, then was combined with H₂O (0.5 L). The mixture was extracted with EtOAc (0.4 L x 3). The combined extracts were washed with brine (0.4 L), dried over Na₂SO₄, filtered, and concentrated to provide 6-fluoro-N′-hydroxy-pyridine-2-carboxamidine (E16.01, 9.8 g).

Step 2. To a mixture of E16.01 (1.0 g, 6.5 mmol), CH₂Cl₂ (30 mL), iPr₂NEt (2.3 mL, 13 mmol) at 0° C., was added 4-bromo-2-fluoro-benzoyl chloride (1.8 g, 7.7 mmol) was added dropwise. The resulting mixture was stirred at 0° C. for 12 h, then was concentrated to provide [(E)-[amino-(6-fluoro-2-pyridyl)methylene]amino] 4-bromo-2-fluoro-benzoate (E16.02, 5.8 g, crude).

Step 3. A mixture of E16.02 (5.80 g, 16 mmol), toluene (50 mL), and K₂CO₃ (6.8 g, 49 mmol) was stirred at 110° C. for 12 h. The mixture was cooled, combined with CH₂Cl₂ (100 mL×2). The combined extracts were washed with saturated NaHCO₃ (200 mL), concentrated, and purified by silica chromatography (0-100% EtOAc in PE) to provide 5-(4-bromo-2-fluoro-phenyl)-3-(6-fluoro-2-pyridyl)-1,2,4-oxadiazole (E16.03, 1.4 g).

Step 4. A mixture of E16.03 (1.1 g, 3.3 mmol), DMF (25 mL), K₂CO₃ (1.4 g, 9.8 mmol), and 6-azaspiro[2.5]octane (0.54 g, 4.9 mmol) was stirred at 60° C. for 12 h. The mixture was concentrated and purified by silica chromatography (0-100% EtOAc in PE) to provide 5-[2-(6-azaspiro[2.5]octan-6-yl)-4-bromo-phenyl]-3-(6-fluoro-2-pyridyl)-1,2,4-oxadiazole (E16.04, 0.35 mg).

Step 5. A mixture of E16.04 (0.27 g, 0.63 mmol), DMF (13 mL), K₂CO₃ (0.26 g, 1.9 mmol), morpholine (82 mg, 0.94 mmol). The mixture was stirred at 110° C. for 24 h, concentrated, and purified by silica chromatography (0-100% EtOAc in PE) to provide 4-[6-[5-[2-(6-azaspiro[2.5]octan-6-yl)-4-bromo-phenyl]-1,2,4-oxadiazol-3-yl-1]-2-pyridyl]morpholine (E16.05, 0.24 g).

Step 6. A degassed mixture of E16.04 (0.24 g, 0.49 mmol), methanesulfonamide (0.14 g, 1.5 mmol), N¹,N²-dimethylcyclohexane-1,2-diamine (70 mg, 0.49 mmol), DMA (20 mL), Bis[(tetrabutylammonium iodide)copper(I) iodide] (0.55 g, 0.49 mmol), and Cs₂CO₃ (0.40 g, 1.2 mmol), was stirred at 100° C. for 2 h under an N₂ atmosphere. The mixture was diluted with 10 mL of H₂O and extracted with EtOAc (10 mL×3). The combined extracts were washed with brine (30 mL), dried over Na₂SO₄, filtered, concentrated, and purified by preparative HPLC (C18, 40-70% MeCN in H₂O [0.1% formic acid]) to provide N-(4-(3-(6-morpholinopyridin-2-yl)-1,2,4-oxadiazol-5-yl)-3-(6-azaspiro[2.5]octan-6-yl)phenyl)methanesulfonamide (Compound 163, 46 mg).

### Example 17. Synthesis of N-(4-(5-(6-morpholinopyridin-2-yl)-1,2,4-oxadiazol-3-yl)-3-(6-azaspiro[2.5]octan-6-yl)phenyl)methanesulfonamide (Compound 164)

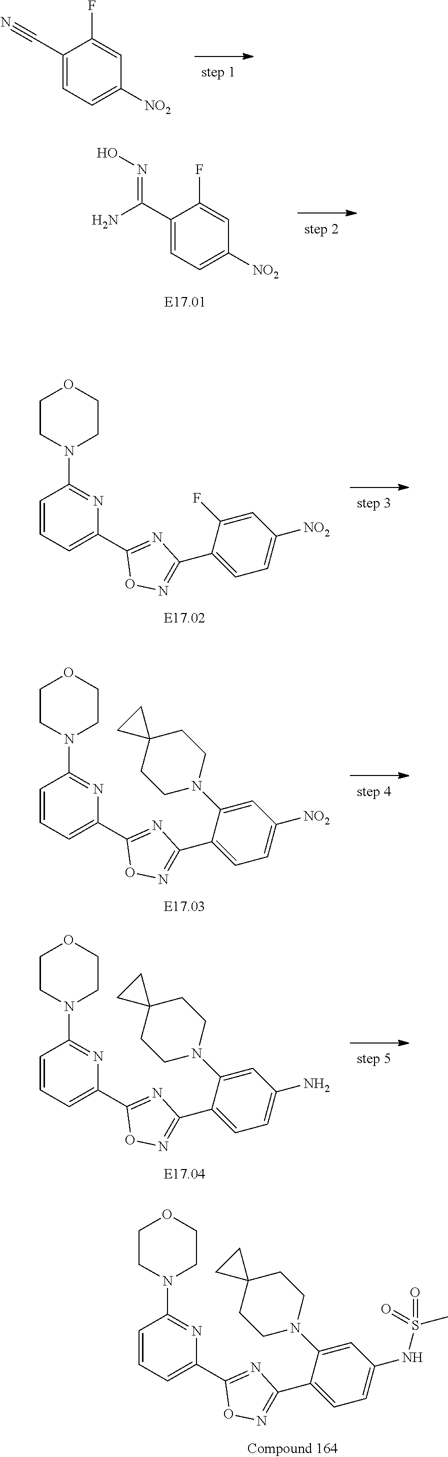 [SMILES: N#Cc1ccc([N+](=O)[O-])cc1F ; N/C(=N\O)c1ccc([N+](=O)[O-])cc1F ; O=[N+]([O-])c1ccc(-c2noc(-c3cccc(N4CCOCC4)n3)n2)c(F)c1 ; O=[N+]([O-])c1ccc(-c2noc(-c3cccc(N4CCOCC4)n3)n2)c(N2CCC3(CC2)CC3)c1 ; Nc1ccc(-c2noc(-c3cccc(N4CCOCC4)n3)n2)c(N2CCC3(CC2)CC3)c1 ; CS(=O)(=O)Nc1ccc(-c2noc(-c3cccc(N4CCOCC4)n3)n2)c(N2CCC3(CC2)CC3)c1]

Step 1. A mixture of 2-fluoro-4-nitro-benzonitrile (2.0 g, 12 mmol) and NH₂OH hydrochloride (2.6 g, 37 mmol), EtOH (18 mL), and NaHCO₃ (3.2 g, 38 mmol) in 1.5 mL of H₂O. The mixture was stirred at 85° C. for 6 h and concentrated to remove EtOH. The residue was dissolved with EtOAc (30 mL) and the solution was washed water (15 mL) dried over Na₂SO₄, filtered, concentrated, and triturated with iPrOH (10 mL) at 0° C. for 20 min. The suspension was filtered, the filter cake was washed with cooled iPrOH (1 mL×3) and dried under reduced pressure to give 2-fluoro-N′-hydroxy-4-nitrobenzimidamide (E17.01, 1.9 g).

Step 2. A mixture of E17.01 (1.3 g, 6.5 mmol), THF (60 mL), and NaOEt (2.0 g, 29 mmol) was stirred at 20° C. for 15 min. 6-Morpholinopicolinoyl chloride hydrochloride (2.0 g, 7.6 mmol) was added in portions. The mixture was stirred at 80° C. for 12 h, diluted with THF (80 mL) and EtOAc (50 mL), and 2M HCl was added until the pH was neutral. The organic phase was washed with brine (50 mL×2), dried over Na₂SO₄, filtered, concentrated, and silica chromatography (0-80% [1:1 EtOAc/THF] in PE) to provide 4-[6-[3-(2-fluoro-4-nitro-phenyl)-1,2,4-oxadiazol-5-yl]-2-pyridyl]morpholine (E17.02, 1 g).

Step 3. A mixture of E17.02 (0.60 g, 1.6 mmol) and 6-azaspiro[2.5]octane hydrochloride (0.60 g, 4.1 mmol), and NMP (12 mL), and K₂CO₃ (1.1 g, 8.1 mmol) was stirred at 120° C. for 12 h. H₂O (36 mL) was added dropwise, and the resulting mixture was filtered and washed with water (5 mL×3). The filter cake was dissolved in EtOAc (40 mL), washed with brine (10 mL), concentrated, and triturated with MTBE (5 mL) at 20° C. for 15 min. The suspension was filtered and the filter-cake was washed with MTBE (3 mL×2) and dried to provide 4-(6-(3-(4-nitro-2-(6-azaspiro[2.5]octan-6-yl)phenyl)-1,2,4-oxadiazol-5-yl)pyridin-2-yl)morpholine (E17.03, 0.31 g).

Step 4. A mixture of E17.03 (0.30 g, 0.65 mmol), SnCl₂ dihydrate (1.7 g, 7.5 mmol), THF (7 mL), and EtOH (20 mL) was stirred at 100° C. for 12 h. Saturated NaHCO₃(60 mL) was added, and the resulting suspension was filtered through celite. The filtrate was extracted with 50 mL of EtOAc (50 mL) and the extract was concentrated and purified by silica chromatography (0-40% MeOH in CH₂Cl₂) to provide 4-(5-(6-morpholinopyridin-2-yl)-1,2,4-oxadiazol-3-yl)-3-(6-azaspiro[2.5]octan-6-yl)aniline (E17.04, 80 mg).

Step 5. To a mixture of E17.04 (60 mg, 0.14 mmol), CH₂Cl₂ (3 mL) and Et₃N (42 mg, 0.42 mol) was added MsCl (50 mg, 0.44 mmol) and the mixture was stirred at 20° C. for 1 h. H₂O (5 mL) was added, and the mixture was extracted with CH₂Cl₂ (10 mL). The extract was washed with brine (5 mL), filtered, concentrated, and purified by preparative TLC (SiO₂, 8:1 CH₂Cl₂/MeOH) to provide N-(4-(5-(6-morpholinopyridin-2-yl)-1,2,4-oxadiazol-3-yl)-3-(6-azaspiro[2.5]octan-6-yl)phenyl)methanesulfonamide (Compound 164, 2.1 mg).

Table 6G describes the chromatography separation of isomers for specific examples.

TABLE 6G

| Compound | Conditions | First | Purity % | Second | Purity % |
|---|---|---|---|---|---|
| 51 | Chiralcel OX | 51a | 100% | 51b | 100% |
| | (250 mm × 30 mm, 10 μm) | | (er) | | (er) |
| | 50% [iPrOH] in supercritical CO₂ | | | | |
| 90 | Chiralpak IG | 90a | 100% | 90b | 99.1% |
| | (250 mm × 30 mm, 10 μm) | | (er) | | (er) |
| | 20-45% [EtOH (0.1% NH₃)] in | | | | |
| | supercritical CO₂ | | | | |

TABLE 7
Spectroscopic Data

| | ESI MS | |
|---|---|---|
| Compound | (m/z) | NMR summary (400 MHz) ppm |
| Compound | 497.0, | (DMSO-d⁶) δ 7.77-7.64 (m, 2H), 7.42 (br d, J = 7.2 Hz, 1H), 7.22-7.19 |
| 1 | 495.1 | (m, 2H), 6.92 (br d, J = 8.8 Hz, 1H), 3.77-3.70 (m, 4H), 3.61 (m, 4H), |
| | (M + H) | 2.92 (m, 4H), 1.50-1.35 (m, 4H), 0.28 (s, 4H) |
| Compound | 510.3 | (DMSO-d⁶) δ 9.89 (br s, 1H), 7.79-7.61 (br s, 2H), 7.42 (br s, 1H), |
| 2 | (M + H)⁺ | 7.07-6.83 (m, 3H), 3.73 (m, 4H), 3.59 (m, 4H), 3.04 (br s, 3H), 2.87 |
| | | (m, 4H), 1.44 (m, 4H), 0.36-0.23 (m, 4H) |
| Compound | 480.3 | (80° C., DMSO-d⁶) δ 13.4 (br s, 1H), 9.55 (br s, 1H), 8.10-7.79 (m, |
| 3 | (M + H)⁺ | 1H), 7.68 (br d, J = 6.0 Hz, 1H), 7.24-6.89 (m, 4H), 4.49-4.29 (m, 2H), |
| | | 3.02-2.90 (m, 5H), 2.72-2.80 (m, 2H), 2.42 (s, 1H), 2.11-1.98 (m, 2H), |
| | | 1.62-1.56 (m, 1H), 1, 50-1.40 (m, 3H), 1.32-1.24 (m, 1H), 0.40-0.23 |
| | | (m, 4H) |
| Compound | 418.1 | (DMSO-d⁶) δ 8.25 (br d, J = 3.2 Hz, 1H) 7.94 (br d, J = 6.0 Hz, 1H) |
| 4 | (M + H)⁺ | 7.77-7.66 (m, 1H) 7.41 (d, J = 7.6 Hz, 1H) 6.99-6.87 (m, 2H) 3.82- |
| | | 3.68 (m, 4H) 3.64-3.54 (m, 4H) 3.16 (m, 4H) 1.37 (m, 4H) 0.27 (br s, |
| | | 4H) |
| Compound | 542.3 | (DMSO-d⁶) δ 14.10 (br s, 1 H) 9.64 (br s, 1 H) 7.61-7.92 (m, 2 H) |
| 5 | (M + H)⁺ | 7.34-7.59 (m, 1 H) 6.84-7.29 (m, 3 H) 3.67-4.00 (m, 2 H) 3.01 (br |
| | | s, 3 H) 2.82-2.99 (m, 4 H) 1.89-2.25 (m, 4 H) 1.29-1.69 (m, 4 H) |
| | | 0.31 (br s, 4 H) |
| Compound | 525.1 | (DMSO-d⁶) δ 7.75-7.62 (m, 1H), 7.32-7.16 (m, 1H), 7.11-7.01 (m, |
| 6 | (M + H)⁺ | 1H), 7.00-6.87 (m, 1H), 3.88-3.77 (m, 4H), 3.69 (br d, J = 3.9 Hz, 4H), |
| | | 3.05 (s, 3H), 2.92-2.77 (m, 4H), 2.38 (s, 3H), 1.58-1.37 (m, 4H), 0.29 |
| | | (s, 4H) |
| Compound | 616.1 | (DMSO-d⁶) δ 7.63-7.78 (m, 2 H) 7.38-7.57 (m, 1 H) 6.91-7.24 (m, |
| 7 | (M + H)⁺ | 3 H) 4.07-4.26 (m, 4 H) 3.85 (br s, 4 H) 2.91 (br s, 4 H) 1.95-2.16 |
| | | (m, 4 H) 1.39-1.62 (m, 4 H) 1.15-1.29 (m, 3 H) 0.23-0.43 (m, 4 H) |
| Compound | 574.1 | (DMSO-d⁶) δ 9.82 (br s, 1 H) 7.71-7.67 (m, 2 H) 7.41 (s, 1 H) 7.05- |
| 8 | (M + H)⁺ | 6.91 (m, 3 H) 4.90(br s, 1 H) 3.85-3.74 (m, 6 H) 3.29-3.28 (m, 2 H) |
| | | 2.89-2.83 (m, 4 H) 2.03-2.01 (m, 4 H) 1.44-1.23 (m, 4 H) 0.31- |
| | | 0.24 (m, 4 H) |
| Compound | 543.2 | (DMSO-d⁶) δ 12.22-11.86 (m, 1 H), 8.13-7.83 (m, 1 H), 7.74-7.53 |
| 9 | (M + H)⁺ | (m, 2 H), 7.37-7.10 (m, 2 H), 7.09-7.00 (m, 1 H), 6.85-6.67 (m, 1 |
| | | H), 3.86-3.73 (m, 4 H), 3.03 (s, 3 H), 2.93 (br s4 H), 2.08 (s, 4 H), |
| | | 1.59 (s, 4 H), 0.38 (s, 4 H |
| Compound | 509.1 | (DMSO-d⁶) δ 12.15-11.97 (m, 1 H) 9.866 (s, 1 H) 8.08-7.81 (m, 1 |
| 10 | (M + H)⁺ | H) 7.70-7.65 (m, 1 H) 7.62-7.54 (m, 1 H) 7.31-7.07 (m, 2 H) 7.06- |
| | | 6.96 (m, 1 H) 6.72-6.63 (m, 1 H) 3.80-3.70 (m, 4 H) 3.62-3.46 (m, |
| | | 4 H) 3.04 (s, 3 H) 2.93-2.79 (m, 4 H) 1.72-1.45 (m, 4 H) 0.39-0.31 |
| | | (m, 4 H) |
| Compound | 544.2 | (DMSO-d⁶) δ 10.08 (s, 1 H) 9.07 (s, 1 H) 7.71 (dd, J = 8.4, 7.4 Hz, 1 |
| 11 | (M + H)⁺ | H) 7.58-7.60 (d, J = 8.6 Hz, 1 H) 7.36-7.38 (d, J = 7.2 Hz, 1 H) 7.12 |
| | | (d, J = 2.2 Hz, 1 H) 7.05 (dd, J = 8.8, 2.20 Hz, 1 H) 6.96-6.98 (d, J = |
| | | 8.8 Hz, 1 H) 3.78-3.81 (m, 4 H) 3.10 (s, 3 H) 2.67-2.69 (m, 4 H) |
| | | 1.93-2.09 (m, 4 H) 1.32 (m, 4 H) 0.25 (s, 4 H) |
| Compound | 579.1 | (DMSO-d⁶) δ 10.23-9.95 (m, 1H), 9.36-9.24 (m, 1H), 8.44-8.42 |
| 13 | (M + H)⁺ | (m, 1H), 8.40-8.37 (m, 1H), 7.93-7.81 (m, 2H), 7.55-7.47 (m, 1H), |
| | | 7.16-7.05 (m, 1H), 7.05-6.96 (m, 1H), 4.39-4.28 (m, 4H), 3.13- |
| | | 3.06 (m, 3H), 2.76-2.61 (m, 4H), 1.26 (br d, J = 3.9 Hz, 4H), 0.18 (br |
| | | s, 4H) |
| Compound | 558.1 | (DMSO-d⁶) δ 10.05 (s, 1 H) 9.02 (s, 1 H) 7.59 (d, J = 8.53 Hz, 1 H) |
| 14 | (M + H)⁺ | 7.25 (s, 1 H) 7.11 (d, J = 2.26 Hz, 1 H) 7.03 (dd, J = 8.60, 2.20 Hz, 1 H) |
| | | 6.81 (s, 1 H) 3.79 (br t, J = 5.33 Hz, 4 H) 3.09 (s, 3 H) 2.68 (br t, J = 4.96 |
| | | Hz, 4 H) 2.33 (s, 3 H) 2.09-1.89 (m, 4 H) 1.31 (br s, 4 H) 0.24 (s, 4 |
| | | H) |
| Compound | 518.1 | (DMSO-d⁶) δ 9.99 (s, 1 H), 8.97 (s, 1 H), 7.69 (t, J = 8.22 Hz, 1 H), |
| 15 | (M + H)⁺ | 7.55 (d, J = 8.33 Hz, 1 H), 7.37 (d, J = 7.45 Hz, 1 H), 7.06 (d, J = 1.97 Hz, |
| | | 1 H), 7.01 (dd, J = 8.66, 1.86 Hz, 1 H), 6.95 (d, J = 8.55 Hz, 1 H), 3.79 |
| | | (br d, J = 5.92 Hz, 4 H), 3.07 (s, 3 H), 2.64-2.59 (m, 4 H), 2.02-1.92 |
| | | (m, 4 H), 1.46 (br s, 6 H) |
| Compound | 543.1 | (DMSO-d⁶) δ 9.91 (s, 1H), 8.93 (s, 1H), 8.20 (s, 1H), 7.60 (t, J = 7.9 |
| 17 | (M + H)⁺ | Hz, 1H), 7.51 (d, J = 8.6 Hz, 1H), 7.10-7.03 (m, 2H), 7.00-6.96 (m, |
| | | 1H), 6.82 (d, J = 8.6 Hz, 1H), 3.77-3.74 (m, 4H), 3.05 (s, 3H), 2.72 |
| | | (br s, 4H), 2.06-1.95 (m, 4H), 1.38 (br s, 4H), 0.26 (s, 4H) |
| Compound | 532.1 | (DMSO-d⁶) δ 10.06 (s, 1H), 8.92 (s, 1H), 7.69 (d, J = 7.5 Hz, 1H), |
| 18 | (M + H)⁺ | 7.55 (d, J = 8.6 Hz, 1H), 7.39 (d, J = 7.2 Hz, 1H), 7.08 (d, J = 2.2 Hz, |
| | | 1H), 7.02 (dd, J = 2.2, 8.6 Hz, 1H), 6.96 (d, J = 8.6 Hz, 1H), 3.78 (m, |
| | | 4H), 3.08 (s, 3H), 2.74 (m, J = 2.0 Hz, 2H), 2.54 (m, 2H), 2.05-1.96 |
| | | (m, 4H), 1.58-1.52 (m, 2H), 1.42-1.35 (m, 1H), 1.09 (m, 2H), 0.83 |
| | | (d, J = 6.4 Hz, 3H) |
| Compound | 568.3 | (DMSO-d⁶) δ 10.09 (m, 1 H) 8.89 (s, 1 H) 7.69 (m, 1 H) 7.55 (d, |
| 19 | (M + H)⁺ | J = 8.50 Hz, 1 H) 7.38 (d, J = 7.20 Hz, 1 H) 7.10 (d, J = 2.20 Hz, 1 H) 7.04 |
| | | (m, 1 H) 6.94 (d, J = 8.60 Hz, 1 H) 6.06-5.68 (m, H) 3.76 (br t, J = 5.50 |
| | | Hz, 4 H) 3.09 (s, 3 H) 2.86 (br d, J = 11.51 Hz, 2 H) 2.58 (br t, J = 11.10 |
| | | Hz, 2 H) 1.79-2.07 (m, 5 H) 1.64 (br d, J = 11.38 Hz, 2 H) 1.29 (m, 2 |
| | | H) |
| Compound | 586.1 | (DMSO-d⁶) δ 8.93 (s, 1H), 7.69 (dd, J = 7.6, 8.2 Hz, 1H), 7.57 (d, J = |
| 20 | (M + H)⁺ | 8.6 Hz, 1H), 7.39 (d, J = 7.2 Hz, 1H), 7.10-7.07 (m, 2H), 6.95 (d, J = |
| | | 8.6 Hz, 1H), 3.75 (br t, J = 5.7 Hz, 4H), 3.09 (s, 3H), 2.90-2.80 (m, |
| | | 2H), 2.66-2.57 (m, 2H), 2.44-2.35 (m, 1H), 2.05 1.90 (m, 4H), 1.76 |
| | | (br d, J = 11.0 Hz, 2H), 1.46-1.31 (m, 2H) |
| Compound | 559.3 | (DMSO-d⁶) δ 10.06 (br d, J = 6.25 Hz, 1H), 9.21 (s, 1H), 7.58 (d, |
| 21 | (M + H)⁺ | J = 8.63 Hz, 1H), 7.22 (s, 1H), 7.12 (d, J = 2.25 Hz, 1H), 7.08-6.99 (m, |
| | | 1H), 3.98 (br t, J = 5.50 Hz, 4H), 3.09 (s, 3H), 2.68 (br t, J = 4.88 Hz, |
| | | 4H), 2.40 (s, 3H), 2.08-1.93 (m, 4H), 1.28 (br s, 4H), 0.24 (s, 4H) |
| Compound | 523.2 | (DMSO-d⁶) δ 10.06 (s, 1 H), 9.02 (s, 1 H), 7.97-7.84 (m, 2H), 7.58 (d, |
| 22 | (M + H)⁺ | J = 8.8 Hz, 1 H), 7.44 (dd, J = 7.2, 1.2 Hz, 1H), 7.12 (d, J = 2.0 Hz, 1H), |
| | | 7.04 (dd, J = 8.4, 2.4 Hz, 1 H), 5.34 (d, J = 5.26 Hz, 1 H), 4.47 (t, J = 6.0 |
| | | Hz, 1H), 3.10 (s, 3H), 2.78-2.59 (m, 4 H), 2.38-2.23 (m, 1H), 1.66- |
| | | 1.38 (m, 8 H), 1.35-1.20 (m, 4H), 0.22 (s, 4H) |
| Compound | 558.1 | (DMSO-d⁶) δ 10.10 (br s, 1H), 8.93 (s, 1H), 7.98-7.94 (m, 1H), 7.91 |
| 23 | (M + H)⁺ | (d, J = 7.5 Hz, 1H), 7.42 (m,, 1H), 7.40 (m, 1H), 7.15 (m, 1H), 7.09 |
| | | (m, 1H), 3.73 (s, 2H), 3.08 (s, 3H), 2.66 (br d, J = 5.5 Hz, 4H), 2.57- |
| | | 2.62 (m, 4H), 1.93-2.02 (m, 4H), 1.25 (br s, 4H), 0.22 (s, 4H) |
| Compound | 512.2 | (DMSO-d⁶) δ 10.16-9.88 (m, 1H), 8.80-8.57 (m, 1H), 7.56-7.41 (m, |
| 25 | (M + H)⁺ | 1H), 7.18-6.59 (m, 2H), 6.78 (d, J = 3.3 Hz, 1H), 6.38 (d, J = 3.3 Hz, |
| | | 1H), 5.33 (d, J = 5.4 Hz, 1H), 4.44-4.25 (m, 1H), 3.09 (s, 3H), 2.67 (br |
| | | t, J = 5.1 Hz, 4H), 2.40-2.21 (m, 1H), 1.87-1.62 (m, 1H), 1.62-1.14 (m, |
| | | 10H), 0.24 (s, 4H) |
| Compound | 558.2 | (DMSO-d⁶) δ 12.12-12.50 (m, 1H), 9.69-10.14 (m, 1H), 8.03-8.11 |
| 26 | (M + H)⁺ | (m, 1H), 7.76-7.92 (m, 1H), 7.18-7.25 (m, 1H), 6.92-7.12 (m, 2H), |
| | | 3.90-4.07 (m, 4H), 3.01-3.10 (m, 3H), 2.77-2.92 (m, 4H), 2.27- |
| | | 2.33 (m, 3H), 1.92-2.12 (m, 4H), 1.08-1.71 (m, 4H), 0.27-0.42 (m, |
| | | 4H |
| Compound | 557.2 | (DMSO-d⁶) δ δ ppm 11.96-12.19 (m, 1 H) 9.83-9.96 (m, 1 H) 7.79- |
| 28 | (M + H)⁺ | 8.08 (m, 1 H) 7.62-7.72 (m, 1 H) 6.95-7.23 (m, 3 H) 6.60-6.70 (m, |
| | | 1 H) 3.68-3.87 (m, 4 H) 3.01-3.10 (m, 3 H) 2.78-2.96 (m, 4 H) |
| | | 2.27 (s, 3 H) 1.92-2.13 (m, 4 H) 1.43-1.73 (m, 4 H) 0.26-0.43 (m, 4 |
| | | H) |
| Compound | 554.2 | (DMSO-d⁶) δ 9.88 (br s, 1 H) 9.35-9.43 (m, 1 H) 8.02-8.17 (m, 1 H) |
| 29 | (M + H)⁺ | 7.79-7.90 (m, 1 H) 7.35-7.42 (m, 1 H) 7.13-7.19 (m, 1 H) 7.03- |
| | | 7.11 (m, 2 H) 3.79-3.88 (m, 4 H) 3.04 (s, 3 H) 2.78-2.91 (m, 4 H) |
| | | 1.98-2.15 (m, 4 H) 1.54 (br s, 4 H) 0.35 (s, 4 H) |
| Compound | 541.2 | (DMSO-d⁶) δ 12.22 (s, 1 H) 9.94 (br d, J = 5.75 Hz, 1 H) 8.11 (d, |
| 30 | (M + H)⁺ | J = 8.50 Hz, 1 H) 7.66 (s, 1 H) 7.58-7.48 (m, 1 H) 7.28-7.17 (m, 1 H) |
| | | 7.14-7.00 (m, 2 H) 6.37-6.27 (m, 1 H) 3.95 (br d, J = 10.88 Hz, 2 H) |
| | | 3.87-3.67 (m, 2 H) 3.11-3.00 (m, 3 H) 2.98-2.79 (m, 4 H) 2.76- |
| | | 2.63 (m, 2 H) 1.82-1.51 (m, 4 H) 0.48-0.25 (m, 4 H) |
| Compound | 529.1 | (DMSO-d⁶) δ 12.02-11.74 (m, 1H), 9.88-9.52 (m, 1H), 7.88-7.63 (m, |
| 32 | (M + H)⁺ | 1H), 7.59-7.41 (m, 1H), 7.37-7.19 (m, 1H), 7.06-6.67 (m, 4H), 6.31- |
| | | 6.10 (m, 1H), 4.42-4.27 (m, 1H), 4.11-3.89 (m, 1H), 3.00-2.80 (m, |
| | | 5H), 2.73-2.61 (m, 4H), 2.48-2.37 (m, 2H), 1.59-1.24 (m, 4H), 0.32- |
| | | 0.04 (m, 4H) |
| Compound | 558.3 | (CDCl₃) δ 12.31-12.04 (br s, 1 H), 8.44-8.29 (m, 2 H), 8.09 (br s, 1 H), |
| 34 | (M + H)⁺ | 7.76 (d, J = 7.6 Hz, 1 H), 7.53 (br t, J = 7.6 Hz, 1 H), 7.48 (br s, 1 H), |
| | | 7.28-7.32 (m, 1 H), 7.03 (br d, J = 7.6 Hz, 1 H), 6.59 (br s, 1 H), 4.56 |
| | | (s, 1 H), 3.07 (s, 3 H), 3.01 (br s, 4 H), 1.83-1.61 (m, 4 H), 1.28 (s, 9 |
| | | H), 0.45 (s, 4 H) |
| Compound | 573.1 | (DMSO-d⁶) δ 12.21-11.99 (m, 1 H), 8.32-7.99 (m, 1 H), 7.89-7.66 |
| 35 | (M + H)⁺ | (m, 2 H), 7.15-7.33 (m, 2 H), 7.09-6.94 (m, 1 H), 6.87-6.74 (m, 1 |
| | | H), 3.89-3.68 (m, 6 H), 3.21-3.21 (m, 2 H), 2.96-2.79 (m, 4 H), |
| | | 2.16-1.93 (m, 5 H), 1.71-1.48 (m, 4 H), 0.44-0.28 (m, 4 H) |
| Compound | 574.2 | (DMSO-d⁶) δ 10.36-9.58 (br s, 1H), 9.05 (s, 1H), 7.77-7.65 (m, 1H), |
| 45 | (M + H)⁺ | 7.58 (d, J = 8.4 Hz, 1H), 7.37 (d, J = 7.2 Hz, 1H), 7.12 (d, J = 2.4 Hz, 1H), |
| | | 7.04-6.98 (m, 1H), 6.96 (d, J = 8.40 Hz, 1H), 5.22-4.66 (m, 1H), 3.90- |
| | | 3.71 (m, 6H), 3.40-3.34 (m, 2H), 2.77-2.61 (m, 4H), 2.09-1.91 (m, |
| | | 4H), 1.42-1.20 (m, 4H), 0.25 (s, 4H) |
| Compound | 545.2 | (DMSO-d⁶) δ 10.85-10.69 (m, 1 H), 8.97-8.89 (m, 1 H), 7.82-7.74 |
| 46 | (M + H)⁺ | (m, 1 H), 7.72-7.65 (m, 1 H), 7.40-7.32 (m, 1 H), 7.00-6.91(m, 1 |
| | | H), 6.54-6.47 (m, 1 H), 3.85-3.74 (m, 4 H), 3.44-3.39 (m, 3 H), |
| | | 3.01-2.89 (m, 4 H), 2.08-1.89 (m, 4 H), 1.35-1.25 (m, 4 H), 0.31- |
| | | 0.17 (m, 4 H) |
| Compound | 589.2 | (DMSO-d⁶) δ 10.31-9.70 (m, 1H), 9.22 (s, 1H), 7.64-7.53 (m, 1H), |
| 47 | (M + H)⁺ | 7.26-7.20 (m, 1H), 7.16-7.12 (m, 1H), 7.08-7.01 (m, 1H), 5.21-4.73 |
| | | (m, 1H), 4.05-3.94 (m, 4H), 3.78 (t, J = 6.50 Hz, 2H), 2.68 (br s, 6H), |
| | | 2.41 (s, 3H), 2.08-1.94 (m, 4H), 1.34-1.23 (m, 4H), 0.31-0.18 (m, 4H) |
| Compound | 513.1 | (DMSO-d⁶) δ 8.77-8.58 (m, 1H), 7.79-7.61 (m, 1H), 6.89-6.70 (m, |
| 49 | (M + H)⁺ | 1H), 6.56-6.29 (m, 2H), 5.34 (d, J = 5.4 Hz, 1H), 4.42-4.20 (m, 1H), |
| | | 3.40 (s, 3H), 3.08-2.90 (m, 4H), 1.86-1.66 (m, 1H), 1.63-1.38 (m, 6H), |
| | | 1.34-1.16 (m, 6H), 0.37-0.17 (m, 4H) |
| Compound | 550.2 | (DMSO-d⁶) δ 10.02-10.13 (m, 1H), 8.66 (s, 1H), 7.50 (d, J = 8.6 Hz, |
| 50 | (M + H)⁺ | 1H), 7.29 (s, 1H), 7.10 (d, J = 2.0 Hz, 1H), 6.97-7.04 (m, 1H), 3.65 |
| | | (br dd, J = 5.3, 5.9 Hz, 4H), 3.09 (s, 3H), 2.62-2.76 (m, 4H), 2.06- |
| | | 2.21 (m, 4H), 1.19-1.35 (m, 4H), 0.25 (s, 4H) |
| Compound | 542.3 | (DMSO-d⁶) δ 8.62 (s, 1 H) 7.37-7.54 (m, 1 H) 7.10-7.22 (m, 1 H) |
| 51 | (M + H)⁺ | 6.95-7.08 (m, 1 H) 6.65-6.80 (m, 1 H) 6.23-6.42 (m, 1 H) 4.95- |
| | | 5.13 (m, 1 H) 4.27-4.48 (m, 1 H) 3.72-3.92 (m, 2 H) 3.22-3.42 (m, |
| | | 2 H) 2.66-2.77 (m, 4 H) 2.24-2.39 (m, 1 H) 1.68-1.83 (m, 1 H) |
| | | 1.45-1.67 (m, 6 H) 1.21-1.43 (m, 5 H) 0.27 (m, 4 H) |
| Compound | 532.0 | (DMSO-d⁶) δ 10.03-9.87 (m, 1 H), 8.82-8.76 (m, 1 H), 7.72-7.63 |
| 52 | (M + H) | (m, 1 H), 7.41-7.34 (m, 1 H), 7.32-7.26 (m, 1 H), 7.10-7.01 (m, 1 |
| | | H), 6.96-6.89 (m, 1 H), 6.88-6.79 (m, 1 H), 3.79-3.73 (m, 4 H), |
| | | 3.11-3.03 (m, 3 H), 2.96-2.87 (m, 4 H), 2.05-1.93 (m, 4 H), 1.45 |
| | | (br s, 8 H) |
| Compound | 530.3 | (DMSO-d⁶) δ 9.96 (br d, J = 1.76 Hz, 1 H) 8.82 (s, 1 H) 7.67 (dd, |
| 53 | (M + H)⁺ | J = 8.34, 7.47 Hz, 1 H) 7.37 (d, J = 7.28 Hz, 1 H) 7.26 (d. J = 8.40 Hz, 1 |
| | | H) 7.04 (d, J = 2.12 Hz, 1 H) 6.92 (d, J = 8.52 Hz, 1 H) 6.80 (m, 1 H) |
| | | 3.76 (br t, J = 5.52 Hz, 4 H) 3.08 (s, 3 H) 3.03 (s, 4 H) 2.30 (br t, J = 5.52 |
| | | Hz, 2 H) 1.89-2.05 (m, 6 H) 1.30-1.38 (m, 2 H) |
| Compound | 510.3 | (DMSO-d⁶) δ 9.03 (m, 1 H) 7.65-7.72 (m, 1 H) 7.56-7.61 (m, 1 H) |
| 54 | (M + H)⁺ | 7.34-7.38 (m, 1 H) 7.08-7.12 (m, 1 H) 6.99-7.04 (m, 1 H) 6.80- |
| | | 6.85 (m, 1 H) 3.68-3.78 (m, 4 H) 3.50-3.61 (m, 4 H) 3.07 (m, 3 H) |
| | | 2.65-2.73 (m, 4 H) 1.18-1.40 (m, 4 H) 0.17-0.32 (m, 4 H) |
| Compound | 522.3 | (DMSO-d⁶) δ 9.02-9.06 (m, 1 H) 7.66-7.73 (m, 1 H) 7.59-7.63 (m, |
| 55 | (M + H)⁺ | 1 H) 7.31-7.38 (m, 1 H) 7.08-7.14 (m, 1 H) 7.00-7.07 (m, 1 H) |
| | | 6.60-6.68 (m, 1 H) 4.69-4.78 (m, 2 H) 3.76-3.85 (m, 2 H) 3.59- |
| | | 3.67 (m, 2 H) 3.12-3.20 (m, 1 H) 3.06 (s, 3 H) 2.65-2.73 (m, 4 H) |
| | | 1.89-1.95 (m, 1 H) 1.35 (s, 4 H) 0.24 (s, 4 H) |
| Compound | 524.3 | (DMSO-d⁶) δ 10.05 (s, 1H), 9.06 (s, 1H), 7.58-7.75 (m, 2H), 7.35- |
| 56 | (M + H)⁺ | 7.36 (m, 1H), 7.12 (s, 1H), 7.03-7.05 (m, 1H), 6.82-6.84 (m, 1H), |
| | | 4.30-4.33 (m, 1H), 4.12-4.15 (m, 1H), 3.92-3.95(m, 1H), 3.59-3.62 |
| | | (m, 2H), 3.10 (s, 3H), 2.79-2.93 (m, 1H), 2.84-2.86 (m, 5H), 1.34 (s, |
| | | 4H), 1.22-1.12 (m, 3H), 0.26 (s, 4H) |
| Compound | 520.3 | (DMSO-d⁶) δ 10.05 (s, 1H), 8.97 (s, 1H), 7.58-7.65 (m, 2H), 7.27- |
| 57 | (M + H)⁺ | 7.34 (m, 1H), 7.10-7.15 (m, 1H), 7.01-7.07 (m, 1H), 6.33-6.38 (m, |
| | | 1H), 3.96 (s, 4H), 3.09 (s, 3H), 2.64-2.75 (m, 4H), 2.13-2.23 (m, |
| | | 4H), 1.78-1.88 (m, 2H), 1.32-1.43 (m, 4H), 0.28 (s, 4H) |
| Compound | 522.3 | (DMSO-d⁶) δ 9.82-10.32 (m, 1 H) 8.95 (s, 1 H) 7.53-7.70 (m, 2 H) |
| 58 | (M + H)⁺ | 7.35 (d, J = 7.13 Hz, 1 H) 7.12 (d, J = 2.25 Hz, 1 H) 7.03 (dd, J = 8.57, |
| | | 2.31 Hz, 1 H) 6.41 (d, J = 8.00 Hz, 1 H) 4.46 (t, J = 7.50 Hz, 2 H) 4.24 |
| | | (d, J = 10.51 Hz, 2 H) 4.01-4.12 (m, 2 H) 3.10 (s, 3 H) 2.87 (t, J = 7.44 |
| | | Hz, 2 H) 2.70 (br t, J = 5.13 Hz, 4 H) 1.36 (br s, 4 H) 0.28 (s, 4 H) |
| Compound | 545.3 | (DMSO-d⁶) δ 8.90 (s, 1 H) 8.11-8.27 (m, 1 H) 7.57-7.82 (m, 1 H) |
| 59 | (M + H)⁺ | 7.27-7.45 (m, 1 H) 6.83-7.07 (m, 1 H) 6.54-6.70 (m, 1 H) 4.06- |
| | | 4.13 (m, 1 H) 3.69-3.96 (m, 4 H) 3.13-3.21 (m, 3 H) 2.70-2.86 (m, |
| | | 4 H) 1.86-2.14 (m, 4 H) 1.14-1.39 (m, 4 H) 0.24 (s, 4 H) |
| Compound | 516.3 | (DMSO-d⁶) δ 10.04 (s, 1 H) 8.98 (s, 1 H) 7.75 (t, J = 7.84 Hz, 1 H) 7.58 |
| 60 | (M + H)⁺ | (d, J = 8.53 Hz, 1 H) 7.48 (d, J = 7.40 Hz, 1 H) 7.11 (d, J = 2.13 Hz, 1 H) |
| | | 6.97-7.07 (m, 1 H) 6.55-6.68 (m, 1 H) 4.36-4.53 (m, 4 H) 3.09 (s, 3 |
| | | H) 2.69 (br t, J = 5.02 Hz, 4 H) 1.33 (br s, 4 H) 0.25 (s, 4 H) |
| Compound | 530.2 | (DMSO-d⁶) δ 10.10 (s, 1 H) 8.98 (s, 1 H) 7.69-7.79 (m, 1 H) 7.53- |
| 61 | (M + H)⁺ | 7.61 (m, 1 H) 7.44-7.50 (m, 1 H) 7.09-7.17 (m, 1 H) 7.00-7.07 (m, |
| | | 1 H) 6.55-6.63 (m, 1 H) 4.33-4.53 (m, 4 H) 3.14-3.26 (m, 2 H) |
| | | 2.62-2.75 (m, 4 H) 1.33 (br s, 4 H) 1.21-1.33 (m, 3 H) 0.25 (s, 4 H) |
| Compound | 600.4 | (DMSO-d⁶) δ 9.07 (s, 1H), 7.65-7.73 (m, 1H), 7.55 (d, J = 8.5 Hz, |
| 62 | (M + H)⁺ | 1H), 7.37 (d, J = 7.4 Hz, 1H), 7.16 (d, J = 2.1 Hz, 1H), 7.00-7.07 (m, |
| | | 1H), 6.96 (d, J = 8.6 Hz, 1H), 3.75-3.85 (m, 6H), 2.66 (br t, J = 5.1 |
| | | Hz, 4H), 1.92-2.06 (m, 4H), 1.32 (br s, 4H), 1.09-1.16 (m, 2H), 0.95- |
| | | 1.03 (m, 2H), 0.25 (s, 4H) |
| Compound | 545.3 | (DMSO-d⁶) δ 9.85-10.22 (m, 1 H) 9.17 (s, 1 H) 8.52 (s, 1 H) 8.45 (s, 1 |
| 63 | (M + H)⁺ | H) 7.59 (d, J = 8.50 Hz, 1 H) 7.13 (d, J = 2.25 Hz, 1 H) 7.04 (dd, J = 8.57, |
| | | 2.31 Hz, 1 H) 3.87 (br t, J = 5.57 Hz, 4 H) 3.10 (s, 3 H) 2.62-2.73 (m, |
| | | 4 H) 1.96-2.15 (m, 4 H) 1.30 (br s, 4 H) 0.25 (s, 4 H) |
| Compound | 543.3 | (DMSO-d⁶) δ 9.78-9.91 (m, 1 H) 9.07 (s, 1 H) 7.84-7.90 (m, 1 H) |
| 64 | (M + H)⁺ | 7.43-7.50 (m, 2 H) 7.27-7.33 (m, 1 H) 7.09-7.18 (m, 2 H) 6.98- |
| | | 7.04 (m, 1 H) 3.45-3.54 (m, 4 H) 3.04 (s, 3 H) 2.83 (br t, J = 5.07 Hz, |
| | | 4 H) 2.00-2.14 (m, 4 H) 1.39-1.54 (m, 4 H) 0.31 (s, 4 H) |
| Compound | 573.3 | (DMSO-d⁶) δ 9.65-10.03 (m, 1 H) 9.01-9.11 (m, 1 H) 7.86 (d, |
| 65 | (M + H)⁺ | J = 8.44 Hz, 1 H) 7.40-7.53 (m, 2 H) 7.35 (s, 1 H) 7.08-7.19 (m, 2 H) |
| | | 7.01 (dd, J = 8.44, 2.08 Hz, 1 H) 4.82-5.07 (m, 1 H) 3.69-3.80 (m, 2 |
| | | H) 3.45-3.56 (m, 4 H) 3.23-3.30 (m, 2 H) 2.82 (br t, J = 5.01 Hz, 4 H) |
| | | 2.00-2.14 (m, 4 H) 1.37-1.56 (m, 4 H) 0.35 (s, 4 H) |
| Compound | 544.3 | (DMSO-d⁶) δ 9.89-9.92 (m, 1 H) 8.97 (s, 1 H) 8.26 (d, J = 6.13 Hz, 1 |
| 66 | (M + H)⁺ | H) 8.18 (s, 1 H) 7.51 (d, J = 8.50 Hz, 1 H) 7.05 (d, J = 2.13 Hz, 1 H) 6.97 |
| | | (dd, J = 8.57, 2.19 Hz, 1 H) 6.80 (d, J = 6.25 Hz, 1 H) 3.78-3.93 (m, 4 |
| | | H) 3.05 (s, 3 H) 2.65-2.75 (m, 4 H) 1.96-2.11 (m, 4 H) 1.28-1.44 |
| | | (m, 4 H) 0.26 (s, 4 H) |
| Compound | 563.3 | (DMSO-d⁶) δ 9.14 (s, 1H), 7.53 (d, J = 8.7 Hz, 1H), 7.22 (s, 1H), 7.08 |
| 67 | (M + H)⁺ | (d, J = 2.1 Hz, 1H), 6.99-7.03 (m, 1H), 3.98 (br t, J = 5.5 Hz, 4H), |
| | | 3.77 (t, J = 6.6 Hz, 2H), 3.30 (m, 2H), 2.61 (br s, 4H), 2.40 (s, 3H), |
| | | 1.92-2.06 (m, 4H), 1.42 (br s, 6H) |
| Compound | 562.4 | (DMSO-d⁶) δ 9.94-10.07 (m, 1H), 8.94 (s, 1H), 7.50-7.58 (m, 1H), |
| 68 | (M + H)⁺ | 7.22-7.27 (m, 1H), 7.05-7.09 (m, 1H), 6.98-7.04 (m, 1H), 6.78- |
| | | 6.83 (m, 1H), 4.96 (s, 1H), 3.71-3.82 (m, 6H), 3.29-3.32 (m, 2H), |
| | | 2.56-2.67 (m, 4H), 2.32 (s, 3H), 1.89-2.03 (m, 4H), 1.35-1.50 (m, |
| | | 6H). |
| Compound | 580.2 | (DMSO-d⁶) δ 10.01 (s, 1H), 8.65 (s, 1H), 7.48 (d, J = 8.6 Hz, 1H), |
| 69 | (M + H)⁺ | 7.27 (s, 1H), 7.10 (d, J = 2.3 Hz, 1H), 7.00 (dd, J = 2.3, 8.5 Hz, 1H), |
| | | 4.96 (t, J = 5.6 Hz, 1H), 3.77 (q, J = 6.4 Hz, 2H), 3.64 (br t, J = 5.6 Hz, |
| | | 4H), 2.66 (br t, J = 4.9 Hz, 6H), 1.98-2.20 (m, 4H), 1.27 (br s, 4H), |
| | | 0.24 (s, 4H) |
| Compound | 534.3 | (DMSO-d⁶) δ 8.85 (s, 1 H) 7.50-7.56 (m, 1 H) 7.36 (s, 1 H) 7.05- |
| 70 | (M + H)⁺ | 7.09 (m, 1 H) 6.97-7.03 (m, 1 H) 6.40-6.45 (m, 1 H) 4.32-4.48 (m, |
| | | 4 H) 3.74-3.81 (m, 2 H) 2.57-2.72 (m, 6 H) 2.34 (s, 3 H) 1.37-1.51 |
| | | (m, 6 H) |
| Compound | 575.4 | (DMSO-d⁶) δ 9.39 (s, 1 H) 8.04-8.09 (m, 1 H) 7.82-7.89 (m, 1 H) |
| 71 | (M + H)⁺ | 7.36-7.41 (m, 1 H) 7.13-7.1(m, 1 H) 7.02-7.12 (m, 2 H) 3.84 (br t, |
| | | J = 5.52 Hz, 4 H) 3.75 (t, J = 6.65 Hz, 2 H) 3.27-3.30 (m, 2 H) 2.78- |
| | | 2.90 (m, 4 H) 2.00-2.14 (m, 4 H) 1.42-1.67 (m, 4 H) 0.36 (s, 4 H) |
| Compound | 588.3 | (DMSO-d⁶) δ 9.96-10.09 (m, 1H), 9.02 (s, 1H), 7.55-7.62 (m, 1H), |
| 72 | (M + H)⁺ | 7.23-7.26 (m, 1H), 7.08-7.14 (m, 1H), 6.98-7.04 (m, 1H), 6.78- |
| | | 6.83 (m, 1H), 4.89-5.02 (m, 1H), 3.71-3.83 (m, 6H), 3.32-3.35 (m, |
| | | 2H), 2.63-2.71 (m, 4H), 2.33 (s, 3H), 1.91-2.06 (m, 4H), 1.23-1.35 |
| | | (m, 4H), 0.19-0.27 (m, 4H) |
| Compound | 587.3 | (DMSO-d⁶) δ 9.15 (s, 1 H) 7.54-7.62 (m, 1 H) 7.17-7.22 (m, 1 H) |
| 73 | (M + H)⁺ | 7.10-7.14 (m, 1 H) 6.98-7.07 (m, 1 H) 3.93-4.11 (m, 2 H) 3.72- |
| | | 3.85 (m, 4 H) 3.33 (br d, J = 1.13 Hz, 2 H) 2.63-2.72 (m, 6 H) 2.38 (s, |
| | | 3 H) 1.31 (s, 4 H) 0.25 (s, 4 H) |
| Compound | 561.3 | (DMSO-d⁶) δ 9.86-10.26 (m, 1H), 9.04 (s, 1H), 7.51-7.56 (m, 1H), |
| 74 | (M + H)⁺ | 7.19-7.22 (m, 1H), 7.06-7.11 (m, 1H), 6.98-7.04 (m, 1H), 4.76- |
| | | 5.17 (m, 1H), 3.93-4.05 (m, 2H), 3.77 (br t, J = 6.6 Hz, 4H), 3.32- |
| | | 3.35 (m, 2H), 2.57-2.72 (m, 6H), 2.38 (s, 3H), 1.43 (br s, 6H) |
| Compound | 577.4 | (DMSO-d⁶) δ 9.61-10.32 (m, 1H), 9.02 (s, 1H), 7.16-7.37 (m, 2H), |
| 75 | (M + H)⁺ | 7.07 (d, J = 2.0 Hz, 1H), 6.83 (dd, J = 2.1, 6.4 Hz, 1H), 4.69-5.31 (m, |
| | | 1H), 3.88-4.09 (m, 4H), 3.78 (t, J = 6.8 Hz, 2H), 2.80-3.02 (m, 4H), |
| | | 2.39 (s, 3H), 1.89-2.10 (m, 4H), 1.32-1.60 (m, 8H |
| Compound | 536.3 | (DMSO-d⁶) δ 9.69-10.37 (m, 1H), 8.65 (s, 1H), 7.91 (d, J = 2.3 Hz, |
| 76 | (M + H)⁺ | 1H), 7.45 (d, J = 8.5 Hz, 1H), 7.07 (d, J = 2.3 Hz, 1H), 6.99 (dd, J = |
| | | 2.3, 8.6 Hz, 1H), 6.68 (d, J = 2.4 Hz, 1H), 5.22-4.78 (m, 1H), 4.54- |
| | | 4.43 (m, 1H), 3.77 (t, J = 6.6 Hz, 2H), 3.34 (m, 2H), 2.61 (br s, 4H), |
| | | 1.97-2.24 (m, 8H), 1.41 (br s, 6H) |
| | | (CDCl₃) δ 8.53 (s, 1H), 7.44-7.61 (m, 2H), 7.08-7.24 (m, 2H), 7.00 |
| | | (br d, J = 7.8 Hz, 1H), 6.81 (br s, 1H), 4.36 (br t, J = 9.5 Hz, 1H), 4.17 |
| | | (t, J = 5.1 Hz, 2H), 3.28-3, 45 (m, 2H), 2.99-3.26 (m, 1H), 2.72 (br s, |
| | | 4H), 2.11-2.38 (m, 6H), 1.90-2.06 (m, 2H), 1.38-1.57 (m, 6H |
| Compound | 512.2 | (DMSO-d⁶) δ 8.51 (s, 1H), 7.60-7.64 (m, 1H), 7.26-7.32 (m, 1H), |
| 77 | (M + H)⁺ | 6.90-6.97 (m, 1H), 6.80-6.87 (m, 1H), 6.46-6.51 (m, 1H), 3.97- |
| | | 4.07 (m, 2H), 3.55-3.65 (m, 2H), 3.10-3.20 (m, 5H), 2.56-2.69 (m, |
| | | 1H), 2.32-2.36 (m, 1H), 1.76-1.91 (m, 2H), 1.51-1.72 (m, 4H), |
| | | 1.10 (s, 4H), 0.08 (s, 4H) |
| Compound | 592.3 | (DMSO-d⁶) δ 9.11 (s, 1 H) 7.55-7.61 (m, 1 H) 7.09-7.17 (m, 2 H) |
| 78 | (M + H)⁺ | 7.00-7.07 (m, 1 H) 6.82-6.91 (m, 1 H) 3.68-3.91 (m, 6 H) 3.28- |
| | | 3.32 (m, 2 H) 2.59-2.74 (m, 4 H) 1.89-2.10 (m, 4 H) 1.30 (s, 4 H) |
| | | 0.25 (s, 4 H) |
| Compound | 592.4 | (DMSO-d⁶) δ 9.11 (s, 1 H) 7.55-7.61 (m, 1 H) 7.09-7.17 (m, 2 H) |
| 79 | (M + H)⁺ | 7.00-7.07 (m, 1 H) 6.82-6.91 (m, 1 H) 3.68-3.91 (m, 6 H) 3.28- |
| | | 3.32 (m, 2 H) 2.59-2.74 (m, 4 H) 1.89-2.10 (m, 4 H) 1.30 (s, 4 H) |
| | | 0.25 (s, 4 H) |
| Compound | 598.3 | (DMSO-d⁶) δ 10.07 (s, 1H), 9.04 (s, 1H), 7.56-7.62 (m, 1H), 7.25 (s, |
| 80 | (M + H)⁺ | 1H), 7.11-7.18 (m, 1H), 7.02-7.10 (m, 1H), 6.78-6.83 (m, 1H), |
| | | 3.74-3.77 (m, 4H), 3.30-3.37 (m, 4H), 2.77 (br t, J = 4.6 Hz, 4H), |
| | | 2.32 (s, 3H), 1.95 (m, 8H) |
| Compound | 530.2 | (DMSO-d⁶) δ 10.02 (s, 1H), 8.60 (s, 1H), 7.49 (d, J = 8.5 Hz, 1H), |
| 81 | (M + H)⁺ | 7.08-7.15 (m, 2H), 6.96-7.05 (m, 1H), 4.93-5.02 (m, 1H), 3.72- |
| | | 3.84 (m, 2H), 3.43 (br t, J = 6.5 Hz, 4H), 3.36 (m, 2H) 2.67 (br t, J = |
| | | 5.1 Hz, 4H), 1.96-2.05 (m, 4H), 1.28 (br s, 4H), 0.25 (s, 4H) |
| Compound | 498.3 | (DMSO-d⁶) δ 10.23-9.81 (m, 1H), 8.69 (s, 1H), 7.93 (d, J = 2.3 Hz, |
| 82 | (M + H)⁺ | 1H), 7.46 (d, J = 8.6 Hz, 1H), 7.10 (d, J = 2.0 Hz, 1H), 7.00 (m, 1H), |
| | | 6.68 (d, J = 2.3 Hz, 1H), 4.89 (m, 1H), 3.78 (m, 2H), 3.35 (m, 2H), |
| | | 2.67 (m, 4H), 2.53 (br s, 2H), 2.42 (m, 2H), 1.81 (m, 2H), 1.26 (br s, |
| | | 4H), 0.24 (s, 4H) |
| Compound | 512.4 | (DMSO-d⁶) δ 8.64-8.72 (m, 1 H) 7.81-7.90 (m, 1 H) 7.42-7.49 (m, |
| 83 | (M + H)⁺ | 1 H) 7.06-7.13 (m, 1 H) 6.95-7.03 (m, 1 H) 6.59-6.67 (m, 1 H) |
| | | 4.69-4.81 (m, 1 H) 3.72-3.85 (m, 2 H) 3.28-3.31 (m, 2 H) 2.61- |
| | | 2.73 (m, 4 H) 2.04-2.18 (m, 2 H) 1.90-2.03 (m, 2 H) 1.76-1.89 (m, |
| | | 2 H) 1.59-1.73 (m, 2 H) 1.18-1.35 (m, 4 H) 0.17-0.31 (m, 4 H |
| Compound | 562.4 | (DMSO-d⁶) δ 9.60-10.43 (m, 1H), 8.69 (s, 1H), 7.91 (d, J = 2.0 Hz, |
| 84 | (M + H)⁺ | 1H), 7.46 (d, J = 8.4 Hz, 1H), 7.11 (br d, J = 1.6 Hz, 1H), 7.00 (dd, J = |
| | | 1.6, 8.4 Hz, 1H), 6.68 (s, 1H), 4.74-5.23 (m, 1H), 4.40-4.59 (m, 1H), |
| | | 3.78 (br t, J = 6.4 Hz, 2H), 3.31-3.49 (m, 2H), 2.67 (br s, 4H), 1.92- |
| | | 2.26 (m, 8H), 1.26 (br s, 4H), 0.24 (s, 4H) |
| Compound | 588.4 | (DMSO-d⁶) δ 9.01-9.10 (s, 1H), 7.66-7.76 (m, 1H), 7.53-7.58 (m, |
| 85 | (M + H)⁺ | 1H), 7.34-7.39 (m, 1H), 7.12-7.15 (m, 1H), 7.01-7.06 (m, 1H), |
| | | 6.94-7.00 (m, 1H), 3.73-3.94 (m, 5H), 3.44-3.54 (m, 2H), 3.22- |
| | | 3.28 (m, 1H), 2.65-2.72 (m, 4H), 1.90-2.12 (m, 4H), 1.30 (br d, J = |
| | | 6.8 Hz, 7H), 0.25 (s, 4H) |
| Compound | 554.3 | (DMSO-d⁶) δ 10.03 (br s, 1 H) 8.60 (s, 1 H) 7.46 (d, J = 8.20 Hz, 1 H) |
| 86 | (M + H)⁺ | 7.25-7.31 (m, 1 H) 7.07 (d, J = 2.40 Hz, 1 H) 6.95-7.02 (m, 1 H) 4.98 |
| | | (br s, 1 H) 3.72-3.84 (m, 2 H) 3.59-3.69 (m, 4 H) 3.28-3.31 (m, 2 |
| | | H) 2.56-2.65 (m, 4 H) 2.04-2.21 (m, 4 H) 1.43 (br s, 6 H) |
| Compound | 589.3 | (DMSO-d⁶) δ 9.46 (s, 1H), 8.08 (d, J = 8.4 Hz, 1H), 7.29 (s, 1H), 7.17 |
| 87 | (M + H)⁺ | (d, J = 1.9 Hz, 1H), 7.05 (dd, J = 1.8, 8.5 Hz, 1H), 3.96-4.06 (m, 4H), |
| | | 3.68-3.81 (m, 2H), 3.23-3.29 (m, 2H), 2.78-2.89 (m, 4H), 2.46 (s, |
| | | 3H), 1.99-2.13 (m, 4H), 1.45-1.63 (m, 4H), 0.36 (s, 4H) |
| Compound | 575.4 | (DMSO-d⁶) δ 8.99 (s, 1H), 7.15-7.23 (m, 2H), 6.72-6.76 (m, 1H), |
| 88 | (M + H)⁺ | 6.62-6.68 (m, 1H), 3.93-4.00 (m, 4H), 3.73-3.80 (m, 2H), 3.29- |
| | | 3.31 (m, 2H), 2.89-2.96 (m, 2H), 2.69-2.74 (m, 2H), 2.38 (s, 3H), |
| | | 1.93-2.06 (m, 4H), 1.65-1.71 (m, 2H), 0.43-0.51 (m, 4H) |
| Compound | 577.2 | (DMSO-d⁶) δ 9.11 (s, 1H), 7.53-7.61 (m, 1H), 7.22 (s, 1H), 7.10- |
| 89 | (M + H)⁺ | 7.14 (m, 1H), 7.01-7.07 (m, 1H), 3.96-4.04 (m, 4H), 3.78-3.83 (m, |
| | | 2H), 3.33-3.36 (m, 2H), 2.71-2.79 (m, 2H), 2.54 (br s, 1H), 2.41 (s, |
| | | 3H), 1.96-2.10 (m, 4H), 1.52-1.61 (m, 2H), 1.34-1.46 (m, 1H), |
| | | 1.00-1.13 (m, 2H), 0.81-0.89 (m, 3H) |
| Compound | 553.4 | (DMSO-d⁶) δ 9.96-10.11 (m, 1H), 8.96-9.07 (m, 1H), 7.84-7.97 |
| 90 | (M + H)⁺ | (m, 2H), 7.54-7.60 (m, 1H), 7.41-7.47 (m, 1H), 7.09-7.18 (m, 1H), |
| | | 6.99-7.06 (m, 1H), 5.26-5.39 (m, 1H), 4.89-5.03 (m, 1H), 4.39- |
| | | 4.53 (m, 1H), 3.69-3.87 (m, 2H), 3.34-3.37 (m, 2H), 2.57-2.77 (m, |
| | | 4H), 2.26-2.38 (m, 1H), 1.40-1.69 (m, 8H), 1.17-1.36 (m, 4H), |
| | | 0.12-0.30 (m, 4H) |
| Compound | 589.4 | (DMSO-d⁶) δ 10.06-9.88 (m, 1H), 9.17-9.14 (m, 1H), 7.55-7.47 |
| 91 | (M + H)⁺ | (m, 1H), 7.24-7.20 (m, 1H), 7.16-7.11 (m, 1H), 7.06-7.00 (m, 1H), |
| | | 5.11-4.89 (m, 1H), 4.01-3.94 (m, 4H), 3.81-3.76 (m, 2H), 2.62- |
| | | 2.57 (m, 4H), 2.41-2.39 (m, 3H), 2.05-1.94 (m, 4H), 1.48-1.38 (m, |
| | | 6H), 1.15-1.09 (m, 2H), 1.01-0.96 (m, 2H) |
| Compound | 540.2 | (DMSO-d⁶) δ 9.06 (1 H, s) 7.62 (1 H, d, J = 3.75 Hz) 7.51 (1 H, d, |
| 92 | (M + H)⁺ | J = 8.51 Hz) 7.09-7.16 (2 H, m) 7.02 (1 H, m) 4.90-5.08 (1 H, m) |
| | | 3.78 (2 H, t, J = 6.57 Hz) 3.56-3.69 (1 H, m) 3.36 (1 H, br s) 3.30 (1 H, |
| | | br d, J = 1.50 Hz) 2.68 (4 H, br t, J = 5.07 Hz) 1.87-2.00 (2 H, m) 1.71- |
| | | 1.83 (2 H, m) 1.58-1.70 (4 H, m) 1.23-1.33 (4 H, m) 0.25 (4 H, s) |
| Compound | 598.4 | (DMSO-d⁶) δ 10.15 (s, 1 H) 8.75 (s, 1 H) 7.45-7.57 (m, 1 H) 7.08- |
| 93 | (M + H)⁺ | 7.15 (m, 1 H) 6.97-7.05 (m, 1 H) 4.95 (s, 1 H) 3.75-3.81 (m, 2 H) |
| | | 3.53-3.60 (m, 4 H) 3.32-3.37 (m, 2 H) 2.69-2.69 (m, 1 H) 2.64- |
| | | 2.69 (m, 3 H) 2.06-2.18 (m, 4 H) 1.22-1.32 (m, 4 H) 0.28 (s, 4 H) |
| Compound | 580.2 | (DMSO-d⁶) δ 8.96 (s, 1 H) 7.49 (d, J = 8.50 Hz, 1 H) 7.11 (d, J = 2.13 |
| 94 | (M + H)⁺ | Hz, 1 H) 7.01 (dd, J = 8.57, 2.19 Hz, 1 H) 6.49 (s, 1 H) 3.78 (t, J = |
| | | 6.57 Hz, 2 H) 3.43-3.52 (m, 5 H) 3.31-3.32 (m, 2 H) 2.67 (br t, J = |
| | | 5.00 Hz, 4 H) 2.00-2.16 (m, 4 H) 1.25 (br d, J = 4.88 Hz, 4 H) 0.24 |
| | | (s, 4 H) |
| Compound | 588.3 | (DMSO-d⁶) δ 9.8-10.0 (m, 1H), 9.4-9.4 (m, 1H), 8.0-8.1 (m, 1H), 7.3- |
| 95 | (M + H)⁺ | 7.3 (m, 1H), 7.1-7.2 (m, 1H), 7.0-7.1 (m, 1H), 6.9-7.0 (m, 1H), 4.8-5.1 |
| | | (m, 1H), 3.8-3.9 (m, 4H), 3.7-3.8 (m, 2H), 3.3-3.3 (m, 2H), 2.8-2.9 (m, |
| | | 4H), 2.4-2.4 (m, 3H), 2.0-2.1 (m, 4H), 1.4-1.7 (m, 4H), 0.3-0.4 (m, |
| | | 4H) |
| Compound | 575.4 | (DMSO-d⁶) δ 9.38 (s, 1 H) 8.58 (d, J = 2.80 Hz, 2 H) 8.06 (d, J = 8.40 |
| 96 | (M + H)⁺ | Hz, 1 H) 7.16 (d, J = 2.00 Hz, 1 H) 7.01-7.09 (m, 1 H) 3.85-3.97 (m, |
| | | 4 H) 3.70-3.80 (m, 2 H) 3.24-3.29 (m, 2 H) 2.78-2.90 (m, 4 H) |
| | | 2.04-2.19 (m, 4 H) 1.54 (br s, 4 H) 0.35 (s, 4 H) |
| Compound | 587.2 | (DMSO-d⁶) δ 9.5-9.5 (m, 1H), 8.1-8.1 (m, 1H), 7.3-7.3 (m, 1H), 7.1- |
| 97 | (M + H)⁺ | 7.2 (m, 1H), 7.0-7.1 (m, 1H), 4.0-4.1 (m, 2H), 3.8-3.9 (m, 2H), 3.7-3.8 |
| | | (m, 2H), 3.3-3.3 (m, 2H), 2.8-2.9 (m, 4H), 2.7-2.8 (m, 2H), 2.4-2.5 (m, |
| | | 3H), 1.5-1.7 (m, 4H), 0.3-0.4 (m, 4H) |
| Compound | 539.3 | (DMSO-d⁶) δ 9.92-10.15 (m, 1 H) 9.11 (s, 1 H) 7.59 (d, J = 8.51 Hz, |
| 98 | (M + H)⁺ | 1 H) 7.08-7.16 (m, 2 H) 7.03 (dd, J = 8.51, 2.13 Hz, 1 H) 4.88-5.10 |
| | | (m, 1 H) 3.78 (t, J = 6.50 Hz, 2 H) 3.54 (br s, 4 H) 3.34-3.38 (m, 4 H) |
| | | 2.67 (br t, J = 4.94 Hz, 4 H) 2.36 (s, 3 H) 1.89-1.99 (m, 4 H) 1.31 (br |
| | | s, 4 H) 0.25 (s, 4 H) |
| Compound | 551.4 | (DMSO-d⁶) δ 9.12 (s, 1 H) 8.17 (s, 1 H) 7.58 (d, J = 8.63 Hz, 1 H) |
| 99 | (M + H)⁺ | 7.09-7.16 (m, 2 H) 7.03 (dd, J = 8.57, 2.31 Hz, 1 H) 3.81-3.95 (m, 2 |
| | | H) 3.78 (t, J = 6.50 Hz, 2 H) 3.48 (br d, J = 11.13 Hz, 2 H) 3.35 (br s, |
| | | 2 H) 2.68 (br t, J = 5.00 Hz, 4 H) 2.35 (s, 3 H) 1.61-1.70 (m, 2 H) |
| | | 1.32 (br s, 4 H) 0.74 (br d, J = 4.25 Hz, 1 H) 0.26 (s, 4 H) 0.16 (d, J = |
| | | 4.13 Hz, 1 H) |
| Compound | 539.4 | (DMSO-d⁶) δ 9.04-9.12 (m, 1 H) 7.73-7.85 (m, 1 H) 7.57-7.65 (m, |
| 100 | (M + H)⁺ | 2 H) 7.13 (s, 1 H) 7.00-7.07 (m, 1 H) 6.73 (d, J = 8.00 Hz, 1 H) 5.40- |
| | | 5.51 (m, 1 H) 3.73-3.83 (m, 2 H) 3.30-3.33 (m, 2 H) 2.61-2.75 (m, |
| | | 4 H) 1.99-2.15 (m, 2 H) 1.52-1.84 (m, 6 H) 1.34 (br s, 4 H) 0.24 (s, |
| | | 4 H) |
| Compound | 533.4 | (DMSO-d⁶) δ 9.16 (s, 1 H) 7.58 (d, J = 8.50 Hz, 1 H) 7.12 (d, J = 2.25 |
| 101 | (M + H)⁺ | Hz, 1 H) 7.09 (s, 1 H) 7.00-7.13 (m, 1 H) 7.02 (dd, J = 8.63, 2.25 Hz, 1 |
| | | H) 3.75-3.85 (m, 6 H) 3.36 (s, 1 H) 3.34-3.38 (m, 1 H) 3.28-3.29 |
| | | (m, 1 H) 2.67 (br t, J = 5.00 Hz, 4 H) 2.36 (s, 3 H) 2.07 (s, 1 H) 1.59- |
| | | 1.68 (m, 2 H) 1.49-1.58 (m, 4 H) 1.30 (br s, 4 H) 0.24 (s, 4 H) |
| Compound | 557.2 | (DMSO-d⁶) δ 9.14 (s, 1 H), 7.59 (d, J = 8.66 Hz, 1 H), 7.18 (s, 1 H), |
| 102 | (M + H)⁺ | 7.13 (d, J = 2.26 Hz, 1 H), 7.03 (dd, J = 8.60, 2.20 Hz, 1 H), 5.39-5.52 (d, |
| | | J = 52 Hz, 1 H), 3.74-3.94 (m, 4 H), 3.50-3.66 (m, 1 H), 3.33-3.36 |
| | | (m, 2 H), 2.68 (br t, J = 5.21 Hz, 4 H), 2.39 (s, 3 H), 2.19-2.32 (m, 2 |
| | | H), 1.27-1.38 (m, 4 H), 1.24 (s, 2 H), 0.25 (s, 4 H) |
| Compound | 589.2 | (DMSO-d⁶) δ 9.13 (s, 1H), 7.58 (d, 1H, J = 8.5 Hz), 7.16 (s, 1H), 7.13 |
| 103 | (M + H)⁺ | (d, 1H, J = 2.1 Hz), 7.03 (dd, 1H, J = 2.2, 8.6 Hz), 6.0-6.4 (m, 1H), 3.7- |
| | | 3.8 (m, 4H), 3.5-3.6 (m, 2H), 3.3-3.4 (m, 1H), 3.2-3.3 (m, 1H), 2.85 |
| | | (dt, 1H, J = 6.9, 13.8 Hz), 2.67 (br t, 4H, J = 4.9 Hz), 2.54 (br s, 1H), |
| | | 2.38 (s, 3H), 2.1-2.2 (m, 1H), 1.9-2.0 (m, 1H), 1.30 (br s, 4H), 0.25 (s, |
| | | 4H) |
| Compound | 612.4 | (DMSO-d⁶) δ 9.83-10.22 (m, 1 H), 8.85 (s, 1 H), 7.53 (d, J = 8.50 Hz, |
| 104 | (M + H)⁺ | 1 H), 7.25 (s, 1 H), 7.10 (d, J = 2.13 Hz, 1 H), 7.03 (m, 1 H), 6.79 (s, 1 |
| | | H), 5.67-6.05 (m, 1 H), 4.80-5.13 (m, 1 H), 3.67-3.83 (m, 6 H), |
| | | 3.34 (br s, 2 H), 2.84 (br d, J = 11.63 Hz, 2 H), 2.53-2.65 (m, 1 H), |
| | | 2.32 (s, 3 H), 1.77-2.07 (m, 5 H), 1.56-1.70 (m, 2 H), 1.20-1.37 (m, |
| | | 2 H) |
| Compound | 584.4 | (DMSO-d⁶) δ 9.69-9.98 (m, 1 H), 9.38 (s, 1 H), 8.07 (d, J = 8.38 Hz, 1 |
| 105 | (M + H)⁺ | H), 7.24-7.37 (m, 1 H), 7.16-7.23 (m, 1 H), 7.02-7.13 (m, 1 H), |
| | | 6.92-6.98 (m, 1 H), 3.72-3.92 (m, 4 H), 2.84 (br s, 4 H), 2.64-2.70 |
| | | (m, 1 H), 2.38 (s, 3 H), 2.01-2.12 (m, 4 H), 1.39-1.68 (m, 4 H), 0.90- |
| | | 1.05 (m, 4 H), 0.24-0.50 (m, 4 H) |
| Compound | 573.5 | (DMSO-d⁶) δ 9.94-10.35 (m, 1 H) 9.21 (s, 1 H) 7.56 (d, J = 8.8 Hz, 1 |
| 106 | (M + H)⁺ | H) 7.21 (s, 1 H) 7.13 (d, J = 2.0 Hz, 1 H) 7.02 (dd, J = 6.4, 2.19 Hz, 1 H) |
| | | 3.98 (br t, J = 5.6 Hz, 4 H) 3.19 (q, J = 7.2 Hz, 2 H) 2.67 (br t, J = 5.2 Hz, |
| | | 4 H) 2.40 (s, 3 H) 1.93-2.06 (m, 4 H) 1.28 (br s, 4 H) 1.22 (t. J = 7.2 |
| | | Hz, 3 H) 0.20-0.30 (m, 4 H) |
| Compound | 574.5 | (DMSO-d⁶) δ 8.98-9.03 (m, 1 H), 7.55-7.62 (m, 1 H), 7.19-7.23 |
| 107 | (M + H)⁺ | (m, 1 H), 7.10-7.14 (m, 1 H), 6.99-7.06 (m, 1 H), 6.35-6.40 (m, 1 |
| | | H), 5.47-5.56 (m, 1 H), 5.34-5.43 (m, 1 H), 3.82-3.95 (m, 2 H), |
| | | 3.74-3.81 (m, 2 H), 3.59-3.72 (m, 2 H), 3.33-3.36 (m, 2 H), 2.64- |
| | | 2.75 (m, 4 H), 2.27-2.38 (m, 3 H), 1.25-1.42 (m, 4 H), 0.19-0.31 |
| | | (m, 4 H) |
| Compound | 584.4 | (DMSO-d⁶) δ 9.81-10.03 (m, 1 H), 9.34-9.45 (m, 1 H), 8.06 (d, |
| 108 | (M + H)⁺ | J = 8.51 Hz, 1 H), 7.24-7.36 (m, 1 H), 7.13-7.19 (m, 1 H), 7.02-7.10 |
| | | (m, 1 H), 6.92-6.98 (m, 1 H), 3.76-3.86 (m, 4 H), 3.07-3.19 (m, 3 |
| | | H), 2.78-2.90 (m, 4 H), 2.38 (s, 3 H), 1.98-2.12 (m, 4 H), 1.40-1.66 |
| | | (m, 4 H), 1.14-1.27 (m, 4 H), 0.35 (s, 4 H) |
| Compound | 553.3 | (DMSO-d⁶) δ 9.75-10.38 (m, 1 H) 9.21 (s, 1 H) 7.59 (s, 1 H) 7.57 (d, |
| 109 | (M + H)⁺ | J = 8.63 Hz, 1 H) 7.12 (d, J = 2.13 Hz, 1 H) 7.03 (dd, J = 8.57, 2.19 Hz, |
| | | 1 H) 5.39-5.46 (m, 1 H) 4.74-5.19 (m, 1 H) 3.78 (t, J = 6.57 Hz, 2 H) |
| | | 3.32-3.37 (m, 2 H) 2.67 (br t, J = 5.07 Hz, 4 H) 2.47 (s, 3 H) 1.95- |
| | | 2.08 (m, 2 H) 1.68-1.82 (m, 4 H) 1.54-1.67 (m, 2 H) 1.29 (br s, 4 H) |
| | | 0.24 (s, 4 H) |
| Compound | 573.5 | (DMSO-d⁶) δ 10.08-9.74 (m, 1H), 9.14 (s, 1H), 8.43 (t, J = 1.8 Hz, |
| 110 | (M + H)⁺ | 1H), 8.17 (dd, J = 1.2, 8.1 Hz, 1H), 7.82-7.98 (m, 3H), 7.78 (s, 1H), |
| | | 7.16 (d, J = 2.0 Hz, 1H), 7.03 (dd, J = 2.0, 8.4 Hz, 1H), 3.16 (q, J = 7.3 |
| | | Hz, 2H), 2.83 (br t, J = 4.9 Hz, 4H), 1.47 (br s, 4H), 1.21 (t, J = 7.4 Hz, |
| | | 3H), 1.15 (s, 9H), 0.31 (s, 4H) |
| Compound | 627.2 | (CDCl₃) δ 8.74-8.75 (m, 1 H), 8.51 (s, 1 H), 8.31 (d, J = 8.25 Hz, 1 H), |
| 111 | (M + H)⁺ | 8.27 (dd, J = 5.57, 2.69 Hz, 1 H), 8.09 (dt, J = 8.57, 3.53 Hz, 1 H), 7.50 |
| | | (t, J = 8.94 Hz, 1 H), 6.80 (d, J = 8.25 Hz, 1 H), 4.41-4.48 (m, 4 H), |
| | | 4.15 (br s, 2 H), 3.63-3.70 (m, 2 H), 3.14-3.21 (m, 4 H), 2.73-2.89 |
| | | (m, 1 H), 1.45-1.49 (m, 4 H), 0.37 (s, 4 H). |
| Compound | 627.2 | (DMSO-d⁶) δ 9.18 (s, 1 H), 8.34-8.45 (m, 2 H), 8.04-8.27 (m, 1 H), |
| 112 | (M + H)⁺ | 7.90 (t, J = 9.32 Hz, 1 H), 6.72 (s, 1 H), 4.77-4.98 (m, 1 H), 4.50 (t, |
| | | J = 12.57 Hz, 4 H), 3.78 (br t, J = 6.63 Hz, 2 H), 3.39-3.69 (m, 2 H), |
| | | 3.01 (br s, 4 H), 1.41 (br s, 4 H), 0.31 (s, 4 H) |
| Compound | 539.4 | (DMSO-d⁶) δ 9.97-9.81 (1 H, m) 9.37 (1 H, s) 8.10 (1 H, d, J = 8.53 |
| 113 | (M + H)⁺ | Hz) 7.99 (1 H, t, J = 7.97 Hz) 7.71 (1 H, d, J = 7.40 Hz) 7.18 (1 H, d, J = |
| | | 2.01 Hz) 7.07 (1 H, m) 6.91 (1 H, d, J = 8.28 Hz) 5.45-5.55 (1 H, |
| | | m) 4.88-5.00 (1 H, m) 3.76 (2 H, t, J = 6.46 Hz) 3.28-3.31 (2 H, m) |
| | | 2.81-2.93 (4 H, m) 2.04-2.16 (2 H, m) 1.46-1.92 (10 H, m) 0.35 (4 |
| | | H, s) |
| Compound | 588.5 | (DMSO-d⁶) δ 9.18 (s, 1 H) 7.81 (d, J = 8.40 Hz, 1 H) 7.24 (br s, 1 H) |
| 114 | (M + H)⁺ | 7.17 (br s, 1 H) 7.11 (d, J = 2.00 Hz, 1 H) 6.97-7.02 (m, 1 H) 3.68- |
| | | 3.88 (m, 6 H) 3.25-3.28 (m, 2 H) 2.75-2.88 (m, 4 H) 2.44 (s, 3 H) |
| | | 1.89-2.13 (m, 4 H) 1.35-1.53 (m, 4 H) 0.29 (s, 4 H) |
| Compound | 575.5 | (DMSO-d⁶) δ 9.89-10.13 (m, 1 H), 8.97-9.06 (m, 1 H), 7.54-7.64 |
| 115 | (M + H)⁺ | (m, 1 H), 7.19-7.26 (m, 1 H), 7.09-7.16 (m, 1 H), 6.99-7.07 (m, 1 |
| | | H), 6.38-6.45 (m, 1 H), 5.51-5.61 (m, 1 H), 5.37-5.48 (m, 1 H), |
| | | 4.89-5.06 (m, 1 H), 3.68-3.98 (m, 6 H), 3.35 (s, 2 H), 2.69 (s, 4 H), |
| | | 2.31-2.35 (m, 3 H), 1.30-1.42 (m, 4 H), 0.17-0.37 (m, 4 H) |
| Compound | 573.2 | (DMSO-d⁶) δ 8.90-8.96 (m, 1 H), 8.41-8.46 (m, 1 H), 8.09-8.17 |
| 116 | (M + H)⁺ | (m, 1 H), 7.86-7.93 (m, 1 H), 7.63-7.71 (m, 1 H), 7.49-7.55 (m, 1 |
| | | H), 7.16-7.21 (m, 1 H), 7.04-7.11 (m, 1 H), 3.16-3.25 (m, 2 H), |
| | | 2.75-2.82 (m, 4 H), 2.12-2.19 (m, 1 H), 1.31-1.39 (m, 7 H), 1.20- |
| | | 1.26 (m, 9 H), 0.22-0.32 (m, 4 H) |
| Compound | 591.2 | (MeOH-d⁴) δ 8.98-9.10 (m, 1 H) 8.35-8.48 (m, 1 H) 8.07-8.19 (m, |
| 117 | (M + H)⁺ | 1 H) 7.83-7.97 (m, 1 H) 7.62-7.74 (m, 1 H) 7.46-7.57 (m, 2 H) |
| | | 3.09-3.26 (m, 2 H) 2.70-2.85 (m, 4 H) 1.34-1.44 (m, 7 H) 1.12- |
| | | 1.25 (m, 9 H) 0.18-0.33 (m, 4 H) |
| Compound | 597.4 | (DMSO-d⁶) δ 10.05-10.20 (m, 1 H), 9.05-9.11 (m, 1 H), 8.41-8.47 |
| 118 | (M + H)⁺ | (m, 1 H), 8.04-8.11 (m, 1 H), 7.79-7.85 (m, 1 H), 7.60-7.73 (m, 2 |
| | | H), 7.48-7.53 (m, 1 H), 7.08-7.14 (m, 1 H), 6.99 (br d, J = 4.88 Hz, 1 |
| | | H), 5.98 (d, J = 3.75 Hz, 1 H), 5.79-5.88 (m, 1 H), 5.67-5.73 (m, 1 |
| | | H), 3.17-3.24 (m, 2 H), 2.79-2.87 (m, 2 H), 2.55-2.62 (m, 2 H), |
| | | 1.79-1.92 (m, 1 H), 1.56-1.64 (m, 2 H), 1.20-1.29 (m, 5 H), 1.09- |
| | | 1.15 (m, 9 H) |
| Compound | 577.4 | (DMSO-d⁶) δ 9.92-10.08 (1 H, m) 8.44 (1 H, s) 7.44 (1 H, d, J = 8.50 |
| 119 | (M + H)⁺ | Hz) 7.36 (1 H, s) 7.09 (1 H, d, J = 2.25 Hz) 6.98 (1 H, m) 4.87-5.04 |
| | | (1 H, m) 3.72-3.83 (2 H, m) 3.50-3.57 (3 H, m) 3.34 (2 H, br d, J = |
| | | 1.00 Hz) 3.14-3.21 (4 H, m) 2.63-2.71 (4 H, m) 2.06-2.22 (4 H, m) |
| | | 1.26 (4 H, br d, J = 4.63 Hz) 0.17-0.28 (4 H, m) |
| Compound | 591.4 | (DMSO-d⁶) δ 10.09 (br s, 1 H), 9.14 (s, 1 H), 8.39 (m, 1 H), 8.19 (m, 1 |
| 120 | (M + H)⁺ | H), 7.88 (s, 1 H), 7.53-7.61 (m, 1 H), 7.49 (d, J = 8.50 Hz, 1 H), 7.13 |
| | | (d, J = 2.13 Hz, 1 H), 7.02 (m, 1 H), 3.20 (m, 2 H), 2.67 (br t, J = 5.00 |
| | | Hz, 4 H), 1.20-1.30 (m, 7 H), 1.16 (s, 9 H), 0.23 (s, 4 H) |
| Compound | 589.2 | (DMSO-d⁶) δ 9.93-10.16 (m, 1 H) 9.11-9.15 (m, 1 H) 8.43-8.46 |
| 121 | (M + H)⁺ | (m, 1 H) 8.11-8.16 (m, 1 H) 7.79-7.84 (m, 1 H) 7.67-7.73 (m, 1 H) |
| | | 7.61-7.65 (m, 1 H) 7.49-7.53 (m, 1 H) 7.10-7.14 (m, 1 H) 6.99- |
| | | 7.05 (m, 1 H) 4.90-5.05 (m, 1 H) 3.74-3.82 (m, 2 H) 3.34-3.37 (m, |
| | | 2 H) 2.65-2.70 (m, 4 H) 1.22-1.30 (m, 4 H) 1.13 (s, 9 H) 0.19-0.25 |
| | | (m, 4 H) |
| Compound | 615.4 | (DMSO-d⁶) δ 9.15 (s, 1H), 8.45 (s, 1H), 8.14 (d, J = 7.7 Hz, 1H), 7.81 |
| 122 | (M + H)⁺ | (d, J = 8.2 Hz, 1H), 7.66-7.74 (m, 1H), 7.63 (s, 1H), 7.49 (d, J = 8.6 |
| | | Hz, 1H), 7.17 (d, J = 1.9 Hz, 1H), 7.03 (dd, J = 2.1, 8.5 Hz, 1H), 3.78 |
| | | (s, 2H), 2.65 (br t, J = 4.8 Hz, 4H), 1.26 (br s, 4H), 1.12 (s, 11H), 1.02- |
| | | 0.95 (m, 2H), 0.22 (s, 4H) |
| Compound | 526.2 | (DMSO-d⁶) δ 10.05-10.11 (m, 1 H) 8.71-8.75 (m, 1 H) 7.46-7.51 |
| 123 | (M + H)⁺ | (m, 1 H) 7.10-7.13 (m, 1 H) 6.99-7.03 (m, 1 H) 6.76-6.79 (m, 1 H) |
| | | 6.36-6.39 (m, 1 H) 5.33-5.36 (m, 1 H) 4.33-4.37 (m, 1 H) 3.16- |
| | | 3.23 (m, 2 H) 2.64-2.69 (m, 4 H) 1.41-1.83 (m, 9 H) 1.20-1.28 (m, |
| | | 7 H) 0.24 (s, 4 H) |
| Compound | 589.2 | (DMSO-d⁶) δ 9.68-10.35 (m, 1 H), 9.13 (s, 1 H), 8.70 (s, 1 H), 7.59 |
| 124 | (M + H)⁺ | (d, J = 8.8 Hz, 1 H), 7.13 (d, J = 2.0 Hz, 1 H), 7.04 (dd, J = 8.8, 2.0 Hz, |
| | | 1 H), 4.66-5.26 (m, 1 H), 3.78 (t, J = 6.4 Hz, 2 H), 3.45-3.58 (m, 4 |
| | | H), 3.32-3.37 (m, 2 H), 2.65-2.80 (m, 4 H), 2.54 (s, 3 H), 2.07-2.22 |
| | | (m, 4 H), 1.32 (br s, 4 H), 0.24 (s, 4 H) |
| Compound | 510.4 | (CDCl₃) δ 8.46 (s, 1 H) 7.50-7.60 (m, 1 H) 7.14-7.26 (m, 1 H) 6.92- |
| 125 | (M + H)⁺ | 7.11 (m, 2 H) 6.90 (d, J = 3.4 Hz, 1 H) 6.31 (d, J = 3.4 Hz, 1 H) 6.19 (br |
| | | d, J = 2.0 Hz, 1 H) 4.13-4.20 (m, 2 H) 3.29-3.40 (m, 2 H) 2.74-2.90 |
| | | (m, 4 H) 2.62-2.69 (m, 2 H) 2.52-2.60 (m, 2 H) 1.97-2.06 (m, 2 H) |
| | | 1.24-1.48 (m, 5 H) 0.30 (s, 4 H) |
| Compound | 512.4 | (DMSO-d⁶) δ 0.18-0.33 (m, 4 H) 1.20-1.36 (m, 4 H) 1.56-1.80 (m, |
| 126 | (M + H)⁺ | 6 H) 1.92-2.09 (m, 2 H) 2.67 (t, J = 5.0 Hz, 4 H) 3.07-3.22 (m, 1 H) |
| | | 3.32 (br s, 2 H), 3.78 (t, J = 6.4 Hz, 2 H) 6.19-6.32 (m, 1 H) 6.71 (s, 1 |
| | | H) 6.98-7.05 (m, 1 H) 7.09-7.14 (m, 1 H) 7.44 (s, 1 H) 8.68-8.76 |
| | | (m, 1 H) 9.97-10.11 (m, 1 H) |
| Compound | 566.3 | (DMSO-d⁶) δ 9.41 (s, 1 H), 8.07 (d, J = 8.51 Hz, 1 H), 7.14 (d, J = |
| 127A | (M + H)⁺ | 1.88 Hz, 1 H), 7.19 (s, 1 H), 7.03 (dd, J = 8.38, 2.00 Hz, 1 H), 6.51 (s, |
| | | 1 H), 4.95 (br s, 1 H), 4.72 (s, 1 H), 3.75 (t, J = 6.50 Hz, 3 H), 3.83 (d, |
| | | J = 7.00 Hz, 1 H), 3.56 (br d, J = 9.76 Hz, 1 H), 3.21-3.30 (m, 4 H), |
| | | 2.84 (br t, J = 4.63 Hz, 4 H), 2.33-2.39 (m, 3 H), 1.99 (br d, J = 9.76 |
| | | Hz, 1 H), 1.91 (br d, J = 10.01 Hz, 1 H), 1.43-1.70 (m, 4 H), 0.35 (s, |
| | | 4 H). |
| Compound | 566.6 | (DMSO-d⁶) δ 9.73-10.05 (m, 1H), 9.41 (s, 1H), 8.08 (d, J = 8.5 Hz, |
| 127B | (M + H)⁺ | 1H), 7.12-7.22 (m, 2H), 7.00-7.10 (m, 1H), 6.51 (s, 1H), 4.83-5.13 (m, |
| | | 2H), 4.72 (s, 1H), 3.83 (d, J = 7.0 Hz, 1H), 3.70-3.79 (m, 3H), 3.56 (d, |
| | | J = 10.1 Hz, 1H), 3.25-3.29 (m, 3H), 2.84 (br t, J = 4.9 Hz, 4H), 2.36 |
| | | (s, 3H), 1.87-2.04 (m, 2H), 1.47-1.68 (m, 3H), 0.35 (s, 4H) |
| Compound | 536.4 | (DMSO-d⁶) δ 9.93-10.15 (m, 1 H) 8.80-8.94 (m, 1 H) 7.49-7.65 |
| 128 | (M + H)⁺ | (m, 2 H) 7.09-7.16 (m, 1 H) 7.00-7.06 (m, 1 H) 6.91-6.98 (m, 1 H) |
| | | 6.84-6.90 (m, 1 H) 2.99-3.16 (m, 3 H) 2.61-2.83 (m, 4 H) 1.90- |
| | | 2.03 (m, 4 H) 1.64-1.85 (m, 4 H) 1.44-1.61 (m, 2 H) 1.27-1.41 (m, |
| | | 4 H) 0.18-0.38 (m, 4 H) |
| Compound | 527.3 | (DMSO-d⁶) δ 9.85 (br s, 1H), 9.20 (s, 1H), 8.37-8.42 (m, 1H), 8.34- |
| 129 | (M + H)⁺ | 8.37 (m, 1H), 7.85-7.95 (m, 2H), 7.14 (d, J = 2.0 Hz, 1H), 7.02 (dd, J = |
| | | 8.4, 2.0 Hz, 1H), 4.71-5.20 (m, 1H), 4.51 (t, J = 12.4 Hz, 4H), 3.78- |
| | | 3.74 (m, 2H), 3.26-3.31 (m, 2H), 2.93-2.72 (m, 4H), 1.48 (br s, 4H), |
| | | 0.32 (s, 4H) |
| Compound | 576/4 | (DMSO-d⁶) δ 9.98 (br s, 1 H) 8.65 (s, 1 H) 7.90 (d, J = 2.13 Hz, 1 H) |
| 130 | (M + H)⁺ | 7.45 (d, J = 8.50 Hz, 1 H) 7.05 (d, J = 1.88 Hz, 1 H) 6.98 (dd, J = 8.50, |
| | | 1.88 Hz, 1 H) 6.67 (d, J = 2.13 Hz, 1 H) 4.99 (br s, 1 H) 4.41-4.53 |
| | | (m, 1 H) 3.77 (t, J = 6.57 Hz, 2 H) 3.34 (br s, 2 H) 2.53 (br d, J = 4.25 |
| | | Hz, 4 H) 1.98-2.22 (m, 8 H) 1.75-1.85 (m, 2 H) 1, 63-1.71 (m, 4 H) |
| | | 1.42-1.52 (m, 4 H) |
| Compound | 584.4 | (DMSO-d⁶) δ 10.07-10.10 (m, 1 H), 9.01-9.05 (m, 1 H) 7.59 (d, |
| 131 | (M + H)⁺ | J = 8.63 Hz, 1 H), 7.39 (s, 1 H), 7.11-7.15 (m, 1 H), 7.03 (dd, J = 8.50, |
| | | 2.25 Hz, 1 H), 6.85-6.89 (m, 1 H), 4.54 (s, 2 H), 3.75-3.83 (m, 5 H), |
| | | 3.16-3.24 (m, 2 H), 2.65-2.69 (m, 4 H), 1.93-2.07 (m, 4 H), 1.31 |
| | | (br s, 4H), 1.21 (s, 1 H), 1.19-1.26 (m, 1 H), 0.24 (s, 4 H) |
| Compound | 563.5 | (DMSO-d⁶) δ 8.90 (s, 1 H) 7.95 (s, 1 H) 7.56-7.58 (m, 1 H) 7.09- |
| 132 | (M + H)⁺ | 7.22 (m, 2 H) 6.99-7.07 (m, 2 H) 3.15-3.23 (m, 2 H) 2.65 (br t, J = |
| | | 5.07 Hz, 4 H)1.25 (s, 4 H) 1.23 (s, 3 H) 1.19 (s, 9 H) 0.23 (s, 4 H) |
| Compound | 537.4 | (DMSO-d⁶) δ 10.05-10.13 (br s, 1 H) 8.99 (s, 1 H) 7.76 (s, 1 H) 7.56 |
| 133 | (M + H)⁺ | (d, J = 8.40 Hz, 1 H) 7.29 (s, 1 H) 7.12 (d, J = 2.40 Hz, 1 H) 7.02-7.04 |
| | | (m, 1 H) 5.31 (d, J = 5.20 Hz, 1 H) 4.48-4.51 (m, 1 H) 3.19-3.28 (m, |
| | | 2 H) 2.63-2.69 (m, 5 H) 2.40 (s, 3 H) 1.91-2.13 (m, 2 H) 1.76-1.79 |
| | | (m, 4 H) 1.20-1.28 (m, 7 H) 0.22 (s, 4 H) |
| Compound | 555.3 | (DMSO-d⁶) δ 10.09 (s, 1 H), 9.09 (s, 1 H), 7.99-8.03 (d, J = 4.00 Hz, 1 |
| 134 | (M + H)⁺ | H), 7.76-7.80 (m, 1 H), 7.54-7.56 (m, 1 H), 7.12 (m, 1 H), 7.01- |
| | | 7.04 (m, 1 H), 5.26-5.27 (m, 1 H), 4.61-4.66 (m, 1 H), 3.18-3.20 |
| | | (m, 2 H), 2.62-2.71 (m, 5 H), 1.54-1.66 (m, 7 H), 1.20-1.28 (m, 6 |
| | | H), 0.21-0.24 (s, 4 H) |
| Compound | 551.3 | (DMSO-d⁶) δ 9.94 (br s, 1H), 9.02 (s, 1H), 7.34 (d, J = 8.5 Hz, 1H), |
| 135 | (M + H)⁺ | 6.94 (d, J = 2.3 Hz, 1H), 6.85 (dd, J = 2.3, 8.6 Hz, 1H), 3.66 (br t, J = |
| | | 5.6 Hz, 4H), 2.93 (s, 3H), 2.32 (td, J = 1.8, 3.5 Hz, 4H), 1.84-1.97 |
| | | (m, 4H), 1.05 (br s, 4H), 0.06 (s, 4H) |
| Compound | 555.9 | (DMSO-d⁶) δ 8.98 (s, 1 H), 7.57-7.61 (m, 2 H), 7.11-7.13 (m, 1 H), |
| 136 | (M + H)⁺ | 7.02-7.04 (m, 1 H), 6.88 (m, 1 H), 3.76-3.79 (m, 2 H), 3.55-3.56 |
| | | (m, 4 H), 3.35 (br s, 1 H), 3.30 (m, 1 H), 2.65-2.68 (m, 4 H), 1.59 (m, |
| | | 6 H), 1.32 (m, 4 H), 0.25 (s, 4 H) |
| Compound | 558.9 | (DMSO-d⁶) δ 10.09 (br s, 1 H), 9.15 (s, 1 H), 8.51 (s, 1 H), 8.43 (m, 1 |
| 137 | (M + H)⁺ | H), 7.55-7.58 (m, 1 H), 7.13 (m, 1 H), 7.02-7.05 (m, 1 H), 3.84- |
| | | 3.87 (m, 4 H), 3.19-3.32 (m, 2 H), 2.68-2.68 (m, 4 H), 2.01-2.07 |
| | | (m, 4 H), 120-1.29 (m, 7 H), 0.24 (s, 4 H) |
| Compound | 551.3 | (DMSO-d⁶) δ 10.11 (br s, 1 H) 8.98 (s, 1 H) 7.77 (br s, 1 H) 7.56 (d, |
| 138 | (M + H)⁺ | J = 8.4 Hz, 1 H) 7.27 (br s, 1 H) 7.13 (d, J = 2.4 Hz, 1 H) 7.02 (dd, J = 2.0 |
| | | Hz, 2.4 Hz 1 H) 5.28 (d, J = 5.2 Hz, 1 H) 4.39-4.48 (m, 1 H) 3.16- |
| | | 3.25 (m, 2 H) 2.58-2.73 (m, 4 H) 2.41 (s, 3 H) 2.24-2.35 (m, 1 H) |
| | | 1.40-1.60 (m, 8 H) 1.18-1.33 (m, 7 H) 0.21 (br s, 4 H) |
| Compound | 563.3 | (DMSO-d⁶) δ 10.03 (br s, 1 H) 9.00 (s, 1 H) 7.77 (s, 1 H) 7.56 (d, |
| 139 | (M + H)⁺ | J = 8.8 Hz, 1 H) 7.23-7.31 (m, 1 H) 7.15 (d, J = 2.4 Hz, 1 H) 7.05 (dd, |
| | | J = 2.0 Hz, 2.0 Hz 1 H) 5.29 (d, J = 4.8 Hz, 1 H) 4.37-4.49 (m, 1 H) 2.59- |
| | | 2.79 (m, 5 H) 2.41 (s, 3 H) 2.24-2.36 (m, 1 H) 1.39-1.60 (m, 8 H) |
| | | 1.20-1.35 (m, 4 H) 1.00 (d, J = 6.4 Hz, 4 H) 0.23 (s, 4 H) |
| Compound | 592.4 | (DMSO-d⁶) δ 9.64-9.98 (m, 1H) 9.30-9.35 (m, 1H) 8.02-8.09 (m, |
| 140 | (M + H)⁺ | 1H) 7.80-7.89 (m, 1H) 7.54-7.61 (m, 1H) 7.13-7.18 (m, 1H) 7.02- |
| | | 7.08 (m, 1H) 4.80-5.04 (m, 1H) 3.71-3.79 (m, 6H) 3.25-3.30 (m, |
| | | 2H) 2.80-2.88 (m, 4H) 2.10-2.22 (m, 4H) 1.33-1.73 (m, 4H) 0.33- |
| | | 0.40 (m, 4H) |
| Compound | 580.4 | (DMSO-d⁶) δ 9.70-9.97 (m, 1 H) 9.07-9.11 (m, 1 H) 8.02-8.08 (m, |
| 141 | (M + H)⁺ | 1 H) 7.82-7.90 (m, 1 H) 7.55-7.61 (m, 1 H) 7.11-7.15 (m, 1 H) |
| | | 7.01-7.06 (m, 1 H) 4.78-5.06 (m, 1 H) 3.67-3.78 (m, 6 H) 3.26- |
| | | 3.28 (m, 2 H) 2.97-3.05 (m, 2 H) 2.57-2.62 (m, 2 H) 2.09-2.20 (m, |
| | | 4 H) 1.71-1.79 (m, 2 H) 1.45-1.59 (m, 1 H) 1.23-1.37 (m, 2 H) |
| | | 0.97-0.97 (m, 2 H) 0.93-1.01 (m, 3 H) |
| Compound | 554.4 | (DMSO-d⁶) δ 9.87 (s, 1 H), 9.37 (s, 1 H), 8.05-8.09 (m, 1 H), 7.64- |
| 142 | (M + H)⁺ | 7.72 (m, 1 H), 7.25-7.29 (m, 1 H), 7.17 (d, J = 1.96 Hz, 1 H), 7.03- |
| | | 7.07 (m, 1 H), 3.92-3.95 (dd, J = 10.45, 3.36 Hz, 2 H), 3.75 (m, 2 H), |
| | | 3.69 (m, 2 H), 3.29 (m, 2 H), 2.85 (m, 4 H), 1.68-1.74 (m, 2 H), 1.46- |
| | | 1.68 (m, 4 H), 0.74-0.81 (m, 1 H), 0.39 (s, 4 H), 0.28-0.31 (m, 1 |
| | | H) |
| Compound | 563.4 | (DMSO-d⁶) δ 9.02-9.09 (m, 1 H), 8.50-8.57 (m, 1 H), 8.41-8.46 |
| 143 | (M + H)⁺ | (m, 1 H), 7.50-7.57 (m, 1 H), 7.06-7.12 (m, 1 H), 6.99-7.04 (m, 1 |
| | | H), 4.76-5.17 (m, 1 H), 3.81-3.88 (m, 4 H), 3.74-3.80 (m, 2 H), |
| | | 3.33-3.36 (m, 1 H), 3.29-3.31 (m, 1 H), 2.69-2.79 (m, 2 H), 2.52- |
| | | 2.56 (m, 2 H), 1.98-2.12 (m, 4 H), 1.50-1.60 (m, 2 H), 1.31-1.45 |
| | | (m, 1 H), 0.97-1.14 (m, 2 H), 0.78-0.87 (m, 3 H) |
| Compound | 593.4 | (DMSO-d⁶) δ 9.88 (1 H, br d, J = 2.00 Hz) 9.27 (1 H, s) 8.52 (1 H, s) |
| 144 | (M + H)⁺ | 8.45 (1 H, s) 7.68 (1 H, d, J = 10.38 Hz) 7.35 (1 H, d, J = 7.75 Hz) |
| | | 5.02 (1 H, br d, J = 2.88 Hz) 3.84-3.89 (4 H, m) 3.79-3.84 (2 H, m) |
| | | 3.36 (2 H, br s) 2.68 (2 H, br s) 1.96-2.12 (4 H, m) 1.31 (4 H, br s) |
| | | 0.25 (4 H, s) |
| Compound | 573.4 | (DMSO-d⁶) δ 10.05 (br s, 1 H) 9.13 (s, 1H), 8.70 (s, 1H) 7.58 (d, |
| 145 | (M + H)⁺ | J = 8.00 Hz, 1H) 7.13 (d, J = 1.88 Hz, 1 H) 7.02-7.06 (m, 1 H) 3.45- |
| | | 3.49 (m, 4 H) 3.14-3.26 (m, 2 H) 2.66-2.69 (m, 4 H) 2.54 (br s, 3 H) |
| | | 2.09-2.21 (m, 4 H) 1.29-1.38 (m, 4 H) 1.20-1.26 (m, 3 H) 0.20- |
| | | 0.28 (m, 4 H) |
| Compound | 575.2 | (DMSO-d⁶) δ 9.76-10.29 (m, 1 H) 9.14-9.28 (m, 1 H) 8.16-8.23 |
| 146 | (M + H)⁺ | (m, 1 H) 8.05-8.13 (m, 1 H) 7.48-7.59 (m, 1 H) 7.09-7.17 (m, 1 H) |
| | | 6.97-7.05 (m, 1 H) 3.73-3.91 (m, 4 H) 3.13-3.23 (m, 2 H) 2.63- |
| | | 2.68 (m, 4 H) 1.96-2.10 (m, 4 H) 1.25-1.44 (m, 4 H) 1.01-1.23 (m, |
| | | 3 H) 0.12-0.37 (m, 4 H) |
| Compound | 555.2 | (DMSO-d⁶) δ 9.85 (br s, 1 H), 9.34 (s, 1 H), 8.08 (d, J = 8.41 Hz, 1 H), |
| 147 | (M + H)⁺ | 7.76 (dd, J = 12.92, 8.28 Hz, 1 H), 7.46 (dd, J = 8.28, 1.76 Hz, 1 H) 7.16 |
| | | (d, J = 1.76 Hz, 1 H), 7.04-7.06 (dd, J = 8.28, 1.63 Hz, 1 H) 4.93 (m, 1 |
| | | H), 3.73-3.76 (br t, J = 6.46 Hz, 2 H), 3.59 (m, 4 H), 3.27 (br s, 2 H), |
| | | 2.84 (m, 4 H), 1.37-1.79 (m, 10 H), 0.35 (s, 4 H). |
| Compound | 576.4 | (DMSO-d⁶) δ 10.09 (s, 1H), 9.04 (s, 1H), 7.66 (dd, J = 8.1, 13.3 Hz, |
| 148 | (M + H)⁺ | 1H), 7.58 (d, J = 8.5 Hz, 1H), 7.53 (dd, J = 2.8, 8.1 Hz, 1H), 7.13 (d, J = |
| | | 2.3 Hz, 1H), 7.04 (dd, J = 2.3, 8.6 Hz, 1H), 3.65-3.74 (m, 4H), 3.20 |
| | | (q, J = 7.4 Hz, 2H), 2.68 (br t, J = 4.9 Hz, 4H), 2.02-2.16 (m, 4H), |
| | | 1.32 (br s, 4H), 1.23 (t, J = 7.3 Hz, 3H), 0.24 (s, 4H) |
| Compound | 562.4 | (DMSO-d⁶) δ 10.05 (s, 1H), 9.04 (s, 1H), 7.64-7.67 (dd, J = 8.1, 13.3 |
| 149 | (M + H)⁺ | Hz, 1H), 7.58-7.60 (d, J = 8.5 Hz, 1H), 7.54 (dd, J = 2.7, 8.1 Hz, 1H), |
| | | 7.11 (d, J = 2.3 Hz, 1H), 7.02-7.04 (dd, J = 2.3, 8.6 Hz, 1H), 3.68- |
| | | 3.70 (m, 4H), 3.09 (s, 3H), 2.67-2.70 (m, 4H), 2.08-2.11 (m, 4H), |
| | | 1.33 (br s, 4H), 0.25 (s, 4H) |
| Compound | 540.4 | (DMSO-d⁶) δ 9.86-10.05 (m, 1 H), 9.21 (s, 1 H), 8.79 (s, 1 H), 8.22 |
| 150 | (M + H)⁺ | (m, 1 H), 7.60-7.62 (m, 1 H), 7.13 (m, 1 H), 7.03-7.05 (m, 1 H), |
| | | 5.43-5.46 (m, 1 H), 4.76-5.16 (m, 1 H), 3.76-3.96 (m, 2 H), 3.34- |
| | | 3.37 (m, 2 H), 2.66-2.69 (m, 4 H), 2.06-2.09 (m, 2 H), 1.59-1.79 |
| | | (m, 6 H), 1.32 (m, 4 H), 0.23 (s, 4 H) |
| Compound | 603.4 | (DMSO-d⁶) δ 10.12 (br s, 1 H) 9.22 (s, 1 H) 7.58 (d, J = 8.80 Hz, 1 H) |
| 151 | (M + H)⁺ | 7.21 (s, 1 H) 7.12 (m, 1 H) 7.02-7.04 (m, 1 H) 4.66 (br s, 1 H) 3.96- |
| | | 3.99 (m, 4 H) 3.426 (m, 2 H) 3.19-3.21 (m, 2 H) 2.65-2.67 (m, 4 H) |
| | | 2.40 (s, 3 H) 2.07 (s, 1 H) 1.99-2.07 (m, 4 H) 1.83 (m, 2 H) 1.28 (m, |
| | | 4 H) 0.24 (s, 4 H) |
| Compound | 575.4 | (DMSO-d⁶) δ 10.02 (br s, 1H), 9.20 (s, 1H), 7.89 (s, 1H), 7.55-7.57 |
| 152 | (M + H)⁺ | (m, 1H), 7.12 (m, 1H), 7.02-7.04 (m, 1H), 4.98 (m, 1H), 3.94 (m, |
| | | 2H), 3.81-3.90 (m, 4H), 3.76-3.81 (m, 2H), 3.35 (m, 3H), 2.67 (m, |
| | | 4H), 2.54 (s, 3H), 1.23-1.30 (m, 4H), 0.23 (s, 4H) |
| Compound | 554.4 | (DMSO-d⁶) δ 10.01-10.04 (br s, 1 H) 8.97 (s, 1 H) 7.60 (d, J = 8.4 Hz, 1 |
| 153 | (M + H)⁺ | H) 7.45-7.55 (m, 1 H) 7.26 (dd, J = 2.4 Hz, 2.4 Hz, 1 H) 7.12 (d, J = 2.4 |
| | | Hz, 1 H) 7.01-7.04 (m, 1 H) 4.96-5.01 (br s, 1 H) 3.90-3.93 (dd, |
| | | J = 10.48, 3.20 Hz, 1 H) 3.75-3.77 (m, 2 H) 3.60 (m, 2 H) 3.34 (m, 2 H) |
| | | 2.67-2.70 (m, 4 H) 1.65-1.67 (m, 2 H) 1.23-1.35 (m, 4 H) 0.69- |
| | | 0.72 (m, 1 H) 0.27 (s, 5 H) |
| Compound | 558.4 | (DMSO-d⁶) δ 9.87-10.19 (br s, 1H), 9.05 (s, 1H), 7.70-7.74 (m, 2H), |
| 154 | (M + H)⁺ | 7.58-7.59 (d, J = 8.7 Hz, 1H), 7.13 (d, J = 2.1 Hz, 1H), 7.03 (dd, J = |
| | | 2.0, 8.4 Hz, 1H), 4.97 (br s, 1H), 4.06 (t, J = 5.1 Hz, 2H), 3.78 (t, J = |
| | | 6.6 Hz, 2H), 3.68 (m, 2H), 3.35 (m, 2H), 2.66-2.69 (br t, J = 5.0 Hz, |
| | | 4H), 1.86 (m, 2H), 1.72 (m, 2H), 1.33 (m, 4H), 0.25 (s, 4H) |
| Compound | 586.4 | (DMSO-d⁶) δ 10.10 (br s, 1H), 9.30 (s, 1H), 7.82 (s, 1H), 7.59 (d, J = |
| 155 | (M + H)⁺ | 8.6 Hz, 1H), 7.19 (br s, 1H), 7.14 (d, J = 2.0 Hz, 1H), 7.03 (dd, J = 2.1, |
| | | 8.6 Hz, 1H), 4.72 (s, 1H), 3.76-3.79 (t, J = 6.5 Hz, 2H), 3.27 (m, 2H), |
| | | 2.83-2.91 (m, 4H), 2.66-2.69 (br t, J = 4.9 Hz, 4H), 2.56 (s, 3H), |
| | | 2.19-2.21 (td, J = 7.0, 13.6 Hz, 2H), 1.30 (m, 4H), 0.25 (s, 4H) |
| Compound | 559.5 | (DMSO-d⁶) δ 9.13 (s, 1H) 8.70 (s, 1H) 7.60 (d, J = 8.40 Hz, 1H) 7.02- |
| 156 | (M + H)⁺ | 7.12 (m, 2 H) 3.44-3.47 (m, 4 H) 3.09 (s, 3 H) 2.67-2.70 (m, 3 H) |
| | | 2.54 (s, 3 H) 2.14-2.15 (m, 4 H) 1.31 (m, 4 H) 0.24 (s, 4 H) |
| Compound | 592.3 | (DMSO-d⁶) δ 9.96 (br s, 1 H) 8.98 (s, 1 H) 7.64-7.67 (m, 1 H) 7.51- |
| 157 | (M + H)⁺ | 7.56 (m, 2 H) 7.13 (d, J = 1.75 Hz, 1 H) 7.02-7.04 (m, 1 H) 4.99 (m, 1 |
| | | H) 3.78 (s, 2 H) 3.68 (br t, J = 5.50 Hz, 4 H) 2.59 (br d, J = 5.38 Hz, 4 |
| | | H) 2.07-2.12 (m, 4 H) 1.42-1.47 (m, 6 H) 1.10-1.12 (m, 2 H) 0.98- |
| | | 1.00 (m, 2 H) |
| Compound | 619.3 | (DMSO-d⁶) δ 9.30 (s, 1 H) 7.58-7.64 (m, 2 H) 7.14 (d, J = 1.63 Hz, 1 |
| 158 | (M + H)⁺ | H) 7.03-7.06 (m, 1 H) 4.99 (br s, 1 H) 4.03 (br s, 4H) 3.77-3.80 (m, 2 |
| | | H) 3.35-3.37 (m, 2 H) 2.68-2.71 (m, 4 H) 1.95-2.09 (m, 4 H) 1.24- |
| | | 1.34 (m, 4 H) 0.21-0.28 (m, 4 H) |
| Compound | 589.3 | (DMSO-d⁶) δ 9.90-10.20 (m, 1 H), 9.11-9.14 (m, 1 H), 7.84-7.91 |
| 159 | (M + H)⁺ | (m, 1 H), 7.70-7.74 (m, 1 H), 7.58-7.62 (m, 1 H), 7.14 (d, J = 2.00 Hz, |
| | | 1 H), 7.02-7.08 (m, 1 H), 6.62-6.68 (m, 1 H), 4.78-5.12 (m, 1 H), |
| | | 3.75-3.81 (m, 2 H), 3.33-3.39 (m, 2 H), 2.76-2.90 (m, 4 H), 2.69 (br |
| | | t, J = 4.82 Hz, 4 H), 2.14-2.27 (m, 2 H), 1.34 (br s, 4 H), 0.22-0.30 (m, |
| | | 4 H) |
| Compound | 605.4 | (DMSO-d⁶) δ 10.04 (br s, 1 H) 9.24 (s, 1 H) 7.58 (d, J = 8.40 Hz, 1 H) |
| 160 | (M + H)⁺ | 7.46 (s, 1 H) 7.04 (s, 1 H) 7.02 (d, J = 1.60 Hz, 1 H) 5.55-5.58 (m, 1 |
| | | H) 4.97 (br s, 1 H) 4.46 (d, J = 6.00 Hz, 1 H) 3.97 (s, 4 H) 3.76-3.79 |
| | | (m, 2 H) 3.35-3.37 (m, 2 H,) 2.66-2.67 (m, 4 H) 1.96-2.03 (m, 4 H) |
| | | 1.34 (s, 4 H) 0.24 (s, 4 H) |
| Compound | 536.4 | (DMSO-d⁶) δ 9.93-10.15 (m, 1 H) 9.13-9.17 (m, 1 H) 8.94-8.97 (m, |
| 161 | (M + H)⁺ | 1 H) 7.57-7.62 (m, 1 H) 7.11-7.14 (m, 1 H) 7.01-7.06 (m, 1 H) 6.50- |
| | | 6.53 (m, 1 H) 4.91-5.07 (m, 1 H) 3.75-3.80 (m, 2 H) 2.91-2.97 (m, |
| | | 2 H) 2.62-2.72 (m, 11 H) 1.91-1.99 (m, 2 H) 1.28-1.36 (m, 4 H) |
| | | 0.24-0.25 (m, 4 H) |
| Compound | 589.3 | (DMSO-d⁶) δ 10.16 (s, 1 H), 9.11-9.13 (m, 1 H), 7.96-8.00 (m, 1 H), |
| 162 | (M + H)⁺ | 7.86 (dd, J = 11.55, 8.50 Hz, 1 H), 7.57-7.61 (m, 1 H), 7.11-7.14 (m, 1 |
| | | H), 7.01-7.06 (m, 1 H), 6.51-6.57 (m, 1 H), 4.88-5.06 (m, 1 H), 4.88- |
| | | 5.06 (m, 1 H), 3.75-3.81 (m, 2 H), 3.35-3.37 (m, 2 H), 2.80-2.94 |
| | | (m, 4 H), 2.64-2.73 (m, 4 H), 2.14-2.27 (m, 2 H), 1.29-1.37 (m, 4 |
| | | H), 0.23-0.27 (m, 4 H) |
| Compound | 519.3 | (DMSO-d⁶) δ 10.30 (s, 1H), 8.01-7.96 (m, 1H), 7.81-7.74 (m, 1H), |
| 163 | (M + H)⁺ | 7.47-7.43 (m, 1H), 7.10-7.03 (m, 2H), 7.00-6.95 (m, 1H), 3.77- |
| | | 3.69 (m, 4H), 3.60-3.53 (m, 4H), 3.14 (s, 3H), 3.01-2.93 (m, 4H), |
| | | 1.61-1.46 (m, 4H), 0.36-0.29 (m, 4H) |
| Compound | 511.1 | (DMSO-d⁶) δ 10.55 (br s, 1H), 7.85-7.93 (m, 2H), 7.77 (br d, J = 8.0 |
| 164 | (M + H)⁺ | Hz, 1H) 7.66-7.73 (m, 1H), 7.34 (br d, J = 8.4 Hz, 1H), 6.99 (d, J = 8.4 |
| | | Hz, 1H), 4.08-4.21 (m, 2H), 3.86-3.99 (m, 4H), 3.59-3.67 (m, 4H), |
| | | 3.23 (br d, J = 11.6 Hz, 2H), 3.10 (s, 3H) 2.94-3.05 (m, 2H), 1.18-1.23 |
| | | (m, 2H), 0.60-0.75 (m, 4 H) |

### Biological Assays

### Inhibition of KIF18A Microtubule-Dependent ATPase Activity

Test compounds were plated in a 3× dilution scheme in a 384-well plate. Assay buffer: 80 mM PIPES (pH 6.9), 1 mM MgCl₂, 75 mM KCl, 1 mM EGTA, 1 mM DTT, 0.01% BSA, 0.005% Tween-20, 1 μM Taxol in H2O. To 50 nL of compound in DMSO was added 2.5 μL of enzyme mix [4 nM hKIF18A (1-374) in assay buffer]. After incubation at room temperature for 30 min, 2.5 μL of microtubule mix was added [0.2 mg/mL pre-formed microtubules, 2.0 mM ATP in assay buffer], the plate was centrifuged for 30 s and then incubated at 28° C. for 60 min. 5 μL of Promega® ADP-Glo Max R1 was added, the plate was centrifuged for 30 s, and the mixture incubated for 4 h at room temperature. 10 μL of Promega® ADP-Glo Max R2 was added, the plate centrifuged for 30 s, and incubated for 60 min at room temperature. Luminescence was measured with an Envision plate reader, and % Inhibition was calculated for each well as: ([max−min]−[test−min])/[max−min]. IC50 values were calculated from concentration vs. % Inhibition data via a four-parameter variable slope model. Results from the biological assay are summarized in Table 8.

Table 8 indicates that compounds as provided herein are potent inhibitors of KIF18a. As a comparison, the data for AMG650 (2-(6-azaspiro[2.5]octan-6-yl)-N-[2-(4,4-difluoropiperidin-1-yl)-6-methylpyrimidin-4-yl]-4-(2-hydroxyethanesulfonamido)benzamide) is 17 nM.

### Binding Kinetics to KIF18a-Microtubule Complex

Compound binding kinetics parameters (kₒₙ and k_{off}) were determined by the method of global progress curve analysis (GPCA). KIF18A (0.25 nM) was incubated for up to 24 hr with serially diluted compound in the assay buffer containing 80 mM PIPES, pH 6.9, 1 mM ATP, 0.1 mg/ml preformed microtubule from porcine brain (Cytoskeleton), 1 mM MgCl₂, 1 μM Taxol, 75 mM KCl, 1 mM EGTA, 1 mM DTT, 0.01% BSA and 0.005% Tween-20. ADP product levels were determined by the Promega® ADP-Glo assay. The time/dose-dependent progress curves were then globally fit to a Michaelis-Menten kinetics model with 1-step slow binding inhibition to derive both on-rate kₒₙ and off-rate k_{off} values (Zhang, R., Wong, K. (2017): “High performance enzyme kinetics of turnover, activation and inhibition for translational drug discovery”, Expert Opinion on Drug Discovery, 2017 January; 12(1):17-37. doi: 10.1080/17460441.2017.1245721).

Results from the binding kinetics assay are summarized in Table 9. The data in Table 9 indicate that compounds as provided herein can achieve sub-nanomolar potency with small off-rates, or very long dissociation half-life (ln(2)/k_{off}). As a comparison, the data for AMG650 (2-(6-azaspiro[2.5]octan-6-yl)-N-[2-(4,4-difluoropiperidin-1-yl)-6-methylpyrimidin-4-yl]-4-(2-hydroxyethanesulfonamido)benzamide) are: kₒₙ=0.059 nM⁻¹ h⁻¹; k_{off}=0.21 h⁻¹, dissociation t_{1/2}=4.1 h; K₁=3.4 nM.

### Cell Viability of KIF18a-Sensitive Cell Lines

Cell lines were seeded as follows 24 hours before compound treatment: HCC15 (Korean Cell Line Bank) 600 cell/well, 95 μL of RPMI-1640 media supplemented with 100 units/mL penicillin, 100 units/mL streptomycin and 10% FBS; NIH:OVCAR-3 (ATCC), 1000 cell/well, 95 μL of RPMI-1640 media supplemented with 100 units/mL penicillin, 100 units/mL streptomycin, 0.01 mg/mL bovine insulin, and 20% FBS; JIMT-1 (Addexbio) 1000 cell/well, 95 μL of DMEM media supplemented with 100 units/mL penicillin, 100 units/mL streptomycin, and 10% FBS.

Test compounds were added to cells in a 20× dilution scheme by adding 5 μL of serially diluted compound to the plate, and the treated cells were incubated for an additional 7 days in a 37° C., 5% CO₂ incubator. DMSO was used as the negative control (0% effect), and wells omitting cells were used as the positive control (100% effect). The cells were incubated for seven days, and cell viability determined via the Promega Cell Titre-Glo® Assay kit. Luminescence units were converted to ATP concentrations via an ATP standard curve (10 point, 2-fold dilution from 5 uM). % Inhibition was calculated for each well as: ([max−min]−[test−min])/[max−min]. IC₅₀ values were calculated from concentration vs. % Inhibition data via a four-parameter variable slope model. Results from the biological assay are summarized in Table 10.

Table 10 indicates that compounds as provided herein potently inhibit cell growth or induce cell killing for KIF8a-sensitive cancer cell lines. As a comparison, the data for AMG650 (2-{6-azaspiro[2.5]octan-6-yl}-N-[2-(4,4-difluoropiperidin-1-yl)-6-methylpyrimidin-4-yl]-4-(2-hydroxyethanesulfonamido)benzamide) are: HCC-15, 0.066 μM; JIMT-10.13 μM; NIH: OVCAR30.10 μM.

TABLE 8
Summary of biochemical assay data

| Compound | IC₅₀ (nM) |
|---|---|
| Compound 1 | 1,200 |
| Compound 2 | 14 |
| Compound 3 | 280 |
| Compound 4 | 5,600 |
| Compound S | 6.6 |
| Compound 6 | 25 |
| Compound 7 | 83 |
| Compound 8 | 5.1 |
| Compound 9 | 21 |
| Compound 10 | 9.2 |
| Compound 11 | 23 |
| Compound 12 | 9.1 |
| Compound 13 | 7.2 |
| Compound 14 | 39 |
| Compound 15 | 6.6 |
| Compound 16 | 1100 |
| Compound 17 | 29 |
| Compound 18 | 8.7 |
| Compound 19 | 14 |
| Compound 20 | 23 |
| Compound 21 | 32 |
| Compound 22 | 11 |
| Compound 23 | 11 |
| Compound 25 | 4.7 |
| Compound 26 | 44 |
| Compound 27 | >3.00E+03 |
| Compound 28 | 45 |
| Compound 29 | 18 |
| Compound 30 | 21 |
| Compound 32 | 140 |
| Compound 33 | 36 |
| Compound 34 | 74 |
| Compound 35 | 27 |
| Compound 41 | 16 |
| Compound 45 | 6.6 |
| Compound 46 | 9.1 |
| Compound 47 | 19 |
| Compound 48 | 17 |
| Compound 49 | 19 |
| Compound 50 | 7.2 |
| Compound 51 | 2.8 |
| Compound 51a | 2.4 |
| Compound 51b | 3.1 |
| Compound 52 | 18 |
| Compound 53 | 43 |
| Compound 54 | 8.3 |
| Compound 55 | 29 |
| Compound 56 | 13 |
| Compound 57 | 37 |
| Compound 58 | 41 |
| Compound 59 | 1.6 |
| Compound 60 | 6.5 |
| Compound 61 | 8.0 |
| Compound 62 | 18 |
| Compound 63 | 3.4 |
| Compound 64 | 25 |
| Compound 65 | 8.6 |
| Compound 66 | 14 |
| Compound 67 | 22 |
| Compound 68 | 12 |
| Compound 69 | 6.0 |
| Compound 70 | 27 |
| Compound 71 | 10 |
| Compound 73 | 11 |
| Compound 74 | 18 |
| Compound 75 | 7.1 |
| Compound 76 | 29 |
| Compound 77 | 12 |
| Compound 78 | 17 |
| Compound 79 | 15 |
| Compound 80 | 13 |
| Compound 81 | 140 |
| Compound 82 | 60 |
| Compound 83 | 10 |
| Compound 84 | 8.2 |
| Compound 85 | 11 |
| Compound 86 | 27 |
| Compound 87 | 27 |
| Compound 88 | 120 |
| Compound 89 | 21 |
| Compound 90a | 5.2 |
| Compound 90b | 5.6 |
| Compound 91 | 36 |
| Compound 92 | 9.6 |
| Compound 93 | 10 |
| Compound 94 | 12 |
| Compound 95 | 38 |
| Compound 96 | 4.4 |
| Compound 97 | 36 |
| Compound 98 | 12 |
| Compound 99 | 8.1 |
| Compound 100 | 11 |
| Compound 101 | 17 |
| Compound 102 | 16 |
| Compound 103 | 20 |
| Compound 104 | 15 |
| Compound 105 | 75 |
| Compound 106 | 32 |
| Compound 107 | 16 |
| Compound 108 | 63 |
| Compound 109 | 8.8 |
| Compound 110 | 15 |
| Compound 111 | 68 |
| Compound 112 | 77 |
| Compound 113 | 23 |
| Compound 114 | 58 |
| Compound 115 | 44 |
| Compound 116 | 14 |
| Compound 117 | 20 |
| Compound 118 | 15 |
| Compound 119 | 42 |
| Compound 120 | 19 |
| Compound 121 | 14 |
| Compound 122 | 18 |
| Compound 123 | 4.6 |
| Compound 124 | 7.3 |
| Compound 125 | 9.0 |
| Compound 126 | 71 |
| Compound 127A | 11 |
| Compound 127B | 43 |
| Compound 128 | 98 |
| Compound 129 | 14 |
| Compound 130 | 5.0 |
| Compound 131 | 29 |
| Compound 132 | 8.5 |
| Compound 133 | 12 |
| Compound 134 | 14 |
| Compound 135 | 150 |
| Compound 136 | 6.5 |
| Compound 137 | 6.3 |
| Compound 138 | 26 |
| Compound 139 | 40 |
| Compound 140 | 18 |
| Compound 141 | 17 |
| Compound 142 | 26 |
| Compound 143 | 5.5 |
| Compound 144 | 2.8 |
| Compound 145 | 17 |
| Compound 146 | 4.4 |
| Compound 147 | 22 |
| Compound 148 | 31 |
| Compound 149 | 21 |
| Compound 150 | 6.0 |
| Compound 151 | 27 |
| Compound 152 | 14 |
| Compound 153 | 16 |
| Compound 154 | 7.1 |
| Compound 157 | 21 |
| Compound 158 | 3.8 |
| Compound 159 | 14 |
| Compound 160 | 4.4 |
| Compound 161 | 63 |
| Compound 162 | 20 |
| Compound 163 | 3600 |
| Compound 164 | >30,000 |
| Compound 165 | 283 |
| Compound 166 | 2800 |

TABLE 9
Summary of kinetic assay data

| Compound | kₒₙ (nM⁻¹h⁻¹)^{a} | k_{off} (h⁻¹)^{b} | disc. t_{1/2} (h)^{c} | K_{I} (nM)^{d} |
|---|---|---|---|---|
| Compound 2 | 0.014 | 0.13 | 5.5 | 8.9 |
| Compound 5 | 0.061 | 0.091 | 7.6 | 1.5 |
| Compound 9 | 0.024 | 0.21 | 3.3 | 8.7 |
| Compound 10 | 0.017 | 0.24 | 2.9 | 14 |
| Compound 11 | 0.13 | 0.016 | 43 | 0.13 |
| Compound 15 | 0.079 | 0.041 | 17 | 0.52 |
| Compound 45 | 0.11 | 0.024 | 28 | 0.21 |
| Compound 47 | 0.052 | 0.010 | 68 | 0.20 |
| Compound 67 | 0.044 | 0.074 | 9.4 | 1.7 |
| Compound 68 | 0.079 | 0.065 | 11 | 0.82 |
| Compound 69 | 0.13 | 0.070 | 9.9 | 0.53 |
| Compound 70 | 0.0090 | 0.13 | 5.4 | 15 |

a) on-rate from binding kinetics assay. b) off-rate from binding kinetics assay. c) dissociation half-life ln(2)/k_{off}. d) K₁ determined from binding kinetic assay k_{off}/kₒₙ

TABLE 10
Summary of cellular data

| | HCC15 | JIMT-1 | NIH:OVCAR3 |
|---|---|---|---|
| Compound | IC₅₀ (μM) | IC₅₀ (μM) | IC₅₀ (μM) |
| Compound 2 | 0.13 | 0.48 | |
| Compound 9 | 0.11 | 0.10 | 0.20 |
| Compound 10 | 0.12 | 0.13 | 0.21 |
| Compound 11 | 0.013 | 0.0091 | 0.023 |
| Compound 13 | 0.0030 | 0.0047 | 0.0045 |
| Compound 15 | 0.012 | 0.012 | 0.018 |
| Compound 29 | 0.025 | 0.023 | 0.021 |
| Compound 45 | 0.0039 | 0.0069 | 0.0055 |
| Compound 47 | 0.0092 | 0.016 | 0.013 |
| Compound 61 | 0.015 | 0.015 | 0.016 |
| Compound 62 | 0.013 | 0.0095 | 0.013 |
| Compound 64 | 0.12 | 0.12 | 0.12 |
| Compound 67 | 0.051 | 0.066 | 0.047 |
| Compound 68 | 0.028 | 0.030 | 0.021 |
| Compound 69 | 0.029 | 0.038 | 0.032 |
| Compound 71 | 0.012 | 0.020 | 0.013 |
| Compound 72 | 0.016 | 0.024 | 0.025 |
| Compound 73 | 0.010 | 0.042 | 0.014 |
| Compound 79 | 0.0054 | 0.0078 | 0.0083 |
| Compound 87 | 0.043 | 0.090 | 0.049 |
| Compound 89 | 0.034 | 0.048 | 0.051 |
| Compound 90a | 0.0074 | 0.011 | 0.014 |
| Compound 90b | 0.013 | 0.020 | 0.021 |
| Compound 91 | 0.029 | 0.035 | 0.040 |
| Compound 95 | 0.037 | 0.068 | 0.051 |
| Compound 96 | 0.017 | 0.044 | 0.028 |
| Compound 97 | 0.037 | 0.11 | 0.072 |
| Compound 100 | 0.045 | 0.060 | 0.080 |
| Compound 101 | 0.016 | 0.027 | 0.027 |
| Compound 106 | 0.018 | 0.024 | 0.025 |
| Compound 110 | 0.0033 | 0.0068 | 0.0044 |
| Compound 116 | 0.0036 | 0.0063 | 0.0039 |
| Compound 117 | 0.010 | 0.014 | 0.013 |
| Compound 130 | 0.012 | 0.029 | 0.019 |

### Assessment of In Vivo Activity

OVCAR-3 (ATCC) tumor cells were maintained in vitro in RPMI-1640 medium supplemented with 20% fetal bovine serum, 0.01 mg/mL bovine insulin and 1% Anti-Anti at 37° C. in an atmosphere of 5%/CO₂ in air. HCC15 (DSMZ) tumor cells were maintained in vitro in RPMI 1640 medium supplemented with 10% fetal bovine serum and 1% Anti-Anti at 37° C. in an atmosphere of 5% CO₂ in air.

The tumor cells were sub-cultured twice weekly. The cells growing in an exponential growth phase were harvested and counted for tumor inoculation.

Tumor cells (10×106) in 0.2 mL of PBS mixed with Matrigel (50:50) were inoculated subcutaneously on the right flank of each mouse. When the average tumor volume reached 110-175 mm³, animals were randomized into groups of 10 and treatment started. OVCAR-3 cells were implanted in Balb/C nude mice, and HCC15 cell were implanted in SCID Beige mice.

Compounds were dosed once or twice a day (12 h) orally. Tumor Growth Inhibition (TGI) was calculated using the formula: TGI (%)=[1−(TN−T0)/(VN−V0)]×100; TN is the average tumor volume of a treatment group at the indicated timepoint, T0 is the average tumor volume of the treatment group on treatment day 0, VN is the average tumor volume of the vehicle control group at the indicated timepoint, and V0 is the average tumor volume of the vehicle group on treatment day 0. P value was calculated based on tumor size by One-Way ANOVA with GraphPad Prism 9.4.0 compared with the vehicle group, respectively. **** indicates p<0.0001.

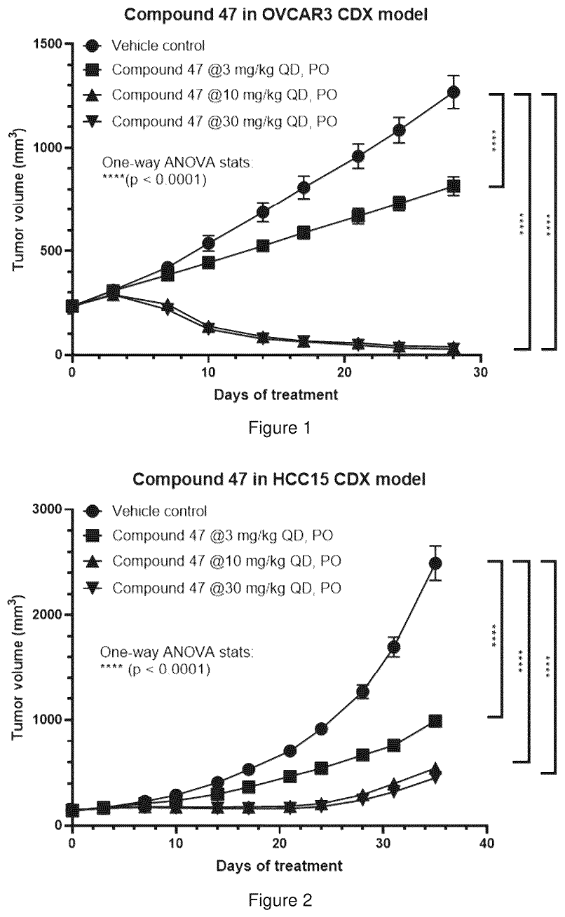

The tumor volume of vehicle- and compound-treated mice as a function of time after start of treatment and the results of treatments with selected compounds on SCID Beige mice or nude mice implanted with HCC15 or OVCAR-3 are shown in FIGS. 1 and 2. The TGI calculated for treatments with selected compounds are shown in in Table 11.

TABLE 11

| Compound | Model | FIG. | Dose | TGI |
|---|---|---|---|---|
| Compound 47 | OVCAR3 | 1 | 3 mg/kg QD PO | 44 ± 11% |
| Compound 47 | OVCAR3 | 1 | 10 mg/kg QD PO | 119 ± 1% |
| Compound 47 | OVCAR3 | 1 | 30 mg/kg QD PO | 120 ± 1% |
| Compound 47 | HCC15 | 2 | 3 mg/kg QD PO | 64 ± 7% |
| Compound 47 | HCC15 | 2 | 10 mg/kg QD PO | 83 ± 4% |
| Compound 47 | HCC15 | 2 | 30 mg/kg QD PO | 87 ± 4% |

1. A compound of Formula (I): 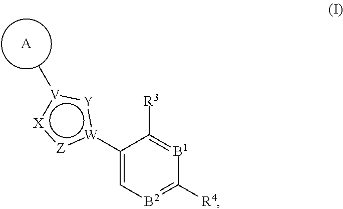
or a pharmaceutically acceptable salt thereof, wherein:
X and Z are independently O, N, or CH;
Y is NH, N, or CH;
V and W are independently N or C;
wherein at least one of X and Z is N or Y is NH;
Ring A is 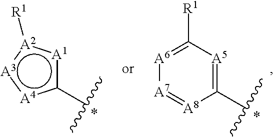
wherein
one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O;
wherein “*” indicates the point of attachment to V;
B¹ and B² are each independently N, CH or CR^{B}, wherein R^{B} is halogen;
R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 10-membered heterocycloalkyl;
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or azepanyl, or
wherein the piperidinyl, pyrrolidinyl, or azepanyl are optionally substituted with a C₁₋₂ alkylene to form a bridged piperidinyl, pyrrolidinyl, or azepanyl ring system,
wherein the piperidinyl, the pyrrolidinyl, the azepanyl, or the spirocyclic, fused, or bridged bicyclic ring system formed by the C₃₋₁₀ cycloalkyl, 3- to 10-membered heterocycloalkyl, or C₁₋₂ alkylene with piperidinyl, pyrrolidinyl, or azepanyl is optionally substituted with one or more substituents independently selected from the group consisting of C₁-C₃ alkyl, C₁-C₃ haloalkyl, and halo;
R⁴ is H, halo, cyano, —OH, —NO₂, —C(O)NR^{c1}R^{c2}, —N^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH,
wherein R⁴ is not H when X is N, Y is N, and Z is O; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl, and wherein each C₃₋₁₀ cycloalkyl is optionally substituted with one or more substituents independently selected from the group consisting of C₁-C₆ alkylene-OH.

2. The compound of claim 1, or a pharmaceutically acceptable salt thereof, wherein X is N.

3. The compound of claim 1, or a pharmaceutically acceptable salt thereof, wherein Z is N.

4. The compound of claim 1, or a pharmaceutically acceptable salt thereof, wherein Y is NH.

5. The compound of claim 1, or a pharmaceutically acceptable salt thereof, wherein the ring

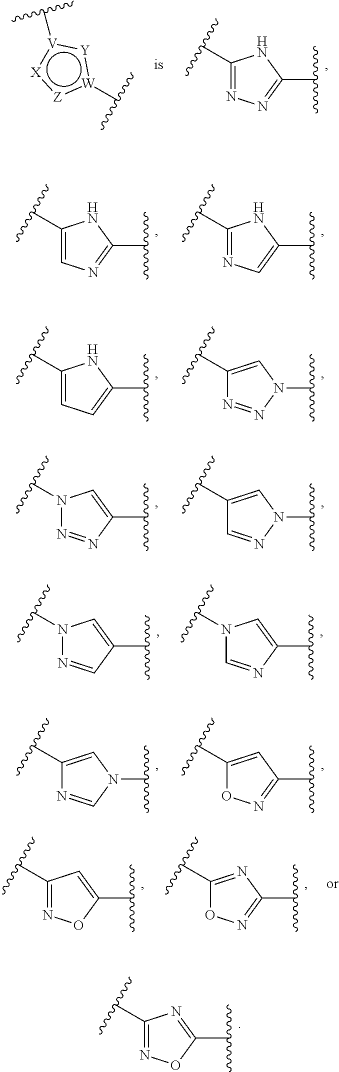

6. The compound of claim 1, or a pharmaceutically acceptable salt thereof, wherein the ring

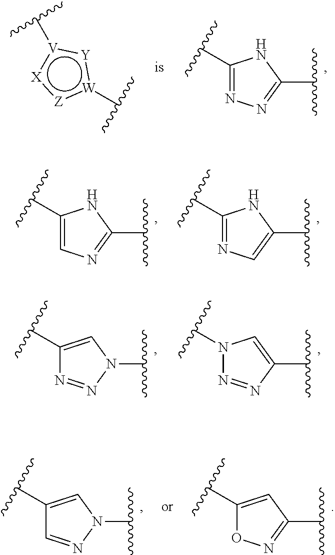

7. The compound of claim 1, or a pharmaceutically acceptable salt thereof, wherein the compound of Formula (I) is a compound of Formula (I-a):

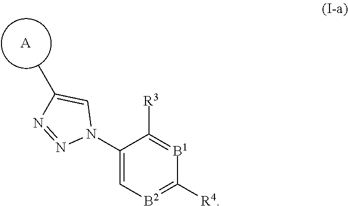

8. The compound of claim 1, or a pharmaceutically acceptable salt thereof, wherein the compound of Formula (I) is a compound of Formula (I-b):

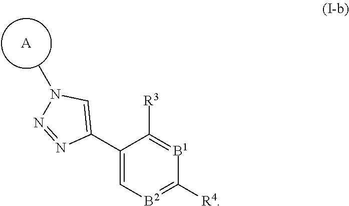

9. The compound of claim 1, or a pharmaceutically acceptable salt thereof, wherein the compound of Formula (I) is a compound of Formula (I-c):

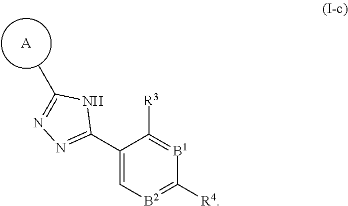

10. The compound of claim 1, or a pharmaceutically acceptable salt thereof, wherein Ring A is

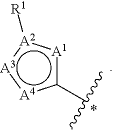

11. The compound of claim 1, or a pharmaceutically acceptable salt thereof, wherein Ring A is

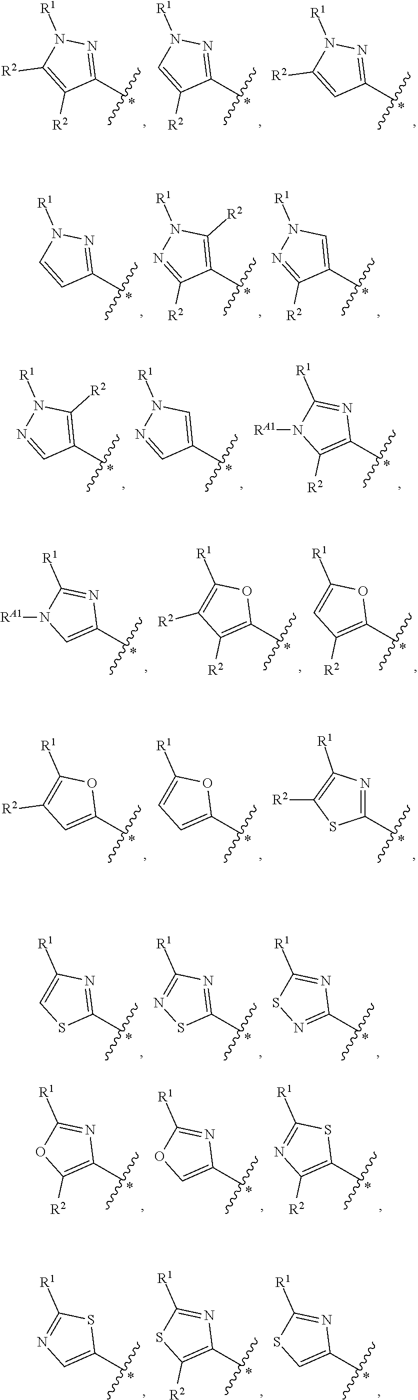

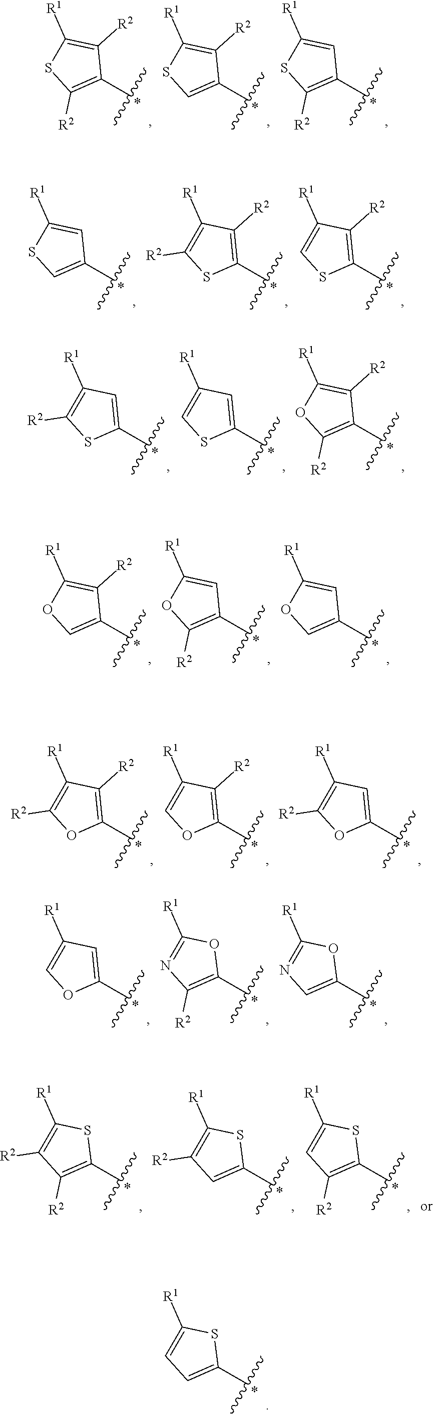

12. The compound of claim 1, or a pharmaceutically acceptable salt thereof, wherein Ring A is

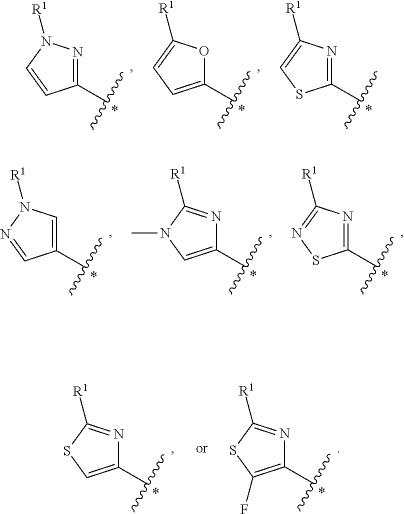

13. The compound of claim 1, or a pharmaceutically acceptable salt thereof, wherein Ring A is

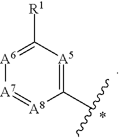

14. The compound of claim 1, or a pharmaceutically acceptable salt thereof, wherein Ring A is

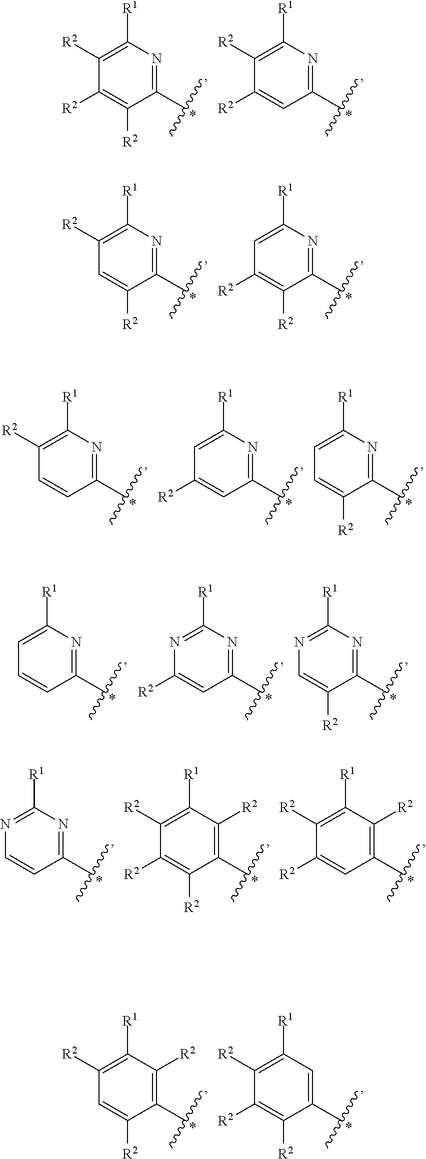

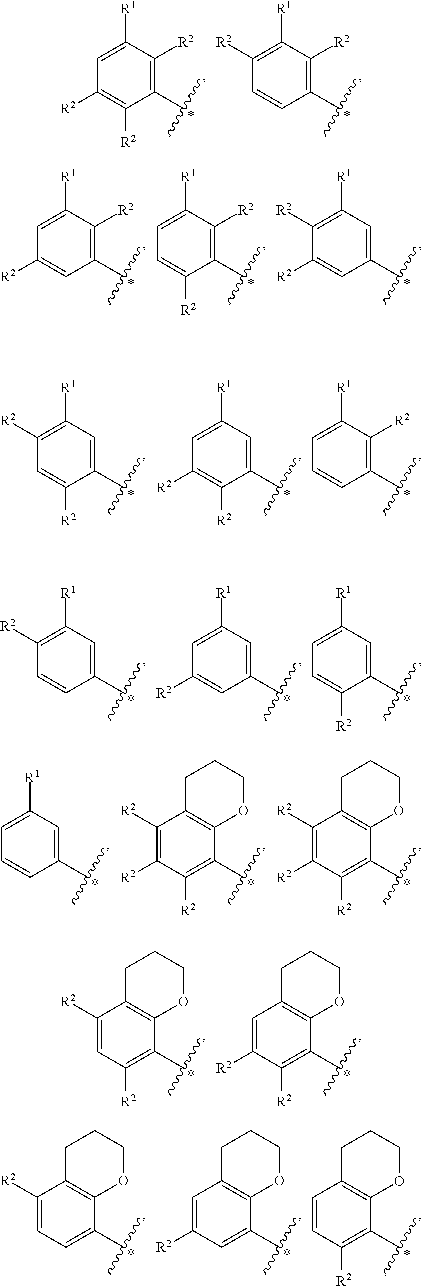

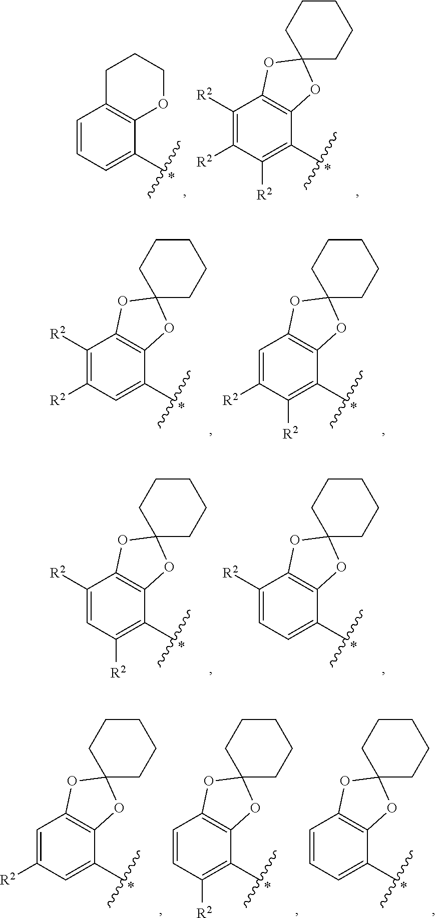

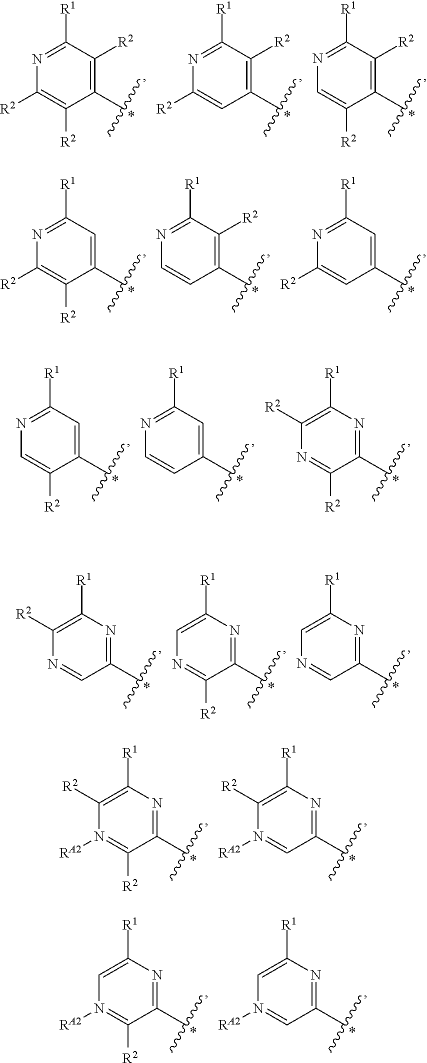

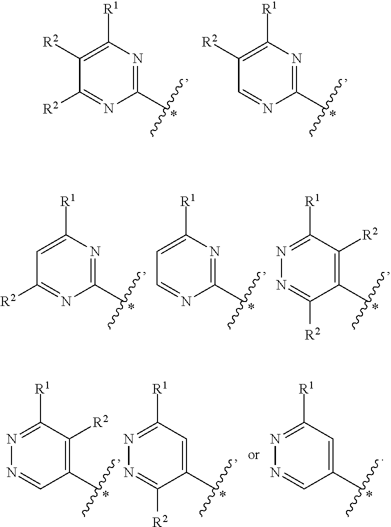

15. The compound of claim 1, or a pharmaceutically acceptable salt thereof, wherein Ring A is

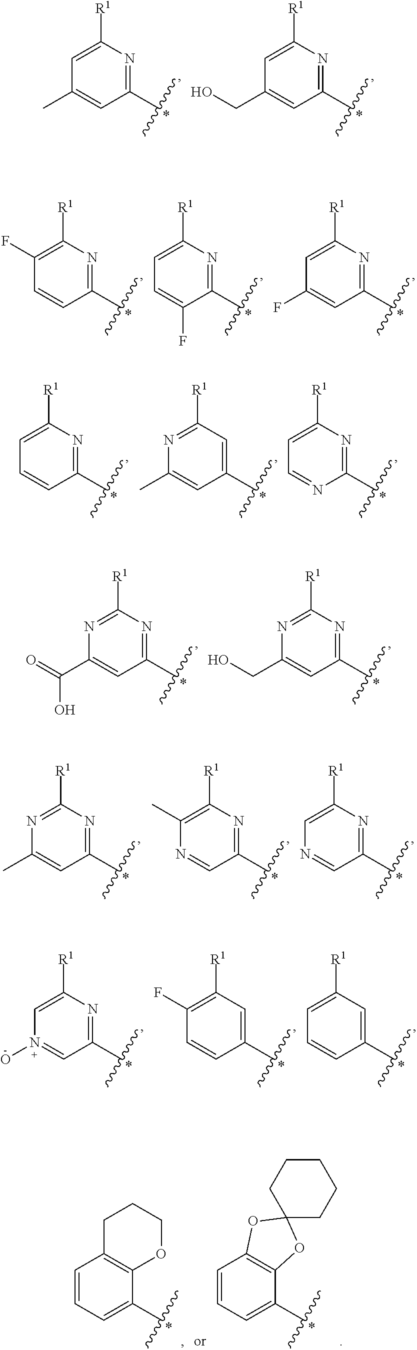

16. The compound of claim 1, or a pharmaceutically acceptable salt thereof, wherein R¹ is C₁₋₆ alkyl, C₃₋₆ cycloalkyl, C₃₋₁₀ cycloalkenyl, 3- to 10-membered heterocycloalkyl, —NR^{a6}R^{a7}, —OR^{a10}, —S(O)₂NR^{a14}R^{a15}, or —S(O)₂R^{a16},
wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl.

17. The compound of claim 1, or a pharmaceutically acceptable salt thereof, wherein R¹ is

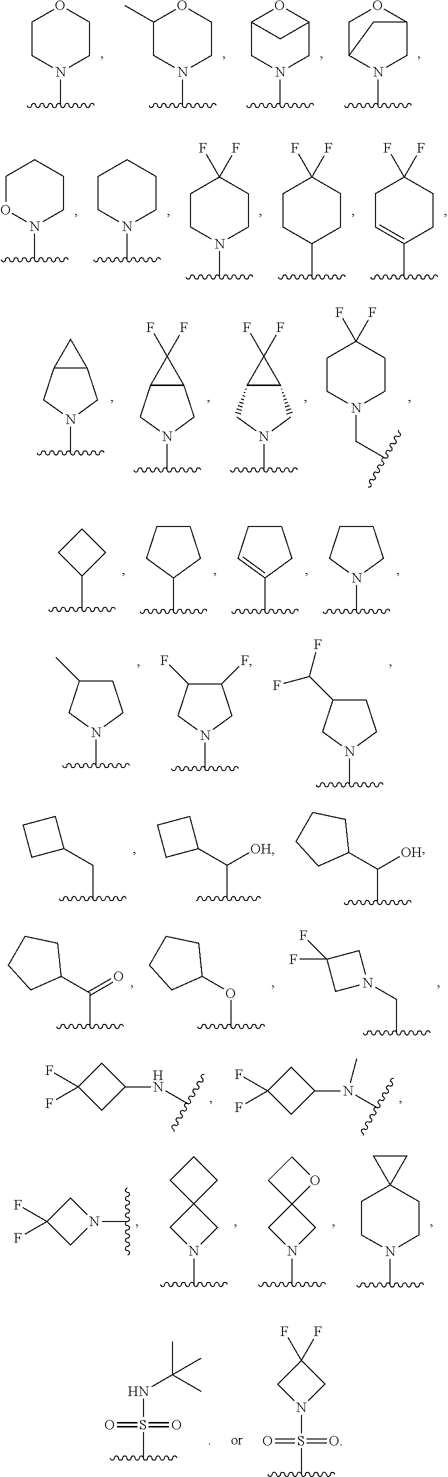

18. The compound of claim 1, or a pharmaceutically acceptable salt thereof, wherein the ring

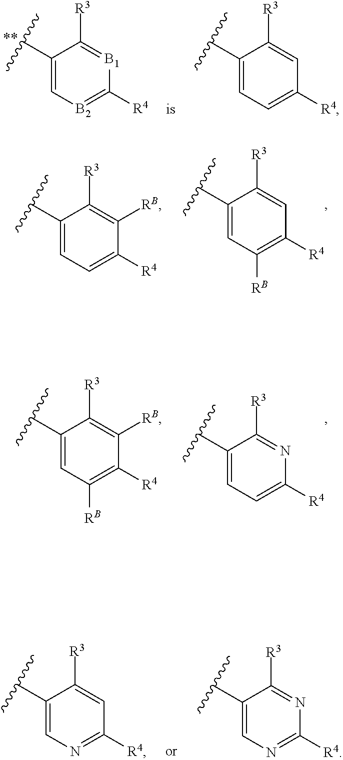

19. The compound of claim 1, or a pharmaceutically acceptable salt thereof, wherein the ring

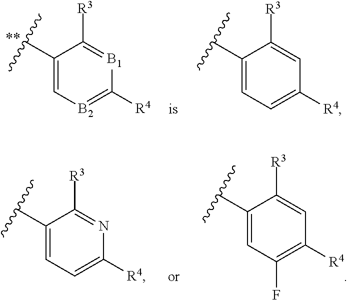

20. The compound of claim 1, or a pharmaceutically acceptable salt thereof, wherein R³ is piperidinyl, wherein the piperidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, or wherein the piperidinyl is optionally substituted with a C₁₋₂ alkylene to form a bridged piperidinyl ring system, wherein the piperidinyl, or the spirocyclic, fused, or bridged bicyclic ring system formed by the C₃₋₁₀ cycloalkyl, 3- to 10-membered heterocycloalkyl, or C₁₋₂ alkylene with piperidinyl is optionally substituted with one or more substituents independently selected from the group consisting of C₁-C₃ alkyl, C₁-C₃ haloalkyl, and halo.

21. The compound of claim 1, or a pharmaceutically acceptable salt thereof, wherein R³ is

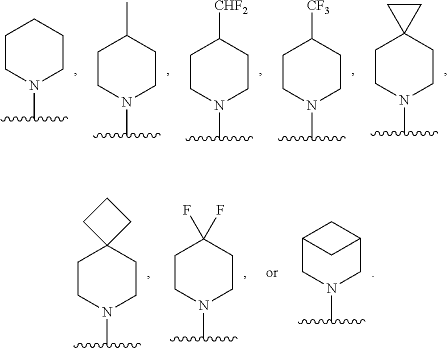

22. The compound of claim 1, or a pharmaceutically acceptable salt thereof, wherein R³ is pyrrolidinyl, wherein the pyrrolidinyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the pyrrolidinyl, or wherein the pyrrolidinyl is optionally substituted with a C₁₋₂ alkylene to form a bridged pyrrolidinyl ring system, wherein the pyrrolidinyl, or the spirocyclic, fused, or bridged bicyclic ring system formed by the C₃₋₁₀ cycloalkyl, 3- to 10-membered heterocycloalkyl, or C₁₋₂ alkylene with the pyrrolidinyl is optionally substituted with one or more substituents independently selected from the group consisting of C₁-C₃ alkyl, C₁-C₃ haloalkyl, and halo.

23. The compound of claim 1, or a pharmaceutically acceptable salt thereof, wherein R³ is

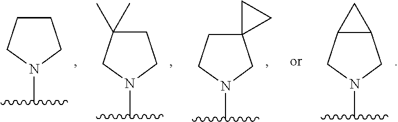

24. The compound of claim 1, or a pharmaceutically acceptable salt thereof, wherein R³ is azepanyl, the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the azepanyl or wherein the azepanyl is optionally substituted with a C₁₋₂ alkylene to form a bridged azepanyl ring system, wherein the azepanyl or the spirocyclic, fused, or bridged bicyclic ring system formed by the C₃₋₁₀ cycloalkyl, 3- to 10-membered heterocycloalkyl, or C₁₋₂ alkylene with the azepanyl is optionally substituted with one or more substituents independently selected from the group consisting of C₁-C₃ alkyl, C₁-C₃ haloalkyl, and halo.

25. The compound of claim 1, or a pharmaceutically acceptable salt thereof, wherein R³ is

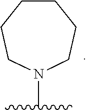 [SMILES: *N1CCCCCC1]

26. The compound of claim 1, or a pharmaceutically acceptable salt thereof, wherein R⁴ is hydrogen, halo, or —NR^{c5}S(O)₂R^{c6}.

27. The compound of claim 1, or a pharmaceutically acceptable salt thereof, wherein R⁴ is H, Br,

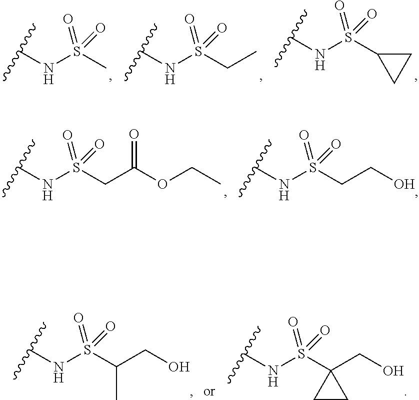

28. The compound of claim 1, or a pharmaceutically acceptable salt thereof, wherein R⁴ is

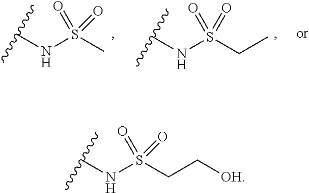

29. A compound of Formula (III) 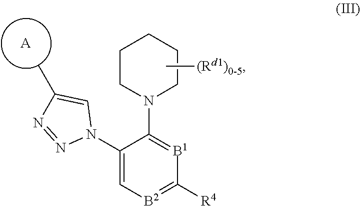
or a pharmaceutically acceptable salt thereof, wherein:
Ring A is 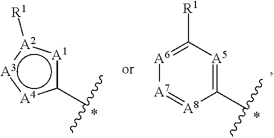
wherein
one, two, or three of A¹, A³, and A⁴ are independently N, NR^{A1}, O, or S, and the remaining one or two of A¹, A³, and A⁴, if present, are independently CH or CR², wherein R^{A1} is H or C₁₋₃ alkyl;
A² is N or C;
A⁵-A⁸ are independently CH, CR², N, or NR^{A2}, wherein at least two of A⁵, A⁶, A⁷, and A⁸ are CH or CR², and the remaining one or two of A⁵, A⁶, A⁷, and A⁸, if present, are N or NR^{A2}, wherein R^{A2} is ═O;
wherein “*” indicates the point of attachment to V;
B¹ and B² are each independently N, CH or CR^{B}, wherein R^{B} is halogen;
R¹ is C₁₋₆ alkyl, C₃₋₆ cycloalkyl, C₃₋₁₀ cycloalkenyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
wherein the C₁-C₆ alkyl of R¹ is optionally substituted with one or more substituents independently selected from the group consisting of halogen, —OH, oxo, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; wherein the C₃₋₁₀ cycloalkenyl of R¹ is optionally substituted with one or more substituents independently selected from the group consisting of halogen; and wherein the 3- to 10-membered heterocycloalkyl of R¹ is optionally substituted with one or more substituents independently selected from the group consisting of halogen, C₁₋₆ alkyl, and C₁₋₆ haloalkyl;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), ═CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl; or
R^{a14} and R^{a15} are taken together with the nitrogen to which they are attached to form a 3- to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein the C₁₋₃ alkyl of R² is optionally substituted by one or more substituents selected from the group consisting of —OH and oxo, and wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 10-membered heterocycloalkyl;
wherein each R^{d1} is independently selected from the group consisting of C₁-C₃ alkyl, C₁-C₃ haloalkyl, and halo; or wherein two R^{d1} are taken together to form a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl; or wherein two R¹ are taken together to form a C₁₋₂ alkylene, wherein the C₁₋₂ alkylene forms a bridged piperidinyl ring system,
wherein the spirocyclic, fused, or bridged bicyclic ring system formed by the C₃₋₁₀ cycloalkyl, 3- to 10-membered heterocycloalkyl, or C₁₋₂ alkylene with the piperidinyl is optionally substituted with one or more substituents independently selected from the group consisting of C₁-C₃ alkyl, C₁-C₃ haloalkyl, and halo;
R⁴ is H, halo, cyano, —OH, —NO₂, —C(O)NR^{c1}R^{c2}, —N^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, an d-C(O)—O—C₁-C₃ alkyl, and wherein each C₃₋₁₀ cycloalkyl is optionally substituted with one or more substituents independently selected from the group consisting of C₁-C₆ alkylene-OH.

30. A compound of Formula (II): 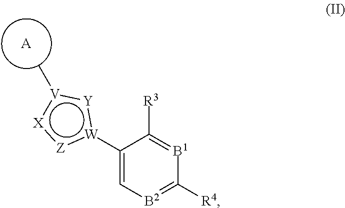
or a pharmaceutically acceptable salt thereof, wherein:
X and Z are independently O, N, or CH;
Y is NH or CH;
V and W are independently N or C;
wherein at least one of X and Z is N or Y is NH;
Ring A is 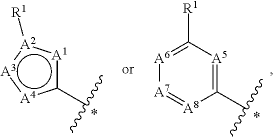
wherein
one or two of A¹, A³, and A⁴ are independently N, O, or S, and the remaining one or two of A¹, A³, and A⁴ are independently CH or CR²;
A² is N or C;
A⁵-A⁸ are independently CH, CR² or N, wherein at least two of A⁵, A⁶, A⁷, and A⁸ are CH or CR², and the remaining one or two of A⁵, A⁶, A⁷, and A⁸, if present, are N;
wherein “*” indicates the point of attachment to V;
B¹ and B² are each independently N or CH;
R¹ is C₁₋₆ alkyl, C₃₋₆ cycloalkyl, 3- to 10-membered heterocycloalkyl, —NR^{a1}C(O)NR^{a2}R^{a3}, —NR^{a4}C(O)OR^{a5}, —NR^{a6}R^{a7}, —N═S(O)R^{a8}R^{a9}, —OR^{a10}, —S(O)R^{a11}, —S(O)(NR^{a12})R^{a13}, —S(O)₂NR^{a14}R^{a15}, —S(O)₂R^{a16}, or —(CR^{a17}R^{a18})₀₋₁C(O)NR^{a19}R^{a20},
wherein the C₁-C₆ alkyl of R¹ is optionally substituted with one or more substituents independently selected from the group consisting of halogen, —OH, cyano, C₃₋₁₀ cycloalkyl, and 3- to 10-membered heterocycloalkyl optionally substituted with one or more halo; wherein the C₃₋₆ cycloalkyl of R¹ is optionally substituted with one or more substituents independently selected from the group consisting of halogen; and wherein the 3- to 10-membered heterocycloalkyl of R¹ is optionally substituted with one or more halogens;
R^{a1}-R^{a20} are each independently hydrogen, C₁₋₆ alkyl, C₂₋₆ alkenyl, C₃₋₁₀ cycloalkyl, C₃₋₁₀ cycloalkenyl, 3- to 10-membered heterocycloalkyl, 3- to 10-membered heterocycloalkenyl, C₆₋₁₄ aryl, or 5- to 12-membered heteroaryl, each optionally substituted with one or more substituents independently selected from the group consisting of halo, cyano, —OH, —O(C₁₋₆ alkyl), C₂₋₆ alkenyl, C₃₋₁₀ cycloalkyl, —S(C₁₋₆ alkyl), =CR^{1a1}R^{1a2}, and C₁₋₆ alkyl optionally substituted with one or more substituents independently selected from the group consisting of halo, —OH, and —O(C₁₋₆ alkyl), wherein R^{1a1} and R^{1a2} are each independently hydrogen or C₁₋₆ alkyl;
or R^{a14} and R^{a15} are taken together with the nitrogen to which they are attached to form a 3-to 10-membered heterocycloalkyl optionally substituted with one or more halo;
each R² is independently halogen, C₁₋₃ alkyl, C₃₋₅ cycloalkyl, cyano, C₁₋₃ alkyloxy, C₃₋₅ cycloalkyloxy, hydroxy, or NR^{b1}R^{b2}, wherein R^{b1} and R^{b2} are independently optionally substituted with C₁-C₃ alkyl or R^{b1} and R^{b2} are taken together with the nitrogen to which they are attached to form a 3- to 6-membered ring; or
R¹ and the R² of A⁵ are taken together with the carbon atoms to which they are attached to form a C₃-C₆ cycloalkyl or a 3- to 6-membered heterocycloalkyl;
R³ is piperidinyl, pyrrolidinyl, or azepanyl, wherein the piperidinyl, the pyrrolidinyl, or the azepanyl is optionally substituted with a C₃₋₁₀ cycloalkyl or 3- to 10-membered heterocycloalkyl, wherein the C₃₋₁₀ cycloalkyl or 3- to 10-membered heterocycloalkyl forms a spirocyclic or fused bicyclic ring system with the piperidinyl, pyrrolidinyl, or the azepanyl, and
wherein the piperidinyl, the pyrrolidinyl, the azepanyl or the spirocyclic or fused bicyclic ring system formed by the C₃₋₁₀ cycloalkyl or 3- to 10-membered heterocycloalkyl with piperidinyl, pyrrolidinyl, or azepanyl is optionally substituted with one or more substituents independently selected from the group consisting of C₁-C₃ alkyl and C₁-C₃ haloalkyl;
R⁴ is hydrogen, halo, cyano, —OH, —NO₂, —C(O)NR^{c1}R^{c2}, —NR^{c3}R^{c4}, —NR^{c5}S(O)₂R^{c6}, —P(O)R^{c7}R^{c8}, —N═S(O)R^{c9}R^{c10}, —S(O)(NR^{c11})R^{c12}, —S(O)₂R^{c13}, or C₁₋₆ alkyl optionally substituted with one or more substituents independently selected from the group consisting of halo and —OH; and
R^{c1}-R^{c13} are each independently hydrogen, C₃₋₁₀ cycloalkyl, or C₁₋₆ alkyl, wherein each C₁-C₆ alkyl of R^{c1}-R^{c13} is optionally substituted with one or more substituents independently selected from the group consisting of halo, —OH, and —C(O)—O—C₁-C₃ alkyl.

31-57. (canceled)

58. The compound of claim 1, or a pharmaceutically acceptable salt thereof, wherein the compound is selected from the group consisting of compounds of Table 1.

59. A pharmaceutical composition comprising a compound of claim 1, or a pharmaceutically acceptable salt thereof, and a pharmaceutically acceptable carrier or excipient.

60. A method of inhibiting KIF18A comprising contacting a cell with an effective amount of a compound of claim 1, or a pharmaceutically acceptable salt thereof.

61. A method of treating a disease or condition mediated by KIF18A in a subject in need thereof, comprising administering to the subject a therapeutically effective amount of a compound of claim 1, or a pharmaceutically acceptable salt thereof.

62. A method of treating cancer in a subject in need thereof, comprising administering to the subject a therapeutically effective amount of a compound of claim 1, or a pharmaceutically acceptable salt thereof.

63. The method of claim 62, wherein the cancer is selected from the group consisting of carcinomas, cancer of the anus, bladder, breast, colon, small intestine, appendix, kidney, renal pelvis, ureter, urothelium, liver, lung, pleura, esophagus, head and neck, nasopharynx, oropharynx, hypopharynx, oral cavity, larynx, biliary tract, gall-bladder, ovary, testicle, germ cell, uterus, pancreas, stomach, cervix, thyroid, prostate, salivary gland, or skin, hematopoietic tumors of lymphoid lineage, hematopoietic tumors of myeloid lineage, hematopoietic tumors of any lineage, myeloma, tumors of mesenchymal origin including sarcomas, tumors of the central and peripheral nervous system, tumor of neuroendocrine origin, tumor of endocrine origin, small cell tumors, tumors of unknown primary, other tumors comprising retinoblastoma, melanoma, seminoma, teratocarcinoma, osteosarcoma, and other cancer-related disorders that are a consequence of cancer presence or progression.
